SEQUENCE LISTING (3 sequences)

SEQ ID NO: 1 - protein, 189 aa, unidentified [sequence omitted]

SEQ ID NO: 2 - protein, 188 aa, unidentified [sequence omitted]

SEQ ID NO: 3 - protein, 5 aa, synthetic construct
EAAAK

(19) United States
(12) Patent Application Publication
(10) Pub. No.: US 2024/0252694 A1
(43) Pub. Date: Aug. 1, 2024

(54) CONJUGATES COMPRISING COVALENT BINDERS FOR TARGETING INTRACELLULAR KRAS G12C PROTEINS

(71) Applicant: RayzeBio, Inc., San Diego, CA (US)

(72) Inventors: Nicholas D. SMITH, San Diego, CA (US); Andrew HUDSON, San Diego, CA (US)

(21) Appl. No.: 18/431,646

(22) Filed: Feb. 2, 2024

Related U.S. Application Data
(63) Continuation of application No. PCT/US22/39589, filed on Aug. 5, 2022.
(60) Provisional application No. 63/230,432, filed on Aug. 6, 2021.
(60) Provisional application No. 63/299,698, filed on Jan. 14, 2022.

(51) Int. Cl. A61K 51/04; A61P 35/00

(52) U.S. Cl. CPC A61K 51/0482; A61K 51/0478; A61K 51/048; A61P 35/00

(57) ABSTRACT

Provided herein are radiopharmaceutical compositions, conjugates and uses thereof. In one aspect, provided herein are covalently modified KRAS proteins comprising a radiolabeled compound or conjugate. In some embodiments, the radiolabeled compound described herein comprises a covalently bound radioisotope. In some embodiments, the radiolabeled conjugate comprises a targeting ligand and a metal chelator configured to bind a radionuclide. The radiolabeled compound or conjugate described herein can form a covalent bond with a cysteine residue (such as G12C) in the mutated KRAS protein. Further provided herein are methods of treating and diagnosing cancer by administering the described radiolabeled compounds, conjugates, and compositions to a subject, thereby forming the covalently modified KRAS G12C protein.

### CROSS-REFERENCE

This application is the U.S. By-pass Continuation of International Application No. PCT PCT/US2022/039589, filed Aug. 5, 2022, which claims the benefit of U.S. Provisional Application No. 63/230,432, filed on Aug. 6, 2021, and U.S. Provisional Application No. 63/299,698, filed on Jan. 14, 2022; each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 1, 2024, is named 59541-719-301-SL.xml and is 3,811 bytes in size.

### BACKGROUND

In the United States, cancer is the leading cause of death for those under 65 years of age, and it accounted for about 21% of all death in 2018. Traditional radiotherapies such as external beam radiation therapy have been used for decades as a standard-of-care treatment for diagnosed cancer patients. While some patients respond to external beam radiation therapy, many others do not. Further, metastasis and circulating tumor cells can spread and remain in the bloodstream or bodily fluids after standard-of-care treatment and lead to resistance to therapy. The presence of cancer cells in various parts of the body reduces the therapeutic efficacy of traditional radiotherapies. Accordingly, strategies for targeted radiotherapies are being developed, and there remains a need for targeted radiotherapies that have the desired affinity, stability, and exertion profile.

### SUMMARY

Kirsten rat sarcoma vial oncogene (KRAS), member of the RAS superfamily, is one of the most prevalent oncogenes in cancer. As a GTP-binding protein that links receptor tyrosine kinase activation to intracellular signaling. KRAS mutations favor the GTP-bound active state and constitutive activation of downstream effects including differentiation, proliferation and survival. Presence of KRAS mutations have been shown to be a negative prognostic factor in multiple cancer types including, for example, lung and colorectal cancers.

In one aspect, described herein are targeted radiotherapy (TRT) conjugates that engage and bind to intracellular oncology target KRAS protein. In some embodiments, the conjugates described herein form a covalent bond with mutated KRAS protein, for example, at G12C residue. In some embodiments, the conjugates described herein are useful as therapeutic agents (such as therapeutics for treating cancer). In some embodiments, the conjugates described herein are useful as theranostic agents. In some embodiments, the conjugates described herein are used to confirm the expression of an intracellular oncology target protein in a subject. In some embodiments, the conjugates described herein can have their pharmacokinetic properties monitored to aid in patient care.

In one aspect, provided herein are KRAS proteins that have been covalently modified with a radiolabeled compound comprising a covalently bonded radioisotope. In some embodiments, the KRAS protein is covalently modified in vivo by the radiolabeled compound comprising the covalently bonded radioisotope. Also provided herein are methods of making and using the covalently modified KRAS protein for treatment and diagnosis of cancer and other proliferative diseases. In some embodiments, the radiolabeled compound has an electrophilic functional group, such as the structure of Formula (Ia), (Tb) or (Id). In some embodiments, the electrophilic functional group has a structure of Formula (Ic).

In one aspect, provided herein is a covalently modified KRAS protein comprising a glycine to cysteine amino acid substitution at residue 12 (G12C), and a radiolabeled compound comprising a covalently bonded radioisotope, wherein the radiolabeled compound is bonded to the KRAS protein at the cysteine residue 12 of the KRAS protein through a covalent bond, and wherein residue position numbering of the KRAS protein is based on SEQ ID NO:1 or SEQ ID NO: 2 as a reference sequence.

In one aspect, described herein is a radiopharmaceutical conjugate comprising (a) a targeting ligand that is configured to form a covalent bond with a KRAS protein at the G12C position, wherein residue position numbering of the KRAS protein is based on SEQ ID NO: 1 or SEQ ID NO: 2, and (b) a radionuclide, wherein the radionuclide is iodine-131 or astatine-211.

In one aspect, described herein is a radiopharmaceutical conjugate comprising (a) a targeting ligand that is covalently bound to an intracellular mutated KRAS protein at the G12C position, wherein residue position numbering of the KRAS protein is based on SEQ ID NO: 1 or SEQ ID NO: 2, and (b) a radionuclide. In some embodiments, the radionuclide is selected from astatine-211, astatine-217, actinium-225, americium-243, radium-223, lead-212, lead-203, copper-64, copper-67, copper-60, copper-61, copper-62, bismuth-212, bismuth-213, gallium-68, gallium-67, dysprosium-154, gadolinium-148, gadolinium-153, samarium-146, samarium-147, samarium-153, terbium-149, thorium-227, thorium-229, iron-59, yttrium-86, indium-111, holmium-166, technetium-94, technetium-99^{m}, yttrium-90, lutetium-177, terbium-161, rhenium-186, rhenium-188, cobalt-55, scandium-43, scandium-44, scandium-47, dysprosium-166, fluorine-18, and iodine-131.

In one aspect, described herein is a radiopharmaceutical conjugate comprising: (a) a targeting ligand that covalently binds to an intracellular KRAS protein, wherein the intracellular KRAS protein is mutated, and wherein the targeting ligand comprises a structure of Formula (III), or a salt or solvate thereof,

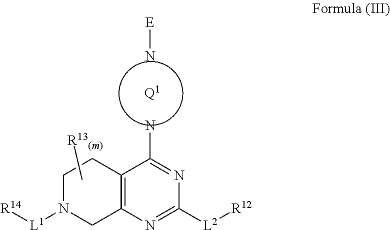

wherein
ring Q¹ is a 4-12 membered saturated or partially saturated monocyclic or bicyclic ring, wherein the monocyclic or bicyclic ring is optionally substituted;
   L¹ is a bond, —C(═O)—, or optionally substituted C₁-C₃ alkylene;
   L² is a bond, —C(═O)—, O, S or NR¹⁵;
E is

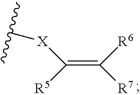

X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₅ heterocycloalkyl, wherein each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₇ heterocycloalkyl, or heteroaryl, wherein each of the alkyl, alkoxyl, heteroalkyl, cycloalkyl or heterocycloalkyl is optionally substituted; or
R⁶ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆, alkoxyl, C₁-C₆ heteroalkyl, C₃-C₆ cycloalkyl, C₂-C₆ heterocycloalkyl, or heteroaryl, wherein each of the alkyl, alkoxyl, heteroalkyl, cycloalkyl, heterocycloalkyl, or heteroaryl is optionally substituted;
R¹² is hydrogen, C₁-C₆ alkyl, C₁-C₆ heteroalkyl, -L³-NR¹⁵R¹⁵, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
L³ is C₁-C₄, alkylene or C₁-C₄ heteroalkylene, each of which is optionally substituted;
each R¹³ is independently OH, halogen, oxo, substituted or unsubstituted C₁-C₆ alkyl, or substituted or unsubstituted C₁-C₆ heteroalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or C₁-C₃ alkyl;
each R^{15′} is independently hydrogen, acyl, C₁-C₃ alkyl, C₁-C₃ heteroalkyl or C₁-C₃ hydroxyalkyl;
m is 0, 1, or 2,
wherein the structure of Formula (III) is attached to the rest of the conjugate at any suitable position; and
(b) a radionuclide.
In some embodiments, the radionuclide is covalently bound to the structure of Formula (III). In some embodiments, at least one of L¹, L², R¹², R¹³ and R¹⁴ comprises a covalently bonded radioisotope R*. In some embodiments. R* is selected from a radioisotope in Table 6C or Table 6D.

In some embodiments, the targeting ligand comprises a structure of Formula (IIIa), or a salt or solvate thereof.

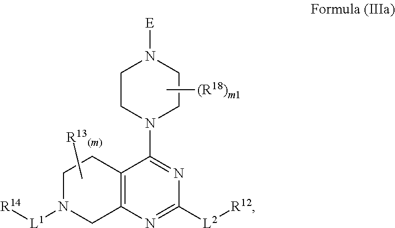

wherein
each R¹⁸ is independently halogen, oxo, C₁-C₆ alkyl, C₁-C₆ aminoalkyl, C₁-C₆ haloalkyl, C₂-C₈ alkenyl, C₂-C₆ alkynyl, C₁-C₆ heteroalkyl, —CN, —C(O)OR¹⁵, —C(O)N(R¹⁵)(R¹⁵), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, and C₂-C₅ heterocycloalkyl; and
m1 is 0, 1, 2, or 3.

In some embodiments, the structure of Formula (III) is attached to the rest of the conjugate through R¹². In some embodiments, R¹⁴ comprises the radionuclide and the radionuclide is a covalently bonded, wherein the radionuclide is selected from fluorine-18 (¹⁸F), iodine-131 (¹³¹), iodine-123 (¹²³I), iodine-124 (¹²⁴I), iodine-125 (¹²⁵I), and astatine-211 (²¹¹At).

In some embodiments the targeting comprises a structure of Formula (IIIa-1) or Formula (IIIa-2), or a salt or solvate thereof,

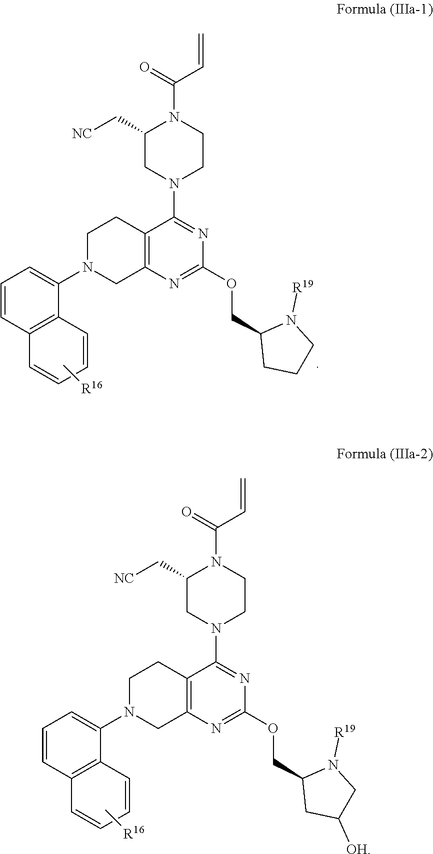

In some embodiments, the structure of Formula (IIIa-1) or Formula (IIIa-2) is attached to the rest of the conjugate through R¹⁹ or R¹⁶. In some embodiments, R¹⁹ comprises the radionuclide and the radionuclide is a covalently bonded, wherein the radionuclide is selected from fluorine-18 (¹⁸F), iodine-131 (¹³¹I), iodine-123 (¹²¹I), iodine-124 (¹²⁴I), iodine-125 (¹²⁵I), and astatine-211 (²¹¹At). In some embodiments, R¹⁶ comprises the radionuclide and the radionuclide is a covalently bonded, wherein the radionuclide is selected from fluorine-18 (¹⁸F), iodine-131 (¹³¹I), iodine-123 (¹²³I), iodine-124 (¹²⁴I), iodine-125 (¹²⁵I), and astatine-211 (²¹¹At).

In some embodiments, the targeting ligand is configured to form a covalent bond with a KRAS protein at the G12C position, wherein residue position numbering of the KRAS protein is based on SEQ ID NO:1 or SEQ ID NO:2.

In one aspect, described herein is a radiopharmaceutical conjugate comprising: (a) a targeting ligand that is configured to form a covalent bond with a KRAS protein at the G12C position, wherein residue position numbering of the KRAS protein is based on SEQ ID NO:1 or SEQ ID NO: 2; comprising a structure of Formula (IV), or a salt or solvate thereof,

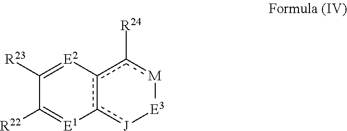

wherein
E¹ and E² are each independently N or CR²¹,
E³ is CR²⁸R²⁹, C═CR²⁸R²⁹, C═O, C═S, or C═NR²⁸;
J is N, NR³⁰, or CR³⁰;
M is N, NR¹⁵, or CR¹⁵;
[symbol] is a single or double bond as necessary to give every atom its normal valence;
R²¹ is independently hydrogen, hydroxyl, cyano, halogen, C₁-C₆ alkyl, C₁-C₆ alkoxyl, or C₁-C₆ heteroalkyl, wherein each of the alkyl, alkoxy, and heteroalkyl are optionally substituted;
R²² is hydrogen, halogen, C₁-C₆ alkyl, C₂-C₆ alkenyl, C₂-C₆ alkynyl, OR²², N(R²²)₂, cycloalkyl, heterocycloalkyl, aryl, or heteroaryl, wherein each of the alkyl, alkylene, alkenyl, alkynyl, cycloalkyl, heterocycloalkyl, aryl, and heteroaryl is optionally substituted, and each R²² is independently hydrogen, C₁-C₆ alkyl, cycloalkyl, heterocycloalkyl, C₁-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted, or two R²² together with the nitrogen atom to which they are attached, form a 3-7-membered ring;
R²³ is hydrogen, halogen, C₁-C₆ alkyl, C₁-C₆ alkoxy, C₂-C₆ alkenyl, C₂-C₆ alkynyl, cycloalkyl, heterocycloalkyl, aryl, heteroaryl, wherein each of the alkyl, alkoxy, cycloalkyl, heterocycloalkyl, alkenyl, alkynyl, aryl, and heteroaryl is optionally substituted;
R²⁴ is

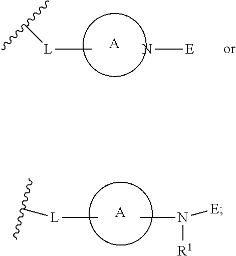

ring A is an optionally substituted 4-7 membered monocyclic ring or optionally substituted 6-11 membered bicyclic, bridged, fused, or spiro ring;
R¹ is hydrogen, optionally substituted C₁-C₆ alkyl, optionally substituted C₁-C₆ heteroalkyl, optionally substituted C₃-C₆ cycloalkyl, or optionally substituted C₂-C₅ heterocycloalkyl;
L¹ is a bond, C₁-C₆ alkylene, C₁-C₆ heteroalkylene, S. O, or NH;
E represents a structure of Formula (Ic).

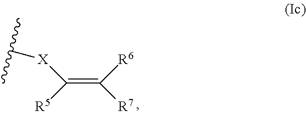

wherein,
X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₇ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently hydrogen, hydroxyl, C₁-C₆ alkyl, C₁-C₆ alkoxy, cyan, nitro, or C₃-C₆ cycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, —C₁-C₆alkylene-cycloalkyl, —C₁-C₆alkylene-heterocycloalkyl, —C₁-C₆alkylene-aryl, —C₁-C₆alkylene-heteroaryl, —C₁-C₆heteroalkylene-cycloalkyl, —C₁-C₆heteroalkylene-heterocycloalkyl, —C₁-C₆heteroalkylene-aryl, —C₁-C₆alkylene-heteroaryl wherein each of the alkyl, alkoxy, heteroalkyl, cycloalkyl, heterocycloalkyl, aryl, and heteroaryl is optionally substituted;
R³³ is hydrogen, C₁-C₆ alkyl, C₁-C₆, heteroalkyl, or C₃-C₆ cycloalkyl, wherein each of the alkyl, heteroalkyl, and cycloalkyl is optionally substituted; and
(b) a radionuclide.

In some embodiments, the radionuclide is covalently bound to the structure of Formula (IV). In some embodiments, at least one of R²¹, R²², R²³, R²⁴, and R³⁰ comprises the radionuclide. In some embodiments, the radionuclide is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, the radioisotope is iodine-131 (¹³¹I).

In some embodiments, the targeting ligand comprises a structure of Formula (IVa), or a salt or solvate thereof,

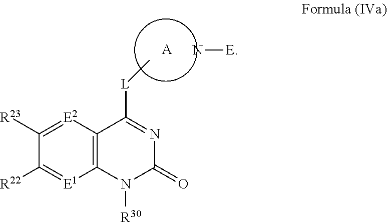

In some embodiments, the targeting ligand comprises a structure of Formula (IVb) or Formula (IVc), or a salt or solvate thereof,

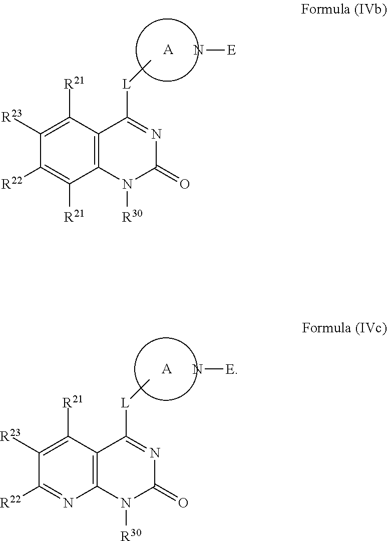

In some embodiments, R²² comprises the radionuclide. In some embodiments, R²² is

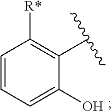

and R* is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, R²³ comprises the radionuclide, and R²³ is halogen and the halogen is the radionuclide selected from fluorine-18 (¹⁸F), iodine-131 (¹³¹I), iodine-123 (¹²³I), iodine-124 (¹²⁴I), iodine-125 (¹²⁵I), and astatine-211 (²¹¹At). In some embodiments, R²³ is ¹³¹I.

In one aspect, described herein is a radiopharmaceutical conjugate comprising: a) a structure of Formula (III), Formula (IIIa), Formula (IIIa-1), Formula (IIIa-2), Formula (IV), Formula (IVa), Formula (IVb), or Formula (IVc), b) a radionuclide R, and c) a linker covalently bonded to the radionuclide R* and the structure of Formula (III), Formula (IIIa), Formula (IIIa-1), Formula (IIIa-2), Formula (IV). Formula (IVa), Formula (IVb), or Formula (IVc), or a salt or solvate thereof. In some embodiments, R* is selected from a radioisotope in Table 6C or Table 6D.

In some embodiments, radiolabeled compound comprising a structure of Formula (IIIb), or a salt or solvate thereof,

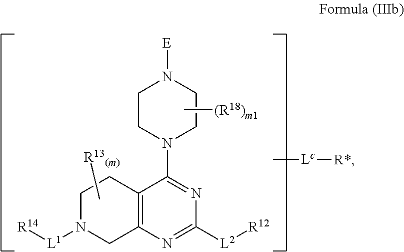

wherein
L^{C} is a linker comprising 1 to 20 groups independently selected from —CR^{b}R^{b}—, —C(═O)—, —S(═O)—, —S(═O)₂—, —NR^{a}—,

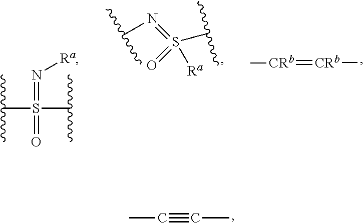

—O—, —S—, —C(═O)O—, —OC(═O)—, —C(═O)NR^{a}—, —NR^{a}C(═O)—, —S(═O)₂NR^{a}—, —NR^{a}S(═O)₂—, —NR^{a}C(═O)NR^{a}—, —NR^{a}C(═O)O—, —OC(═O)NR^{a}—, arylene, heteroarylene;
each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
each R^{b} is independently hydrogen, halogen, —CN, —NO₂. —OR^{a}, —SR^{a}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₉heterocycloalkyl, aryl, or heteroaryl;
R¹² is C₁-C₆ alkyl, C₁-C₆ heteroalkyl, -L³-NR¹⁵—, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At); and
E. L¹, L², L³, R¹³, R¹⁴, R¹⁸, m and m1 have the meaning defined above in Formula (III).

In some embodiments, the radiolabeled compound comprises a structure of Formula (IIIc):

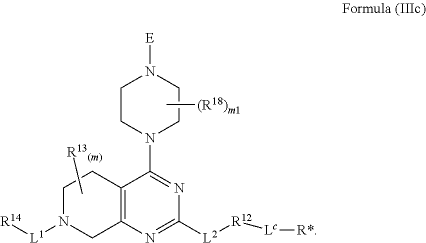

In some embodiments, the radiolabeled compound comprises a structure of formula (IVd), or a salt or solvate thereof,

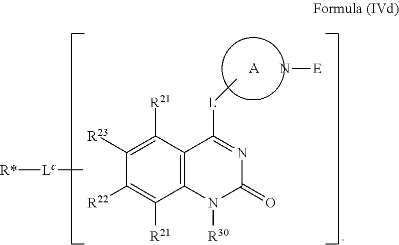

wherein
L^{C} is a linker comprising 1 to 20 groups independently selected from —CR^{b}R^{b}—, —C(═O)—, —S(═O)—, —S(═O)₂—, —NR^{a}—,

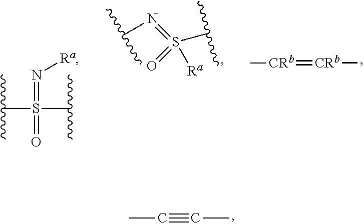

—O—, —S—, —C(═O)O—, —OC(═O)—, —C(═O)NR^{a}—, NR^{a}C(═O), —S(═O)₂NR^{a}—, —NR^{a}S(═O)₂—, —NR^{a}C(═O)NR^{a}—, —NR^{a}C(═O)O—, —OC(═O)NR^{a}—, arylene, heteroarylene;
each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₉heterocycloalkyl, aryl, or heteroaryl;
each R^{b} is independently hydrogen, halogen, —CN, —NO₂, —OR^{a}, —SR^{a}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At);
R²² is C₁-C₆ alkyl, C₂-C₆ alkenyl, C₂-C₆ alkynyl, cycloalkyl, —C₁-C₃alkylene-cycloalkyl, heterocycloalkyl, —C₁-C₃alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted; and
E, ring A, L, R²¹, R²², R²³, and R³⁰ have the meaning defined above in Formula (IV).

In some embodiments, a radiopharmaceutical conjugate comprises a structure of Formula (IVe).

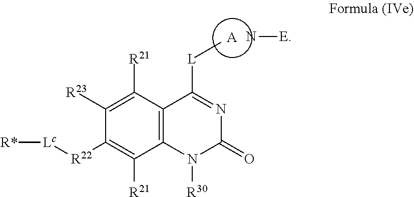

The radiopharmaceutical conjugate comprising a structure of Formula (III), Formula (IIIa). Formula (IIIa-1), Formula (IIIa-2), Formula (IIIb), Formula (IIIc), Formula (IV), Formula (IVa), Formula (IVb), Formula (IVc), Formula (IVd), Formula (IVe) as disclosed herein can covalently bond KRAS G12C and further comprises a covalently bonded radioisotope. In one aspect, provided herein are pharmaceutical compositions comprising a radiopharmaceutical conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), or Formula (X) or a salt or solvate thereof.

In one aspect, described herein is a radiopharmaceutical conjugate comprising: (a) a targeting ligand that covalently binds to an intracellular KRAS protein, wherein the intracellular KRAS protein is mutated, and wherein the targeting ligand comprises a structure of Formula (III) or Formula (IV), further comprising a linker; and a metal chelator.

In some embodiments, the conjugate has a structure of Formula (X):

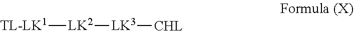

wherein,
TL represents the targeting ligand;
CHL represents the metal chelator, optionally bound to a radionuclide; and
each of LK¹, LK², and LK³ is independently selected from substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₂-C₁₂ alkynylene, substituted or unsubstituted cycloalkylene, substituted or unsubstituted heterocycloalkylene, —(CH₂CH₂O)_{q}, —(OCH₂CH₂)_{q}, —O—, —S—, —S(═O)—, —S(═O)₂—, —S(═O)(═NR^{LK})—, —C(═O)—, —C(═N—OR^{LK})—, —C(═O)O—, —OC(═O)—, —C(═O)C(═O)—, —C(═O)NR^{LK}—, —NR^{LK}C(═O)—, —OC(═O)NR—, —NR^{LK}C(═O)O—, —NR^{LK}C(═O)NR^{LK}—, —C(O)NR^{LK}C(═O)—, —S(O)₂NR—, —NR^{LK}S(═O)₂—, —NR^{LK}—, —N(OR^{LK})—, and a bond;
each R^{LK} is independently hydrogen or substituted or unsubstituted C₁-C₆alkyl; and
q is an integer selected from 1 to 10.

In some embodiments, the conjugate of Formula (X) has the structure of Formula (X-III):

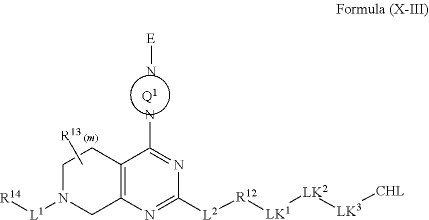

wherein
ring Q¹ is a 4-12 membered saturated or partially saturated monocyclic or bicyclic ring, wherein the monocyclic or bicyclic ring is optionally substituted;
L¹ is a bond, —C(═O)—, or optionally substituted C₁-C₃ alkylene;
L² is a bond, —C(═O)—, O, S or NR¹⁵;
E is

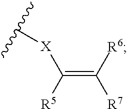

X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆ alkyl, C₁-C₆ alkoxyl, C₁-C₆ heteroalkyl, C₁-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₇ cycloalkyl, C₂-C₇ heterocycloalkyl, C₁-C₉heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₂-C₆ heterocycloalkyl, C₁-C₉heteroaryl, —C₁-C₆alkylene-C₁-C₉ heteroaryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted.
R¹² is C₁-C₆ alkyl, C₁-C₆, heteroalkyl, -L³-NR¹⁵R^{15′}, C₂-C₁₂ heterocycloalkyl, -L³-C₂-C₁₂ heterocycloalkyl, C₃-C₁₀cycloalkyl, -L³-C₃-C₁₀cycloalkyl, aryl, C₁-C₉heteroaryl, -L³-aryl, or -L³-C₁-C₉ heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted;
each R¹³ is independently OH, halogen, oxo, substituted or unsubstituted C₁-C₆ alkyl, or substituted or unsubstituted C₁-C₆ heteroalkyl;
R¹⁴ is hydrogen, C₃-C₁₀cycloalkyl, C₂-C₁₂ heterocycloalkyl, aryl, or C₁-C₉heteroaryl, wherein each of the cycloalkyl, heterocycloalkyl, aryl, and heteroaryl is optionally substituted;
each R¹⁵ is independently hydrogen or C₁-C₃ alkyl;
each R^{15′} is independently hydrogen, acyl, C₁-C₃ alkyl, C₁-C₃ heteroalkyl or C₁-C₃ hydroxyalkyl; and
m is 0, 1, or 2.

In some embodiments, the conjugate of Formula (X) has the structure of Formula (X-V):

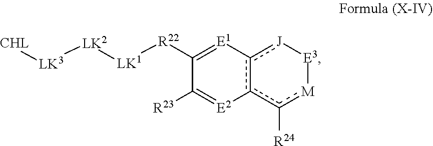

wherein
E¹ and E² are each independently N or CR²¹;
E³ is CR²⁸R²⁹, C═CR²⁸R²⁹, C═O, C═S, or C═NR²⁸;
J is N, NR³⁰, or CR³⁰;
M is N, NR³³, or CR³³;
[symbol] is a single or double bond as necessary to give every atom its normal valence;
R²¹ is independently hydrogen, hydroxyl, cyano, halogen, C₁-C₆ alkyl, C₁-C₃ alkoxyl, or C₁-C₆ heteroalkyl, wherein each of the alkyl, alkoxy, and heteroalkyl are optionally substituted;
R²² is C₁-C₆ alkyl, C₂-C₆ alkenyl, C₂-C₆ alkynyl, OR^{22′}, N(R^{22′})₂, C₃-C₁₀cycloalkyl, C₂-C₁₂ heterocycloalkyl, aryl, or C₁-C₉ heteroaryl, wherein each of the alkyl, alkylene, alkenyl, alkynyl, cycloalkyl, heterocycloalkyl, aryl, and heteroaryl is optionally substituted, and each R is independently hydrogen, C₁-C₆alkyl, C₃-C₁₀cycloalkyl, C₂-C₁₂heterocycloalkyl, C₂-C₆ alkenyl, C₂-C₆ alkynyl, aryl, or C₁-C₉ heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form a 3-7-membered ring;
R²³ is hydrogen, halogen, C₁-C₆ alkyl, C₁-C₆ alkoxy, C₂-C₆ alkenyl, C₂-C₆ alkynyl, C₃-C₁₀cycloalkyl, C₂-C₁₂heterocycloalkyl, aryl, C₁-C₉ heteroaryl, wherein each of the alkyl, alkoxy, cycloalkyl, heterocycloalkyl, alkenyl, alkynyl, aryl, and heteroaryl is optionally substituted;
R²⁴ is

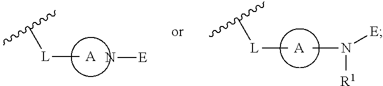

ring A is an optionally substituted 4-7 membered monocyclic ring or optionally substituted 6-11 membered bicyclic, bridged, fused, or spiro ring;
R¹ is hydrogen, optionally substituted C₁-C₆ alkyl, optionally substituted C₁-C₆ heteroalkyl, optionally substituted C₃-C₆ cycloalkyl, or optionally substituted C₂-C₆ heterocycloalkyl;
L is a bond, C₁-C₆ alkylene, C₁-C₆ heteroalkylene, S, O, or NH;
E represents a structure of Formula (Ic),

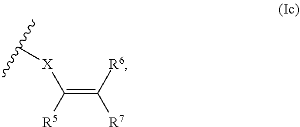

wherein,
X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆alkyl, C₁-C₆alkoxyl, C₁-C₆heteroalkyl, C₃-C₆cycloalkyl, or C₂-C₅heterocycloalkyl, each of the alkyl, alkoxyl, heteroalkyl, cycloalkyl or heterocycloalkyl is optionally substituted, and
R⁷ is hydrogen, cyan, halogen, C₁-C₆alkyl, C₁-C₆ alkoxyl, C₁-C₆ heteroalkyl, C₃-C₇ cycloalkyl, C₂-C₇heterocycloalkyl, C₁-C₉ heteroaryl, —C₁-C₆alkylene-C₁-C₉heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, C₁-C₉heteroaryl, —C₁-C₆alkylene-C₁-C₉heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently hydrogen, hydroxyl, C₁-C₆ alkyl, C₁-C₆ alkoxy, cyano, nitro, or C₃-C₆ cycloalkyl, or R²⁸ and R²⁹ taken together with the carbon atom to which they are attached form a 3-6 membered ring;
R³⁰ is selected from halogen, cyan, C₁-C₆ alkyl, C₁-C₆ alkoxy, C₁-C₆ heteroalkyl, C₁-C₆cycloalkyl, C₂-C₁₂heterocycloalkyl, aryl, C₁-C₉heteroaryl, —C₁-C₆alkylene-C₃-C₆cycloalkyl, —C₁-C₆alkylene-C₂-C₁₂heterocycloalkyl, —C₁-C₆alkylene-aryl, —C₁-C₆alkylene-C₁-C₉heteroaryl, —C₁-C₆heteroalkylene-C₃-C₁₀cycloalkyl, —C₁-C₆heteroalkylene-C₂-C₁₂heterocycloalkyl, —C₁-C₆heteroalkylene-aryl, —C₁-C₆alkylene-C₁-C₉ heteroaryl wherein each of the alkyl, alkoxy, heteroalkyl, cycloalkyl, heterocycloalkyl, aryl, and heteroaryl is optionally substituted; and
R³³ is hydrogen, C₁-C₆ alkyl, C₁-C₆ heteroalkyl, or C₃-C₆cycloalkyl, wherein each of the alkyl, heteroalkyl, and cycloalkyl is optionally substituted.

In some embodiments, the metal chelator in Formula (X) is selected from AAZTA, BAT, BAT-TM, Crown, Cyclen, DO2A, CB-DO2A, DO3A, H₃HP-DO3A, Oxo-DO3A, p-NH₂-Bn-Oxo-DO3A, DOTA, DOTA-3py, DOTA-PA, DOTA-GA, DOTA-AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, IB4M-DTPA, p-SCN-BniB-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H3L′, H3L⁴, Hzazapa, H₅decapa, bispa², H₄pypa, H₄octapa, H₄CHXoctapa, p-SCN-Bn-H₄octapa, p-SCN-Bn-H₄soctapa, TTHA, p-NOz-Bn-neunpa, H₄octox, Hzmacropa, H₂bispa², H₄phospa, H₆phospa, p-SCN-Bn-Hhphospa, TETA, p-NOrBn-TETA, TRAP, TPA, HBED, SHBED, HBED-CC, (HBED-CC)TFP, DMSA, DMPS, DHLA, lipoic acid, TGA, BAL, Bis-thioseminarabazones, p-SCN-NOTA, nNOTA, NODAGA, CB-TE1AlP, 3P-C-NETA-NCS, 3p-C-DEPA, 3P-C-DEPA-NCS, TCMC, PCTA, NODIA-Me, TACN, pycuplAiB, pycup2A, THP, DEDPA, H DEDPA, p-SCN-Bn-H₂DEDPA, p-SCN-Bn-TCMC, motexafin, NTA, NOC, 3p-C-NETA, p-NHrBn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, L, L¹, L², L³, and EuK-106. In some embodiments, the metal chelator is 2,2′,2″,2′″-((2S,5S, 8S, 11S)-2,5,8,11-tetramethyl-1,4,7,10-tetraazacyclododecane-1,4,7,10-tetratetraacetic acid. In some embodiments, the metal chelator is 2,2′,2″,2′″-((2S,5S, 8S, 11S)-2,5,8,11-tetraethyl-1,4,7,10-tetraazacyclododecane-1,4,7,10-tetrayl)tetraacetic acid. In some embodiments, the metal chelator is a chelator in FIG. 1 to FIG. 15. In some embodiments, the metal chelator is DOTA. In some embodiments, the radionuclide is ⁶²Cu, ⁶³Cu, ⁶⁴Cu, ⁶⁸Ga, ⁸⁹Zr, ⁹⁹Y, ^{99m}Tc, ¹⁰⁵Rh, ¹¹¹In, ¹³⁴Ce, ¹⁴⁸Gd, ¹⁴⁹Tb, ¹⁵²Tb, ¹⁵³Pm, ¹⁶⁷Tm, ¹⁷⁵Yb, ¹⁷⁷Lu, ²⁰⁹Bi, ²¹²Pb, ²¹³Po, ²¹³Bi, ²²³Ra, ²²³Fr, ²²⁷Th, ²²⁵Ac, or ²²⁹Th.

In one aspect, provided herein is a pharmaceutical composition comprising a conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVc), Formula (X), Formula (X-III), or Formula (X-IV) and a pharmaceutically acceptable excipient or carrier. In some embodiments, the pharmaceutical composition is formulated for intravenous administration.

In another aspect, provided herein are methods of making a covalently modified KRAS G12C protein in vivo comprising administering a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula (X), Formula (X-III), or Formula (X-IV) or salt or solvate or pharmaceutical composition thereof to a subject. In some embodiments, the subject has a KRAS protein comprising a glycine to cysteine amino acid substitution at residue 12. In some embodiments, the subject has a cancer

In one aspect, provided herein are methods of treating cancer in a subject in need thereof, comprising administering to the subject a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula (X), Formula (X-III), or Formula (X-IV) or a salt or solvate or pharmaceutical composition thereof. In some embodiments, the cancer is selected from the group consisting of Cardiac cancer; sarcoma (angiosarcoma, fibrosarcoma, rhabdomyosarcoma, liposarcoma), myxoma, rhabdomyoma, fibroma, lipoma and teratoma; Lung cancer: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal cancer: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract cancer: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver cancer: hepatoma (hepatocellular carcinoma), cholangiocarcinoma, hepatoblastoma, angiosarcoma, hepatocellular adenoma, hemangioma; Biliary tract cancer: gall bladder carcinoma, ampullary carcinoma, cholangiocarcinoma: Bone cancer: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors: Nervous system cancer: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological cancer: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic cancer: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin cancer: malignant melanoma, basal cell carcinoma, squamous cell carcinoma, Kaposi's sarcoma, moles dysplastic nevi, lipoma, angioma, dermatofibroma, keloids, psoriasis; and Adrenal glands cancer: neuroblastoma. In some embodiments, the cancer is non-small cell lung cancer.

In one aspect, provided herein is a method of killing a cell harboring a G12C KRAS mutation, the method comprising contacting a cell harboring a G12C KRAS mutation with the conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula W, Formula (X-III), or Formula (X-IV), or a pharmaceutical composition comprising the conjugate, thereby delivering a dose of radiation to the cell.

In one aspect, provided herein is a method of delivering a radionuclide to a cell comprising administering the conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula (X), Formula (X-III), or Formula (X-IV), or a pharmaceutical composition comprising the conjugate. In some embodiments, the conjugate irreversibly binds to an intracellular protein of the cell. In some embodiments the intracellular protein is G12C KRAS.

In one aspect, provided herein is a method of diagnosing cancer patients harboring a G12C KRAS mutation comprising administering to a patient the conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula (X), Formula (X-III), or Formula (X-IV), or a pharmaceutical composition comprising the conjugate. In some embodiments, the method of diagnosing cancer patients harboring a G12C KRAS mutation further comprises measuring the concentration of the conjugate accumulated in the patient. In some embodiments, the method of diagnosing cancer patients harboring a G12C KRAS mutation further comprises measuring the amount of radiation emitted from the radionuclide. In some embodiments, the method of diagnosing cancer patients harboring a G12C KRAS mutation further comprises analyzing the elimination profile of the conjugate in the patient. In some embodiments, the method of diagnosing cancer patients harboring a G12C KRAS mutation further comprises measuring the elimination half-life of the conjugate in the patient.

In one aspect, provided herein is a method of imaging a cancer harboring a G12C KRAS mutation comprising administering to a patient the conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula (X), Formula (X-III), or Formula (X-IV), or a pharmaceutical composition comprising the conjugate. In some embodiments, the method of imaging cancer harboring a G12C KRAS mutation further comprises measuring the concentration of the conjugate accumulated in the patient. In some embodiments, the method of method of imaging a cancer harboring a G12C KRAS mutation further comprises measuring the amount of radiation emitted from the radionuclide. In some embodiments, the method of imaging a cancer harboring a G12C KRAS mutation further comprises analyzing the elimination profile of the conjugate in the patient. In some embodiments, the method of imaging a cancer harboring a G12C KRAS mutation further comprises measuring the elimination half-life of the conjugate in the patient.

In one aspect, provided herein is a method of treating cancer in a subject comprising administering
(i) a first radiopharmaceutical conjugate comprising a radionuclide configured for companion diagnostic and
(ii) a second radiopharmaceutical conjugate comprising a radionuclide selected from an alpha or beta-particle emitter, wherein the first and the second radiopharmaceutical conjugates have the same structure except for the radionuclide. In some embodiments, the radionuclide of the first radiopharmaceutical conjugate is selected from ⁶²Cu, ⁶⁴Cu, ⁸⁹Zr, ¹³⁴Ce, ¹⁵²Tb, ⁶⁸Ga, ¹¹¹In, and ^{99m}Tc. In some embodiments, the radionuclide of the second radiopharmaceutical conjugate is selected from ²²⁵Ac, ²¹³Bi, ²⁰⁹Bi, ¹⁴⁹Tb, ²²⁵Ra, ²²⁷Th, ²²³Fr, ¹⁴⁸Gd, ²⁹⁹Th ²¹³Po, ⁶⁷Cu, ¹⁷⁷Lu, ⁹⁰Y, ²¹²Pb, ¹⁰⁵Rh, ¹⁷⁵Yb, ¹⁶⁷Tm, ¹⁵³Pm, and ¹¹¹In. In some embodiments, the radionuclide of the first radiopharmaceutical conjugate is selected from ¹³C, ¹³N, ¹⁵O, ¹⁸F, ⁷⁴As, ⁷⁶Br, ¹²³I, ¹²⁴I and ¹²⁵I. In some embodiments, the radionuclide of the second radiopharmaceutical conjugate is selected from ¹³¹I and ²¹¹At.

In one aspect, provided herein are methods of producing a compound a structure of Formula (VIa), Formula (VIb), Formula (VIc), or Formula (VId) in vivo, comprising administering a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe) or salt or solvate or pharmaceutical composition thereof to a subject,

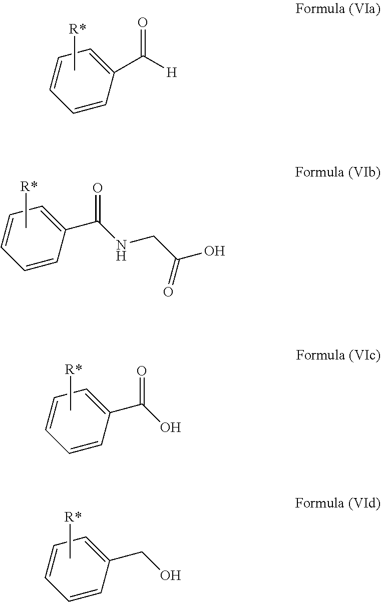

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At).

In one aspect, provided herein are methods of excreting a compound having a structure of Formula (VIa), Formula (VIb), Formula (VIc), or Formula (VId) in -vivo, comprising administering a radiolabeled compound of Formula (III), Formula (IIIa-1), Formula (IIIa-2), Formula (IIIa), Formula (IIIb), Formula (IIIc), Formula (IV), Formula (IVa), Formula (IVb), Formula (IVc), Formula (IVd), or Formula (IVe) or salt or solvate or pharmaceutical composition thereof to a subject.

In one aspect, provided herein are methods of making the modified KRAS proteins and compounds of the present application, such as a compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), or Formula (X).

Any combination of the groups described above for the various variables is contemplated herein. Throughout the specification, groups and substituents thereof are chosen by one skilled in the field to provide stable moieties and compounds.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1-FIG. 15 illustrate the structures of representative metal chelators.

FIGS. 16A to 16B illustrates exemplary radiolabeled compounds of the present disclosure forming a covalent bond with KRAS G12C. FIG. 16A illustrates a radiolabeled compound comprising a radioisotope R*, where R* falls outside of the KRAS G12C binding pocket. FIG. 16B illustrates a radiolabeled compound comprising a radioisotope R*, where R* is within the KRAS G12C binding pocket.

### DETAILED DESCRIPTION

Described herein are compositions of targeted radiotherapies (TRT) and methods of making and using the same. In the case of oncology, the TRT construct can comprise a high affinity ligand that specifically binds to a tumor associated target, for example, KRAS and KRAS mutants. The high affinity ligand may be a small molecule, an antibody, etc, and can be designed to deliver a dose of radiation directly to the tumor target.

In one aspect, the present disclosure describes a TRT that covalently modifies a KRAS protein with a radiolabeled compound comprising a covalently bonded radioisotope. In another aspect, the present disclosure describes a TRT that covalently modifies a KRAS protein with a radiolabeled compound comprising a chelator configured to bind a radionuclide. These unique TRTs have broad applications in the field of oncology therapy and diagnostics.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the an. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

As used in the specification and appended claims, unless specified to the contrary, the following terms have the meaning indicated below.

As used herein and in the appended claims, the singular forms “a.” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “comprising” (and related terms such as “comprise” or “comprises” or “having” or “including”) are to be construed in an open, inclusive sense, that is, as “including, but not limited to.” The term “comprising” (and related terms such as “comprise” or “comprises” or “having” or “including”) is not intended to exclude that in other certain embodiments, for example, an embodiment of any composition of matter, composition, method, or process, or the like, described herein. “consist of” or “consist essentially of” the described features.

“Amino” refers to the —NH₂ radical.

“Cyano” refers to the —CN radical.

“Nitro” refers to the —NO₂ radical.

“Oxo” refers to the ═O radical.

“Hydroxy” or “hydroxyl” refers to the —OH radical.

“Hydroxyalkyl” refers to an alkyl as defined below substituted with one or more hydroxy radicals. In some embodiments, the alkyl is substituted with 1, 2, 3, or 4 hydroxyl radicals. In some embodiments, the alkyl is substituted with 4 hydroxyl radicals. In some embodiments, the alkyl is substituted with 3 hydroxyl radicals, in some embodiments, the alkyl is substituted with 2 hydroxyl radicals. In some embodiments, the alkyl is substituted with 1 hydroxyl radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl (or iPr), 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C═CH. In some embodiments, the alkyl is optionally substituted with oxo, halogen, —CN, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN. —CF₃, —OH, —OMe, —NH₂, or —NO₂. In some embodiments, an alkylene is optionally substituted with oxo, halogen, —CN, —CF₃. —OH, or —OMe. In some embodiments, the alkylene is optionally substituted with halogen. In some embodiments, the alkylene is —CH₂—, —CH₂CH₂—, —CH₂CH₂CH₂—, or —CH₂CH(CH₃)CH₂—. In some embodiments, the alkylene is —CH₂—. In some embodiments, the alkylene is —CH₂CH₂—. In some embodiments, the alkylene is —CH₂CH₂CH₂—.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (—CH═CH₂), 1-propenyl (—CH₂CH═CH₂), isopropenyl (—C(CH₃)═CH₂—, butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₁-C₄ alkenyl, a C₁-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

The term “alkenylene” or “alkenylene chain” refers to an optionally substituted straight or branched divalent hydrocarbon chain in which at least one carbon-carbon double bond is present linking the rest of the molecule to a radical group. In some embodiments, the alkenylene is —CH═CH—, —CH₂CH═CH₂, or —CH═CHCH₂—. In some embodiments, the alkenylene is —CH═CH—. In some embodiments, the alkenylene is —CH₂CH═CH—. In some embodiments, the alkenylene is —CH═CHCH₂—.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an all vinyl is optionally substituted with oxo, halogen, —CN, —CF₃, —OH, —OMe, —NH—, or —NO—. In some embodiments, an alkynyl is optionally substituted with oxo, halogen, —CN, —CF₃, —OH, or —OMe. In some embodiments, the alkynyl is optionally substituted with halogen. The term “alkynylene” refers to an optionally substituted straight-chain or optionally substituted branched-chain divalent hydrocarbon having one or more carbon-carbon triple-bonds.

“Alkylamino” refers to a radical of the formula —N(R)₂ where Rₐ is an alkyl radical as defined, or two Rₐ, taken together with the nitrogen atom, can form a substituted or unsubstituted C₂-C₇ heterocyloalkyl ring. Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, —OH, —OMe, —NH₂, or —NO₂. In some embodiments, an alkylamino is optionally substituted with oxo, halogen, —CN, —CF₃, —OH, or —OMe. In some embodiments, the alkylamino is optionally substituted with halogen, Alkyl groups, as defined above, may be optionally substituted with an alkylamino group (e.g., an alkylaminylalkyl or dialkylaminylalkyl).

“Alkoxy” or “alkoxyl” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

An alkoxy substituted with one or more halogen is referred to herein as “haloalkoxy”. In some embodiments, the alkoxy is substituted with one, two, or three halogens. In some embodiments, the alkoxy is substituted with one, two, three, four, five, or six halogens. Haloalkoxy can include, for example, iodoalkoxy, bromoalkoxy chloroalkoxy, and fluoroalkoxy. For example, “fluoroalkoxy” refers to an alkoxy radical, as defined above, that is substituted by one or more fluoro radicals.

“Alkylthio”, “alkylsulfoxide”, and “alkylsulfone” refer to a radical of the formula —SR₂, —S(O)Rₐ, or —S(O)₂Rₐ, respectively, where Rₐ is an alkyl radical as defined. Unless stated otherwise specifically in the specification, an alkylthio, alkylsulfoxide, or alkylsulfone group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylthio, alkylsulfoxide, or alkylsulfone is optionally substituted with oxo, halogen, —CN, —CF₃, —OH, —OMe, —NH₂, or —NO₂. In some embodiments, an alkylthio, alkylsulfoxide, or alkylsulfone is optionally substituted with oxo, halogen, —CN, —CF₃, —OH, or —OMe. In some embodiments, the alkylthio, alkylsulfoxide, or alkylsulfone is optionally substituted with halogen.

“Alkyloxy” refers to an alkyl group in which one or more skeletal atoms of the alkyl are replaced with oxygen. Unless stated otherwise specifically in the specification, an alkyloxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkyloxy is optionally substituted with oxo, halogen, —CN, —CF₃, —OH, —OMe, —NH₂, or —NO₂. In some embodiments, an alkyloxy is optionally substituted with oxo, halogen, —CN, —CF₃, —OH, or —OMe. In some embodiments, the alkyloxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be monovalent or divalent (i.e., an arylene group). Unless stated otherwise specifically in the specification, the teen “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH₂CH₃, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

“Aryloxy” refers to an aryl group as defined above connected to the rest of the molecule through —O—.

“Arylthio” refers to an aryl group as defined above connected to the rest of the molecule through —S—.

“Arylsulfoxide” refers to an aryl group as defined above connected to the rest of the molecule through —S(O)—.

“Arylsulfone” refers to an aryl group as defined above connected to the rest of the molecule through —S(O)₂—.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Depending on the structure, a cycloalkyl group can be monovalent or divalent (i.e., a cycloalkylene group). Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₆ cycloalkyl), or three to four carbon atoms (C₃-C₄ cycloalkyl). A cycloalkyl can comprise a fused, spiro or bridged ring system. In some embodiments, the cycloalkyl comprises a fused ring system. In some embodiments, the cycloalkyl comprises a spiro ring system. In some embodiments, the cycloalkyl comprises a bridged ring system. In some embodiments, the cycloalkyl comprises an alkene (e.g., a cycloalkenyl). In some embodiments, the cycloalkyl comprises an alkyne (e.g., a cycloalkynyl). In some embodiments, the cycloalkyl is a 3- to 6-membered cycloalkyl. In some embodiments, the cycloalkyl is a 5-to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro. In some embodiments, halogen is a radionuclide selected from fluorine-18 (¹⁸F), iodine-131 (¹³¹I), iodine-123 (¹²³I), iodine-124 (¹²⁴I), iodine-125 (¹²⁵I), and astatine-211 (²¹¹At).

A “radiolabeled conjugate” or “radiolabeled compound” is used herein interchangeably to refer to a compound comprising a radionuclide. In some embodiments, a radiolabeled compound comprises a covalently attached radionuclide. In some embodiments, a radiolabeled compound comprises a non-covalently attached radionuclide. In some embodiments, the radionuclide is attached via a metal chelator.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl. 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂—O—CH₃, —CH₂—N(alkyl)-CH₃, —CH₂—N(aryl)-CH₃, —OCH₂CH₂OH, —OCH₂CH₂OCH₂CH₂OH, or —OCH₂CH₂OCH₂CH₂OCH₂CH₂OH. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. As used herein, a “heteroalkylene” refers to divalent heteroalkyl group. Examples of such heteroalkylene are, for example, —CH₂—O—CH₂—, —CH₂—N(alkyl)-CH₂—, —CH₂—N(aryl)-CH₂—, —OCH₂CH₂O—. —OCH₂CH₂OCH₂CH₂O—, or —OCH₂CH₂OCH—CH—OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. A heterocycloalkyl can comprise a fused, spiro or bridged ring system. In some embodiments, the heterocycloalkyl comprises a fused ring system. In some embodiments, the heterocycloalkyl comprises a spiro ring system. In some embodiments, the heterocycloalkyl comprises a bridged ring system. Depending on the structure, a heterocycloalkyl group can be monovalent or divalent (i.e., a heterocycloalkylene group). Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a ring system radical comprising carbon atom(s) and one or more ring heteroatoms that selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, Spiro or bridged ring systems. Depending on the structure, a heteroaryl group may be monovalent or divalent (e.g., a heteroarylene group). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein. “treatment,” “prevention,” “amelioration,” or “inhibition” encompass delaying the onset of the disorder, or a symptom or condition thereof. As used herein. “treating” includes the concepts of “alleviating”, which refers to lessening the frequency of occurrence or recurrence, or the severity, of any symptoms or other ill effects related to a disorder and/or the associated side effects. The teen “treating” also encompasses the concept of “managing” which refers to reducing the severity of a particular disease or disorder in a patient or delaying its recurrence. e.g., lengthening the period of remission in a patient who had suffered from the disease.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

The term “optionally substituted” or “substituted” means that the referenced group is optionally substituted with one or more additional group(s) individually and independently selected from D, halogen, —CN, oxo, —NH₂, —NH(alkyl), —N(alkyl)₂, —OH, —CO₂H, —CO₂alkyl, —C(O)NH₂, —C(═O)NH(alkyl). —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, oxo, —NH₂, —NH(CH₃), —N(CH₂)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₆alkyl)₂, C₁-C₄alkyl, C₃-C₆cycloalkyl, C₁-C₁fluoroalkyl, C₁-C₄heteroalkyl, C₁-C₄alkoxy, C₁-C₄fluoroalkoxy, —SC₁-C₄alkyl, —S(O)C₁-C₄alkyl, and —S(═O)₂C₁-C₄alkyl. In some embodiments, optional substituents are independently selected from D, halogen, —CN, —NH₂, —OH. —NH(CH₂), —N(CH₃)₂, —NH(cyclopropyl), —CH₃. —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). When indicating the number of substituents, the term “one or more” means from one substituent to the highest possible number of substitution, i.e. replacement of one hydrogen up to replacement of all hydrogens by substituents.

The term “unsubstituted” means that the specified group bears no substituents.

Certain compounds described herein may exist in tautomeric forms, and all such tautomeric forms of the compounds being within the scope of the disclosure.

Unless otherwise stated, structures depicted herein are also meant to include all stereochemical forms of the structure: i.e., the R and S configurations for each asymmetric center. Therefore, single stereochemical isomers as well as enantiomeric and diastereomeric mixtures of the present compounds are within the scope of the disclosure.

The term “protein” as used herein refers to a polypeptide (i.e., a string of at least 3 amino acids linked to one another by peptide bonds). Proteins can include moieties other than amino acids (e.g., may be glycoproteins, proteoglycans, etc.) and/or can be otherwise processed or modified. A protein can be a complete polypeptide as produced by and/or active in a cell (with or without a signal sequence). In some embodiments, a protein is or comprises a characteristic portion such as a polypeptide as produced by and/or active in a cell. A protein can include more than one polypeptide chain.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges. “nested sub-ranges” that extend from either end point of the range are specifically contemplated. For example, a nested sub-range of an exemplary range of to 50 may comprise 1 to 10, 1 to 20, 1 to 30, and 1 to 44) in one direction, or 50 to 40, 50 to 30, 50 to 20, and 50 to 10 in the other direction.

As used herein, C₁-Cₓ, (or C₁₋ₓ) includes C₁-C₂, C₁-C₃ . . . C₁-Cₓ. By way of example only, a group designated as “C₁-C₄” indicates that there are one to four carbon atoms in the moiety. i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂, and —CH₂CH₂ linkages.

The term “subject” or “patient” encompasses mammals. Examples of mammals include, but are not limited to, any member of the Mammalian class: humans, non-human primates such as chimpanzees, and other apes and monkey species: farm animals such as cattle, horses, sheep, goats, swine: domestic animals such as rabbits, dogs, and cats: laboratory animals including rodents, such as rats, mice and guinea pigs, and the like. In one aspect, the mammal is a companion animal such as a dog or a cat. In one aspect, the mammal is a human.

### II. Covalently Modified KRAS Protein and Covalent Binders

In one aspect, described herein is a radiopharmaceutical compound comprising a) a targeting ligand that covalently binds to a mutated KRAS protein (such as KRAS G12C): b) a radioisotope connected to the targeting ligand, either covalently or via a metal chelator, and optionally, c) a linker covalently connecting the radioisotope or the metal chelator to the targeting ligand. In one aspect, described herein are compounds comprising a) a targeting ligand that forms a covalent bond with KRAS G12C based on SEQ ID NO: 1 or SEQ ID NO: 2, b) a covalent radioisotope or a metal chelator configured to bind a radioisotope, and optionally, c) a linker covalently connecting the radioisotope or metal chelator to the targeting ligand. In some embodiments, the targeting ligand comprises a structure selected from Table 1. In some embodiments, the targeting ligand comprises a derivative, or a binding fragment of the structures in Table 1.

TABLE 1
Exemplary Targeting Ligand (attachment point to the
conjugate not shown).

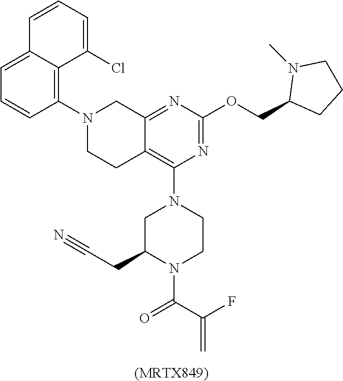

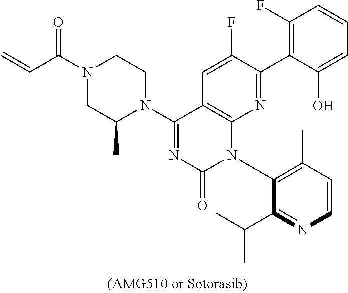

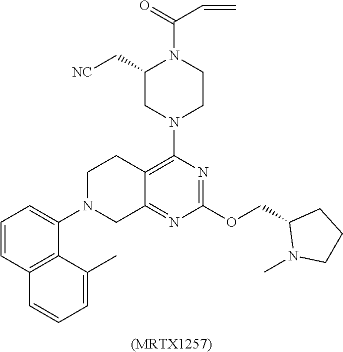

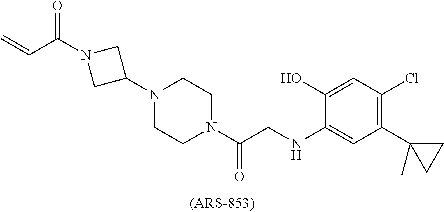 [SMILES: C=CC(=O)N1CC(N2CCN(C(=O)CNc3cc(C4(C)CC4)c(Cl)cc3O)CC2)C1]

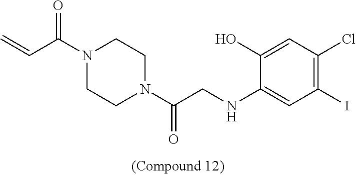 [SMILES: C=CC(=O)N1CCN(C(=O)CNc2cc(I)c(Cl)cc2O)CC1]

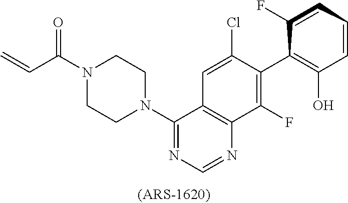

In one aspect, described herein are modified KRAS proteins comprising a covalently and irreversibly bound radiolabeled compound, wherein the radiolabeled compound comprises a covalently bonded radioisotope. In some embodiments, the modified KRAS protein comprises one or more amino acid mutations. In some embodiments, the modified KRAS protein comprises a G12C mutation based on SEQ ID NO: 1 or SEQ ID NO: 2. In one aspect, described herein are compounds or conjugates designed to covalently and irreversibly bind to an intracellular mutated GTPase KRas (KRAS) protein. In some embodiments, the intracellular mutated protein is encoded by a KRAS gene. In some embodiments, the mutation of the KRAS protein comprises a G12C mutation based on SEQ ID No: 1. In some embodiments, the mutation of the KRAS protein comprises a G12C mutation based on SEQ ID No: 2. In some embodiments, the electrophilic functional group of the radiolabeled compound or conjugate covalently binds to the intracellular mutated protein at a cysteine residue. The radiolabeled compound or conjugate can bind to a mutant-specific cysteine residue of the mutated KRAS protein. The radiolabeled compound or conjugate can form a covalent bond with a mutant-specific cysteine residue of the mutated KRAS protein. The mutant-specific cysteine residue of the mutated KRAS protein can be G12C. In some embodiments, the covalently modified KRAS protein comprises a radiopharmaceutical conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula (X), Formula (X-III), or Formula (X-IV). In some embodiments, the covalently modified KRAS protein comprises a radiopharmaceutical conjugate of Formula (III), Formula (IIIa), Formula (IIIa-1), Formula (IIIa-2), Formula (IIIb), or Formula (IIIc). In some embodiments, the covalently modified KRAS protein comprises a radiopharmaceutical conjugate of Formula (IV), Formula (IVa), Formula (IVb), Formula (IVc), Formula (IVd), or Formula (IVe). In some embodiments, the covalently modified KRAS protein comprises a radiopharmaceutical conjugate of Formula (X), Formula (X-ITT), or Formula (X-IV). In some embodiments, the covalently modified KRAS protein comprises a radiopharmaceutical conjugate of Formula (III). In some embodiments, the covalently modified KRAS protein comprises a radiopharmaceutical conjugate of Formula (IIIa). In some embodiments, the covalently modified KRAS protein comprises a radiopharmaceutical conjugate of Formula (IIIa-1). In some embodiments, the covalently modified KRAS protein comprises a radiopharmaceutical conjugate of Formula (IIIa-2). In some embodiments, the covalently modified KRAS protein comprises a radiopharmaceutical conjugate of Formula (IIIb). In some embodiments, the covalently modified KRAS protein comprises a radiopharmaceutical conjugate of Formula (IIIc). In some embodiments, the covalently modified KRAS protein comprises a radiopharmaceutical conjugate of Formula (IV). In some embodiments, the covalently modified KRAS protein comprises a radiopharmaceutical conjugate of Formula (IVa). In some embodiments, the covalently modified KRAS protein comprises a radiopharmaceutical conjugate of Formula (IVb). In some embodiments, the covalently modified KRAS protein comprises a radiopharmaceutical conjugate of Formula (IVc), in some embodiments, the covalently modified KRAS protein comprises a radiopharmaceutical conjugate of Formula (IVd). In some embodiments, the covalently modified KRAS protein comprises a radiopharmaceutical conjugate of Formula (IVe). In some embodiments, the covalently modified KRAS protein comprises a radiopharmaceutical conjugate of Formula (X). In some embodiments, the covalently modified KRAS protein comprises a radiopharmaceutical conjugate of Formula (X-III). In some embodiments, the covalently modified KRAS protein comprises a radiopharmaceutical conjugate of Formula (X-IV).

TABLE 2
Exemplary KRAS Protein Wild-type Sequence

| | | SEQ | |
|---|---|---|---|
| Gene | Protein | ID NO | Amino Acid Sequence |
| KRAS | GTPase | 1 | MTEYKLVVVGAGGVGKSALTIQLIQN |
| | KRAS | | HFVDEYDPTIEDSYRKQVVIDGETCL |
| | | | LDILDTAGQEEYSAMRDQYMRTGEGF |
| | | | LCVFAINNTKSFEDIHHYREQIKRVK |
| | | | DSEDVPMVLVGNKCDLPSRTVDTKQA |
| | | | QDLARSYGIPFIETSAKTRQRVEDAF |
| | | | YTLVREIRQYRLKKISKEEKTPGCVK |
| | | | IKKCIIM |
| KRAS | GTPase | 2 | MTEYKLVVVGAGGVGKSALTIQLIQN |
| | KRAS | | HFVDEYDPTIEDSYRKQVVIDGETCL |
| | | | LDILDTAGQEEYSAMRDQYMRTGEGF |
| | | | LCVFAINNTKSFEDIHHYREQIKRVK |
| | | | DSEDVPMVLVGNKCDLPSRTVDTKQA |
| | | | QDLARSYGIPFIETSAKTRQGVDDAF |
| | | | YTLVREIRKHKEKMSKDGKKKKKKSK |
| | | | TKCVIM |

In some embodiments, radiolabeled compounds described herein bind to the GDP-bound form of KRAS G12C. In some embodiments, radiolabeled compounds described herein stabilize the switch-II loop of KRAS G12C.

In some embodiments, the radiolabeled compounds are radiolabeled with a covalently bonded radioisotope. In some embodiments, the radiolabeled compounds release a number of alpha particles, beta particles, gamma rays, and/or Auger electrons by natural radioactive decay. In some embodiments, the radiolabeled compound is covalently labeled with a radioisotope selected from fluorine-18 (¹⁸F) iodine-131 (¹³¹I), iodine-123 (¹²³I), iodine-124 (¹²⁴I), iodine-125 (¹²⁵I), or astatine-211 (²¹¹At). In some embodiments, the radiolabeled compound releases beta particles. In some embodiments, the radiolabeled compound releases gamma rays. In some embodiments, the radiolabeled compound releases Auger electrons. In some embodiments, the radiolabeled compound emits beta particles and the covalently bonded radioisotope is ¹³¹I. In some embodiments, the radiolabeled compound emits beta particles and the covalently bonded radioisotope is ¹²⁴I. In some embodiments, the radiolabeled compound emits Auger electrons and the covalently bonded radioisotope is ¹²¹I. In some embodiments, the radiolabeled compound emits alpha particles and the covalently bonded radioisotope is ²¹¹At.

In some embodiments, the KRAS protein is covalently modified by a radiolabeled compound comprising a covalently bonded radioisotope. In some embodiments, the covalent bond between the KRAS protein and the radiolabeled compound comprising a covalently bonded radioisotope is formed in vivo. In some embodiments, the KRAS protein comprises a glycine to cysteine amino acid substitution or mutation. In some embodiments, the glycine to cysteine amino acid substitution or mutation takes place at residue 12 (G12C) based on SEQ ID NO: 1 or SEQ ID NO: 2. In some embodiments, the KRAS protein comprising the glycine to cysteine substitution or mutation at residue 12 is covalently bonded to the radioisotope through the radiolabeled compound.

### Electrophilic Functional Groups

Described herein are compounds (such as radiolabeled compounds or conjugates) that comprise an electrophilic functional group and modified KRAS proteins with the compound bound thereto. In some embodiments, the radiolabeled compound or conjugate covalently bound to KRAS G12C comprises an electrophilic functional group. In some embodiments, the electrophilic functional group is reactive with a cysteine residue. In some embodiments, the electrophilic functional group is reactive with a cysteine residue in vivio. In some embodiments, the cysteine residue is KRAS G12C. In some embodiments, the covalent bond between the KRAS G12C protein and the radiolabeled compound or conjugate is formed between the electrophilic group of the radiolabeled compound or conjugate and the cysteine residue 12 of the KRAS protein. A person skilled in the art would appreciate that the electrophilic functional group can react with a KRAS protein, thereby forming a covalent bond between the compound or conjugate comprising the electrophilic functional group and the KRAS protein, resulting in a modified KRAS protein. Unless stated otherwise, electrophilic functional groups described herein are illustrated in a unreacted form.

In some embodiments, provided herein is a radiolabeled compound or conjugate comprising an electrophilic functional group. In some embodiments, provided herein is a modified KRAS protein with the radiolabeled compound or conjugate bound thereto via a covalent bond. In some embodiments, the electrophilic functional group comprises an ester, acrylamide, halo-acrylamide, acyl azide, acyl nitrite, aldehyde, ketone, alkyl halide, alkyl sulfonate, anhydride, aryl halides, boronic acid, boronate, carboxylic acid, hydrazide, carbamate, carbodiimide, diazoalkane, epoxide, haloacetamide, halotriazine, imido ester, isocyanate, isothiocyanate, maleimide, phosphoramidite, silyl halide, sulfonate ester, sulfonyl halide, α,β-unsaturated thione, α,β-unsaturated carbonyl, α-ketoamide, vinyl sulfone, vinyl amide, vinyl arylene, sulfonamide, propargyl amide group, propargyl ketone group, each of which is optionally substituted. In some embodiments, the electrophilic functional group comprises a 2-fluoroacrylamide group, or a 2-methyl acrylamide group. In some embodiments, the electrophilic functional group comprises a substituted enamide group comprising acrylamide, 2-fluoroacrylamide, methacrylamide, 2-methoxyacrylamide. (E)-4-fluorobut-2-enamide, (E)-4-methoxybut-2-enamide. (E)-4-(pyrrolidin-1-yl)but-2-enamide, or (E)-4-(piperidin-1-yl)but-2-enamide. In some embodiments, the electrophilic functional group covalently binds an amino acid residue. In some embodiments, the electrophilic functional group covalently binds a cysteine residue. In some embodiments, the electrophilic functional group covalently binds a G12C amino acid residue on a KRAS protein.

In some embodiments, an exemplary vinyl arylene can be

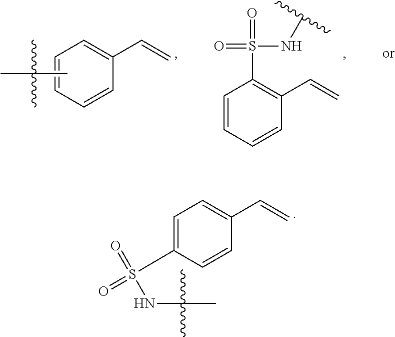

In some embodiments, an electrophilic functional group described herein comprises a substituted or unsubstituted acrylamide group. In some embodiments, the electrophilic functional group comprises a substituted acrylamide. In some embodiments, the electrophilic functional group comprises an unsubstituted acrylamide (or

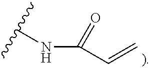 [SMILES: *NC(=O)C=C]

In some embodiments, the acrylamide is substituted with one or more substituents selected from halogen, alkoxy, amino, OH, CN, C₁₋₆ alkyl, C₁₋₆ heteroalkyl, C₃₋₆ cycloalkyl, and C₂₋₆ heterocycloalkyl. In some embodiments, the electrophilic functional group comprises halo-acrylamide. In some embodiments, the substituted acrylamide is 2-fluoroacrylamide, 2-chloroacrylamide, or a derivative thereof. In some embodiments, the electrophilic functional group comprises 2-fluoroacrylamide. In some embodiments, the electrophilic functional group comprises an α,β-unsaturated carbonyl. In some embodiments, the α,β-unsaturated carbonyl comprises an α,β-unsaturated ketone, α,β-unsaturated aldehyde, α, β-unsaturated amide, α,β-unsaturated acid, or α,β-unsaturated ester, each of which is optionally substituted.

In some embodiments, an electrophilic functional group described herein comprises a substituted or unsubstituted chloroacetamide group. In some embodiments, the electrophilic functional group comprises an unsubstituted chloroacetamide group (or

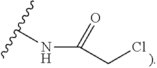 [SMILES: *NC(=O)CCl]

In some embodiments, the electrophilic functional group comprises a substituted or unsubstituted acyl azide group. In some embodiments, the electrophilic functional group comprises a substituted or unsubstituted carbamate group. In some embodiments, the electrophilic functional group comprises a substituted or unsubstituted α,β-unsaturated carbonyl group. In some embodiments, the electrophilic functional group comprises a substituted or unsubstituted α-ketoamide group. In some embodiments, the electrophilic functional group comprises a substituted or unsubstituted propargyl amide group. In some embodiments, the electrophilic functional group comprises a substituted or unsubstituted propargyl ketone group.

In some embodiments, the substituted acrylamide is 2-fluorocryalamide, 2-chloroacrylamide, or a derivative thereof. In some embodiments, the electrophilic group is an α,β-unsaturated carbonyl. In some embodiments, the α,β-unsaturated carbonyl is an α,β-unsaturated ketone, α,β-unsaturated aldehyde, α,β-unsaturated amide, α,β-unsaturated acid, or α,β-unsaturated ester, each of which is optionally substituted.

In some embodiments, an electrophilic functional group described herein comprises an acceptor of Michael Addition. In some embodiments, a Michael acceptor comprises a functional group having a structure of

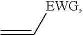

wherein EWG represents an electron withdrawing group. Exemplary Michael acceptors include

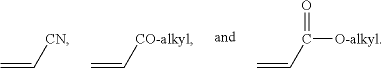

Exemplary Michael acceptors further include

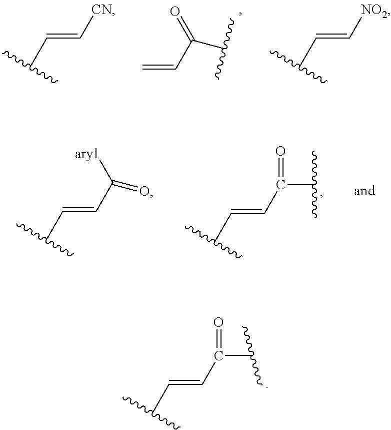

In some embodiments, provided herein are radiolabeled compounds comprising an electrophilic functional group of Formula (Ia):

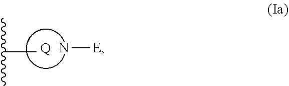

wherein,
ring Q is a 3 to 10 membered heterocycloalkylene, wherein Q is optionally substituted; and
E represents a structure of Formula (Ic).

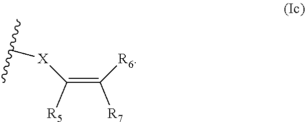

wherein,
X is C(═O), P(═O)OR², C(═S), or S(═O)ₙ, where n is 1 or 2;
R² is hydrogen or substituted or unsubstituted C₁-C₃ alkyl;
R⁵ and R⁷ are each independently selected from hydrogen, —CN, halogen, substituted or unsubstituted C₁-C₆ alkyl, substituted or unsubstituted C₁-C₆ heteroalkyl, substituted or unsubstituted C₃-C₇ cycloalkyl, or substituted or unsubstituted C₂-C₇ heterocycloalkyl, or R⁵ and R⁷ taken together form a bond; and
R⁶ is hydrogen, halogen, —CN, C₁-C₆ alkyl, substituted or unsubstituted C₁-C₆ heteroalkyl, substituted or unsubstituted heteroaryl, substituted or unsubstituted aryl, substituted or unsubstituted alkylaminylalkyl, substituted or unsubstituted dialkylaminylalkyl, substituted or unsubstituted cycloalkyl or substituted or unsubstituted heterocycloalkyl.

In some embodiments, ring Q of Formula (Ia) is a 3-membered, 4-membered, 5-membered, 6-membered, or 7-membered heterocycloalkylene ring with at least one nitrogen. In some embodiments of Formula (Ia), ring Q is substituted. In some embodiments, ring Q of Formula (Ia) is a diazetidine, azetidine, imidazolidine, pyrrolidine, piperidine, or piperazine ring. In some embodiments, ring Q of Formula (Ia) is a C₂-C₆ optionally substituted monocyclic heterocycloalkylene. In some embodiments, ring Q is 3-6 membered monocyclic heterocycloalkylene. In some embodiments, ring Q comprises 1 or 2 nitrogen atoms. In some embodiments, ring Q of Formula (Ia) is a C₅-C₉ optionally substituted bicyclic heterocycloalkylene. In some embodiments, ring Q is a spiro bicyclic heterocycloalkylene. In some embodiments, ring Q is a fused bicyclic heterocycloalkylene. In some embodiments, ring Q is a bridged bicyclic heterocycloalkylene.

In some embodiments, ring Q is optionally substituted with one or more R^{Q} groups, wherein each R^{Q} is independently D, halogen, —CN, —NH₂, —NH(alkyl), —N(alkyl)₂, —OH, oxo, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted. In some embodiments, ring Q is substituted with 1 R^{Q} group. In some embodiments, ring Q is substituted with 2 R^{Q} groups. In some embodiments, ring Q is substituted with 3 R^{Q} groups. In some embodiments, ring Q is substituted with 4 R^{Q} groups.

In some embodiments, ring Q is optionally substituted with one or more R^{Q} groups, wherein each R^{Q} is independently D, oxo, halogen, —CN, —NH₂, —OH, —NH(C₁-C₃alkyl), —N(C₁-C₃alkyl)₂, —NH(cyclopropyl), C₁-C₆alkyl, or C₁-C₆alkoxyl, wherein the alkyl or alkoxyl is optionally substituted with —CN and/or one or more halogens. In some embodiments, R^{Q} is alkyl substituted with —CN. In some embodiments, R^{Q} is alkyl substituted with one or more halogens. In some embodiments, R^{Q} is alkyl substituted with one, two, or three fluorine atoms.

In some embodiments, an electrophilic functional group described herein comprises a structure of Formula (Ib):

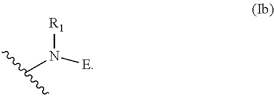

wherein,
R¹ is hydrogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₆ heterocycloalkyl; and
E comprises a structure of Formula (Ic),

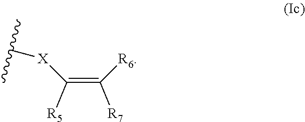

wherein,
X is C(═O), P(═O)OR², C(═S), or S(═O)ₙ;
n is 1 or 2;
R² is hydrogen or substituted or unsubstituted C₁-C₃ alkyl;
R⁵ and R⁷ are each independently selected from hydrogen, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₇ heterocycloalkyl; or R⁵ and R⁷ taken together form a bond; and
R⁶ is hydrogen, halogen, —CN, C₁-C₆ alkyl, substituted or unsubstituted C₁-C₆heteroalkyl, substituted or unsubstituted heteroaryl, substituted or unsubstituted aryl, substituted or unsubstituted alkylaminylalkyl, substituted or unsubstituted dialkylaminylalkyl, substituted or unsubstituted cycloalkyl or substituted or unsubstituted heterocycloalkyl.

In some embodiments of Formula (Ia) or Formula (Ib), X is C(═O), P(═O)OR², S(═O), or S(O)₂. In some embodiments of Formula (Ia) or Formula (Ib), X is C(═O). In some embodiments of Formula (Ia) or Formula (Ib), X is S(═O)₂. In some embodiments of Formula (Ia) or Formula (Ib), X is P(═O)OR². In some embodiments of Formula (Ib), R¹ is hydrogen, substituted or unsubstituted C₁-C₆ alkyl. In some embodiments of Formula (Ib), R¹ is hydrogen. In some embodiments of Formula (Ib), R¹ is methyl, ethyl, propyl, isopropyl, butyl, or tert-butyl.

In some embodiments, the electrophilic functional group has a structure of Formula (Ia). In some embodiments, the electrophilic functional group has a structure of Formula (Ib).

In some embodiments, an electrophilic functional group described herein comprises a structure of Formula (Id):

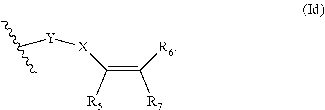

wherein,
X is C(═O), OC(═O), NR²C(═O), N(═NR²), NR²P(═O)OR², C(═S), N(═O), S(═O)ₙ, OS(O)ₙ, NR²S(═O), where n is 1 or 2;
Y is a bond, C₁-C₆ alkylene, C₁-C₆ heteroalkylene, cycloalkylene, heterocycloalkylene, arylene or heteroarylene, wherein each of the alkylene, heteroalkylene, cycloalkylene, heterocycloalkylene, arylene, or heteroarylene is optionally substituted;
each R² is independently hydrogen or substituted or unsubstituted C₁-C₃ alkyl;
R⁵ and R⁷ are each independently selected from hydrogen, —CN, halogen, substituted or unsubstituted C₁-C₆ alkyl, substituted or unsubstituted C₁-C₆heteroalkyl, substituted or unsubstituted C₃-C₆ cycloalkyl, or substituted or unsubstituted C₂-C₅ heterocycloalkyl; or R⁵ and R⁷ taken together form a bond; and
R⁶ is hydrogen, halogen, —CN, C₁-C₆ alkyl, C₁-C₆heteroalkyl, heteroaryl, aryl, —C₁-C₆alkylene-NH(C₁-C₆ alkyl), —C₁-C₆alkylene-N(C₁-C₆ alkyl)₂, C₃-C₆ cycloalkyl or C₂-C₅ heterocycloalkyl, wherein each of the alkyl, heteroalkyl, heteroaryl, aryl, cycloalkyl, and heterocycloalkyl are optionally substituted.

In some embodiments of Formula (Id), X is C(═O), OC(═O), NR²C(═O), P(═O)OR², C(═S), S(═O)ₙ, OS(O)ₙ, NR²S(═O)ₙ, wherein n is 1 or 2. In some embodiments, X of Formula (Id) is C(═O), OC(═O), NR²C(═O), N(═NR²), NR²P(═O)OR², C(═S), S(O), OS(O)ₙ, NR²S(═O)ₙ, where n is 1 or 2. In some embodiments of Formula (Id), X is C(═O). In some embodiments, X of Formula (Id) is C(═O). In some embodiments, X of Formula (Id) is NR²C(═O). In some embodiments, X of Formula (Id) is S(O). In some embodiments, X of Formula (Id) is S(═O)₂ In some embodiments of Formula (Id), X is NR²S(═O)ₙ, wherein n is 1 or 2.

In some embodiments of Formula (Id), Y is a bond, C₁-C₆ alkylene, C₁-C₆ heteroalkylene, cycloalkylene, heterocycloalkylene, arylene or heteroarylene, wherein each of the alkylene, heteroalkylene, cycloalkylene, heterocycloalkylene, arylene, or heteroarylene is optionally substituted. In some embodiments of Formula (Id). Y is a bond. In some embodiments of Formula (Id), Y is an alkylene.

In some embodiments, Y of Formula (Id) is substituted or unsubstituted C₁-C₄ alkylene, or substituted or unsubstituted C₁-C₄ heteroalkylene. In some embodiments, Y is an alkylene.

In some embodiments, Y of Formula (Id) is substituted or unsubstituted monocyclic arylene, or substituted or unsubstituted monocyclic heteroarylene. In some embodiments, Y is substituted or unsubstituted phenylene. In some embodiments of Formula (Id). Y is an alkylene or arylene.

In some embodiments, Y of Formula (Id) is substituted or unsubstituted 3 to 10 membered cycloalkylene, or substituted or unsubstituted 3 to 10 membered heterocycloalkylene. In some embodiments, Y of Formula (Id) is substituted or unsubstituted monocyclic or bicyclic cycloalkylene. In some embodiments. Y of Formula (Id) is substituted or unsubstituted monocyclic or bicyclic heterocycloalkylene. In some embodiments, Y is a 3-membered, 4-membered. S-membered, 6-membered, or 7-membered heterocycloalkylene ring with at least one nitrogen. In some embodiments, Y is substituted. In some embodiments, Y is a diazetidine, azetidine, imidazolidine, pyrrolidine, piperidine, or piperazine ring. In some embodiments, Y is a C₂-C₆ optionally substituted monocyclic heterocycloalkylene. In some embodiments, Y is 3-6 membered monocyclic heterocycloalkylene. In some embodiments, Y comprises 1 or 2 nitrogen. In some embodiments, Y is a C₅-C₉ optionally substituted bicyclic heterocycloalkylene. In some embodiments. Y is a spiro bicyclic heterocycloalkylene. In some embodiments, ring Q is a fused bicyclic heterocycloalkylene. In some embodiments, ring Q is a bridged bicyclic heterocycloalkylene.

In some embodiments, Y of Formula (Id) is optionally substituted with one or more R^{Q} groups, wherein each R^{Q} is independently D, halogen, —CN, —NH₂, —NH(alkyl), —N(alkyl)₂, —OH, oxo, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

In some embodiments, each R^{Q} of Formula (Ia), Formula (Ib) or Formula (Id) is independently oxo, hydroxy, —CN, halogen, C₁₋₆ alkyl, C₁₋₆alkenyl, C₁₋₆alkoxy, C₃₋₇cycloalkyl, C₁₋₆alkyl-OH, trihalo-C₁-C₆ alkyl, mono-C₁₋₆ alkylamino, di-C₁₋₆ alkylamino, —C(═O)NH₂, —NH₂, —NO₂, hydroxy-C₁₋₆ alkylamino, hydroxy-C₁₋₆ alkyl, 4-7 membered heterocycle-C₁₋₆ alkyl, amino-C₁₋₆ alkyl, mono-C₁₋₆ alkylamino-C₁₋₆ alkyl, and di-C₁₋₆ alkylamino-C₁₋₆ alkyl. In some embodiments, each R^{Q} of Formula (Ia), Formula (Ib) or Formula (Id) is independently D, oxo, halogen, —CN, —NH₂, —NH(CH₂), —N(CH₃)₂, —OH, —CO₂H, —CO₂(C₁-C₄alkyl), —C(═O)NH₂, —C(═O)NH(C₁-C₄ alkyl), —C(═O)N(C₁-C₆ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₃ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, or —S(═O)—(C₁-C₄ alkyl). In some embodiments, each R^{Q} of Formula (Ia), Formula (Ib) or Formula (Id) is independently D, oxo, halogen, —CN, —NH₂, —OH, —NH(CH₃), —N(CH₃)₂, —NH(cyclopropyl), —CH₃, —CH₂CH₃, —CF₃, —OCH₃, or —OCF₃. In some embodiments, each R^{Q} is independently substituted or unsubstituted C₁-C₃ alkyl, amino, or —CN. In some embodiments, each R^{Q} is independently methyl, —CH₂CN, or CN.

In some embodiments, the structure of Formula (Id) is

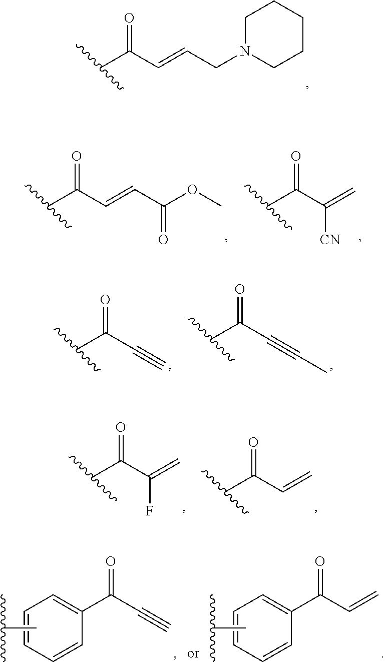

In some embodiments of Formula (Ib), R¹ is hydrogen, substituted or unsubstituted C₁-C₆ alkyl, substituted or unsubstituted C₁-C₆ heteroalkyl, substituted or unsubstituted C₁-C₆ cycloalkyl, or substituted or unsubstituted C₂-C₅ heterocycloalkyl. In some embodiments, R¹ is hydrogen or substituted or unsubstituted C₁-C₆ alkyl. In some embodiments, R¹ is hydrogen. In some embodiments, R¹ is substituted or unsubstituted C₃-C₆ cycloalkyl. In some embodiments, R¹ is substituted or unsubstituted C₂-C₅ heterocycloalkyl. In some embodiments, R¹ is substituted or unsubstituted C₁-C₆, heteroalkyl.

In some embodiments of Formula (Ia), Formula (Ib) or Formula (Id), R² is hydrogen or substituted or unsubstituted C₁-C₃ alkyl. In some embodiments, R² is hydrogen. In some embodiments of Formula (Ia) or Formula (Ib), R² is a substituted C₁-C₃ alkyl. In some embodiments of Formula (Ia) or Formula (Ib). R² is an unsubstituted C₁-C₃ alkyl. In some embodiments, R² is methyl.

In some embodiments of Formula (Ia), Formula (Ib) or Formula (Id), R⁵ and R⁷ are each independently selected from hydrogen, —CN, halogen, substituted or unsubstituted C₁-C₄ alkyl, substituted or unsubstituted C₁-C₄ heteroalkyl, substituted or unsubstituted C₃-C₆ cycloalkyl, or substituted or unsubstituted C₂-C₅ heterocycloalkyl. In some embodiments of Formula (Ia), Formula (Ib) or Formula (Ib). R⁵ and R⁷ taken together form a bond.

In some embodiments of Formula (Ia), Formula (Ib) or Formula (Id). R⁵ is a halogen. In some embodiments of Formula (Ia), Formula (Ib) or Formula (Id), R⁵ is hydrogen. In some embodiments of Formula (Ia), Formula (Ib) or Formula (Id), R⁵ is fluorine or chlorine. In some embodiments of Formula (Ia), Formula (Ib) or Formula (Id). R⁵ is —CN. In some embodiments of Formula (Ia), Formula (Ib) or Formula (Id). R⁵ is methyl or —O-Me. In some embodiments, R⁵ is hydrogen, halogen, methyl, or —OMe.

In some embodiments of Formula (Ia), Formula (Ib) or Formula (Id). R⁷ is substituted or unsubstituted C₂-C₅ heterocycloalkyl. In some embodiments of Formula (Ia), Formula (Ib) or Formula (Id), R⁷ is substituted or unsubstituted C₁-C₄ heteroalkyl. In some embodiments of Formula (Ia), Formula (Ib) or Formula (Id). R⁷ is hydrogen. In some embodiments, R⁷ is substituted or unsubstituted C₁-C₄ alkyl. In some embodiments, R⁷ is —CH₂F, —CH₂OMe, or —CH₂ C₂-C₅ heterocycloalkyl.

In some embodiments of Formula (Ia), Formula (Ib) or Formula (Id). R⁶ is hydrogen, halogen, C₁-C₃ alkyl, C₁-C₃ heteroalkyl, C₁₋₃alkylaminyl-C₁₋₃alkyl, di(C₁₋₃)alkylaminyl-C₁₋₃alkyl, C₃-C₆cycloalkyl or C₂-C₅ heterocycloalkyl, each of which is optionally substituted. In some embodiments of Formula (Ia), Formula (Ib) or Formula (Id), R⁶ is an unsubstituted or substituted C₁-C₃heteroalkyl, alkylaminylalkyl, or dialkylaminylalkyl. In some embodiments of Formula (Ia), Formula (Ib) or Formula (Id). R⁶ is an unsubstituted or substituted heterocycloalkyl. In some embodiments of Formula (Ia), Formula (Ib) or Formula (Id), R⁶ is hydrogen. In some embodiments of Formula (Ia), Formula (Ib) or Formula (Id), R⁶ is an unsubstituted or substituted heteroaryl. In some embodiments, R⁶ is a substituted 5 or 6-membered heteroaryl. In some embodiments of Formula (Ia), Formula (Ib) or Formula (Id). R⁶ is an unsubstituted or substituted aryl. In some embodiments, R⁶ is a monocyclic ring. In some embodiments, R⁶ is a bicyclic ring.

In some embodiments of Formula (Ia), Formula (Ib), Formula (Ic) or Formula (Id), each of R⁵, R⁶ and R⁷ is hydrogen. In some embodiments of Formula (Ia), Formula (Ib), Formula (Ic) or Formula (Id), R⁵ is fluorine and, R⁶ and R⁷ is hydrogen. In some embodiments of Formula (Ia), Formula (Ib), Formula (Ic) or Formula (Id), R⁵ is —CH₃ and, R⁶ and R⁷ is hydrogen. In some embodiments of Formula (Ia), Formula (Ib), Formula (Ic) or Formula (Id), R⁵ is —OCH₃ and, R⁶ and R⁷ is hydrogen. In some embodiments of Formula (Ia), Formula (Ib), Formula (Ic) or Formula (Id). R⁵ and R⁶ are hydrogen and R⁷ is —CH₂F. In some embodiments of Formula (Ia), Formula (Ib), Formula (Ic) or Formula (Id). R⁵ and R⁶ are hydrogen and R⁷ is —CH₂OMe. In some embodiments of Formula (Ia), Formula (Ib), Formula (Ic) or Formula (Id). R⁵ and R⁶ are hydrogen and R⁷ is —CH₂C₂-C₅heterocycloalkyl. In some embodiments of Formula (Ia), Formula (Ib), Formula (Ic) or Formula (Id), R⁵ and R⁶ are hydrogen and R⁷ is —CH₂aziridinyl. In some embodiments of Formula (Ia), Formula (Ib), Formula (Ic) or Formula (Id), R⁵ and R⁶ are hydrogen and R⁷ is —CH₂-azetidinyl. In some embodiments of Formula (Ia), Formula (Ib), Formula (Ic) or Formula (Id), R⁵ and R⁶ are hydrogen and R⁷ is —CH₂-pyrrolidinyl. In some embodiments of Formula (Ia), Formula (Ib), Formula (Ic) or Formula (Id), R⁵ and R⁶ are hydrogen and R⁷ is —CH₂-piperidinyl.

In some embodiments, an electrophilic functional group described herein comprises a structure selected from

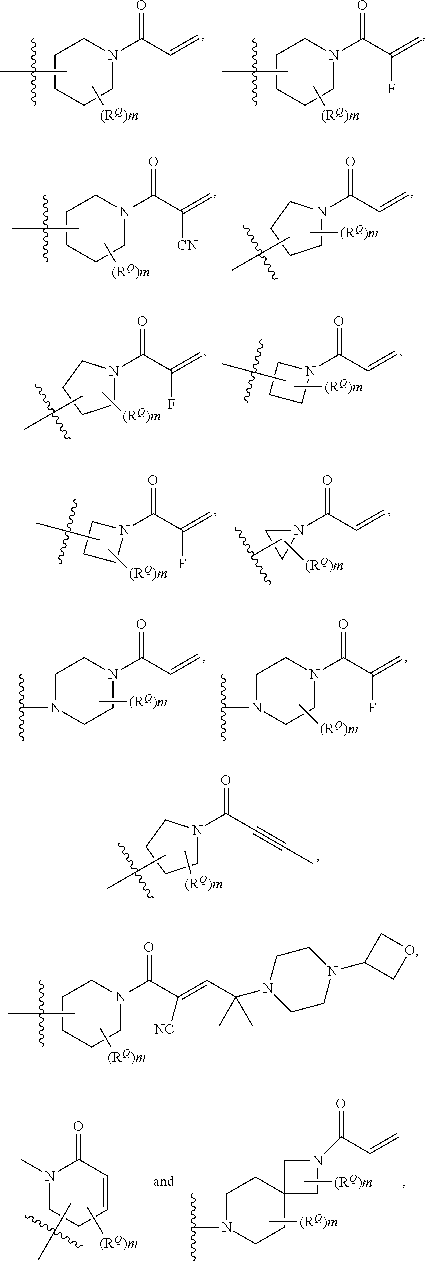

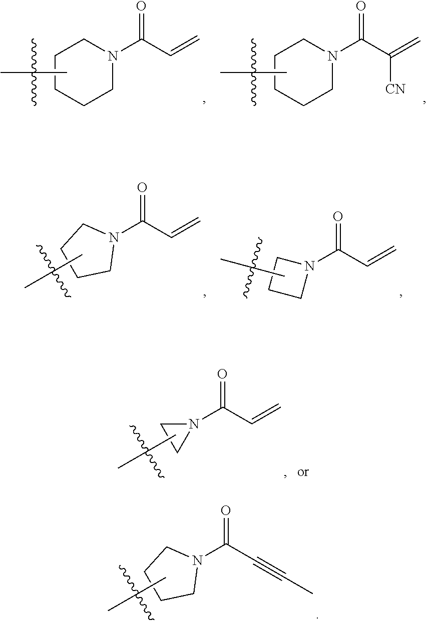

In some embodiments, the electrophilic functional group comprises

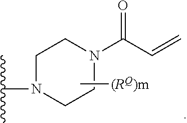

In some embodiments, the electrophilic functional group comprises

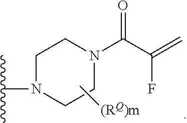

In some embodiments, the electrophilic functional group comprises (R)ₙ in some embodiments, R^{Q} is independently D, oxo, halogen, —CN, —NH₂, —OH, —NH(C₁-C₃alkyl), —N(C₁-C₃alkyl)₂, —NH(cyclopropyl), C₁-C₆ alky, or C₁-C₆ alkoxyl, wherein the alkyl or alkoxyl is optionally substituted with —CN and/or one or more halogens. In some embodiments, R^{Q} is independently substituted or unsubstituted C₁-C₃ alkyl, amino, or —CN, where the alkyl is optionally substituted with —CN and/or one or more halogens. In some embodiments, R^{Q} is C₁-C₃ alkyl substituted with —CN. In some embodiments, R^{Q} is C₁-C₃ alkyl substituted with one, two, or three fluorine atoms.

In some embodiments, an electrophilic functional group described herein comprises a structure selected from

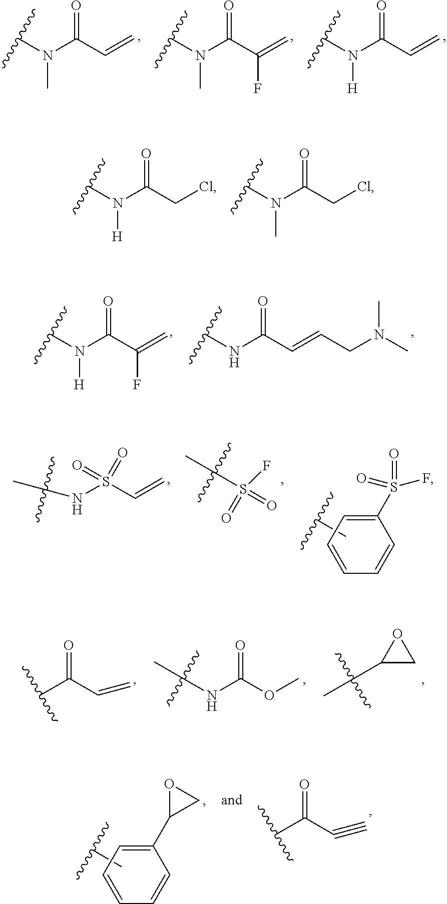

wherein the phenyl rings are optionally substituted. In some embodiments, the electrophilic functional group comprises

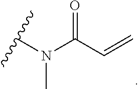 [SMILES: *N(C)C(=O)C=C]

In some embodiments, the electrophilic functional group comprises

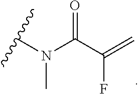 [SMILES: *N(C)C(=O)C(=C)F]

In some embodiments, the electrophilic functional group comprises

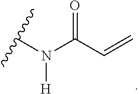 [SMILES: *NC(=O)C=C]

In some embodiments, the electrophilic functional group comprises

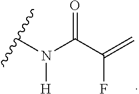 [SMILES: *NC(=O)C(=C)F]

In some embodiments, R⁵ and R⁷ taken together form a bond. In some embodiments, E comprises a structure of Formula (Ic), wherein the structure of Formula (Ic) is

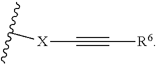

In some embodiments, the structure of Formula (Id) is

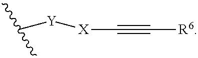

In some embodiments, an electrophilic functional group described herein comprises

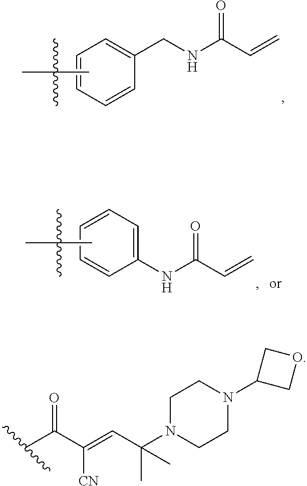

In some embodiments, an electrophilic functional group described herein comprises the structure

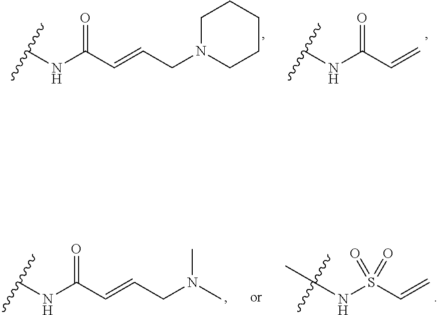

In some embodiments, an electrophilic functional group described herein comprises

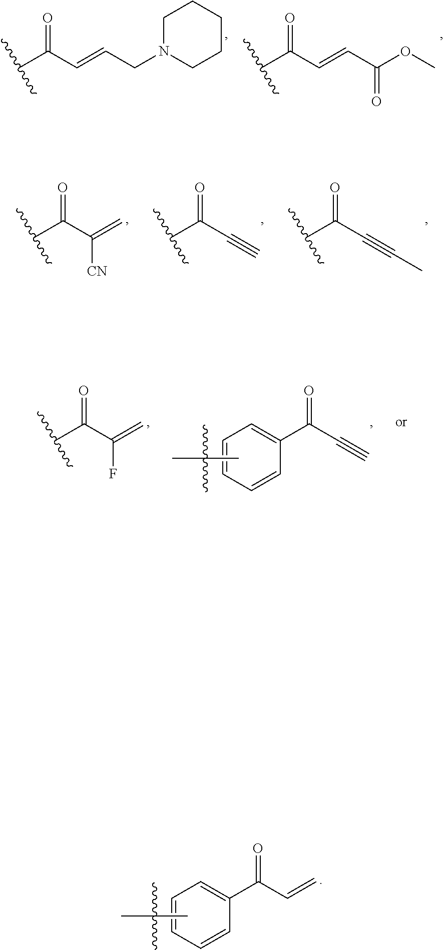

In some embodiments, an electrophilic functional group of the radiolabeled compound covalently binds to the KRAS G12C mutated protein at the cysteine residue 12. The radiolabeled compound can bind to a mutant-specific cysteine residue of the mutated KRAS G12C protein. The radiolabeled compound can form a covalent bond with a mutant-specific cysteine residue of the mutated KRAS protein. The mutant-specific cysteine residue of the mutated KRAS protein can be G12C.

Exemplary configurations of the radiolabeled compound described herein are illustrated in Table 4A-4D and Table 5A-5D.

In some embodiments, a covalently modified KRAS protein as described herein comprises 1) a covalently bonded radioisotope and 2) a radiolabeled compound comprising a structure of Formula (III), Formula (IIIa), Formula (IIIb), Formula (IIIc), Formula (IV), Formula (IVa), Formula (IVb), Formula (IVc), Formula (IVd), or Formula (IVe) or a salt, solvate, or derivative thereof as described herein.

In some embodiments, a covalently modified KRAS protein as described herein comprises a linker connecting the radioisotope and the electrophilic functional groups. The linker can comprise one or more structures of Tables 3A, 3B and 3C.

In some embodiments, a covalently modified KRAS protein described herein comprises a radiolabeled compound of Table 4A, or a salt or solvate thereof. In some embodiments, a covalently modified KRAS protein described herein comprises a radiolabeled compound of Table 4B, or a salt or solvate thereof. In some embodiments a covalently modified KRAS protein described herein comprises a radiolabeled compound of Table 4C, or a salt or solvate thereof. In some embodiments a covalently modified KRAS protein described herein comprises a radiolabeled compound of Table 4D, or a salt or solvate thereof. In some embodiments, the radiolabeled compound comprises a radioisotope such as bound to the linker.

In some embodiments, a covalently modified KRAS protein described herein comprises a radiolabeled compound of Table 5A, or a salt or solvate thereof. In some embodiments, a covalently modified KRAS protein described herein comprises a radiolabeled compound of Table 5B, or a salt or solvate thereof. In some embodiments a covalently modified KRAS protein described herein comprises a radiolabeled compound of Table 5C, or a salt or solvate thereof. In some embodiments a covalently modified KRAS protein described herein comprises a radiolabeled compound of Table 5D, or a salt or solvate thereof. In some embodiments, the radiolabeled compound comprises a radioisotope such as ²²⁵Ac or ¹⁷⁷Lu.

### III. Radiopharmaceutical Conjugates

Provided herein are radiolabeled compounds and pharmaceutical compositions comprising the radiolabeled compounds. The radiolabeled compounds and compositions can be useful for treating cancer. The compounds and compositions can also be useful in imaging and disease diagnosis.

In one aspect, described herein is a radiolabeled compound that binds to an intracellular mutated KRAS protein, optionally comprising a linker, and a radioisotope covalently bonded to the radiolabeled compound or the linker. In some embodiments, the radiolabeled compound can form an irreversible covalent bond to a KRAS protein. In some embodiments, the KRAS protein is mutated. In another aspect, described herein is a radiopharmaceutical conjugate comprising a) a targeting ligand that covalently binds to an intracellular KRAS protein, wherein the intracellular KRAS protein is mutated, and b) a radionuclide.

In some embodiments, the KRAS mutation comprises a glycine to cysteine mutation at amino acid residue 12 (G12C mutation). In some embodiments, the radiolabeled compound descried herein forms a bond with the KRAS protein at G12C position. In some embodiments, the radiolabeled compound comprises a radioisotope such as ¹³¹I bound to the compound. In some embodiments, the radiolabeled compound comprises the linker and the radioisotope such as ¹³¹I is bound to the linker.

In some embodiments, described herein is a radiolabeled compound comprising: (a) a moiety that covalently binds a mutated KRAS protein at G12C position (e.g., a targeting ligand). (b) a linker that covalently attaches the moiety to a radioisotope, and (c) a covalently bound radioisotope. The radiolabeled compound can form a covalent bond with the mutated KRAS protein at G12C position. In some embodiments, the radiolabeled compound comprises a radionuclide such as ¹³¹I bound to the linker.

In one aspect, provided herein is a radiolabeled compound that comprises an electrophilic functional group of Formula (Ia), Formula (Ib), Formula (Ic), or Formula (Id).

In one aspect, provided herein is a radiolabeled compound of Formula (III), or a salt or solvate thereof
wherein

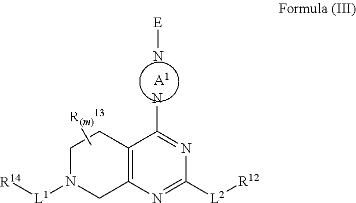

ring Q¹ is a 4-12 membered saturated or partially saturated monocyclic or bicyclic ring, wherein the monocyclic or bicyclic ring is optionally substituted;
   L¹ is a bond, —C(═O)—, O, S, NR'S, optionally substituted C₁-C₃ alkylene, optionally substituted C₁-C₃ heteroalkylene;
   L² is a bond, —C(═O)—, O, S or NR¹⁵;
E is

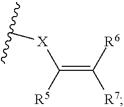

X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆ alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆ alklene-heteroraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R¹² is hydrogen, C₁-C₆ alkyl, C₂-C₆ heteroalkyl, -L³-NR¹⁵R^{15′}, heterocycloalkyl, -L³-heterocycloalkyl, cycloalkyl, -L³ cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
L³ is C₁-C₃ alkylene or C₁-C₄ heteroalkylene, each of which is optionally substituted;
each R¹³ is independently OH, halogen, oxo, substituted or unsubstituted C₁-C₆ alkyl, or substituted or unsubstituted C₁-C₆ heteroalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or optionally substituted C₁-C₃ alkyl;
each R^{15′} is independently hydrogen, acyl, optionally substituted C₁-C₃ alkyl, optionally substituted C₁-C₃ heteroalkyl or optionally substituted C₁-C₃ hydroxyalkyl; and
m is 0, 1, or 2;
provided that at least one of L¹, L², R¹², R¹³ and R¹⁴ comprises a covalently bonded radioisotope R.

In some embodiments, R* is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, R* is ¹³¹I. In some embodiments, R* is selected from a radioisotope in Table 6C or Table 6D.

In one aspect, provided herein is a radiolabeled compound of Formula (III), or a salt or solvate thereof
wherein

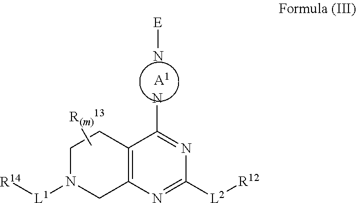

ring Q¹ is a 4-12 membered saturated or partially saturated monocyclic or bicyclic ring, wherein the monocyclic or bicyclic ring is optionally substituted;
   L¹ is a bond, —C(═O)—, or optionally substituted C₁-C₃ alkylene;
   L² is a bond, —C(═O)—, O, S or NR¹⁵;
E is

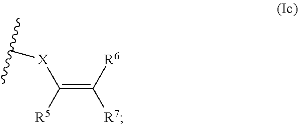

X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₆ heterocycloalkyl, wherein each of the alkyl, alkoxyl, heteroalkyl, cycloalkyl or heterocycloalkyl is optionally substituted, and
R⁷ is hydrogen, cyan, halogen, C₁-C₆ alkyl, C₁-C₆ alkoxyl, C₁-C₆ heteroalkyl, C₃-C₇ cycloalkyl, C₂-C₇ heterocycloalkyl, or heteroaryl, wherein each of the alkyl, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R₆ is hydrogen, cyano, halogen, C₁-C₆ alkyl, C₁-C₆ alkoxyl, C₁-C₆ heteroalkyl, C₁-C₆ cycloalkyl, C₂-C₆ heterocycloalkyl, or heteroaryl, wherein each of the alkyl, alkoxyl, heteroalkyl, cycloalkyl, heterocycloalkyl, or heteroaryl is optionally substituted;
R¹² is hydrogen, C₁-C₆ alkyl, C₁-C₆heteroalkyl, -L³-NR¹⁵R^{15′}, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted;
each R¹³ is independently OH, halogen, oxo, substituted or unsubstituted C₁-C₆ alkyl, or substituted or unsubstituted C₁-C₆ heteroalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or C₁-C₃ alkyl;
each R^{15′} is independently hydrogen, acyl, C₁-C₃ alkyl, C₁-C₃ heteroalkyl or C₁-C₃ hydroxyalkyl;
m is 0, 1, or 2.
wherein the structure of Formula (III) is attached to the rest of the conjugate at any suitable position; and
(b) a radionuclide.

In some embodiments,
ring Q¹ is a 4-12 membered saturated or partially saturated monocyclic or bicyclic ring optionally substituted with one, two, or three groups selected from R¹⁸;
L¹ is a bond, —C(═O)—, or optionally substituted C₁-C₃ alkylene, wherein the alkylene is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, and C₂-C₆ heterocycloalkyl;
L² is a bond, —C(═O)—, O, S or NR¹⁵;
E is

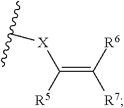

X is C(═O), P(═O)OR², C(═S), or S(═O)ₙ, where n is 1 or 2;
R² is hydrogen or substituted or unsubstituted C₁-C₆ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₅ heterocycloalkyl;
R⁵ is hydrogen, cyano, halogen, C₁-C₆ alkyl, C₁-C₆ alkoxyl, C₁-C₆ heteroalkyl, C₁-C₆ cycloalkyl, or C₂-C₆ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅heterocycloalkyl;
R⁷ is hydrogen, cyano, halogen, C₁-C₆ alkyl, C₁-C₆ alkoxyl, C₁-C₆ heteroalkyl, C₃-C₆ cycloalkyl, C₂-C₇ heterocycloalkyl, or C₁-C₉heteroaryl, wherein each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, and C₂-C₅ heterocycloalkyl, or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyano, halogen, C₁-C₆ alkyl, C₁-C₆ alkoxyl, C₁-C₆ heteroalkyl, C₃-C₆ cycloalkyl, C₂-C₆ heterocycloalkyl, or C₁-C₉ heteroaryl, wherein, each of the alkyl, alkoxyl, heteroalkyl, cycloalkyl, heterocycloalkyl, or heteroaryl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅ heterocycloalkyl;
R¹² is hydrogen, C₁-C₆ alkyl, C₁-C₆ heteroalkyl, -L³-NR¹⁵R^{15′}, C₂-C₁₂heterocycloalkyl, -L³-C₂-C₁₂heterocycloalkyl, C₃-C₁₅cycloalkyl, -L³-C₃-C₁₅cycloalkyl, aryl, C₁-C₉heteroaryl, -L³-aryl, or -L³-C₁-C₉ heteroaryl, wherein each of the heterocycloalkyl, cycloalkyl, aryl, heteroaryl, alkyl or heteroalkyl is optionally substituted with one, two, three or four groups selected from R¹⁹;
L³ is C₁-C₄ alkylene or C₁-C₄ heteroalkylene, each of which is optionally substituted with one, two, or three groups selected from R¹⁹;
each R¹³ is independently OH, halogen, oxo, substituted or unsubstituted C₁-C₆ alkyl, or substituted or unsubstituted C₁-C₆ heteroalkyl, wherein each of the alkyl and heteroalkyl is optionally substituted with one, two, or three groups selected from halogen, —CN, hydroxy, C₁-C₆ alkyl, C₁-C₆ haloalkyl, C₁-C₆ alkoxy-, C₁-C₆cycloalkyl, and C₂-C₅heterocycloalkyl;
R¹⁴ is hydrogen, C₃-C₁₅cycloalkyl, C₂-C₁₂heterocycloalkyl, aryl, or C₁-C₉ heteroaryl, wherein each of the cycloalkyl, heterocycloalkyl, aryl, and heteroaryl is optionally substituted with one, two, three or four groups selected from R¹⁶;
each R¹⁵ is independently hydrogen or C₁-C₃ alkyl;
each R^{15′} is independently hydrogen, acyl, C₁-C₃ alkyl, C₁-C₃ heteroalkyl or C₁-C₃ hydroxyalkyl;
m is 0, 1, or 2;
each R¹⁷ is independently halogen, hydroxyl, C₁-C₆ alkyl, C₂-C₅cycloalkyl, C₁-C₆ alkoxy, C₁-C₆ haloalkyl, amino, cyano, C₁-C₆ heteroalkyl, C₁-C₆ hydroxyalkyl, —O—C₁-C₆haloalkyl, or —S—C₁-C₆haloalkyl;
each R¹⁸ is independently halogen, oxo, C₁-C₆ alkyl, C₁-C₆ aminoalkyl, C₁-C₆ haloalkyl, C₂-C₇ alkenyl, C₂-C₅ alkynyl, C₁-C₆ heteroalkyl, —CN, —C(O)OR¹⁵, —C(O)N(R¹⁵)(R^{15′}), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₂-C₅ haloalkyl, C₁-C₆ alkoxy, C₃-C₆ cycloalkyl, and C₂-C₆ heterocycloalkyl;
R¹⁶ and R¹⁹ are each independently selected from halogen, oxo, —CN, C₁-C₆ alkyl, C₂-C₆ alkenyl, C₂-C₆ alkynyl, C₁-C₆ heteroalkyl, C₃-C₁₀ cycloalkyl, —C₁₋₃alkylene-C₃₋₆cycloalkyl, C₂-C₉ heterocycloalkyl, —C₁₋₃alkylene-C₂₋₉heterocycloalkyl, C₆-C₁₀ aryl, —C₁₋₃alkylene-C₆₋₁₀aryl, C₁-C₉ heteroaryl, —C₁₋₃alkylene-C₁₋₉ heteroaryl, —OR¹⁰, —SR¹⁰, —N(R¹⁰)(R^{10′}), —C(O)OR¹⁰, OC(═O)N(R¹⁰)(R^{10′}), —N(R^{10′})C(═O)N(R¹⁰)(R^{10′}), —N(R^{10′})C(O)OR¹¹, —N(R^{10′})S(═O)₂R¹¹, —C(═O)R¹¹, —S(C)R¹¹, —OC(═O)R¹¹, —C(═)N(R¹⁰)(R^{10′}), —C(═)C(═O)N(R¹⁰)(R^{10′}), —N(R^{10′})C(O)R¹¹, —S(O)₂R¹¹, —S(O)₂N(R¹⁰)(R^{10′}), —S(═O)(═NH)N(R¹⁰)(R^{10′}), —CH₂C(O)N(R¹⁰)(R^{10′}), —CH₂N(R^{10′})C(═O)R¹¹, —CH₂S(═O)₂R¹¹, and —CH₂S(═O)₂N(R¹⁰)(R^{10′}), wherein the alkyl, alkenyl, alkynyl, cycloalkyl, —C₁₋₃alkylene-C₃₋₆cycloalkyl, heterocycloalkyl, —C₁₋₃alkylene-C₂-C₉heterocycloalkyl, aryl, —C₁₋₃alkylene-C₆₋₁₀aryl, heteroaryl and —C₁₋₃alkylene-C₁₋₉ heteroaryl are optionally substituted with one, two, three, or four groups independently selected from halogen, oxo, —CN, C₁-C₆ alkyl, C₁-C₆ haloalkyl, C₁-C₆ alkoxy, C₁-C₆ alkyloxy, C₁-C₆ haloalkoxy, C₃-C₁₀ cycloalkyl, C₂-C₉ heterocycloalkyl, C₆-C₁₀ aryl, C₁-C₉ heteroaryl, —OR¹⁰, —SR¹⁰, —N(R¹⁰)(R^{10′}), —C(═O)OR¹⁰, —OC(═O)N(R¹⁰)(R^{10′}), —N(R^{10′})C(═O)N(R¹⁰)(R^{10′}), —N(R^{10′})C(═O)OR¹¹, —N(R^{10′})S(═O)₂R^{10′}, —C(═O)R¹¹, —S(O)R¹¹, —OC(═O)R¹¹, —C(═O)N(R¹⁰)(R^{10′}), —C(═O)C(═O)N(R¹⁰)(R^{10′}), —N(R¹⁰)C(═O)R¹¹, —S(═O)₂R¹¹, —S(═O)₂N(R¹⁰)(R^{10′})—, S(═O)(═NH)N(R¹⁰)(R^{10′}), —CH₂C(O)N(R¹⁰)(R^{10′}), —CH₂N(R^{10′})C(═O)R¹¹. —CH₂S(═O)₂R¹¹, and —CH₂S(═O)₂N(R¹⁰)(R^{10′});
each R¹⁰ is independently selected from hydrogen, C₁-C₆ alkyl, C₁-C₆ haloalkyl, C₂-C₆ alkenyl, C₂-C₆ alkynyl, C₃-C₆ cycloalkyl, C₂-C₉heterocycloalkyl, C₆-C₁₀ aryl, and C₁-C₉ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉heteroaryl;
each R^{10′} is independently selected from hydrogen, C₁-C₆ alkyl, and C₁-C₆ haloalkyl; and
each R¹¹ is independently selected C₁-C₆ alkyl, C₂-C₆ alkenyl, C₂-C₆ alkynyl, C₃-C₆cycloalkyl, C₂-C₉ heterocycloalkyl, C₆-C₁₀ aryl, and C₁-C₉ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉heteroaryl.

In some embodiments of Formula (III) or (X-III), Q¹ is optionally substituted with one or more R¹⁸, wherein R¹⁸ is oxo, C₁-C₆ alkyl, C₂-C₆ alkenyl, C₂-C₆ alkynyl, C₁-C₆, heteroalkyl, cyano, —C(O)OR¹⁵, —C(O)N(R¹⁵)(R^{15′}), —N(R¹⁵)(R^{15′}), wherein the alkyl, alkenyl, alkynyl, or heteroalkyl is optionally substituted. In some embodiments, Q¹ is optionally substituted with one to three R¹⁸, wherein R¹⁸ is independently halogen, oxo, C₁-C₆ alkyl, C₁-C₆ aminoalkyl, C₁-C₆ haloalkyl, C₂-C₆ alkenyl, C₂-C₆ alkynyl, C₁-C₆ heteroalkyl, —CN, —C(O)OR¹⁵, —C(O)N(R¹⁵)(R^{15′}), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₁-C₆ haloalkyl, C₁-C₆ alkoxy, C₁-C₆ cycloalkyl, and C₂-C₆ heterocycloalkyl. In some embodiments, Q¹ is a 6 membered monocyclic ring, where in the monocyclic ring is optionally substituted with one to three R¹⁸, wherein R¹⁸ is methyl, CN, —CH₂CN, carbonyl, hydroxyl, carboxyl, or C(O)OR¹⁵. In some embodiments, Q¹ is a 6 membered monocyclic ring substituted with —CH₂CN.

In some embodiments of Formula (III), Formula (IIIa), Formula (IIIb), Formula (IIIc), Formula (X-III), Formula (IV), Formula (IVa), Formula (IVb), Formula (IVc), Formula (IVd), and Formula (IVe), or Formula (X-IV), R⁵ is hydrogen, cyano, halogen, optionally substituted C₁-C₆ alkyl, optionally substituted C₁-C₆ alkoxyl, optionally substituted C₁-C₆ heteroalkyl, optionally substituted C₃-C₆ cycloalkyl, or optionally substituted C₂-C₅ heterocycloalkyl. In some embodiments, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, and C₂-C₅ heterocycloalkyl.

In some embodiments of Formula (III), Formula (IIIa), Formula (IIIb), Formula (IIIc), Formula (X-III), Formula (IV), Formula (IVa), Formula (IVb), Formula (IVc), Formula (IVd), and Formula (IVe), or Formula (X-IV), R⁵ is hydrogen or a C₁-C₃ alkyl optionally substituted by one to three substituents selected from hydroxyl and halogen. In some embodiments, R⁵ is a halogen. In some embodiments, R⁵ is C₁-C₆ heteroalkyl. In some embodiments, R⁵ is —C(O)NR¹⁵R^{15′}. In some embodiments, R⁵ is fluoro.

In some embodiments of Formula (III), Formula (IIIa), Formula (IIIb), Formula (IIIc), Formula (X-III), Formula (IV), Formula (IVa), Formula (IVb), Formula (IVc), Formula (IVd), and Formula (IVe), or Formula (X-IV), R⁷ is hydrogen, cyan, halogen, optionally substituted C₁-C₆ alkyl, optionally substituted C₁-C₆ alkoxyl, or optionally substituted C₁-C₆ heteroalkyl. In some embodiments, R⁷ is hydrogen. In some embodiments, R⁷ is C₁-C₆ heteroalkyl selected from —NHC(O)—C₁-C₃ alkyl and —CH₂NHC(O)—C₁-C₃ alkyl.

In some embodiments of Formula (III), Formula (IIIa), Formula (IIIb), Formula (IIIc), Formula (X-III), Formula (IV), Formula (IVa), Formula (IVb), Formula (IVc), Formula (IVd), and Formula (IVe), or Formula (X-IV), R⁵ and R⁷ taken together with the carbon atoms to which they are attached form a 5-8 membered partially saturated cycloalkyl, wherein the cycloalkyl is optionally substituted with one or more R¹⁷, wherein each R¹⁷ is independently halogen, hydroxyl, C₁-C₆ alkyl, cycloalkyl, alkoxy, haloalkyl, amino, cyano, heteroalkyl, hydroxyalkyl, —O-haloalkyl, or —S-haloalkyl.

In some embodiments of Formula (III), Formula (IIIa), Formula (IIIb), Formula (IIIc), Formula (X-III), Formula (IV), Formula (IVa), Formula (IVb), Formula (IVc), Formula (IVd), and Formula (IVe), or Formula (X-IV), R⁶ is hydrogen, cyano, halogen, optionally substituted C₁-C₆ alkyl, optionally substituted C₁-C₆ alkoxyl, optionally substituted C₁-C₆ heteroalkyl, optionally substituted C₃-C₆ cycloalkyl, or optionally substituted C₂-C₆ heterocycloalkyl. In some embodiments, R⁶ is hydrogen. In some embodiments. R⁶ is C₁-C₆ heteroalkyl selected from —NHC(O)—C₁-C₃alkyl and —CH₂NHC(O)—C₁-C₃alkyl.

In some embodiments of Formula (III), Formula (IIIa), Formula (IIIb), Formula (IIIc), or Formula (X-III), R¹⁴ is hydrogen, cycloalkyl, heterocycloalkyl, aryl, aralkyl, or heteroaryl, wherein each of the cycloalkyl, heterocycloalkyl, aryl, aralkyl, and heteroaryl is optionally substituted with one or more R¹⁶ wherein each R¹⁶ is independently halogen, hydroxyl, C₁-C₆ alkyl, cycloalkyl, alkoxy, acetyl, carboxyl, —C(O)OR¹⁵, haloalkyl, amino, cyano, heteroalkyl, hydroxyalkyl, —O-haloalkyl, or —S-haloalkyl. In some embodiments, R¹⁶ is halogen.

In some embodiments of Formula (III), Formula (IIIa), Formula (IIIb), Formula (IIIc), or Formula (X-III), R¹⁴ is aryl or heteroaryl, optionally substituted with one or more R¹⁶, wherein each R¹⁶ is independently D, halogen, —CN, —NH₂, —NH(alkyl), —N(alkyl)₂, —OH, oxo, —CO₂H, —CO₂alkyl, —C(═O)NH₂, —C(═O)NH(alkyl), —C(O)N(alkyl)₂, —S(═O)₂NH₂, —S(O)₂NH(alkyl), —S(O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

In some embodiments of Formula (III), Formula (IIIa), Formula (IIIb), Formula (IIIc), or Formula (X-III), R¹⁴ is aryl or heteroaryl, optionally substituted with one or more R¹⁶, wherein each R¹⁶ is independently D, amino, cyano, oxo, hydroxy, nitro, halogen, C₁₋₆ alkyl, C₁₋₆ alkenyl, C₁₋₆ alkoxy, C₃₋₇ cycloalkyl, aryl, heteroaryl, C₁₋₆heteroalkyl, C₂₋₇heterocycloalkyl, C₁₋₆alkyl-OH, trihalo-C₁₋₆ alkyl, mono-C₁₋₆ alkylamino, di-C₁₋₆ alkylamino, —C(═O)NH₂, hydroxy-C₁₋₆ alkylamino, hydroxy-C₁₋₆ alkyl, 4-7 membered heterocycle-C₁₋₆ alkyl, amino-C₁₋₆ alkyl, mono-C₁₋₆ alkylamino-C₁₋₆ alkyl, and di-C alkylamino-C₁ a alkyl.

In some embodiments of Formula (III), Formula (IIIa), Formula (IIIb), Formula (IIIc), or Formula (X-III), R¹⁴ is aryl or heteroaryl, optionally substituted with one or more R¹⁶, wherein each R¹⁶ is independently halogen, hydroxyl, C₁-C₃ alkyl, alkoxy, haloalkyl, amino, or cyano. In some embodiments, R¹⁴ is optionally substituted monocyclic heteroaryl. In some embodiments, R¹⁴ is optionally substituted bicyclic heteroaryl.

In some embodiments of Formula (III), Formula (IIIa), Formula (IIIb), Formula (IIIc), or Formula (X-III). R¹⁴ is an aryl. In some embodiments, R¹⁴ is phenyl. In some embodiments, R¹⁴ is napthyl. In some embodiments, R¹⁴ is an aryl substituted with one or more R¹⁶ groups. In some embodiments, R¹⁴ is napthyl substituted with one to three R¹⁶ groups. In some embodiments, R¹⁴ is napthyl substituted with one to three R¹⁶, wherein each R¹⁶ is independently halogen, hydroxyl, C₁-C₃ alkyl, alkoxy, haloalkyl, amino, or cyano.

In some embodiments of Formula (III), Formula (IIIa), Formula (IIIa-1), Formula (IIIa-2). Formula (IIIb), Formula (IIIc), or Formula (X-III), each R¹⁶ is independently D, oxo, halogen, —CN, —NH₂, —NH(CH₃), —N(CH₃)₂, —OH, —CO₂H, —CO₂(C₁-C₄alkyl), —OCO(C₁-C₁alkyl), —C(O)NH₂, —C(O)NH(C₁-C₄ alkyl), —C(═O)N(C₁-C₄alkyl)₂, —S(O)₂NH₂, —S(═O)₂NH(C₁-C₄alkyl), —S(═O)—N(C₁-C₄alkyl)₂, C₁-C₄alkyl, C₃-C₆cycloalkyl, C₁-C₄fluoroalkyl, C₁-C₆heteroalkyl, C₁-C₄ alkoxy, C₁-C₄fluoroalkoxy, —SC₁-C₄ alkyl. —S(═O)C₁-C₄ alkyl, or —S(═O)₂(C₁-C₄ alkyl). In some embodiments, each R¹⁶ is independently D, oxo, halogen, —CN, —NH₂, —OH, —NH(CH₃), —N(CH₃)₂, —NH(cyclopropyl), —CH₃. —CH₂CH₃, —CF₃, —OCH₃, or —OCF₃. In some embodiments, each R¹⁶ is independently substituted or unsubstituted C₁-C₃ alkyl, amino, or —CN.

In some embodiments of Formula (III), Formula (IIIa), Formula (IIIb), Formula (IIIc), or Formula (X-III), R¹⁴ is phenyl or naphthyl optionally substituted with one or more R¹⁶. In some embodiments, R¹⁴ is phenyl optionally substituted with one or more R¹⁶. In some embodiments, R¹⁴ is naphthyl optionally substituted with one or more R¹⁶. In some embodiments, each R¹⁶ is independently halogen, hydroxyl, C₁-C₃ alkyl, alkoxy, haloalkyl, amino, or cyano.

In some embodiments of Formula (III), Formula (IIIa), Formula (IIIb), Formula (IIIc), or Formula (X-III), R¹⁴ comprises the covalently bonded radioisotope R¹⁶. In some embodiments, R* is selected from a radioisotope in Table 6C or Table 6D. In some embodiments, R¹⁴ comprises a radionuclide selected from fluorine-18 (¹⁸F), iodine-131 (¹³¹I), iodine-123 (¹²³I), iodine-124 (¹²⁴I), iodine-125 (¹²⁴I), and astatine-211 (²¹¹At).

In some embodiments of Formula (III), Formula (IIIa), Formula (IIIa-1), Formula (IIIa-2), Formula (IIIb), Formula (IIIc), or Formula (X-III), R¹⁶ is halogen and the halogen is a radioisotope selected from iodine-131 (¹³¹I), iodine-123 (¹²³I), iodine-124 (¹²⁴I), and iodine-125 (¹²⁵I). In some embodiments, R¹⁶ is ¹³¹I. In some embodiments, R¹² is phenyl or naphthyl substituted with one or two R¹⁶ and each R¹⁶ is independently hydroxyl or ¹³¹I.

In some embodiments of Formula (III), Formula (IIIa), Formula (IIIb), Formula (IIIc), or Formula (X-III), R¹⁴ is

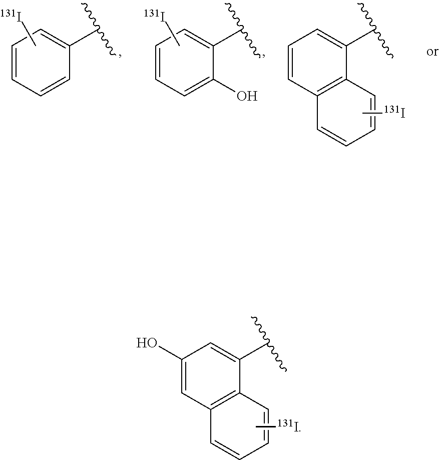

In some embodiments, R¹⁴ is

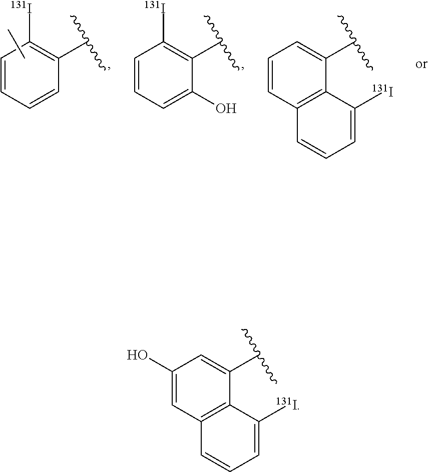

In some embodiments of Formula (III), or Formula (IIIa), R¹² is hydrogen, alkyl, heteroalkyl, -L³-alkylaminyl, -L³-dialkylaminyl. -L³-NR¹⁵R^{15′}, heterocycloalkyl, -L³-heterocycloalkyl, cycloalkyl, -L³-cycloalkyl, aryl, heteroaryl, -L³-aryl, or -L³-heteroaryl, wherein each of the L³, heterocycloalkyl, cycloalkyl, aryl, heteroaryl, alkyl or heteroalkyl is optionally substituted with one or more R¹⁹, wherein each R¹⁹ is independently hydrogen, oxo, acyl, hydroxyl, hydroxyalkyl, cyan, halogen, alkyl, aralkyl, heteroalkyl, cycloalkyl, heterocycloalkyl, alkoxy, dialkylaminyl, dialkylamidoalkyl, or dialkylaminylalkyl, wherein the alkyl, aralkyl, heteroalkyl, cycloalkyl, or heterocycloalkyl is optionally substituted.

In some embodiments of Formula (III), Formula (IIIa), Formula (IIIb), Formula (IIIc), or Formula (X-III), R¹² is alkyl, heteroalkyl, -L³-alkylaminyl, -L³dialkylaminyl, -L³-NR¹⁵R^{15′}, heterocycloalkyl, -L³-heterocycloalkyl, cycloalkyl, or -L³-cycloalkyl, wherein each of the L³, heterocycloalkyl, cycloalkyl, alkyl, or heteroalkyl is optionally substituted with one or more R¹⁹. In some embodiments, R¹² is optionally substituted C₁-C₆ alkyl. In some embodiments, R¹² is optionally substituted C₁-C₆ heteroalkyl.

In some embodiments of Formula (III), Formula (IIIa), Formula (IIIb), Formula (IIIc), or Formula (X-III). R¹² is optionally substituted C₁-C₆ alkyl. In some embodiments, R¹² is optionally substituted C₁-C₆ heteroalkyl. In some embodiments, R¹² is optionally substituted monocyclic heterocycloalkyl. In some embodiments. R¹² is optionally substituted pyrrolidine. In some embodiments, R¹² is optionally substituted bicyclic heterocycloalkyl. In some embodiments, R¹² is optionally substituted monocyclic cycloalkyl. In some embodiments, R¹² is optionally substituted bicyclic cycloalkyl.

In some embodiments of Formula (III), Formula (IIIa), Formula (IIIb), Formula (IIIc), or Formula (X-III), R¹² is -L³-C₁₋₆alkylaminyl, -L³-C₁₋₆dialkylaminyl, each of which is optionally substituted.

In some embodiments of Formula (III), Formula (IIIa), Formula (IIIb), Formula (IIIc), or Formula (X-III), R¹² is -L³-heterocycloalkyl. In some embodiments, R¹² is -L³-heterocycloalkyl, wherein the heterocycloalkyl is an optionally substituted 4-6 membered ring with 1-3 nitrogen atoms. In some embodiments, R¹² is -L-heterocycloalkyl, wherein the heterocycloalkyl is an optionally substituted monocyclic heterocycloalkyl. In some embodiments, R¹² is -L³-heterocycloalkyl, wherein the heterocycloalkyl is an optionally substituted bicyclic heterocycloalkyl. In some embodiments, R¹² is -L³-heterocycloalkyl, wherein the heterocycloalkyl is an optionally substituted 5-membered ring with 1 nitrogen atom.

In some embodiments of Formula (III), Formula (IIIa), Formula (IIIb), Formula (IIIc), or Formula (X-III), R¹² is C₃-C₁₅cycloalkyl, C₂-C₁₂heterocycloalkyl, -L³-C₂-C₁₂heterocycloalkyl, or -L³-C₃-C₁₅cycloalkyl, wherein each of the L³, heterocycloalkyl, cycloalkyl, alkyl, or heteroalkyl is optionally substituted with one, two, three or four groups selected from R¹⁹; and wherein each R¹⁹ is independently selected from oxo, —CN, C₁-C₆ alkyl, C₂-C₅ heteroalkyl, C₃-C₁₀cycloalkyl, —C₁₋₃alkylene-C₁₋₆cycloalkyl, C₂-C₉ heterocycloalkyl, —C₁₋₃alkylene-C₂₋₄heterocycloalkyl, C₆-C₁₀ aryl, —C₁₋₃alkylene-C₁-C₆aryl, C₁-C₉ heteroaryl, —C₁₋₃alkylene-C₁, heteroaryl, —OR¹⁰, —SR, —N(R¹⁰)(R^{10′}), —C(O)OR¹⁰, —OC(═O)N(R¹⁰)(R^{10′}), —N(R¹⁰)C(═O)N(R¹⁰)(R^{10′}), —N(R^{10′})(═O)OR^{a}, —N(R^{10′})S(═O)₂R¹¹, —C(═O)R¹¹, —S(C)R¹¹, —OC(═O)R¹¹, —C(═O)N(R¹⁰)(R^{10′}), —C(═O)C(═O)N(R¹⁰)(R^{10′}), —N(R^{10′})C(O)R¹¹, —S(O)₂R¹¹, —S(O)₂N(R¹⁰)(R^{10′}), —S(═O)(═NH)N(R¹⁰)(R^{10′}), —CH—C(O)N(R¹⁰)(R^{10′}), —CH₂N(R¹⁰)C(═O)R¹¹, —CH₂S(═O)—R¹¹, and —CH₂S(═O)₂N(R¹⁰)(R^{10′}), wherein the alkyl, alkenyl, alkynyl, cycloalkyl, —C₁-C₃alkylene-C cycloalkyl, heterocycloalkyl, —C₁₋₃alkylene-C₂-C₉heterocycloalkyl, aryl, —C₁₋₃alkylene-C₆₋₁₀aryl, heteroaryl and —C₁₋₃alkylene-C₁₋₉ heteroaryl are optionally substituted with one, two, three, or four groups independently selected from halogen, oxo, —CN, C₁-C₆ alkyl, C₁-C₆ haloalkyl, C₁-C₆ alkoxy, C₁-C₆ alkyloxy, C₁-C₆ haloalkoxy, C₃-C₁₀ cycloalkyl, C₂-C₉ heterocycloalkyl, C₆-C₁₀ aryl, C₁-C₉ heteroaryl, —OR¹⁰, —SR¹⁰. —N(R¹⁰)(R^{10′}), —C(═O)OR¹⁰, —OC(═O)N(R¹⁰)(R^{10′}), —N(R^{10′})C(═O)N(R¹⁰)(R^{10′}), —N(R^{10′})C(═O)OR^{a}, —N(R¹¹)S(═O)—R¹¹, —C(═O)R¹¹, —S(O)R¹¹, —OC(═O)R¹¹, —C(═O)N(R¹⁰)(R^{10′}), —C(═O)C(═O)N(R¹⁰)(R^{10′}), —N(R¹⁰)C(═O)R¹⁰, —S(═O)₂R¹¹, —S(═O)₂N(R¹⁰)(R^{10′})—, S(O)(═NH)N(R¹⁰)(R^{10′}), —CH₂C(═O)N(R¹⁰)(R^{10′}), —CH₂N(R^{10′})C(═O)R¹¹, —CH₂S(═O)₂R¹¹, and —CH₂S(═O)₂N(R¹⁰)(R^{10′}).

In some embodiments of Formula (III), Formula (IIIa), Formula (IIIb), Formula (IIIc), or Formula (X-III), R¹² is -L³-C₂-C₁₂heterocycloalkyl, optionally substituted with one, two, three or four groups selected from R¹⁹; and wherein each R¹⁹ is independently selected from oxo, C₁-C₆ alkyl, C₁-C₆ heteroalkyl, —C₁₋₃alkylene-C₆₋₁₀aryl, —C₁₋₃alkylene-C₁₋₉heteroaryl, and OR¹⁰, wherein the alkyl, heteroalkyl, and —C₁₋₃alkylene-C₆₋₁₀aryl, and —C₁₋₃alkylene-C₁₋₉heteroaryl are optionally substituted with one, two, three, or four groups independently selected from C₁-C₆ alkyl, C₆-C₁₀ aryl, —OR, —C(═O)OR¹⁰, or —N(R¹⁰)C(═O)R¹¹. In some embodiments, R¹² is -L³-C₂-C₁₂heterocycloalkyl, optionally substituted with one, two, or three groups selected from R¹⁹ wherein L³ is methylene, the heterocycloalkyl is an optionally substituted 5-membered ring with 1 nitrogen atom, and each R¹⁹ is independently selected from oxo, C₁-C₆, alkyl, C₁-C₆ heteroalkyl, —C₁₋₃alkylene-C₆₋₁₀aryl, —C₁₋₃alkylene-C₁₋₉heteroaryl, and OR¹⁰, wherein the alkyl, heteroalkyl, and —C₁₋₃alkylene-C₆₋₁₀aryl, and —C₁₋₃alkylene-C₁₋₉heteroaryl are optionally substituted with one, two, three, or four groups independently selected from C₁-C₆ alkyl, C₆-C₁₀ aryl, —OR¹⁰, —C(═O)OR¹⁰, or —N(R¹⁰)C(═O)R¹¹.

In some embodiments, the structure of Formula (III), Formula (IIIa), Formula (IIIb), Formula (IIIc), or Formula (X-lii) is attached to the rest of the conjugate through R¹². In some embodiments, the structure of Formula (III), Formula (IIIa), Formula (IIIb), Formula (IIIc), or Formula (X-III) is attached to the linker or to the metal chelator via R¹² group. In some embodiments, the structure of Formula (III), Formula (IIIa), or Formula (III)) is attached to the rest of the conjugate through R¹⁴.

In some embodiments, the targeting ligand comprises a structure of Formula (IIIa-1) or Formula (IIIa-2), or a salt or solvate thereof,

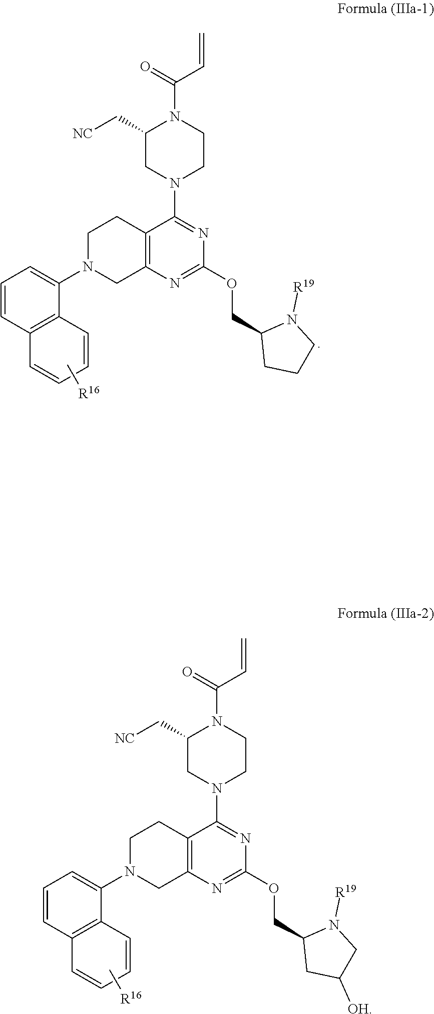

In some embodiments, the targeting ligand comprises a structure of Formula (IIIa-1) or a salt thereof. In some embodiments, the targeting ligand comprises a structure of Formula (IIIa-2) or a salt thereof.

In some embodiments, the structure of Formula (III), Formula (IIIa), Formula (IIIa-1), Formula (IIIa-2), Formula (IIIb), or Formula (IIIc) comprises the radionuclide through R¹⁹. In some embodiments of Formula (III), Formula (IIIa), Formula (IIIa-1), or Formula (IIIa-2). R¹⁶ is the radionuclide. In some embodiments of Formula (III), Formula (IIIa), Formula (IIIa-1), Formula (IIIa-2), Formula (IIIb), or Formula (IIIc), R¹⁹ is

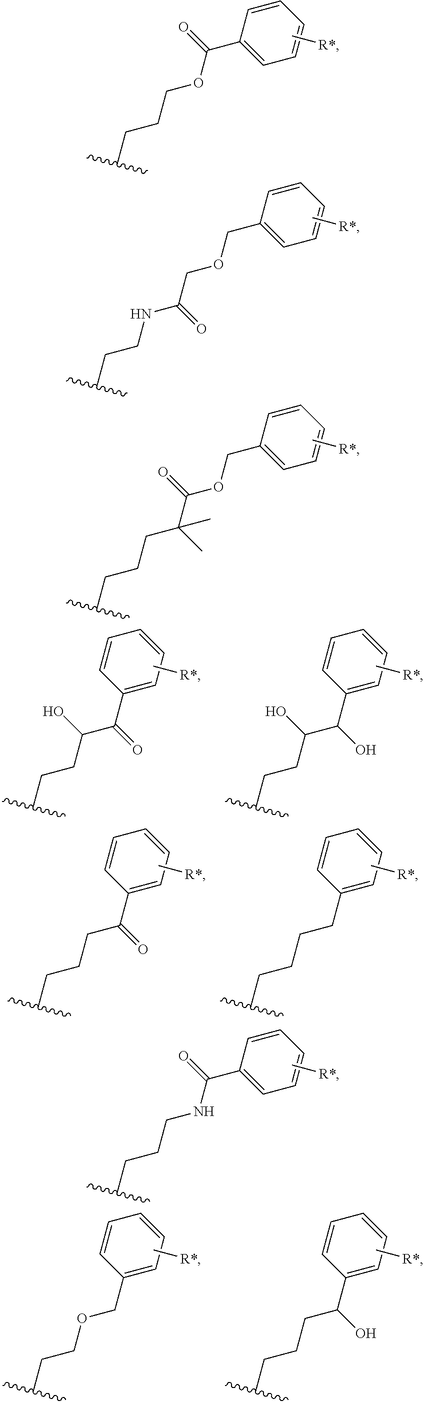

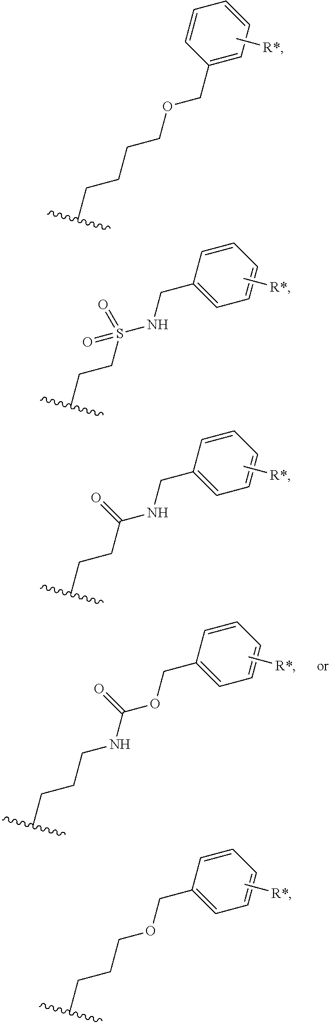

wherein R* is the radionuclide. In some embodiments, R* is selected from a radioisotope in Table 6C or Table 6D. In some embodiments, R* is iodine-131 (¹³¹I) or astatine-211 (²¹¹At).

In some embodiments of Formula (III), Formula (IIIa), Formula (IIIa-1), Formula (IIIa-2), Formula (IIIb), or Formula (IIIc), R¹⁹ is

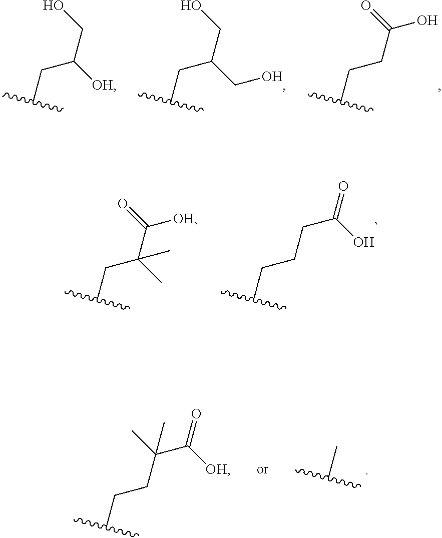

In some embodiments of Formula (III), Formula (IIIa), Formula (IM), Formula (IIIc), or Formula (X-III), R¹² is -L³-cycloalkyl. In some embodiments, R¹² is -L³-cycloalkyl, wherein the cycloalkyl is an optionally substituted 4-6 membered ring. In some embodiments, R¹² is -L³-cycloalkyl, wherein the cycloalkyl is an optionally substituted monocyclic ring. In some embodiments, R¹² is -L³-cycloalkyl, wherein the cycloalkyl is an optionally substituted bicyclic ring. In some embodiments, R¹² is 3 to 6 membered cycloalkyl.

In some embodiments of Formula (III), Formula (IIIa), Formula (IIIb), Formula (IIIc), or Formula (X-III). L³ is C₁-C₄ alkylene or C₁-C₄ heteroalkylene, each of which is optionally substituted with one or more R¹⁹. In some embodiments, L is an optionally substituted methylene. In some embodiments, L³ is an optionally substituted ethylene. In some embodiments, L³ is an optionally substituted C₂-C₄ alkylene. In some embodiments. L³ is an optionally substituted C₂-C₄ heteroalkylene.

In some embodiments of Formula (III), Formula (IIIa), Formula (IIIa-1), Formula (IIIa-2), Formula (IIIb), Formula (IIIc), or Formula (X-III), R¹⁹ is hydrogen, oxo, acyl, hydroxyl, hydroxyalkyl, cyano, halogen, alkyl, aralkyl, aryl heteroalkyl, cycloalkyl, heterocycloalkyl, alkoxy, dialkylaminyl, dialkylamidoalkyl, or dialkylaminylalkyl. In some embodiments, R¹⁹ is an optionally substituted alkyl, aralkyl, heteroalkyl, cycloalkyl, or heterocycloalkyl. In some embodiments, R¹⁹ is alkyl, aralkyl, heteroalkyl, cycloalkyl, or heterocycloalkyl substituted with one or more of hydrogen, hydroxyl, cyan, halogen, or C₁-C₃ alkyl. In some embodiments, R¹⁹ is an optionally substituted C₁-C₃ alkyl. In some embodiments, R¹⁹ is an optionally substituted C₁-C₃ heteroalkyl. In some embodiments, R¹⁹ is hydrogen, oxo, acyl, hydroxyl, hydroxyalkyl, cyan, halogen, or C₁-C₆ alkyl. In some embodiments, R¹⁹ is optionally substituted C₁-C₃ alkyl. In some embodiments, R¹⁹ is C₁-C₃ alkyl substituted with one or more of, hydroxyl, cyano, or halogen. In some embodiments, R¹⁹ is optionally substituted —(CH₂)₀₋₂C₆-C₁₀ aryl. In some embodiments. R¹⁹ is —(CH₂)phenyl. In some embodiments, R¹⁹ is optionally substituted —(CH₂)phenyl. In some embodiments. R¹⁹ is C₆-C₁₀ aryl. In some embodiments, R¹⁹ is phenyl. In some embodiments, R¹⁹ is C₂-C₉ heteroalkyl. In some embodiments, R¹⁹ is C₃-C₇ cycloalkyl. In some embodiments, R¹⁹ is cyclopropyl, cyclobutyl, cyclopentyl, or cyclohexyl. In some embodiments, R¹⁹ is C₂-C₇ heterocycloalkyl. In some embodiments, R¹⁹ is pyrrolidinyl, piperidinyl, piperazinyl, tetrahydrothiophene, tetrahydrofuranyl, pyranyl, or morpholino.

In some embodiments of Formula (III), Formula (IIIa), Formula (IIIb), Formula (IIIc), or Formula (X-III), X is C(═O).

In some embodiments of Formula (III), Formula (IIIa), Formula (IIIb), Formula (IIIc), or Formula (X-III), L¹ is a bond.

In some embodiments of Formula (III), Formula (IIIa), Formula (IIIb), Formula (IIIc), or Formula (X-III). L² is a bond, O, S or NR¹⁵. In some embodiments, L² is 0.

In some embodiments of Formula (III), Formula (IIIa), Formula (IIIb), Formula (IIIc), or Formula (X-III), each R¹³ is independently OH, halogen, or C₁-C₃ alkyl.

In some embodiments of Formula (III), Formula (IIIa), Formula (IIIb), Formula (IIIc), or Formula (X-III), m is 0 or 1. In some embodiments, m is 0.

In some embodiments of Formula (III), Formula (IIIa), Formula (IIIb), Formula (IIIc), or Formula (X-III). E is

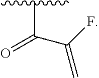 [SMILES: *C(=O)C(=C)F]

In some embodiments, E is

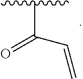 [SMILES: *C(=O)C=C]

In some embodiments, the radiolabeled compound comprises a structure of Formula (IIIa), or a salt or solvate thereof,

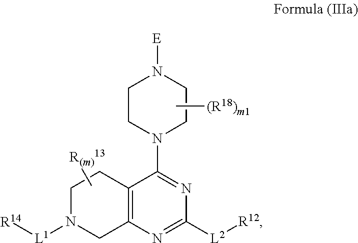

wherein m1 is 0, 1, 2, or 3; and
each R¹⁸ is independently halogen, oxo, C₁-C₆ alkyl, C₁-C₆ aminoalkyl, C₁-C₆ haloalkyl, C₂-C₆ alkenyl, C₂-C₆ alkynyl, C₁-C₆ heteroalkyl, —CN, —C(O)OR¹⁵, —C(O)N(R¹⁵)(R^{15′}), —N(R¹⁵)(R^{15′}), or OR¹⁵, and wherein each of the alkyl, aminoalkyl, haloalkyl, alkenyl, alkynyl, or heteroalkyl is optionally substituted.

In some embodiments, the radiolabeled compound comprises a structure listed in Table 4A.

In one aspect, provided herein is a radiolabeled compound comprising:
(a) a structure of Formula (III), or a salt or solvate thereof,
wherein

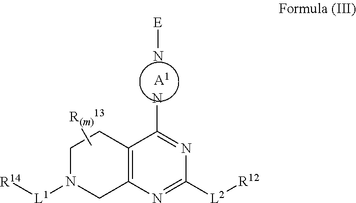

ring Q¹ is a 4-12 membered saturated or partially saturated monocyclic or bicyclic ring, wherein the monocyclic or bicyclic ring is optionally substituted;
   L¹ is a bond, —C(═O)—, O, S. NR¹⁵, optionally substituted C₁-C₃ alkylene, optionally substituted C₁-C₃ heteroalkylene;
   L² is a bond, —C(═O)—, O, S or NR;
E is

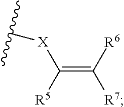

X is C(═O), P(═O)OR², C(═S), or S(═O)ₙ, where n is 1 or 2;
R² is H or optionally substituted C₁-C₃ alkyl;
R⁵ is hydrogen, cyan, halogen, C₁-C₆ alkyl, C₁-C₆ alkoxyl, C₁-C₆ heteroalkyl, C₃-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆, alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆ alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R¹² is hydrogen, C₁-C₆ alkyl, C₁-C₆ heteroalkyl, -L³-NR¹⁵R^{15′}, heterocycloalkyl, -L³-heterocycloalkyl, cycloalkyl, -L-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted; each R¹³ is independently OH, halogen, oxo, substituted or unsubstituted C₁-C₆ alkyl, or substituted or unsubstituted C₁-C₆ heteroalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or optionally substituted C₁-C₃ alkyl;
each R^{15′} is independently hydrogen, acyl, optionally substituted C₁-C₃ alkyl, optionally substituted C₁-C₃ heteroalkyl or optionally substituted C₁-C₃ hydroxyalkyl; and
m is 0, 1, or 2;
(b) a covalently bonded radioisotope R*; and
(c) a linker covalently connecting the radioisotope R* and the structure of Formula (III) or a salt or solvate thereof,
In some embodiments, R* is selected from a radioisotope in Table 6C or Table 6D.

In some embodiments, the radiolabeled compound comprises a structure of Formula (IIIb),

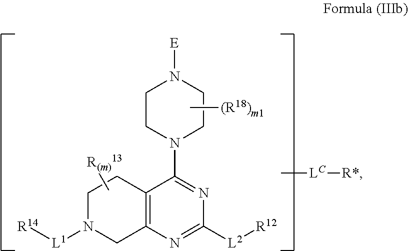

wherein
L^{C} is a linker comprising 1 to 20 groups independently selected from —CR^{b}R^{b}—, —C(═O)—, —S(═O)—, —S(═O)₂—NR^{a}—

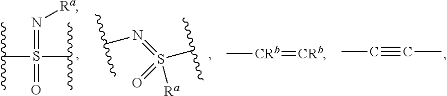

—O—, —S—, —C(═O)O—, —OC═O—, —C═O—, NR^{a}—, —NR^{a}C(═O)—, —S(═O)₂NR^{a}—, —NR^{a}C(═O)₂—, —NR^{a}C(═O)NR^{a}—, —NR^{a}C(═O)O—, —OC(═O)NR^{a}—, arylene, heteroarylene;
each R^{a} is independently hydrogen, halogen, C₁-C₆alkyl, C₁-C₆haloalkyl, C₁-C₆hydroxyalkyl. C₁-C₆aminoalkyl, C₁-C₆heteroalkyl, C₂-C₆alkenyl, C₂-C₆alkynyl, C₃-C₆cycloalkyl, C₂-C₉heterocycloalkyl, aryl, or heteroaryl;
each R^{b} is independently hydrogen, halogen, —CN, —NO₂, —OR^{a}, —SR^{a}, C₁-C₆alkyl, C₁-C₆haloalkyl, C₁-C₆hydroxyalkyl, C₂-C₅ aminoalkyl, C₁-C₅heteroalkyl, C₂-C₆alkenyl, C₂-C₆alkynyl, C₃-C₈cycloalkyl, C₂-C₉heterocycloalkyl, aryl, or heteroaryl;
   each R¹⁸ is independently halogen, oxo, C₁-C₆ alkyl, C₁-C₆, aminoalkyl, C₁-C₆ haloalkyl, C₂-C₆ alkenyl, C₂-C₆ alkynyl, C₁-C₆ heteroalkyl, —CN, —C(O)OR¹⁵, —C(O)N(R¹⁰)(R^{10′}), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₁-C₆haloalkyl, C₁-C₆ alkoxy, C₃-C₆cycloalkyl, and C₂-C₅heterocycloalkyl;
   R¹² is C₁-C₆, alkyl, C₁-C₆ heteroalkyl, -L³—NR¹⁵R^{15′}, C₂-C₁₂heterocycloalkyl, -L³-C₂-C₁₂ heterocycloalkyl, C₃-C₁₅cycloalkyl, -L³-C₃-C₁₅cycloalkyl, aryl, C₁-C₉ heteroaryl, -L³-aryl, or L³-C₁-C₉ heteroaryl, wherein each of the heterocycloalkyl, cycloalkyl, aryl, heteroaryl, alkyl or heteroalkyl is optionally substituted with one, two, three or four groups selected from R¹⁹:
m1 is 0, 1, 2, or 3; and
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At); and
the other variables are as defined in Formula (III), Formula (IIIa), Formula (IIIa-1) or Formula (IIIa-2).

In some embodiments, the radiolabeled compound comprises a structure of Formula (IIIc).

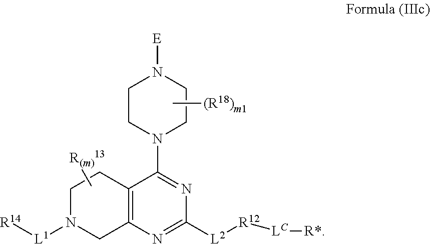

Compounds of Formula (III), Formula (IIIa), Formula (IIIa-1), Formula (IIIa-2), Formula (IIIb), and Formula (IIIc) including pharmaceutically acceptable salts, prodrugs, active metabolites, and pharmaceutically acceptable solvates thereof, can form a covalent bond with KRAS G12C.

In some embodiments of Formula (III), Formula (IIIa), Formula (IIIa-1), Formula (IIIa-2). Formula (IIIb), or Formula (IIIc), the radiolabeled compound comprises a structure listed in Table 4B.

In some embodiments of Formula (IIIb) or Formula (IIIc), L^{C} is a linker as described herein.

In some embodiments of Formula (IIIb) or Formula (IIIc), L^{C} comprises a structure of Table 3C. In some embodiments, L^{C} comprises one or more structures of Table 3A and 3B.

In some embodiments of Formula (IIIb) or Formula (IIIc). L^{C} comprises

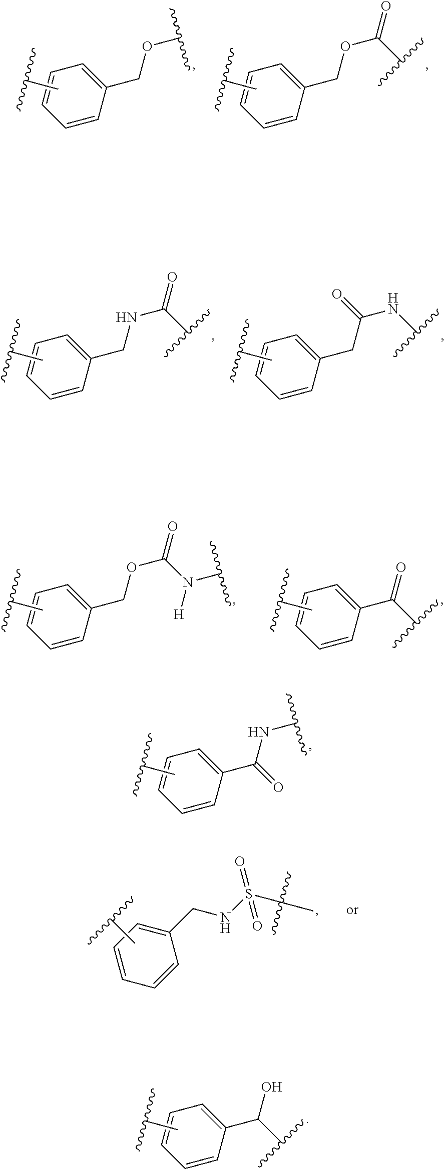

In some embodiments of Formula (IIIb) or Formula (IIIc), L^{C} is selected from the group consisting of

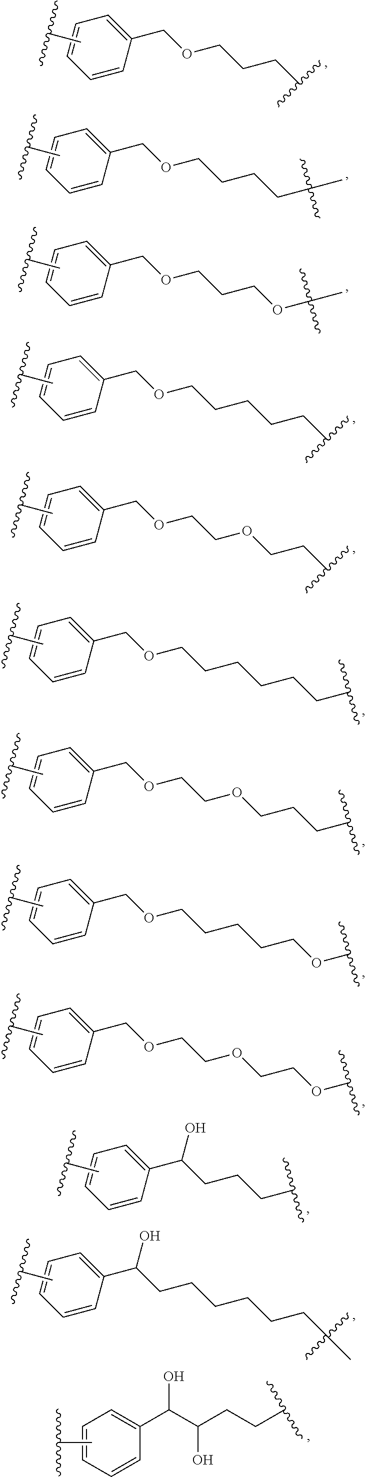

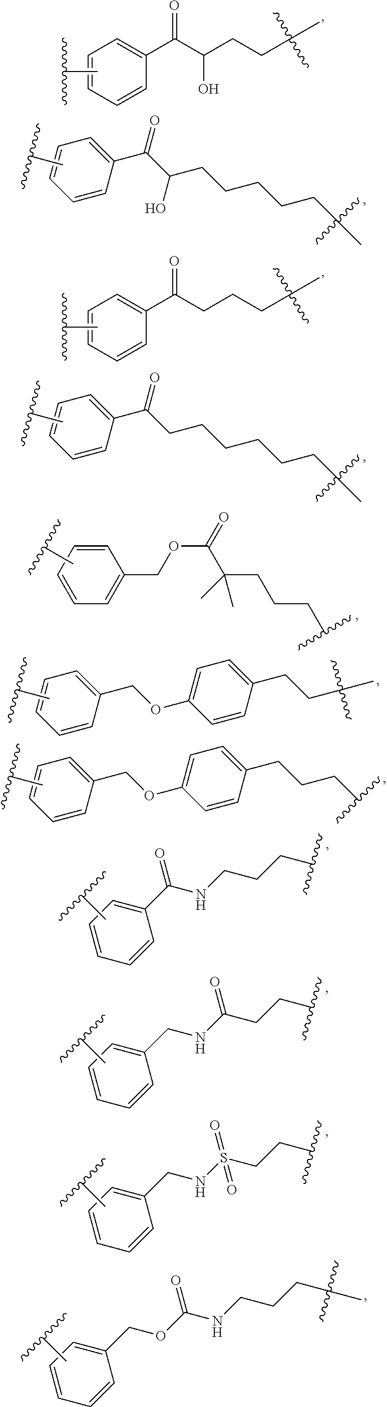

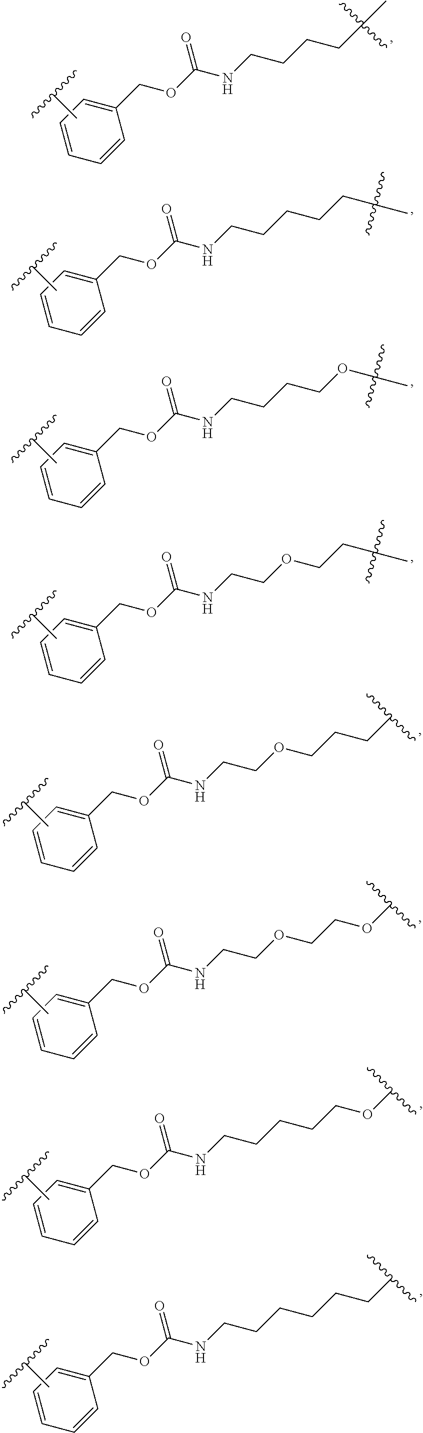

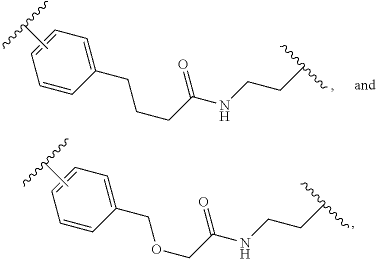

wherein R* is connected to the phenylene.

In some embodiments, the radiolabeled compound comprises a structure of Formula (IIIb) or Formula (IIIc) wherein L^{C}-R* is

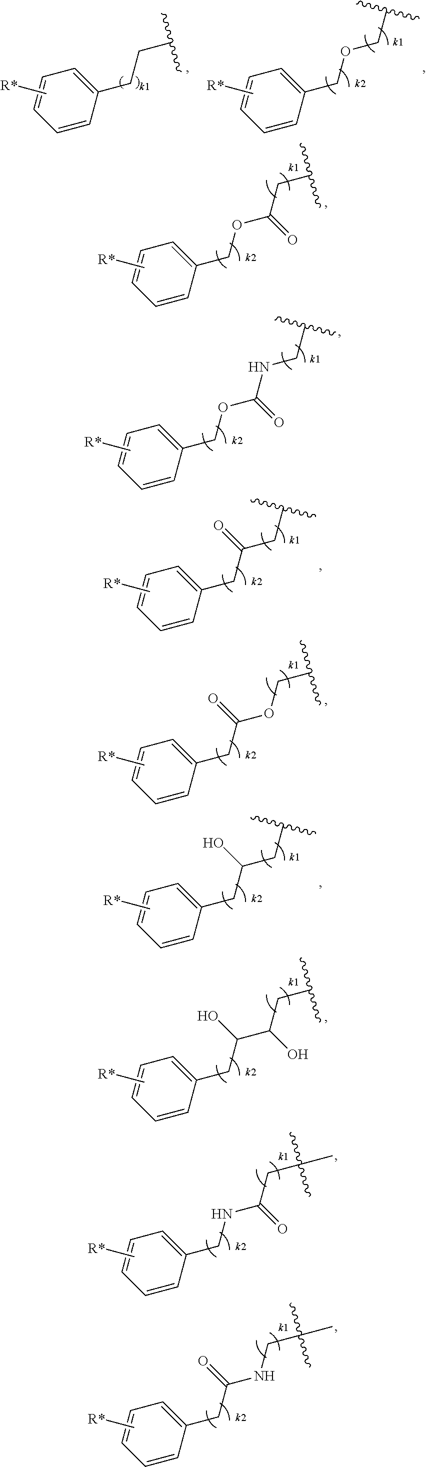

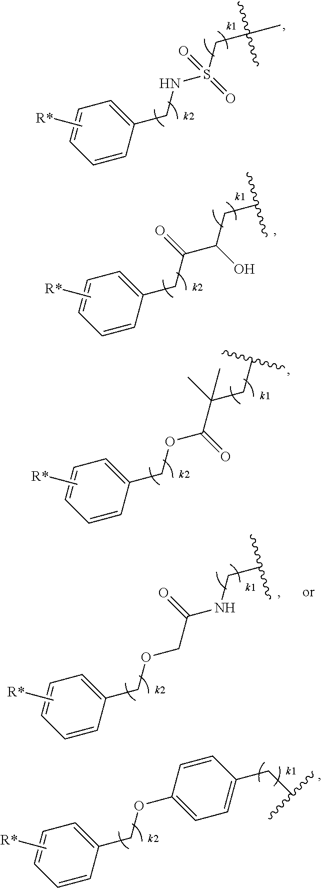

wherein each k1 and k2 is independently 0 or an integer selected from 1 to 10. In some embodiments, each k1 and k2 is independently 0 or an integer selected from 1 to 5. In some embodiments, k1 is 0 to 5 and k2 is 0 to 2. In some embodiments, k1 is 2 to 4 and k2 is 0 to 1.

In some embodiments, the radiolabeled compound comprises a structure of Formula (IIIb) or Formula (IIIc) wherein L^{C}-R* is

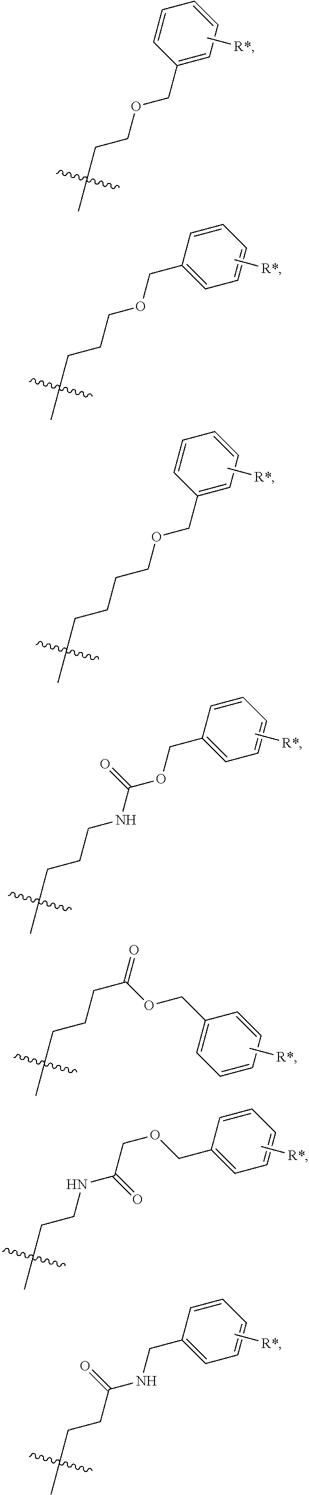

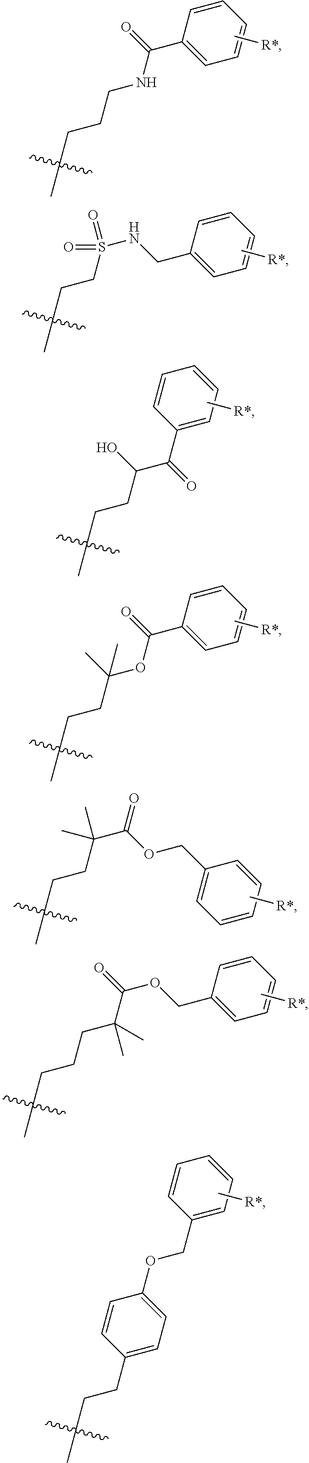

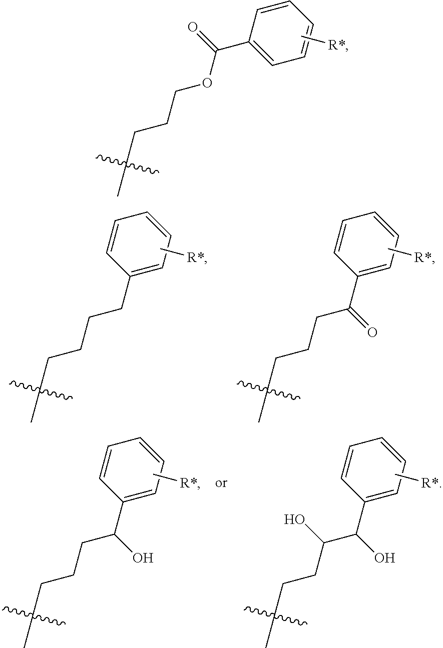

In some embodiments, R* is selected from a radioisotope in Table 6C or Table 6D. In some embodiments, R* is iodine-131 (I) or astatine-211 (²¹¹At). In some embodiments, provided herein are compounds having the structures of the radiolabeled compounds described herein (e.g., a compound of Formula (III), Formula (IIIa), Formula (IIIa-1), Formula (IIIa-2), Formula (IIIb), or Formula (IIIc), except that the radioisotope is replaced with a surrogate (e.g., ¹³¹I replaced with iodine). i.e., a cold compound. In some embodiments, a radionuclide of the radiolabeled compounds described herein can be replaced with a surrogate (e.g., ¹³¹I replaced with iodine) for testing and experimental purposes.

In one aspect, provided herein is a radiolabeled compound has a structure of Formula (IV), or a salt or solvate thereof,

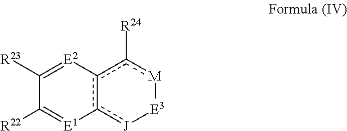

wherein
E¹ and E² are each independently N or CR²¹;
E³ is CR²⁹R²⁹, C═CR²⁸R²⁹, C═O, C═S, or C═NR²⁸;
J is N, NR³⁰, or CR³⁰;
M is N, NR³³, or CR³³;
[symbol] is a single or double bond as necessary to give every atom its normal valence;
R²¹ is independently H, hydroxyl, cyano, halogen, C₁-C₆ alkyl, C₁-C₆ alkoxyl, or C₁-C₆ heteroalkyl, wherein each of the alkyl, alkoxy, and heteroalkyl is optionally substituted;
R²² is hydrogen, halogen, C₁-C₆ alkyl, C₂-C₆ alkenyl, C₂-C₆ alkynyl, OR^{22′}, N(R^{22′})₂, cycloalkyl, —C₁-C₃ alkylene-cycloalkyl, heterocycloalkyl, —C₁-C₆alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted, and each R^{22′} is independently H, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form a 3-7-membered ring;
R²³ is hydrogen, halogen, C₁-C₆ alkyl, C₁-C₆ alkoxy, C₂-C₆ alkenyl, C₂-C₆ alkynyl, cycloalkyl, heterocycloalkyl, aryl, heteroaryl, -L⁴-cycloalkyl, -L⁴-heterocycloalkyl, -L⁴-aryl, or -L⁴-heteroaryl, wherein each of the alkyl, alkoxy, cycloalkyl, heterocycloalkyl, alkenyl, alkynyl, aryl, and heteroaryl is optionally substituted;
L⁴ is C₁-C₄ alkylene or C₁-C₄ heteroalkylene, each of which is optionally substituted;
R²⁴ is

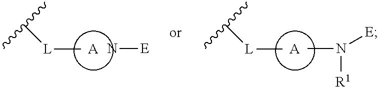

ring A is an optionally substituted 4-7 membered monocyclic ring or optionally substituted 6-11 membered bicyclic, bridged, fused, or spiro ring;
R¹ is H, optionally substituted C₁-C₆ alkyl, optionally substituted C₁-C₆ heteroalkyl, optionally substituted C₃-C₆ cycloalkyl, or optionally substituted C₂-C₅ heterocycloalkyl;
L is a bond, S, O, or NR^{10′}, optionally substituted C₁-C₆ alkylene, or optionally substituted C₁-C₆ heteroalkylene;
E represents a structure of Formula (Ic),

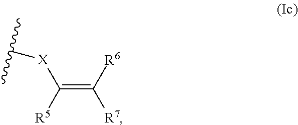

wherein,
X is C(═O), P(═O)OR², C(═S), or S(═O)ₙ, where n is 1 or 2;
R² is H or optionally substituted C₁-C₃ alkyl;
R⁵ is hydrogen, cyano, halogen, C₁-C₆ alkyl, C₁-C₆ alkoxyl, C₁-C₆ heteroalkyl, C₁-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₁-C₆ cycloalkyl, C₂-C₅ heterocycloalkyl, heteroaryl, —C₁-C₆ alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently hydrogen, hydroxyl, C₁-C₆ alkyl, C₁-C₆ alkoxy, cyan, nitro, C₃-C₆ cycloalkyl, or C₂-C₆ heterocycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, -L³-cycloalkyl, -L³-heterocycloalkyl, -L⁴-aryl, -L³-heteroaryl, —OR¹⁰, —SR¹⁰, —N(R¹⁰)(R^{10′}), —C(O)OR¹⁰, —OC(O)N(R¹⁰)(R^{10′}), —N(R^{10′})C(O)N(R¹⁰)(R^{10′}), —N(R¹⁰)C(O)OR¹⁵, —N(R^{10′})S(O)₂R¹¹, —C(O)R¹¹, —S(O)R¹¹, —OC(═O)R¹¹, —C(O)N(R¹⁰)(R^{10′}), —C(O)C(O)N(R¹⁰)(R^{10′}), —N(R^{10′})C(O)R¹⁰. —S(O)R¹¹, —S(O)₂N(R¹⁰)(R^{10′})—, S(═O)(═NH)N(R¹⁰)(R^{10′}), —CH₂C(O)N(R¹⁰)(R^{10′}), —CH₂N(R¹⁰)C(O)R¹¹, —CH₂S(O)₂R¹¹, or —CH₂S(O)₂N(R¹⁰)(R^{10′}), wherein each of the alkyl, heteroalkyl, cycloalkyl, heterocycloalkyl, aryl, and heteroaryl is optionally substituted;
R³³ is H, C₁-C₆ alkyl, C₁-C₆ heteroalkyl, or C₃—C cycloalkyl, wherein each of the alkyl, heteroalkyl, and cycloalkyl is optionally substituted;
each R¹⁰ is independently selected from hydrogen, C₁-C₆ alkyl, C₁-C₆haloalkyl, C₂-C₆ alkenyl, C₂-C₆ alkynyl, C₃-C₆ cycloalkyl, C₂-C₉heterocycloalkyl, C₁-C₁₀ aryl, and C₁-C₆ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉ heteroaryl;
each R^{10′} is independently selected from hydrogen, C₁-C₆ alkyl, and C₁-C₆haloalkyl; and
each R¹¹ is independently selected C₁-C₆ alkyl, C₂-C₆ alkenyl, C₂-C₆ alkynyl, C₃-C₆ cycloalkyl, C₂-C₉ heterocycloalkyl, C₆-C₁₀ aryl, and C₁-C₉ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₅ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉heteroaryl;
provided that at least one of R²¹, R²², R²³, R²⁴, and R³⁰ comprises a covalently bonded radioisotope R.

In some embodiments, R* is selected from a radioisotope in Table 6C or Table 6D.

In one aspect, described herein is a radiopharmaceutical conjugate comprising a) a targeting ligand that is configured to form a covalent bond with a KRAS protein at the G12C position, wherein the residue position numbering of the KRAS protein is based on SEQ ID NO: 1 or SEQ ID NO: 2 and b) a radionuclide. In some embodiments, the targeting ligand comprises a structure of Formula (IV), or a salt or solvate thereof,

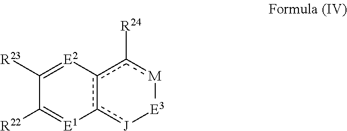

wherein
E¹ and E² are each independently N or CR²¹;
E³ is CR²⁸R²⁹, C═CR²⁸R²⁹, C═O, C═S, or C═NR²⁸;
J is N, NR³⁰, or CR³⁰;
M is N, NR³³, or CR³³;
[symbol] is a single or double bond as necessary to give every atom its normal valence;
R²¹ is independently hydrogen, hydroxyl, cyano, halogen, C₁-C₆ alkyl, C₁-C₆ alkoxyl, or C₁-C₆ heteroalkyl, wherein each of the alkyl, alkoxy, and heteroalkyl are optionally substituted;
R²² is hydrogen, halogen, C₁-C₆ alkyl, C₂-C₆ alkenyl, C₂-C₆ alkynyl, OR^{22′}, N(R²²)₂, cycloalkyl, heterocycloalkyl, aryl, or heteroaryl, wherein each of the alkyl, alkylene, alkenyl, alkynyl, cycloalkyl, heterocycloalkyl, aryl, and heteroaryl is optionally substituted, and each R is independently hydrogen, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form a 3-7-membered ring;
R²³ is hydrogen, halogen, C₂-C₅ alkyl, C₁-C₆ alkoxy, C₂-C₆ alkenyl, C₂-C₆ alkynyl, C₂-C₆alkynyl, heterocycloalkyl, aryl, heteroaryl, wherein each of the alkyl, alkoxy, cycloalkyl, heterocycloalkyl, alkenyl, alkynyl, aryl, and heteroaryl is optionally substituted;
R²⁴ is

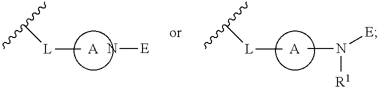

ring A is an optionally substituted 4-7 membered monocyclic ring of optionally substituted 6-11 membered bicyclic, bridged, fused, or spiro ring;
R¹ is hydrogen, optionally substituted C₁-C₆ alkyl, optionally substituted C₁-C₆ heteroalkyl, optionally substituted C₃-C₆ cycloalkyl, or optionally substituted C₂-C₆ heterocycloalkyl;
L is a bond, C₁-C₆ alkylene, C₁-C₆ heteroalkylene, S, O, or NH;
E represents a structure of Formula (Ic),

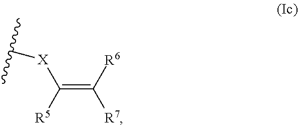

wherein,
X is C(═C)), P(═O)OR², C(═S), or S(═O)ₙ, where n is 1 or 2.
R² is hydrogen or optionally substituted C₁-C₃ alkyl;
R⁵ is hydrogen, cyano, halogen, C₁-C₆; alkyl, C₁-C₃ alkoxyl, C₁-C₆ heteroalkyl, C₃-G; cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyan, halogen, C₁-C₆ alkyl, C₁-C₆ alkoxyl, C₁-C₆ heteroalkyl, C₃-C₆ cycloalkyl, C₂-C₇heterocycloalkyl, heteroaryl, —C₁-C₆alkylene-heteoraryl, —C₁-C₆alkylene-NH(C₂-C₅ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆; alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently hydrogen, hydroxyl, C₁-C₆ alkyl, C₁-C₆ alkoxy, cyano, nitro, or C₃-C₆ cycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, —C₁-C₆alkylene-cycloalkyl, —C₁-C₆alkylene-heterocycloalkyl, —C₁-C₆alkylene-aryl, —C₁-C₆alkylene-heteroaryl, —C₁-C₆heteroalkylene-cycloalkyl, —C₁-C₆heteroalkylene-heterocycloalkyl, —C₁-C₆heteroalkylene-aryl, —C₁-C₆alkylene-heteroaryl wherein each of the alkyl, alkoxy, heteroalkyl, cycloalkyl, heterocycloalkyl, aryl, and heteroaryl is optionally substituted; and
R³³ is hydrogen, C₁-C₆ alkyl, C₁-C₆ heteroalkyl, or C₃-C₆ cycloalkyl, wherein each of the alkyl, heteroalkyl, and cycloalkyl is optionally substituted.
In some embodiments, the structure of Formula (IV) is attached to the rest of the conjugate at any suitable position.

In some embodiments, the radionuclide is covalently bound to the structure of Formula (IV). In some embodiments, the structure of Formula (IV) is attached to the linker or to the rest of the conjugate via group J or group E³. In some embodiments, J is NR³⁰, and the structure of Formula (IV) is attached to the linker or to the rest of the conjugate via group R³⁰. In some embodiments, the structure of Formula (IV) is attached to the linker or to the rest of the conjugate via group R.

In some embodiments of Formula (IV),
E¹ and E² are each independently N or CR²¹;
E³ is CR²⁸R²⁹, C═CR²⁸R²⁹, C═O. C═S, or C═NR¹⁵;
J is N, NR³⁰, or CR³⁰;
M is N, NR³³, or CR³³;
[symbol] is a single or double bond as necessary to give every atom its normal valence;
R²¹ is independently hydrogen, hydroxyl, cyano, halogen, C₁-C₆ alkyl, C₁-C₆ alkoxyl, or C₁-C₆ heteroalkyl, wherein each of the alkyl, alkoxy, and heteroalkyl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, C₂-C₅ heterocycloalkyl, C₆-C₁₀ aryl, and C₁-C₉ heteroaryl;
R²² is hydrogen, halogen, C₁-C₆ alkyl, C₂-C₆ alkenyl, C₂-C₆ alkynyl, OR^{22′}, N(R^{22′})₂, cycloalkyl, heterocycloalkyl, aryl, or heteroaryl, wherein each of the alkyl, alkylene, alkenyl, alkynyl, cycloalkyl, heterocycloalkyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, and C₂-C₅heterocycloalkyl, and each R²² is independently hydrogen, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted with one, two, or three groups selected from halogen, —CN, —NO₂, amino, hydroxy, C₁-C₆ alkyl, C₁-C₆haloalkyl, C₁-C₆ alkoxy, C₃-C₆ cycloalkyl, C₂-C₆ heterocycloalkyl, C₆-C₁₀ aryl, and C₁-C₉ heteroaryl; or two together with the nitrogen atom to which they are attached, form an optionally substituted 3-7-membered ring with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉heteroaryl,
R²³ is hydrogen, halogen, C₁-C₆ alkyl, C₁-C₆ alkoxy, C₂-C₆ alkenyl, C₂-C₆ alkynyl, cycloalkyl, heterocycloalkyl, aryl, heteroaryl, wherein each of the alkyl, alkoxy, cycloalkyl, heterocycloalkyl, alkenyl, alkynyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉heteroaryl;
R²⁴ is

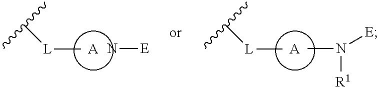

ring A is an optionally substituted 4-7 membered monocyclic ring or optionally substituted 6-11 membered bicyclic, bridged, fused, or spiro ring each of which is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, C₂-C₅ heterocycloalkyl, C₆-C₁₀ aryl, and C₁-C₉ heteroaryl;
R¹ is hydrogen, optionally substituted C₁-C₆ alkyl, optionally substituted C₁-C₆ heteroalkyl, optionally substituted C₃-C₆ cycloalkyl, or optionally substituted C₂-C₆ heterocycloalkyl each of which is optionally substituted with one, two, or three groups selected from halogen, —CN, —NO₂, amino, hydroxy, C₁-C₆ alkyl, C₁-C₆ haloalkyl, C₁-C₆ alkoxy, C₁-C₆cycloalkyl, C₁-C₅heterocycloalkyl, C₆-C₁₀ aryl, and C₁-C₉heteroaryl;
L is a bond, C₁-C₆ alkylene, C₁-C₆ heteroalkylene, S, O, or NH;
E represents a structure of Formula (Ic),

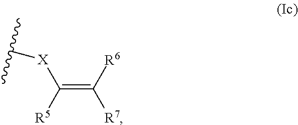

wherein,
X is C(═C)), P(═O)OR², C(═S), or S(═O)ₙ, where n is 1 or 2.
R² is hydrogen or substituted or unsubstituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₅ heterocycloalkyl, C₁-C₁₀ aryl, and C₁-C₉heteroaryl;
R⁵ is hydrogen, cyano, halogen, C₁-C₆ alkyl, C₁-C₆ alkoxyl, C₁-C₆heteroalkyl, C₁-C₆ cycloalkyl, or C₂-C₆ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₆ heterocycloalkyl, C₆-C₁₀ aryl, and C₁-C₉ heteroaryl;
R⁷ is hydrogen, cyan, halogen, C₁-C₆alkyl, C₁-C₆ alkoxyl, C₁-C₆ heteroalkyl, C₃-C₆ cycloalkyl, C₂-C₇heterocycloalkyl, heteroaryl, —C₁-C₆alkylene-heteoraryl, —C₁-C₆alkylene-NH(C₂-C₅ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉heteroaryl; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyan, halogen, C₁-C₆ alkyl, C₁-C₆ alkoxyl, C₁-C₆ heteroalkyl, C₃-C₆ cycloalkyl, C—C heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₅ heterocycloalkyl, C₆-C₁₀ aryl, and C₁-C₉heteroaryl,
each R¹⁷ is independently halogen, hydroxyl, C₁-C₆ alkyl, C₃-C₆, cycloalkyl, C₁-C₆ alkoxy, C₁-C₆ haloalkyl, amino, cyano, C₁-C₆ heteroalkyl, C₁-C₆ hydroxyalkyl, —O—C₁-C₆haloalkyl, or —S—C₁-C₆haloalkyl.
R²⁸ and R²⁹ are each independently hydrogen, hydroxyl, C₁-C₆ alkyl, C₁-C₆ alkoxy, cyan, nitro, or C₃-C₆ cycloalkyl, or R⁵ and R⁷ taken together with the carbon atom to which they are attached form a 3-6 membered ring;
R³⁰ is selected from halogen, cyan, C₁-C₆ alkyl, C₁-C₆ alkoxy, C₁-C₆ heteroalkyl, cycloalkyl, heterocycloalkyl, aryl, heteroaryl, —C₁-C₆alkylene-cycloalkyl, —C₁-C₆alkylene-heterocycloalkyl, —C₂-C₅ alkylene-aryl, —C₁-C₆alkylene-heteroaryl, —C₁-C₆heteroalkylene-cycloalkyl, —C₁-C₆heteroalkylene-heterocycloalkyl, —C₁-C₆heteroalkylene-aryl, —C₁-C₆alkylene-heteroaryl wherein each of the alkyl, alkoxy, heteroalkyl, cycloalkyl, heterocycloalkyl, aryl, and heteroaryl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₆ heterocycloalkyl, C₆-C₁₀ aryl, and C₁-C₉ heteroaryl; and
R³³ is hydrogen, C₁-C₆ alkyl, C₁-C₆ heteroalkyl, or C₃-C₆ cycloalkyl, wherein each of the alkyl, heteroalkyl, and cycloalkyl is optionally substituted with one, two, or three groups selected from halogen, —CN, hydroxy, C₁-C₆ alkyl, C₁-C₆ haloalkyl, C₁-C₆ alkoxy, C₁-C₆ cycloalkyl, C₂-C₅ heterocycloalkyl, C₆-C₁₀ aryl, and C₁-C₉heteroaryl.

In some embodiments of Formula (IV), Formula (IVa), or Formula (X-IV), E¹ is N. In some embodiments, E¹ is CR²¹.

In some embodiments of Formula (IV), Formula (IVa), or Formula (X-IV), E² is N. In some embodiments, E² is CR²¹.

In some embodiments of Formula (IV) or Formula (X-IV), E³ is C═O, C═S, or C═NH. In some embodiments. E³ is C═O.

In some embodiments of Formula (IV) or Formula (X-IV), J is NR. In some embodiments, J is N. In some embodiments, J is CR³⁰.

In some embodiments of Formula (IV) or Formula (X-IV), M is N. In some embodiments, M is NR³³. In some embodiments, M is CR³³.

In some embodiments of Formula (IV) or Formula (X-IV), when J is NR³⁰, M is N or CR³³. In some embodiments, when M is NR³³, J is N or CR³⁰. In some embodiments, when J is CR³⁰, M is N or NR³³. In some embodiments, when M is CR³³, J is N or NR³⁰.

In some embodiments of Formula (IV) or Formula (X-IV), E¹ is N. E² is CR²¹; J is NR³⁰; and M is N. In some embodiments, E¹ is CR²¹; E² is CR²¹: J is NR³⁰; and M is N.

In some embodiments of Formula (IV), Formula (IVa), Formula (IVb), Formula (IVc), Formula (IVd), Formula (IVe), or Formula (X-IV), R²¹ is independently hydrogen, hydroxyl, cyano, halogen, C₁-C₆ alkyl, C₁-C₄haloalkyl, C₁-C₄alkoxyl, or C₁-C₄heteroalkyl. In some embodiments, R²¹ is independently hydrogen, hydroxyl, cyano, halogen, or methyl. In some embodiments, R²¹ is hydrogen.

In some embodiments of Formula (IV), Formula (IVa), Formula (IVb), Formula (IVc), or Formula (IVd), R²² is halogen, C₁-C₆alkyl, C₂-C₃alkenyl, C₂-C₃alkynyl, OR^{22′}, N(R^{22′})₂, C₃-C₆cycloalkyl, C₂-C₅heterocycloalkyl, C₆-C₁₄aryl, or C₂-C₁₄heteroaryl, each of the alkyl, alkenyl, alkynyl, cycloalkyl, heterocycloalkyl, aryl, and heteroaryl is optionally substituted, and each R^{22′} is independently hydrogen, C₁-C₆alkyl, C₃-C₆cycloalkyl, C₂-C₅heterocycloalkyl, C₂-C₃alkenyl, C₂-C₃alkynyl, C₆-C₁₄aryl, C₂-C₁₄heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form an optionally substituted 3-7-membered ring.

In some embodiments of Formula (IV), Formula (IVa), Formula (IVb), Formula (IVc), or Formula (IVd), R²² is C₀-C₃ alkylene-C₃-C₁₄ cycloalkyl, C₁-C₃ alkylene-C₂-C₁₄ heterocycloalkyl, C₀-C₃ alkylene-C₆-C₁₄ aryl, or C₀-C₃alkylene-C₂-C₁₄heteroaryl, each of which is optionally substituted. In some embodiments, R²² is hydrogen.

In some embodiments of Formula (IV), Formula (IVa), Formula (IVb), Formula (IVc), Formula (IVd), Formula (IVe), or Formula (X-IV), R²² is optionally substituted monocyclic cycloalkyl. In some embodiments, R²² is optionally substituted bicyclic cycloalkyl. In some embodiments, R²² is optionally substituted monocyclic heterocycloalkyl. In some embodiments, R²² is optionally substituted bicyclic heterocycloalkyl.

In some embodiments of Formula (IV), Formula (IVa), Formula (IVb), Formula (IVc), Formula (IVd), Formula (IVe), or Formula (X-IV). R²² is C₆-C₁₄aryl or C₂-C₁₂heteroaryl, each of which is optionally substituted. In some embodiments, R²² is aryl or heteroaryl substituted with one or more C₁-C₃alkyl, halogen, and/or hydroxyl. In some embodiments, R²² is C₆-C₁₀aryl optionally substituted. In some embodiments. R²² is optionally substituted phenyl. In some embodiments, R²² is optionally substituted naphthyl. In some embodiments, R²² is optionally substituted C₂-C₉ heteroaryl. In some embodiments, R²² is optionally substituted pyrrolyl, imidazolyl, pyridinyl, pyrazinyl, indolyl, or quinolinyl. In some embodiments, R²² is phenyl, optionally substituted with one or more C₁-C₃alkyl, halogen, and/or hydroxyl. In some embodiments, R²² is phenyl substituted with fluorine and hydroxyl.

In some embodiments of Formula (IV), Formula (IVa), Formula (IVb), Formula (IVc), Formula (IVd), Formula (IVe), or Formula (X-IV), R²² is optionally substituted with one or more substituents selected from: C₁-C₁₂ alkyl, C₁-C₁₂ heteroalkyl, C₂-C₁₂ alkenyl, C₂-C₁₂ alkynyl, C₅-C₂₀ aryl, C₅-C₂₀ heteroaryl, C₆-C₂₄ alkaryl, C₆-C₂₄ aralkyl, halo, hydroxyl, sulfhydryl, C₁-C₁₂ alkoxy, C₂-C₁₂ alkenyloxy, C₂-C₁₂alkynyloxy, C₅-C₂₀aryloxy, acyl (including C₂-C₂₄alkylcarbonyl (—CO-alkyl)), oxo, amino, —CN, isocyano, nitro, C₃-C₁₀cycloalkyl, and C₂-C₁₀heterocycloalkyl, each of which is optionally further substituted.

In some embodiments, of Formula (IV), Formula (IVa), Formula (IVb), Formula (IVc), Formula (IVd), Formula (IVe), or Formula (X-IV) is attached to the linker or to the rest of the conjugate via group R²². In some embodiments, of Formula (IV), Formula (IVa), Formula (IVb), Formula (IVc), Formula (IVd). Formula (IVc), or Formula (X-IV) is attached to the linker or to the rest of the conjugate via a substituent of group R²².

In some embodiments of Formula (IV), Formula (IVa), Formula (IVb), Formula (IVc), Formula (IVd), or Formula (IVe), R²² comprises the covalently bonded radioisotope R*. In some embodiments of Formula (IV), Formula (IVa), Formula (IVb), or Formula (IVc). R²² is

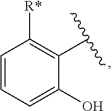

and R* is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, R* is ¹³¹I.

In some embodiments of Formula (IV), Formula (IVa), Formula (IVb), Formula (IVc), Formula (IVd), Formula (IVe), or Formula (X-IV), R²³ is hydrogen, halogen, C₁-C₆alkyl, C₁-C₃alkoxy, C₃-C₆cycloalkyl, C₂-C₅heterocycloalkyl, C₂-C₃alkenyl, C₂-C₃alkynyl, C₆-C₁₄aryl, or C₂-C₁₄heteroaryl, wherein each of the alkyl, alkoxy, cycloalkyl, heterocycloalkyl, alkenyl, alkynyl, aryl, and heteroaryl is optionally substituted. In some embodiments, R²³ is halogen, C₁-C₃alkoxy, or C₁-C₃alkyl where the C₁-C₃alkyl is optionally substituted with a halo group. In some embodiments, R²³ is hydrogen.

In some embodiments of Formula (IV), Formula (IVa), Formula (IVb), Formula (IVc), or Formula (IVd), R²³ comprises the radionuclide. In some embodiments, the radionuclide is covalently bound.

In some embodiments of Formula (IV), Formula (IVa), Formula (IVb), or Formula (IVc), R²³ is halogen and the halogen is the radionuclide selected from fluorine-18 (F), iodine-131 (¹³¹I), iodine-123 (¹²³I), iodine-124 (¹²⁴I), iodine-125 (¹²⁵I), and astatine-211 (²¹¹At). In some embodiments, R²³ is halogen and the halogen is a radioisotope selected from ¹²⁴I, ¹²⁵I, and ¹³¹I. In some embodiments, R²³ is ¹³¹I.

In some embodiments of Formula (IV), Formula (IVa), Formula (IVb), Formula (IVc), Formula (IVd), Formula (IVe), or Formula (X-IV), R²³ is C₃-C₆cycloalkyl or C₂-C₅heterocycloalkyl. In some embodiments. R²³ is cyclopropyl or cyclopentyl optionally substituted. In some embodiments, R²³ is cyclopropyl optionally substituted with hydroxyl, halo, or methyl groups. In some embodiments, R²³ is aziridinyl, pyrrolidinyl, tetrahydrothiophenyl, tetrahydrofuranyl each of which is optionally substituted.

In some embodiments of Formula (IV), Formula (IVa), Formula (IVb), Formula (IVc), Formula (IVd), Formula (IVe), or Formula (X-IV), R²³ is C₁-C₃haloalkyl. In some embodiments, R²³ is halogen. In some embodiments, R²³ is —CF₃. In some embodiments, R²³ is F.

In some embodiments of Formula (IV), Formula (IVa), Formula (IVb), Formula (IVc), Formula (IVd), Formula (IVe), or Formula (X-IV). R²³ is C₆-C₁₄aryl or C₂-C₁₄heteroaryl, each of which is optionally substituted. In some embodiments, R²³ is optionally substituted C₆-C₁₀aryl. In some embodiments, R²³ is optionally substituted phenyl. In some embodiments, R²³ is optionally substituted naphthyl. In some embodiments. R²³ is optionally substituted C₂-C₉ heteroaryl. In some embodiments, R²³ is optionally substituted pyrrolyl, imidazolyl, pyridinyl, pyrazinyl, indolyl, or quinolinyl. In some embodiments, R²³ is phenyl, optionally substituted with one or more C₁-C₃alkyl, halogen, and/or hydroxyl. In some embodiments. R²³ is phenyl substituted with fluorine and hydroxyl.

In some embodiments of Formula (IV) or Formula (X-IV), R²⁴ is

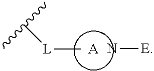

In some embodiments, R²¹ is

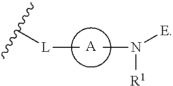

In some embodiments of Formula (IV), Formula (IVa), Formula (IVb), Formula (IVc), Formula (IVd), Formula (IVe), or Formula (X-IV), ring A is a substituted or unsubstituted 4-7 membered monocyclic ring. In some embodiments, ring A is substituted or unsubstituted 6 membered monocyclic heterocyclic ring. In some embodiments, ring A is piperazinyl substituted with halogen or C₁-C₃alkyl. In some embodiments, ring A is piperazinyl substituted with methyl. In some embodiments, ring A is unsubstituted piperazinyl.

In some embodiments of Formula (IV), Formula (IVa), Formula (IVb), Formula (IVc), Formula (IVd), Formula (IVc), or Formula (X-IV), L is L is a bond, C₁-C₆ alkylene, —O—CC₅ alkylene, —S—C₀-C₅ alkylene, or —NH—C₀-C₅ alkylene, and for C₂-C₅ alkylene, —O—C₂-C₅ alkylene, —S—C₂ C₅ alkylene, and NH—C₂-C₅ alkylene, one carbon atom of the alkylene group can optionally be replaced O. S, or NH.

In some embodiments of Formula (IV), Formula (IVa), Formula (IVb), Formula (IVc), Formula (IVd), Formula (IVe), or Formula (X-IV), L is a bond, C₁-C₃alkylene, S, O, or NH. In some embodiments, L is a bond, CH₂, O, or NH. In some embodiments, L is a bond.

In some embodiments of Formula (IV), Formula (IVa), Formula (IVb), Formula (IVc), Formula (IVd), Formula (IVe), or Formula (X-IV), X is C(═O).

In some embodiments of Formula (IV), Formula (IVa), Formula (IVb), Formula (IVc), Formula (IVd), Formula (IVe), or Formula (X-IV). R⁵ is hydrogen, cyano, halogen, C₁-C₆ alkyl, C₁-C₆ alkoxyl, C₁-C₆ heteroalkyl, C₃—C cycloalkyl, or C₁-C₅ heterocycloalkyl, each of which is optionally substituted. In some embodiments, R⁵ is hydrogen or a C₁-C₃alkyl optionally substituted by one or more hydroxyl and/or halogen. In some embodiments, R⁵ is a halogen. In some embodiments, R⁵ is fluoro. In some embodiments, R⁵ is C₁-C₆ heteroalkyl. In some embodiments, R⁵ is hydrogen. In some embodiments of Formula (IV). Formula (IVa), Formula (IVb), Formula (IVc), Formula (IVd), Formula (IVe), or Formula (X-IV), R⁷ is hydrogen, cyano, halogen, optionally substituted C₁-C₆ alkyl, optionally substituted C₁-C₆ alkoxyl, or optionally substituted C₁-C₆ heteroalkyl. In some embodiments, R⁷ is hydrogen. In some embodiments, R⁷ is C₁-C₆ heteroalkyl selected from —NHC(O)—C₁-C₃ alkyl and —CH₂NHC(O)—C₁-C₃ alkyl. In some embodiments of Formula (IV), Formula (IVa), Formula (IVb), Formula (IVc), Formula (IVd), Formula (IVe), or Formula (X-IV), R⁵ and R⁷ taken together with the carbon atoms to which they are attached form a 5-8 membered partially saturated cycloalkyl, wherein the cycloalkyl is optionally substituted with one or more R¹⁷, wherein each R¹⁷ is independently halogen, hydroxyl, C₁-C₆ alkyl, cycloalkyl, alkoxy, haloalkyl, amino, cyano, heteroalkyl, hydroxyalkyl, —O-haloalkyl, or —S-haloalkyl.

In some embodiments of Formula (IV), Formula (IVa), Formula (IVb), Formula (IVc), Formula (IVd), Formula (IVe), or Formula (X-IV), R⁶ is hydrogen, cyano, halogen, C₁-C₆ alkyl, C₁-C₆ alkoxyl, C₁-C₆ heteroalkyl, C₃-C₆ cycloalkyl, or C₂-C₆ heterocycloalkyl, each of which is optionally substituted. In some embodiments, R⁶ is hydrogen. In some embodiments, R⁶ is C₁-C₆ heteroalkyl selected from —NHC(O)—C₁-C₃ alkyl and —CH₂NHC(O)—C₁-C₆ alkyl. In some embodiments of Formula (IV) or Formula (X-IV), R²⁸ and R²⁹ are each independently hydrogen, C₁-C₃ alkyl, hydroxy, C₁-C₃alkoxy, cyano, nitro, or C₃-C₆ cycloalkyl.

In some embodiments of Formula (IV), Formula (IVa), Formula (IVb), Formula (IVc), Formula (IVd), Formula (IVe), or Formula (X-IV), R³⁰ is halogen, cyano, C₂-C₅ alkyl, C₃-C₆cycloalkyl, C₆-C₁₄aryl, C₁-C₈ heteroalkyl, C₂-C₁₄heterocycloalkyl, C₂-C₁₄heteroaryl, C₁-C₃alkyl-C₆-C₁₄aryl, C₁-C₃alkyl-C₃-C₁₄cycloalkyl, C₁-C₃alkyl₂-C₁₄heterocycloalkyl, C₁-C₃alkyl-C₂-C₁₄heteroaryl, C₂-C₅ alkoxy, C₀-C₃heteroalkyl-C₆-C₁₄aryl, C₀-C₃heteroalkyl-C₂-C₁₄heteroaryl, C₀-C₃heteroalkyl-C₃-C₆cycloalkyl, C₀-C₃heteroalkyl-C₂-C₁₄heterocycloalkyl, each of which is optionally substituted.

In some embodiments of Formula (IV), Formula (IVa), Formula (IVb), Formula (IVc), Formula (IVd), Formula (IVe), or Formula (X-IV), R³⁰ is C₁-C₈alkyl, C₀-C₃alkylene-C₆-C₁₄aryl, C₀-C₃alkylene-C₃-C₁₄ cycloalkyl, C₀-C₃ alkylene-C₂-C₁₄ heterocycloalkyl, C₀-C₃ alkylene-C₂-C₁₄ heteroaryl, C₁-C₆ alkoxy, C₀-C₃ alkylene-C₆-C₁₄ aryl, O—C₀-C₃ alkylene-C₆-C₁₄ heteroaryl, C₀-C₃ alkylene-C₃-C₁₄ cycloalkyl, C₀-C₃ alkylene-C₂-C₁₄ heterocycloalkyl, NH—C₁-C₈ alkyl, N(C₁-C₈ alkyl)₂. NH—C₀-C₃ alkylene-C₃-C₁₄ aryl, NH—C₀-C₃alkylene-C₂-C₆ 1 heteroaryl, NH—C₀-C₃alkylene-C₃-C₁₄ cycloalkyl. NH—C₀-C₃ alkylene-C₂-C₆heterocycloalkyl, halo, cyano, or C₁-C₆alkylene-amine.

In some embodiments, R³⁰ is C₆-C₁₀ aryl, optionally substituted with one or more of halogen, C₁-C₃alkyl, C₁-C₃alkoxyl, or cyano. In some embodiments, R³⁰ is a phenyl, optionally substituted with one or more of C₁-C₃alkoxyl, or cyano. In some embodiments, R³⁰ is C₂-C₁₄heteroaryl, optionally substituted with one or more of halogen, C₁-C₃alkyl, C₁-C₃alkoxyl, or cyano. In some embodiments, R³⁰ is a 6-membered heteroaryl, optionally substituted with one or more of C₁-C₃alkyl.

In some embodiments of Formula (IV) or Formula (X-IV), R³³ is hydrogen, C₁-C₆alkyl, C₁-C₆ haloalkyl, C₁-C₆alkylamine, or C₃-C₁₄ cycloalkyl. In some embodiments, R³³ is C₁-C₆ alkyl, C₁-C₆ haloalkyl, C₁-C₆alkylamine, or C₃-C₁₄cycloalkyl. In some embodiments, R³³ is C₁-C₆alkyl. In some embodiments, R³³ is methyl, ethyl, propyl, or isopropyl. In some embodiments, R³³ is C₃-C₆cycloalkyl. In some embodiments, R³³ is cyclopropyl or cyclopentyl. In some embodiments, R³³ is C₁-C₃ haloalkyl or C₁-C₃ alkylamine. In some embodiments, R³³ is hydrogen.

In some embodiments, the radiolabeled compound comprises a structure of Formula (IVa), or a salt or solvate thereof,

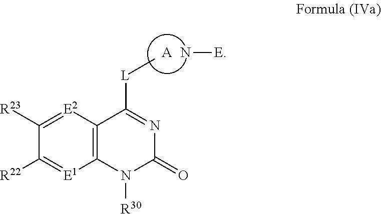

In some embodiments, the radiolabeled compound comprises a structure of Formula (IVb), or a salt or solvate thereof,

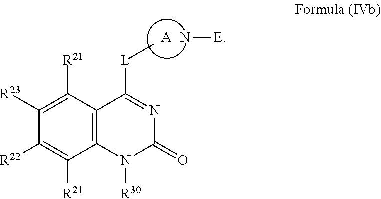

In some embodiments, the radiolabeled compound comprises a structure of Formula (IVc), or a salt or solvate thereof,

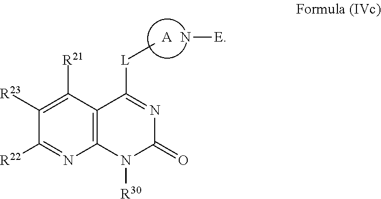

In some embodiments of Formula (IV), Formula (IVa), Formula (IVb), or Formula (IVc), the radiolabeled compound comprises a structure listed in Table 4C. In some embodiments, the radiolabeled compound has a structure listed in Table 4C.

In one aspect, provided herein is a radiolabeled compound, comprising (a) a structure of Formula (IV), or a salt or solvate thereof,
wherein

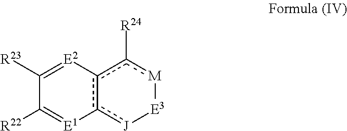

E¹ and E² are each independently N or CR²¹;
E³ is CR²⁸R²⁹, C═CR²⁸R²⁸, C═O, C═S, or C═NR²⁸;
J is N, NR³⁰, or CR³¹;
M is N, NR¹⁵, or CR³⁰;
[symbol] is a single or double bond as necessary to give every atom its normal valence;
R²¹ is independently hydrogen, hydroxyl, cyan, halogen, C₁-C₆ alkyl, C₁-C₆ alkoxyl, or C₁-C₆ heteroalkyl, wherein each of the alkyl, alkoxyl, and heteroalkyl is optionally substituted;
R²² is hydrogen, halogen, C₁-C₆ alkyl, C₂-C₆ alkenyl, C₂-C₆ alkynyl, OR^{22′}, N(R^{22′})₂, cycloalkyl, —C₁-C₃alkylene-cycloalkyl, heterocycloalkyl, —C₁-C₆alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted, and each R^{12′} is independently H, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form a 3-7-membered ring;
R²³ is hydrogen, halogen, C₁-C₆ alkyl, C₁-C₆ alkoxy, C₂-C₆ alkenyl, C₂-C₆ alkynyl, cycloalkyl, heterocycloalkyl, aryl, heteroaryl, -L⁴-cycloalkyl, -L³-heterocycloalkyl, -L⁴-aryl, or -L⁴-heteroaryl, wherein each of the alkyl, alkoxy, cycloalkyl, heterocycloalkyl, alkenyl, alkynyl, aryl, and heteroaryl is optionally substituted;
L⁴ is C₁-C₄ alkylene or C₁-C₄ heteroalkylene, each of which is optionally substituted;
R²⁴ is

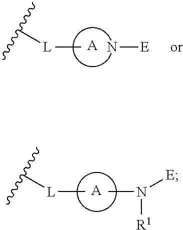

ring A is an optionally substituted 4-7 membered monocyclic ring or optionally substituted 6-11 membered bicyclic, bridged, fused, or spiro ring;
R¹ is H, optionally substituted C₁-C₆ alkyl, optionally substituted C₁-C₆ heteroalkyl, optionally substituted C₃-C₆ cycloalkyl, or optionally substituted C₂-C₆ heterocycloalkyl;
L is a bond, S, O, or NR^{10′}, optionally substituted C₁-C₆ alkylene, or optionally substituted C₁-C₆ heteroalkylene;
E represents a structure of Formula (Ic).

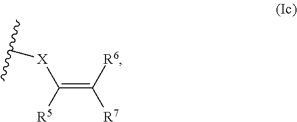

wherein,
X is C(═O), P(═O)OR², C(═S), or S(═O)ₙ, where n is 1 or 2;
R² is H or optionally substituted C₁-C₃ alkyl;
R⁵ is hydrogen, cyano, halogen, C₁-C₆ alkyl, C₁-C₆ alkoxyl, C₁-C₆ heteroalkyl, C₁-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₁-C₆ cycloalkyl, C₂-C₅ heterocycloalkyl, heteroaryl, —C₁-C₆ alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently hydrogen, hydroxyl, C₁-C₆ alkyl, C₁-C₆ alkoxy, cyan, nitro, C₃-C₆ cycloalkyl, or C₂-C₆ heterocycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, -L³-cycloalkyl, -L³-heterocycloalkyl, -L⁴-aryl, -L³-heteroaryl, —OR¹⁰, —SR¹⁰, —N(R¹⁰)(R^{10′}), —C(O)OR¹⁰, —OC(═O)N(R¹⁰)(R^{10′}), —N(R¹⁰)C(O)N(R¹⁰)(R^{10′}), —N(R¹⁰)C(O)OR¹¹, —N(R^{10′})S(O)₂R¹¹, —C(O)R¹¹, —S(O)R¹¹, —OC(═O)R¹¹, —C(O)N(R¹⁰)(R^{10′}), —C(O)C(O)N(R¹⁰)(R^{10′}), —N(R¹⁰)C(O)R¹¹, —S(O)R¹¹, —S(O)₂N(R¹⁰)(R^{10′})—, S(═O)(═NH)N(R¹⁰)(R^{10′}), —CH₂C(O)N(R¹⁰)(R^{10′}), —CH₂N(R¹⁰)C(O)R¹¹, —CH₂S(O)₂R¹¹, or —CH₂S(O)₂N(R¹⁰)(R^{10′}), wherein each of the alkyl, heteroalkyl, cycloalkyl, heterocycloalkyl, aryl, and heteroaryl is optionally substituted;
R³³ is H, C₁-C₆ alkyl, C₁-C₆ heteroalkyl, or C₃—C cycloalkyl, wherein each of the alkyl, heteroalkyl, and cycloalkyl is optionally substituted;
each R¹⁰ is independently selected from hydrogen, C₁-C₆ alkyl, C₁-C₆haloalkyl, C₂-C₆ alkenyl, C₂-C₆ alkynyl, C₃-C₆ cycloalkyl, C₂-C₉heterocycloalkyl, C₁-C₁₀ aryl, and C₁-C₆ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉ heteroaryl;
each R^{10′} is independently selected from hydrogen, C₁-C₆ alkyl, and C₁-C₆haloalkyl; and
each R¹¹ is independently selected C₁-C₆ alkyl, C₂-C₆ alkenyl, C₂-C₆ alkynyl, C₃-C₆ cycloalkyl, C₂-C₉ heterocycloalkyl, C₆-C₁₀ aryl, and C₁-C₅ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉heteroaryl;
(b) a covalently bonded radioisotope R*; and
(c) a linker covalently connecting the radioisotope R* and the structure of Formula (IV) or a salt or solvate thereof. In some embodiments, R* is selected from a radioisotope in Table 6C or Table 6D.

In some embodiments, the radiolabeled compound comprises a structure of Formula (IVd)

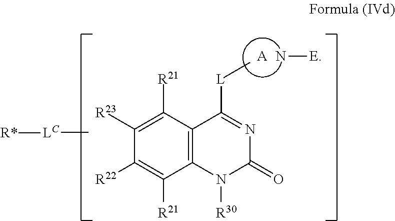

wherein
L^{C} is a linker comprising 1 to 20 groups independently selected from —CR^{b}R^{b}—, —C(═O)—, —S(═O)—, —S(═O)₂—, —NR^{a}—,

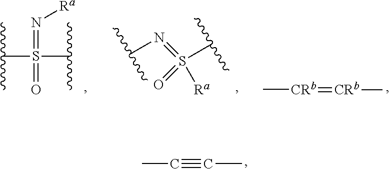

—O—, —S—, —C(═O)O—, —OC(═O)—, —C(═O)NR^{a}—, —NR^{a}C(═O)—, —S(═O)₂NR^{a}—, —NR^{a}S(═O)₂—, —NR^{a}C(═O)NR^{a}—, —NR^{a}C(═O)O—, —OC(═O)NR^{a}—, arylene, heteroarylene;
each R^{a} is independently hydrogen, halogen, C₁-C₆alkyl, C₁-C₄haloalkyl, C₁-C₆hydroxyalkyl, C₁-C₆aminoalkyl, C₁-C₆heteroalkyl, C₁-C₆alkenyl, C₃-C₆alkynyl, C₃-C₈cycloalkyl, C₂-C₉heterocycloalkyl, aryl, or heteroaryl;
each R^{b} is independently hydrogen, halogen, —CN, —NO₂, —OR^{a}, —SR^{a}, C₁-C₆alkyl, C₁-C₆haloalkyl, C₁-C₆hydroxyalkyl, C₁-C₆aminoalkyl, C₁-C₆ heteroalkyl, C₂-C₆alkenyl, C₂-C₆alkynyl, C₃-C₆cycloalkyl, C₂-C₉heterocycloalkyl, aryl, or heteroaryl;
R²² is C₁-C₆ alkyl, C₂-C₆ alkenyl, C₂-C₆ alkynyl, cycloalkyl, —C₁-C₃alkylene-cycloalkyl, heterocycloalkyl, —C₁-C₃alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted; and
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At).

In some embodiments, the radiolabeled compound comprises a structure of Formula (IVe),

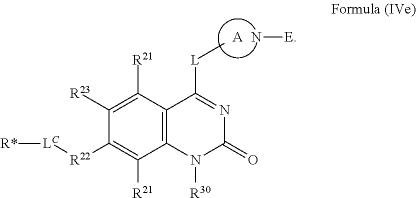

In some embodiments of Formula (IV), Formula (IVd), or Formula (IVe), the radiolabeled compound comprises a structure listed in Table 4D.

In some embodiments of Formula (IVd) or Formula (IVe), L^{C} is a linker as described herein.

In some embodiments of Formula (IVd) or Formula (IVe), L^{C} comprises a structure of Table 3C. In some embodiments, L^{C} comprises one or more structures of Table 3A and 3B.

In some embodiments of Formula (IVd) or Formula (IVc), L^{C} comprises

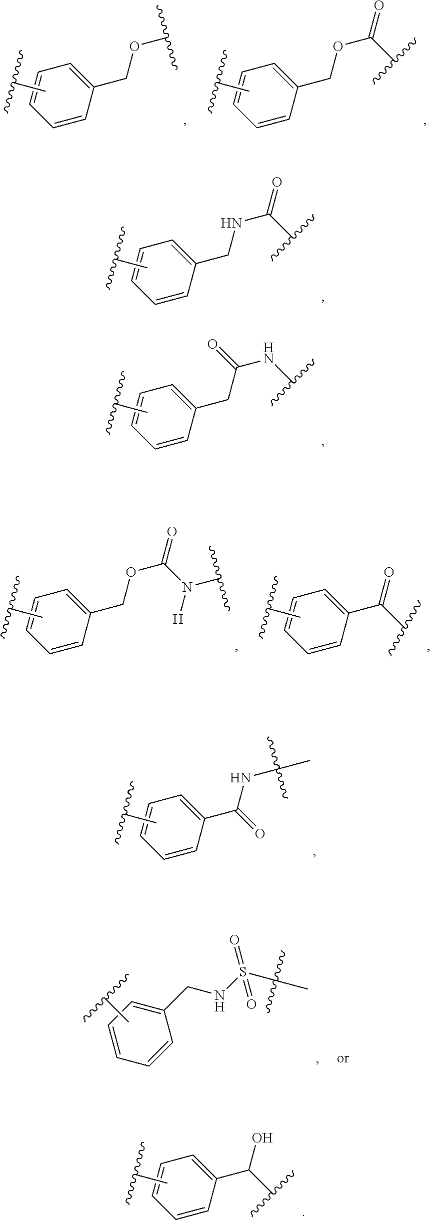

In some embodiments of Formula (IVd) or Formula (IVe), L^{C} is selected from the group consisting of

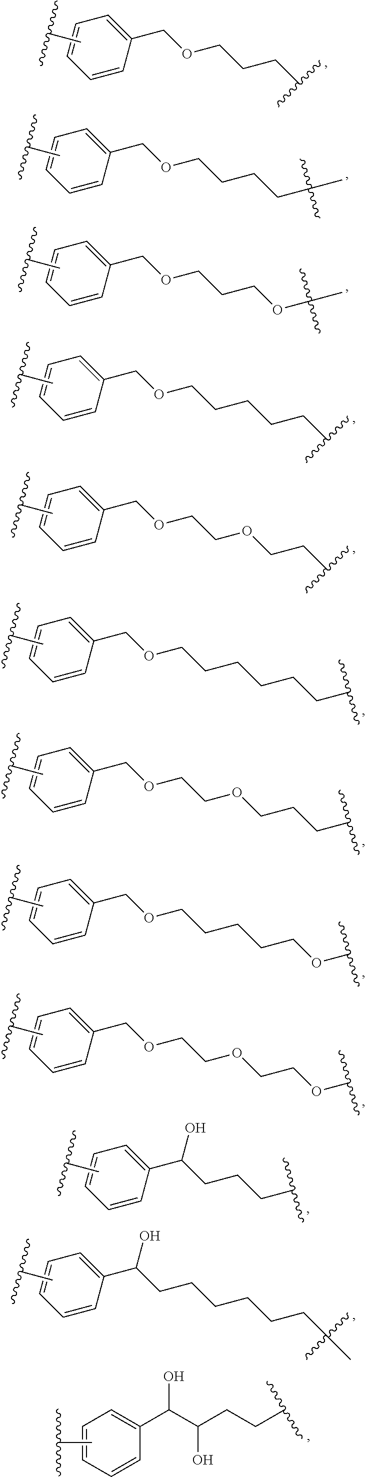

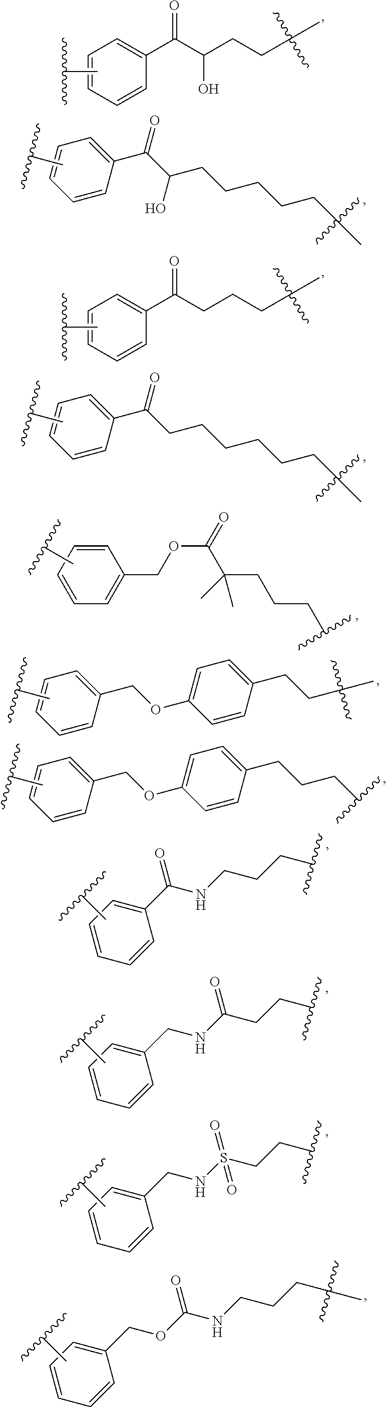

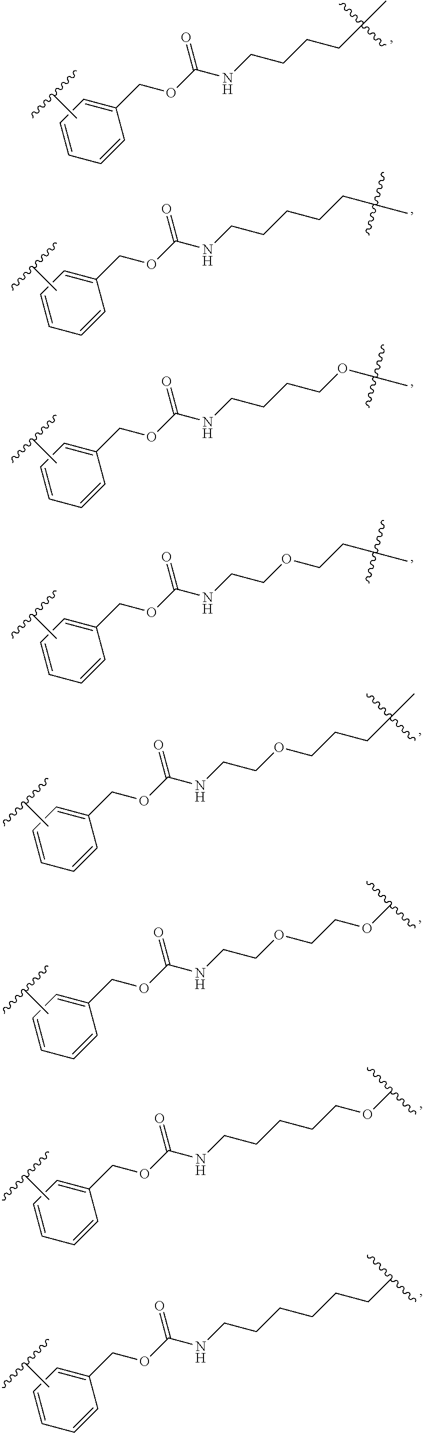

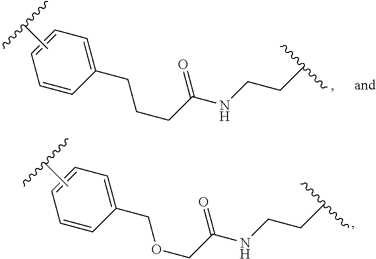

wherein R* is connected to the phenylene.

In some embodiments, the radiolabeled compound comprises a structure of Formula (IVd) or Formula (IVe), wherein L^{C}-R* is

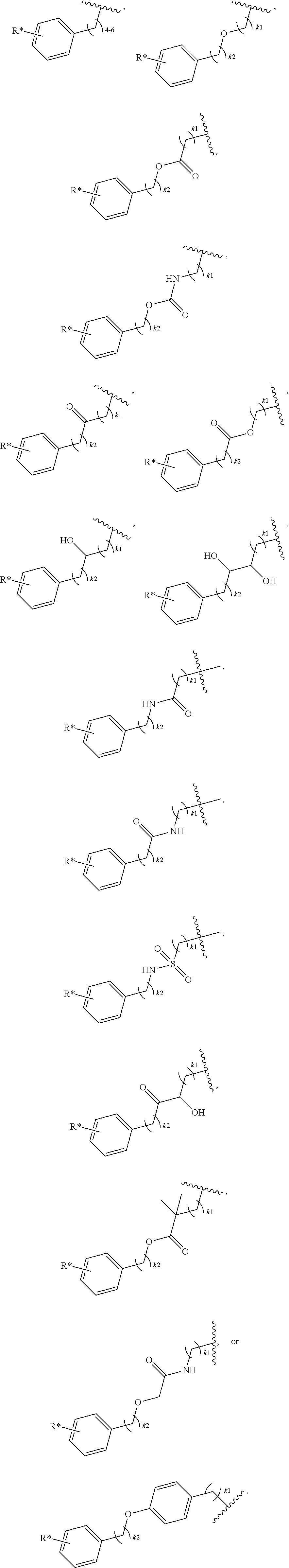

wherein each k1 and k2 is independently 0 or an integer selected from 1 to 10. In some embodiments, each k1 and k2 is independently 0 or an integer selected from 1 to 5. In some embodiments, k1 is 0 to 5 and k2 is 0 to 2. In some embodiments, k1 is 2 to 4 and k2 is 0 to 1.

In some embodiments, the radiolabeled compound comprises a structure of Formula (IVd) or Formula (IVe) wherein L^{C}-R* is

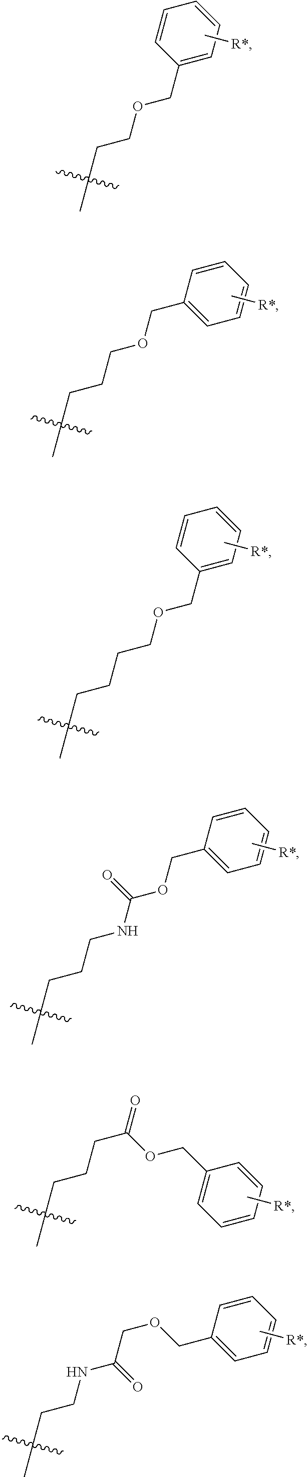

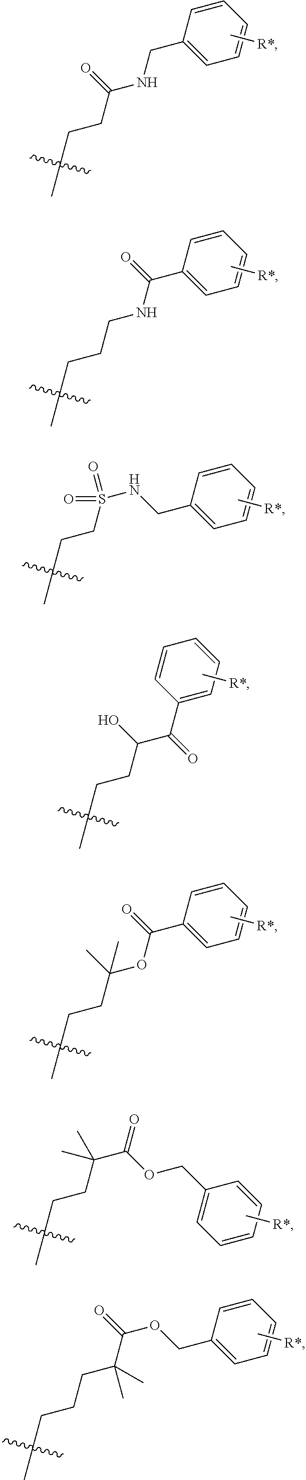

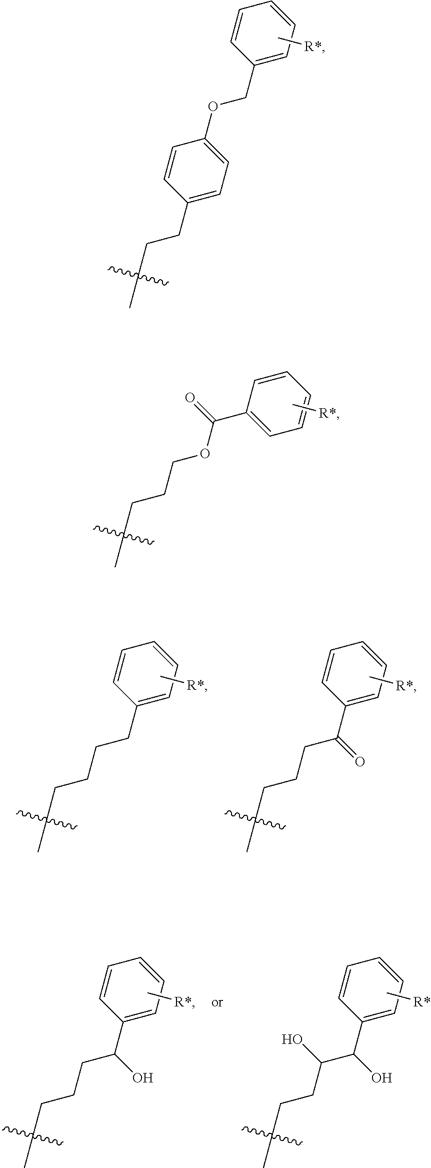

In some embodiments, R* is selected from a radioisotope in Table 6C or Table 6D. In some embodiments, R* is iodine-131 (¹³¹I) or astatine-211 (²¹¹At). In some embodiments, R* is iodine-131 (¹³¹I).

In some embodiments, provided herein are compounds having the structures of the radiolabeled compounds described herein (e.g., a compound of Formula (IV), Formula (IVa), Formula (IVb), Formula (IVc), Formula (IVd), Formula (IVe), or Formula (IVe), except that the radioisotope is replaced with a surrogate (e.g., ¹³¹I replaced with iodine), i.e., a cold compound. In some embodiments, a radionuclide of the radiolabeled compounds described herein can be replaced with a surrogate (e.g., ¹³¹I replaced with iodine) for testing and experimental purposes.

### Linkers

Provided herein are radiolabeled compounds that comprise a covalently bonded radioisotope. Provided herein are modified KRAS G12C proteins comprising a covalently bonded radiolabeled compound which further comprises a covalently bonded radioisotope. In some embodiments, the covalently bonded radioisotope is attached to the radiolabeled compound through a chemical linker. In some embodiments, the chemical linker is L^{C} as described herein. In some embodiments, the covalently bonded radioisotope can comprise one or more linkers. The one or more linkers can each independently binds a radioisotope. In some embodiments, the radioisotope is selected from a radioisotope in Table 6C or Table 6D. In some embodiments, the radioisotope is selected from fluorine-18 (¹⁸F), iodine-131 (¹³¹I), iosine-123 (¹²³I), iodine-124 (¹²⁹I), iodine-125 (¹²⁵I), or astatine-211 (²¹¹At). In some embodiments, the radioisotope is ¹³¹I. In some embodiments, the radioisotope is ¹²⁴I. In some embodiments, the radioisotope is ¹²⁵I. In some embodiments, the radioisotope is ²¹¹ At.

In some embodiments, the radioisotope is covalently bound to the linker as illustrated by a structure selected from Formula (Va), Formula (Vb), Formula (Vc), Formula (Vd), and Formula (Ve):

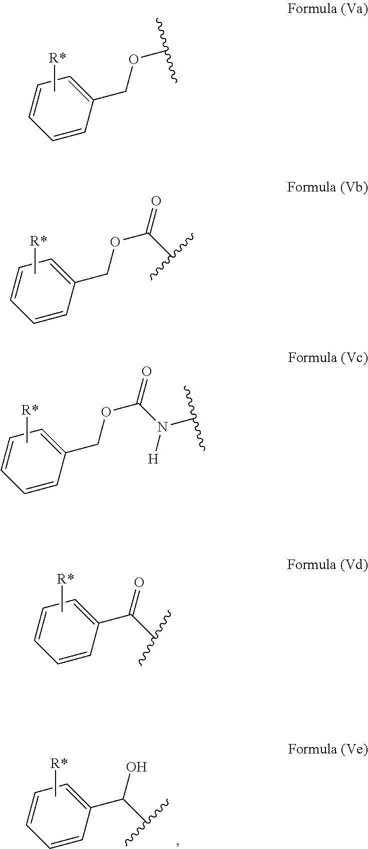

wherein,
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At); and
R^{a} is hydrogen, halogen, C₁-C₆alkyl, C₁-C₆haloalkyl, C₁-C₆hydroxyalkyl, C₁-C₆aminoalkyl, C₁-C₆heteroalkyl, C₁-C₆alkenyl, C₂-C₆alkynyl, C₃-C₈cycloalkyl, C₂-C₉heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl, or heteroaryl is optionally substituted.

For the avoidance of doubt, Formula (Va), Formula (Vb), Formula (Vc), Formula (Vd), and Formula (Ve) comprise all or a pan of a linker and the radioisotope R.

In some embodiments, of Formula (Va), Formula (Vb), Formula (Vc), Formula (Vd), or Formula (Ve) the alkyl, haloalkyl, hydroxyalkyl, aminoalkyl, heteroalkyl, alkenyl, alkynyl, cycloalkyl, heterocycloalkyl, aryl, or heteroaryl are independently optionally substituted by one or more halogen, amino, —OH, —NO₂, oxo, —CN, C₁₋₃ alkoxyl, C₁₋₃ alkyl and C₁₋₃ haloalkyl. In some embodiments, R^{a} is hydrogen. In some embodiments, R^{a} is C₁-C₄alkyl. In some embodiments, R^{a} is C₁-C₄cycloalkyl. In some embodiments. R^{a} is ¹³¹I.

In some embodiments, the radioisotope is covalently bound to the linker as illustrated by the following structures selected from Formula (Va), Formula (Vb), Formula (Vc), Formula (Vd), and Formula (Ve):

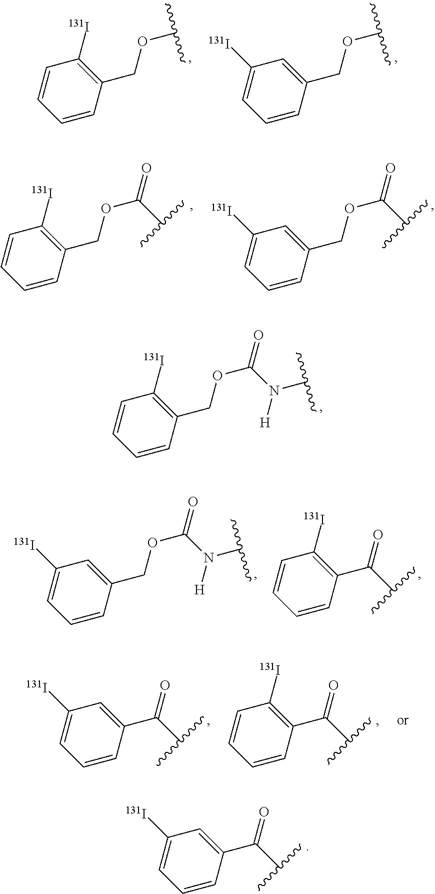

In some embodiments, the radiolabeled compound comprises a structure of Formula (Va), Formula (Vb), Formula (Vc), Formula (Vd), or Formula (Ve).

A linker described herein (such as group L^{C} of Formula (IIIb), (IIIc), (IVd) and (IVe)) can have a prescribed length thereby linking the radioisotope and the radiolabeled compound while allowing an appropriate distance therebetween. In some embodiments, the linker has 1 to 100 atoms, 1 to 60 atoms, 1 to 30 atoms, 1 to 15 atoms, 1 to 10 atoms, 1 to 5, or 2 to 20 atoms in length. In some embodiments, the linker has 1 to 10 atoms in length. In some embodiments, the linker has 1 to 10 atoms in length. In some embodiments, the linker is between 5 and 20 carbon atoms long. In some embodiments, the linker is between 2 and 18 carbon atoms long. In some embodiments, the linker is between 2 and 20 carbon atoms long. In some embodiments, the linker is between 5 and 10 atoms long. In some embodiments, the linker is between 10 and 15 atoms long. In some embodiments, the linker is between 15 and 20 atoms long. In some embodiments, the linker is between 10 and 20 atoms long.

A linker described herein can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences (e.g., EAAAK (SEQ ID NO: 3)), proline-rich sequences, spirocycles, hetercycloalkylene moieties, cycloalkylene moieties, and regions rich in double and/or triple bonds.

A linker described herein (such as group of Formula (IIIb). (IIIc), (IVd) and (IVe)) can be cleavable, e.g., under physiological conditions, e.g., under intracellular conditions. e.g., under extracellular conditions such that cleavage of the linker separates the radiolabeled compound from the covalently bonded radioisotope. In some embodiments, the linker is a peptidase-cleavable linker. The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin. In other embodiments, the linker is not cleavable. In some embodiments, the linker is pH-sensitive. i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable at below pH 5.5 or 5.0, the approximate pH of the lysosome. In some embodiments, the hydrolyzable linker is a thioether linker. In some embodiments, the linker is an esterase-cleavable linker. The linker can be, e.g., an ester containing linker that is cleaved by an esterase. In some embodiments, the linker can be cleaved in vivo by esterases present in the kidney, liver, plasma, or other tissue. In some embodiments, the linker is cleavable by carboxylesterase-1. In some embodiments, the linker is cleavable by carboxylesterase-2. In some embodiments, the linker is cleavable by butyrylcholinesterase (BChE). In some embodiments, the linker is cleavable by acetylcholinesterase (ACNE). In some embodiments, the linker is cleavable by paraoxonase (PON1). In some embodiments, the linker is cleavable by brush-border enzymes. In some embodiments, the linker comprises a brush-border enzyme cleavable sequence, e.g., glycine-tyrosine, glycine-O-methyltyrosine, glycine-lysine, Glycine-phenylalanine-lysine, methionine-valine, methionine-valine-lysine, glycine-aspartate, or glycine-glutamate. In some embodiments, the brush-border cleavable linker comprises glycine-lysine, glycine-tyrosine, glycine-phenylalanine-lysine, or methionine-valine-lysine. In some embodiments, the brush-border cleavable linker comprises glycine-lysine. In some embodiments, the brush-border cleavable linker comprises glycine-tyrosine. In some embodiments, the brush-border cleavable linker comprises glycine-phenylalanine-lysine. In some embodiments, the brush-border cleavable linker comprises methionine-valine-lysine. In some embodiments, the linker is a hepatocyte-cleavable linker. In some embodiments, the linker is metabolized by cytochrome P450. In some embodiments, the linker is cleaved by cytochrome P450. In some embodiments, the linker is metabolized or cleaved by cytochrome P450 3A4. In some embodiments, the linker is a cytochrome P450 substrate. In some embodiments, the linker is oxidized by cytochrome P450 3A4 and subsequently cleaved. In some embodiments, the linker is oxidized by flavin monooxygenase, monoamine oxidase, alcohol dehydrogenase, aldehyde dehydrogenase, aldehyde oxidase or xanthine oxidase.

In some embodiments, A linker described herein comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, the linker comprises polyethylene glycol such as (—CH₂—CH₂—O—)₁₋₁₀.

In some embodiments, A linker described herein (such as L^{C}) comprises a structure selected from Table 3A.

TABLE 3A

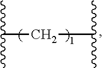

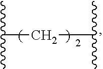

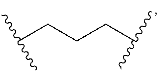

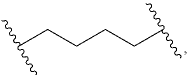

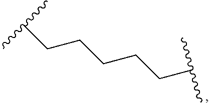

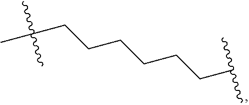

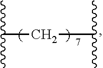

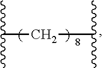

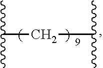

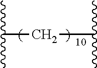

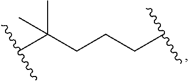

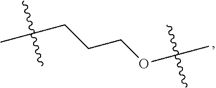

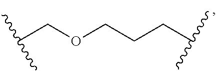

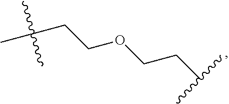

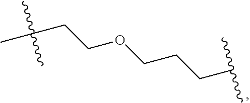

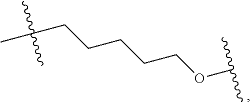

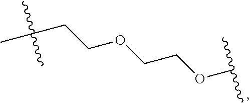

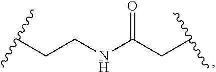

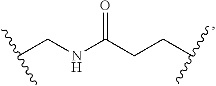

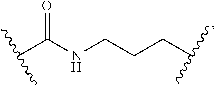

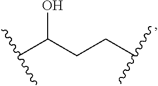

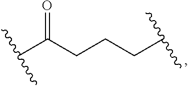

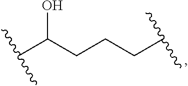

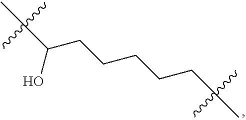

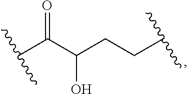

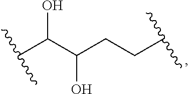

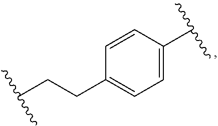

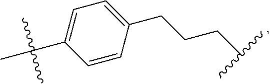

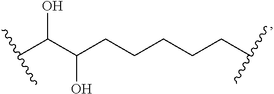

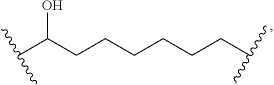

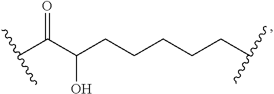

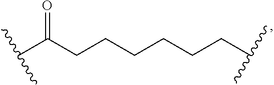

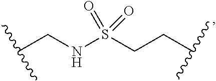

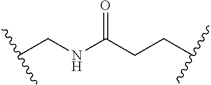

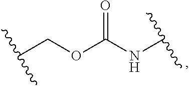

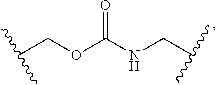

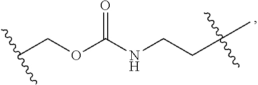

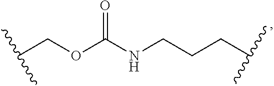

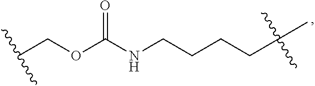

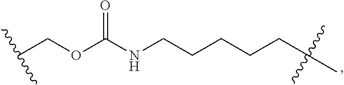

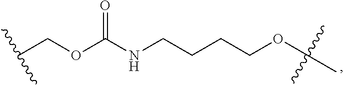

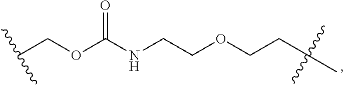

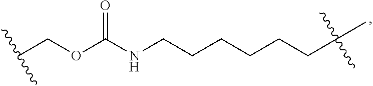

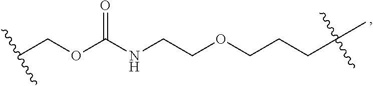

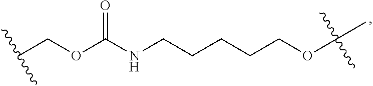

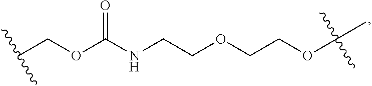

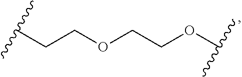

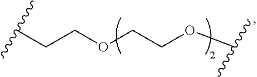

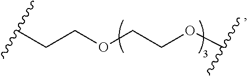

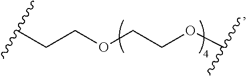

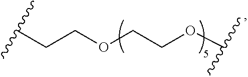

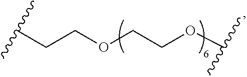

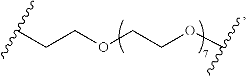

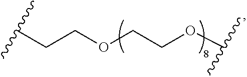

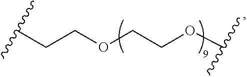

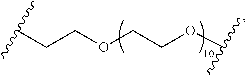

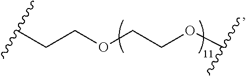

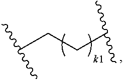

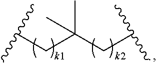

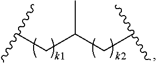

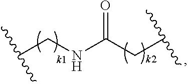

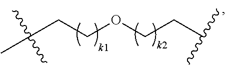

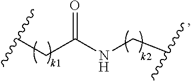

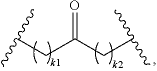

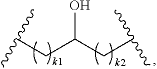

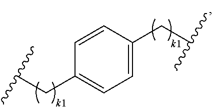

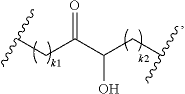

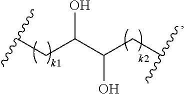

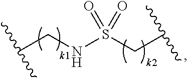

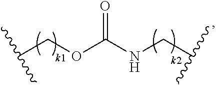

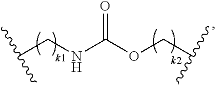

wherein each k1 and k2 is independently 0 or an integer selected from 1 to 20.

In some embodiments of Table 3A and Table 3C. k1 is selected from 0-12. In some embodiments, k1 is 0. In some embodiments, k1 is 1. In some embodiments, k1 is 2. In some embodiments, k1 is 3. In some embodiments, k1 is 4. In some embodiments, k1 is 5. In some embodiments, k1 is 6. In some embodiments, k1 is 7. In some embodiments, k1 is 8. In some embodiments, k1 is 9. In some embodiments, k1 is 10. In some embodiments of Table 3A and Table 3C. k2 is selected from 0-12. In some embodiments, k2 is 0. In some embodiments, k2 is 1. In some embodiments, k2 is 2. In some embodiments, k2 is 3. In some embodiments, k2 is 4. In some embodiments, k2 is 5. In some embodiments, k2 is 6. In some embodiments, k2 is 7. In some embodiments, k2 is 8. In some embodiments, k2 is 9. In some embodiments, k2 is 10.

In some embodiments, a linker described herein comprises a structure selected from Table 3B.

TABLE 3B

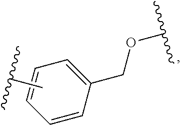

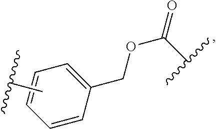

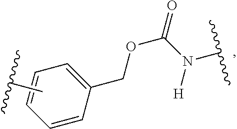

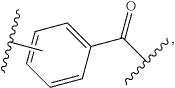

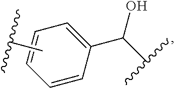

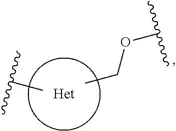

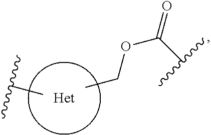

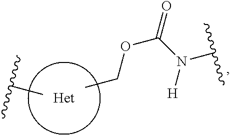

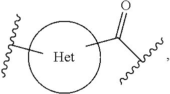

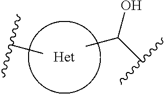

wherein Het is a 5-6 membered heteroaryl ring containing 1-3 heteroatoms independently selected from N. S, and O. In some embodiments, Het is pyridinyl or pyrimidinyl.

In some embodiments, a linker described herein comprises a structure selected from Table 3C.

TABLE 3C

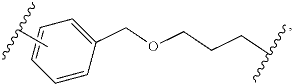

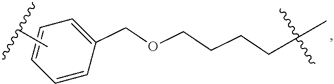

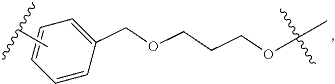

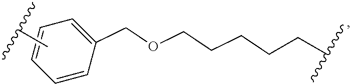

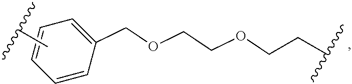

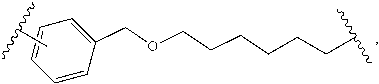

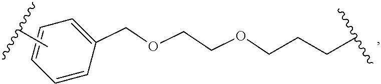

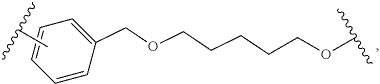

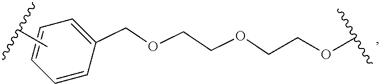

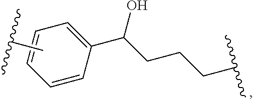

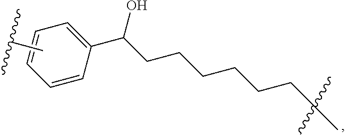

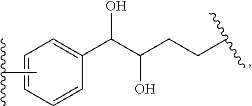

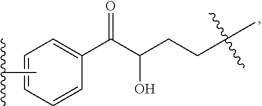

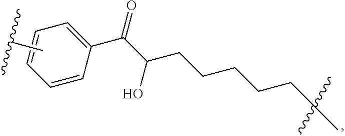

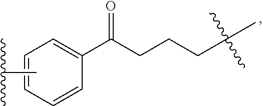

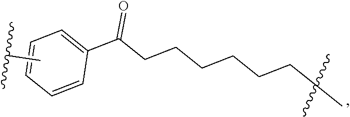

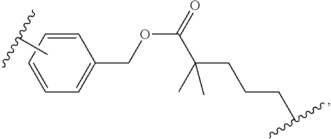

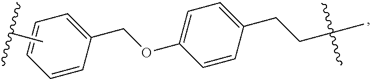

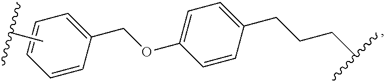

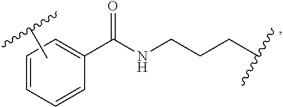

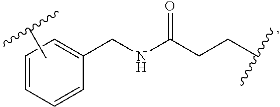

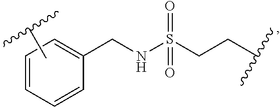

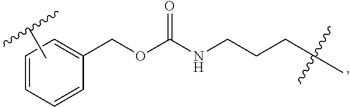

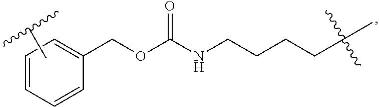

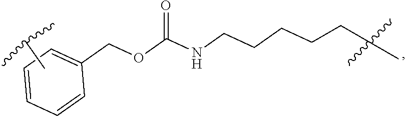

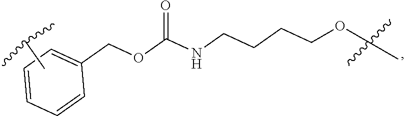

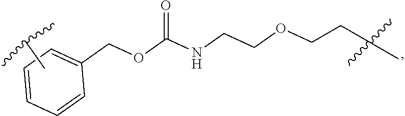

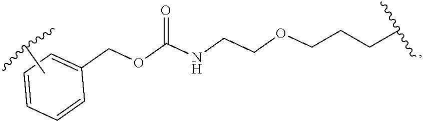

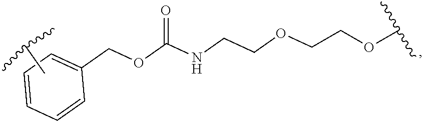

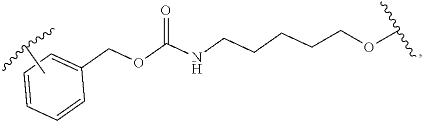

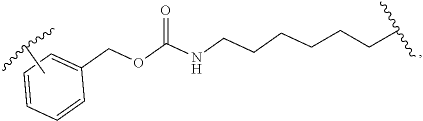

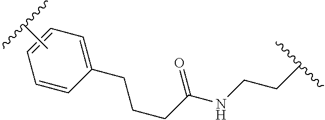

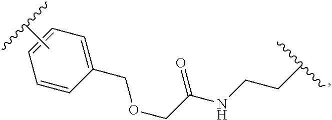

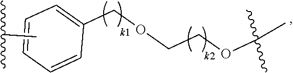

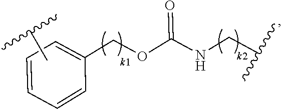

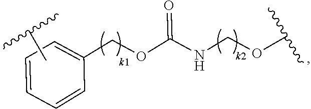

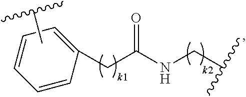

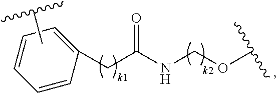

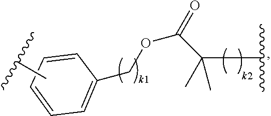

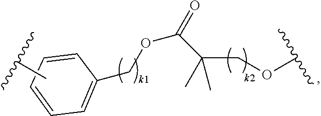

and k2 is independently 0 or an integer selected from 1 to 10.

In some embodiments, a linker described herein (e.g., L^{C}) comprises a structure in Table 3A. Table 3B, or Table 3C. In some embodiments, a linker described herein (e.g., L^{C}) comprises a structure in Table 2A in combination with a structure in Table 3B. In some embodiments, a linker described herein (e.g., L^{C}) consists of a structure in Table 2A in combination with a structure in Table 3B. In some embodiments, a linker described herein (e.g., L) consists of a structure in Table 3C. In some embodiments, the radionuclide is attached to the phenylene or heteroarylene moiety in Table 3B or Table 3C. In some embodiments, a -LK¹-LK²-LK³ linker described herein (e.g., in Formula (X-III) and Formula (X-IV)) comprises a structure in Table 3A, Table 3B, or Table 3C. In some embodiments, a -LK¹-LK²-LK³ linker described herein (e.g., in Formula (X-III) and Formula (X-IV)) comprises a structure in Table 2A in combination with a structure in Table 3B. In some embodiments, a -LK¹-LK²-LK³ linker described herein (e.g., in Formula (X-III) and Formula (X-IV)) consists of a structure in Table 2A in combination with a structure in Table 3B. In some embodiments, a -LK¹-LK²-LK³ linker described herein (e.g., in Formula (X-III) and Formula (X-IV)) consists of a structure in Table 3C.

In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, the linker comprises polyethylene glycol such as (—CH₂—CH₂—O—)₁₋₁₀. In some embodiments, the linker comprises a structure selected from:

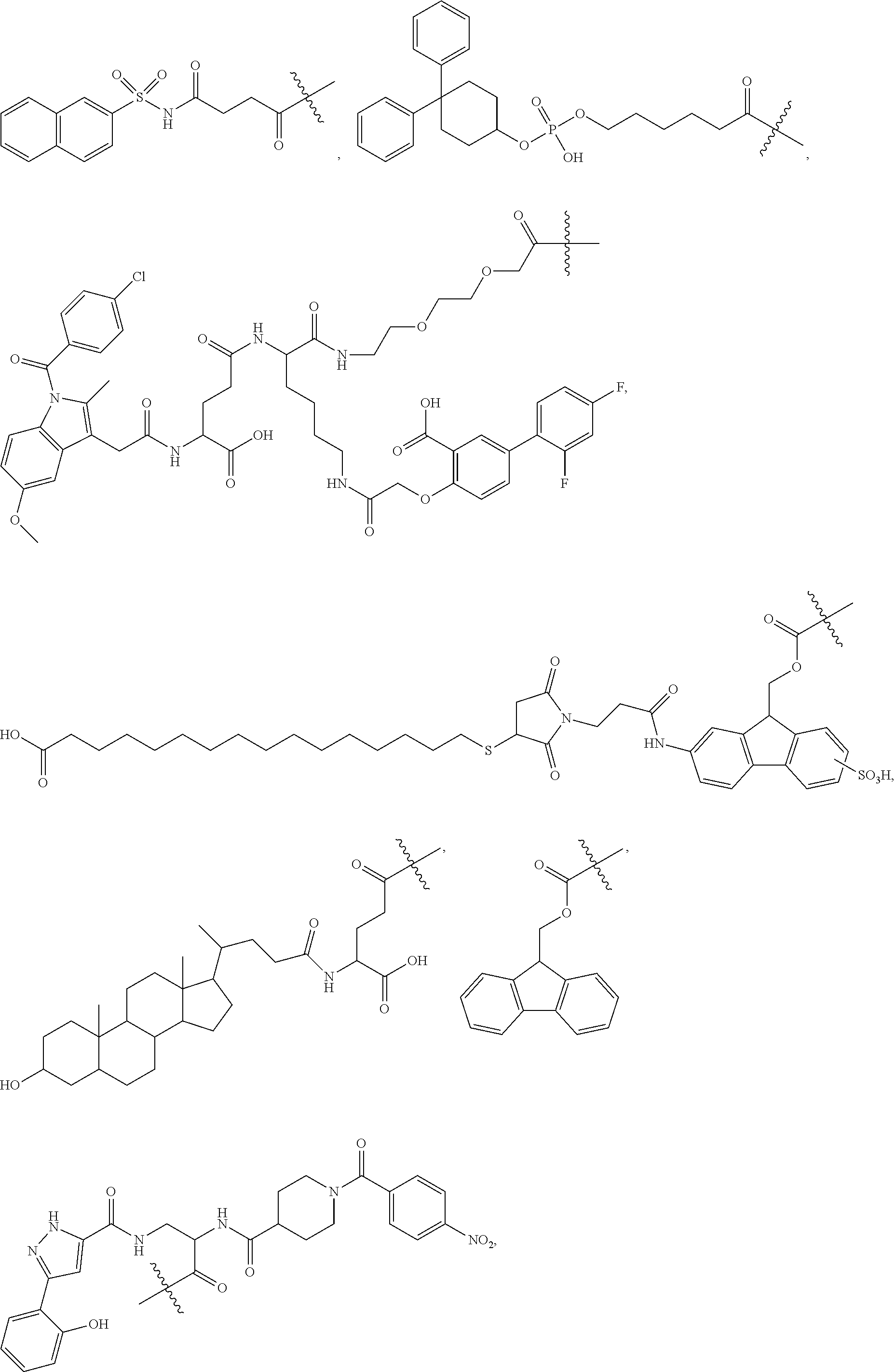

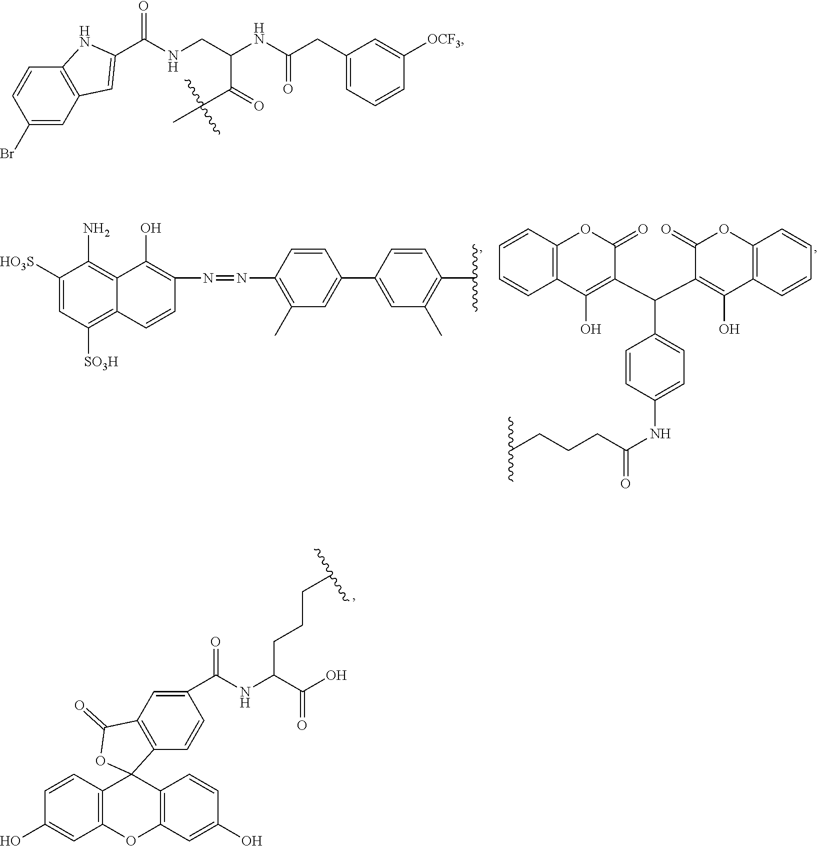

and structures derived from any one thereof.

In some embodiments, the linker comprises a click chemistry residue. In some embodiments, the linker is attached to the peptide, to the metal chelator, or both via click chemistry, thereby forming a click chemistry residue. For example, the peptide can comprise an azide group (at N- or C-terminus or at a non-terminal amino acid) that reacts with an alkyne moiety of the linker. For another example, the peptide can comprise an alkyne group (at N- or C-terminus or at a non-terminal amino acid) that reacts with an azide of the linker. The metal chelator and the linker can be attached similarly. In some embodiments, the linker comprises an azide moiety, an alkyne moiety, or both. In some embodiments, the linker comprises a triazole. In some embodiments, the click chemistry residue is

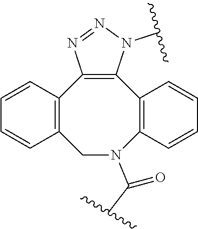 [SMILES: *C(=O)N1Cc2ccccc2-c2nnn(*)c2-c2ccccc21]

(DBCO-azide residue),

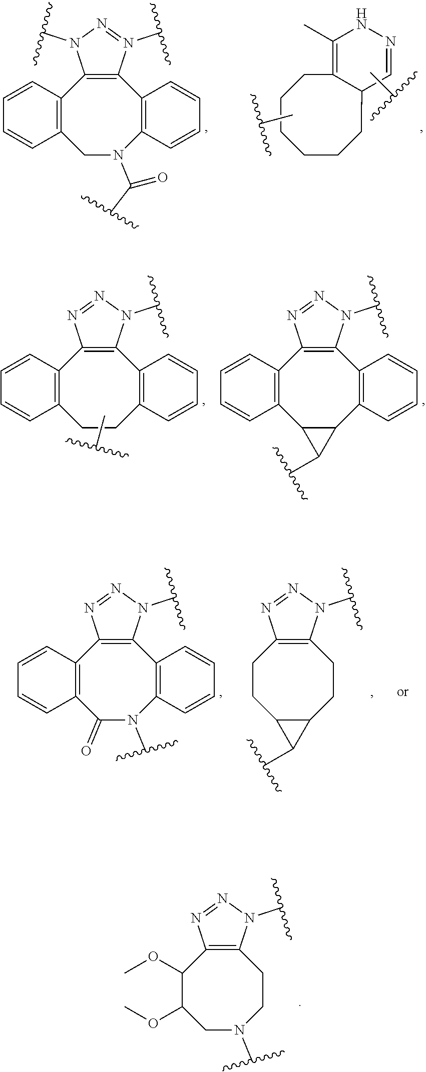

In some embodiments, the click chemistry residue is a DIBO-azide residue. BARAC-azide residue, DBCO-azide residue, DIFO-azide residue, COMBO-azide residue, BCN-azide residue, or DIMAC-azide residue. In some embodiments, the linker comprises a residue of nitrone dipole cycloaddition. In some embodiments, the linker comprises a residue of tetrazine ligation. In some embodiments, the linker comprises a residue of quadricyclane ligation. Exemplary groups of click chemistry residue are shown in Hein at al., “Click Chemistry. A Powerful Tool for Pharmaceutical Sciences,” Pharmaceutical Research volume 25, pages 2216-2230 (2008); Thirumurugan et al, “Click Chemistry for Drug Development and Diverse Chemical-Biology Applications.” Chem. Rev. 2013, 113, 7, 4905-4979: US20160107999A1; U.S. Ser. No. 10/266,502B2; and US20190204330A1, each of which is incorporated by reference in its entirety.

In some embodiments, a linker of the present disclosure (e.g., L^{C} and -LK¹-LK²-LK³) comprises at least one group selected from the group consisting of a bond, alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene, is optionally substituted. In some embodiments, the alkylene, alkenylene, alkynylene, cycloalkylene, arylene, heteroalkylene, heterocycloalkylene or heteroarylene are each independently substituted with one or more groups, each substituent group being independently selected from the group consisting of —O—, —S—, silicone, amino, optionally substituted alkyl (e.g., alkoxy, haloalkyl) and optionally substituted heterocycloalkylene (e.g., polyTHF). In some embodiments, the linker comprises substituted or unsubstituted C₁-C₁₀ alkylene or substituted or unsubstituted C₁-C₁₀ heteroalkylene. In some embodiments, the C₁-C₁₀ alkylene or C₁-C₁₀ heteroalkylene is substituted with one or more substituents selected from halogen, amino, —OH, —NO₂, oxo, —CN, C₁₋₃ alkoxyl, C₁₋₃ alkyl, C₁₋₃ hydroxyalkyl, C₁₋₃ aminoalkyl, and C₁₋₃ haloalkyl. In some embodiments, the linker comprises substituted or unsubstituted C₁-C₆ alkylene or substituted or unsubstituted C₁-C₁₀ heteroalkylene. In some embodiments, the C₁-C₆ alkylene or C₁-C₆ heteroalkylene is substituted with one or more substituents selected from halogen, amino. —OH, —NO₂, oxo, —CN, C₁₋₃ alkoxyl, C₁₋₃ alkyl, C₁₋₃ hydroxyalkyl, C₁₋₃ aminoalkyl, and C₁₋₃ haloalkyl. In some embodiments, the linker is or comprises propyl ethyl ether.

In some embodiments, the linker is or comprises at least one amino acid. In some embodiments, the linker L is or comprises two amino acids. In some embodiments, the linker L is or comprises three amino acids.

In some embodiments, a linker of the present disclosure comprises one or more groups selected from —O—, —S—, —S—S—, —NH—, —NH—(CH₂), —NH, —NH—(CH₂)p-O, —O—(CH₂)ₚ—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)_{q}, (C═O)—, —(C═O)—(CH═CH)_{q}—(C═O), —(C═O)—(OCH₂CH₂O)_{q}(C═O)—, —(CH₂CH═O)_{q}, —(OCH₂CH₂)_{q}, —(C═O)—(CH₂CH₂O)_{q}— and —(CH(CH₃)C(═O)O)_{q}— wherein q is 1-20 and p is 1-20. In some embodiments, the linker is or comprises a polyethylene glycol (PEG) or polypropylene glycol (PPG) linker. In some embodiments, the linker is or comprises —(CH₂CH₂O)_{q} or —(OCH₂CH₂)_{q}—. In some embodiments, the linker comprises —O—. In some embodiments, the linker comprises substituted or unsubstituted C₁-C₆ alkylene. In some embodiments, q is 1, 2, 3, 4, 5, 6, 7, 8.9, or 10. In some embodiments, p is 1, 2, 3.4, 5, 6, 7, 8, 9, or 10. In some embodiments, a linker of the present disclosure comprises —C(O)NH— or —NHC(═O)—. In some embodiments, a linker of the present disclosure comprises —NHC(═O)—O— or —OC(═O)—NH—.

In some embodiments, a linker of the present disclosure comprises 1 to 20 groups independently selected from —CR^{a}R^{b}—, —C(═O), —S(═O)—, —S(═O)₂—, —NR^{a}—,

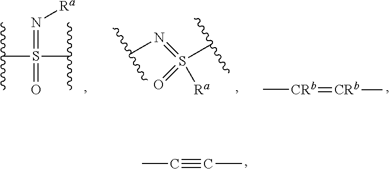

—O—, —S—, —C(═O)O—, —OC═O—C O NR^{a}—, —NR^{a}C, —S(═O)₂NR^{a}—, —NR^{a}S(═O)₂—, —NR^{a}C(═O)NR^{a}—, —NR^{a}C(═O)O—, —OC(═O)NR^{a}—, arylene, heteroarylene,
wherein each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₁-C₆alkynyl, C₃-C₈cycloalkyl, C₂-C₆heterocycloalkyl, aryl, or heteroaryl, and
wherein each R^{b} is independently hydrogen, halogen, —CN, —NO₂, —OR^{a}, —SR^{a}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl.

In some embodiments, a linker of the present disclosure comprises 1 to 5, 1 to 3, or 1 to 10 groups as described above.

In some embodiments, a linker of the present disclosure (e.g., L^{C} and -LK¹-LK²-LK³) comprises

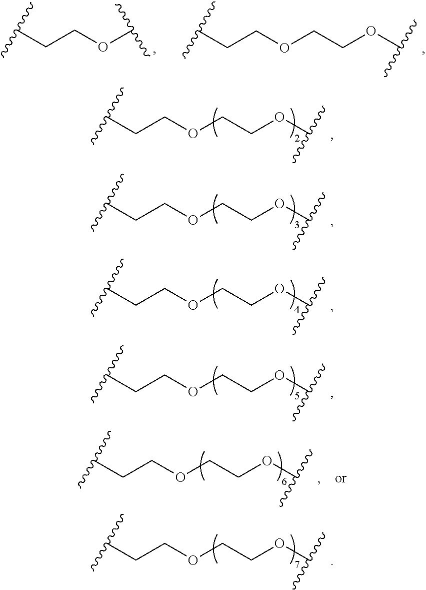

In some embodiments, a linker of the present disclosure comprises

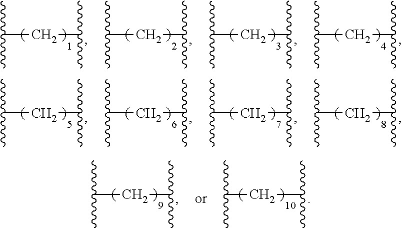

In some embodiments, the linker has a structure of

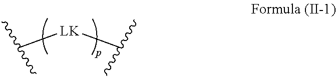

wherein each LK is independently —O—, —R^{LK}—, —N(R^{LK})₂⁺—, —OP(═O)(OR)O—, —S—, —S(═O)—, —S(═O)₂—, ═CH—, —C(═O)—, —C(═O)O—, —OC(═O)—, —OC(═O)O—, —C(═O)NR^{LK}—, —NR^{LK}C(═O)—, —OC(═O)NR^{LK}, —NR^{LK}C(═O)O—, —NR^{LK}C(═S)NR^{LK}—, —CR^{LK}═N—, —N═CR^{LK}, —NR^{LK}S(═O)₂—, —S(═O)₂NR^{LK}—, —C(═O)NR^{LK}S(═O)₂—, —S(═O)₂NR^{LK}—, C(═O)—, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, substituted or unsubstituted cycloalkylene, substituted or unsubstituted heterocycloalkylene, —(C₁-C₃₀ alkylene)-O—, —O—(C₁-C₃₀ alkylene)-, —(C₁-C₃₀ alkylene)—NR^{LK}—, —NR^{LK}—(C₁-C₃₀ alkylene)-, —(C₁-C₃₀ alkylene)-N(R^{LK})₂—, —N(R^{LK})—(C₁-C₃₀ alkylene)-, or a click chemistry residue; and
each R^{LK} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
p is 1, 2, 3, 4, 5, 6, 7, 8, 9, 10, 11, 12, 13, 14, or 15.

In some embodiments, the linker has a structure of

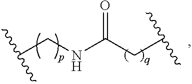

wherein each of q and p is independently 0, 1, 2, 3, 4, 5, 6, 7, 8, 9, 10, 11, 12, 13, 14, or 15. In some embodiments, the linker has a structure of

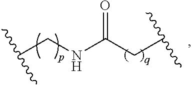

wherein each of q and p is independently 0, 1, 2, 3, 4, 5, 6, 7, 8, 9, 10, 11, 12, 13, 14, or 15, and each of methylene can be substituted or unsubstituted. In some embodiments, p is 0, 1, 2, 3,4, or 5. In some embodiments, q is 0, 1, 2.3, 4, or 5.

In some embodiments, the linker has a structure of

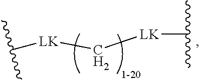

wherein each LK is independently —O—, —NR^{LK}—, —N(R^{LK})₂—, —OP(O)(OR)O—, —S—, —S(O)—, —S(O)₂—, —CH═CH—, ═CH—, —C═C—, —C(═O)—, —C(═O)—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}S(═O)₂—, —S(═O)₂NR^{LK}—, —C(═O)NR^{LK}S(═O)₂—, or —S(═O)₂NR^{LK}C(═O)—.

In some embodiments, the linker comprises substituted or unsubstituted C₁-C₃₀ alkylene, C₁-C₁₂ alkylene, C₁-C₈ alkylene, C₁-C₆ alkylene, or C₂-C₆ alkylene. In some embodiments, the linker comprises C₂-C₆ alkylene. In some embodiments, the linker comprises C₄-C₆ alkylene.

In some embodiments, the linker has a structure of

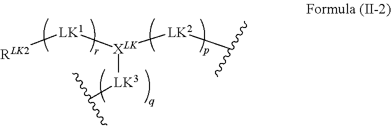

wherein each LK¹, LK², and LK³ is independently —O—. —NR^{LK}—, —N(R^{LK})₂⁺—, —OP(═O)(OR^{LK})O—, —S—, —S(═O)—, —S(═O)₂—, ═CH—, —C(═O)—, —C(═O)O—, —OC(═O)—, —OC(═O)O—, —C(═O)NR^{LK}—, —NR^{LK}C(═O)—, —OC(═O)NR—, —NR^{LK}C(═O)O—, —NR^{LK}C(═O)NR^{LK}—, —NR^{LK}C(═S)NR^{LK}—, —CR^{LK}═N—, —N═CR, —NR^{LK}S(═O)₂—, —S(═O)₂NR^{LK}—, —C(═O)NR^{LK}S(═O)₂—, —S(═O), NR^{LK}C(═O)—, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, substituted or unsubstituted C₁-C₁₅ arylene, —(C₁-C₆ alkylene)-O—, —O—(C₁-C₃₀ alkylene)-, —(C₁-C₁₅ alkylene)—NR^{LK}—, —NR^{LK}—(C₁-C₃₀ alkylene)-, —(C₁-C₃₀ alkylene)-N(R^{LK})₂⁺—, —N(R^{LK})₂—(C₁-C₃₀ alkylene)-, substituted or unsubstituted cycloalkylene, substituted or unsubstituted heterocycloalkylene, or a click chemistry residue, and
R^{LK2} is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₇ alkynyl, substituted or unsubstituted cycloalkynyl, substituted or unsubstituted C₃-C₃₀ cycloalkyl, substituted or unsubstituted C₂-C₆heterocycloalkyl, substituted or unsubstituted aryl, or substituted or unsubstituted heteroaryl;
R^{LK} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
X^{LK} is N or CR^{LK}; and
   each of r, p, and q is independently 0, 1, 2, 3, 4, 5, 6, 7, 8, 9, 10, 11, 12, 13, 14, or 15.

In some embodiments, the linker has a structure of

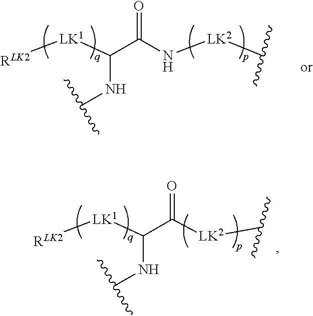

wherein
wherein each LK¹ and LK² is independently —O—, —NR^{LK}—, —N(R^{LK})₂⁺—, —OP(O)(OR^{LK})O—, —S—, —S(═O)—, —S(═O)₂—, —CH═CH—. ═CH—, —C═C—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—. —NR^{LK}C(═O)NR^{LK}—, —NR^{LK}S(═O)₂—, —S(═O)₂NR^{LK}—, —C(═O)NR^{LK}(═O)₂—, —S(═O)₂NR^{LK}C(═O)—, substituted or unsubstituted cycloalkylene, substituted or unsubstituted heterocycloalkylene, substituted or unsubstituted C₁-C₂₀ alkylene, or —(CHR^{LK}—CHR^{LK}—O).
R^{LK} is hydrogen, substituted or unsubstituted C₁-C₆ alkyl, substituted or unsubstituted C₁-C₄ heteroalkyl, substituted or unsubstituted C₂-C₆ all enyl, substituted or unsubstituted C₂-C₅ alkynyl, substituted or unsubstituted C₃—C cycloalkyl, or substituted or unsubstituted C₂-C₇ heterocycloalkyl;
R^{LK2} is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₅ cycloalkyl, substituted or unsubstituted C₂-C₆ heterocycloalkyl, substituted or unsubstituted aryl, or substituted or unsubstituted heteroaryl;
q is 0-5, and p is 0-5.

In some embodiments, LK² is —O—. NR^{LK}—, —N(R^{LK})₂—, —OP(═O)(OR^{LK})O—, —S—, —S(O)—, —S(═O)₂—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, NR^{LK}S(═O)—, —S(═O)₂NR—, —C(O)NRS(O)₂, —S(═O)₂NR^{LK}C(═O)—, substituted or unsubstituted C₁-C₆ alkylene, or —(CH₂—CH₂—O)₁₋₆—.

In some embodiments, LK¹ is —O—, —NR^{LK}—, —N(R^{LK})₂—, —OP(O)(OR^{LK})O— —S—, —S(O)—, —S(═O)₂—, —CH═CH—, ═CH—, —C═C—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}S(═O)₂—, —S(═O)₂NR—, —C(═O)NR^{LK}(═O)₂—, —S(═O)₂NR^{LK}C(═O)—, substituted or unsubstituted C₁-C₂₀ alkylene, or —(CH₂—CH₂—O)₁₋₆₋.

In some embodiments, R^{LK} is hydrogen or substituted or unsubstituted C₁-C₄ alkyl.

In some embodiments, R^{LK2} is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl.

In some embodiments, p is 1, 2, 3, 4, or 5. In some embodiments, q is 1, 2, 3, 4, or 5.

In some embodiments, R^{LK2} is hydrogen, substituted or unsubstituted C₆-C₁₀ aryl, substituted or unsubstituted C₅-C₉ heteroaryl, or a sterol.

In some embodiments, at least one LK¹ is unsubstituted C₃-C₂₀ alkylene.

In some embodiments, the linker comprises one or more of a substituted or unsubstituted C₆-C₁₀ aryl, substituted or unsubstituted C₅-C₉ heteroaryl, a sterol, sulfonamide, phosphate ester, polyethylene glycol, or C₃-C₂₀ alkylene, or amino acid residues.

In some embodiments, the linker is configured to reversibly bind to a plasma protein such as albumin. In some embodiments, a dissociation constant (Kd) between the linker and human serum albumin is at most 15 μM, as determined at room temperature in human serum condition. In some embodiments, the Kd is from about 0.1 nM to about 10 μM. In some embodiments, the Kd is from about 10 nM to about 10 μM. In some embodiments, the Kd is from about 50 nM to about 1 μM. In some embodiments, the Kd is from about 1(a) nM to about 10 μM.

Exemplary configurations of the radiolabeled compound described herein are illustrated in Table 4A. Table 4B, Table 4C, and Table 4D.

TABLE 4A

| Compound | |
|---|---|
| ID | Structure |
| A-1 | 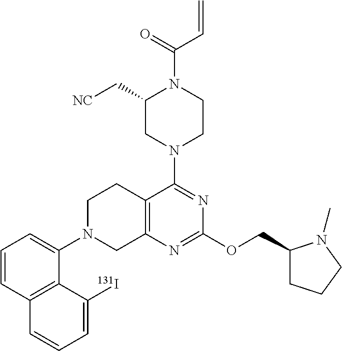 [SMILES: C=CC(=O)N1CCN(c2nc(OC[C@@H]3CCCN3C)nc3c2CCN(c2cccc4cccc([131I])c24)C3)C[C@@H]1CC#N] |
| A-2 | 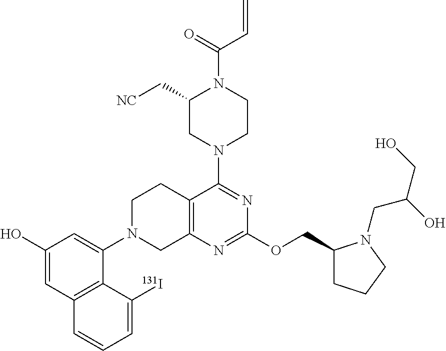 [SMILES: C=CC(=O)N1CCN(c2nc(OC[C@@H]3CCCN3CC(O)CO)nc3c2CCN(c2cc(O)cc4cccc([131I])c24)C3)C[C@@H]1CC#N] |
| A-3 | 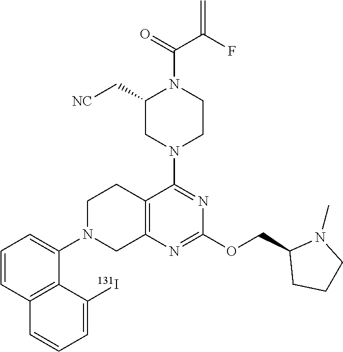 [SMILES: C=C(F)C(=O)N1CCN(c2nc(OC[C@@H]3CCCN3C)nc3c2CCN(c2cccc4cccc([131I])c24)C3)C[C@@H]1CC#N] |
| A-4 | 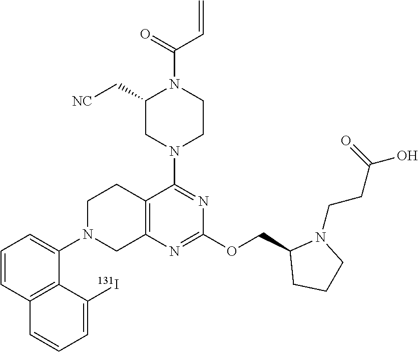 [SMILES: C=CC(=O)N1CCN(c2nc(OC[C@@H]3CCCN3CCC(=O)O)nc3c2CCN(c2cccc4cccc([131I])c24)C3)C[C@@H]1CC#N] |
| A-5 | 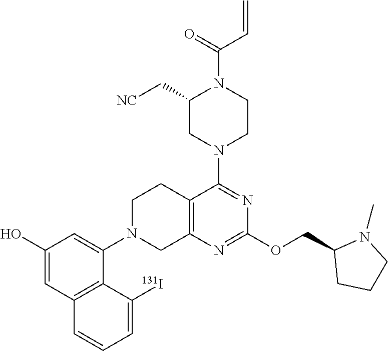 [SMILES: C=CC(=O)N1CCN(c2nc(OC[C@@H]3CCCN3C)nc3c2CCN(c2cc(O)cc4cccc([131I])c24)C3)C[C@@H]1CC#N] |
| A-6 | 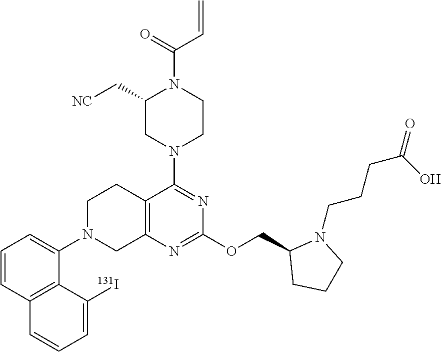 [SMILES: C=CC(=O)N1CCN(c2nc(OC[C@@H]3CCCN3CCCC(=O)O)nc3c2CCN(c2cccc4cccc([131I])c24)C3)C[C@@H]1CC#N] |
| A-7 | 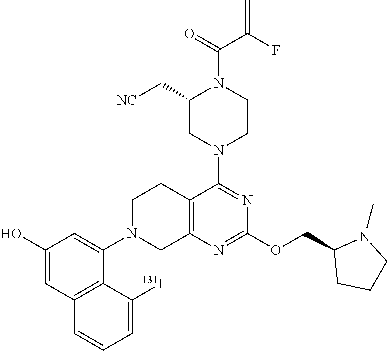 [SMILES: C=C(F)C(=O)N1CCN(c2nc(OC[C@@H]3CCCN3C)nc3c2CCN(c2cc(O)cc4cccc([131I])c24)C3)C[C@@H]1CC#N] |
| A-8 | 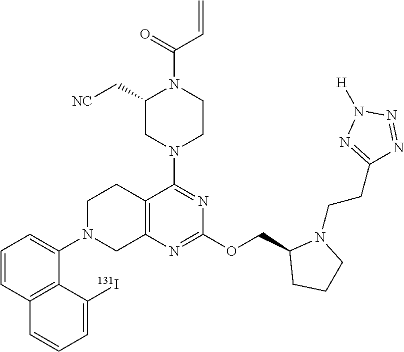 [SMILES: C=CC(=O)N1CCN(c2nc(OC[C@@H]3CCCN3CCc3nn[nH]n3)nc3c2CCN(c2cccc4cccc([131I])c24)C3)C[C@@H]1CC#N] |
| A-9 | 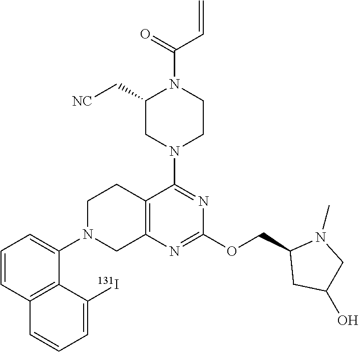 [SMILES: C=CC(=O)N1CCN(c2nc(OC[C@@H]3CC(O)CN3C)nc3c2CCN(c2cccc4cccc([131I])c24)C3)C[C@@H]1CC#N] |
| A-10 | 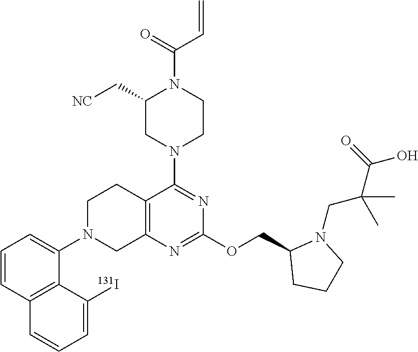 [SMILES: C=CC(=O)N1CCN(c2nc(OC[C@@H]3CCCN3CC(C)(C)C(=O)O)nc3c2CCN(c2cccc4cccc([131I])c24)C3)C[C@@H]1CC#N] |
| A-11 | 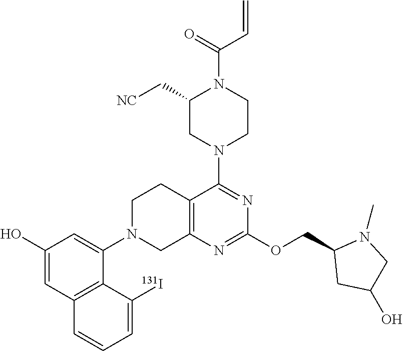 [SMILES: C=CC(=O)N1CCN(c2nc(OC[C@@H]3CC(O)CN3C)nc3c2CCN(c2cc(O)cc4cccc([131I])c24)C3)C[C@@H]1CC#N] |
| A-12 | 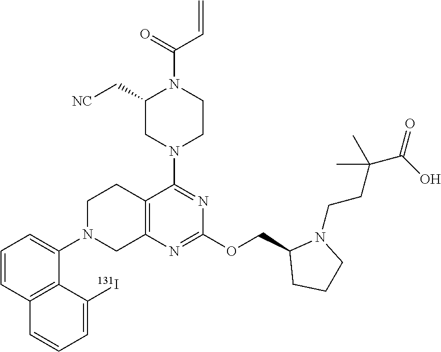 [SMILES: C=CC(=O)N1CCN(c2nc(OC[C@@H]3CCCN3CCC(C)(C)C(=O)O)nc3c2CCN(c2cccc4cccc([131I])c24)C3)C[C@@H]1CC#N] |
| A-13 | 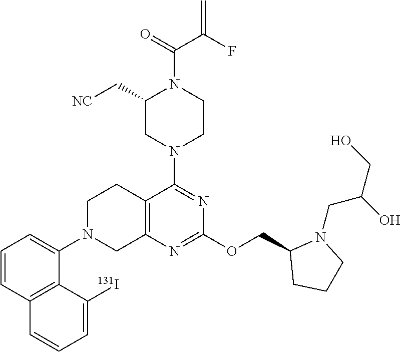 [SMILES: C=C(F)C(=O)N1CCN(c2nc(OC[C@@H]3CCCN3CC(O)CO)nc3c2CCN(c2cccc4cccc([131I])c24)C3)C[C@@H]1CC#N] |
| A-14 | 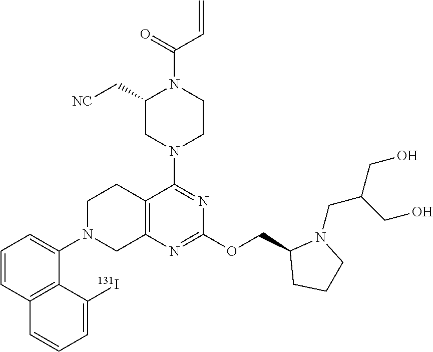 [SMILES: C=CC(=O)N1CCN(c2nc(OC[C@@H]3CCCN3CC(CO)CO)nc3c2CCN(c2cccc4cccc([131I])c24)C3)C[C@@H]1CC#N] |
| A-15 | 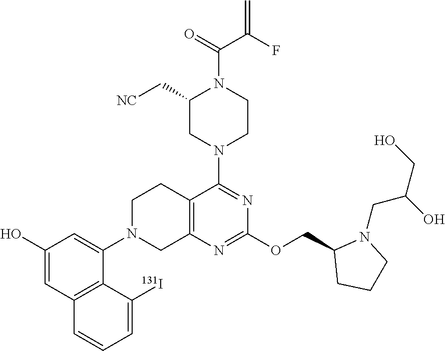 [SMILES: C=C(F)C(=O)N1CCN(c2nc(OC[C@@H]3CCCN3CC(O)CO)nc3c2CCN(c2cc(O)cc4cccc([131I])c24)C3)C[C@@H]1CC#N] |
| A-16 | 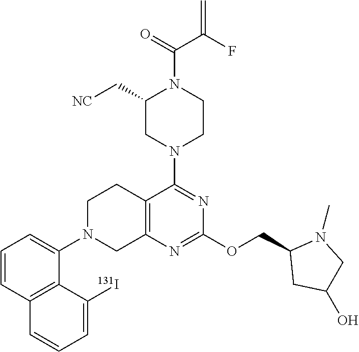 [SMILES: C=C(F)C(=O)N1CCN(c2nc(OC[C@@H]3CC(O)CN3C)nc3c2CCN(c2cccc4cccc([131I])c24)C3)C[C@@H]1CC#N] |
| A-17 | 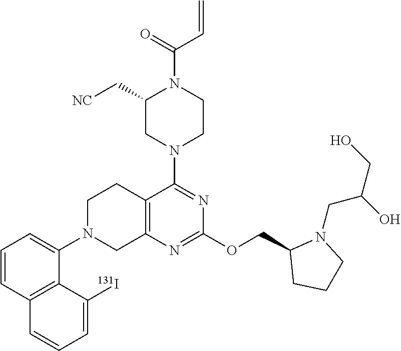 [SMILES: C=CC(=O)N1CCN(c2nc(OC[C@@H]3CCCN3CC(O)CO)nc3c2CCN(c2cccc4cccc([131I])c24)C3)C[C@@H]1CC#N] |
| A-18 | 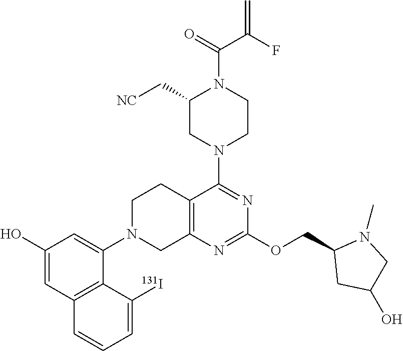 [SMILES: C=C(F)C(=O)N1CCN(c2nc(OC[C@@H]3CC(O)CN3C)nc3c2CCN(c2cc(O)cc4cccc([131I])c24)C3)C[C@@H]1CC#N] |

TABLE 4B

| Compound | |
|---|---|
| ID | Structure |
| B-1 | 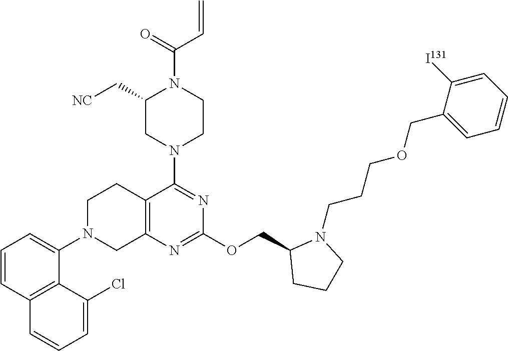 |
| B-2 | 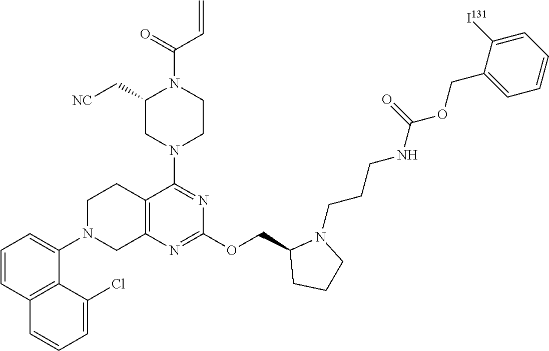 |
| B-3 | 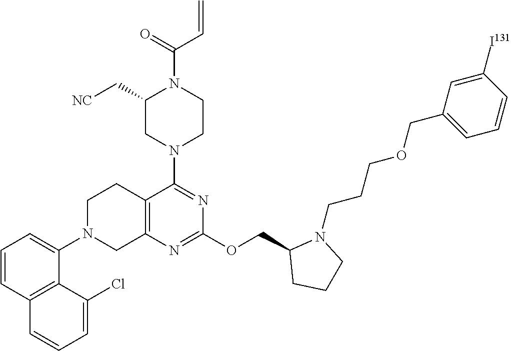 |
| B-4 | 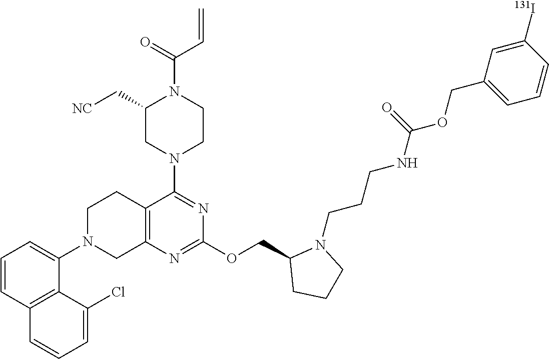 [SMILES: C=CC(=O)N1CCN(c2nc(OC[C@@H]3CCCN3CCCNC(=O)OCc3cccc([131I])c3)nc3c2CCN(c2cccc4cccc(Cl)c24)C3)C[C@@H]1CC#N] |
| B-5 | 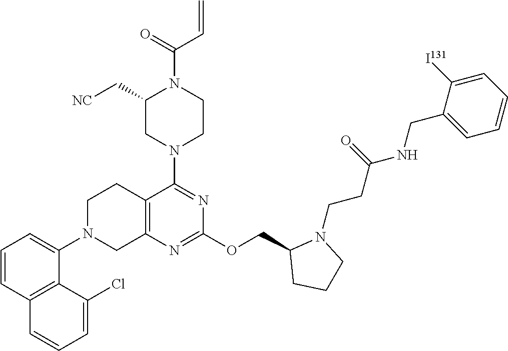 |
| B-6 | 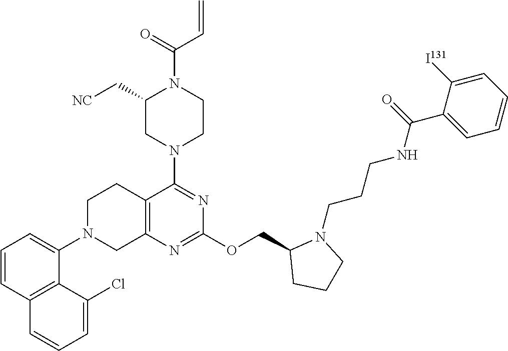 |
| B-7 | 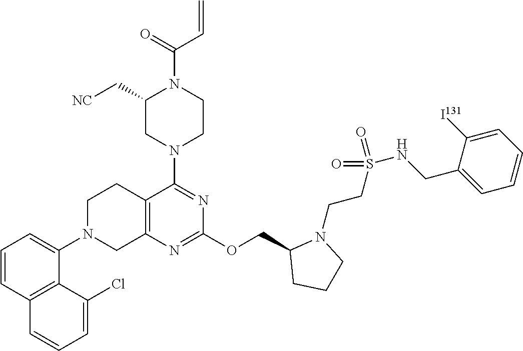 |
| B-8 | 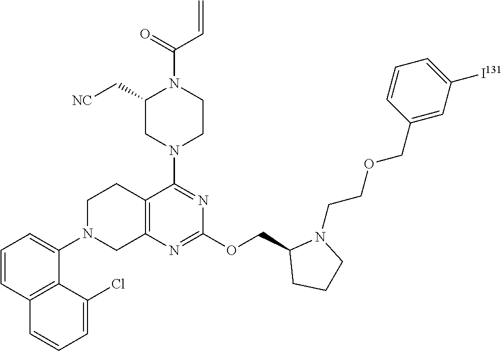 |
| B-9 | 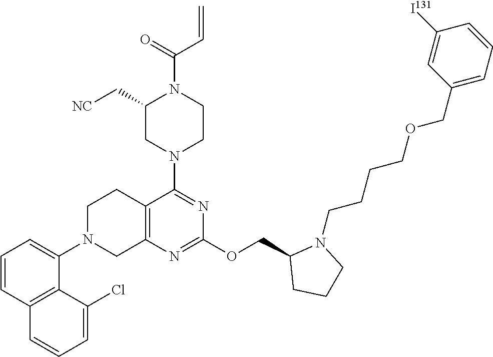 |
| B-10 | 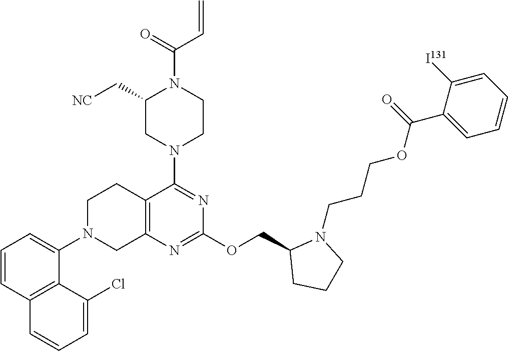 |
| B-11 | 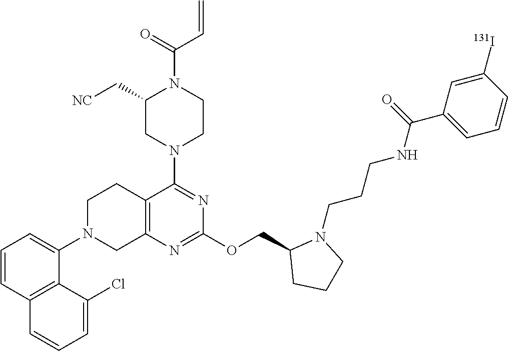 [SMILES: C=CC(=O)N1CCN(c2nc(OC[C@@H]3CCCN3CCCNC(=O)c3cccc([131I])c3)nc3c2CCN(c2cccc4cccc(Cl)c24)C3)C[C@@H]1CC#N] |
| B-12 | 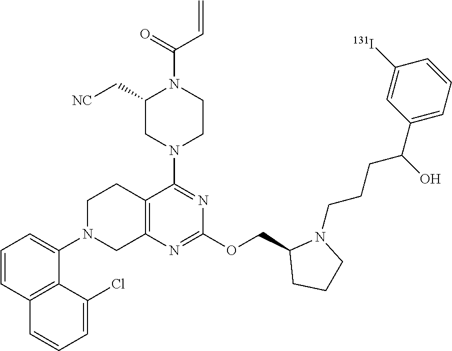 [SMILES: C=CC(=O)N1CCN(c2nc(OC[C@@H]3CCCN3CCCC(O)c3cccc([131I])c3)nc3c2CCN(c2cccc4cccc(Cl)c24)C3)C[C@@H]1CC#N] |
| B-13 | 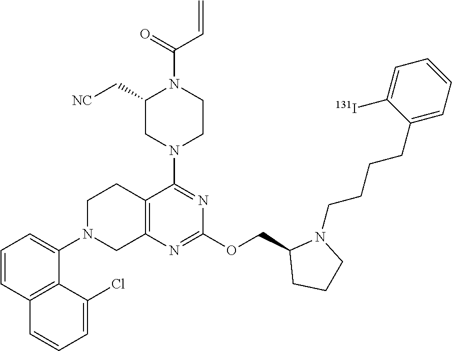 [SMILES: C=CC(=O)N1CCN(c2nc(OC[C@@H]3CCCN3CCCCc3ccccc3[131I])nc3c2CCN(c2cccc4cccc(Cl)c24)C3)C[C@@H]1CC#N] |
| B-14 | 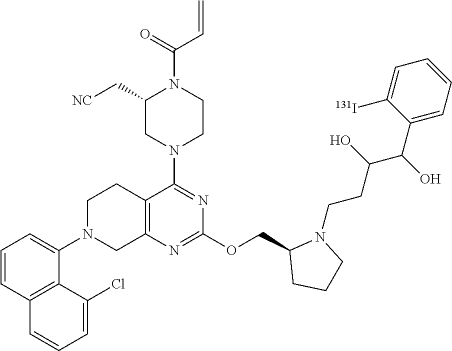 [SMILES: C=CC(=O)N1CCN(c2nc(OC[C@@H]3CCCN3CCC(O)C(O)c3ccccc3[131I])nc3c2CCN(c2cccc4cccc(Cl)c24)C3)C[C@@H]1CC#N] |
| B-15 | 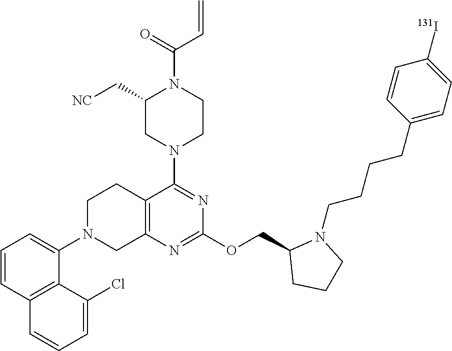 [SMILES: C=CC(=O)N1CCN(c2nc(OC[C@@H]3CCCN3CCCCc3ccc([131I])cc3)nc3c2CCN(c2cccc4cccc(Cl)c24)C3)C[C@@H]1CC#N] |
| B-16 | 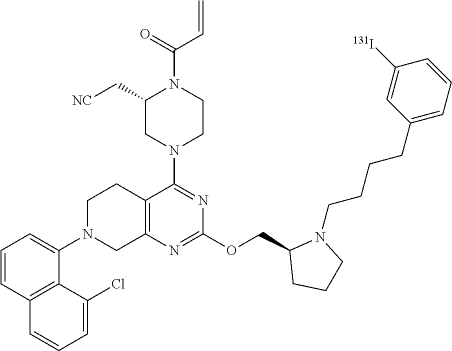 [SMILES: C=CC(=O)N1CCN(c2nc(OC[C@@H]3CCCN3CCCCc3cccc([131I])c3)nc3c2CCN(c2cccc4cccc(Cl)c24)C3)C[C@@H]1CC#N] |
| B-17 | 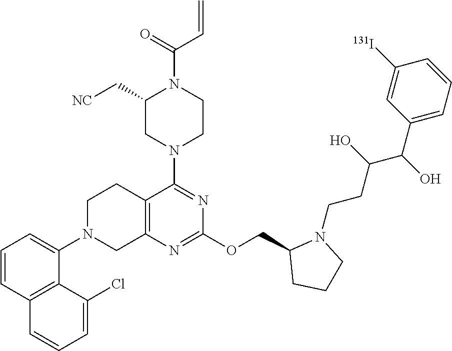 [SMILES: C=CC(=O)N1CCN(c2nc(OC[C@@H]3CCCN3CCC(O)C(O)c3cccc([131I])c3)nc3c2CCN(c2cccc4cccc(Cl)c24)C3)C[C@@H]1CC#N] |
| B-18 | 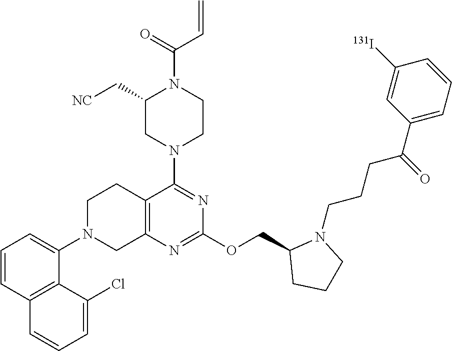 [SMILES: C=CC(=O)N1CCN(c2nc(OC[C@@H]3CCCN3CCCC(=O)c3cccc([131I])c3)nc3c2CCN(c2cccc4cccc(Cl)c24)C3)C[C@@H]1CC#N] |
| B-19 | 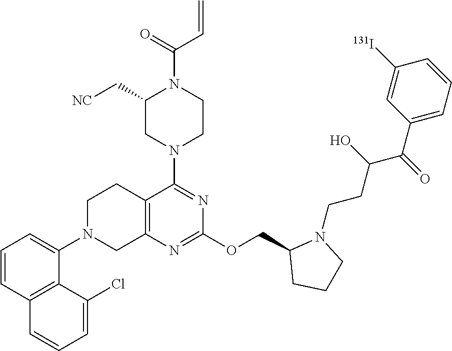 [SMILES: C=CC(=O)N1CCN(c2nc(OC[C@@H]3CCCN3CCC(O)C(=O)c3cccc([131I])c3)nc3c2CCN(c2cccc4cccc(Cl)c24)C3)C[C@@H]1CC#N] |
| B-20 | 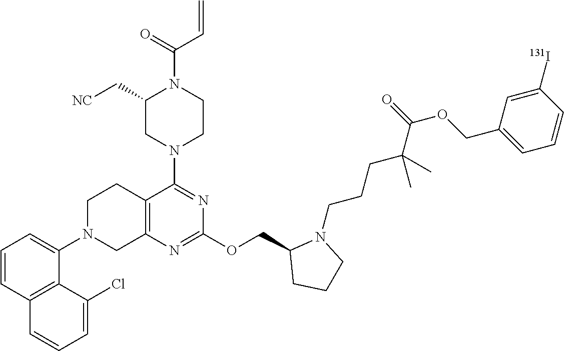 [SMILES: C=CC(=O)N1CCN(c2nc(OC[C@@H]3CCCN3CCCC(C)(C)C(=O)OCc3cccc([131I])c3)nc3c2CCN(c2cccc4cccc(Cl)c24)C3)C[C@@H]1CC#N] |
| B-21 | 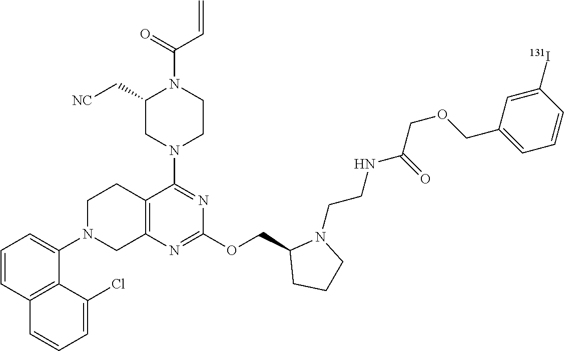 [SMILES: C=CC(=O)N1CCN(c2nc(OC[C@@H]3CCCN3CCNC(=O)COCc3cccc([131I])c3)nc3c2CCN(c2cccc4cccc(Cl)c24)C3)C[C@@H]1CC#N] |
| B-22 | 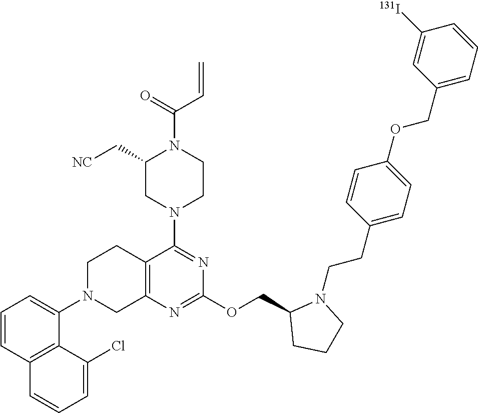 [SMILES: C=CC(=O)N1CCN(c2nc(OC[C@@H]3CCCN3CCc3ccc(OCc4cccc([131I])c4)cc3)nc3c2CCN(c2cccc4cccc(Cl)c24)C3)C[C@@H]1CC#N] |
| B-23 | 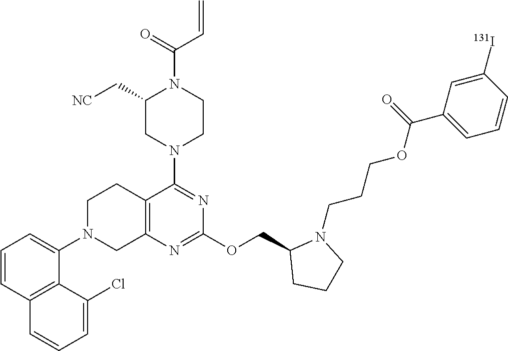 [SMILES: C=CC(=O)N1CCN(c2nc(OC[C@@H]3CCCN3CCCOC(=O)c3cccc([131I])c3)nc3c2CCN(c2cccc4cccc(Cl)c24)C3)C[C@@H]1CC#N] |
| B-24 | 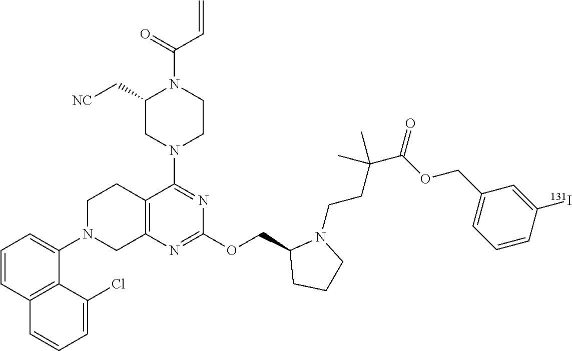 [SMILES: C=CC(=O)N1CCN(c2nc(OC[C@@H]3CCCN3CCC(C)(C)C(=O)OCc3cccc([131I])c3)nc3c2CCN(c2cccc4cccc(Cl)c24)C3)C[C@@H]1CC#N] |
| B-25 | 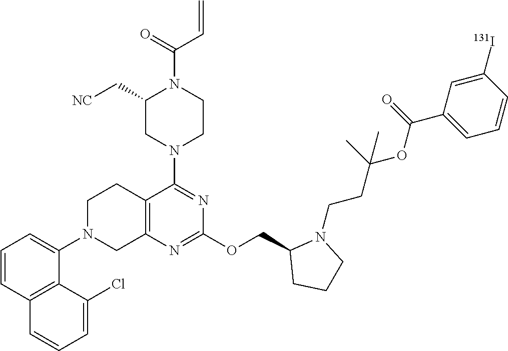 [SMILES: C=CC(=O)N1CCN(c2nc(OC[C@@H]3CCCN3CCC(C)(C)OC(=O)c3cccc([131I])c3)nc3c2CCN(c2cccc4cccc(Cl)c24)C3)C[C@@H]1CC#N] |
| B-26 | 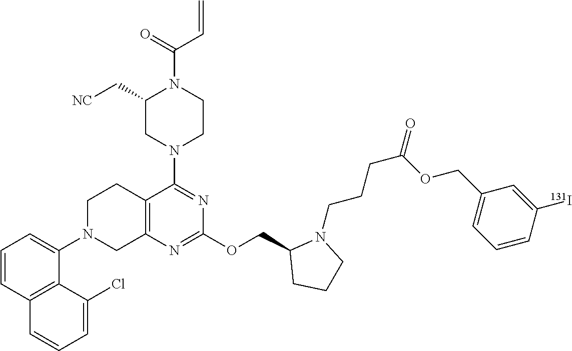 [SMILES: C=CC(=O)N1CCN(c2nc(OC[C@@H]3CCCN3CCCC(=O)OCc3cccc([131I])c3)nc3c2CCN(c2cccc4cccc(Cl)c24)C3)C[C@@H]1CC#N] |

TABLE 4C

| Compound | |
|---|---|
| ID | Structure |
| C-1 | 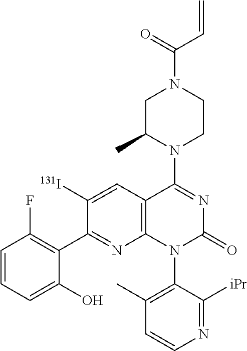 [SMILES: C=CC(=O)N1CCN(c2nc(=O)n(-c3c(C)ccnc3C(C)C)c3nc(-c4c(O)cccc4F)c([131I])cc23)[C@@H](C)C1] |
| C-2 | 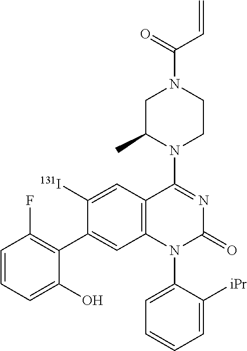 [SMILES: C=CC(=O)N1CCN(c2nc(=O)n(-c3ccccc3C(C)C)c3cc(-c4c(O)cccc4F)c([131I])cc23)[C@@H](C)C1] |
| C-3 | 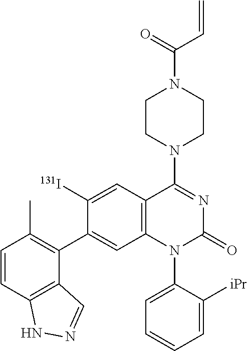 [SMILES: C=CC(=O)N1CCN(c2nc(=O)n(-c3ccccc3C(C)C)c3cc(-c4c(C)ccc5[nH]ncc45)c([131I])cc23)CC1] |
| C-4 | 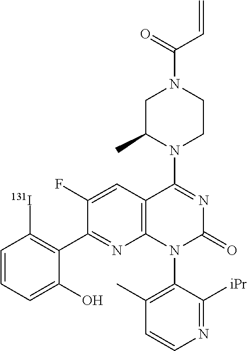 [SMILES: C=CC(=O)N1CCN(c2nc(=O)n(-c3c(C)ccnc3C(C)C)c3nc(-c4c(O)cccc4[131I])c(F)cc23)[C@@H](C)C1] |
| C-5 | 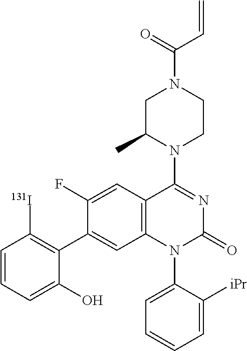 [SMILES: C=CC(=O)N1CCN(c2nc(=O)n(-c3ccccc3C(C)C)c3cc(-c4c(O)cccc4[131I])c(F)cc23)[C@@H](C)C1] |

TABLE 4D

| Com- | |
|---|---|
| pound | |
| ID | Structure |
| D-1 | 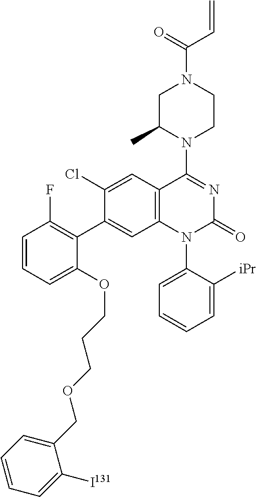 |
| D-2 | 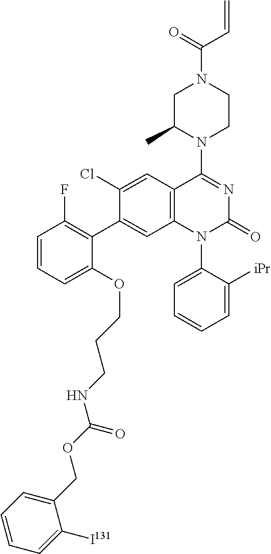 |
| D-3 | 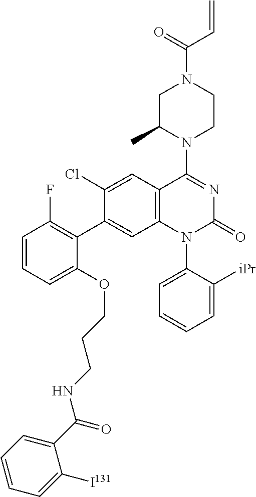 |
| D-4 | 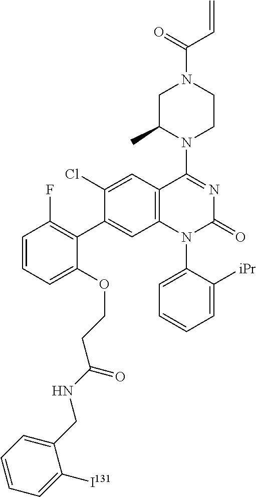 |
| D-5 | 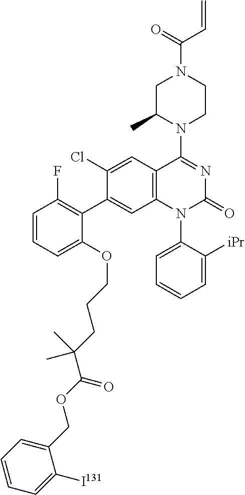 |
| D-6 | 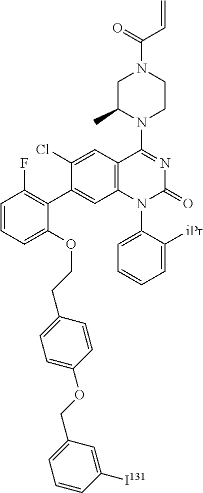 |
| D-7 | 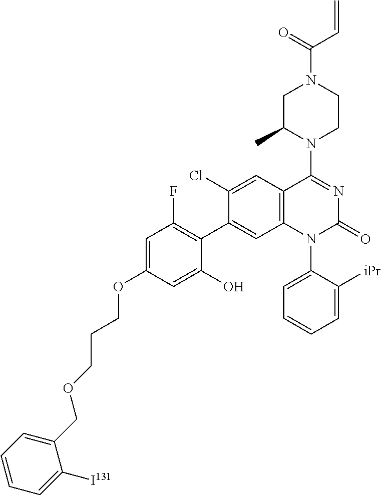 |
| D-8 | 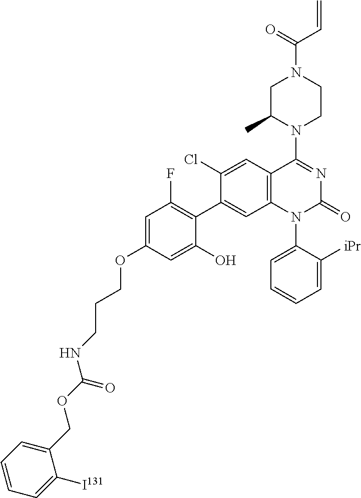 |
| D-9 | 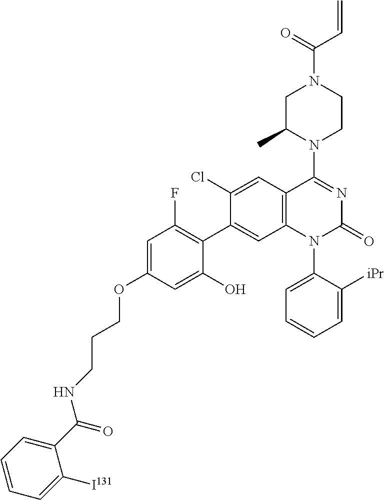 |
| D-10 | 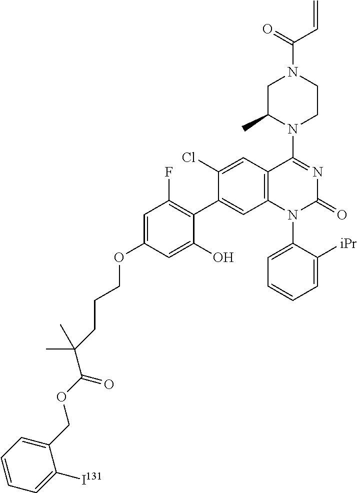 |

Provided herein are radiopharmaceutical conjugates and pharmaceutical compositions comprising the conjugates. The conjugates and compositions can be useful for treating cancer. The conjugates and compositions can also be useful in imaging and disease diagnosis.

In one aspect, described herein is a conjugate that comprises a targeting ligand that binds to an intracellular mutated KRAS protein, optionally a linker, and a metal chelator that is configured to bind with a radionuclide. In some embodiments, the targeting ligand can form an irreversible covalent bond to a KRAS protein. In some embodiments, the KRAS protein is mutated. In some embodiments, the KRAS mutation comprises a glycine to cysteine mutation at amino acid residue 12 (G12C mutation). In some embodiments, the conjugate descried herein forms a bond with the KRAS protein at G12C position. In some embodiments, the conjugate comprises a radionuclide such as ²²⁵Ac bound to the metal chelator.

In some embodiments, described herein is a conjugate comprising: (a) a targeting ligand that covalently binds a mutated KRAS protein at G12C position. (b) a linker that covalently attaches the targeting ligand to the metal chelator, and (c) a metal chelator configured to bind with a radionuclide. The targeting ligand can form a covalent bond with the mutated KRAS protein at G12C position. In some embodiments, the conjugate comprises a radionuclide such as ²²⁵Ac bound to the metal chelator.

In some embodiments, provided herein is a conjugate that has a structure of Formula (X).

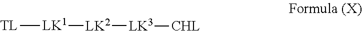

wherein,
TL represents the targeting ligand;
CHL represents the metal chelator, optionally bound to a radionuclide; and each of LK¹, LK², and LK³ is independently selected from substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₂-C₁₂ alkynylene, substituted or unsubstituted cycloalkylene, substituted or unsubstituted heterocycloalkylene, —(CH₂CHO)_{q}—, —(OCH₂CH₂)ₐ—O—, —S—, —S(═O)—, —S(═O)₂—, —S(═O)(═NR^{LK})—, —C(═O)—, —C(═N—OR)—, —C(═O)O—, —OC(═O)—. —C(═O)C(═O)—, —C(═O)NR^{LK}—, —NR^{LK}—C(═O)—, —OC(═O)NR—, —NR^{LK}C(O)O—, —NR^{LK}(═O)NR, —C(═O)NR^{LK}—, —C(═O)—, —S(═O)₂NR^{LK}—, —NRS(═O)₂—, —NR^{LK}—, —N(OR^{LK})—, and a bond;
each R^{LK} is independently hydrogen or substituted or unsubstituted C₁-C₆alkyl, and q is an integer selected from 1 to 10.

In some embodiments of Formula (X), the targeting ligand comprises MRTX849, AMG510, JNJ74699157, LY3499446, LY3537982, GDC6036, JDQ443, D1553, or a derivative thereof.

In some embodiments of Formula (X), the targeting ligand comprises a structure of

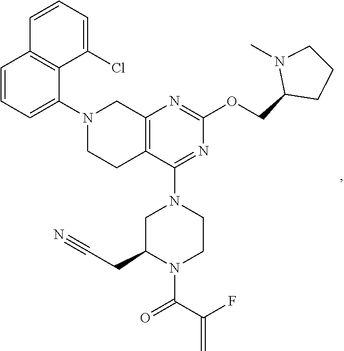 [SMILES: C=C(F)C(=O)N1CCN(c2nc(OC[C@@H]3CCCN3C)nc3c2CCN(c2cccc4cccc(Cl)c24)C3)C[C@@H]1CC#N]

wherein the structure is attached to the rest of the conjugate at any suitable position, e.g. through the 1-methylpyrrolidin-2-yl group.

In some embodiments of Formula (X), the targeting ligand comprises a structure of

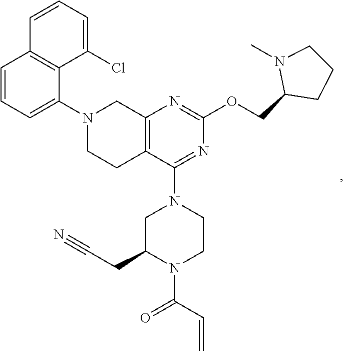 [SMILES: C=CC(=O)N1CCN(c2nc(OC[C@@H]3CCCN3C)nc3c2CCN(c2cccc4cccc(Cl)c24)C3)C[C@@H]1CC#N]

wherein the structure is attached to the rest of the conjugate at any suitable position, e.g. through the 1-methylpyrrolidin-2-yl group.

In some embodiments of Formula (X), the targeting ligand is

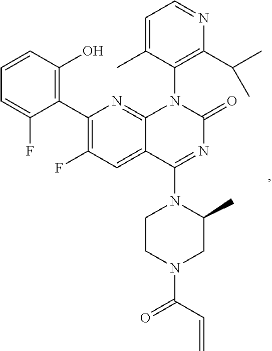 [SMILES: C=CC(=O)N1CCN(c2nc(=O)n(-c3c(C)ccnc3C(C)C)c3nc(-c4c(O)cccc4F)c(F)cc23)[C@@H](C)C1]

wherein the structure is attached to the rest of the conjugate at any suitable position (e.g., through group R²²).

In some embodiments of Formula (X), the targeting ligand is

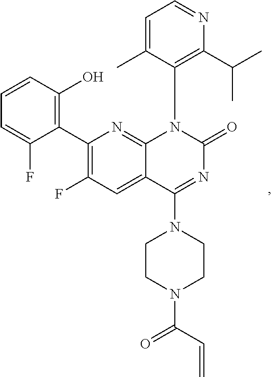 [SMILES: C=CC(=O)N1CCN(c2nc(=O)n(-c3c(C)ccnc3C(C)C)c3nc(-c4c(O)cccc4F)c(F)cc23)CC1]

wherein the structure is attached to the rest of the conjugate at any suitable position (e.g., through group R²²).

In some embodiments of Formula (X), the targeting ligand is

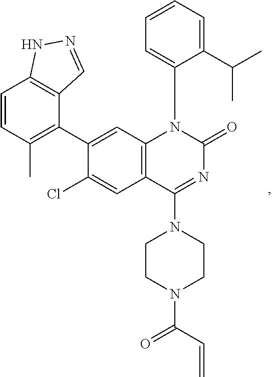 [SMILES: C=CC(=O)N1CCN(c2nc(=O)n(-c3ccccc3C(C)C)c3cc(-c4c(C)ccc5[nH]ncc45)c(Cl)cc23)CC1]

wherein the structure is attached to the rest of the conjugate at any suitable position (e.g., through group R²²).

In some embodiments, the targeting ligand TL has a structure disclosed herein. For example, TL can have a structure of Formula (III), a structure of Formula (IV), or a salt, solvate or derivative thereof.

In some embodiments, a conjugate of Formula (X) has a structure of Formula (X-III):

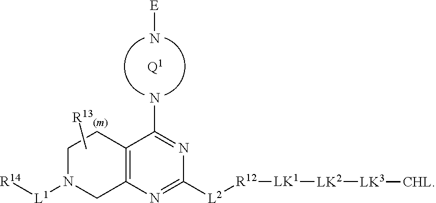

In some embodiments, a conjugate of Formula (X-III) is complexed with a radionuclide.

In some embodiments, a conjugate of Formula (X) has a structure of Formula (X-IV):

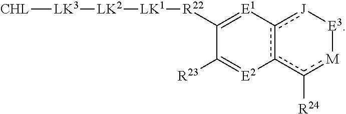

In some embodiments a conjugate of Formula (X-IV) is complexed with a radionuclide.

In some embodiments of Formula (X), Formula (X-III), or Formula (X-IV), LK¹ is substituted or unsubstituted C₁-C₁₂ alkylene. In some embodiments, LK¹ is substituted or unsubstituted C₁-C₃ alkylene. In some embodiments, LK¹ is

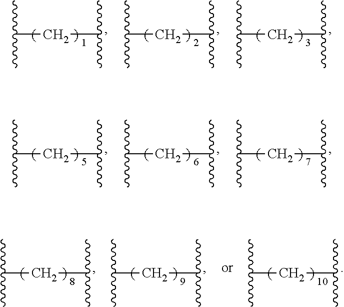

In some embodiments Formula (X), Formula (X-III), or Formula (X-IV). LK¹ is substituted or unsubstituted C₁-C₆ heteroalkylene. In some embodiments, LK¹ is substituted or unsubstituted C₂-C₆ heteroalkylene. In some embodiments, LK¹ is substituted or unsubstituted C₃-C₈ heteroalkylene.

In some embodiments Formula (X), Formula (X-III), or Formula (X-IV), LK¹ is C₁-C₆ alkylene. C₁-C₆ heteroalkylene, —(CH₂CH₂O)₁₋₆—, —(OCH₂CH₂)₁₋₆—, —O—, or —S—. In some embodiments, LK¹ is —(CH₂CH₂O)₁₋₆— or —(OCH₂CH₂)₁₋₆—. In some embodiments, LK¹ is —(CH₂CH₂O)₅—. In some embodiments, LK¹ is —(CH₂CH₂O)₂—. In some embodiments, LK¹ is —(CH₂CH₂O)₂—. In some embodiments, LK¹ is —(CH₂—CH₂O)₂—. In some embodiments, LK¹ is —CH₂CH₂O—. In some embodiments, LK¹ is —(OCH₂CH₂)₁—. In some embodiments, LK¹ is —(OCH₂CH₂)₄—. In some embodiments, LK¹ is —(OCH₂—CH₂)₃—. In some embodiments, LK¹ is —(OCH₂CH₂)—. In some embodiments, LK¹ is —OCH₂CH₂—.

In some embodiments Formula (X), Formula (X-III), or Formula (X-IV), LK¹ is —NH—. In some embodiments, LK¹ is a bond.

In some embodiments Formula (X), Formula (X-III), or Formula (X-IV), LK² is C₁-C₆ alkylene, C₁-C₆ heteroalkylene, —(CH₂CH—O)₁₋₃—, —(OCH₂CH₂)₁₋₃—, —O—, or —S—. In some embodiments, LK² is —(CH₂CH₂O)₁₋₃- or —(OCH₂CH₂)₁₋₃—. In some embodiments, LK² is —(CH₂CH₂O)—. In some embodiments, LK² is —CH₂CH₂O—. In some embodiments, LK² is —(OCH₂CH₂)₂—. In some embodiments, LK² is —OCH₂CH₂—.

In some embodiments Formula (X), Formula (X-III), or Formula (X-IV), LK² is substituted or unsubstituted cycloalkylene, or substituted or unsubstituted heterocycloalkylene. In some embodiments, LK² is monocyclic. In some embodiments, LK² is 3-6 membered substituted or unsubstituted heterocycloalkylene. In some embodiments, LK² is

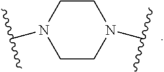 [SMILES: *N1CCN(*)CC1]

In some embodiments Formula (X), Formula (X-III), or Formula (X-IV), LK² is O—, —S—, —S(═O)—, —S(═O)₂—, —S(═O)(═NR^{LK})—, —C(═O)—, —C(═NR^{LK})—, —C(═O)O—, —OC(═O)—, —C(═OC(═O)—, —C(O)NR^{LK}—, —NR^{LK}C(═O)—, —OC(═O)NR^{LK}—, —NR^{LK}C(═O)O—, —NR^{LK}C(═O)NR^{LK}—, —C(═O)NR^{LK}C(═O)—, —S(═O)₂NR^{LK}—, —NR^{LK}S(═O)₂, or —NR^{LK}—.

In some embodiments Formula (X), Formula (X-III), or Formula (X-IV). LK² is —O—. In some embodiments, LK² is —C(═O)NR^{LK}— or —NR^{LK}C(═O)—. In some embodiments, LK² is —C(═O)NH—. In some embodiments. LK² is —NHC(═O)—.

In some embodiments Formula (X), Formula (X-III), or Formula (X-IV), LK² is a bond.

In some embodiments Formula (X), Formula (X-III), or Formula (X-IV), LK³ is substituted or unsubstituted C₁-C₁₂ alkylene. In some embodiments, LK³ is substituted or unsubstituted C₁-C₃ alkylene. In some embodiments LK³ is

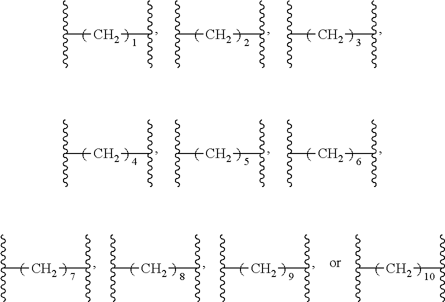

In some embodiments Formula (X), Formula (X-III), or Formula (X-IV). LK³ is substituted or unsubstituted C₁-C₆ heteroalkylene. In some embodiments, LK³ is substituted or unsubstituted C₂-C₆ heteroalkylene. In some embodiments, LK is substituted or unsubstituted C₂-C₈ heteroalkylene.

In some embodiments Formula (X), Formula (X-III), or Formula (X-IV). LK³ is C₁-C₆ alkylene, C₁-C₆heteroalkylene, —(CH₂CH₂O)₁₋₃—, —(OCH₂CH₂)₁₋₃—, —O—, or —S—. In some embodiments, LK³ is substituted or unsubstituted C₁-C₁₂ heteroalkylene. In some embodiments, LK³ is substituted or unsubstituted C₂-C₆ heteroalkylene. In some embodiments, LK³ is substituted or unsubstituted C₃-C₈ heteroalkylene.

In some embodiments Formula (X), Formula (X-III), or Formula (X-IV), LK³ is a bond.

Exemplary configurations of conjugates of Formula (X), Formula (X-III), and Formula (X-IV) described herein are illustrated in Table 5A. Table 5B, Table 5C and Table 5D. In some embodiments, provided herein are conjugates comprising a structure of Table 5A or Table 5C, and a radionuclide (e.g., ²²⁵Ac, ¹⁷⁷Lu). In some embodiments, a conjugate describe herein contains a radioactive isotope. e.g., conjugates 225Ac-CHL-001 to 225Ac-CHL-018 in Table 5B. In some embodiments, a conjugate describe herein does not contain a radioactive isotope, e.g., conjugates CHL-001—CHL-018 in Table 5A.

TABLE 5A
Structures of Exemplary Conjugates

| Com- | |
|---|---|
| pound | |
| ID | Structure |
| CHL-001 | 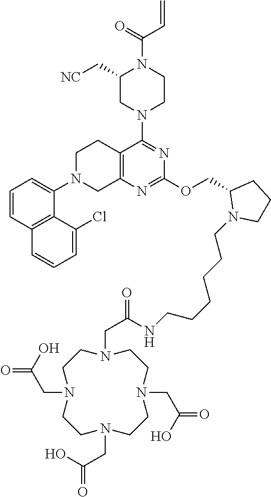 [SMILES: C=CC(=O)N1CCN(c2nc(OC[C@@H]3CCCN3CCCCCCNC(=O)CN3CCN(CC(=O)O)CCN(CC(=O)O)CCN(CC(=O)O)CC3)nc3c2CCN(c2cccc4cccc(Cl)c24)C3)C[C@@H]1CC#N] |
| CHL-002 | 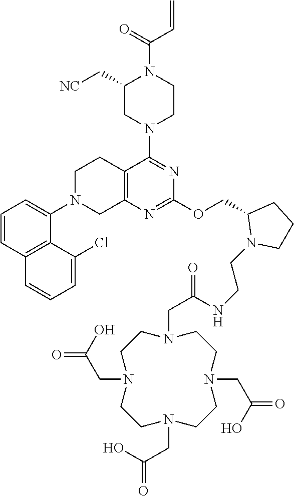 [SMILES: C=CC(=O)N1CCN(c2nc(OC[C@@H]3CCCN3CCNC(=O)CN3CCN(CC(=O)O)CCN(CC(=O)O)CCN(CC(=O)O)CC3)nc3c2CCN(c2cccc4cccc(Cl)c24)C3)C[C@@H]1CC#N] |
| CHL-003 | 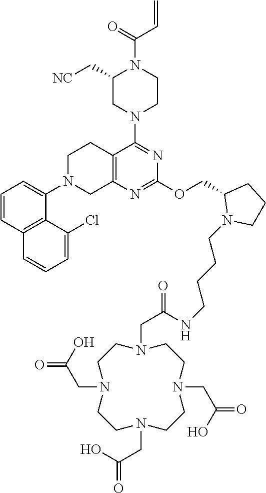 [SMILES: C=CC(=O)N1CCN(c2nc(OC[C@@H]3CCCN3CCCCNC(=O)CN3CCN(CC(=O)O)CCN(CC(=O)O)CCN(CC(=O)O)CC3)nc3c2CCN(c2cccc4cccc(Cl)c24)C3)C[C@@H]1CC#N] |
| CHL-004 | 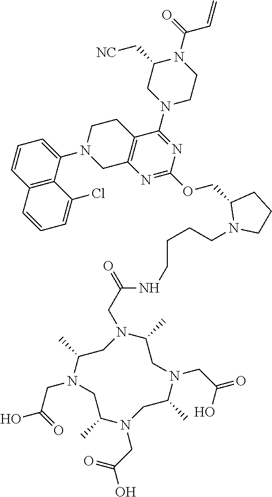 [SMILES: C=CC(=O)N1CCN(c2nc(OC[C@@H]3CCCN3CCCCNC(=O)CN3C[C@@H](C)N(CC(=O)O)C[C@@H](C)N(CC(=O)O)C[C@@H](C)N(CC(=O)O)C[C@H]3C)nc3c2CCN(c2cccc4cccc(Cl)c24)C3)C[C@@H]1CC#N] |
| CHL-005 | 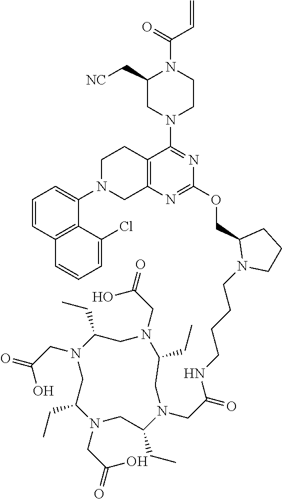 [SMILES: C=CC(=O)N1CCN(c2nc(OC[C@H]3CCCN3CCCCNC(=O)CN3C[C@@H](CC)N(CC(=O)O)C[C@@H](CC)N(CC(=O)O)C[C@@H](CC)N(CC(=O)O)C[C@H]3CC)nc3c2CCN(c2cccc4cccc(Cl)c24)C3)C[C@H]1CC#N] |
| CHL-006 | 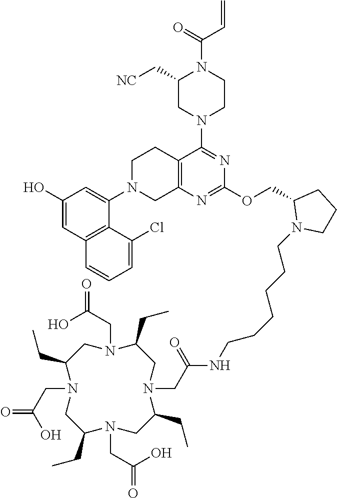 [SMILES: C=CC(=O)N1CCN(c2nc(OC[C@@H]3CCCN3CCCCCCNC(=O)CN3C[C@H](CC)N(CC(=O)O)C[C@H](CC)N(CC(=O)O)C[C@H](CC)N(CC(=O)O)C[C@@H]3CC)nc3c2CCN(c2cc(O)cc4cccc(Cl)c24)C3)C[C@@H]1CC#N] |
| CHL-007 | 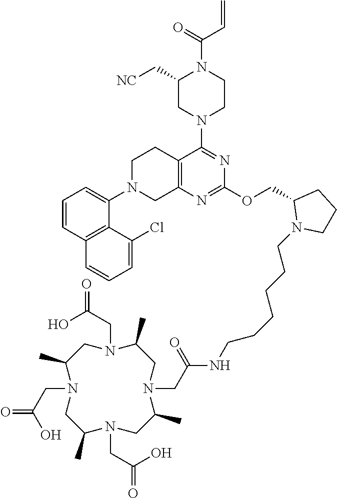 [SMILES: C=CC(=O)N1CCN(c2nc(OC[C@@H]3CCCN3CCCCCCNC(=O)CN3C[C@H](C)N(CC(=O)O)C[C@H](C)N(CC(=O)O)C[C@H](C)N(CC(=O)O)C[C@@H]3C)nc3c2CCN(c2cccc4cccc(Cl)c24)C3)C[C@@H]1CC#N] |
| CHL 008 | 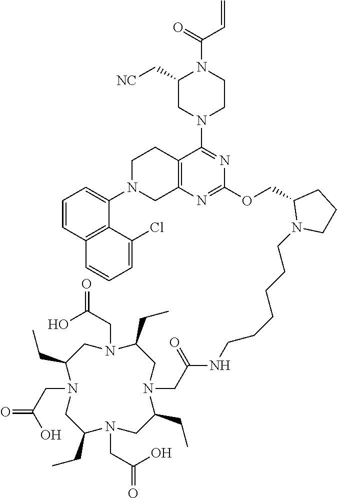 [SMILES: C=CC(=O)N1CCN(c2nc(OC[C@@H]3CCCN3CCCCCCNC(=O)CN3C[C@H](CC)N(CC(=O)O)C[C@H](CC)N(CC(=O)O)C[C@H](CC)N(CC(=O)O)C[C@@H]3CC)nc3c2CCN(c2cccc4cccc(Cl)c24)C3)C[C@@H]1CC#N] |
| CHL 009 | 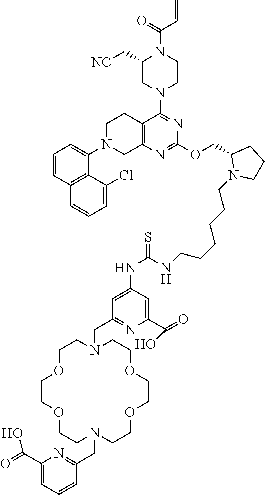 [SMILES: C=CC(=O)N1CCN(c2nc(OC[C@@H]3CCCN3CCCCCCNC(=S)Nc3cc(CN4CCOCCOCCN(Cc5cccc(C(=O)O)n5)CCOCCOCC4)nc(C(=O)O)c3)nc3c2CCN(c2cccc4cccc(Cl)c24)C3)C[C@@H]1CC#N] |
| CHL 010 | 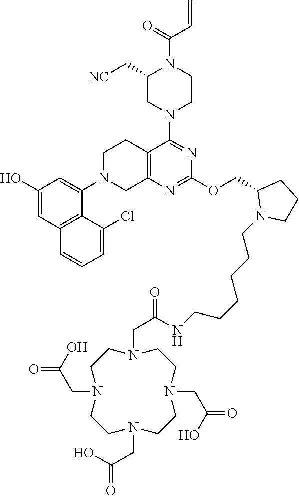 [SMILES: C=CC(=O)N1CCN(c2nc(OC[C@@H]3CCCN3CCCCCCNC(=O)CN3CCN(CC(=O)O)CCN(CC(=O)O)CCN(CC(=O)O)CC3)nc3c2CCN(c2cc(O)cc4cccc(Cl)c24)C3)C[C@@H]1CC#N] |
| CHL-011 | 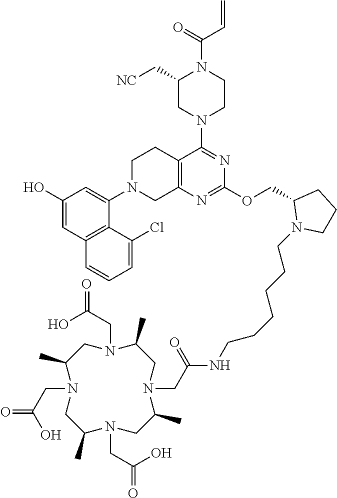 [SMILES: C=CC(=O)N1CCN(c2nc(OC[C@@H]3CCCN3CCCCCCNC(=O)CN3C[C@H](C)N(CC(=O)O)C[C@H](C)N(CC(=O)O)C[C@H](C)N(CC(=O)O)C[C@@H]3C)nc3c2CCN(c2cc(O)cc4cccc(Cl)c24)C3)C[C@@H]1CC#N] |
| CHL-012 | 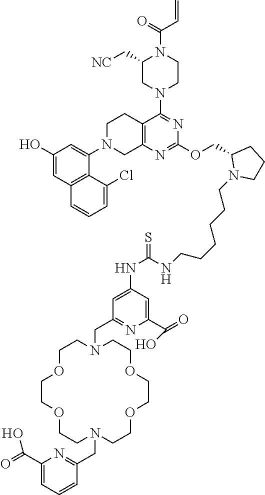 [SMILES: C=CC(=O)N1CCN(c2nc(OC[C@@H]3CCCN3CCCCCCNC(=S)Nc3cc(CN4CCOCCOCCN(Cc5cccc(C(=O)O)n5)CCOCCOCC4)nc(C(=O)O)c3)nc3c2CCN(c2cc(O)cc4cccc(Cl)c24)C3)C[C@@H]1CC#N] |
| CHL-013 | 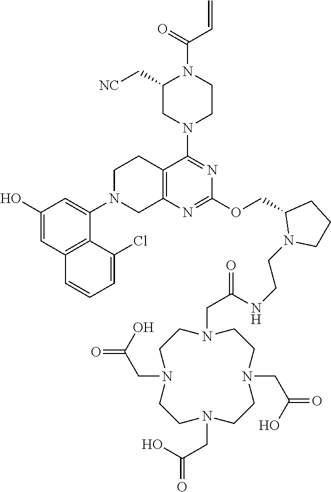 [SMILES: C=CC(=O)N1CCN(c2nc(OC[C@@H]3CCCN3CCNC(=O)CN3CCN(CC(=O)O)CCN(CC(=O)O)CCN(CC(=O)O)CC3)nc3c2CCN(c2cc(O)cc4cccc(Cl)c24)C3)C[C@@H]1CC#N] |
| CHL-014 | 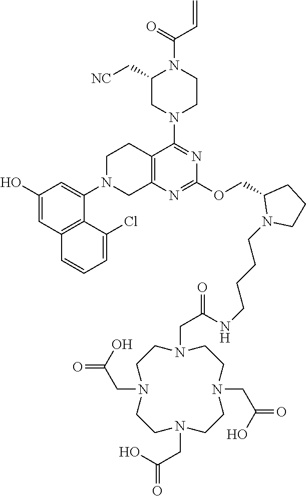 [SMILES: C=CC(=O)N1CCN(c2nc(OC[C@@H]3CCCN3CCCCNC(=O)CN3CCN(CC(=O)O)CCN(CC(=O)O)CCN(CC(=O)O)CC3)nc3c2CCN(c2cc(O)cc4cccc(Cl)c24)C3)C[C@@H]1CC#N] |
| CHL-015 | 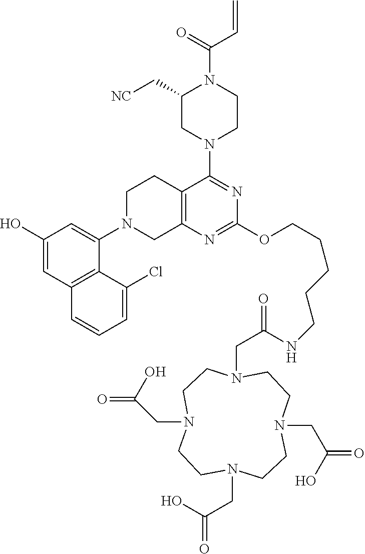 [SMILES: C=CC(=O)N1CCN(c2nc(OCCCCCNC(=O)CN3CCN(CC(=O)O)CCN(CC(=O)O)CCN(CC(=O)O)CC3)nc3c2CCN(c2cc(O)cc4cccc(Cl)c24)C3)C[C@@H]1CC#N] |
| CHL-016 | 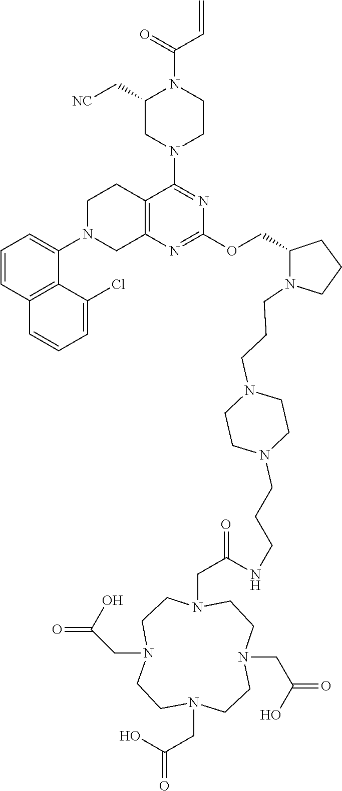 [SMILES: C=CC(=O)N1CCN(c2nc(OC[C@@H]3CCCN3CCCN3CCN(CCCNC(=O)CN4CCN(CC(=O)O)CCN(CC(=O)O)CCN(CC(=O)O)CC4)CC3)nc3c2CCN(c2cccc4cccc(Cl)c24)C3)C[C@@H]1CC#N] |
| CHL-017 | 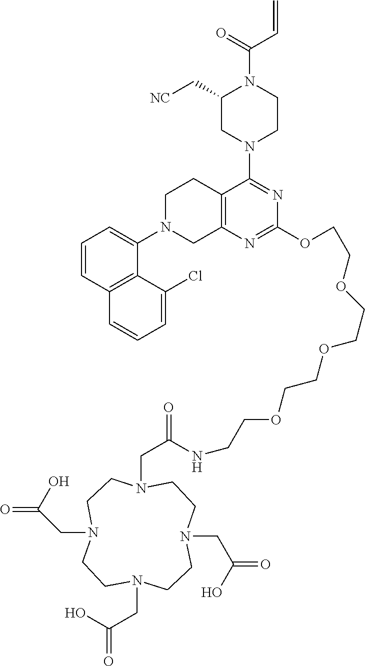 [SMILES: C=CC(=O)N1CCN(c2nc(OCCOCCOCCOCCNC(=O)CN3CCN(CC(=O)O)CCN(CC(=O)O)CCN(CC(=O)O)CC3)nc3c2CCN(c2cccc4cccc(Cl)c24)C3)C[C@@H]1CC#N] |
| CHL-018 | 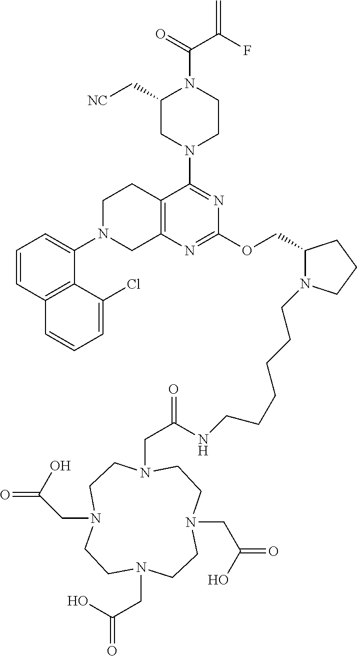 [SMILES: C=C(F)C(=O)N1CCN(c2nc(OC[C@@H]3CCCN3CCCCCCNC(=O)CN3CCN(CC(=O)O)CCN(CC(=O)O)CCN(CC(=O)O)CC3)nc3c2CCN(c2cccc4cccc(Cl)c24)C3)C[C@@H]1CC#N] |

TABLE 5B
Structures of Exemplary Conjugates

| Compound | |
|---|---|
| ID | Structure |
| 177Lu- CHL-001 | 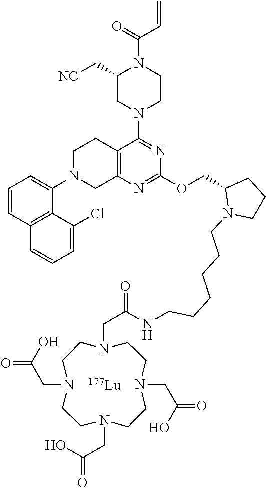 |
| 225Ac- CHL-001 | 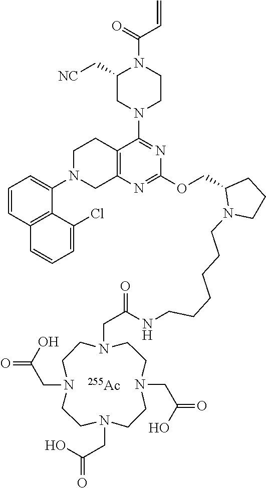 |
| 177Lu- CHL-002 | 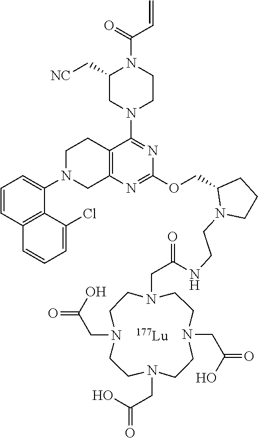 |
| 225Ac- CHL-002 | 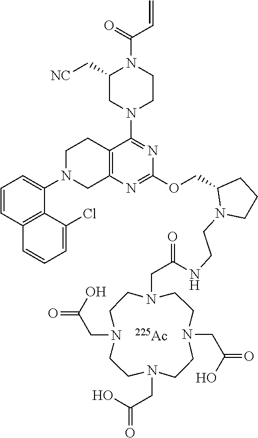 |
| 177Lu- CHL-003 | 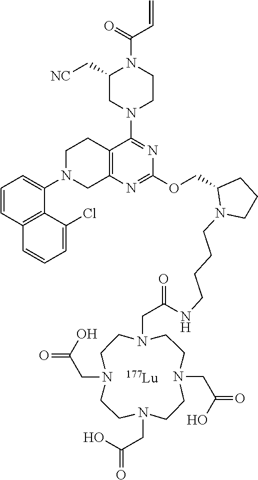 |
| 225Ac- CHL-003 | 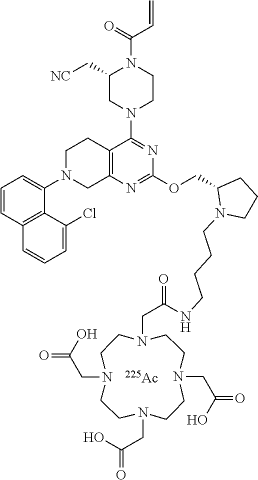 |
| 177Lu- CHL-004 | 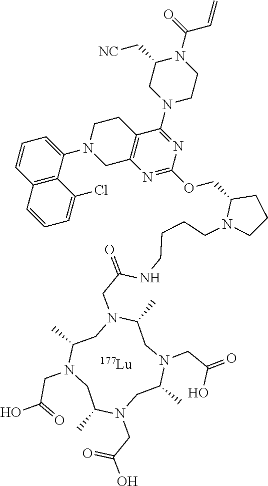 |
| 225Ac- CHL-004 | 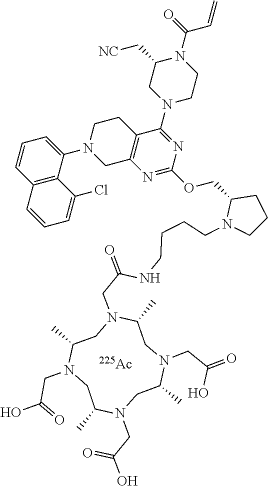 |
| 177Lu- CHL-005 | 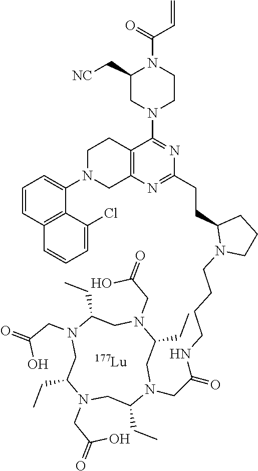 |
| 225Ac- CHL-005 | 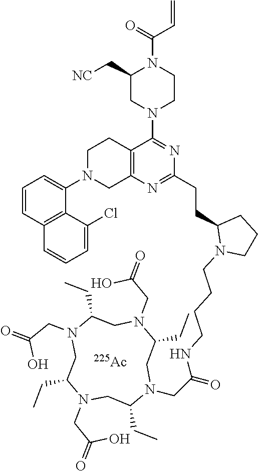 |
| 177Lu- CHL-006 | 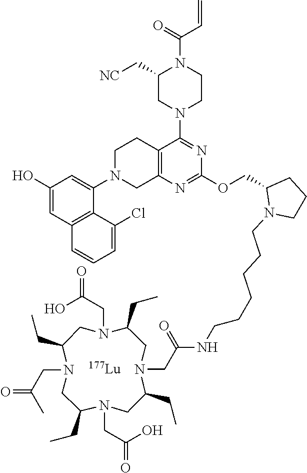 |
| 225Ac- CHL-006 | 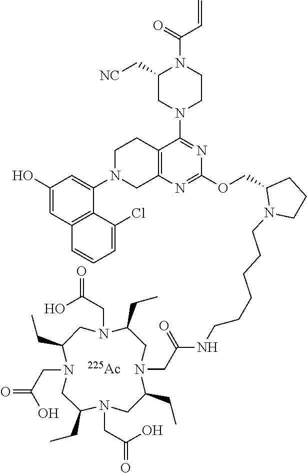 |
| 177Lu- CHL-007 | 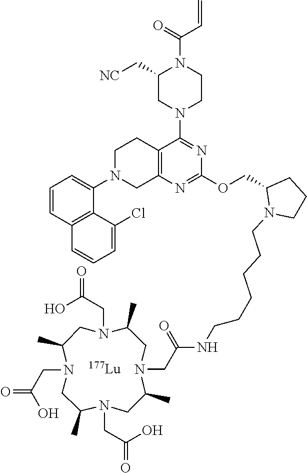 |
| 225Ac- CHL-007 | 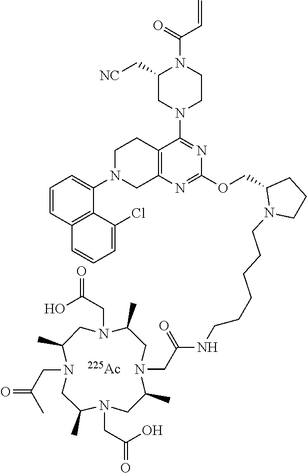 |
| 177Lu- CHL 008 | 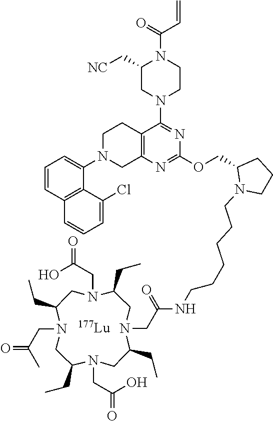 |
| 225Ac- CHL 008 | 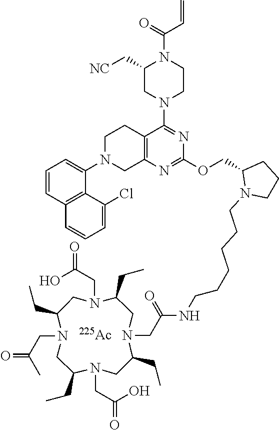 |
| 177Lu- CHL 009 | 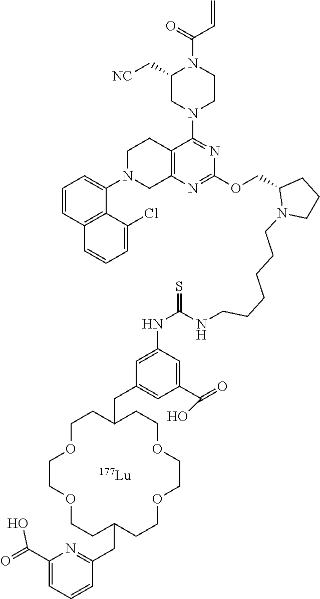 |
| 225Ac- CHL 009 | 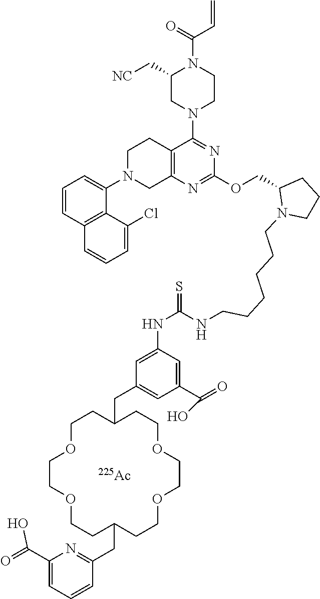 |
| 177Lu- CHL 010 | 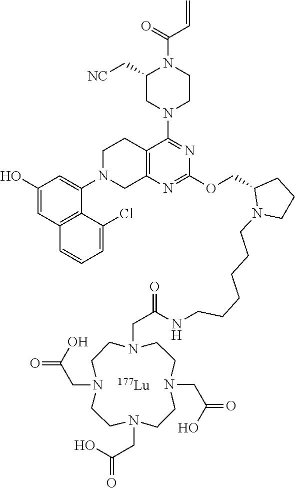 |
| 225Ac- CHL 010 | 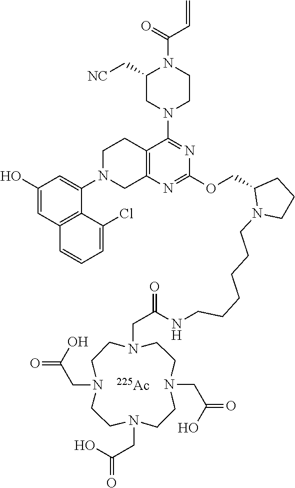 |
| 177Lu- CHL-011 | 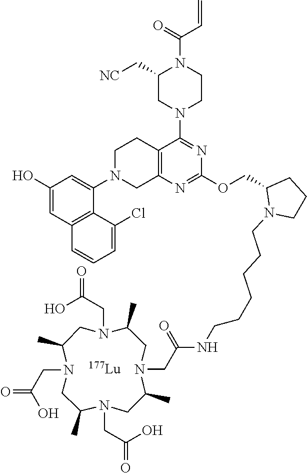 |
| 225Ac- CHL-011 | 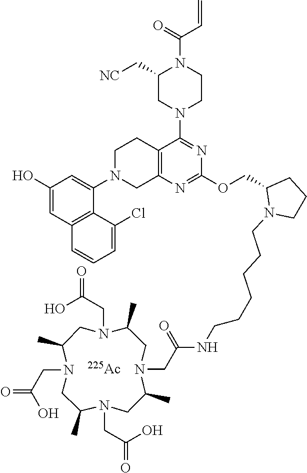 |
| 177Lu- CHL-012 | 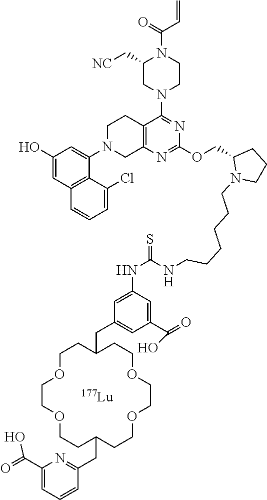 |
| 225Ac- CHL-012 | 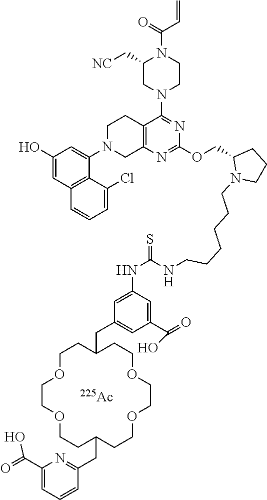 |
| 177Lu- CHL-013 | 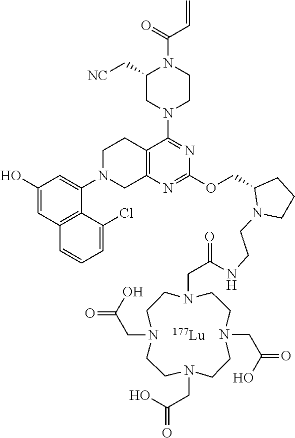 |
| 225Ac- CHL-013 | 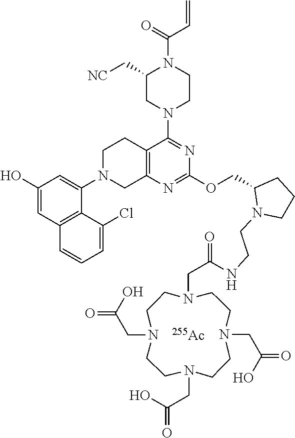 |
| 177Lu- CHL-014 | 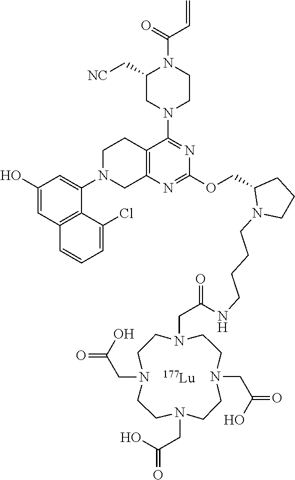 |
| 225Ac- CHL-014 | 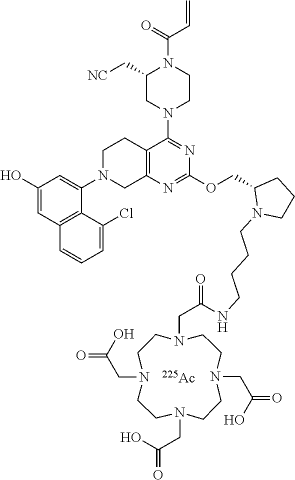 |
| 177Lu- CHL-015 | 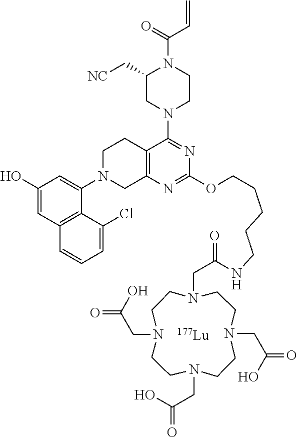 |
| 225Lu- CHL-015 | 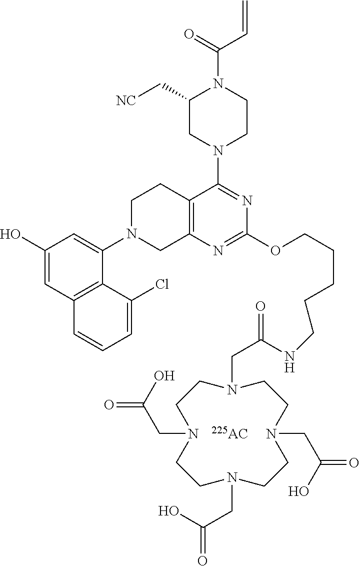 |
| 177Lu- CHL-016 | 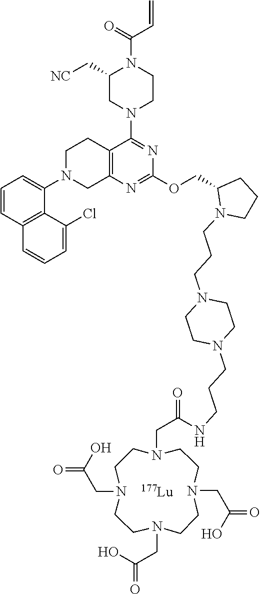 |
| 225Lu- CHL-016 | 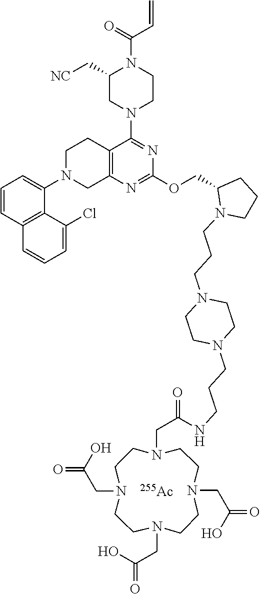 |
| 177Lu- CHL-017 | 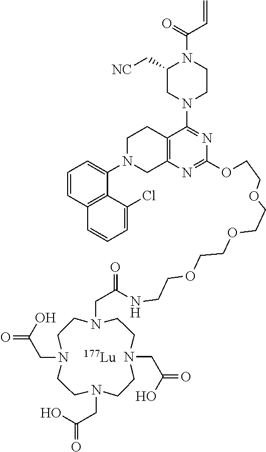 |
| 225Lu- CHL-017 | 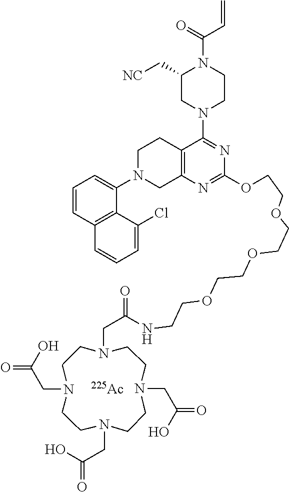 |
| 177Lu- CHL-018 | 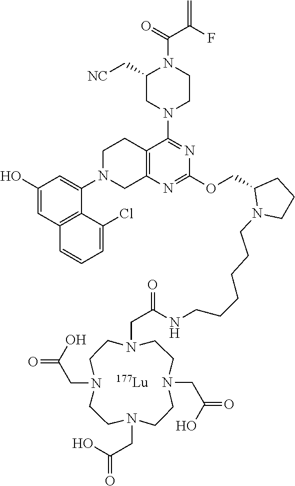 |
| 225Lu- CHL-018 | 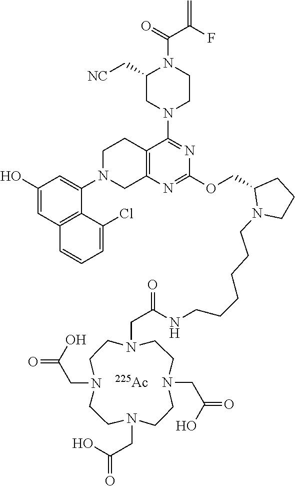 |

TABLE 5C
Structures of Exemplary Conjugates

| Compound | |
|---|---|
| ID | Structure |
| CHL-201 | 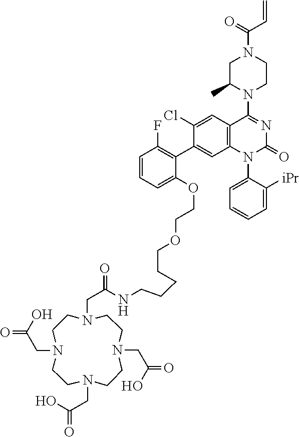 [SMILES: C=CC(=O)N1CCN(c2nc(=O)n(-c3ccccc3C(C)C)c3cc(-c4c(F)cccc4OCCOCCCCCNC(=O)CN4CCN(CC(=O)O)CCN(CC(=O)O)CCN(CC(=O)O)CC4)c(Cl)cc23)[C@@H](C)C1] |
| CHL-202 | 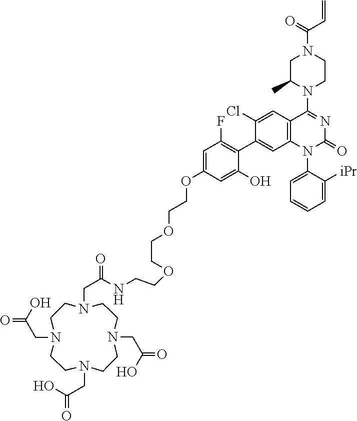 [SMILES: C=CC(=O)N1CCN(c2nc(=O)n(-c3ccccc3C(C)C)c3cc(-c4c(O)cc(OCCOCCOCCNC(=O)CN5CCN(CC(=O)O)CCN(CC(=O)O)CCN(CC(=O)O)CC5)cc4F)c(Cl)cc23)[C@@H](C)C1] |

TABLE 5D
Structures of Exemplary Conjugates

| Compound | |
|---|---|
| ID | Structure |
| 177Lu- CHL-201 | 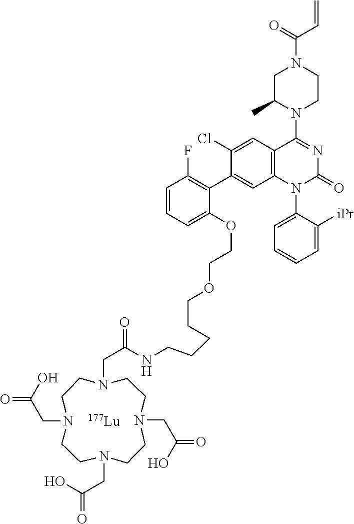 |
| 2225 Ac- CHL-201 | 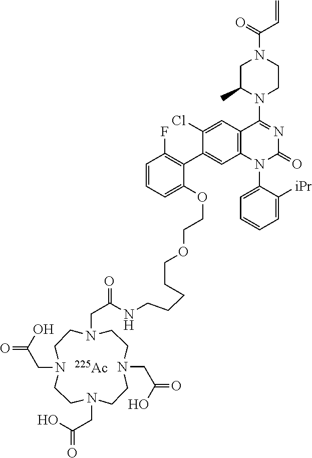 |
| 177Lu- CHL-202 | 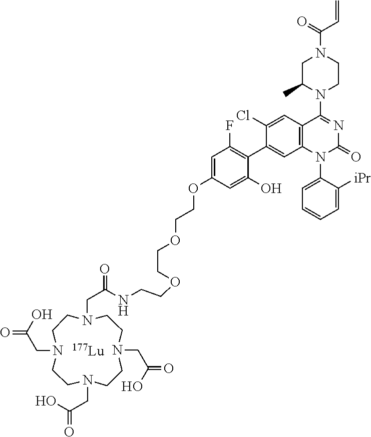 |
| 2225 Ac- CHL-202 | 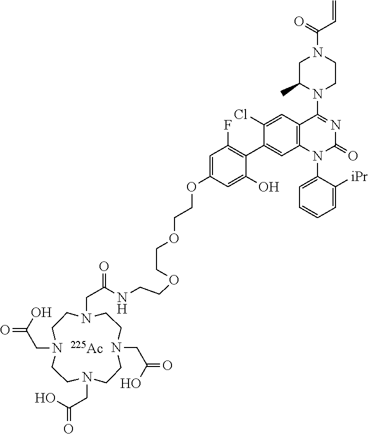 |

It is understood that the structures of conjugates in Tables 5A-5D are shown for illustration purposes. A person skilled in the art would appreciate that the bonding between the radionuclide (¹⁷⁷Lu or ²²⁵Ac) and the metal chelator in conjugates of Tables 5B and 5D is not shown.

A metal chelator such as DOTA can interact with a radionuclide (e.g., ¹⁷⁷Lu or ²²⁵Ac) via one or more functional groups and/or atoms. For example, a metal chelator can interact with a radionuclide via nitrogen and/or oxygen atoms. As another example, a metal chelator can interact with a radionuclide via carbonyl, carboxylic acid, amino, and/or amide groups of the metal chelator. In some embodiments, the interaction of a metal chelator and a radionuclide of the conjugates disclosed herein can be illustrated as

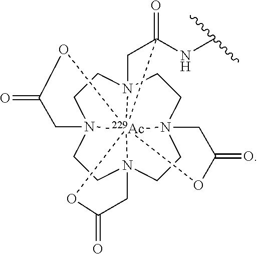

In some embodiments, the interaction of a metal chelator and a radionuclide of the conjugates disclosed herein can be illustrated as

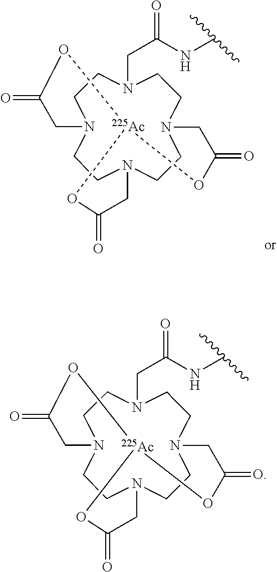

In some embodiments, the interaction of a metal chelator and a radionuclide of the conjugates disclosed herein can be illustrated as

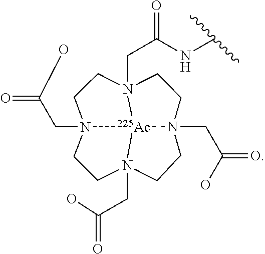

In some embodiments, the interaction of a metal chelator and a radionuclide of the conjugates disclosed herein can be illustrated as

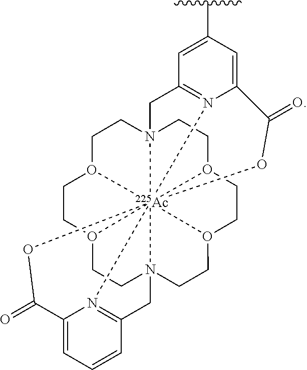

In some embodiments, the radionuclide exists in a positive oxidation state e.g., ²²⁵Ac, ¹⁷⁷Lu³⁺. In some embodiments, for example in certain aqueous conditions, the radionuclide exists in a salt form. e.g., as ²²⁵Ac³⁺, ¹⁷⁷Lu³⁺. In some embodiments, for example in certain acidic aqueous conditions, the radionuclide exists in a salt form, e.g., as ²²⁵Ac³⁺, ¹⁷⁷Lu³⁺. In some embodiments, the conjugate is in a salt form. In some embodiments, one or more of the carboxylic acid groups of the conjugate may exist as carboxy late anions. In some embodiments, one or more of the carboxylate anions of the conjugate may coordinate to the radionuclide. A person of ordinary skill would appreciate that the dissociation of an acid can depend on the pH value of the environment and its pK value. Accordingly, in some embodiments, a conjugate described herein can exist in a completely ionized, partially ionized or non-ionized form.

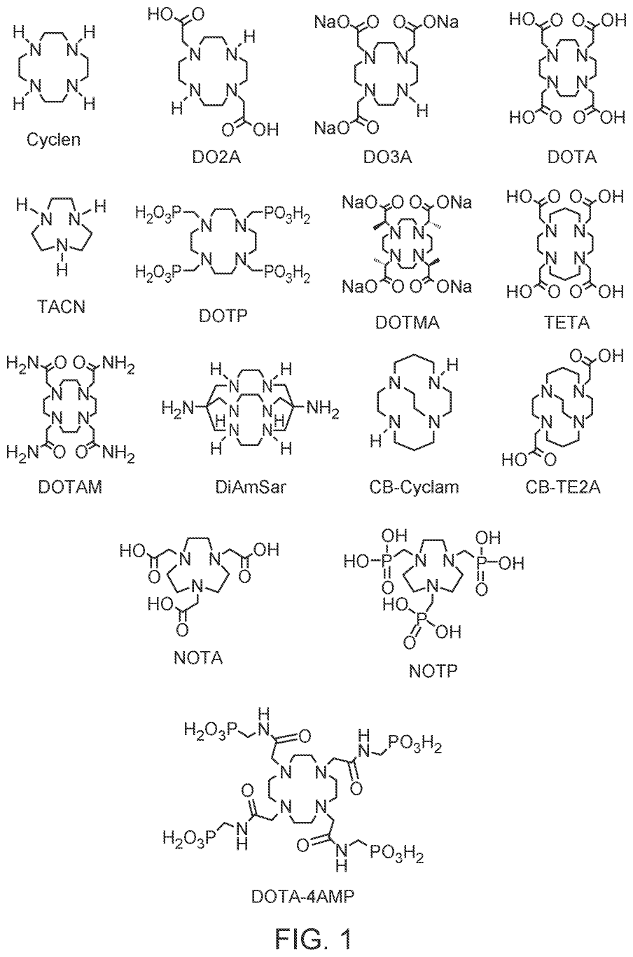

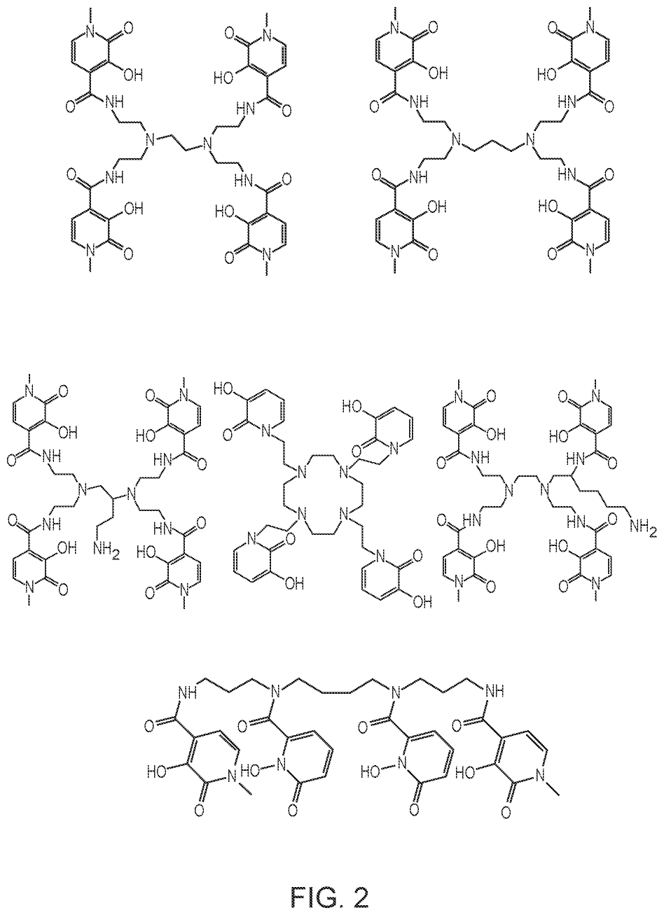

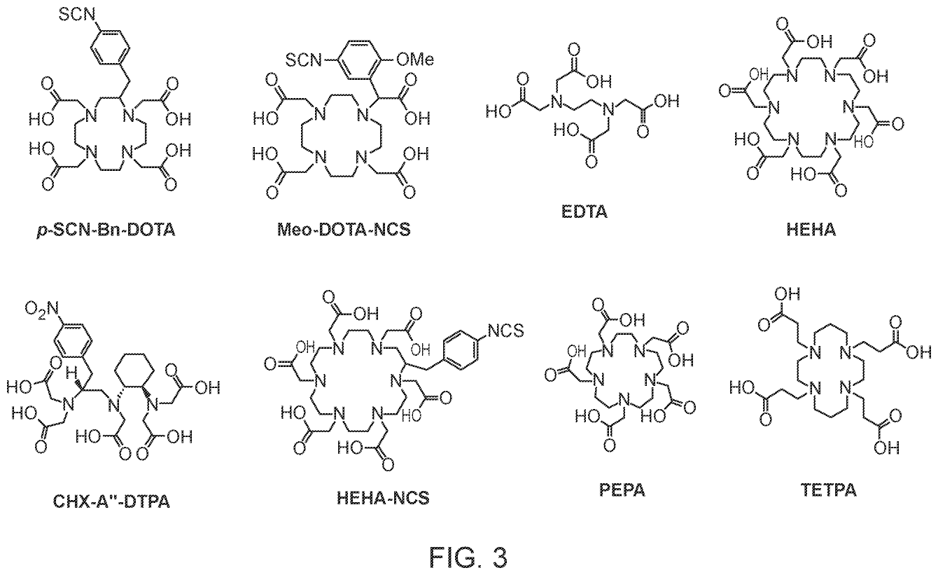

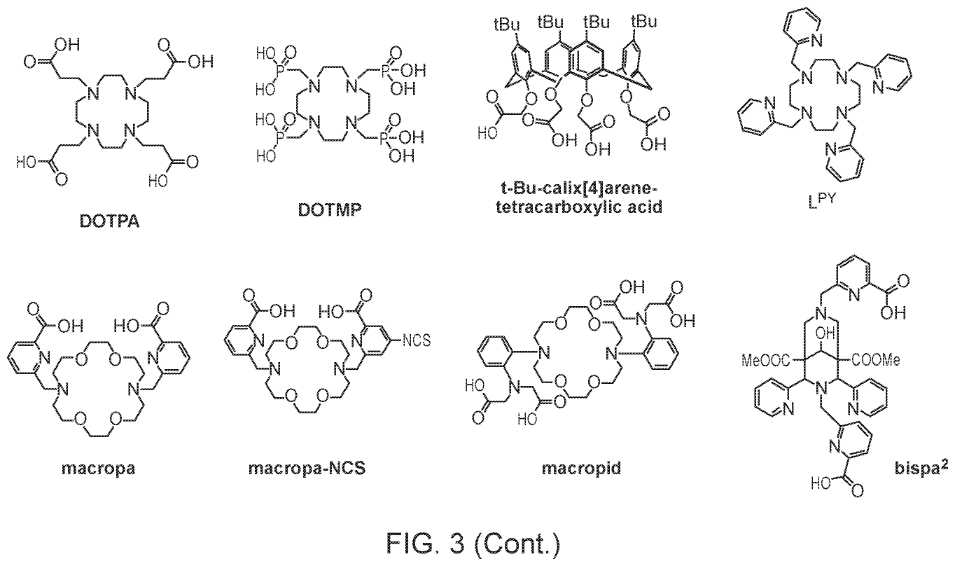

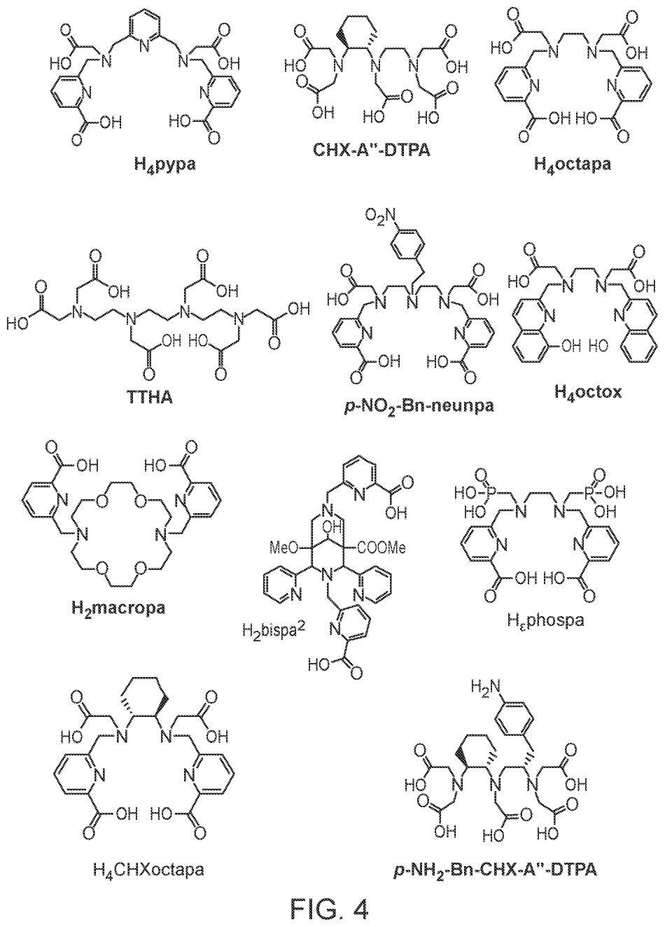

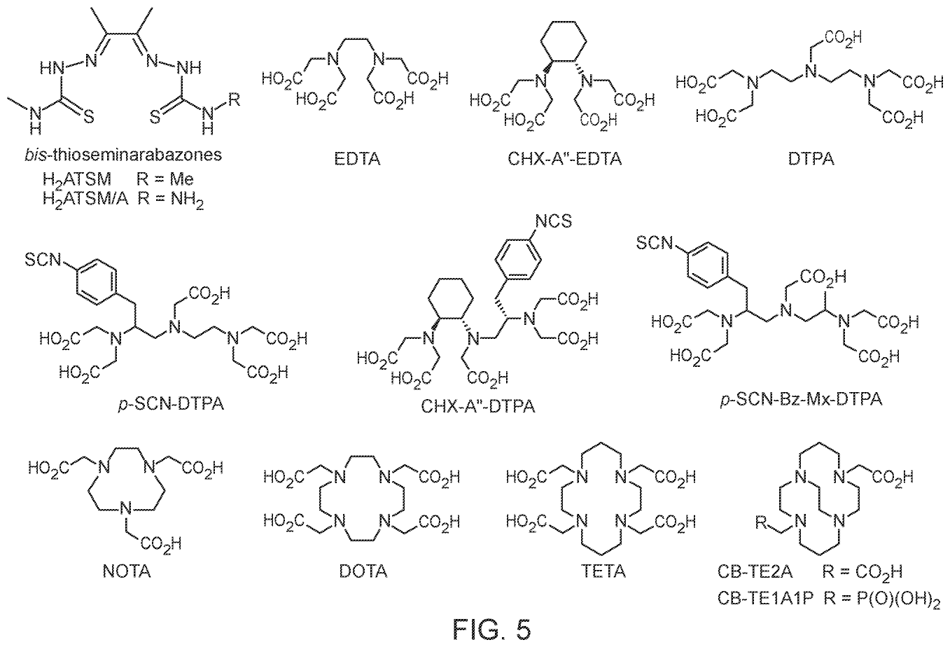

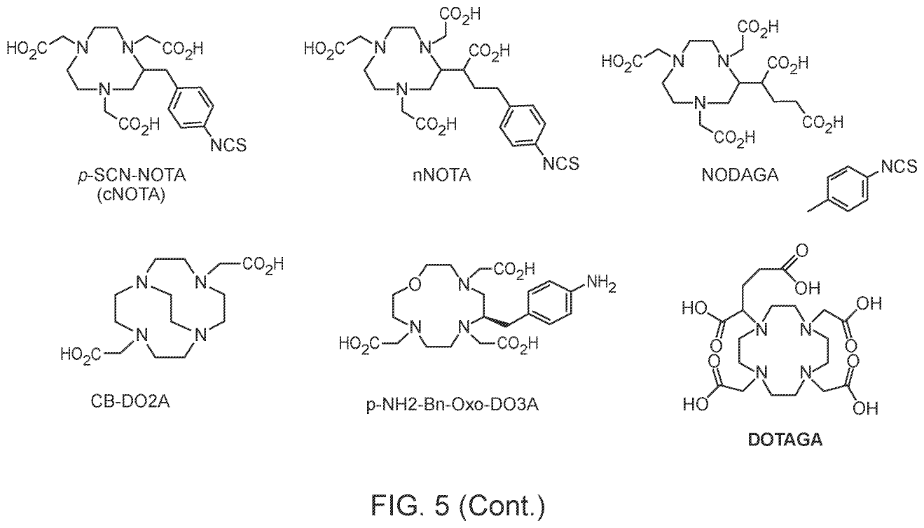

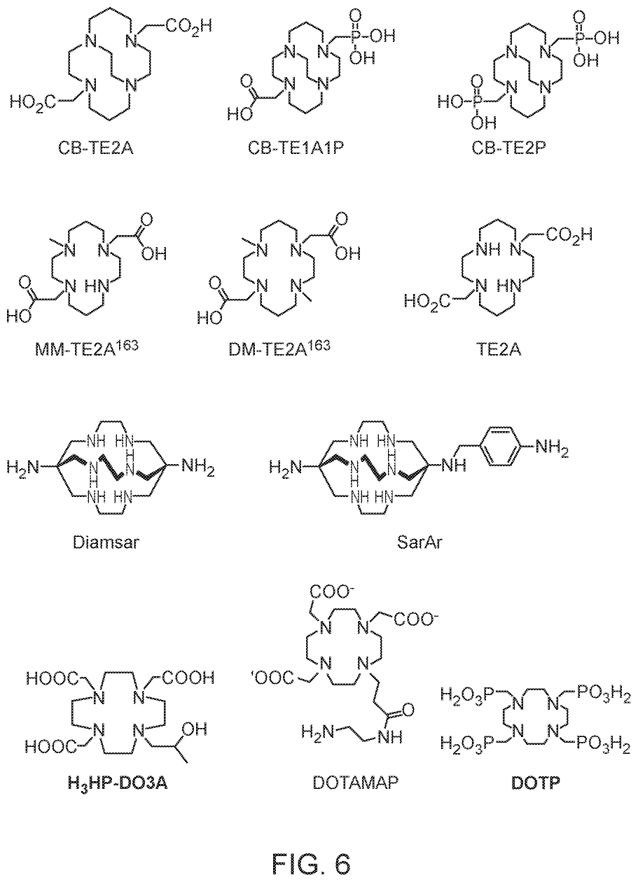

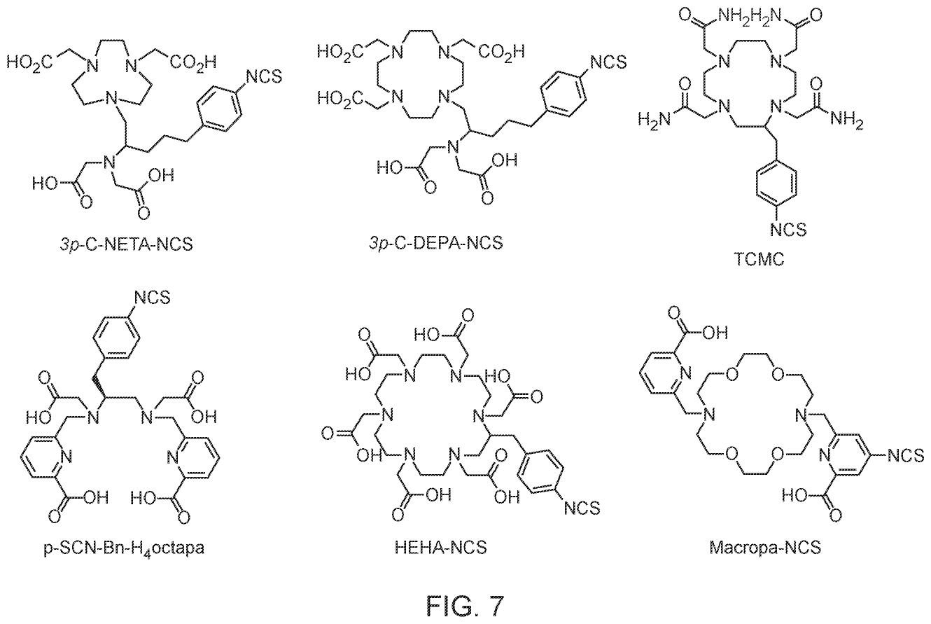

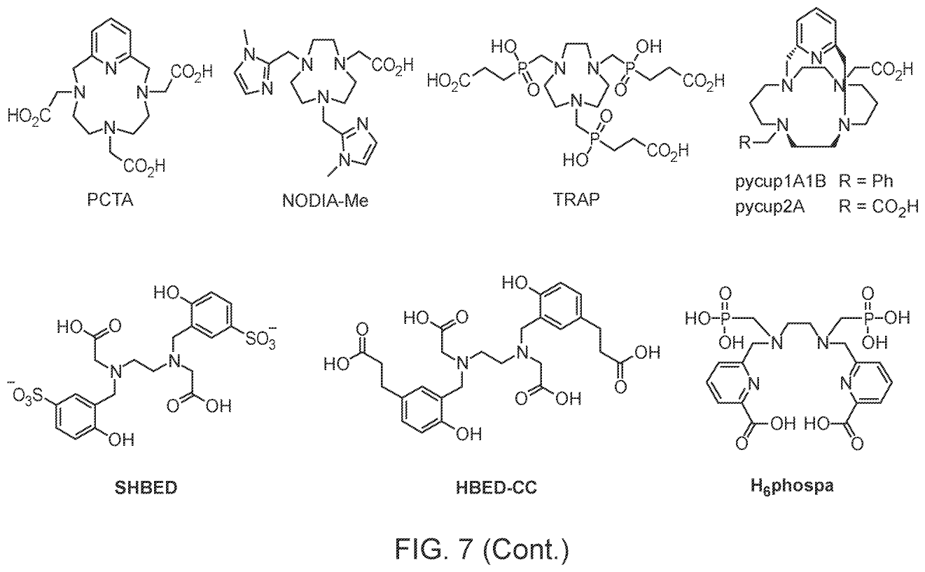

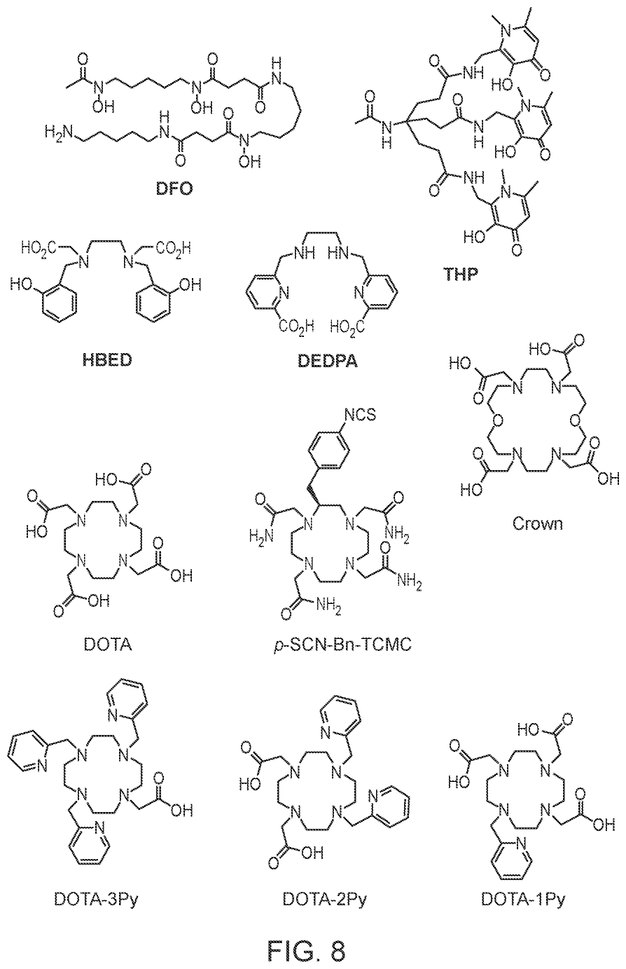

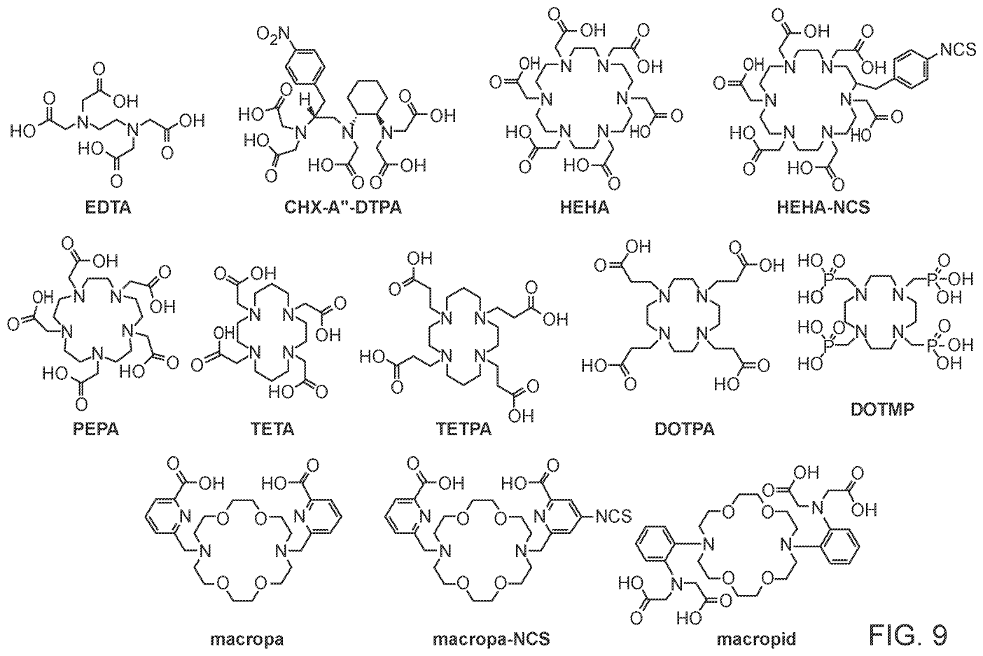

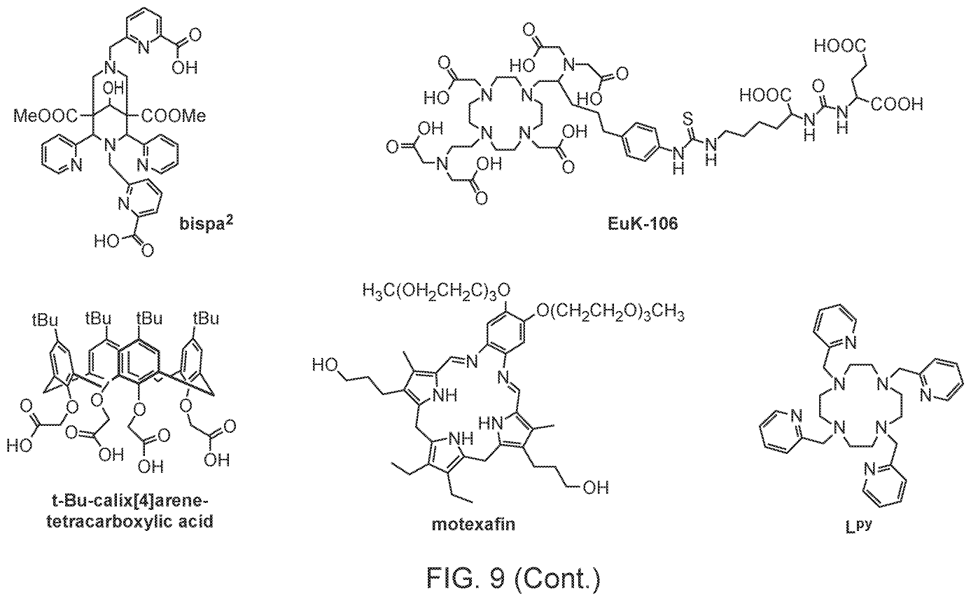

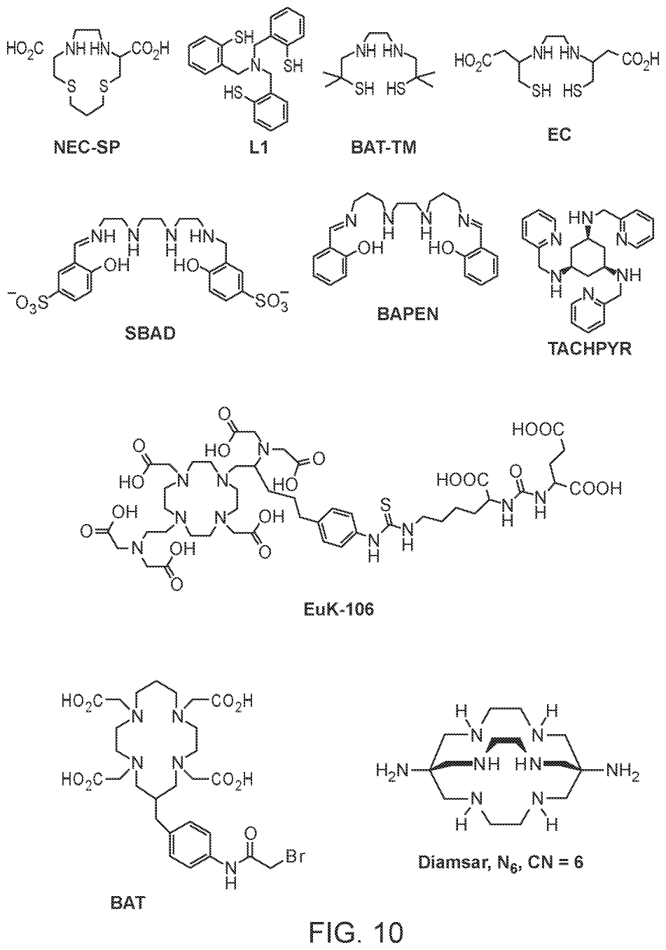

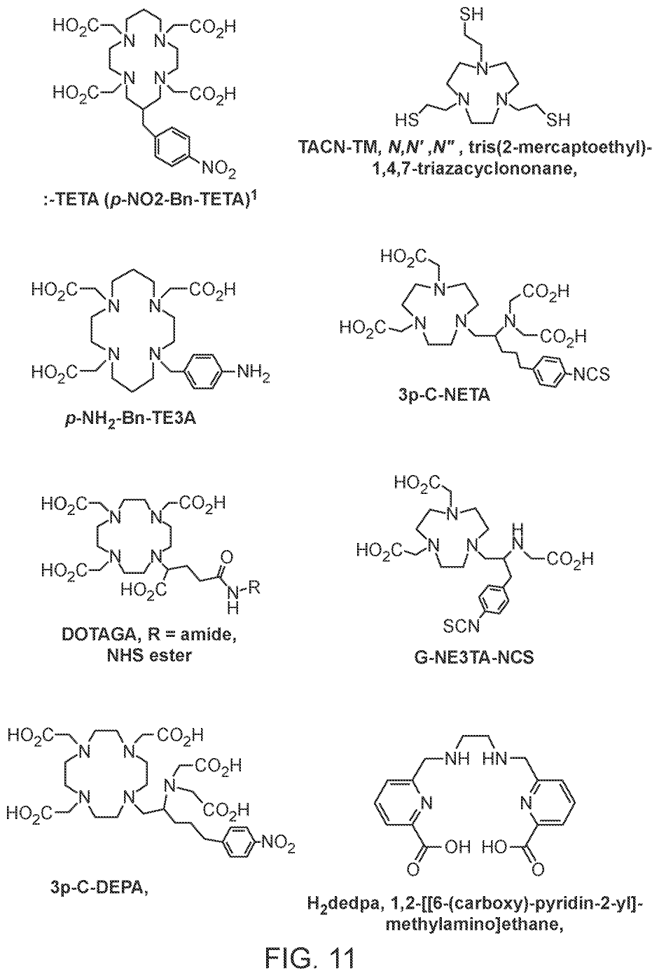

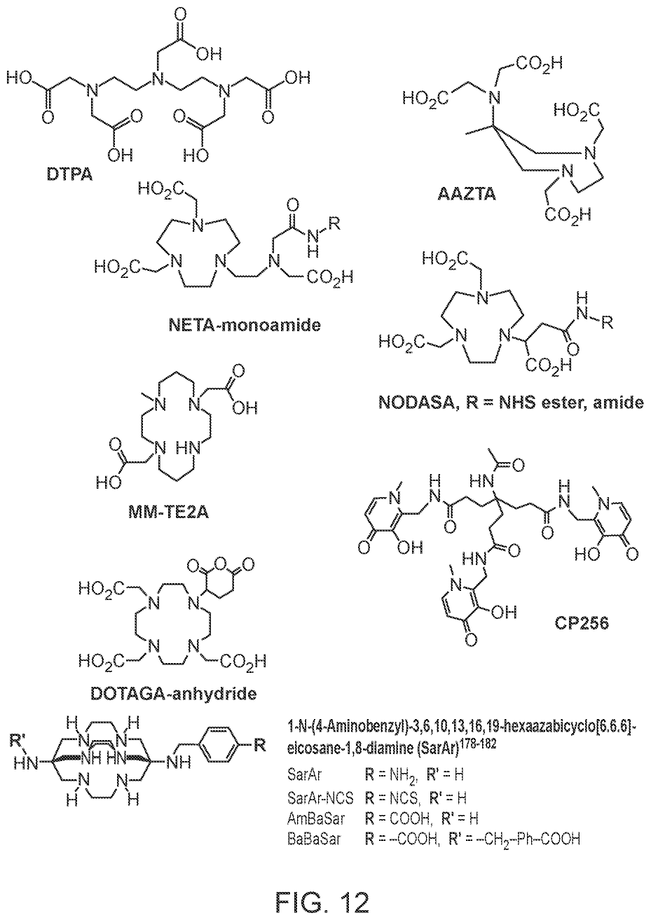

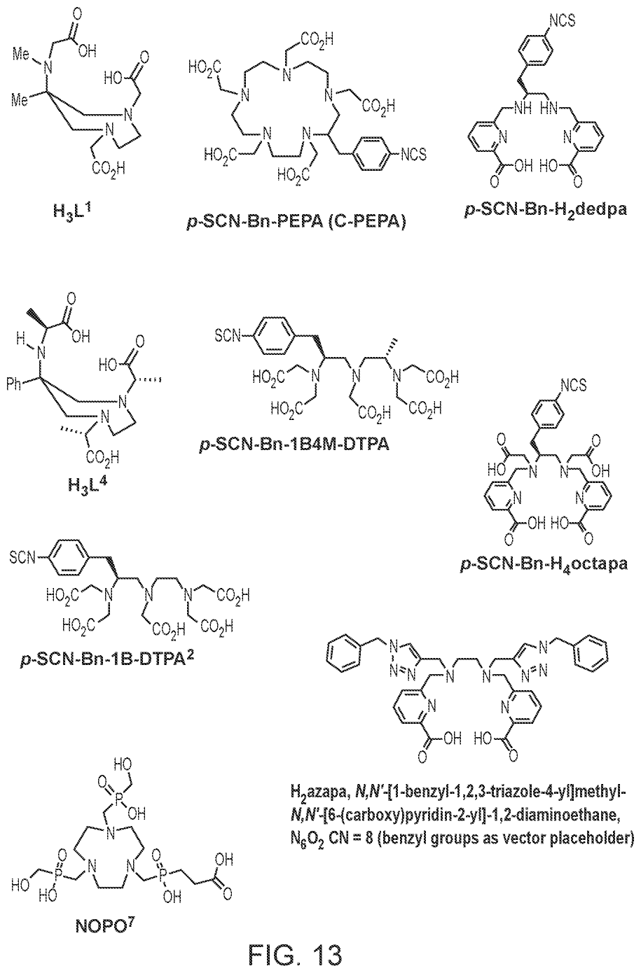

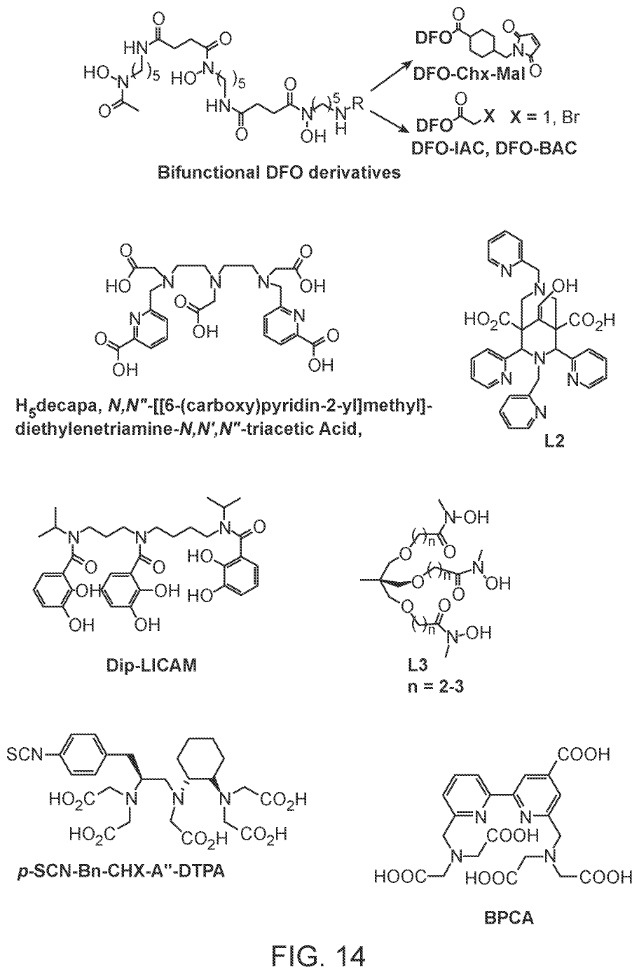

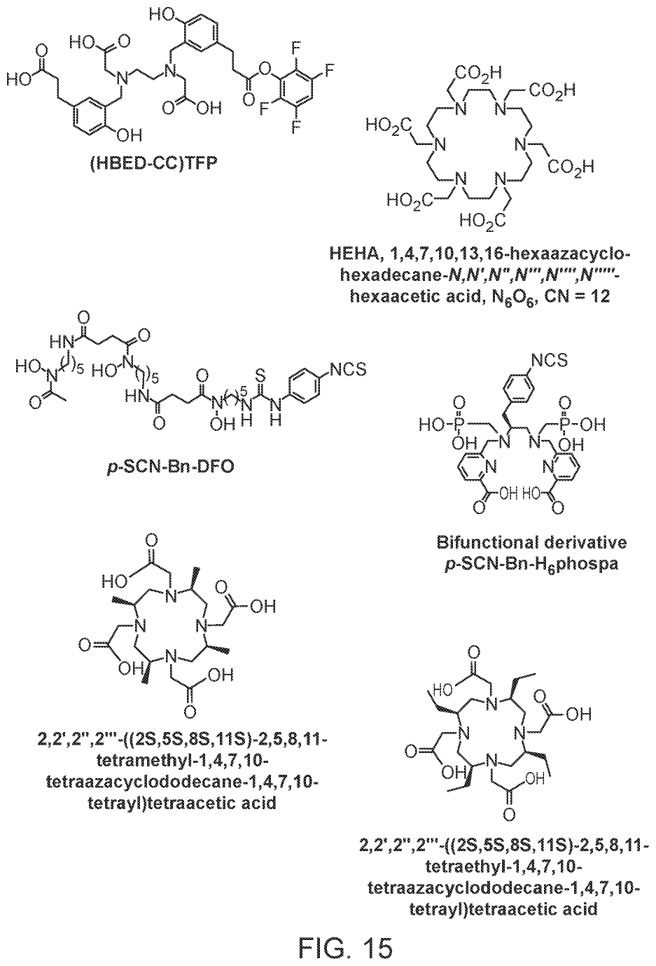

In some embodiments, a conjugate disclosed herein comprises a conjugate of Table 5A, or a salt or solvate thereof. In some embodiments, a conjugate disclosed herein is a conjugate of Table 5B, or a salt or solvate thereof. In some embodiments, a conjugate disclosed herein comprises a conjugate of Table 5C, or a salt or solvate thereof. In some embodiments, a conjugate disclosed herein is a conjugate of Table 5D, or a salt or solvate thereof. In some embodiments, a conjugate disclosed herein comprises a conjugate of Tables 5A and 5C, and a radionuclide selected from Tables 6A and 6B. In some embodiments, a conjugate disclosed herein comprises a targeting ligand selected from Table 1, a radionuclide selected from Tables 6A and 6B, a metal chelator selected from FIGS. 1-15, and optionally a linker that connects the metal chelator with the targeting ligand. In some embodiments, a conjugate disclosed herein comprises a targeting ligand selected from Table 1 and a metal chelator selected from FIGS. 1-15.

### Metal Chelator

In one aspect, described herein are conjugates (e.g., conjugates of Formula (X), Formula (X-III), and Formula (X-IV)) that comprise a metal chelator that is configured to bind with a radionuclide. The metal chelator can refer to a moiety of the conjugate that is configured to bind with a radionuclide. In some embodiments, a conjugate described herein comprises two or more independent metal chelators, e.g., 2, 3, 4, 5, or more metal chelators. In some embodiments, a conjugate described herein comprises two metal chelators, which can be the same or different. In some embodiments, a conjugate described herein comprises two or more metal chelators. In some embodiments, the conjugate comprises two radionuclides bound to the metal chelators. The metal chelator can be attached to the linker or the peptide through any suitable group/atom of the chelator.

In some embodiments of a conjugate described herein (e.g., conjugates of Formula (X), Formula (X-III), and Formula (X-IV)), the metal chelator is capable of binding a radioactive atom. The binding can be direct, e.g., the metal chelator can make hydrogen bonds or electrostatic interactions with the radioactive atom. The binding can also be indirect. e.g., the metal chelator binds to a molecule that comprises a radioactive atom. In some embodiments, the metal chelator comprises, or is, a macrocycle. In some embodiments, the metal chelator comprises, or is, 2,2′,2″,2′″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle. e.g., a macrocycle comprising an O and/or a N, DOTA. NOTA, one or more amines, one or more ethers, one or more carboxylic acids. EDTA, DTPA. TETA, DO3A. PCTA, or desferrioxamine.

In some embodiments of a conjugate described herein (e.g., conjugates of Formula (X), Formula (X-III), and Formula (X-IV)), the metal chelator comprises a plurality of amines. In some embodiments, the metal chelator includes 4 or more N, 4 or more carboxylic acid groups, or a combination thereof. In some embodiments, the metal chelator does not comprise S. In some embodiments, the metal chelator comprises a ring. In some embodiments, the ring comprises an O and/or an N. In some embodiments, the metal chelator is a ring that includes 3 or more N, 3 or more carboxylic acid groups, or a combination thereof. In some embodiments, the metal chelator is poly polydentate.

In some embodiments of a conjugate described herein (e.g., conjugates of Formula (X), Formula (X-III), and Formula (X-IV)), a metal chelator described herein is selected from: DOTA, DOTA-GA, pBn-DOTA, pBn-SCN-DOTA, NH₂-DOTA, NH₂-DOTA-GA, p-NCS-Bn-DOTA-GA, p-NH₂-Bn-oxo-DO3A, p-SCN-Bn-oxo-DO3A, NOTA, NODA-GA, NH₂-NODA-GA, p-NCS-Bn-NODA-GA, p-NH₂-Bn-NOTA, p-SCN-Bn-NOTA, NCS-MP-NODA, NH₂-MPAA-NODA, PCTA, p-NH₂-Bn-PCTA, p-SCN-Bn-PCTA, p-SCN-Bn-HEHA, H2-MACROPA-NCS, H1-MACROPA, H2-MACROPA-NH₂, H4-OCTAPA, tetra-(S, S, S, S)-Mc-DOTA, tetra-(S, S, S, S)-Et-DOTA, tetra-(S, S, S, S)-iBu-DOTA, or maleimide-nBu-DOTA.

In some embodiments of a conjugate described herein (e.g., conjugates of Formula (X), Formula (X-III), and Formula (X-IV)), a metal chelator described herein has a structure of

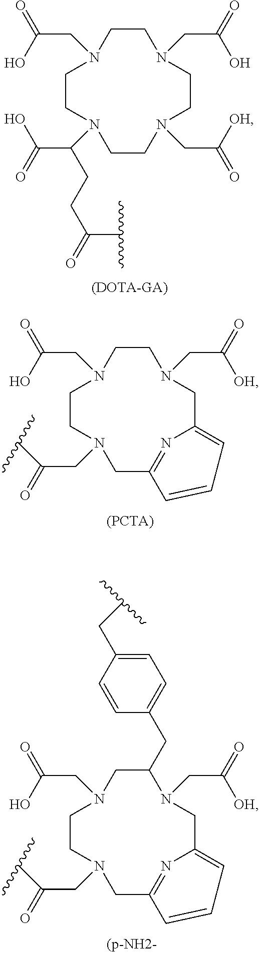 [SMILES: *C(=O)CCC(C(=O)O)N1CCN(CC(=O)O)CCN(CC(=O)O)CCN(CC(=O)O)CC1 ; *C(=O)CN1CCN(CC(=O)O)CCN(CC(=O)O)Cc2cccc(n2)C1 ; *Cc1ccc(CC2CN(CC(=O)O)CCN(CC(*)=O)Cc3cccc(n3)CN2CC(=O)O)cc1]

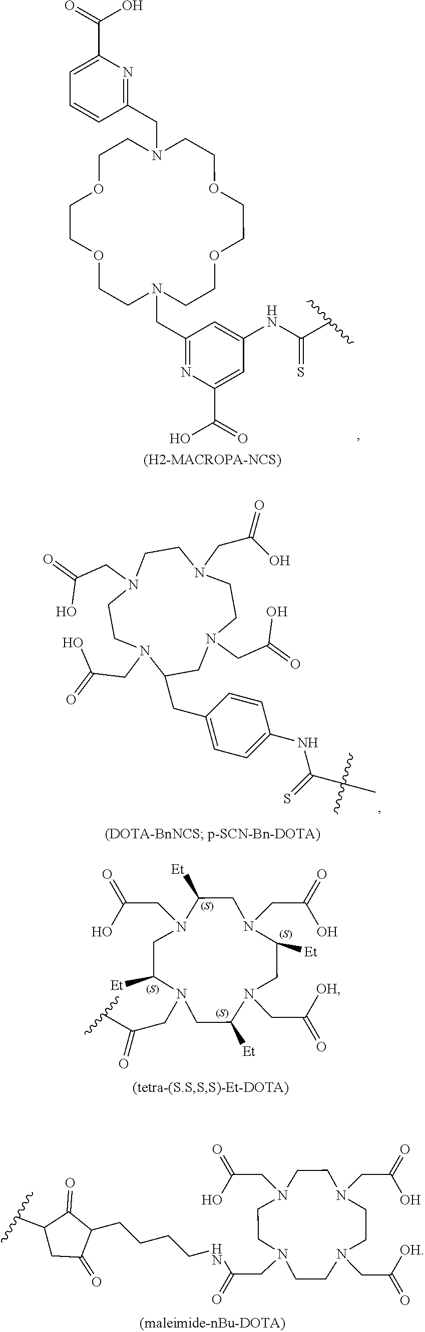

In some embodiments, a metal chelator described herein has a structure of

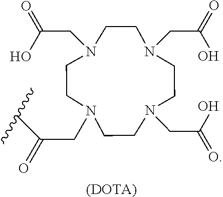

In some embodiments of a conjugate described herein (e.g., conjugates of Formula (X), Formula (X-III), and Formula (X-IV)), a metal chelator described herein comprises a cyclic chelating agent. Exemplary cyclic chelating agents include, but are not limited to, AAZTA, BAT, BAT-TM. Crown, Cyclen, DO2A. CB-DO2A, DO3A, H3HP-DO3A, Oxo-DO3A, p-NH₂-Bn-Oxo-DO3A, DOTA, DOTA-3py. DOTA-PA, DOTA-GA, DOTA-4AMP. DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂—Bn-CHX-A″-DTPA, p-SCN-DTPA, p-SCN-Bz-Mx-DTPA, IB4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H₅decapa, bispa², H₄pypa, H₄octapa, H₄CHXoctapa, p-SCN-Bn-H₄octapa, p-SCN-Bn-H₄octapa, TTHA, p-NOrBn-neunpa, H₄octox, H₂macropa, H-bispa², H₄phospa, H₆phospa, p-SCN-Bn-H₆phospa, TETA, p-NO₂-Bn-TETA, TRAP, TPA, HBED, SHBED, HBED-CC, (HBED-CC)TFP, DMSA, DMPS, DHLA, lipoic acid, TGA, BAL, Bis-thioseminarabazones, p-SCN-NOTA, nNOTA, NODAGA, CB-TE1AlP, 3P-C-NETA-NCS, 3p-C-DEPA, 3P-C-DEPA-NCS, TCMC, PCTA, NODIA-Me, TACN, pycup1A1B, pycup2A, THP, DEDPA, H₂DEDPA, p-SCN-Bn-H₂DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, L^{py}, L¹, L², L³, and EuK-106, In some embodiments, the metal chelator is DOTA, TRITA, TETA, DOTA-MA, DO3A-HP, DOTMA, DOTA-pNB, DOTP, DOTMP, DOTEP, DOTMPE, F-DOTPME, DOTPP, DOTBzP, DOTA-monoamide, p-NCS-ROTA, p-NCS-PADOTA, BAT, DO3TMP-Monoamide, p-NCS-TRITA, NOTA, and CHX-A″-DTPA. In some embodiments, a metal chelator described herein comprises an acyclic chelating agent. Exemplary acyclic chelating agents include, but are not limited to, DTA, CyEDTA, EDTMP, DTPMP, DTPA, CyDTPA, Cy2DTPA, DTPA-MA, DTPA-BA, and BOPA. In some embodiments, a metal chelator described herein comprises DOTA, DOTP. DOTMA. DOTAM. DTPA, NTA, EDTA, DO3A. DO2A, NOC, NOTA, TETA. TACN. DiAmSar, CB-Cyclam, CB-TE2A. DOTA-4AMP, or NOTP. In some embodiments, a metal chelator described herein comprises H₄pypa, H₄octox, H₄octapa, p-NO₂-Bn-neunpa, p-SCN-Bn-H₄neunpa, TTHA, ^{t}Bu₄pypa-C₇-NHS, H₄neunpa, Hzmacropa, HP-DO3A, BT-DO3A, DO3A-Nprop, DO3AP, DO2A2P, DOA3P, DOTP, DOTPMB, DOTAMAE, DOTAMAP, DO3AM, DOTMA, TCE-ROTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, or HBED.

In some embodiments of a conjugate described herein (e.g., conjugates of Formula (X), Formula (X-III), and Formula (X-IV)), the metal chelator is DO3A. In some embodiments, the metal chelator is PEPA. In some embodiments, the metal chelator is EDTA. In some embodiments, the metal chelator is CHX-A″-DTPA. In some embodiments, the metal chelator is HEHA. In some embodiments, the metal chelator is DOTMP. In some embodiments, the metal chelator is t-Bu-calix[4]arene-tetracarboxylic acid.

In some embodiments, the metal chelator is macropa. In some embodiments, the metal chelator is macropa-NCS. In some embodiments, the metal chelator is H₄pypa. In some embodiments, the metal chelator is H₄octapa. In some embodiments, the metal chelator is H₄CHXoctapa. In some embodiments, the metal chelator is DOTP. In some embodiments, the metal chelator is crown.

In some embodiments of a conjugate described herein (e.g., conjugates of Formula (X), Formula (X-III), and Formula (X-IV)), the metal chelator is DOTA. In some embodiments, the metal chelator is a chiral derivative of DOTA. Exemplary chiral DOTA chelators are described in Dai et al., Nature Communications (2018) 9:857. In some embodiments, the metal chelator is 2,2′,2″,2′″-((2S,5S, 8S, 11S)-2,5,8,11-tetramethyl-1,4,7,10-tetraazacyclododecane-1,4,7,10-tetrayl)tetraacetic acid. In some embodiments, the metal chelator has a structure of

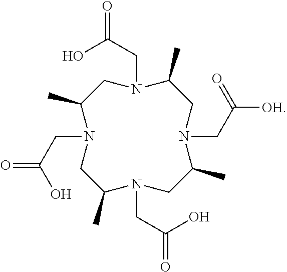 [SMILES: C[C@H]1CN(CC(=O)O)[C@@H](C)CN(CC(=O)O)[C@@H](C)CN(CC(=O)O)[C@@H](C)CN1CC(=O)O]

In some embodiments, the metal chelator is 2,2′,2″,2′″-((2S,5S, 8S, 11S)-2,5,8,11-tetraethyl-1,4,7,10-tetraazacyclododecane-1,4,7,10-tetrayl)tetraacetic acid. In some embodiments, the metal chelator has a structure of

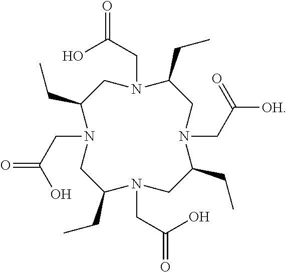 [SMILES: CC[C@H]1CN(CC(=O)O)[C@@H](CC)CN(CC(=O)O)[C@@H](CC)CN(CC(=O)O)[C@@H](CC)CN1CC(=O)O]

In some embodiments of a conjugate described herein (e.g., conjugates of Formula (X), Formula (X-III), and Formula (X-IV)), the metal chelator has a structure of

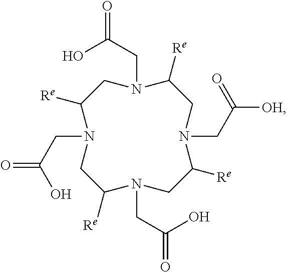

wherein each R^{c} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments, the metal chelator has a structure of

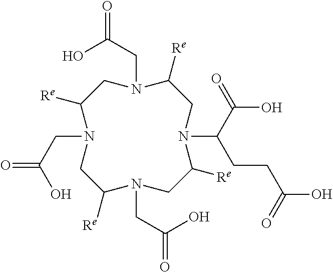

wherein each R^{c} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain.

In some embodiments, the conjugate comprises DOTA. In some embodiments, the conjugate comprises a DOTA derivative such as p-SCN-Bn-DOTA and MeO-DOTA-NCS. In some embodiments, the conjugate comprises two independent metal chelators, and at least one or both are DOTA. The structures of some exemplary metal chelators are illustrated in FIGS. 1-15 (without showing the attachment points). Exemplary metal chelators are further described in WO2012/174136: US20130183235A1; US20120219495A1; Ramogidaand et al., EJNMMI radiopharm, chem. 4, 21 (2019): Thiele et al., Cancer Biotherapy and Radiopharmaceuticals 2018; Li et al., Bioconjugate Chem. 2019, 30, 5, 1539-1553; and Baranyai et al., Eur. J. Inorg. Chem. 36-56 (2020), each of which is incorporated by reference in its entirety.

### Radionuclide

In one aspect, described herein are conjugates (e.g., a conjugate of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), Formula (IVe), Formula (X), Formula (X-III), or Formula (X-IV)) that comprise a radionuclide. Exemplary radionuclides include, but are not limited to, astatine-211, astatine-217, actinium-225, americium-243, radium-223, lead-212, lead-203, copper-64, copper-67, copper-60, copper-61, copper-62, bismuth-212, bismuth-213, gallium-68, gallium-67, dysprosium-154, gadolinium-148, gadolinium-153, samarium-146, samaritan-147, samarium-153, terbium-149, thorium-227, thoritun-229, iron-59, yttrium-86, indium-111, holmium-166, technetium-94, technetium-99^{m}, yttrium-90, lutetium-177, terbium-161, rhenium-186, rhenium-188, cobalt-55, scandium-43, scandium-44, scandium-47, dysprosium-166, fluorine-18, or iodine-131.

Generally, the type of radionuclide used in a therapeutic radiopharmaceutical can be tailored to the specific type of cancer, the type of targeting moiety, etc. Radionuclides that undergo α-decay produce particles composed of two neutrons and two protons, and radionuclides that undergo β-decay emit energetic electrons from their nuclei. Radionuclides that undergo β+-decay emit positrons which can be detected with positron emission tomography (PET). Substitution with positron emitting isotopes, such as carbon-11, nitrogen-13, oxygen-15, and fluorine-18, can be useful in PET imaging studies. Some radionuclides can also emit Auger. In some embodiments, the conjugate comprises an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical conjugate. Tables 6A, 6B, 6C, and 6D below illustrate some properties of exemplary radionuclides.

In one aspect, described herein are conjugates that comprise a radionuclide. Generally, the type of radionuclide used in a therapeutic radiopharmaceutical can be tailored to the specific type of cancer, the type of targeting moiety (e.g., KRAS G12C covalent binders), etc. Radionuclides that undergo α-decay produce particles composed of two neutrons and two protons, and radionuclides that undergo β-decay emit energetic electrons from their nuclei. Some radionuclides can also emit Auger. In some embodiments, the conjugate comprises an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical conjugate. Tables 6A, 6B, 6C, and 6D below illustrate some properties of exemplary radionuclides.

TABLE 6A
Exemplary radionuclides suitable
for therapeutic use with a metal chelator

| Nuclide | Emission | Half-life (days) |
|---|---|---|
| Copper-67 | β | about 2.6 |
| Rhodium-105 | β | about 1.5 |
| Indium-111 | β | about 2.8 |
| Actinium-225 | α | about 10.0 |
| (Ac-225) | | |
| Lutetium | β | about 6.7 |
| (Lu-177) | | |
| Radium-223 | α | about 11.4 |
| Radium-224 | α | about 3.63 |
| Astatine-211 | α | about 0.3 |
| Yttrium-90 | β | about 2.7 |
| Gadolinium-148 | α | about 74 |
| Samarium-153 | β | about 1.9 |
| Promethium-153 | β | about 5.25 min. |
| Thulium-167 | β | about 9.3 |
| Ytterbium-175 | β | about 4.2 |
| Lead-212 | β | about 0.4 |
| Bismuth-209 | α | stable |
| Bismuth-212 | α | about 0.04 |
| Bismuth-213 | α | about 0.03 |
| Polonium-213 | α | about 4.2 μ sec. |
| Francium-223 | α | about 0.015 |
| Thorium-227 | α | about 18.7 |
| Thorium-229 | α | about 7.3 |
| Terbium-149 | α | about 0.17 |

TABLE 6B
Exemplary radionuclides suitable
for diagnostic use with a metal chelator

| Nuclide | Half-life |
|---|---|
| Lutetium-177 (Lu-177) | about 6.7 days |
| Indium-111 (In-111) | about 2.8 days |
| Gallium-68 (Ga-68) | about 68 minutes |
| Copper-62 (Cu-62) | about 9.6 minutes |
| Copper-64 (Cu-64) | about 12.7 hours |
| Zirconium-89 (Zr-89) | about 78.4 hours |
| Technetium-99m | about 6 hours |
| Cerium-134 (Ce-134) | about 3.2 days |
| Terbium-152 | about 17.5 hours |

TABLE 6C
Exemplary covalent radionuclides
suitable for therapeutic use

| | | Half-life |
|---|---|---|
| Nuclide | Emission | (days) |
| Astatine-211 | α | about 0.3 |
| Iodine-131 | β | about 8 |

TABLE 6D
Exemplary covalent radionuclides
suitable for diagnostic use

| Nuclide | Half-life |
|---|---|
| Iodine-125 (1-125) | about 60 days |
| Iodine-124 (1-124) | about 4.2 days |
| Iodine-123 (1-123) | about 13 hours |
| Fluorine-18 (F-18) | about 110 minutes |
| Carbon-11 (C-11) | about 20 minutes |
| Nitrogen-13 (N-13) | about 10 minutes |
| Bromine-76 (Br-76) | about 16.2 hours |
| Oxygen-15 (O-15) | about 122 seconds |
| Arsenic-74 (As-74) | about 17.8 days |

In some embodiments, a conjugate described herein comprises one or more independent radionuclides. In some embodiments, the conjugate comprises two radionuclides. In some embodiments, each of the one or more radionuclides is bound to a metal chelator of the conjugate. In some embodiments, two radionuclides of a conjugate are bound to the same metal chelator. In some embodiments, two radionuclides of a conjugate are bound to two independent metal chelators. In some embodiments, each of the one or more radionuclides is an alpha particle-emitting radionuclide.

In some embodiments, a conjugate described herein comprises an alpha particle-emitting radionuclide. In some embodiments, the alpha particle-emitting radionuclide is actinium-225 (²²⁵Ac), radium-223 (²²³Ra), radium-224 (²²⁴Ra), bismuth-209 (²⁰⁹Bi), bismuth-213 (²¹³Bi), Gadolinium-148 (¹⁴⁸Gd), Terbium-149 (¹⁴⁹Tb), polonium-213 (²¹³Po), francium-223 (²²³Fr), thorium-227 (²²⁷Th), or thorium-229 (²²⁹Th). In some embodiments, the alpha particle-emitting radionuclide is selected from ¹⁴⁸Gd, ¹⁴⁹Tb, ²⁰⁹Bi, ²¹³Po, ²¹³Bi, ²²³Ra, ²²³Fr, ²²⁷Th, ²²⁵Ac, and ²²⁹Th. In some embodiments, the alpha particle-emitting radionuclide is ²²⁵Ac. In some embodiments, the alpha particle-emitting radionuclide is ²¹³Bi. In some embodiments, the alpha particle-emitting radionuclide is ²¹²Bi. In some embodiments, the alpha particle-emitting radionuclide is ²¹²Pb. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²³Th. In some embodiments, the alpha particle-emitting radionuclide is ¹⁴⁹Th. In some embodiments, the radionuclide is Zirconium-89 (⁸⁹Zr).

In some embodiments, a conjugate described herein comprises a radionuclide selected from ⁶²Cu, ⁶⁴Cu, ⁶⁷Cu, ⁹⁹Y, ¹⁰⁹Pd, ¹¹¹Ag, ¹³⁴Ce, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ¹⁵³Pm, ⁴⁴Sc, ⁴⁷Sc, ²¹³Po, ²¹²Pb, ²¹³Bi, ²¹²Bi, ²¹³Bi, ²²⁵Ac, ^{117m}Sn, ⁶⁷Ga, ¹⁴⁹Tb, ¹⁵²Tb, ¹⁶⁷Tm, ¹⁷⁵Yb, ²²³Ra, ²²³Fr, ²²⁷Th, ²⁰¹Tl, ¹⁴⁸Gd, ¹⁶⁰Gd, ¹⁴⁸Nd, ⁸⁹Sr, and ⁸⁹Zr. In some embodiments, the radionuclide is selected from ⁶²Cu, ⁶⁴Cu, ⁶⁷Cu, ⁶⁸Ga, ⁸⁹Zr, ⁹⁰Y, ^{99m}Tc, ¹⁰⁵Rh, ¹¹¹In, ¹³⁴Ce, ¹⁴⁸Gd, ¹⁴⁹Tb, ¹⁵²Tb, ¹⁵³Pm, ¹⁶⁷Tm, ¹⁷⁵Yb, ¹⁷⁷Lu, ²⁰⁹Bi, ²¹²Pb, ²¹³Po, ²¹³Bi, ²²³Fr, ²²⁷Th, ²²⁵Ac, and ²²⁹Th. In some embodiments, the radionuclide is ²²⁵Ac. In some embodiments, the radionuclide is a decay daughter of ²²⁵Ac such as ²²¹ Fr, ²¹⁷At, ²¹³Bi, ²¹³Po, ²⁰⁹Tl, ²⁰⁹Pb, or ²⁰⁹Bi. In some embodiments, the conjugate comprises two ²²⁵Ac radionuclides. In some embodiments, the radionuclide is ¹⁷⁷Lu. In some embodiments, the conjugate comprises two ¹⁷⁷Lu radionuclides.

In some embodiments, the conjugate comprises an alpha particle-emitting radionuclide bound to the metal chelator. In some embodiments, the alpha particle-emitting radionuclide is actinium-225, thorium-227, or radium-223. In some embodiments, the alpha particle-emitting radionuclide is actinium-225, bismuth-213, bismuth-209, terbium-149, radium-223, thoritun-227, francium-223, gadolinium-148, thorium-229 or polonium-213. In some embodiments, the alpha particle-emitting radionuclide is actinium-225.

In some embodiments, the conjugate comprises a beta particle-emitting radionuclide bound to the metal chelator. In some embodiments, the beta particle emitting radionuclide is zirconium-89, yttrium-90, samaritan-153, lutetium-177, or lead-212.

In some embodiments, the radionuclide is an alpha particle-emitting radionuclide. In some embodiments, the alpha particle-emitting radionuclide is selected from actinium-225, radium-223, lead-204, and thorium-227. In some embodiments, the radionuclide is a beta particle-emitting radionuclide. In some embodiments, the beta particle-emitting radionuclide is lutetium-177, copper-64, zircronium-89, yttrium-90, copper-67, indium-111, samarium-153, rhodium-105, ytterbium-175, thulium-167 or lead-212. In some embodiments, the beta particle-emitting radionuclide is lutetium-177. In some embodiments, the radionuclide is a gamma particle-emitting radionuclide. In some embodiments, the gamma particle-emitting radionuclide is indium-111 or tin-117m. In some embodiments, the radionuclide is a positron-emitting radionuclide. In some embodiments, the positron-emitting radionuclide is gallium-68, copper-64, or yttrium-90. In some embodiments, the conjugate comprises a gamma particle emitting radionuclide. In some embodiments, the gamma particle emitting radionuclide is indium-111.

In some embodiments, conjugates described herein do not contain any radionuclide, i.e., a cold conjugate. For example, in some cases, a radionuclide can be replaced with a surrogate (e.g., ²²⁵Ac replaced with lanthanum) for testing and experimental purposes.

In some embodiments, the radionuclide is no-carrier added (i.e., non-carrier-added or n.c.a.) ¹⁷⁷Lu. In some embodiments, the radionuclide is no-carrier added (i.e., non-carrier-added or n.c.a.)²²⁵Ac. In some embodiments, the radionuclide is ¹⁷⁷Lu free of long-lived radioactive contaminants and byproducts. In some embodiments, the radionuclide is a non-carrier-added radionuclide.

In some embodiments, a compound or protein described herein comprises one or more independent radionuclides. In some embodiments, the compound or protein comprises two radionuclides. In some embodiments, each of the one or more radionuclides is an alpha particle-emitting radionuclide. In some embodiments, each of the one or more radionuclides is a beta particle-emitting radionuclide. In some embodiments, a radiolabeled compound described herein comprises a radionuclide selected from ¹¹C, ¹³N, ¹⁵O, ¹⁸F, ²⁰As, ²¹As, ⁷²As, ⁷³As, ⁷⁴As, ⁷⁶As, ⁷⁷As, ⁷⁶Br, ¹²³I, ¹²⁴I, ¹²⁵I. ¹³¹I, and ²¹¹At.

In some embodiments, a radiolabeled compound described herein comprises an alpha panicle-emitting radionuclide. In some embodiments, the alpha particle-emitting radionuclide is astatine-211 (²¹¹At).

In some embodiments, the compound or protein comprises a covalently bound beta particle-emitting radionuclide. In some embodiments, the beta particle emitting radionuclide is iodine-131.

In some embodiments, the compound or protein comprises a covalently bound β+ positron-emitting radionuclide. In some embodiments, the β+ positron emitting radionuclide is fluorine-18.

In some embodiments, the compound or protein comprises a gamma particle emitting radionuclide. In some embodiments, the gamma particle emitting radionuclide is iodine-123.

In some embodiments, provided herein are compounds having the structures of the radiolabeled compounds described herein, except that the radioisotope is replaced with a surrogate (e.g., ¹³¹I replaced with iodine), i.e., a cold compound. In some embodiments, a radionuclide of the radiolabeled compounds described herein can be replaced with a surrogate (e.g., ¹³¹I replaced with iodine) for testing and experimental purposes.

In some embodiments, a radiolabeled compound (i.e., a radiopharmaceutical conjugate) described herein is designed to have a prescribed elimination profile. The elimination profile can be designed by adjusting the chemical properties of the radiolabeled compound, the chemical properties of the linker, etc. In some embodiments, the radiolabeled compound has an elimination half-life in mammals of about 0.1 to about 120 hours. In some embodiments, the radiolabeled compound has an elimination half-life in mammals of about 10 minutes to 30 minutes. In some embodiments, the radiolabeled compound has an elimination half-life in mammals of about 30 minutes to 60 minutes. In some embodiments, the radiolabeled compound has an elimination half-life in mammals of about 1 hour to 2 hours. In some embodiments, the radiolabeled compound has an elimination half-life in mammals of about 2 hours to 3 hours. In some embodiments, the radiolabeled compound has an elimination half-life in mammals of about 3 hours to 4 hours. In some embodiments, the radiolabeled compound has an elimination half-life in mammals of about 4 hours to 5 hours. In some embodiments, the radiolabeled compound has an elimination half-life in mammals of about 5 hours to 6 hours. In some embodiments, the radiolabeled compound has an elimination half-life of at least 15 minutes, 30 minutes, 1 hour, 2 hours, 3 hours, 4 hours, 5 hours, 7 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, the radiolabeled compound has an elimination half-life of at most 120 hour, 80 hours, 70 hours, 60 hours, 50 hours, 40 hours, 30 hours, 24 hours, 12 hours, 10 hours, 5 hours, 3 hours, 2 hours, 1 hour, 30 minutes, or 15 minutes. In some embodiments, the radiolabeled compound has an elimination half-life of about 0.1 to 24 hours. In some embodiments, the radiolabeled compound has an elimination half-life of about 10 minutes to 1 hour. In some embodiments, the radiolabeled compound has an elimination half-life of about 30 minutes to 12 hours. In some embodiments, the radiolabeled compound has an elimination half-life of about 2 to 24 hours. In some embodiments, the radiolabeled compound has an elimination half-life of about 6 to 24 hours. In some embodiments, the elimination half-life is determined in mice. In some embodiments, the elimination half-life is determined in rats. In some embodiments, the elimination half-life is determined in humans.

In some embodiments, a radiolabeled compound described herein can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the radiolabeled compound in a tumor is at least about 0.1, 0.5, 1, 3, 6, 12, 24, 48, 72, 96 or more than 96 hours. In some embodiments, the elimination half-life of the radiolabeled compound in a tumor tissue is at least 1.1, 1.2, 1.3, 1.4, 1.5, 2.0, 2.5, 3.0, 4.0, 5.0, 10, 25, 50, or 100 fold greater than the elimination half-life of the a radiolabeled compound in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the cold pharmaceutical in the living system. In some embodiments, the elimination half-life is measured as effective half-life, which is the half-life of a radiopharmaceutical in a living system taking into account the half-life of the radioisotope.

A radiolabeled compound described herein can have a described time-integrated activity coefficient (i.e., a) in a tumor or non-tumor tissues of a subject. As used herein, A represents the cumulative number of nuclear transformations occurring in a source tissue over a dose-integration period per unit administered activity. The ã value of a radiolabeled compound can be tuned by modifications of the radiolabeled compound. The A value can be determined using a method known in the art. In some embodiments, the ã value of the radiolabeled compound in a tumor is from about 6 hours to 14 days. In some embodiments, the ã value in a tumor is about 2 to 10 days. In some embodiments, the ã value in a tumor is about 4 to 7 days. In some embodiments, the ã value in a tumor is about 7 to 10 days. In some embodiments, the 3 value in a tumor is from about 1 day, 2 days, 3 days, or 4 days to about 5 days, 6 days, 7 days, 8 days, 9 days, 10 days, 11 days, or 12 days. In some embodiments, the ã value in a tumor is about 3 days, 4 days, 5 days, 6 days, 7 days, 8 days, 9 days, 10 days, 11 days, or 12 days. In some embodiments, the ã value of the radiolabeled compound in a non-tumor tissue is from about 6 hours to 14 days. In some embodiments, the 3 value in a non-tumor tissue is about 2 to 10 days. In some embodiments, the ã value in a non-tumor tissue is about 4 to 7 days. In some embodiments, the ã value in a non-tumor tissue is about 7 to 10 days. In some embodiments, the ã value in a non-tumor tissue is from about 1 day, 2 days, 3 days, or 4 days to about 5 days, 6 days, 7 days, 8 days, 9 days, 10 days, 11 days, or 12 days. In some embodiments, the ã value in a non-tumor tissue is about 3 days, 4 days, 5 days, 6 days, 7 days, 8 days, 9 days, 10 days, 11 days, or 12 days. The ã value of the radiolabeled compound in a tumor can be the same as the ã value of the radiolabeled compound in a non-tumor tissue of the subject. The ã value of the radiolabeled compound in a tumor can be longer or shorter than the ã value of the radiolabeled compound in a non-tumor tissue of the subject. In some embodiments, the ã value of the radiolabeled compound in a tumor is at least 1.1, 1.2, 1.3, 1.4, 1.5, 2.0, 2.5, 3.0, 4.0, or 5.0 fold of the 5 value of the radiolabeled compound in a non-tumor tissue of the subject.

A radiolabeled compound described herein can have an ã value in an organ of a subject. In some embodiments, the radiolabeled compound has an ã value in a kidney of the subject of at most 24 hours. In some embodiments, the ã value of the radiolabeled compound in a kidney of the subject is at most 18 hours, hours, 12 hours, 10 hours, 8 hours, 6 hours, or 5 hours. In some embodiments, the ã value of the radiolabeled compound in a kidney of the subject is about 30 minutes to about 24 hours. In some embodiments, the ã value of the radiolabeled compound in a kidney of the subject is about 2 to 24 hours.

In some embodiments, the ã value of the radiolabeled compound in a kidney of the subject is more than 24 hours. In some embodiments, the ã value of the radiolabeled compound in a liver of the subject is at most 24 hours. In some embodiments, the ã value of the radiolabeled compound in a liver of the subject is at most 18 hours, 15 hours, 12 hours, 10 hours, 8 hours, 6 hours, or 5 hours. In some embodiments, the a value of the radiolabeled compound in a liver of the subject is about 30 minutes to about 24 hours. In some embodiments, the ã value of the radiolabeled compound in a liver of the subject is about 2 to 24 hours. In some embodiments, the ã value of the radiolabeled compound in a liver of the subject is more than 24 hours.

In some cases, the elimination profile of the radiolabeled compound can be adjusted by a reversible binding between the radiolabeled compound and a plasma protein such as albumin. A suitable affinity between the radiolabeled compound and the plasma protein can utilize the plasma protein as a reservoir for the radiolabeled compounds, attaching and preserving the radiolabeled compound at high concentration and releasing the radiolabeled compound at a lower concentration, thereby improving elimination profile. In some embodiments, a dissociation constant (Kd) between the radiolabeled compound and human serum albumin is at most 500 μM, as determined at room temperature in human serum condition. In some embodiments, the Kd is from about 0.1 nM to about 1000 μM. In some embodiments, the Kd is at most 100 μM. In some embodiments, the Kd is at most 15 μM. In some embodiments, the Kd is from about 1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

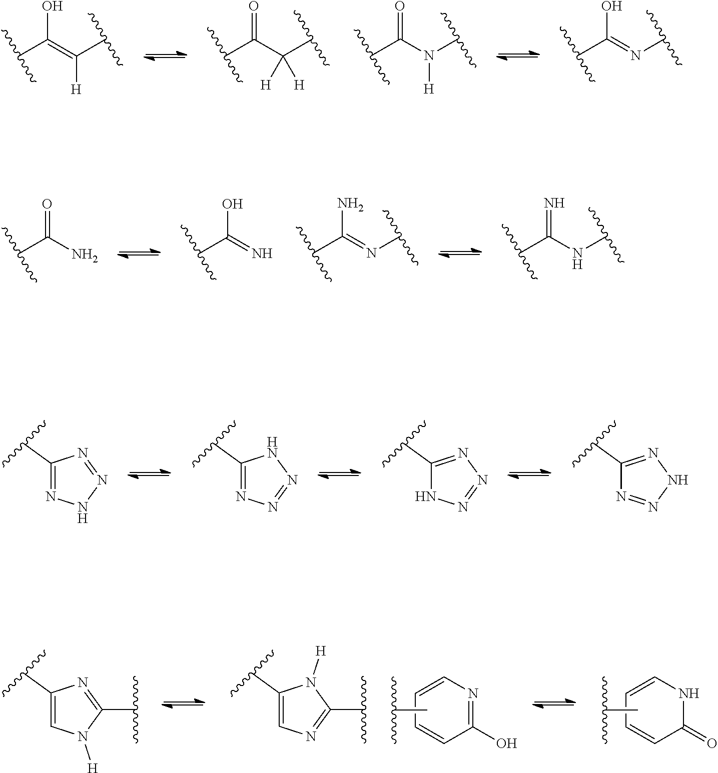

In some instances, the compounds disclosed herein exist in tautomeric forms. The structures of said compounds are illustrated in the one tautomeric form for clarity. The alternative tautomeric forms are expressly included in this disclosure.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically -labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively, Compounds described herein, and the pharmaceutically acceptable salts, solvates, or stereoisomers thereof which contain the aforementioned isotopes and/or other isotopes of other atoms are within the scope of this disclosure. Certain isotopically -labeled compounds, for example those into which radioactive isotopes such as ³H and ¹⁴C are incorporated, are useful in drug and/or substrate tissue distribution assays. Tritiated, i.e., ³H and carbon-14. i.e., ¹⁴C, isotopes are notable for their ease of preparation and detectability. Further, substitution with heavy isotopes such as deuterium, i.e., 2H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions. As used herein, a “pharmaceutically acceptable salt” refers to any salt of a compound that is useful for therapeutic purposes of a subject.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N+(C₁₋₄ alkyl)4, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA). TGA-mass spectroscopy, TGA-Infrared spectroscopy, powder X-ray diffraction (PXRD), Karl Fisher titration, high resolution X-ray diffraction, and the like.

### Preparation of the Compound

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh. PA), Aldrich Chemical (Milwaukee, WI, including Sigma Chemical and Fluka). Apin Chemicals Ltd. (Milton Park, UK). Avocado Research (Lancashire. U.K.), BDH, Inc. (Toronto, Canada). Bionet (Cornwall. U.K.), Chem Service Inc. (West Chester, PA), Crescent Chemical Co. (Hauppauge. NY), Eastman Organic Chemicals, Eastman Kodak Company (Rochester, NY), Fisher Scientific Co. (Pittsburgh, PA). Fisons Chemicals (Leicestershire, UK). Frontier Scientific (Logan. UT), ICN Biomedicals, Inc. (Costa Mesa. CA), Key Organics (Cornwall, U.K.). Lancaster Synthesis (Windham, NH), Maybridge Chemical Co. Ltd. (Cornwall, U.K.), Parish Chemical Co. (Orem. UT), Pfaltz & Bauer, Inc. (Waterbury, CN), Polyorganix (Houston. TX). Pierce Chemical Co. (Rockford, IL), Riedel de Haen AG (Hanover. Germany). Spectrum Quality Product. Inc. (New Brunswick. NJ). TCI America (Portland, OR), Trans World Chemicals, Inc. (Rockville, MD), and Wako Chemicals USA, Inc. (Richmond. VA).

Suitable reference books and treatises that detail the synthesis of reactants useful in the preparation of compounds described herein, or provide references to articles that describe the preparation, include for example. “Synthetic Organic Chemistry”, John Wiley & Sons, Inc., New York; S. R. Sandler et al., “Organic Functional Group Preparations.” 2nd Ed., Academic Press, New York, 1983; H. O. House, “Modern Synthetic Reactions”, 2nd Ed., W. A. Benjamin. Inc. Menlo Park, Calif. 1972; T. L. Gilchrist. “Heterocyclic Chemistry”, 2nd Ed., John Wiley & Sons, New York, 1992: J. March, “Advanced Organic Chemistry: Reactions, Mechanisms and Structure”, 4th Ed., Wiley-Interscience, New York, 1992.

Additional suitable reference books and treatises that detail the synthesis of reactants useful in the preparation of compounds described herein, or provide references to articles that describe the preparation, include for example, Fuhrhop. J, and Penzlin G. “Organic Synthesis: Concepts, Methods, Starting Materials”, Second. Revised and Enlarged Edition (1994) John Wiley & Sons ISBN: 3-527-29074-5; Hoffman, R. V. “Organic Chemistry. An Intermediate Text” (1996) Oxford University Press, ISBN 0-19-509618-5: Larock, R. C. “Comprehensive Organic Transformations: A Guide to Functional Group Preparations” 2nd Edition (1999) Wiley-VCH, ISBN: 0-471-19031-4: March. J. “Advanced Organic Chemistry: Reactions, Mechanisms, and Structure” 4th Edition (1992) John Wiley & Sons. ISBN: 0-471-60180-2; Otera. J. (editor) “Modern Carbonyl Chemistry” (2000) Wiley-VCH. ISBN: 3-527-29871-1; Patai, S. “Patai's 1992 Guide to the Chemistry of Functional Groups” (1992) Interscience ISBN: 0-471-93022-9: Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta. Zurich, 2002.

### IV. Pharmaceutical Compositions

The radiopharmaceutical conjugate described herein, including e.g., pharmaceutically acceptable salt or solvate thereof, can be administered per se as a pure chemical or as a component of a pharmaceutically acceptable formulation. In some embodiments, a conjugate described herein is combined with a pharmaceutically suitable or acceptable carrier selected on the basis of a chosen route of administration and standard pharmaceutical practice as described, for example, in Remington: The Science and Practice of Pharmacy (Gennaro, 21st Ed. Mack Pub. Co., Easton, PA (2005)). Provided herein is a pharmaceutical composition comprising at least one conjugate described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a pharmaceutical composition comprising a herein described conjugate, or a pharmaceutically acceptable salt or solvate thereof, and a pharmaceutically acceptable excipient or carrier. In certain embodiments, the conjugate as described is substantially pure, in that it contains less than about 10%, less than about 5%, or less than about 1%, or less than about 0.1%, of other organic small molecules, such as unreacted intermediates or synthesis by-products that are created, for example, in one or more of the steps of a synthesis method.

Pharmaceutical compositions can include pharmaceutically acceptable carriers, diluents or excipients. Exemplary pharmaceutically acceptable carriers include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary components (e.g., preservatives, antibacterial, antiviral and antifungal agents) can also be incorporated into the compositions. Pharmaceutical compositions can be formulated to be compatible with a particular local or systemic route of administration. Thus, pharmaceutical compositions include carriers, diluents, or excipients suitable for administration by particular routes.

The compounds and pharmaceutical compositions of the current disclosure can be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. The teen parenteral as used herein includes e.g., subcutaneous, intravenous, intramuscular, intrasternal, intraperitoneal, and infusion techniques. The term parenteral also includes injections, into the eye or ocular, intravitreal, intrabuccal, transdermal, intranasal, into the brain, including intracranial and intradural, into the joints, including ankles, knees, hips, shoulders, elbows, wrists, and the like, and in suppository form. In certain embodiments, the compounds and/or formulations are administered orally. In certain embodiments, the compounds and/or formulations are administered by systemic administration. In certain embodiments, the compounds and/or formulations are administered parenterally. In certain embodiments, the compounds and/or formulations are administered locally at a targeted site.

In some embodiments, conjugates, or pharmaceutically acceptable salts or solvates thereof, and pharmaceutical compositions described herein are administered via parenteral injection as liquid solution, which can include other chemical components, such as carriers, stabilizers, diluents, dispersing agents, suspending agents, thickening agents, preservatives, or excipients. Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water soluble form. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens: antioxidants such as ascorbic acid, gentisic acid, or sodium bisulfite: chelating agents such as ethylenediaminetetraacetic acid, buffers such as acetates, citrates or phosphates: surfactants such as polysorbate 80; and agents for the adjustment of tonicity such as sodium chloride or dextrose. pH can be adjusted with acids or bases, such as hydrochloric acid or sodium hydroxide. In some embodiments, the pharmaceutical composition comprises a reductant. The presence of a reductant can help minim/ze potential radiolysis. In some embodiments, the reductant is ascorbic acid, gentisic acid, sodium thiosulfate, citric acid, tartaric acid, or a combination thereof.

In some embodiments, conjugates, or pharmaceutically acceptable salts or solvates thereof, and pharmaceutical compositions described herein are administered via intravenous administration. In some embodiments, the pharmaceutical composition is formulated for intravenous administration.

Pharmaceutical compositions comprising the conjugates or pharmaceutically acceptable salts or solvates thereof described herein can be prepared according to standard techniques and further comprise a pharmaceutically acceptable carrier. In some embodiments, normal saline can be employed as the pharmaceutically acceptable carrier. Other suitable carriers include, e.g., water, buffered water, 0.9% isotonic saline, 0.4% saline, 0.3% glycine, and the like, including glycoproteins for enhanced stability, such as albumin. lipoprotein, globulin, etc. These compositions can be sterilized by conventional sterilization techniques. The resulting aqueous solutions may be packaged for use or filtered under aseptic conditions and lyophilized. In some embodiments, the lyophilized preparation is combined with a sterile aqueous solution prior to administration. The compositions can contain pharmaceutically acceptable auxiliary substances as appropriate to approximate physiological conditions, such as pH adjusting and buffering agents, tonicity adjusting agents and the like, for example, sodium acetate, sodium lactate, sodium chloride, potassium chloride, calcium chloride, sodium lactate, sorbitan monolaurate, triethanolamine oleate, etc. Pharmaceutical compositions can be selected according to their physical characteristic, including, but not limited to fluid volumes, viscosities and other parameters in accordance with the particular mode of administration selected. The amount of conjugates administered can depend upon the particular targeting moiety used, the disease state being treated, the therapeutic agent being delivered, and the judgment of the clinician.

The concentration of the conjugates or pharmaceutically acceptable salts or solvates thereof described herein in the pharmaceutical formulations can vary. In some embodiments, the conjugate is present in the pharmaceutical composition from about 0.05% to about 1% by weight, about 1% to about 2% by weight, about 2% to about 5% by weight, about 5% to about 10% by weight, about 10% to about 30% by weight, about 30% to about 50% by weight, about 50% to about 75% by weight, or about 75% to about 99% by weight.

Pharmaceutical compositions are administered in a manner appropriate to the disease to be treated. An appropriate dose and a suitable duration and frequency of administration will be determined by such factors as the condition of the subject, the type and severity of the subject's disease, the particular form of the active ingredient, and the method of administration. In some embodiments, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the subject.

The amount of conjugates or pharmaceutically acceptable salts or solvates thereof and/or pharmaceutical compositions administered can be sufficient to deliver a therapeutically effective dose of the particular subject. In some embodiments, conjugate dosages can be between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of the conjugate or a pharmaceutically acceptable salt or solvate thereof described herein for methods of treating a disease as described herein is about 0.001 mg/kg to about 1 mg/kg body weight of the subject per dose. In some embodiments, the dose of conjugate or a pharmaceutically acceptable salt or solvate thereof described herein for the described methods is about 0.001 mg to about 1000 mg per dose for the subject being treated. In some embodiments, a conjugate or a pharmaceutically acceptable salt or solvate thereof described herein is administered to a subject at a dosage of from about 0.01 mg to about 500 mg, from about 0.01 mg to about 100 mg, or from about 0.01 mg to about 50 mg. In some embodiments, a conjugate or a pharmaceutically acceptable salt or solvate thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole. In some embodiments, a conjugate or a pharmaceutically acceptable salt or solvate thereof described herein is administered to a subject at a dosage of about 0.0001 Gbq to about 1000 Gbq, 0.01 Gbq to about 1000 Gbq, about 0.5 Gbq to about 100 Gbq, or about 1 Gbq to about 50 Gbq. In some embodiments, a conjugate or a pharmaceutically acceptable salt or solvate thereof described herein is administered to a subject at a dosage of about 5kBq/kg to about 50,000kBq/kg body weight per dose. In some embodiments, a conjugate or a pharmaceutically acceptable salt or solvate thereof described herein is administered to a subject at a dosage of about 1kBq/kg to about 0.2GBq/kg body weight per dose. In some embodiments, a conjugate or a pharmaceutically acceptable salt or solvate thereof described herein is administered to a subject at a dosage of about 20k Bq/kg to about 5.000kBq/kg body weight per dose. In some embodiments, a conjugate or a pharmaceutically acceptable salt or solvate thereof described herein is administered to a subject at a dosage of about 50k Bq/kg to about 500 kBq/kg body weight per dose. In some embodiments, the dose is administered once a day, 1 to 3 times a week, 1 to 4 times a month, or 1 to 12 times a year.

The pharmaceutical formulations can be packaged in unit dosage form for ease of administration and uniformity of dosage. A unit dosage form can refer to physically discrete units suited as unitary dosages for the subject to be treated; each unit containing a predetermined quantity of active compound calculated to produce the desired therapeutic effect in association with the pharmaceutical carrier or excipient.

### V. Method of Use, Diagnosis, and Treatment

In one aspect, the disclosure provides methods of treating a disease or condition in a subject in need thereof. In some embodiments, the methods comprise administering a conjugate or a pharmaceutically acceptable salt or solvate thereof described herein, or a pharmaceutical composition comprising the same to the subject in need thereof. In some embodiments, provided herein is a method of providing a therapeutic and/or prophylactic benefit to a subject in need thereof comprising administering a compound or pharmaceutical composition described herein.

In some embodiments, the methods comprise administering to a subject a therapeutically effective amount of a conjugate or a pharmaceutically acceptable salt or solvate thereof. In some embodiments, the conjugate or pharmaceutically acceptable salt or solvate thereof is administered in a pharmaceutical composition. In some embodiments, the subject has cancer. In some embodiments, the cancer is a solid tumor or hematological cancer.

In one aspect, provided herein are methods for killing a cell comprising contacting the cell with a conjugate (or a pharmaceutically acceptable salt or solvate thereof) or a pharmaceutical composition comprising the same, wherein the cell expresses a mutated KRAS protein having G12C mutation. In one aspect, provided herein are methods for delivering a radionuclide to a cell comprising administering a conjugate (or a pharmaceutically acceptable salt or solvate thereof) or a pharmaceutical composition comprising the same, wherein the cell expresses a mutated KRAS protein having G12C mutation. After contacting a cell, the described conjugate can permeate into the cell. In some embodiments, the conjugate or pharmaceutically acceptable salt or solvate thereof binds to mutated intracellular protein KRAS in a cell.

In some embodiments, provided herein are methods of making a covalently modified KRAS protein in vivo comprising administering a radiolabeled compound (or a pharmaceutically acceptable salt or solvate thereof) or a pharmaceutical composition comprising the same as described herein, to a subject having a KRAS G12C mutation.

In some embodiments, provided herein are methods for killing a cell comprising contacting the cell with a conjugate or a pharmaceutically acceptable salt or solvate thereof, wherein the cell expresses a mutated KRAS protein having G12C mutation. In one aspect, provided herein are methods for killing a cell harboring a mutated KRAS protein with a G12C mutation, the method comprising contacting the cell with a conjugate (or a pharmaceutically acceptable salt or solvate thereof) or a pharmaceutical composition comprising the same, thereby delivering a dose of radiation to the cell. In some embodiments, the conjugate or pharmaceutically acceptable salt or solvate thereof binds to a structure inside the cell. In some embodiments, the conjugate or pharmaceutically acceptable salt or solvate thereof releases a number of alpha particles by natural radioactive decay. In some embodiments, the conjugate or pharmaceutically acceptable salt or solvate thereof releases a number of beta particles, gamma rays, and/or Auger electrons by natural radioactive decay. The conjugate described herein can kill a cell by radiation. In some embodiments, the conjugate kills the cell directly by radiation. In some embodiments, the radiation creates, in the cell, oxidized bases, abasic sites, single-stranded breaks, double-stranded breaks. DNA crosslink, chromosomal rearrangement, or a combination thereof. In some embodiments, the conjugate kills the cell by inducing double-stranded DNA breaks. In some embodiments, the released alpha particles are sufficient to kill the cell. In some embodiments, the released alpha particles are sufficient to stop cell growth. In some embodiments, the conjugate kills the cell indirectly via the production of reactive oxygen species (ROS) such as free hydroxy 1 radicals. In some embodiments, the conjugate kills the cell indirectly by releasing tumor antigens from one or more different cells, which can have vaccine effect. In some embodiments, the conjugate kills the cell by abscopal effect. In some embodiments, the cell is a cancer cell. In some embodiments, the method comprises killing a cell with an alpha-particle emitting radionuclide.

In one aspect, provided herein are methods for diagnosing cancer patients harboring a KRAS G12C mutation comprising administering to a patient a conjugate described herein (or a pharmaceutically acceptable salt or solvate thereof) or a pharmaceutical composition comprising the same. In one aspect, provided herein are methods for imaging a cancer harboring a G12C KRAS mutation comprising administering to a patient a conjugate described herein (or a pharmaceutically acceptable salt or solvate thereof) or a pharmaceutical composition comprising the same. In some embodiments, the method further comprises selecting or confirming that a tumor in the patient has a G12C mutation. In some embodiments, the method further comprises measuring the concentration of the conjugate accumulated in the patient. In some embodiments, the method further comprises measuring the amount of radiation emitted from the radionuclide. In some embodiments, the method further comprises analyzing the elimination or clearance profile of the conjugate in the patient. In some embodiments, the method further comprises measuring an elimination half-life of the conjugate in the patient. In some embodiments, the method further comprises analyzing the clearance profile of the conjugate in the patient. In some embodiments, the method of imaging or diagnosing cancer comprises administering a conjugate that comprises a radionuclide of Table 4B, such as ⁶⁸Ga. For example, conjugates of the present disclosure can be administered for patient selection purposes, such as to confirm the tumor has the appropriate expression of the G12C target. As another example, conjugates of the present disclosure can be administered to a patient so that the patient's care team can make sure the conjugate is cleared from the body in a suitable timeframe so that undesired irradiation of other tissues is minim/zed.

In some embodiments, a method described herein comprises administering to a patient two conjugates of the present disclosure. In some embodiments, the two conjugates can have the same targeting ligand and/or linker. In some embodiments, a method described herein comprises administering (i) a conjugate of the present disclosure that comprises a radionuclide of Table 6B, and followed by (ii) a conjugate of the present disclosure that comprises a radionuclide of Table 6A. In some embodiments, a method described herein comprises administering (i) a conjugate of the present disclosure that comprises a radionuclide of Table 6D, and followed by (ii) a conjugate of the present disclosure that comprises a radionuclide of Table 6C.

In one aspect, the disclosed conjugate or a pharmaceutically acceptable salt or solvate thereof is configured to treat cancer by ablating tumor cells. In some embodiments, the conjugate or a pharmaceutically acceptable salt or solvate thereof does not modulate the biology of the tumor cell and/or the surrounding stroma. In some embodiments, the conjugate or a pharmaceutically acceptable salt or solvate thereof does not modulate immune cells. In some embodiments, the ablating of tumor cells can lead to a downstream immumological cascade.

Further provided herein are methods of treating a cancer associated with a KRAS G12C mutation. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a subject or population of subjects to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a subject or population of subjects to be treated with a pharmaceutical composition of the present disclosure have a hematological cancer. In some embodiments, the subject has a hematological cancer such as Diffuse large B cell lymphoma (“DLBCL”), Hodgkin's lymphoma (“HL”). Non-Hodgkin's lymphoma (“NHL”). Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer and melanoma.

In some embodiments, provided herein are methods and compositions for treating a disease or condition associated with KRAS G12C mutation. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, hepatocellular cancer, colorectal cancer, gastric adenocarcinoma, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunics conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer.

In some embodiments, a cancer to be treated by the methods of the present disclosure include, for example. Cardiac cancer: sarcoma (angiosarcoma, fibrosarcoma, rhabdomyosarcoma, liposarcoma), myxoma, rhabdomyoma, fibroma, lipoma and teratoma; Lung cancer: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal cancer: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors. Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract cancer: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma). Liver cancer: hepatoma (hepatocellular carcinoma), cholangiocarcinoma, hepatoblastoma, angiosarcoma, hepatocellular adenoma, hemangioma; Biliary tract cancer: gall bladder carcinoma, ampullary carcinoma, cholangiocarcinoma; Bone cancer: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system cancer: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological cancer: uterus (endometrial carcinoma (serous cystadenocarcinoma, mucinous cystadenocarcinoma, unclassified carcinoma), granulosa-thecal cell tumors. Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic cancer: blood (myeloid leukemia (acute and chronic), acute lymphoblastic leukemia, chronic lymphocytic leukemia, myeloproliferative diseases, multiple myeloma, myelodysplastic syndrome). Hodgkin's disease, non-Hodgkin's lymphoma (malignant lymphoma): Skin cancer: malignant melanoma, basal cell carcinoma, squamous cell carcinoma, Kaposi's sarcoma, moles dysplastic nevi, lipoma, angioma, dermatofibroma, keloids, psoriasis; and Adrenal glands cancer: neuroblastoma. In some embodiments, the cancer is non-small cell lung cancer.

In some embodiments, a conjugate described herein can be administered alone or in combination with one or more additional therapeutic agents. For example, the combination therapy can include a composition comprising a conjugate described herein co-formulated with, and/or co-administered with, one or more additional therapeutic agents. e.g., one or more anti-cancer agents. e.g., cytotoxic or cytostatic agents, immune checkpoint inhibitors, hormone treatment, vaccines, and/or immunotherapies. In some embodiments, the conjugate is administered in combination with other therapeutic treatment modalities, including surgery, cryosurgery, and/or chemotherapy. Such combination therapies may advantageously utilize lower dosages of the administered therapeutic agents, thus avoiding possible toxicities or complications associated with the various monotherapies.

When administered in combination, two (or more) different treatments can be delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective. e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the herein-described conjugate is used in combination with a chemotherapeutic agent, e.g., a DNA damaging chemotherapeutic agent. Non-limiting examples of DNA damaging chemotherapeutic agents include topoisomerase I inhibitors, topoisomerase II inhibitors; alkylating agents; DNA intercalators: DNA intercalators and free radical generators such as bleomycin; and nucleoside mimetics. In some embodiments, the herein-described conjugate is used in combination with a radiation sensitizer, which makes tumor cells more sensitive to radiation therapy. In some embodiments, the herein-described conjugate is used in combination with a DNA damage repair inhibitor (or DNA damage response (DDR) inhibitor).

In some embodiments, provided herein are methods of producing a compound having a structure of Formula (VIa), Formula (VIb), Formula (VIc), or Formula (VId) in vivo, comprising administering a radiolabeled compound, or pharmaceutically salt or solvate thereof, as described herein, to a subject.

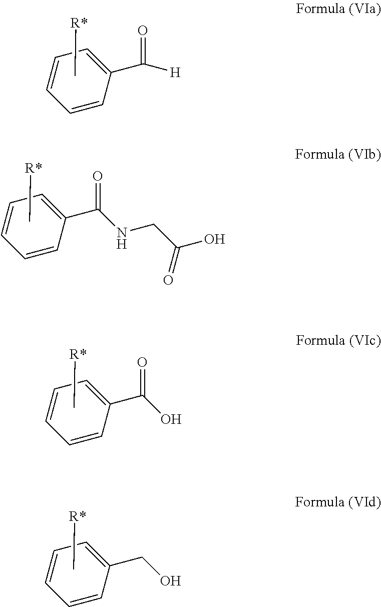

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, R* is selected from a radioisotope in Table 6C or Table 6D. In some embodiments, the method comprises administering a radiolabeled compound of Formula (III), Formula (IIIa), Formula (IIIa-1), Formula (IIIa-2), Formula (IIIb), Formula (IIIc), Formula (IV), Formula (IVa). Formula (IVb), Formula (IVc), Formula (IVd), or Formula (IVe).

In some embodiments, provided herein are methods of excreting a compound having a structure of Formula (VIa), Formula (VIb), Formula (VIc), or Formula (VId) from a subject's body, comprising administering a radiolabeled compound, or pharmaceutically salt or solvate thereof, as described herein, to the subject,

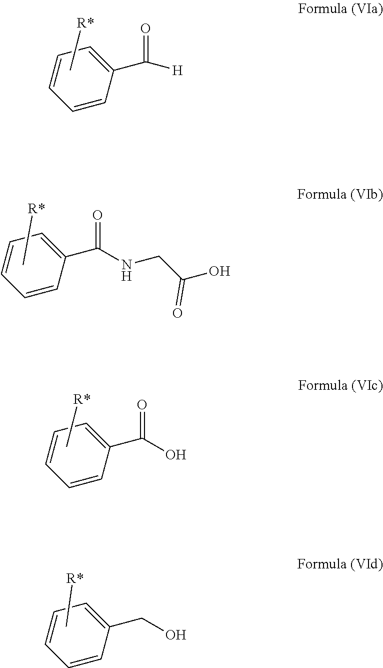

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, R* is selected from a radioisotope in Table 6C or Table 6D. In some embodiments, the method comprises administering to the subject a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe).

In some embodiments, the compound of formula (VIa) is

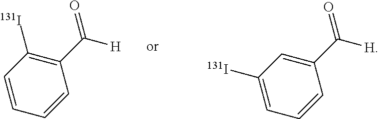

In some embodiments, the compound of formula (VIb) is

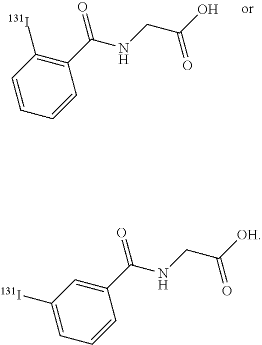 [SMILES: O=C(O)CNC(=O)c1ccccc1[131I] ; O=C(O)CNC(=O)c1cccc([131I])c1]

In some embodiments, the compound of formula (VIc) is

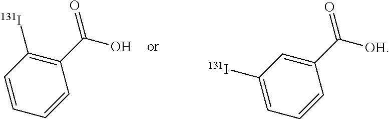 [SMILES: O=C(O)c1ccccc1[131I] ; O=C(O)c1cccc([131I])c1]

In some embodiments, the compound of formula (VId) is

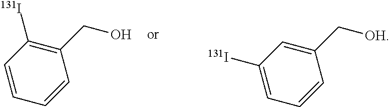 [SMILES: OCc1ccccc1[131I] ; OCc1cccc([131I])c1]

In some embodiments, the subject is 4 to 100 years old. In some embodiments, the subject is 5 to 10, 5 to 15.5 to 18, 5 to 25.5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45.25 to 55, 25 to 65, 25 to 75.35 to 45, 35 to 55.35 to 65.35 to 75, 45 to 55.45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

In addition to the methods of treatment described above, the compounds and compositions described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes such as the isotopes in Table 6B or 6D. Accordingly, the conjugate can be administered as a companion diagnostic.

In some embodiments, provided herein are methods of producing a compound having a structure of Formula (VIa), Formula (VIb), Formula (VIc), or Formula (VId) in vivo, comprising administering a radiolabeled compound, or pharmaceutically salt or solvate thereof, as described herein, to a subject,

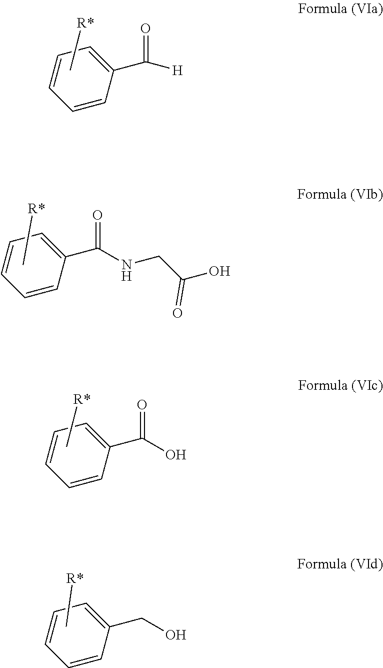

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, R* is selected from a radioisotope in Table 6C or Table 6D. In some embodiments, the method comprises administering to the subject a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe).

In some embodiments, provided herein are methods of excreting a compound having a structure of Formula (VIa), Formula (VIb), Formula (VIc), or Formula (VId) from a subject's body, comprising administering a radiolabeled compound, or pharmaceutically salt or solvate thereof, as described herein, to the subject.

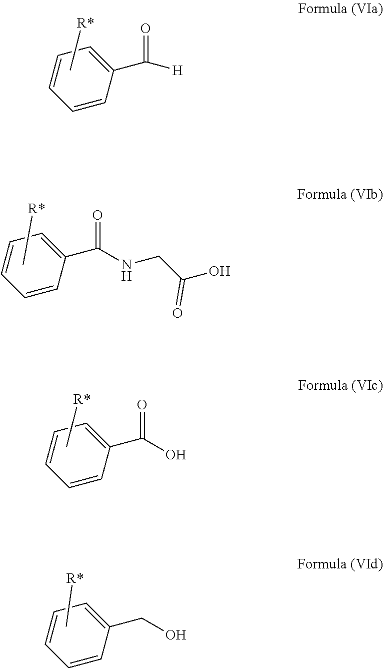

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At). In some embodiments, R* is selected from a radioisotope in Table 6C or Table 6D. In some embodiments, the method comprises administering to the subject a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe).

In some embodiments, the compound of formula (VIa) is

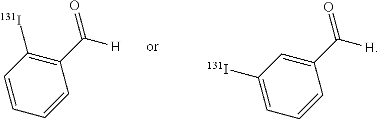

In some embodiments, the compound of formula (VIb) is

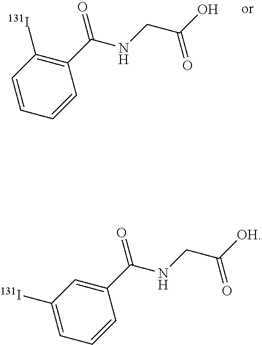 [SMILES: O=C(O)CNC(=O)c1ccccc1[131I] ; O=C(O)CNC(=O)c1cccc([131I])c1]

In some embodiments, the compound of formula (IVc) is

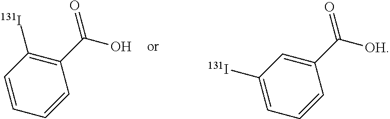 [SMILES: O=C(O)c1ccccc1[131I] ; O=C(O)c1cccc([131I])c1]

In some embodiments, the compound of formula (VId) is

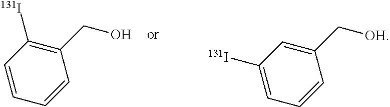 [SMILES: OCc1ccccc1[131I] ; OCc1cccc([131I])c1]

### VI. Method of Manufacturing Labelled Compounds

In some embodiments, radiolabeled compounds described herein can be synthesized from a boronic acid, boronate, or stannane precursor. Boronic acid, boronate, and stannane precursors can be formed according to the following general reaction Scheme 1:

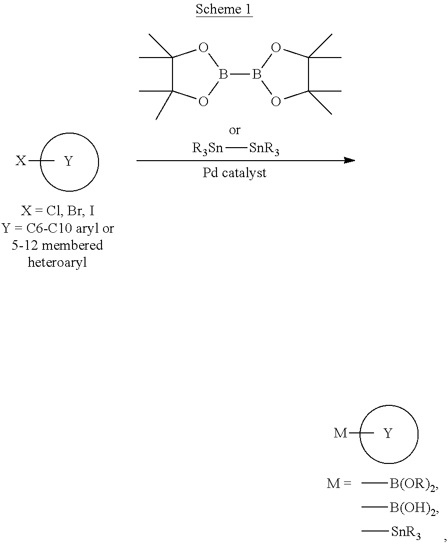

where R is a variable chemical moiety, for example alkyl or hydrogen. A halogen on ring Y, for example chloro, bromo, or iodo, can undergo a palladium mediated coupling reaction with a boronic acid, boronate, or stannane compound to form an intermediate compound wherein group M has replaced halogen X.

A radioisotope, for example R* such as fluorine-18 (¹⁸F), iodine-131 (¹³¹I), iodine-123 (¹²³I), iodine-124 (¹²⁴I), iodine-125 (¹²⁵I), or astatine-211 (²¹¹At), or any other radioisotope of Table 6C and Table 6D can be formed, for example, from the intermediate compound according to the following general reactions:

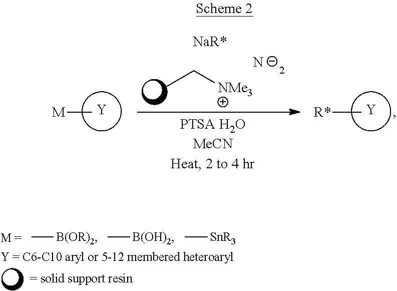

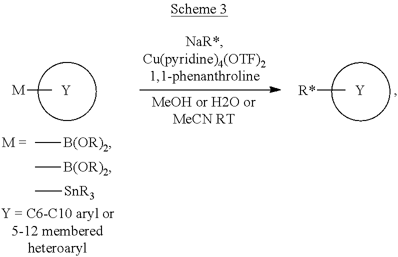

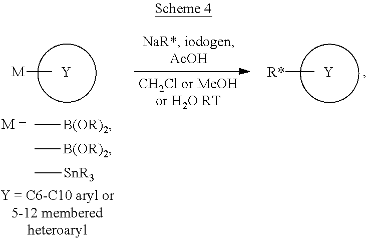

where R is a variable chemical moiety, for example alkyl or hydrogen.

In some embodiments, radiolabeled compounds described herein can be synthesized from a chloro, bromo, or iodo precursor according to the following general reaction:

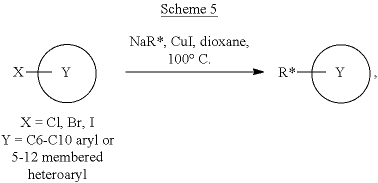

where R* is a radioisotope, for example, fluorine-18 (¹⁸F), iodine-131 (¹³¹I), iodine-123 (¹²³), iodine-124 (¹²⁴I), iodine-125 (¹²⁵I), astatine-211 (²¹¹At), or a radioisotope of Table 6C and Table 6D.

The radiolabeling reactions depicted above are used as example procedures in the synthesis of radiolabeled compounds described herein. Additional reactions, including nucleophilic substitution, electrophilic substitution, isotopic exchanges, bromine-radioiodine exchange, radioiododestannylation, radioiododeboronation, and transition metal mediated halogen exchange are contemplated and procedures can be found in Berdal et al., “Investigation on the reactivity of nucleophilic radiohalogens with arylboronic acids in water: access to an efficient single-step method for the radioiodination and astatination of antibodies” Chemical Science 2021, 12, 1458, and Dubost et al., “Recent Advances in Synthetic Methods for Radioiodination” J. Org. Chem. 2020, 85, 13, 8300-8310, both of which are incorporated by reference herein in their entirety.

Accordingly, in one aspect, described herein is a method of synthesizing a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe) or salt or solvate or pharmaceutical composition thereof. In some embodiments, the method comprises replacing a halogen on a precursor compound with a radioisotope, e.g., a fluorine-18 (¹⁸F), iodine-131 (¹³¹I), iodine-123 (¹²³I), iodine-124 (¹²⁴I), iodine-125 (¹²⁵I), astatine-211 (²¹¹At), or a radioisotope of Table 6C and Table 6D. In some embodiments, the method comprises a transition metal (e.g., palladium) mediated coupling reaction with a boronic acid, boronate, or stannane compound to form an intermediate compound. In some embodiments, the method further comprises exchanging the boronic acid, boronate, or stannane group with the radioisotope, e.g., as illustrated above. In some embodiments, the method comprises nucleophilic substitution, electrophilic substitution, isotopic exchanges, bromine-radioiodine exchange, radioiododestannylation, radioiododeboronation, and/or transition metal mediated halogen exchange reactions. Exemplary procedures of the radioisotope labelling steps are illustrated in Schemes 1-7.

In some embodiments, provided herein are precursor compounds of compounds of Formula (III). Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe), wherein the radioisotope is replaced with a halogen or hydrogen. In some embodiments, provided herein are precursor compounds having a structure of Formula (III′), Formula (IIIa′), Formula (IIIa-1′), Formula (IIIa-2′), Formula (IIIb′), Formula (IIIc′), Formula (IV′), Formula (IVa′), Formula (IVb′), Formula (IVc′), Formula (IVd′), or Formula (IVe′), each of which have the corresponding structure of Formula (III), Formula (IIIa), Formula (IIIb), Formula (IIIc), Formula (IV), Formula (IVa), Formula (IVb), Formula (IVc), Formula (IVd), or Formula (IVe), respectively, except that the precursor compounds contain a halogen, amine (e.g., NH₂), B(OH)₂, Si(Me)₃, Sn(Bu)₃, hydrogen or the like in lieu of the radioisotope R.

In some embodiments, provided herein are methods of synthesizing a compound of Formula (III), comprising: (i) reacting a precursor compound having a structure of Formula (III′)

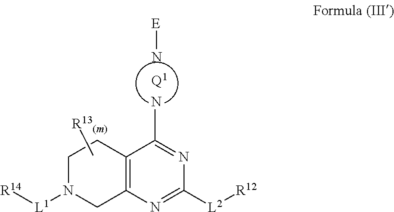

wherein the Q¹, E, R¹³, m, R¹⁴, L¹, L², and R¹² are defined in Formula (III), except that the radioisotope R* is replaced with a halogen (e.g., F, Cl, or I), amine (e.g., NH₂), B(OH)₂. Si(Me)₃, Sn(Bu)₃, hydrogen or the like in formula (III′),
and (ii) replacing the halogen with the R*.

In some embodiments, provided herein are methods of synthesizing a compound of Formula (IV), comprising: (i) reacting a precursor compound having a structure of Formula (IV′)

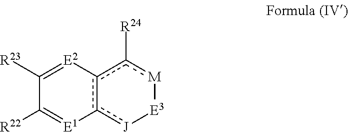

wherein the E¹, E², E³, M, R²², R²³, R²⁴, and J are defined in Formula (IV), except that the radioisotope R* is replaced with a halogen (e.g., F, Cl, or I), amine (e.g., NH₂), B(OH)₂, Si(Me)₃, Sn(Bu)₃, hydrogen or the like in formula (IV′),
and (ii) replacing the halogen with the R.

In some embodiments, provided herein is a precursor compound of Formula (III′)

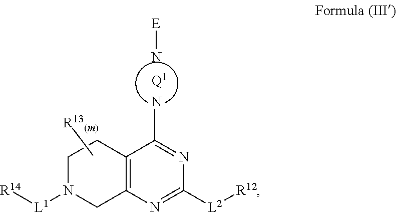

wherein the radioisotope R* is replaced with R*^{Pre}, and the remaining groups are defined in Formula (III), wherein R*^{Pre} is a precursor of a radioisotope. In some embodiments, R*^{Pre} can be a halogen (e.g., F, Cl, or I), amine (e.g., NH₂), B(OH)₂, Si(Me)₃, Sn(Bu)₃, hydrogen or the like.

In some embodiments, provided herein is a precursor compound of Formula (IIIa′)
wherein

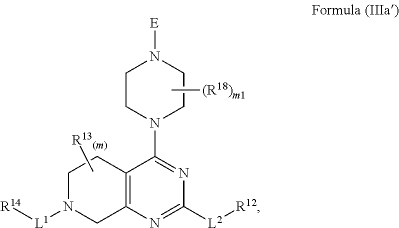

the radioisotope R* is replaced with R*^{Pre}, and the remaining groups are defined in Formula (IIIa), wherein R* is a precursor of a radioisotope.

In some embodiments, provided herein is a precursor compound of Formula (IIIa-1′)
wherein

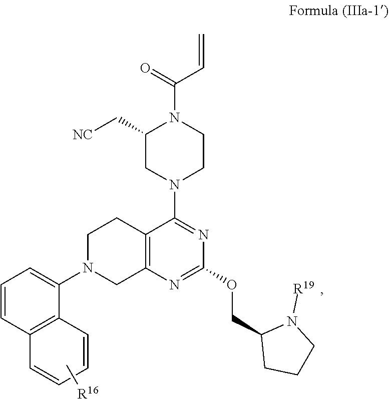

the radioisotope R* is replaced with R*^{Pre}, and the remaining groups are defined in Formula (IIIa-1).
wherein R*^{Pre} is a precursor of a radioisotope.

In some embodiments, provided herein is a precursor compound of Formula (IIIa-2′)

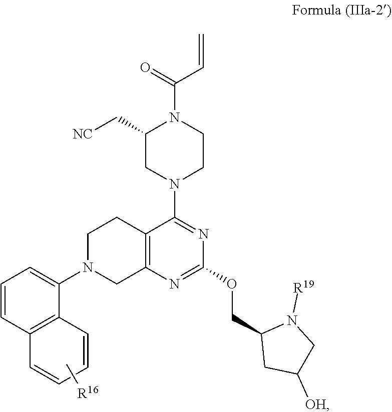

wherein
the radioisotope R* is replaced with R*^{Pre}, and the remaining groups are defined in Formula (IIIa-2),
wherein R*^{Pre} is a precursor of a radioisotope.

In some embodiments, provided herein is a precursor compound of Formula (IIIb′)

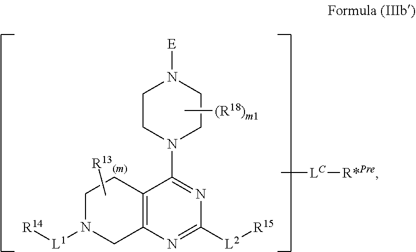

wherein R*^{Pre} is a precursor of a radioisotope and the remaining groups are defined in Formula (IIIb). In some embodiments, R*^{Pre} can be a halogen (e.g., F, Cl, or I), amine (e.g., NH₂), B(OH)₂, Si(Me)₃, Sn(Bu)₃, hydrogen or the like.

In some embodiments, provided herein is a precursor compound of Formula (IIIc′).

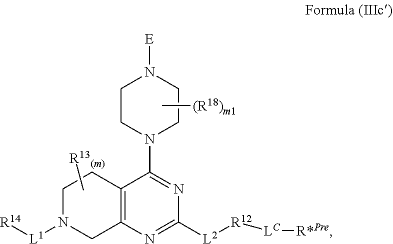

wherein R*^{Pre} is a precursor of a radioisotope and the remaining groups are defined in Formula (IIIc). In some embodiments, R*^{Pre} can be a halogen (e.g., F, Cl, or I), amine (e.g., NH₂), B(OH)₂, Si(Me)₃, Sn(Bu)₃, hydrogen or the like.

In some embodiments, provided herein is a precursor compound of Formula (IV′)

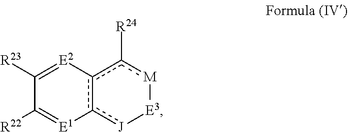

wherein
the radioisotope R* is replaced with R*^{Pre}, and the remaining groups are defined in Formula (IV), wherein R*^{Pre} is a precursor of a radioisotope. In some embodiments, R*^{Pre} can be a halogen (e.g., F, Cl, or I), amine (e.g., NH₂), B(OH)₂, Si(Me)₃, Sn(Bu)₃, hydrogen or the like.

In some embodiments, provided herein is a precursor compound of Formula (IVa′)

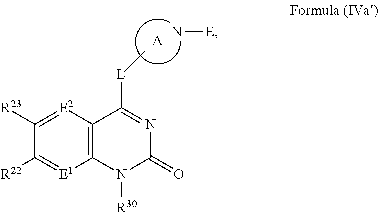

wherein
the radioisotope R* is replaced with R*^{Pre}, and the remaining groups are defined in Formula (IV), wherein R*^{Pre} is a precursor of a radioisotope.

In some embodiments, provided herein is a precursor compound of Formula (IVd′).

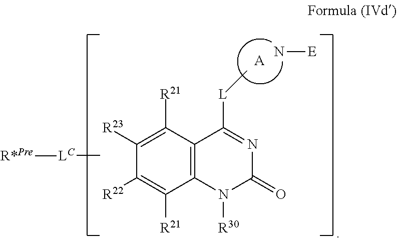

wherein R*^{Pre} is a precursor of a radioisotope and the remaining groups are defined in Formula (IVd). In some embodiments. R*^{Pre} can be a halogen (e.g., F, Cl, or I), amine (e.g., NH₂), B(OH)₂, Si(Me)₃, Sn(Bu)₃, hydrogen or the like.

In some embodiments, provided herein is a precursor compound of Formula (IVe′),

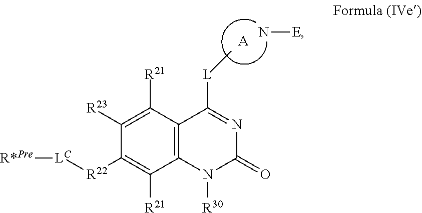

wherein R*^{Pre} is a precursor of a radioisotope and a remaining groups are defined in Formula (IVe). In some embodiments. R*^{Pre} can be a halogen (e.g., F, Cl, or I), amine (e.g., NH₂), B(OH)₂, Si(Me)₃, Sn(Bu)₃, hydrogen or the like.

It is to be understood that any groups or substituents (except R*) provided in this disclosure for any of the Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe) are also applicable to Formula (III′), Formula (IIIa′), Formula (IIIa-1′), Formula (IIIa-2′), Formula (III′), Formula (IIIc′), Formula (IV′), Formula (IVa′), Formula (IVb′), Formula (IVc′). Formula (IVd′), or Formula (IVe′), respectively, unless explicitly stated otherwise.

In some embodiments, R*^{Pre} is halogen (e.g., F, Cl, or I). In some embodiments, R*^{Pre} is I. In some embodiments, R*^{Pre} is Br. In some embodiments, R*^{Pre} is H. In some embodiments, R*^{Pre} is NH₂. In some embodiments, R*^{Pre} is B(OH)₂. In some embodiments, R*^{Pre} is Si(Me)₃. In some embodiments, R*^{Pre} is Si(C₁₋₄ alkyl)₃. In some embodiments, R*^{Pre} is Sn(Bu)₃. In some embodiments, R*^{Pre} is Sn (C₁₋₄ alkyl)₃. In some embodiments. R*^{Pre} is SnH₃. In some embodiments, R*^{Pre} is B(O-alkyl)₂ wherein the two alkyl groups can form a ring. In some embodiments, R*^{Pre} is boronate. In some embodiments, R*^{Pre} is B(pinacol).

Accordingly, in one aspect, described herein is a method of synthesizing a radiolabeled compound of Formula (III), Formula (IIIa), Formula (IIIa-1), Formula (IIIa-2), Formula (IIIb), Formula (IIIc), Formula (IV), Formula (IVa), Formula (IVb), Formula (IVc), Formula (IVd), or Formula (IVe) or salt or solvate thereof, comprising replacing a precursor group in a structure of Formula (III′), Formula (IIIa′), Formula (IIIa-1′), Formula (IIIa-2′), Formula (IIIb′), Formula (IIIc′), Formula (IV′), Formula (IVa′), Formula (IVb′), Formula (IVc′), Formula (IVd′), or Formula (IVe′), respectively, with a radioisotope R*.

In some embodiments, the reacting and replacing occur in a one step reaction, e.g., an isotopic exchange. In some embodiments, the isotopic exchange comprises bromine-radioiodine exchange. In some embodiments, the reacting and replacing comprise two or more steps. For example, compounds containing iodonium salts can be used as intermediates. In some embodiments, the reacting and replacing comprise direct electrophilic aromatic substitution. In some embodiments, the reacting and replacing comprise Silver(I) triflimide mediated electrophilic radioiodination. In some embodiments, the reacting and replacing comprise nickel mediated Halogen exchange. In some embodiments, the reacting and replacing comprise radioiododestannylation. Radioiododestannylation can be performed with any suitable organostannane compounds, e.g., with fluorine-rich organostannanes or ionic liquid supported organostannane. In some embodiments, the reacting and replacing comprise radioiododesilylation. In some embodiments, the reacting and replacing comprise electrophilic iododeboronation from boronic acids. In some embodiments, the reacting and replacing comprise KOAc-catalysed iododeboronation.

Although the present disclosure and its advantages have been described in detail, it should be understood that various changes, substitutions and alterations can be made herein without departing from the spirit and scope of the disclosure as defined in the appended claims. The present disclosure is further illustrated in the following Examples which are given for illustration purposes only and are not intended to limit the disclosure in any way.

### EXAMPLES

### A: Synthesis of the Compounds

### General Information

¹H NMR spectra were recorded on either a Bruker Avance III 400 (400 MHz), or Bruker Avance 300 (400 MHz) spectrometer. Chemical shifts are reported in ppm with solvent resonance as the internal standard (CDCl₃:7.27 ppm. DMSO-d₆: 2.50 ppm. CD₃OD: 3.31 ppm). Data are reported as follows: chemical shift, integration, multiplicity (s=singlet, d=doublet, t=triplet, q=quartet, p=pentet, m=multiples), and coupling constants (Hz).

Liquid chromatography was performed using forced flow (flash chromatography) on silica gel (SiO₂, 1000 mesh) or by column chromatography (silica gel, 1000 mesh). Thin layer chromatography (TLC) was performed on a 20-25 μm silica gel glass backed plates. Preparative TLC was performed on a 40-451 mm silica gel glass backed plates. Visualization was performed using ultraviolet light (254 nm), iodide, or KMnO₄ in water.

Reaction solvents Tetrahydrofuran (THF), dichloromethane (DCM), toluene, N, N-dimethylformamide (DMF), methanol (MeOH) and 1,4-dioxane were supplied by WuXi EHS department which were purified with Pure-Sole MD-6 solvent purification system (Innovation technology Limited), by passing the solvents through 4A molecular sieve column. All reaction reagents were purchased from Alfa Aesar, Aldrich or domestic vendors which were used without further purification.

### A1. Synthesis of Int 1

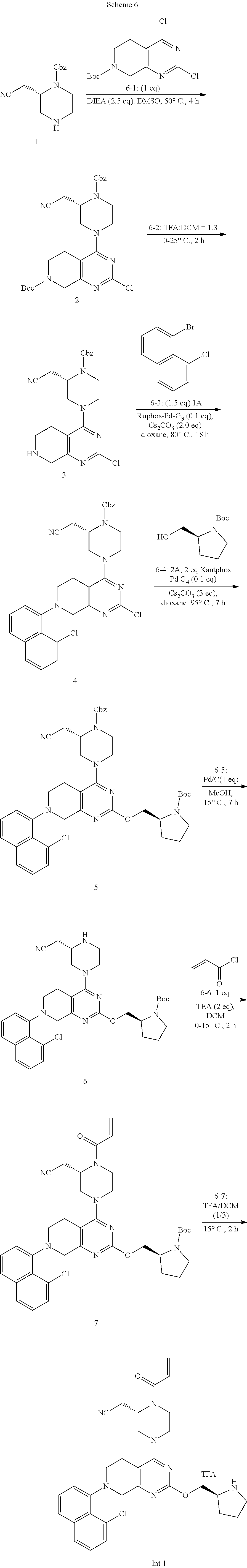

### 6.1 Preparation of Compound 2

To a solution of benzyl (2S)-2-(cyanomethyl)piperazine-1-carboxylate (14.5 g, 55.9 mmol, 1 eq) and tert-butyl 2,4-dichloro-6,8-dihydro-5H-pyrido[3,4-d]pyrimidine-7-carboxylate (17.0 g, 55.9 mmol, 1.0 eq) in DMSO (150 mL) was added DIEA (18.1 g, 140 mmol, 24.4 mL, 2.5 eq) and the mixture stirred at 50° C. for 12 h. The residue was diluted with water (300 mL), pH adjusted with the addition of a saturated NaHCO₃solution to pH=8-9 and extracted with EtOAc (150 mL×3). The combined organic layers were washed with brine, dried over Na₂SO₄, filtered, and concentrated under reduced pressure. The residue was purified by column chromatography (SiO₂, petroleum ethcr:EtOAc=20:1 to 3:1) to give tert-butyl 4-[(3S)-4-benzyloxycarbonyl-3-(cyanomethyl)piperazin-1-yl]-2-chloro-6,8-dihydro-5H-pyrido[3,4-d]pyrimidine-7-carboxylate (25.5 g, 48.4 mmol, 87% yield) as a brown oil; ¹H NMR (400 MHz, CHLOROFORM-d) δ ppm 1.49 (9H, s), 2.60-2.88 (4H, m), 3.11 (1H, td, J=12.20, 3.12 Hz), 3.23-3.48(3H, m), 3.74-3.92 (2H, m), 4.02-4.13 (2H, m), 4.39-4.49(1H, m), 4.60-4.74(2H, m), 5.20(2H, s), 7.39(5H, s).

### 6.2 Preparation of Compound 3

A mixture of tert-butyl 4-[(3S)-4-benzyloxycarbonyl-3-(cyanomethyl)piperazin-1-yl]-2-chloro-6,8-dihydro-5H-pyrido[3,4-d]pyrimidine-7-carboxylate (40.0 g, 75.9 mmol, 1 eq) and HCl (4 M, 113 mL, 6.0 eq) was degassed and purged with N₂ 3 times. The reaction was stirred at 0° C. for 1 h under a N₂ atmosphere. The solvent was concentrated under reduced pressure. The residue was diluted with water (200 mL). pH adjusted with saturated NaHCO₃solution to pH=8-9 and extracted with EtOAc (80 mL×3). The combined organic layers were washed with brine, dried over Na₂SO₄, filtered, and concentrated under reduced pressure to give crude benzyl (2S)-4-(2-chloro-5,6,7,8-tetrahydropyrido[3,4-d]pyrimidin-4-yl)-2-(cyanomethyl)piperazine-1-carboxylate (30.0 g) as a white solid; ¹H NMR (400 MHz, CHLOROFORM-d S ppm 2.60-2.74 (3H, m), 2.77-2.89(1H, m), 2.97-3.21 (3H, m), 3.23-3.40 (2H, m), 3.91 (1H, br d, J=12.8 Hz), 3.98-4.16(5H, m), 4.57-4.70(1H, m), 5.16-5.22 (2H, m), 7.32-7.45 (5H, m).

### 6.3 Preparation of Compound 4

A mixture of benzyl (2S)-4-(2-chloro-5,6,7,8-tetrahydropyrido[3,4-d]pyrimidin-4-yl)-2-(cyanomethyl)piperazine-1-carboxylate (15.0 g, 35.1 mmol, 1 eq), 1-bromo-8-chloro-naphthalene (21.2 g, 87.8 mmol, 2.5 eq), RuPhos Pd G3 (2.94 g, 3.51 mmol, 0.1 eq) and Cs₂CO₃ (34.4 g, 105 mmol, 3 eq) in dioxane (150 mL) was degassed and purged with N₂ 3 times, and the mixture stirred at 100° C. for 16 h under a N₂ atmosphere. The reaction mixture was filtered, concentrated under reduced pressure and purified by column chromatography (SiO₂, petroleum ether:EtOAc=10:1 to 5:1) to give benzyl (2S)-4-[2-chloro-7-(8-chloro-1-naphthyl)-6,8-dihydro-5H-pyrido[3,4-d]pyrimidin-4-yl]-2-(cyanomethyl)piperazine-1-carboxylate (15.0 g, 25.5 mmol, 73% yield) as a white solid; 1H NMR (400 MHz, CHLOROFORM-d) δ ppm 2.53-3.00(3H, m), 3.02-3.37(4H, m), 3.42-3.64(2H, m), 3.83-4.06 (2H, m), 4.12-4.24 (2H, m), 4.47 (1H, dd, J=18.2, 11.4 Hz), 4.68 (1H, hr s) 5.17-5.26 (2H, m), 7.17-7.25 (1H, m), 7.32-7.49 (7H, m), 7.51-7.56(1H, m), 7.63 (1H, dd, J=7.4, 5.3 Hz), 7.76 (1H, d, J=8.1 Hz).

### 6.4 Preparation of Compound 5

A mixture of benzyl (2S)-4-[2-chloro-7-(8-chloro-1-naphthyl)-6,8-dihydro-5H-pyrido[3,4-d]pyrimidin-4-yl]-2-(cyanomethyl)piperazine-1-carboxylate (3.0 g, 5.10 mmol, 1 eq), tert-butyl (2S)-2-(hydroxymethyl)pyrrolidine-1-carboxylate (1.23 g, 613 μmol, 1.2 eq), Cs₂CO₃(3.3 g, 1.02 mmol, 2 eq) and Xantphos Pd G4 (491 mg, 51.1 μmol, 0.1 eq) in dioxane (10 mL) was degassed and purged with N₂ 3 times. The reaction was stirred at 95° C. for 4 h under a N₂ atmosphere. The reaction mixture was filtered, concentrated under reduced pressure and purified by column chromatography (SiO₂, petroleum ether:EtOAc=20:1 to 5:1) to give benzyl (2S)-4-[2-[[(2S)-1-tert-butoxycarbonylpyrrolidin-2-yl]methoxy]-7-(8-chloro-1-naphthyl)-6,8-dihydro-5H-pyrido[3,4d]pyrimidin-4-yl-2-(cyanomethyl)piperazine-1-carboxylate (2.23 g, 3.1 mmol, 80% yield) as a white solid; 1H NMR (400 MHz, CHLOROFORM-d) δ ppm 1.40-1.46 (9H, m), 1.75-2.04 (5H, m), 2.44-2.60(1H, m), 2.72-2.86 (1H, m), 2.93-3.23 (4H, m), 3.29-3.46(3H, m), 3.57 (1H, br s), 3.75-4.12 (6H, m), 4.14-4.17 (2H, m), 4.59-4.78(1H, m), 5.21(2H, s), 7.19(1H, br d, J=7.2 Hz), 7.30-7.48(7H, m), 7.50-7.55(1H, m), 7.58-7.66 (1H, m), 7.75 (1H, br d, J=8.2 Hz).

### 6.5 Preparation of Compound 6

A mixture of benzyl (2S)-4-[2-[[((2S)-1-tert-butoxycarbonylpyrrolidin-2-yl]methoxy]-7-(8-chloro-1-naphthyl)-6,8-dihydro-5H-pyrido[3,4-d]pyrimidin-4-yl]-2-(cyanomethyl)piperazine-1-carboxylate (14.0 g, 18.6 mmol, 1.0 eq), Pd/C (19.7 g, 18.6 mmol, 10% purity, 1 eq), ZnBr₂ (419 mg, 1.86 mmol, 93 μL, 0.1 eq) and NH₂·H₂O (261 mg, 1.86 mmol, 287 μL, 25% purity, 0.1 eq) in MeOH (20 mL) was degassed and purged with N₂ 3 times. The reaction was stirred at 20° C. for 12 h under a N₂ atmosphere. The reaction mixture was filtered and concentrated under reduced pressure to give tert-butyl (2S)-2-[[7-(8-chloro-1-naphthyl)-4-[(3 S)-3-(cyanomethyl)piperazin-1-yl]-6,8-dihydro-5H-pyrido[3,4d]pyrimidin-2-yl]oxymethyl]pyrrolidine-1-carboxylate (9.0 g, 14.6 mmol, 78% yield) as a white solid: MS (ESI) m/z: 618.5 (M+H)⁺, Rₜ=0.80 min.

### 6.6 Preparation of Compound 7

To a solution of tert-butyl (2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidine-1-carboxylate (2.0 g, 3.24 mmol, 1 eq) in DCM (20 mL) was added prop-2-enoyl chloride (351 mg, 3.88 mmol, 317 μL, 1.2 eq) and TEA (982 mg, 9.71 mmol, 1.35 mL, 3 eq). The mixture was stirred at 0° C. for 1 h under a N₂ atmosphere. The reaction mixture was filtered, concentrated under reduced pressure and purified by column chromatography (SiO₂, petroleum ether:EtOAc=10:1 to 0:1) to give tert-butyl (2S)-2-([7-(8-chloro-1-naphthyl)-4-[(3S)-3-(cyanomethyl)-4-prop-2-enoyl-piperazin-1-yl]-6,8-dihydro-5H-pyrido[[3,4-d]pyrimidin-2-yl]oxymethyl]pyrrolidine-1-carboxylate (1.8 g, 2.68 mmol, 83% yield) as a white solid: ¹H NMR (400 MHz, CDCl₃) S (ppm) 1.43-1.48 (9H, m), 1.53-1.72 (2H, m), 1.81-2.03 (4H, m), 2.59(1H, d, J=1.6 Hz), 2.80-3.00 (2H, m), 3.11-3.27 (3H, m), 3.27-3.43 (3H, m), 3.50-3.66 (2H, m), 3.94 (2H, s), 4.16 (3H, d, J=8.0 Hz), 4.47 (2H, s), 5.82 (1H, d, J=10.4 Hz), 6.36-6.44 (1H, m), 6.54-6.61 (1H, m), 7.18-7.26 (1H, m), 7.33 (1H, t, J=7.6 Hz), 7.43 (1H, s), 7.52 (1H, d, J=7.6 Hz), 7.61 (1H, d, J=7.6 Hz), 7.75 (1H, d, J=8.0 Hz). MS (ESI) m/z: 672.4 (M+H)⁺, Rₜ=0.69 min.

### 6.7 Preparation of Compound Intermediate 1

To a mixture of tert-butyl (2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrolidine-1-carboxylate 3 (2.4 g, 3.57 mmol, 1.0 eq) in DCM (18 mL) was added TFA (9.24 g, 81.0 mmol, 6 mL, 23 eq) and the mixture stirred at 0° C. for 2 h. The mixture was diluted with DCM (30 mL), washed with a solution of Na₂CO₃ (50 mL), brine (30 mL), dried over Na₂SO₄, filtered and concentrated under reduced pressure. The residue was purified by prep-HPLC to give 2-[(2S)-4-[7-(8-chloro-1-naphthyl)-2-[[(2S)-pyrolidin-2-yl]methoxy]-0,8-dihydro-5H-pyrido[3,4-d]pyrimidin-4-yl]-1-prop-2-enoyl-piperazin-2-yl]acetonitrile TFA salt (1.25 g, 1.82 mmol, 51% yield) as a brown solid; ¹H NMR (400 MHz, CDCl₃) δ (ppm) 1.89-2.28 (5H, m), 2.68 (2H, d, J=14.4 Hz), 2.95 (1H, dd, J=15.6, 8.4 Hz), 3.11-3.43 (4H, m), 3.45-3.62 (4H, m), 3.88-4.12 (3H, m), 4.49 (3H, dd, J=18.4, 11.2 Hz), 4.75-4.91 (2H, m), 4.96-5.10 (1H, m), 5.86 (1H, d, J=10.0 Hz), 6.37-6.46 (1H, m), 6.51-6.63 (1H, m), 7.25 (1H, s), 7.36 (1H, t, J=7.6 Hz), 7.47 (1H, t, J=7.6 Hz), 7.55 (1H, d, J=7.6 Hz), 7.69 (1H, d, J=8.0 Hz), 7.78 (1H, d, J=8.0 Hz). MS (ESI) m/z: 572.4 (M+H)⁺, Rₜ=0.71 min.

### A2. Synthesis of Int 2

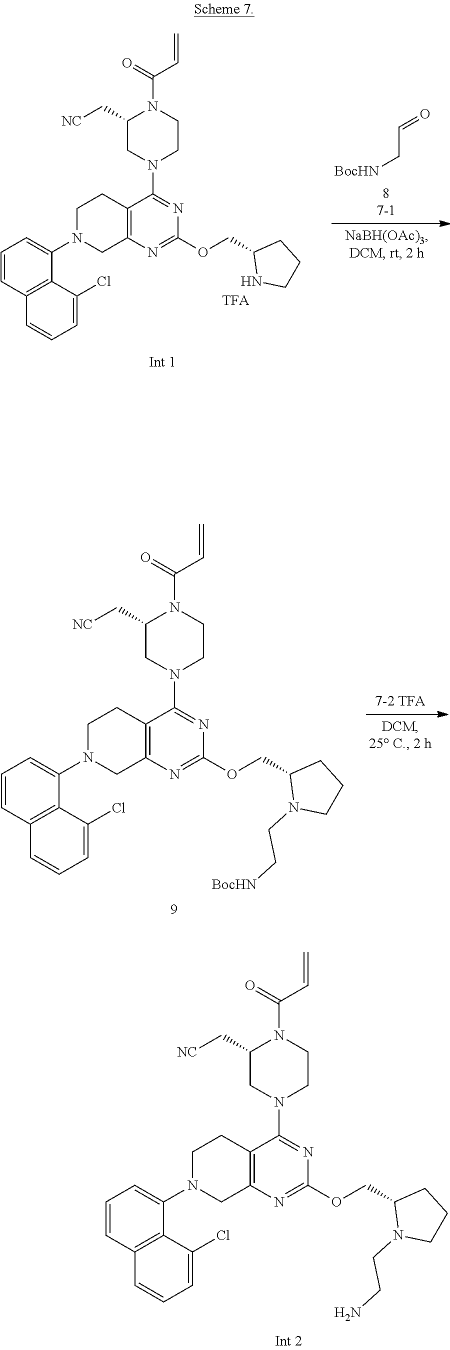 [SMILES: C=CC(=O)N1CCN(c2nc(OC[C@@H]3CCCN3)nc3c2CCN(c2cccc4cccc(Cl)c24)C3)C[C@@H]1CC#N ; CC(C)(C)OC(=O)NCC=O ; C=CC(=O)N1CCN(c2nc(OC[C@@H]3CCCN3CCNC(=O)OC(C)(C)C)nc3c2CCN(c2cccc4cccc(Cl)c24)C3)C[C@@H]1CC#N ; C=CC(=O)N1CCN(c2nc(OC[C@@H]3CCCN3CCN)nc3c2CCN(c2cccc4cccc(Cl)c24)C3)C[C@@H]1CC#N]

### 7.1 Preparation of Compound 9

2-[(2S)-4-[7-(8-chloro-1-naphthyl)-2-[[(2-S)-pyrrolidin-2-yl]methoxy]-6,8-dihydro-5H-pyrido[3,4-d]pyrimidin-4-yl]-1-prop-2-enoyl-piperazin-2-yl]acetonitrile TFA salt (250 mg, 364 umol, 1 eq) in DCM (5 mL) was cooled to 0° C., NaBH(OAc)₃ (116 mg, 547 umol, 1.5 eq), TEA (111 mg, 1.09 mmol, 152 uL, 3 eq) and tert-butyl N-(2-oxoethyl)carbamate (70 mg, 437 umol, 1.2 eq) added and the reaction stirred at 25° C. for 1 h. The reaction was diluted with DCM (20 mL), washed with brine (30×2 mL), dried over Na₂SO₄, filtered and concentrated under reduced pressure. The residue was purified by flash chromatography (DCM:MeOH=100:1 to 20:1) to give tert-butyl N-[2-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]ethyl]carbamate (150 mg, 210 umol, 58% yield) as a yellow solid; ¹H NMR (400 MHz. METHANOL-d₄) δ ppm 1.45 (9H, s), 1.73-1.94 (3H, m), 2.07-2.21 (1H, m), 2.51-2.81 (3H, m), 2.86-2.97(1H, m), 3.06-3.31 (8H, m), 3.44-3.66 (2H, m), 3.66-3.83 (1H, m), 4.09 (1H, s), 4.15 (2H, s), 4.29-4.46 (3H, m), 4.49-4.78 (2H, m), 4.50-4.87 (3H, m), 5.02-5.23 (1H, m), 5.75-6.03 (1H, m), 6.23-6.51 (1H, m), 6.71-6.99(1H, m), 7.30-7.43 (2H, m), 7.46-7.58 (2H, m), 7.67-7.72 (1H, m), 7.80-7.90 (1H, m). MS(ESI)ₙ/z: 715.6 (M+H)⁺, Rₜ=0.89 mm.

### 7.2 Preparation of Compound Int. 2

TFA (924 mg, 8.10 mmol, 600 uL, 39 eq) was added to a solution of tert-butyl N-[2-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]ethyl]carbamate (0.15 g, 210 umol, 1 eq) in DCM (3 mL) at 0° C. The reaction was stirred at 25° C. for 1 h and concentrated under reduced pressure to give crude 2-[(2S)-4-[2-[[(2S)-1-(2-aminoethyl)pyrolidin-2-yl]methoxyl]-7-(8-chloro-1-naphthyl)-6,8-dihydro-5H-pyrido[3,4-d]pyrimidin-4-yl]-1-prop-2-enoyl-piperazin-2-yl]acetonitrile as a yellow solid: MS (ES) m/z: 615.6 (M+H)⁺, Rₜ=0.82 mm.

### A3. Synthesis of Int. 3

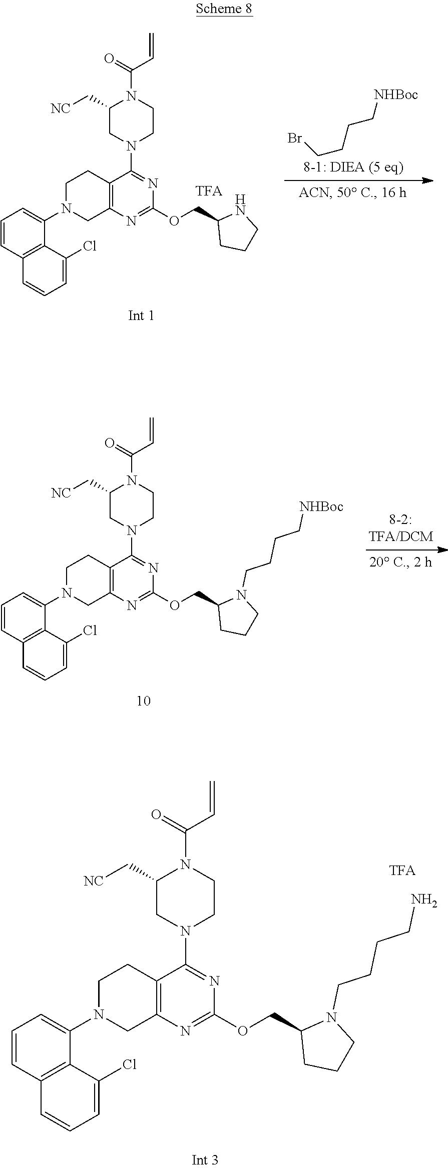 [SMILES: C=CC(=O)N1CCN(c2nc(OC[C@@H]3CCCN3)nc3c2CCN(c2cccc4cccc(Cl)c24)C3)C[C@@H]1CC#N ; CC(C)(C)OC(=O)NCCCCBr ; C=CC(=O)N1CCN(c2nc(OC[C@@H]3CCCN3CCCCNC(=O)OC(C)(C)C)nc3c2CCN(c2cccc4cccc(Cl)c24)C3)C[C@@H]1CC#N ; C=CC(=O)N1CCN(c2nc(OC[C@@H]3CCCN3CCCCN)nc3c2CCN(c2cccc4cccc(Cl)c24)C3)C[C@@H]1CC#N]

### 8.1 Preparation of Compound 10

A mixture of 2-[(2S]-4-[7-(8-chloro-1-naphthyl)-2-[[(2S)-pyrrolidin-2-yl]methoxy]-6,8-dihydro-5H-pyrido[3,4-]pyrimidin-4-yl]-1-prop-2-enoyl-piperazin-2-yl]acetonitrile (1.0 g, 1.75 mmol, 1.0 eq), tert-butyl N-(4-bromobutyl)carbamate (441 mg, 1.75 mmol, 358 μL, 1 eq), K₂CO₃ (725 mg, 5.24 mmol, 3.0 eq) and KI (290 mg, 1.75 mmol, 1 eq) in ACN (10 mL) was degassed and purged with N₂ 3 times. The reaction was stirred at 60° C. for 16 h under a N₂ atmosphere. The reaction mixture was filtered, concentrated under reduced pressure and purified by column chromatography (SiO₂. Petroleum ethcr:EtOAc=10/1 to 0/1) to give tert-butyl N-[4-[(2b)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]butyl]carbonate (1.2 g, 1.61 mmol, 92°i° yield) as a white solid: MS (ES) m/z: 743.6 (M+H)⁺, Rₜ=0.76 min.

### 8.2 Preparation of Compound Int. 3

To a solution of ten-butyl N-[4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4d]pyrimidin-2-yl]oxymethyl]pyrrolidin-1-yl]butyl]carbamate (1.0 g, 1.35 mmol, 1 eq) in DCM (10 mL) was added TFA (3 mL). The reaction was degassed and purged with N₂ 3 times, and stored at 20° C. for 1 h under a N₂ atmosphere. The reaction mixture was filtered, concentrated under reduced pressure and purified by prep-HPLC to give 2-[(2S)-4-[2-[[(2S)-1-(4-aminobutyl)pyrrolidin-2-yl]methoxyl-7-(8-chloro-1-naphthyl)-6, ti-dihydro-5H-pyrido[3,4-d]pyrimidin-4-yl]-1-prop-2-enoyl-piperazin-2-yl]acetonitrile (0.3 g, 466 μmol, 35% yield) as a white solid; MS (ESI) m/z: 043.5 (M+H)⁺, Rₜ=0.66 min.

### A4. Synthesis of Int. 4

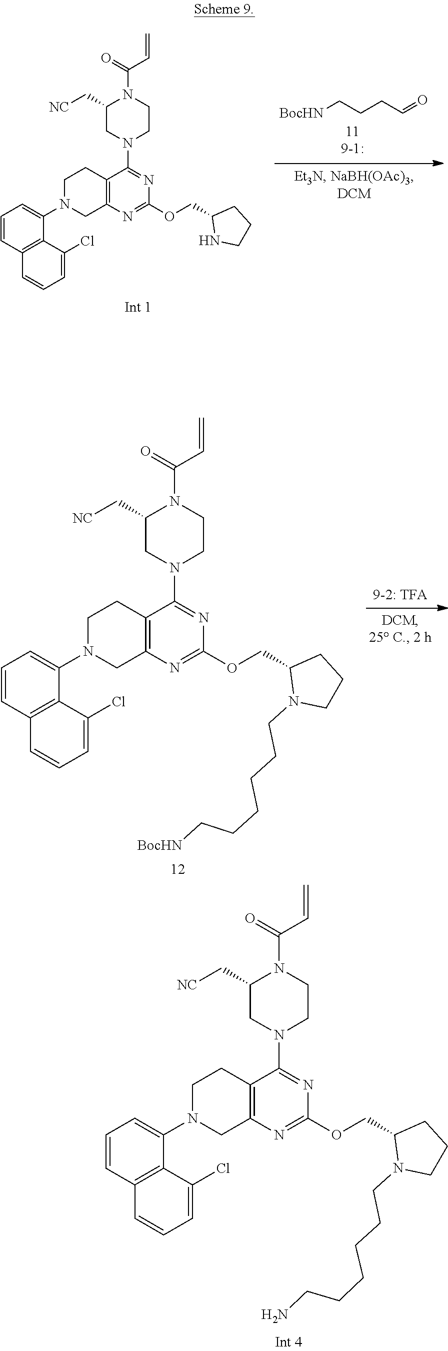 [SMILES: C=CC(=O)N1CCN(c2nc(OC[C@@H]3CCCN3)nc3c2CCN(c2cccc4cccc(Cl)c24)C3)C[C@@H]1CC#N ; CC(C)(C)OC(=O)NCCCC=O ; C=CC(=O)N1CCN(c2nc(OC[C@@H]3CCCN3CCCCCCNC(=O)OC(C)(C)C)nc3c2CCN(c2cccc4cccc(Cl)c24)C3)C[C@@H]1CC#N ; C=CC(=O)N1CCN(c2nc(OC[C@@H]3CCCN3CCCCCCN)nc3c2CCN(c2cccc4cccc(Cl)c24)C3)C[C@@H]1CC#N]

### 9 Preparation of Compound Int. 4

Intermediate 4 was synthesized following similar procedures as Scheme 7.

### A5. Synthesis of Int. 5

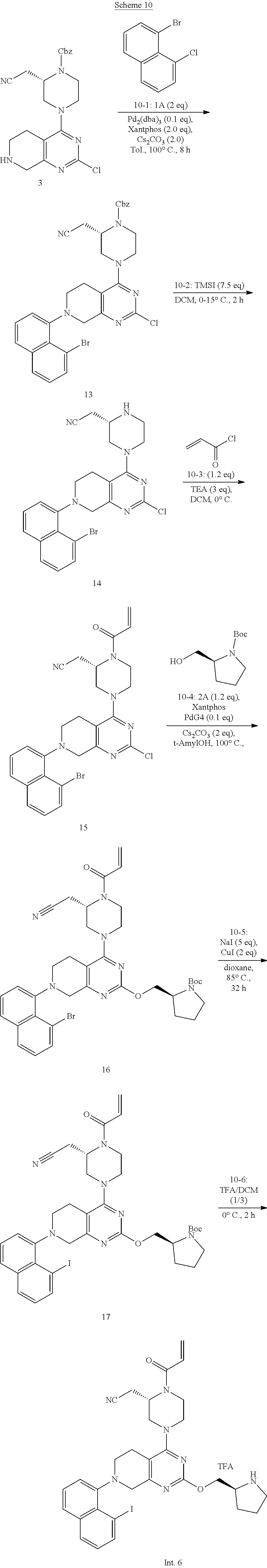

### 10.1 Preparation of Compound 13

A mixture of benzyl (2S)-4-(2-chloro-5,6,7,8-tetrahydropyrido[3,4-d]pyrimidin-4-yl)-2-(cyanomethyl)piperazine-1-carboxylate (29 g, 67.9 mmol, 1 eq), 1,8-dibromonaphthalene (38.9 g, 136 mmol, 2 eq), Xantphos (7.86 g, 13.6 mmol, 0.2 eq), Cs₂CO₃ (44.3 g, 136 mmol, 2 eq) and Pd₂(dba)₃ (6.22 g, 6.79 mmol, 0.1 eq) in PhMe (300 mL) was degassed and purged with N₂ 3 times, and the mixture stirred at 100° C. for 16 h under a N₂ atmosphere. The reaction mixture was filtered, diluted with a saturated NH₄Cl solution (300 mL) and extracted with EtOAc (200 mL×3). The combined organic layers were concentrated under reduced pressure and purified by column chromatography (SiO₂, petroleum ether:EtOAc=10:1 to 1:10) to give benzyl (2S)-4-[7-(8-bromo-1-naphthyl)-2-[[(2S)-1-tert-butoxycarbonylpyrrolidin-2-yl]methoxy]-6,8-dihydro-5H-pyrido[3,4-d]pyrimidin-4-yl]-2-(cyanomethyl)piperazine-1-carboxylate (40 g, 50.2 mmol, 74% yield) as a white solid: MS (ESI) m/z: 631.1 (M+H)⁺, Rₜ=0.99 min.

### 10.2 Preparation of Compound 14

A mixture of benzyl (2S)-4-[7-(8-bromo-1-naphthyl)-2-chloro-6,8-dihydro-5H-pyrido[3,4-d]pyrimidin-4-yl]-2-(cyanomethyl)piperazine-1-carboxylate (37.0 g, 58.5 mmol, 1 eq) and TMSI (87.9 g, 439 mmol, 59.8 mL, 7.5 eq) in CH₃CN (370 mL) was degassed and purged with N₂ for 3 times, and the mixture stirred at 20° C. for 5 h under a N₂ atmosphere. The reaction mixture was concentrated under reduced pressure and the residue purified by column chromatography (SiO₂, Petroleum ether:EtOAc=10:1 to 5:1) to give 2 [(2S)-4-[7-(8-bromo-1-naphthyl)-2-chloro-6,8 dihydro-5H-pyrido[3,4-d]pyrimidin-4-yl]piperazin-2-yl]acetonitrile (26 g, 52.2 mmol, 89% yield) as a yellow solid. MS (ESI) m/z: 497.2 (M+H)⁺, Rₜ=0.74 min.

### 10.3 Preparation of Compound 15

To a solution of 2-[(21)-4-[7-(8-bromo-1-naphthyl)-2-chloro-6,8-dihydro-5H-pyrido[3,4-d]pyrimidin-4-yl]piperazin-2-yl]acetonitrile (22 g, 44.2 mmol, 1 eq) in DCM (20 mL) was added TEA (13.4 g, 133 mmol, 18.5 mL, 3 coq) and prop-2-enoyl chloride (4.8 g, 53.0 mmol, 4.32 mL, 1.2 eq). The mixture was stirred at 0° C. for 0.5 h. The reaction mixture was concentrated under reduced pressure and the residue purified by column chromatography (SiO₂, petroleum ether:EtOAc=10/1 to 5/1) to give 2-[(2S)-4-[7-(8-bromo-1-naphthyl)-2-chloro-6,8-dihydro-5H-pyrido[3,4-d]pyrimidin-4-yl]-1-prop-2-enoyl-piperazin-2-yl]acetonitrile (9.0 g, 16.3 mmol, 37% yield) as a yellow solid. MS (ESI) m/z: 551.2 (M+H)⁺, Rₜ=0.89 min.

### 10.4 Preparation of Compound 16

A mixture of 2-[(2S)-4-[7-(8-bromo-1-naphthyl)-2-chloro-6,8dihydro-5H-pyrido[3,4-d]pyrimidin-4-yl]-1-prop-2-enoyl-pipet in-2-yl]acetonitrile (15.0 g, 27.2 mmol, 1 eq), tert-butyl (2S)-2-(hydroxymethyl)pyrrolidine-1-carboxylate (6.56 g, 32.6 mmol, 1.2 eq), XantPhosPd G4 (5.23 g, 5.43 mmol, 0.2 eq) and Cs₂CO₃ (17.7 g, 54.4 mmol, 2 eq) in t-Amy10H (150 mL) was degassed and purged with N₂ for 3 times. The reaction mixture was stirred at 1(a) ° C. for 2 h under a N₂ atmosphere. The mixture was filtered, diluted with water (50 mL), extracted with 3:1 DCM:isopropanol (120 mL), dried over Na₂SO₄, filtered and concentrated under reduced pressure. The residue was purified by prep-HPLC to give tert-butyl(2S)-2-[[7-(8-bromo-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidine-1-carboxylate (2.1 g, 293 μmol, 32% yield) as a white solid; ¹H NMR (CHLOROFORM-d): δ 7.75-7.86 (2H, m), 7.58-7.70 (1H, m), 7.46 (1H, q, J=7,9,12,3 Hz), 7.21-7.27(2H, m), 6.51-6.71 (1H, m), 6.40 (1H, br d, J=16.8 Hz), 5.82 (1H, br d, J=11.0 Hz), 4.89-5.25 (1H, m), 4.53-4.73 (1H, m), 4.29-4.49 (2H, m), 4.17-4.23 (1H, m), 3.97-4.11 (2H, m), 3.50-3.96 (4H, m), 2.98-3.45 (7H, m), 2.48-2.94 (3H, m), 1.76-2.01 (3H, m), 1.45 (9H, s).

### 10.5 Preparation of Compound 17

A mixture of tert-butyl (2S)-2-[[7-(8-bromo-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidine-1-carboxylate (0.30 g, 419 μmol, 1 eq), NaI (314 mg, 2.09 mmol, 5 eq), CuI (159 mg, 837 μmol, 2 eq) in dioxane (2 mL) was degassed and purged with N₂ for 3 times, and then the mixture was stirred at 85° C. for 72 h under a N₂ atmosphere. The reaction mixture was filtered and concentrated under reduced pressure and the residue purified by column chromatography (SiO₂, petroleum ether:EtOAc=10:1 to 5:1) to give tert-butyl (2S)-2 [[4-[(3 S)-3-(cyanomethyl)-4-prop-2-enoyl-piperazin-1-yl]-7-(8-iodo-1-naphthyl)-6,8-dihydro-5H-pyrido[3,4-d pyrimidin-2-yl]oxymethyl]pyrrolidine-1 carboxylate (0.20 g, 262 μmol, 63% yield) as a yellow solid; ¹H NMR (CHLOROFORM-d): δ 8.23 (1H, dd, J=1.6, 7.2 Hz), 7.84 (1H, br d, J=8.1 Hz), 7.64 (1H, br dd, J=4.9, 7.7 Hz), 7.37-7.58 (2H, m), 7.34 (1H, br d, J=7.4 Hz), 7.08 (1H, t, J=7.7 Hz), 6.51-6.71 (1H, m), 6.39 (111, br d, J=16.8 Hz), 5.82 (1H, br d, J=10.0 Hz), 4.92-5.15 (1H, m), 4.50-4.72 (1H, m), 4.27-4.43 (2H, m), 3.83-3.94 (1H, m), 3.05-3.71 (11H, m), 2.46-2.84 (3H, m), 1.80-1.99 (4H, m), 1.43 (9H, br s).

### 10.6 Preparation of Int. 5

A mixture of tert-butyl (2S)-2-[[4-[(3S)-3-(cyanomethyl)-4-prop-2-enoyl-piperazin-1-yl]-7-(8-iodo-1-naphthyl)-6,8-dihydro-5H-pyrido[3,4-d]pyrimidin-2-yl]oxymethyl]pyrrolidine-1-carboxylate (0.50 g, 650 μmol, 1 eq) in DCM (1 mL) was added TFA (1 ml), the reaction degassed and purged with N₂ 3 times, then stirred at 0° C. for 1 h under a N₂ atmosphere. The residue was purified by prep-HPLC to give 2-[(1S)-4-[7-(8-iodo-1-naphthyl)-2-[[(2S)-pyrrolidin-2-yl]methoxy]-6,8-dihydro-5H-pyrido[3,4-d]pyrimidin-4-yl]-1-prop-2-enoyl-piperazin-2-yl]acetonitrile (126 mg, 189 μmol, 48% yield) as a white solid. ¹H NMR (DMSO-d6) δ 9.12-9.26 (1H, m), 8.61-8.79 (1H, m), 8.22 (1H, d, J=7.3 Hz), 7, 1(1H, d, J=8.1 Hz), 7.77 (1H, dd, J=3.6, 7.7 Hz), 7.53-7.66 (111, m), 7.36-7.48 (111, m), 7.17 (1H, t, J=7.7 Hz), 6.65-6.99 (1H, m), 6.19 (1H, br d, J=16.6 Hz), 5.66-5.87 (1H, m), 4.21-4.68 (8H, m), 3.93-4.06 (5H, m), 3.12-3.44 (8H, m), 2.80-2.92 (1H, m), 2.06-2.16 (1H, m), 1.91 (2H, ddd, J=2.5 and 7.4, 15.4 Hz), 1.64-1.78(1H, m). MS (ES) m/z: 664.4 (M+H)⁺. Rₜ=0.72 min.

### A6. Synthesis of Compound Ex-1

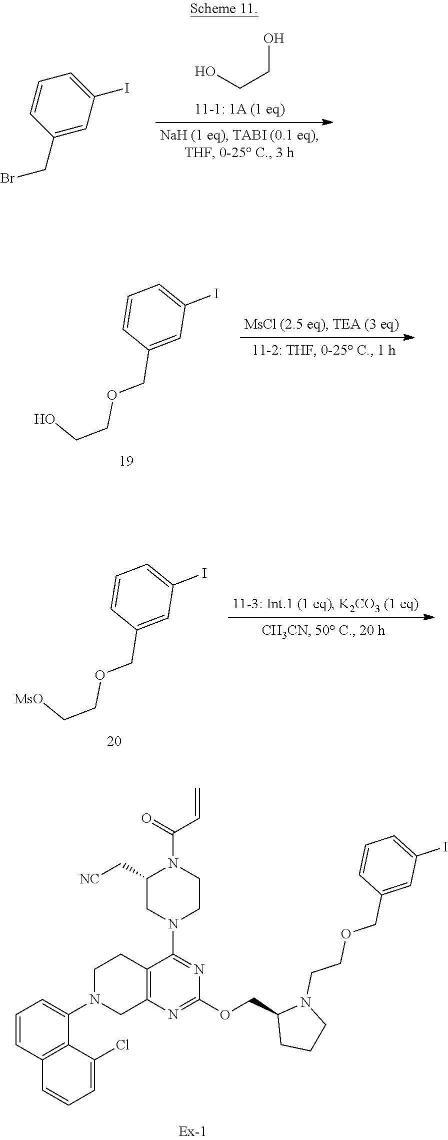

### 11.1 Preparation of Compound 19

To a solution of ethylene glycol (418 mg, 6.74 mmol, 377 μL, 2 eq) and TBAI (124 mg, 337 μmol, 0.1 eq) in THF (20 mL) was added 1-(bromomethyl)-3-iodo-benzene (1.0 g, 3.37 mmol, 1 eq) and the mixture was stirred for 20 minutes at 0° C. NaH (135 mg, 3.37 mmol, 60% purity, 1 eq) was added to the mixture at 0° C., and the reaction stirred at 25° C. for 3 h. The reaction mixture was quenched by a saturated solution of NH₄Cl (30 mL) and extracted with EtOAc (20 mL 3). The combined organic phase was dried over Na₂SO₄, filtered, and concentrated under reduced pressure. The residue was purified by prep-TLC (SiO₂, petroleum ether: EtOAc=1:1) to give 2-[(3-iodophenyl)methoxy]ethanol (0.38 g, 1.37 mmol, 41% yield) as a colorless oil; ¹H NMR (400 MHz, CHLOROFORM-d) δ ppm 3.59-3.64 (2H, m), 3.76-3.82 (2H, m), 4.52 (2H, s), 7.10 (1H, t, J=7.75 Hz), 7.31 (1H, d, J=7.63 Hz), 7.64 (1H, d, J=7.87 Hz), 7.72 (1H, s). MS (ESI) m/z: 278.0 (M+H)⁺. Rₜ=0.72 min.

### 11.2 Preparation of Compound 20

To a mixture of 2-[(3-iodophenyl)methoxy]ethanol 19 (150 mg, 539 μmol, 1 eq) and DIEA (209 mg, 1.62 mmol, 282 μL, 3 eq) in THE (4 mL) was added MsCl (154 mg, 1.35 mmol, 104 μL, 2.5 eq) at 0° C. The mixture was stirred at 25° C. for 1 h. The reaction mixture was quenched with a saturated NaHCO₃ solution (5 mL), extracted with dichloromethane (5 mL×3), the combined organic phase dried over Na₂SO₄, filtered and concentrated under reduced pressure to give crude 2-[(3-iodophenyl)methoxy]ethyl methanesulfonate (0.17 g) as a colorless oil; ¹H NMR (400 MHz, CHLOROFORM-d) δ ppm 3.06 (3H, s), 3.73-3.80 (2H, m), 4.36-4.46 (2H, m), 4.53 (2H, s), 7.10(1H, t. J=7.8 Hz), 7.30 (1H, br d, J=7.7 Hz), 7.65 (1H, d. J=7.8 Hz), 7.71 (1H, s). MS (ESI) mu: 356.0 (M+H)⁺. Rₜ=0.81 min.

### 11.3 Preparation of Compound Ex-1

To a mixture of 2-[(3-iodophenyl)methoxy]ethyl methanesulfonate 20 (0.17 g, 477 μmol, 1 eq) and 2-[(2S)-4-[7-(8-chloro-1-naphthyl)-2-[[(2S)-pyrrolidin-2-yl]methoxy]-6,8-dihydro-5H-pyrido[3,4-d]pyrimidin-yl]-1-prop-2-enoyl-piperazin-2-yl]acetonitrile (273 mg, 477 μmol, 1 eq) in CH₃CN (4 mL) was added K₂CO₃ (66 mg, 477 μmol, 1 eq) and the mixture stirred at 50° C. for 20 h. The reaction mixture was filtered and concentrated under reduced pressure. The residue was purified by prep-TLC (SiO₂, petroleum ether:EtOAc=1:1) to give 2-[(2S)-4-[7-(8-chloro-1-naphthyl)-2-[[(2S)-1-[2-[(3-iodophenyl)methoxy]ethyl]pyrrolidin-2-yl]methoxy]-6,8-dihydro-5H-pyrido[3,4-d]pyrimidin-4-yl]-1-prop-2-enoyl-piperazin-2-yl]acetonitrile (5.5 mg, 6.58 μmol, 1.4% yield) a white solid. ¹H NMR (40) MHz, CHLOROFORM-d) δ ppm 1.66-1.91(4H, m), 1.95-2.12(1H, m), 2.28-2.43 (1H, m), 2.56-2.73 (2H, m), 2.77-2.86(1H, m), 2.89-3.05 (2H, m), 3.08-3.33 (5H, m) 3.35-3.50(1H, m), 3.54-3.72 (3H, m), 3.77-3.97(2H, m), 4.01-4.19 (3H, m), 4.33-4.52(4H, m) 4.94-5.30(1H, m), 5.83(1H, br d, J=10.5 Hz), 6.34-6.47(1H, m), 6.50-6.67 (1H, m), 7.00-7.08(1H, m), 7.18-7.26 (2H, m), 7.34(1H, br t, J=7.8 Hz), 7.40-7.49(1H, m), 7.52 (1H, br d, J=6.2 Hz) 7.56-7, 65 (2H, m) 7.68 (1H, s) 7.76 (1H, br d, J=8.2 Hz). MS (ESI) m/z: 831.2 (M+H)⁺, Rₜ=2.54 min.

### A7. Synthesis of Compound Ex-2

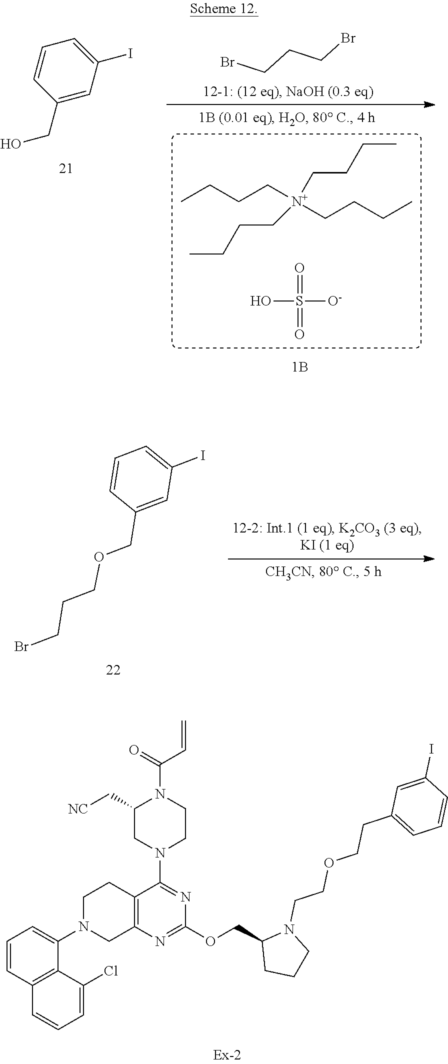 [SMILES: OCc1cccc(I)c1 ; BrCCCBr ; CCCC[N+](CCCC)(CCCC)CCCC ; O=S(=O)([O-])O ; BrCCCOCc1cccc(I)c1 ; C=CC(=O)N1CCN(c2nc(OC[C@@H]3CCCN3CCOCCc3cccc(I)c3)nc3c2CCN(c2cccc4cccc(Cl)c24)C3)C[C@@H]1CC#N]

### 12.1 Preparation of Compound 22

To a solution of (3-iodophenyl)methanol 21 (300 mg, 1.28 mmol, 1 eq) and 1,3-dibromopropane (3.11 g, 15.4 mmol, 1.57 mL, 12 eq) were added 50% NaOH aqueous solution (31 mg, 0.3 eq) and hydrogen sulfate; tetrabutylammonium (4.4 mg, 12.8 μmol, 0.01 eq). The mixture was stirred at 80° C. for 4 h. The reaction mixture was extracted with EtOAc (10 mL×3). The combined organic layers were washed with brine (10 mL×2), dried over Na₂SO₄, filtered and concentrated under reduced pressure. The residue was purified by column chromatography (SiO₂, petroleum ether:EtOAc=10:1) to give crude 1-(3-bromopropoxymethyl)-3-iodo-benzene 22 (400 mg) as a white oil: 1H NMR (400 MHz, CHLOROFORM-d) δ ppm 2.15 (1H, quin, J=6.16 Hz), 3.52-3.63 (2H, m), 4.04 (1H, dt, J=5.6. 1.31 Hz), 4.47 (2H, s), 5.17-5.40 (1H, m), 5.83-6.11 (1H, m), 7.09 (1H, td, J=7.8. 2.1 Hz), 7.31(1H, br t, J=6.3 Hz), 7.63 (1H, br d, J=7.8 Hz), 7.71 (1H, d, J=7.8 Hz).

### 12.2 Preparation of Compound Ex-2

A mixture of 1-(3-bromopropoxymethyl)-3-iodo-benzene 22 (149 tng, 210 μmol, 50% purity, 1.2 eq), 2 [(2S)-4-[7-(8 chloro-1-naphthyl)-2-[[(2S)-pyrrolidin-2-yl]methoxy]-6,8-dihydro-5H-pyrido[3,4-d]pyrimidin-4-yl]-1-prop-2-enoyl-piperazin-2-yl]acetonitrile (100 mg, 175 μmol, 1 eq). K₂CO₃ (72 mg, 524 μmol, 3 eq), KI (29 mg, 175 μmol, 1 eq) in CH₃CN (2 mL) was degassed and purged with N₂ 3 times. The mixture was stirred at 50° C. for 5 h under a N, atmosphere. The reaction mixture was filtered, concentrated under reduced pressure and purified by prep-TLC (SiO₂, EtOAc: MeOH=10:1) to give 2-[(2S)-4-[7-(8-chloro-1-naphthyl)-2-[[(2S)-1-[3-[(3-iodophenyl)methoxy]propyl]pyrrolidin-2-yl]methoxy]-6,8-dihydro-5H-pyrido[3,4-d]pyrimidin-4-yl]-1-prop-2-enoyl-piperazin-2-yl]acetonitrile (28 mg, 33 μmol, 19% yield) as a white solid; ¹H NMR ¹H NMR (400 MHz, CHLOROFORM-d) δ ppm 1.53 (2H, br s), 1.65-1.83(5H, m), 1.90-2.03(1H, m), 2.13-2.24(1H, m), 2.33-2.43(1H, m), 2.46-2.57 (1H, m), 2.62-2.75(1H, m), 2.80(1H, br s), 2.87-2.97 (2H, m), 3.00-3.13 (3H, m), 3.13-3.23(1H, m), 3.26-3.39(1H, m), 3.41-3.47 (2H, m), 3.48-3.55 (1H, m), 3.72 (1H, br d, J=17.6 Hz), 3.78-3.86 (1H, m), 3.94-4.03 (2H, m), 4.24-4.41 (4H, m), 4.82-5.15(1H, m), 5.74 (1H, br d, J=10. 3 Hz), 6.28-6.37(1H, m), 6.43-6.59(1H, m), 6.92-6.99(1H, m), 7.17(2H, br d, J=3.2 Hz), 7.26(1H, t, J=7.76 Hz), 7.36 (1H, dt, J=12.7, 7.8 Hz), 7.44 (1H, d, J=7.3 Hz), 7.49 (1H, d, J=8.0 Hz), 7.54 (1H, t, J=7.1 Hz), 7.58 (1H, s), 7.68 (1H, d, J=8.07 Hz).
MS (ES) m/z: 846.3.423.8(½ m/z). Rₜ=2.52 min.

### A8. Synthesis of Compound Ex-3

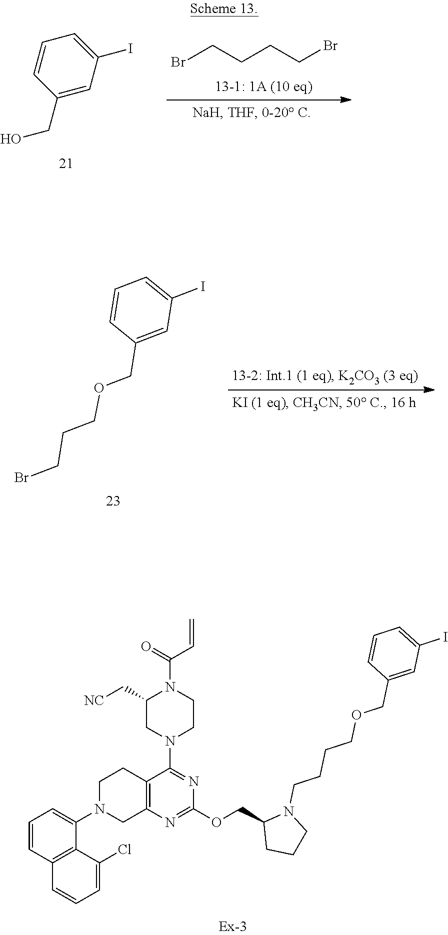 [SMILES: OCc1cccc(I)c1 ; BrCCCCBr ; BrCCCOCc1cccc(I)c1 ; C=CC(=O)N1CCN(c2nc(OC[C@@H]3CCCN3CCCCOCc3cccc(I)c3)nc3c2CCN(c2cccc4cccc(Cl)c24)C3)C[C@@H]1CC#N]

### 13.1 Preparation of Compound 23

To a solution of (3-iodophenyl)methanol 21 (1.0 g, 4.27 mmol, 1 eq) in DMF (5 mL) was slowly added NaH (427 mg, 10.7 mmol, 2.5 eq), the mixture stirred at 0° C. for 15 minute, 1,4-dibromobutane (9.23 g, 42.7 mmol, 10 eq) added at 0° C., and the mixture stirred at 15° C. for 3 h. The reaction mixture was quenched with a saturated NH₄Cl solution (20 mL) and extracted with EtOAc (20 mL×3). The combined organic phase was dried over Na₂SO₄, filtered, and concentrated under reduced pressure. The residue purified by column chromatography (petroleum ether: EtOAc=10:1 to 5:1) to give 1-(4-bromobutoxymethyl)-3-iodo-benzene (1.03 g, 2.79 mmol, 65% yield) as a colorless oil: ¹H NMR (400 MHz, CHLOROFORM-d) δ ppm 1.73-1.85 (2H, m), 1.94-2.02 (2H, m), 3.42-3.52(4H, m), 4.45 (2H, s), 7.09 (1H, t, J=7.75 Hz), 7.30 (1H, d, J=7.63 Hz), 7.63 (1H, d, J=7.88 Hz), 7.70 (1H, s). MS (ESI) m/z: 367.9 (M+H)⁺, Rₜ=0.89 min.

### 13.2 Preparation of Compound Ex-3

To a solution of 1-(4-bromobutoxymethyl)-3-iodo-benzene 23 (65 mg, 175 μmol, 1 eq), 2-[(2S)-4-[7-(8-chloro-1-naphthyl)-2-([(2S)-pyrrolidin-2-yl]methoxyl-6,8-dihydro-5H-pyrido[3,4-d]pyrimidin-4-yl]-1-prop-2-enoyl-piperazin-2-yl]acetonitrile (0.10 g, 175 μmol, 1 eq) in CH₃CN (3 mL) was added K₂CO₃ (72 mg, 524 μmol, 3 eq) and KI (29 mg, 175 μmol, 1 eq). The mixture was stirred at 50° C. for 16 h. The reaction mixture was filtered, concentrated under reduced pressure and purified by prep-HPLC to give 2 [(2S)-4-[748 chloro-1-naphthyl)-2-[[(2S)-1-[4-[(3-iodophenyl)methoxy]butyl]pyrrolidin-2-yl]methoxy]-6,8-dihydro-5H-pyrido[3,4-d]pyrimidin-4-yl]-1-prop-2-enoyl-piperazin-2-yl]acetonitrile (77 mg, 90 μmol, 51% yield) as a white solid; 1H NMR (400 MHz, CHLOROFORM-d) δ ppm 1.62 (3H, br d, J=6.0 Hz), 1.78 (4H, br s), 1.94-2.07(1H, m), 2.18-2.31(1H, m), 2.33-2.40 (1H, m), 2.53-2.65(1H, m), 2.68-2.94 (4H, m), 3.02 (1H, br dd, J=16.7, 8.34 Hz), 3.10-3.31 (4H, m), 3.38-3.52 (3H, m), 3.56-3.65 (1H, m), 3.76-3.95(2H, m), 3.99-4.18(3H, m), 4.31-4.44 (4H, m), 4.75-5.34(1H, m), 5.82 (1H, br d, J=10.4 Hz), 6.34-6.44 (1H, m), 6.50-6.65 (1H, m), 7.05 (1H, t, J=7.8 Hz), 7.17-7.26(2H, m), 7.33 (1H, t, J=7.8 Hz), 7.44 (1H, d t, J=13.59. 7.8 Hz), 7.52 (1H, br d, J=7.3 Hz), 7.57-7.64 (2H, m), 7.67 (1H, s), 7.75 (1H, br d, J=8.1 Hz). MS (ESI) m/z: 859.2(M/2+H), Rₜ=2.57 min.

### A9. Synthesis of Compound Ex-4

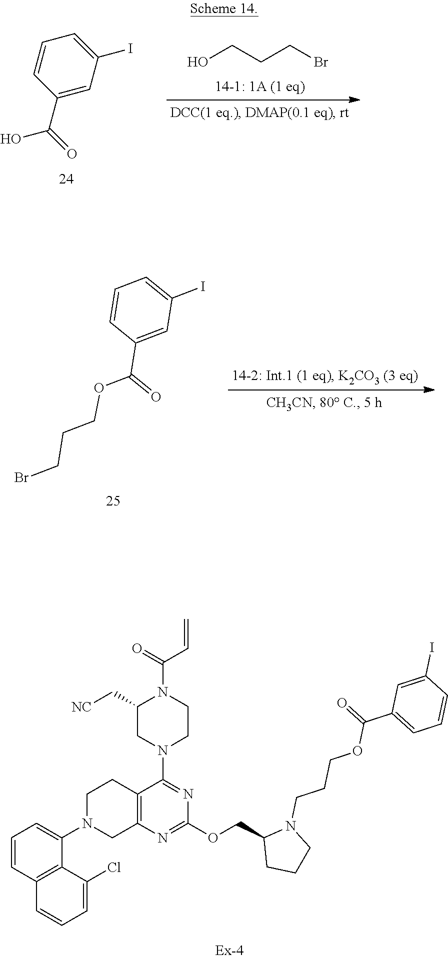 [SMILES: O=C(O)c1cccc(I)c1 ; OCCCBr ; O=C(OCCCBr)c1cccc(I)c1 ; C=CC(=O)N1CCN(c2nc(OC[C@@H]3CCCN3CCCOC(=O)c3cccc(I)c3)nc3c2CCN(c2cccc4cccc(Cl)c24)C3)C[C@@H]1CC#N]

### 14.1 Preparation of Compound 25

To a solution of 3-iodobenzoic acid 24 (0.50 g, 2.02 mmol, 1 eq) and 3-bromopropan-1-ol (560 mg, 4.03 mmol, 364 μL, 2 eq) in DCM (5 mL) was added CDI (360 mg, 2.22 mmol, 1.1 eq), DMAP (123 mg, 1.01 mmol, 0.5 eq) and Et₃N (306 mg, 3.02 mmol, 421 μL, 1.5 eq). The mixture was stirred at 15° C. for 16 h. The reaction mixture was quenched with a saturated NH₄Cl solution (10 mL) and extracted with EtOAc (10 mL×3). The combined organic phase was dried over Na₂SO₄, filtered, and concentrated under reduced pressure. The residue was purified by column chromatography (petroleum ether: EtOAc=10:1 to 5:1) to give 3-bromopropyl 3-iodobenzoate 25 (0.12 g, 330 μmol, 16% yield) as a white solid; ¹H NMR (400 MHz, CHLOROFORM-d) δ ppm 2.33 (2H, q, J=6.3 Hz), 3.55 (2H, t. J=6.5 Hz), 4.48 (2H, t, J=6.1 Hz), 7.20 (1H, t, J=7.9 Hz), 7.90 (1H, d, J=8.0 Hz), 8.01 (1H, d, J=7.8 Hz), 8.37 (1H, t, J=1.53 Hz). MS (ESI) m/z: 367.9 (M+H)⁺, Rₜ=0.94 min.

### 14.2 Preparation of Compound Ex-4

To a solution of 3-bromopropyl 3-iodobenzoate 25 (64.5 mg, 175 μmol, 1 eq), 2-[(2S)-4-[7-(8-chloro-1-naphthyl)-2-[[(2S)-pyrolidin-2-yl]methoxy]-6,8-dihydro-5H-pyrido[3,4-d]pyrimidin-4-yl]-1-prop-2-enoyl-piperazin-2-yl]acetonitrile (0.10 g, 175 μmol, 1 eq) in CH₃CN (2 mL) was added KI (29 mg, 175 μmol, 1 eq) and K₂CO₃ (72 mg, 524 μmol, 3 eq). The mixture was stirred at 50° C. for 16 h. The reaction mixture was filtered, concentrated under reduced pressure and purified by prep-HPLC to give 3-[(2S)-2 [[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]propyl]3-iodobenzoate (36 mg, 42.2 μmol, 24% yield) as a white solid: ¹H NMR (400 MHz, CHLOROFORM-d) δ ppm 1.78(3H, br s) 1.96-2.06(3H, m), 2.22-2.31 (1H, m), 2.51-2.62 (2H, m), 2.69-2.88(2H, m), 2.91 (1H, br s), 3.00-3.19 (5H, m), 3.20-3.29(1H, m), 3.32-3.50(1H, m), 3.58(1H, br d, J=10.5 Hz), 3.75-3.86 (1H, m), 3.88-4.19(4H, m), 4.29-4.43 (4H, m), 4.91-5.22(1H, m), 5.83 (1H, br d, J=9.9 Hz), 6.34-6.47(1H, m), 6.52-6.71 (1H, m), 7.11 (1H, br t, J=7.8 Hz), 7.17-7.25 (1H, m), 7.34(1H, br t, J=7.8 Hz), 7.44 (1H, dt, J=12.99. 7.8 Hz), 7.52 (1H, br d, J=7.5 Hz), 7.61 (1H, br t. J=7.5 Hz), 7.76 (1H, br d, J=8.0 Hz), 7.81 (1H, br d, J=7.7 Hz), 7.95 (1H, br d, J=7.2 Hz), 8.33 (1H, s). MS (ESI) m/z: 859.2 (M+H)⁴. Rₜ=0.85 min.

### A10. Synthesis of Compound Ex-5

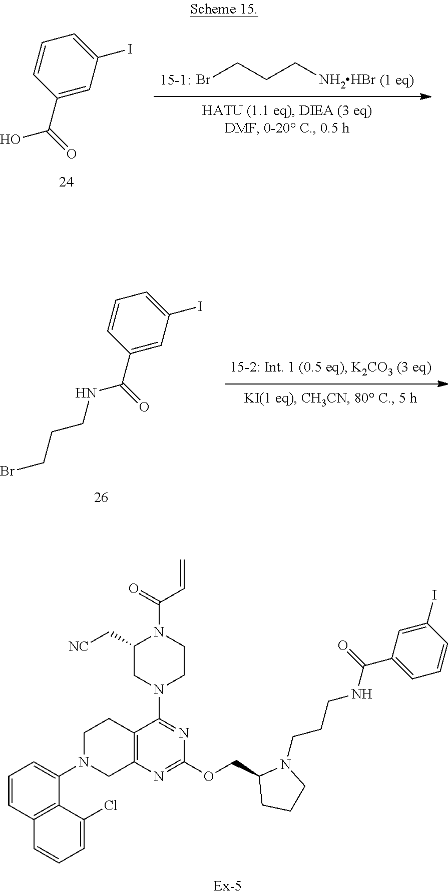

### 15.1 Preparation of Compound 26

To a solution of 3-iodobenzoic acid 24 (1.0 g, 4.03 mmol, 1.0 eq) in DMF (20 mL) was added HATU (1.69 g, 4.44 mmol, 1.1 eq) and DIEA (1.04 g, 8.06 mmol, 1.40 mL, 2 eq) at 0° C. The mixture was stirred at 0° C. for 1 h, 3-bromopropan-1-amine hydrobromide (1.03 g, 4.03 mmol, 1 eq. HCl) added at 0° C., the reaction mixture diluted with H₂O (40 mL) and extracted with EtOAc (20 mL×2). The combined organic layers were washed with H₂O (40 mL×2), dried over Na₂SO₄, filtered and concentrated under reduced pressure. The residue was purified by column chromatography (SiO₂, petroleum ether:EtOAc=10:1 to 3:1) to give N-(3-bromopropyl)-3-iodo-benzamide (0.70 g, 1.90 mmol, 47% yield) as a white solid: ¹H NMR (400 MHz, CHLOROFORM-d) δ ppm 2.21 (2H, quip, J=6.48 Hz), 3.50 (2H, t, J=6.4 Hz), 3.62 (2H, q, J=6.5 Hz), 6.39 (1H, br s), 7.18 (1H, t. J=7.83 Hz), 7.72 (1H, dt, J=7.8, 1.3 Hz), 7.84 (1H, dt, J=7.8, 1.3 Hz), 8.11 (1H, t, J=1.7 Hz). MS (ESI) m/z: 368.0 (M+H)⁺. Rₜ=0.80 min.

### 15.2 Preparation of Compound Ex-5

A mixture of 2-[(2S)-4-[7-(8-chloro-1-naphthyl)-2-[[(2S)-pyrrolidin-2-yl]methoxy]-6,8-dihydro-5H-pyrido[3,4-d pyrimidin-4-yl]-1-prop-2-enoyl-piperazin-2-yl]acetonitrile (100 mg, 175 μmol, 1 eq), N-(3-bromopropyl)-3-iodo-benzamide (64 mg, 175 μmol, 1 eq). K₂CO₃ (72 mg, 524 μmol, 3 eq). KI (29 mg, 175 μmol, 1 eq) in CH₃CN (2 mL) was degassed and purged with N₂ for 3 times, and the mixture stored at 80° C. for 2 h under a N₂ atmosphere. The reaction mixture was filtered, concentrated under reduced pressure and the residue purified by prep-HPLC to give N-[3-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]propyl]-3-iodo-benzamide (35 mg, 40 μmol, 23% yield) as a white solid ¹H NMR (400 MHz, CHLOROFORM-d) δ ppm 1.71-1.95 (5H, m), 1.99-2.12 (1H, m), 2.20-2.41 (1H, m), 2.44-2.57 (1H, m), 2.59-2.75 (2H, m), 2.76-3.00((5H, m), 3.11-3.35(5H, m), 3.53-3.65(2H, m), 3.67-4.07 (4H, m), 4.17-4.39 (3H, m), 4.78-5.15 (1H, m), 5.83 (1H, br d, J=10.5 Hz), 6.32-6.43 (1H, m), 6.47-6.67 (1H, m), 7.07 (1H, q. J=7.5 Hz), 7.15-7.23 (1H, m), 7.34 (1H, t, J=7.8 Hz), 7.43-7.56 (2H, m), 7.60-7.72 (2H, m), 7.73-7.89(2H, m), 8.11 (1H, br s), 9.04 (1H, br s). MS (ESI) m/z: 859.1 (M+H)⁺, Rₜ=2.44 min.

### A11. Synthesis of Ex-6

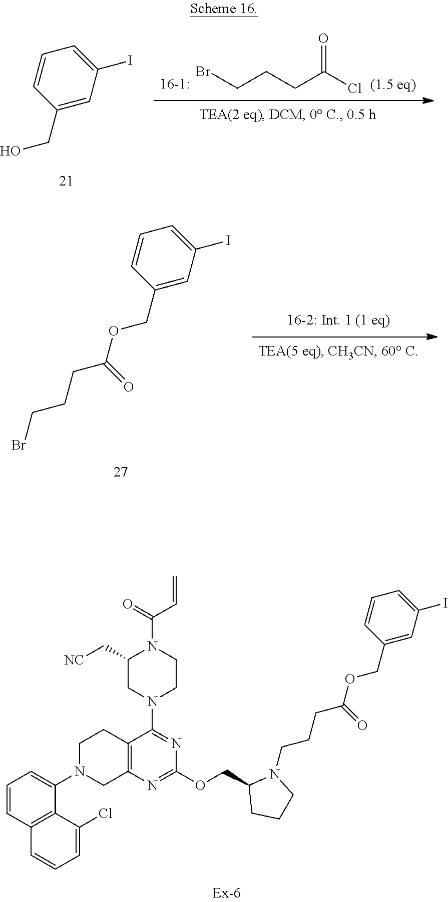 [SMILES: OCc1cccc(I)c1 ; O=C(Cl)CCCBr ; O=C(CCCBr)OCc1cccc(I)c1 ; C=CC(=O)N1CCN(c2nc(OC[C@@H]3CCCN3CCCC(=O)OCc3cccc(I)c3)nc3c2CCN(c2cccc4cccc(Cl)c24)C3)C[C@@H]1CC#N]

### 16.1 Preparation of Compound 27

A mixture of (3-iodophenyl)methanol (0.20 g, 855 μmol, 1 eq), 4-bromobutanoyl chloride (190 mg, 1.03 mmol, 119 μL, 1.2 eq) and TEA (173 mg, 1.71 mmol, 238 μL, 2 eq) in DCM (5 mL) was degassed and purged with N₂ 3 times, and the mixture stirred at 0° C. for 4 h under a N₂ atmosphere. The reaction mixture was quenched with a saturated NH₄Cl solution (5 mL) and then extracted with EtOAc (5 mL×4). The combined organic phase was dried over Na₂SO₄, filtered and concentrated under reduced pressure. The residue was purified by prep-TLC (SiO₂, PE: EA=3:1) to give (3-iodophenyl)methyl 4-bromobutanoate (0.27 g, 705 μmol, 82% yield) as a white oil: 1H NMR (400 MHz, CHLOROFORM-d) δ ppm 2.21 (2H, quin. J=6.8 Hz), 2.58 (2H, t, J=7.2 Hz) 3.48 (2H, t, J=6.4 Hz), 5.07 (2H, s), 7.11 (1H, t, J=7.8 Hz), 7.32 (1H, d, J=7.6 Hz), 7.67 (1H, d, J=8.0 Hz), 7.72 (1H, s). MS (ESI) m/z: 381.9 (M+H)⁺, Rₜ=0.94 min.

### 16.2 Preparation of Compound Ex-6

A mixture of (3-iodophenyl)methyl 4-bromobutanoate (100 mg, 261 μmol, 5 eq), 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-prop-2-enoyl-piperazin-2-yl]acetonitrile (30 mg, 52 μmol, 1 eq) and TEA (26 mg, 261 μmol, 36 μL, 5 eq) in CH₃CN (2 mL) was degassed and purged with N₂ 3 times. The mixture was stirred at 50° C. for 16 h. The reaction mixture was filtered and concentrated under reduced pressure. The residue was purified by prep-HPLC to give (3-iodophenyl)methyl 4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]butanoate (13.0 mg, 14.9 μmol, 29% yield) as a white solid. ¹H NMR (4011 MHz, CHLOROFORM-δ ppm 1.60-1.91 (7H, m), 2.01 (1H, br s), 2.23 (1H, br s) 2.34-2.50(3H, m), 2.60(1H, br d, J=13.6 Hz), 2.76-2.93(3H, m), 2.97-3.31 (5H, m), 3.35-3.66 (2H, m), 3.74-3.93 (2H, m), 3.96-4.18 (3H, m), 4.20-4.52 (2H, m), 5.01 (2H, br d, J=4.1 Hz), 5.82 (1H, br d, J=10.5 Hz), 6.39 (1H, br d, J=16.8 Hz), 6.59 (1H, br s), 7.06 (1H, br t, J=7.7 Hz), 7.18-7.25 (1H, m), 7.29 (1H, br s), 7.33 (1H, br t. J=7.8 Hz), 7.44 (1H, dt, J=13.4, 7.7 Hz), 7.52 (1H, br d, J=7.5 Hz), 7.59-7.65 (2H, m), 7.68 (1H, s), 7.75 (1H, br d, J=8.0 Hz). MS (ESI) m/z: 873.2 (M+H)⁺, Rₜ=3.87 min.

### A12. Synthesis of Compound Ex-7

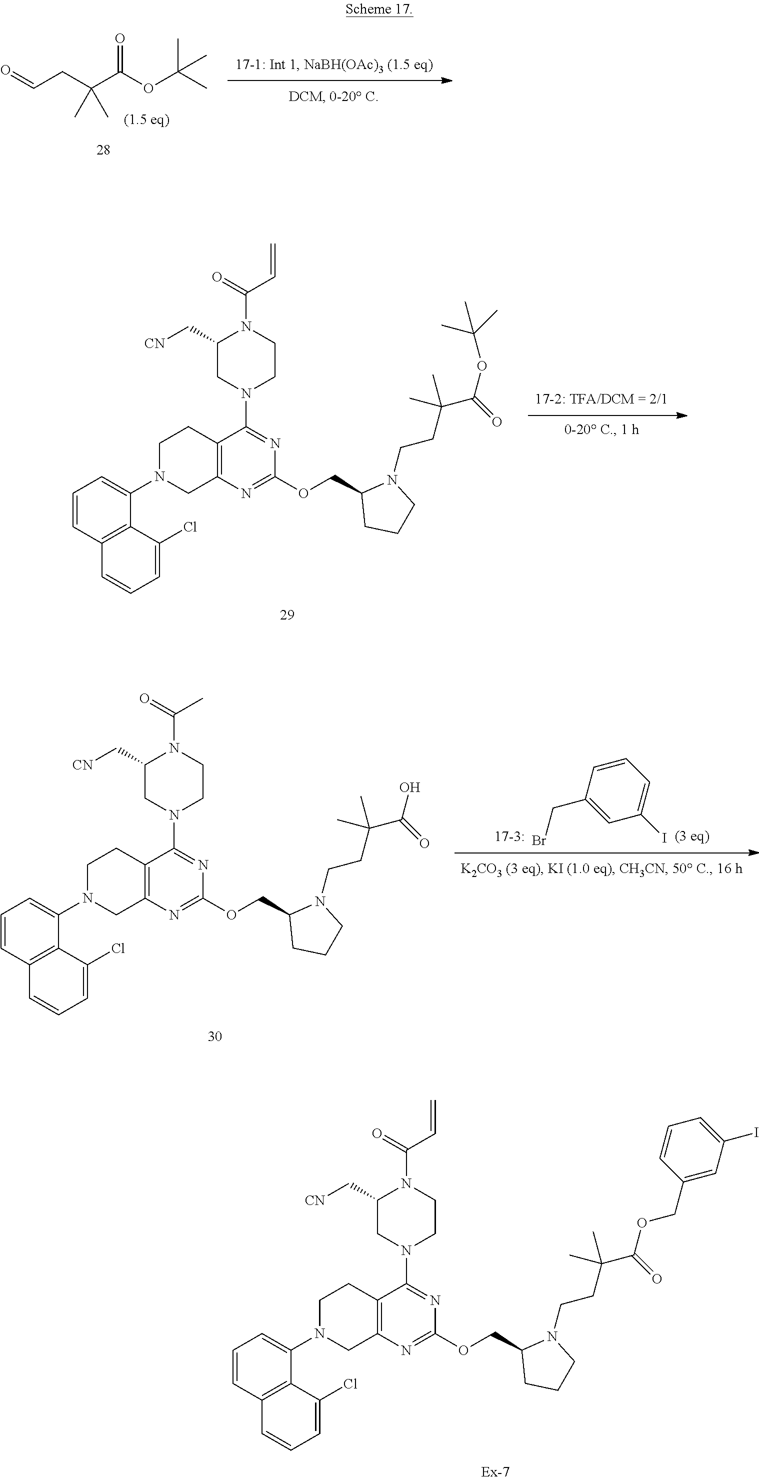

### 17.1 Preparation of Compound 29

A mixture of tert-butyl 2,2-dimethyl-4-oxo-butanoate (85.5 mg, 459 μmol, 1.5 eq), 2-[(2S)-4-[748-chloro-1-naphthyl)-2-[[(2S)-pyrrolidin-2-yl]methoxy]-6,8-dihydro-5H-pyrido[3,4-d]pyrimidin-4-yl]-1-prop-2-enoyl-piperazin-2-yl]acetonitrile (175 mg, 306 μmol, 1 eq). NaBH(OAc)₃ (97 mg, 459 μmol, 1.5 eq) in DCM (1 mL) was degassed and purged with N, for 3 times, and stirred at 25° C. for 2 h under a N, atmosphere. The reaction mixture was quenched by addition DCM (2 mL) at 25° C., and concentrated under reduced pressure. The residue was purified by prep-TLC (SiO₂, EtOAc:MeOH=5:1) to give tert-butyl 4 [(2S)-2-[[7-(8-chloro-1-naphthyl)-4-[(3 S)-3-(cyanomethyl)-4-prop-2-enoyl-piperazin-1-yl]-6,8-dihydro-5H-pyrido[3,4-d]pyrimidin-2-yl]oxymethyl]pyrrolidin-1 yl]-2,2-dimethyl-butanoate (60 mg, 81 μmol, 26% yield) as a white solid. 1H NMR (400 MHz, CHLOROFORM-d) δ ppm 1.10-1.15 (6H, m), 1.18 (2H, s), 1.42 (9H, d, J=2.1 Hz), 1.45 (4H, s) 1.80 (4H, br d, J=6.9 Hz), 2.02-2.05 (1H, m), 2.13-2.42 (2H, m), 2.55-2.65 (1H, m), 2.77-2.93 (3H, m), 3.00-3.24(4H, m), 3.56-3.64 (1H, m), 3.71 (1H, t, J=6.7 Hz), 3.78-3.86 (1H, m), 3.99-4.10(2H, m), 4.33-4.48 (2H, m), 5.83(1H, br d, J=11.1 Hz), 6.40(1H, br d, J=17.0Ha), 6.55-6.72(1H, m), 7.18-7.26(1H, m), 7.34(1H, t, J=7.8Ha), 7.45(1H, dt, J=12.7, 7.8 Hz), 7.51-7.54 (1H, m), 7.62 (1H, t, J=7.3 Hz) 7.74-7.78 (1H, m). MS (ES) m/z: 742.4 (M+H)⁺, Rₜ=0.85 min.

### 17.2 Preparation of Compound 30

A mixture of tert-butyl 4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]-2,2-dimethyl-butanoate (20 mg, 26.9 μmol, 1 eq), TFA (770 mg, 6.75 mmol, 0.5 mL, 251 eq), in DCM (0.5 mL) was degassed and purged with N₂ for 3 times at 0° C., and the mixture was stored at 20° C. for 1 h under N₂ atmosphere. The reaction was concentrated under reduced pressure to give crude 4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]-2,2-dimethyl-butanoic acid (20 mg) as a yellow oil. MS (ESI) m/z: 686.6 (M+H)⁺, Rₜ=0.74 min.

### 17.3 Preparation of Compound Ex 7

A mixture of 4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]-2,2-dimethyl-butanoic acid (100 mg, 146 μmol, 1 eq), 1-(bromomethyl)-3-iodo-benzene (130 mg, 437 μmol, 3 eq). K₂CO₃ (60 mg, 437 μmol, 3 eq). KI (24 mg, 146 μmol, 1 eq) in DMF (2 mL) was degassed and purged with N₂ 3 times. The reaction was stirred at 40° C. for 16 h under a N₂ atmosphere and concentrated under reduced pressure. The residue was diluted with H₂O (3 mL), extracted with EtOAc (50 mL) and purified by prep-HPLC to give (3-iodophenyl)methyl4-[(2S)-2-[[(7-(8 chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]-2,2-dimethyl-butanoate (8 mg, 8.87 μmol, 6.1% yield) as a yellow solid. ¹H NMR (400 MHz, CHLOROFORM-d) δ ppm 1.23-1.27 (6H, m), 1.99-2.16(4H, m), 2.18-2.33 (2H, m), 2.58-2.77 (2H, m), 2.85-3.00(3H, m), 3.04-3.11 (1H, m), 3.13-3.20(2H, m), 3.51-3.60 (3H, m), 3.67-3.78 (2H, m), 3.88(1H, dt, J=11.5.5.9 Hz), 3.94-4.11 (2H, m), 4.18-4.29 (1H, m), 4.41-4.56(2H, m), 4.69-4.78 (1H, m), 4.89-5.12 (4H, m), 5.85(1H, br d, J=9.8 Hz), 6.35-6.46 (1H, m), 6.51-6.67(1H, m), 7.07-7.14 (1H, m), 7.21-7.25 (1H, m), 7.30-7.32(1H, m), 7.35 (1H, t. J=7.8 Hz), 7.46 (1H, td, J=7.8, 5.3 Hz), 7.52-7.56 (1H, m), 7.63-7.70 (3H, m), 7.77 (1H, d, J=8.3 Hz). MS (ESI) m/z: 902.5 (M+H)⁺, Rₜ=0.88 min.

### A13. Synthesis of Compound Ex-8

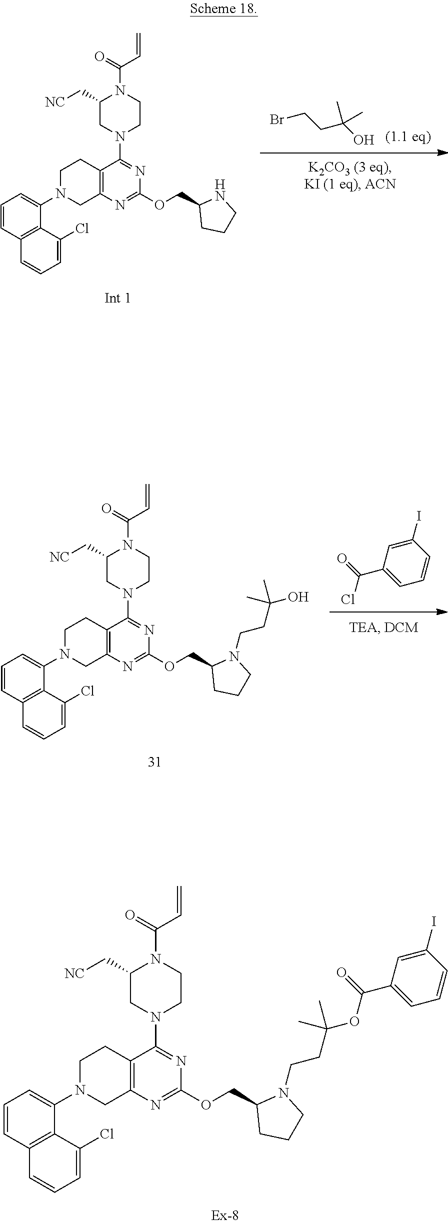

### 18.1 Preparation of Compound 31

To a solution of 2-[(2S)-4-[7-(8chloro-1-naphthyl)-2-[[(2S)-pyrrolidin-2-yl]methoxy]-6,8-dihydro-5H-pyrido[3,4-d]pyrimidin-4-yl]-1-prop-2-enoyl-piperazin-2-yl]acetonitrile (0.05 g, 87.4 μmol, 1.0 eq) in MeCN (5 mL) was added KI(14.5 mg, 87.4 μmol, 1 eq), K₂CO₃ (36 mg, 262 μmol, 3 eq) and 4-bromo-2-methyl-butan-2-ol (21.9 mg, 131 μmol, 1.5 eq). The mixture was stirred at 60° C. for 16 h. The reaction mixture was concentrated under reduced pressure and the residue purified by prep-TLC (SiO₂, EtOAc:methanol=10:1) to give 2-[(2S-4-[7-(8-chloro-1-naphthyl)-2-[[(2S)-1-(3-hydroxy-3-methyl-butyl)pyrrolidin-2-yl]methoxy]-6,8-dihydro-5H-pyrido[3,4-d]pyrimidin-4-yl]-1-prop-2-enoyl-piperazin-2-yl]acetonitrile (50 mg, 76 μmol, 87% yield) as a white solid: MS (ESI) m/z: 657.3 (M+H)⁺, Rₜ=0.73 min.

### 18.2 Preparation of Compound Ex-8

To a solution of 2-[(2S)-4-[7-(8-chloro-1-naphthyl)-2-[[(2S)-1-(3-hydroxy-3-methyl-butyl)pyrrolidin-2-yl]methoxy]-6,8-dihydro-5H-pyrido[3,4d]pyrimidin-4-yl]-1-prop-2-enoyl-piperazin-2-yl]acetonitrile (45 mg, 68.4 μmol, 1 eq) in DCM (5 mL) was added TEA (13.8 mg, 137 μmol, 19.0 μL, 2 eq) and 3-iodobenzoyl chloride (36 mg, 137 μmol, 2 eq). The mixture was stirred at 15° C. for 16 h. The reaction mixture was filtered and concentrated under reduced pressure. The residue was purified by prep-TLC (SiO₂. EtOAc: Methanol=10:1) to give [3-[(2S)-2-[[7-(8-chloro-1-naphthyl)-4-[(3S)-3-(cyanomethyl)-4-prop-2-enoyl-piperazin-1-yl]-6,8-dihydro-5Hpyrido[3,4-d]pyrimidin-2-yl]oxymethyl]pyrrolidin-1-yl]-1,1-dimethyl-propyl]3-iodobenzoate (20 mg, 23 μmol, 33% yield) as a white solid; ¹H NMR (400 MHz, CHLOROFORM-d) δ ppm 1.58 (6H, br s), 1.77 (3H, br s), 1.95-2.06 (1H, m), 2.08-2.21 (2H, m), 2.24-2.31 (1H, m), 2.47-2.55 (1H, m), 2.57-2.64 (1H, m), 2.78-2.85 (1H, m), 2.87-2.93 (1H, m), 2.97-3.32 (7H, m), 3.34-3.48 (1H, m), 3.59(1H, br dd, J=11.0, 4.1 Hz), 3.76-3.92(2H, m), 4.04-4.12 (2H, m), 4.30-4.42 (2H, m), 4.49-4.73 (1H, m), 4.91-5.20(1H, m), 5.83(1H, br d, J=10.3 Hz), 6.34-6.46(1H, m), 6.53-6.69(1H, m), 7.10(1H, t, J=7.9 Hz), 7.16-7.25 (1H, m), 7.32-7.37(1H, m), 7.42-7.48(1H, m), 7.52 (1H, d, J=6.7 Hz), 7.59-7.65 (1H, m), 7.74-7.82 (2H, m), 7.90(1H, d, J=7.9 Hz), 8.28 (1H, s). MS (ESI) m/z: 887.2 (M+H)⁺, Rₜ=2.65 min.

### A14. Synthesis of Compound Ex-9

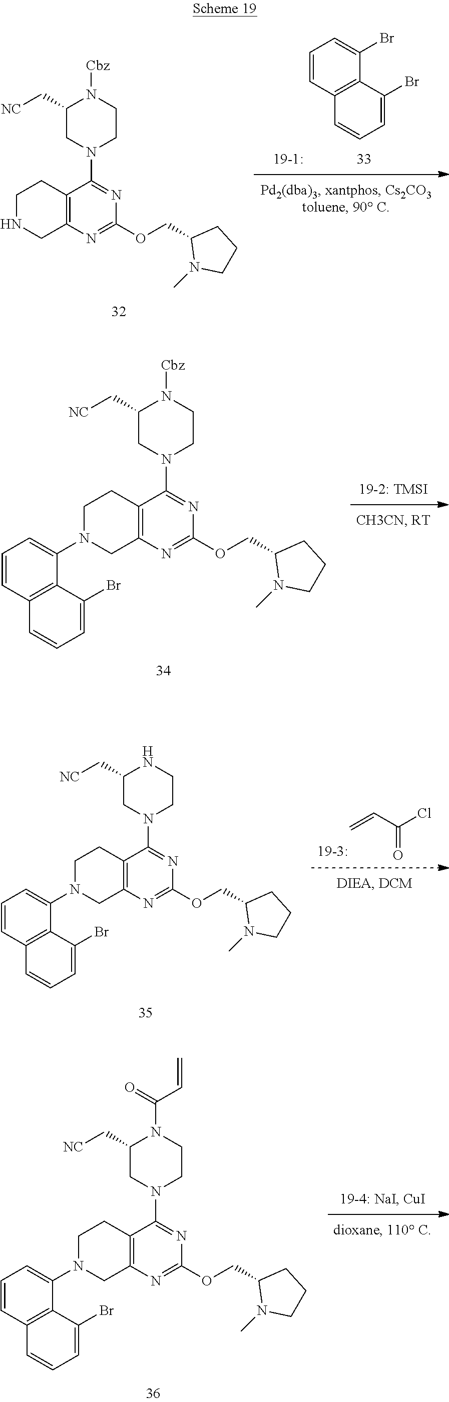

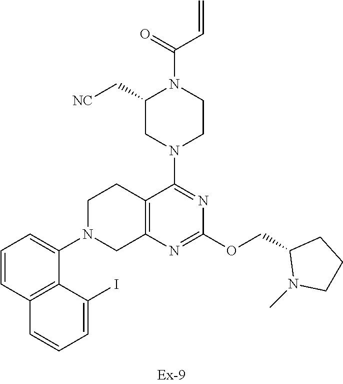 [SMILES: C=CC(=O)N1CCN(c2nc(OC[C@@H]3CCCN3C)nc3c2CCN(c2cccc4cccc(I)c24)C3)C[C@@H]1CC#N]

### 19.1 Preparation of Compound 34

Benzyl (2S)-2-(cyanomethyl)-4-[2-[[(2S)-1-methylpyrrolidin-2-yl]methoxy]-5,6,7,8-tetrahydropyrido[3,4-d]pyrimidin-4-yl]piperazine-1-carboxylate (0.80 g, 1.58 mmol), 1,8-dibromonaphthalene (905 mg, 3.16 mmol). Cs₂CO₃ (1.60 g, 4.91 mmol), Xantphos (200 mg, 346 μmol) and Pd₂(dba)₃ (160 mg, 175 μmol) in toluene (10 mL) was degassed and purged with N₂ 3 times. The mixture was stirred at 100° C. for 6 h under a N₂ atmosphere. The reaction mixture was filtered and the filtrate concentrated under reduced pressure. The residue was purified by column chromatography (SiO₂, petroleum ether:EtOAc=1:1 to DCM:methanol=10:1) to give benzyl (2S)-4-[7-(8-bromo-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carboxylate (0.50 g, 44% yield) as a brown solid. MS (ESI) m/z: 712.3 (M+2H)⁺, Rₜ=0.64 min.

### 19.2 Preparation of Compound 35

To a solution of benzyl (2S)-4-[7-(8-bromo-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazine-1-carboxylate (100 mg, 141 μmol) in CH₃CN (4 mL) was added TMSI (282 mg, 1.41 mmol) at 0° C. The mixture was stirred at 25° C. for 2 h. The reaction mixture was concentrated under reduced pressure and the residue purified by prep-TLC (SiO₂, DCM:MeOH=10:1) to give 2-[(2S)-4-[748-bromo-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (60 mg, 74% yield) as a brown solid; MS (ESI) m/z: 578.2 (M+2H)⁺, Rₜ=0.42 min.

### 19.3 Preparation of Compound 36

To a solution of 2-[(2S)-4-[7-(8-bromo-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (60 mg, 104 μmol) in CH₂C₁₂ (2 mL) was added TEA (32 mg, 312 μmol) and prop-2-enoyl chloride (19 mg, 208 μmol). The mixture was stirred at 25° C. for 1 h. The reaction mixture was concentrated under reduced pressure and the residue purified by prep-HPLC to give 2-[(2S)-4-[7-(8-bromo-1-naphthyl)-2-[[(2S)-1-methylpyrrolidin-2-yl]methoxy]-6,8-dihydro-5H-pyrido[3,4-d]pyrimidin-4-yl -1-prop-2-enoyl-piperazin-2-yl]acetonitrile (20 mg, 30% yield) as a white solid; ¹H NMR (400 MHz. CD₃OD): δ (ppm) 1.72-2.27 (5H, m), 2.63-2.80 (5H, m), 2.86-2.97 (1H, m), 3.11-3.26 (4H, m), 3.47-3.87 (4H, m), 4.05-4.37 (4H, m), 4.39-4.69 (4H, m), 5.85 (1H, d, J=10.4 Hz), 6.31 (1H, d, J=17.2 Hz), 6.73-7.01 (1H, m), 7.30 (1H, t, J=7.6 Hz), 7.38 (1H, d, J=12.4), 7.48-7.58 (1H, m), 7.72 (1H, d, J=8.4 Hz), 7.82 (1H, d, J=7.2 Hz), 7.89 (1H, d, J=8.0 Hz). MS (ESI) m/z: 632.2 (M+H)⁺, Rₜ=0.56 min.

### 19.4 Preparation of Compound Ex-9

A mixture of 2-[(2S)-4-[7-(8-bromo-1-naphthyl)-2-[[(2S)-1-methylpyrrolidin-2-yl]methoxy]-6,8-dihydro-5H-pyrido[3,4-d]pyrimidin-4-yl]-1-prop-2-enoyl-piperazin-2-yl]acetonitrile (10 mg, 15.9 μmol), NaI (6.0 mg, 40.0 μmol), CuI (2.00 mg, 10.5 μmol) in dioxane (2 mL) was degassed and purged with N₂ 3 times. The reaction mixture was stirred at 100° C. for 4 h under N₂ atmosphere, filtered and the filtrate concentrated under reduced pressure. The residue was purified by prep-HPLC to give 2-[(2S)-4-[7-(8-iodo-1-naphthyl)-2-[[(2S)-1-methylpyrrolidin-2-yl]methoxy]-6,8-dihydro-5H-pyrido[3,4-d]pyrimidin-4-yl]-1-prop-2-enoyl-piperazin-2-yl]acetonitrile (3.5 mg, 33% yield) as a white solid; ¹H NMR (400 MHz, CD₃OD): δ (ppm) 2.03-2.28 (3H, m), 2.35-2.55 (1H, m), 2.67-3.04 (3H, m), 3.05-3.11 (3H, m), 3.13-3.29 (2H, m), 3.40-3.85 (7H, m), 3.87-4.04 (1H, m), 4.74 (5H, s), 4.92-5.15 (2H, m), 5.87 (1H, d, J=9.6 Hz), 6.32 (1H, d, J=16.0 Hz), 6.75-6.98 (111, m), 7.09-7.18(1H, m), 7.42-7.51(1H, m), 7.52-7.61 (1H, m), 7.75 (1H, d, J=8.0 Hz), 7.94 (1H, d, J=7.6 Hz), 8.27 (1H, d, J=7.2 Hz). MS (ESI) m/z: 678.3(M/2+H)⁺, Rₜ=0.57 min.

### A15. Synthesis of Compound Ex-10

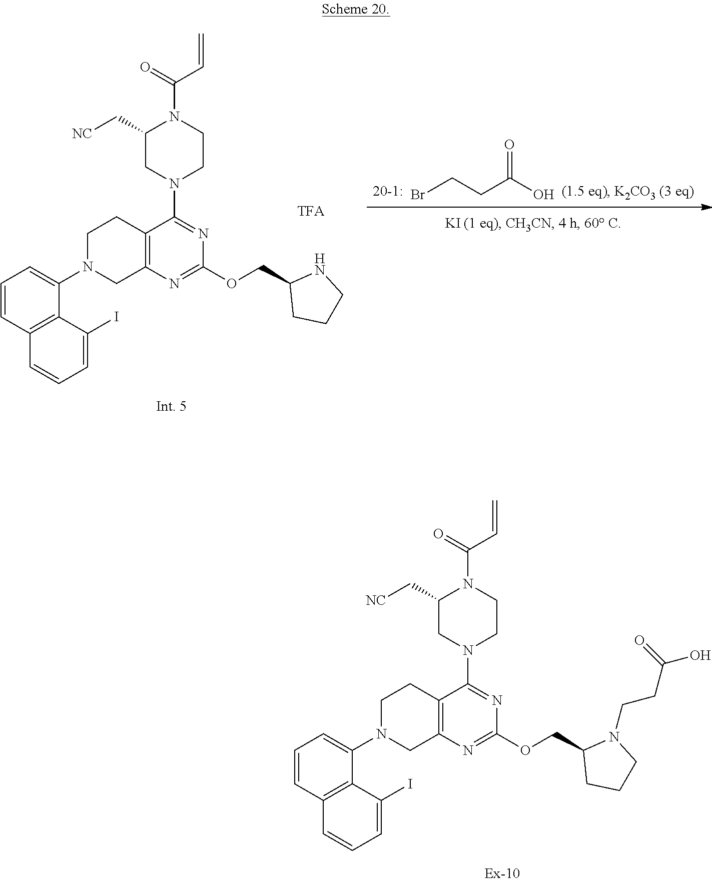 [SMILES: C=CC(=O)N1CCN(c2nc(OC[C@@H]3CCCN3)nc3c2CCN(c2cccc4cccc(I)c24)C3)C[C@@H]1CC#N ; O=C(O)CCBr ; C=CC(=O)N1CCN(c2nc(OC[C@@H]3CCCN3CCC(=O)O)nc3c2CCN(c2cccc4cccc(I)c24)C3)C[C@@H]1CC#N]

### 20.1 Preparation of Compound Ex-10

A mixture of 2-[(2S)-4-[7-(8-iodo-1-naphthyl)-2-[[(2S)-pyrrolidin-2-yl]methoxyl-6,8-dihydro-5H-pyrido[3,4-d]pyrimidin-4-yl]-1-prop-2-enoyl-piperazin-2-yl]acetonitrile (70 mg, 105 μmol, 1 eq), 3-bromopropanoic acid (48 mg, 316 μmol, 33 μL, 3 eq). K₂CO₃ (44 mg, 316 μmol, 3 eq) and KI (18 mg, 105 μmol, 1 eq) in CH₃CN (1 mL) was degassed and purged with N₂ 3 times. The mixture was stirred at 60° C. for 16 h. The reaction mixture was concentrated under reduced pressure and the residue purified by prep-HPLC to give 3-[(2S]-2-[[4-[(3.5-3-(cyanomethyl)-4-prop-2-enoyl-piperazin-1-yl]-7-(8-iodo-1-naphthyl)-6,8-dihydro-5H-pyrido[3,4-d]pyrimidin-2-yl]oxymethyl]pyrrolidin-1-yl]propanoic acid (18.0 mg, 27.2 μmol, 26% yield) as a white solid; ¹H NMR (400 MHz, DMSO-d₆) δ ppm 1.77-2.10 (3H, m) 2.14-2.27 (1H, m) 2.64-2.85 (3H, m) 2.86-3.21 (6H, m) 3.60-3.72 (4H, m) 3.85-4.17(6H, m) 4.34-4.59(3H, m)4.6-1-5.07(2H, m)5.78(1H, br d, J=11.2 Hz) 6.19 (1H, br d, J=16.7 Hz) 6.74-6.96 (1H, m) 7.17 (1H, t, J=7.69 Hz)7.37-7.47(1H, m) 7.57(1H, q, J=8.3 Hz) 7.76(1H, dd, J=8.1, 3.5 Hz) 7.99 (1H, d, J=8.1 Hz) 8.22 (1H, d, J=7.2 Hz) 9.35-9.67 (1H, m). MS (ESI) m/z: 735.2 (M+H)⁺, Rₜ=2.26 min.

### A16. Synthesis of Ex-11

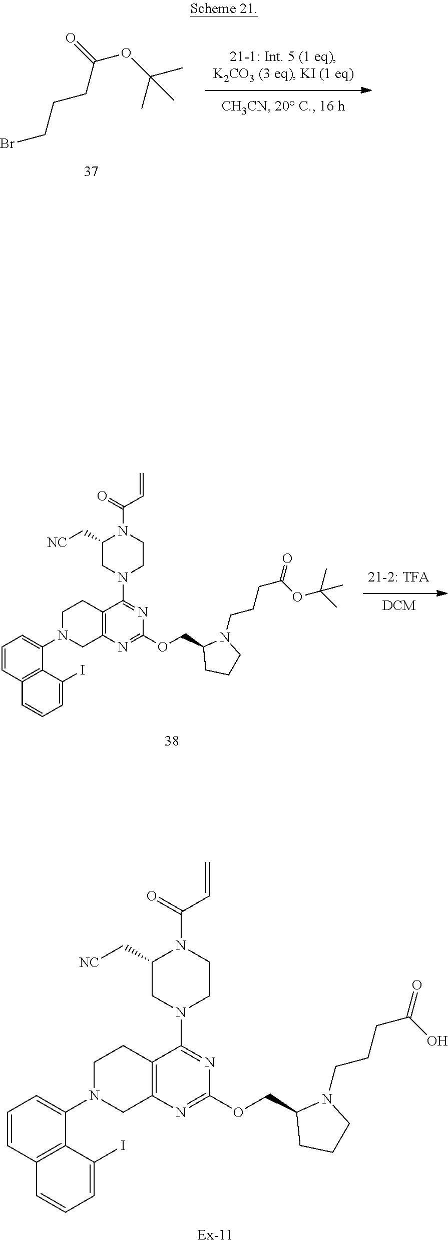

### 21.1 Preparation of Compound 38

A mixture of 2-[(2S)-4-[7-(8-iodo-1-naphthyl)-2-[[(2S)-pyrrolidin-2-yl]methoxy]-6,8-dihydro-5H-pyrido[3,4-d pyrimidin-4-yl]-1-prop-2-enoyl-piperazin-2-yl]acetonitrile (50 mg, 75.4 μmol, 1 eq) and tert-butyl 4-bromobutanoate (134 mg, 603 μmol, 8 eq) in CH₃CN (2 mL) was added KI (13 mg, 75 μmol, 1 eq) and K₂CO₃ (31 mg, 226 μmol, 3 eq). The mixture was stirred at 20° C. for 16 h under a N₂ atmosphere. The reaction was concentrated under reduced pressure, to give crude tert-butyl 4-[(2S)-2-[[4-[(3S)-3-(cyanomethyl)-4-prop-2-enoyl-piperazin-1-yl]-7-(8-iodo-1-naphthyl)-6,8-dihydro-5H-pyrido[3,4-d]pyrimidin-2-yl]oxymethyl]pyrolidin-1-yl]butanoate (60 mg, 50 μmol, 66% yield) as a white solid.

### Preparation of Compound Ex-11

A mixture of crude tert-butyl 4-[(2S)-2-[[(4-[(3S)-3-(cyanomethyl)-4-prop-2-enoyl-piperazin-1-yl]-7-(8-iodo-1-naphthyl)-6,8-dihydro-5H-pyrido[3,4-d]pyrimidin-2-yl]oxymethyl]pyrolidin-1-yl]butanoate (50 mg, 6.21 μmol, 1 eq) in TFA (1 mL) and DCM (3 mL) was stirred at 20° C. for 3 h under a N₂ atmosphere. The reaction mixture was concentrated under reduced pressure and the residue purified by prep-TLC (SiO₂, EtOAc:MeOH=5:1) to give 4-[(2S)-2-[[4-[(3S)-3-(cyanomethyl)-4-prop-2-enoyl-piperazin-1-yl]-7-(8-iodo-1-naphthyl)-6,8-dihydro-5H-pyrido(3,4-d]pyrimidin-2-yl]oxymethyl]pyrrolidin-1-yl]butanoic acid (18.4 mg, 19.6 μmol, 36% yield) as a white solid: ¹H NMR (400 MHz, CHLOROFORM-d) δ ppm 1.76 (2H, br dd, J=12.8, 6.3 Hz), 1.84-1.99 (3H, m), 2.12-2.21(1H, m), 2.52-2.67 (4H, m), 2.87 (4H, br d, J=12.8 Hz), 3.02-3.26 (4H, m), 3.33-3.55 (4H, m), 3.60-3.85 (2H, m), 3.92-4.13 (2H, m), 4.19-4.38 (2H, m), 4.40-4.72 (2H, m), 4.93-5.18(1H, m), 5.82(1H, br d, J=10.4Ha), 6.39(1H, br d, 1=16.5 Hz), 6.49-6.69 (1H, m), 7.07 (1H, t, J=7.69 Hz), 7.28-7.37(1H, m), 7.43-7.55 (1H, m), 7.61-7.69 (1H, m), 7.84 (1H, br d, J=8.0 Hz), 8.23 (1H, dd, J=6.9. 3.75 Hz). MS (ESI) m/z: 749.2 (M+H)⁺, Rₜ=2.29 min.

### A17. Synthesis of Ex-12

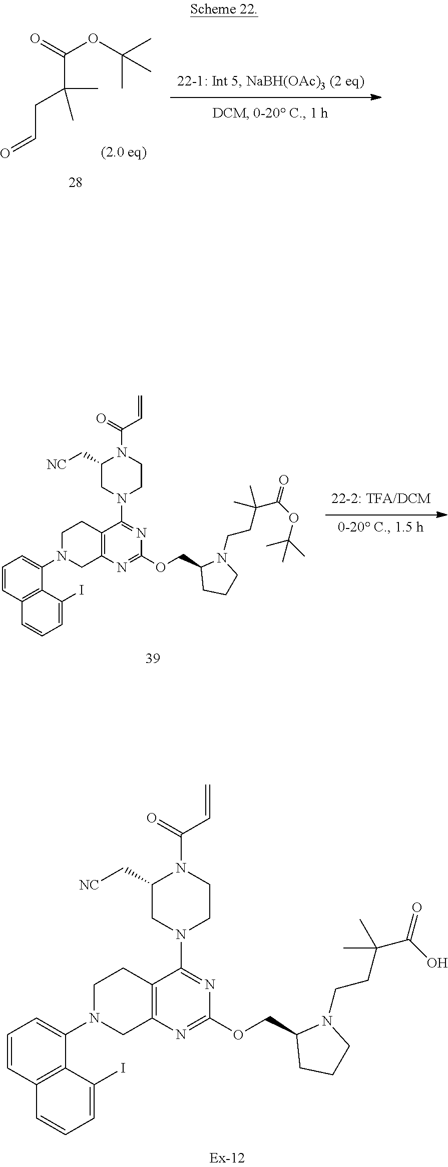 [SMILES: CC(C)(C)OC(=O)C(C)(C)CC=O ; C=CC(=O)N1CCN(c2nc(OC[C@@H]3CCCN3CCC(C)(C)C(=O)OC(C)(C)C)nc3c2CCN(c2cccc4cccc(I)c24)C3)C[C@@H]1CC#N ; "0-20° C., 1.5 h" ; C=CC(=O)N1CCN(c2nc(OC[C@@H]3CCCN3CCC(C)(C)C(=O)O)nc3c2CCN(c2cccc4cccc(I)c24)C3)C[C@@H]1CC#N]

### 22.1 Preparation of Compound 39

To a solution of 2-[(2S)-4-[7-(8-iodo-1-naphthyl)-2-[[(2S)-pyrrolidin-2-yl]methoxy]-6,8-dihydro-5H-pyrido[3,4-d]pyrimidin-4-yl]-1-prop-2-enoyl-piperazin-2-yl]acetonitrile (80 mg, 121 μmol, 1 eq) and ter-butyl 2,2-dimethyl-4-oxo-butanoate (22.5 mg, 121 μmol, 1 eq) in DCM (1 mL) was added NaBH(OAc); (77 mg, 362 μmol, 3 eq) and TEA (37 mg, 362 μmol, 50 μL, 3 eq) at 0° C. The mixture was stirred at 20° C. for 1 h. diluted with DCM (10 mL), washed with water (50 mL), brine (30 mL), dried over Na₂SO₄, filtered, and concentrated under reduced pressure. The residue was purified by prep-TLC (SiO₂, EtoAc:MeOH=10:1) to give tert-butyl 4-[(2S)-2-[[4-[(3S)-3-(cyanomethyl)-4-prop-2-enoyl-piperazin-1-yl]-7-(8-iodo-1-naphthyl)-6,8-dihydro-5H-pyrido[3,4d]pyrindin-2-yl]oxymethyl]pyrrolidin-1-yl]-2,2-dimethyl-butanoate (60 mg, 72 μmol, 60% yield) as a white solid; MS (ESI) m/z: 833.3, Rₜ=0.83 min.

### 22.2 Preparation of Compound Ex-12

To a solution of tert-butyl 4-[(2S)-2-[[4-[(3S)-3-(cyanomethyl)-4-prop-2-enoyl-piperazin-1-yl]-7-(8-iodo-1-naphthyl)-6,8-dihydro-5H-pyrido[3,4-d]pyrimidin-2-yl]oxymethyl]pyrrolidin-1-yl]-2,2-dimethyl-butanoate (30 mg, 36 μmol, 1 eq) in DCM (2 mL) was added TFA (1 mL). The mixture was stirred at 20° C, for 2 h. The residue was purified directly by prep-HPLC to give 4-[(2S)-2-[[4-[(3S)-3-(cyanomethyl)-4-pop-2-enoyl-piperazin-1-yl]-7-(8-iodo-1-naphthyl)-6,8-dihydro-5H-pyrido[3,4-d]pyrimidin-2-yl]oxymethyl]pyrrolidin-1-yl]-2,2-dimethyl-butanoic acid (23 mg, 29.6 μmol, 41% yield) as a white solid: ¹H NMR (400 MHz, DMSO-d6) δ=9.59 (1H, br s), 8.22 (1H, d, J=7.3 Hz), 7.99 (1H, d, J=8.1 Hz), 7.77(1H, dd, J=3.7, 8.0 Hz), 7.63-7.53(1H, m), 7.47-7.38(1H, m), 7.17(1H, t, J=7.7 Hz), 6.95-6.75 (1H, m), 6.19 (1H, br d, J=16.5 Hz), 5.84-5.75 (1H, m), 5.01-4.70(1H, m), 4.59-4.50(1H, m), 4.40 (1H, br dd, J=7.2, 12.1 Hz), 4.14-3.90 (6H, m), 3.44-3.26 (4H, m), 3.20-3.05(5H, m), 2.95-2.82 (1H, m), 3.03-2.81 (1H, m), 2.78-2.62(1H, m), 2.60-2.51 (1H, m), 2.27-2.14(1H, m), 2.07-1.98 (1H, m), 1.95-1.75 (4H, m), 1.13-1.06 (6H, m). MS (ESI) m/z: 777.2, Rₜ=2.42 min.

### A18. Synthesis of Compound Ex-13

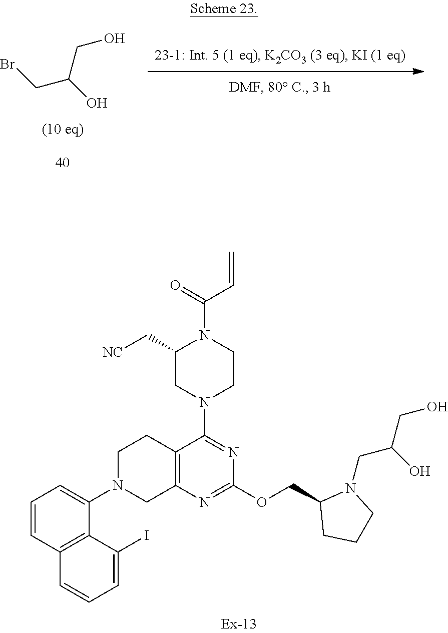 [SMILES: OCC(O)CBr ; C=CC(=O)N1CCN(c2nc(OC[C@@H]3CCCN3CC(O)CO)nc3c2CCN(c2cccc4cccc(I)c24)C3)C[C@@H]1CC#N]

### 23.1 Preparation of Compound Ex-13

A mixture of 2-[(2.5)-4-[7-(8-iodo-1-naphthyl)-2-[[(2S)-pyrrolidin-2-yl]methoxy]-6,8-dihydro-5H-pyrido[3,4-d]pyrimidin-4-yl]-1-prop-2-enoyl-piperazin-2-yl]acetonitrile (30 mg, 45.2 μmol, 1 eq), 3-bromopropane-1,2-diol (Ill mg, 452 μmol, 40 μL, 10 eq). KI (7.5 mg, 45.2 μmol, 1 eq) and K₂CO₃ (19 mg, 136 μmol, 3 eq) in DMF (2 mL) was degassed and purged with N₂ for 3 times. The reaction mixture was stirred at 80° C. for 16 h under a N₂ atmosphere, concentrated under reduced pressure and the residue purified by prep-HPLC to give 2-[(2S)-4-[2-[[)2S)-1-(2,3-dihydroxypropyl)pyrrolidin-2-yl]methoxy]-7-(8-iodo-1-naphthyl)-6,8-dihydro-5H-pyrido[3,4d]pyrimidin-4-yl]-1-prop-2-enoyl-piperazin-2-yl]acetonitrile (0.02 g, 27.1 μmol, 60% yield) as a white solid; ¹H NMR (400 MHz, DMSO-d6) δ ppm 1.74-1.96 (2H, m), 1.99-2.10(1H, m), 2.15-2.28 (1H, m), 2.62-2.76 (1H, m), 2.81-2.92(1H, m), 2.93-3.17 (4H, m), 3.18-3.32 (5H, m), 3.33-3.51 (5H, m), 3.97-4.18 (5H, m), 4.33-4.61 (3H, m), 4.64-5.10(2H, m), 5.79(1H, bid, J=10.9 Hz), 6.19(1H, br d, J=16.7 Hz), 6.77-6.95(1H, m) 7.17(1H, t, J=7.7 Hz), 7.36-7.47 (1H, m), 7.57 (1H, q, J=8.3 Hz), 7.77(1H, dd, J=8.1, 3.8 Hz), 7.99 (1H, d, J=8.1 Hz), 8.22 (1H, d, J=7.2 Hz), 9.28-9.60 (1H, m). MS (ESI) m/z: 737.2 (M+H)⁺, Rₜ=2.21 min.

### A19. Synthesis of Ex-14

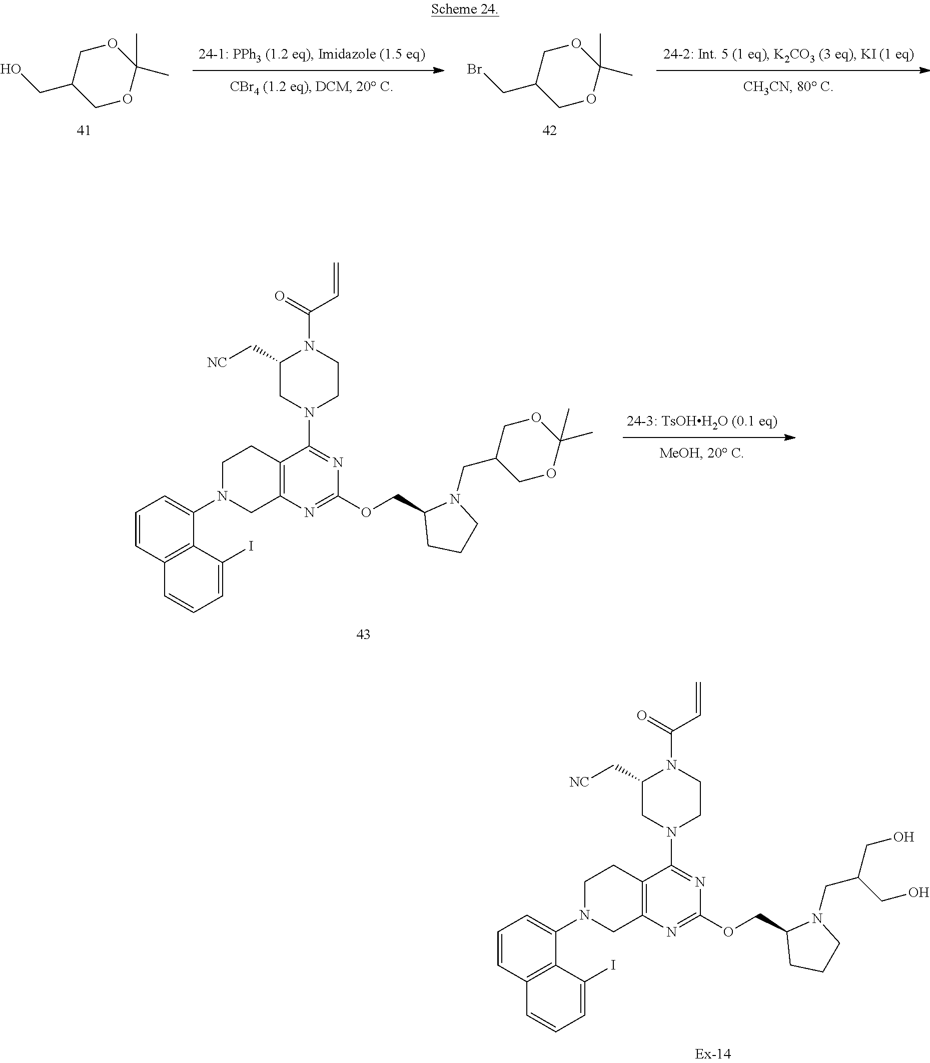

### 24.1 Preparation of Compound 42

A mixture of (2,2-dimethyl-1,3-dioxan-5-yl)methanol (0.30 g, 2.05 mmol, 1 eq). PPh₃ (645 mg, 2.46 mmol, 1.2 eq). CBr₄ (816 mg, 2.46 mmol, 1.20 eq) and imidazole (210 mg, 3.08 mmol, 1.50 eq) in DCM (3 mL), was stirred at 20° C. for 4 h under a N₂ atmosphere. The reaction mixture was concentrated under reduced pressure. The residue was purified by prep-TLC (SiO₂, petroleum ether:EtOAc=5:1) to give 5-(bromomethyl)-2,2-dimethyl-1,3-dioxane (0.30 g, 1.43 mmol, 70% yield) as a yellow oil: ¹H NMR (400 MHz, CHLOROFORM-d) δ ppm 1.43 (6H, d, J=12.2 Hz), 1.88-2.10 (1H, m), 3.51 (2H, d, J=7.0 Hz), 3.72-3.85 (2H, m), 4.05 (2H, dd, J=12.2.4.1 Hz).

### 24.2 Preparation of Compound 43

A mixture of 5-(bromomethyl)-2,2-dimethyl-1,3-dioxane (25 mg, 120 μmol, 5 eq), 2-[(2S)-4-[7-(8-iodo-1-naphthyl)-2-[[(2,5)-pyrrolidin-2-yl]methoxy]-6,8-dihydro-5H-pyrido[3,4-d]pyrimidin-4-yl]-1-prop-2-enoyl-piperazin-2-yl]acetonitrile (16 mg, 24 μmol, 1 eq), K₂CO₃ (9.9 mg, 72 μmol, 3 eq) and KI (4 mg, 24 μmol, 1 eq) in CH₃CN (2 mL) was stirred at 80° C. for 16 h under a N₂ atmosphere. The reaction mixture was filtered and concentrated. The residue was purified by prep-TLC (SiO₂, EtOAc:MeOH=5:1) to give 2-[(2S)-4-[2-[[(2S)-1-[(2,2-dimethyl-1,3-dioxan-5-yl)methyl]pyrrolidin-2-yl]methoxy]-7-(8-iodo-1-naphthyl)-6,8-dihydro-5H-pyrido[3,4d]pyrimidin-4-yl]-1-prop-2-enoyl-piperazin-2-yl]acetonitrile as a white solid. ¹H NMR (400 MHz, CHLOROFORM-d) δ ppm 1.38 (6 h. dd, J=9.23, 5.4 Hz), 1.69-1.83 (3H, m), 1.91-2.01 (2H, m), 2.18-2.26 (1H, m), 2.28-2.35(1H, m), 2.51-2.66 (1H, m) 2.76-2.94 (4H, m), 2.97-3.17 (3H, m), 3.18-3.30 (2H, m), 3.59 (1H, ddd, J=11.52, 7.6.4.0 Hz), 3.64-3.71 (2H, m), 3.78-3.84 (1H, m), 3.85-4.09 (7H, m), 4.26-4.44(2H, m), 4.96-5.22 (1H, m), 5.82 (1H, br d. J=10.3 Hz), 6.34-6.47 (1H, m), 6.54-6.69 (1H, m), 7.08 (1H, t. J=7.7 Hz), 7.34 (1H, d, J=6.9 Hz), 7.44-7.55 (1H, m), 7.64 (1H, t. J=7.5 Hz), 7.79-7.89 (1H, m), 8.23 (1H, d, J=6.6 Hz). MS (ESI) m/z: 791.2 (M+H)⁺, Rₜ=0.79 min.

### 24.3 Preparation of Compound Ex-14

A mixture of 2-[(2S)-4-[2-[[(2S)-1-[(2,2-dimethyl-1,3-dioxan-5-yl)methyl]pyrrolidin-2-yl]methoxyl-7-(8-iodo-1-naphthyl)-6,8-dihydro-5H-pyrido[3,4-d]pyrimidin-4-yl]-1-prop-2-enoyl-piperazin-2-yl]acetonitrile (50 mg, 63 μmol, 1 eq) and TsOH·H₂O (1.20 mg, 6.32 μmol, 0.1 eq) in MeOH (1 mL) was stirred at 20° C. for 1 h under a N₂ atmosphere. The reaction mixture was filtered and concentrated under reduced pressure. The residue was purified by prep-TLC (SiO₂, EtOAc: MeOH=5:1) to give 2-[(2S)-4-[2-[I(2S)-1-[3-hydroxy-2-(hydroxymethyl)propylpyrrolidin-2-yl]methoxy]-7-(8-iodo-1-naphthyl)-6,8-dihydro-5H-pyrido(3,4-d]pyrimidin-4-yl]-1-prop-2-enoyl-piperazin-2-yl]acetonitrile (19 mg, 25.3 μmol, 67% yield) as a white solid: ¹H NMR (400 MHz, DMSO-d₆) δ ppm 1.53-1.77 (4H, m), 1.85-1.94 (1H, m), 2.13-2.22 (1H, m), 2.36 (1H, dd, J=12.19, 4.44 Hz), 2.62-2.70(2H, m), 2.83-2.93(1H, m), 2.96-3.14(3H, m), 3.15-3.28(3H, m), 3.34-3.72(7H, m), 3.79-4.15 (5H, m), 4.22-4.29(1H, m), 4.35-4.51 (1H, m), 4.72-5.06 (1H, m), 5.78 (1H, dd, J=10.6. 1.8 Hz), 6.19 (1H, br d, J=16.6 Hz), 6.75-6.96 (1H, m), 7.17 (1H, t, J=7.8 Hz), 7.38-7.49 (1H, m), 7.57(1H, dt, J=10.1. 7.8 Hz), 7.76 (1H, dd, J=7.6, 4.8 Hz), 7.99 (1H, d, J=8.1 Hz), 8.22 (1H, d, J=6.9 Hz). MS (ES) m/z: 751.2 (M+H)⁺, Rₜ=2.31 min.

It is understood that iodinated compounds used in the above examples can be used for testing and experimental purposes. One skilled in the art understands that the iodo moiety can be replaced with a radioisotope described herein, for example fluorine-18 (¹⁸F), iodine-131 (¹³¹I), iodine-123 (¹²³I), iodine-124 (¹²⁴I), iodine-125 (¹²⁵I), or astatine-211 (²¹¹At).

### Example A20. Synthesis of Radiolabeled Compounds Based on Compounds Ex-1 to Ex-14

Radiolabeled Compound B-8 is synthesized from Compound Ex-1 by procedures depicted in Schemes 1-5.

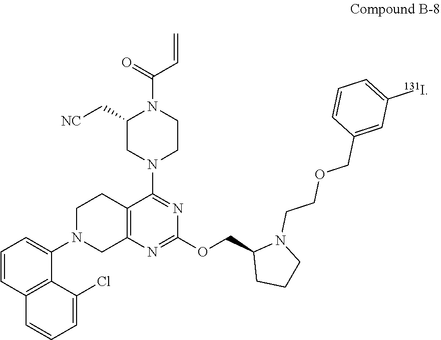

Radiolabeled Compound B-3 is synthesized from Compound Ex-2 by procedures depicted in Schemes 1-5.

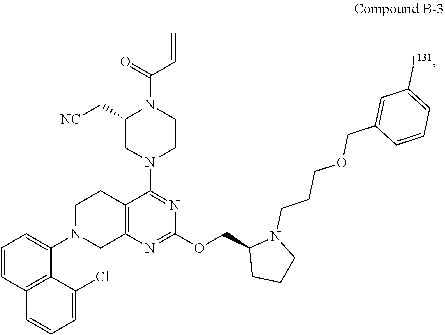

Radiolabeled Compound B-29 is synthesized from Compound Ex-3 by procedures depicted in Schemes 1-5.

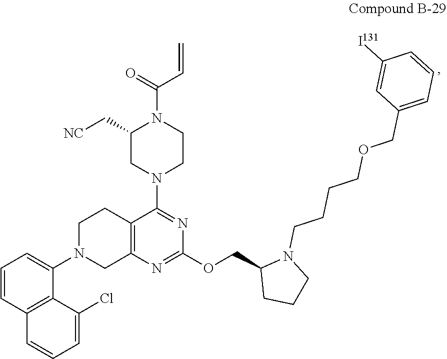

Radiolabeled Compound B-23 is synthesized from Compound Ex-4 by procedures depicted in Schemes 1-5.

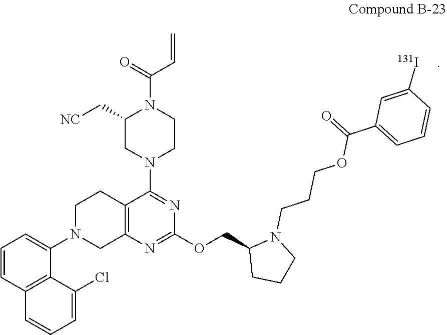

Radiolabeled Compound B-11 is synthesized from Compound Ex-5 by procedures depicted in Schemes 1-5.

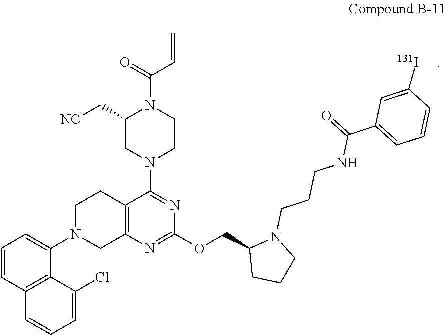

Radiolabeled Compound B-26 is synthesized from Compound Ex-6 by procedures depicted in Schemes 1-5.

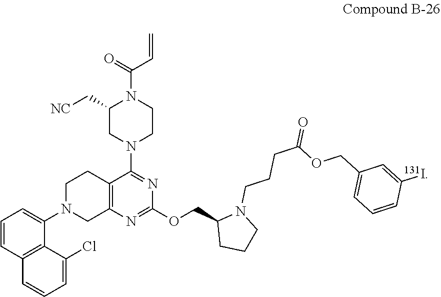

Radiolabeled Compound B-24 is synthesized from Compound Ex-7 by procedures depicted in Schemes 1-5.

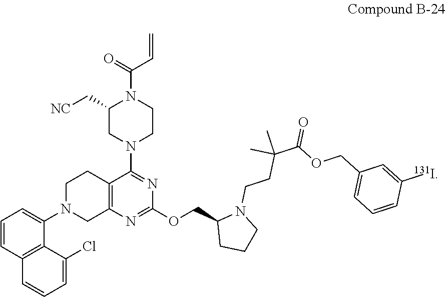

Radiolabeled Compound B-25 is synthesized from Compound Ex-8 by procedures depicted in Schemes 1-5.

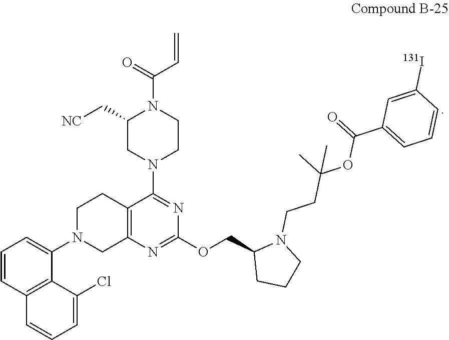 [SMILES: C=CC(=O)N1CCN(c2nc(OC[C@@H]3CCCN3CCC(C)(C)OC(=O)c3cccc([131I])c3)nc3c2CCN(c2cccc4cccc(Cl)c24)C3)C[C@@H]1CC#N]

Radiolabeled Compound A-1 is synthesized from Compound Ex-9 by procedures depicted in Schemes 1-5.

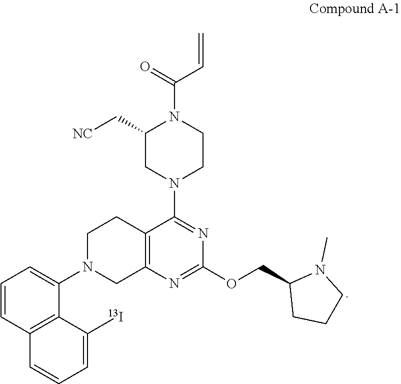

Radiolabeled Compound A-4 is synthesized from Compound Ex-10 by procedures depicted in Schemes 1-5.

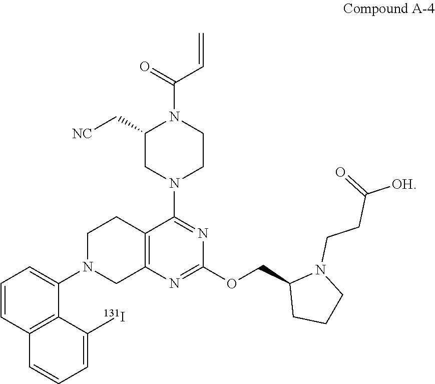 [SMILES: C=CC(=O)N1CCN(c2nc(OC[C@@H]3CCCN3CCC(=O)O)nc3c2CCN(c2cccc4cccc([131I])c24)C3)C[C@@H]1CC#N]

Radiolabeled Compound A-6 is synthesized from Compound Ex-11 by procedures depicted in Schemes 1-5.

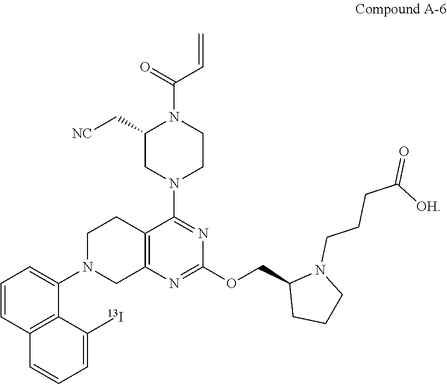

Radiolabeled Compound A-12 is synthesized from Compound Ex-12 by procedures depicted in Schemes 1-5.

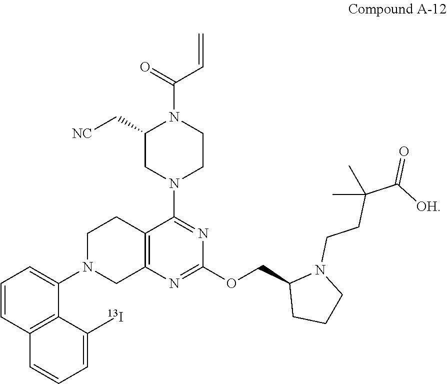

Radiolabeled Compound A-17 is synthesized from Compound Ex-13 by procedures depicted in Compound A-17.

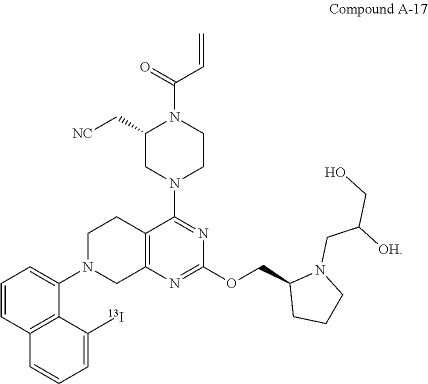

Radiolabeled Compound A-14 is synthesized from Compound Ex-14 by procedures depicted in Compound A-14.

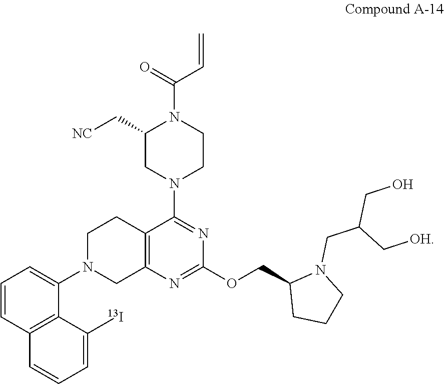

### Example A21. Synthesis of Radiolabeled Compounds of Table 4A, Table 4B, Table 4C, and Table 4D

Radiolabeled compounds of Table 4A, Table 4B, Table 4C, and Table 4D are synthesized by installing the radioisotope on their respective precursor compounds. The precursor compounds are synthesized by methods known in the an, e.g., see Example A1 and Section XVI. Methods of Manufacturing labelled Compounds. The radioisotope is installed according to a method described in Scheme 1, Scheme 2. Scheme 3. Scheme 4, and/or Scheme 5. In some cases, the radioisotope is installed by methods known in the art, see e.g., Berdal et al., “investigation on the reactivity of nucleophilic radiohalogens with arylboronic acids in water: access to an efficient single-step method for the radioiodination and astatination of antibodies” Chemical Science 2021, 12, 1458, and Dubost et al., “Recent Advances in Synthetic Methods for Radioiodination” J. Org. Chem. 2020, 85, 13, 8300-8310.

Alternatively, radiolabeled compounds of Table 4A, Table 4B. Table 4C, and Table 4D are synthesized by installing the radioisotope on their respective precursor compounds, for example, according to schemes 25-32 below.

### A22. Radioiodination Via Aryl Bromide.

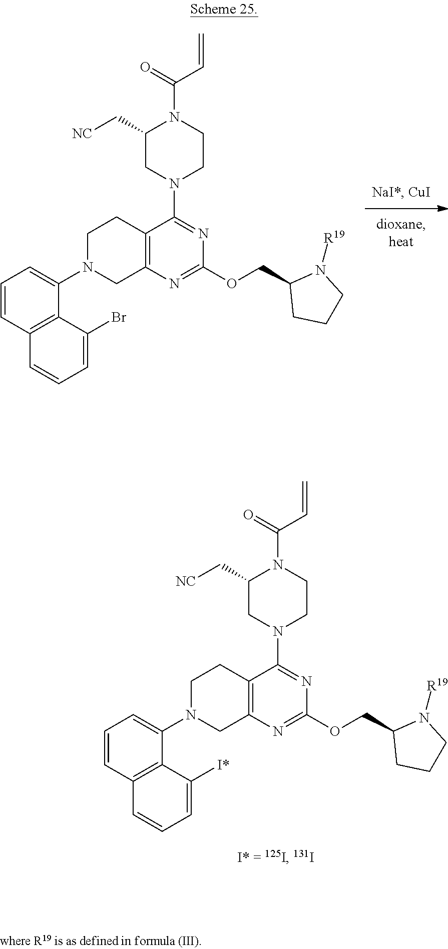

### A23. Radioiodination Via Boronic Acid or Stannane Intermediate.

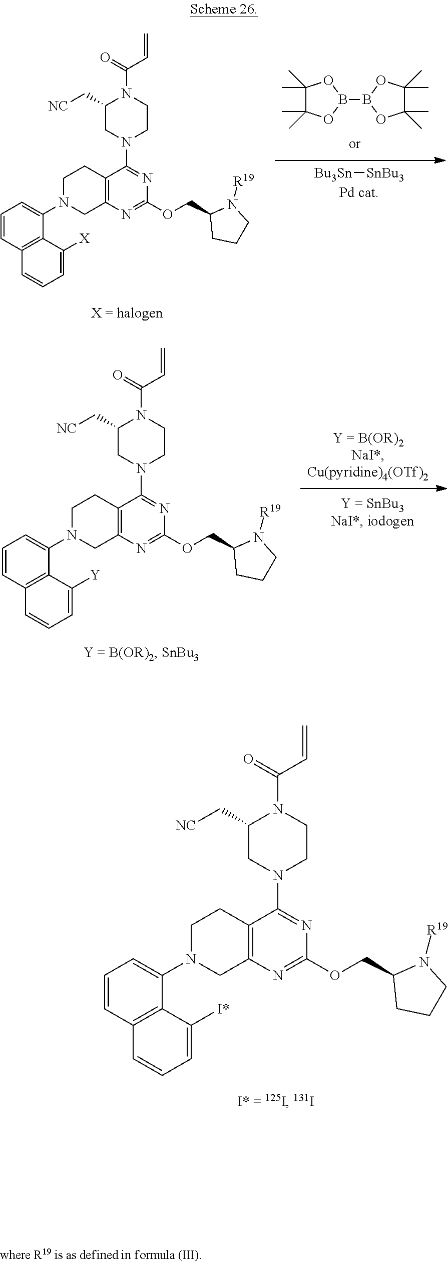

### A24. Radioiodination Via Linker Boronic Acid or Stannane Intermediate.

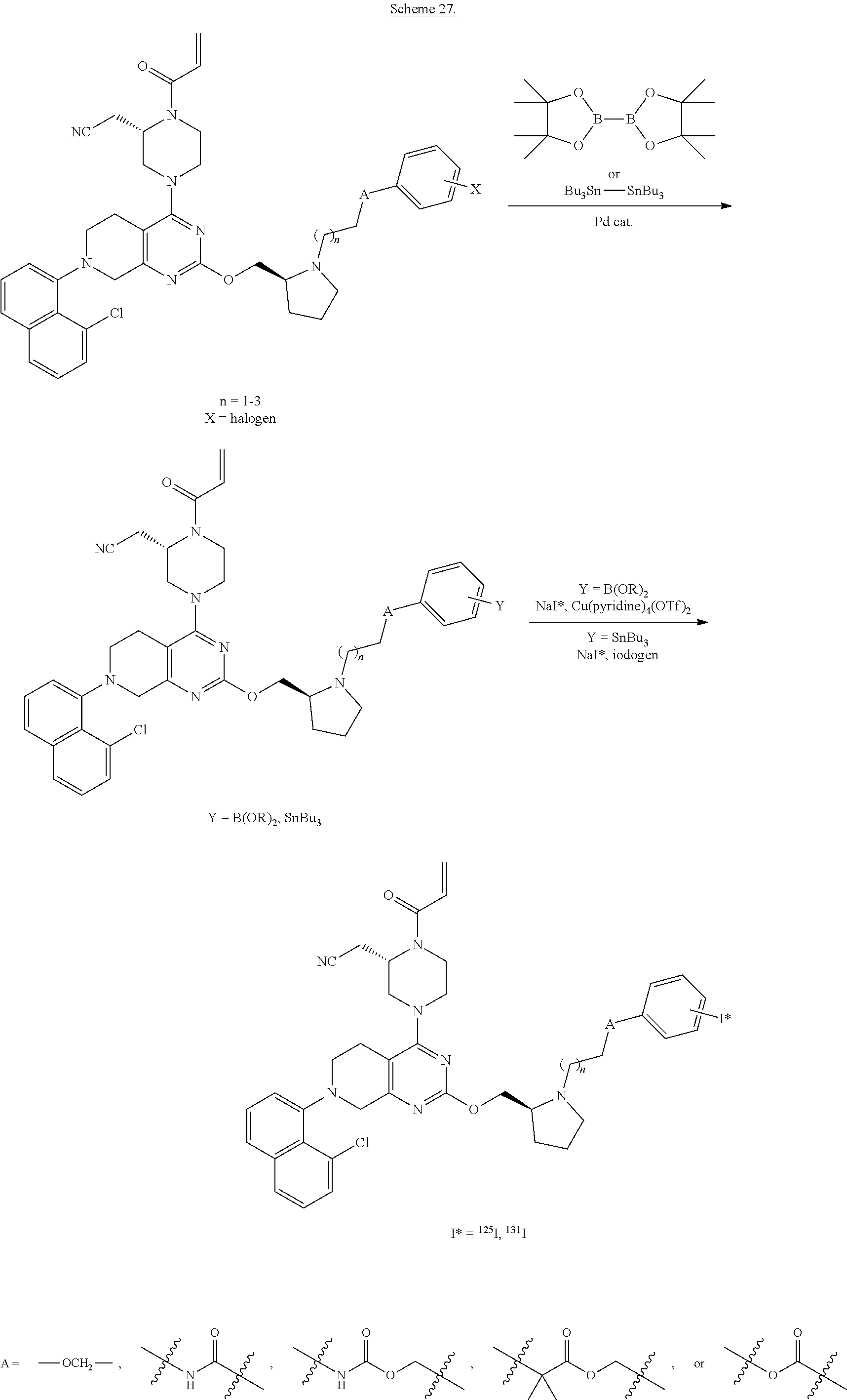

### A25. Radioiodination Via Linker Boronic Acid

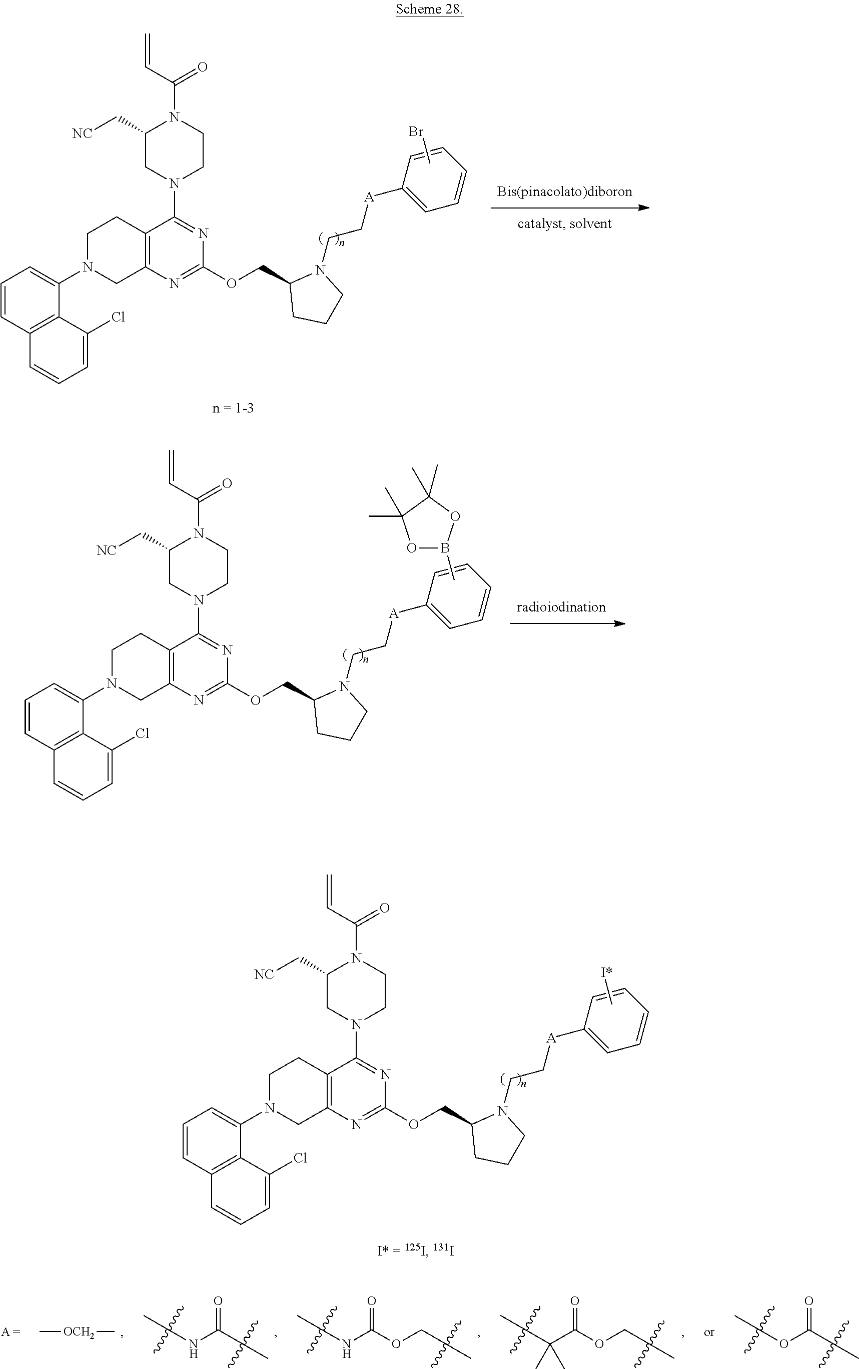

### A26. Radioiodination Via Linker Stannane Intermediate

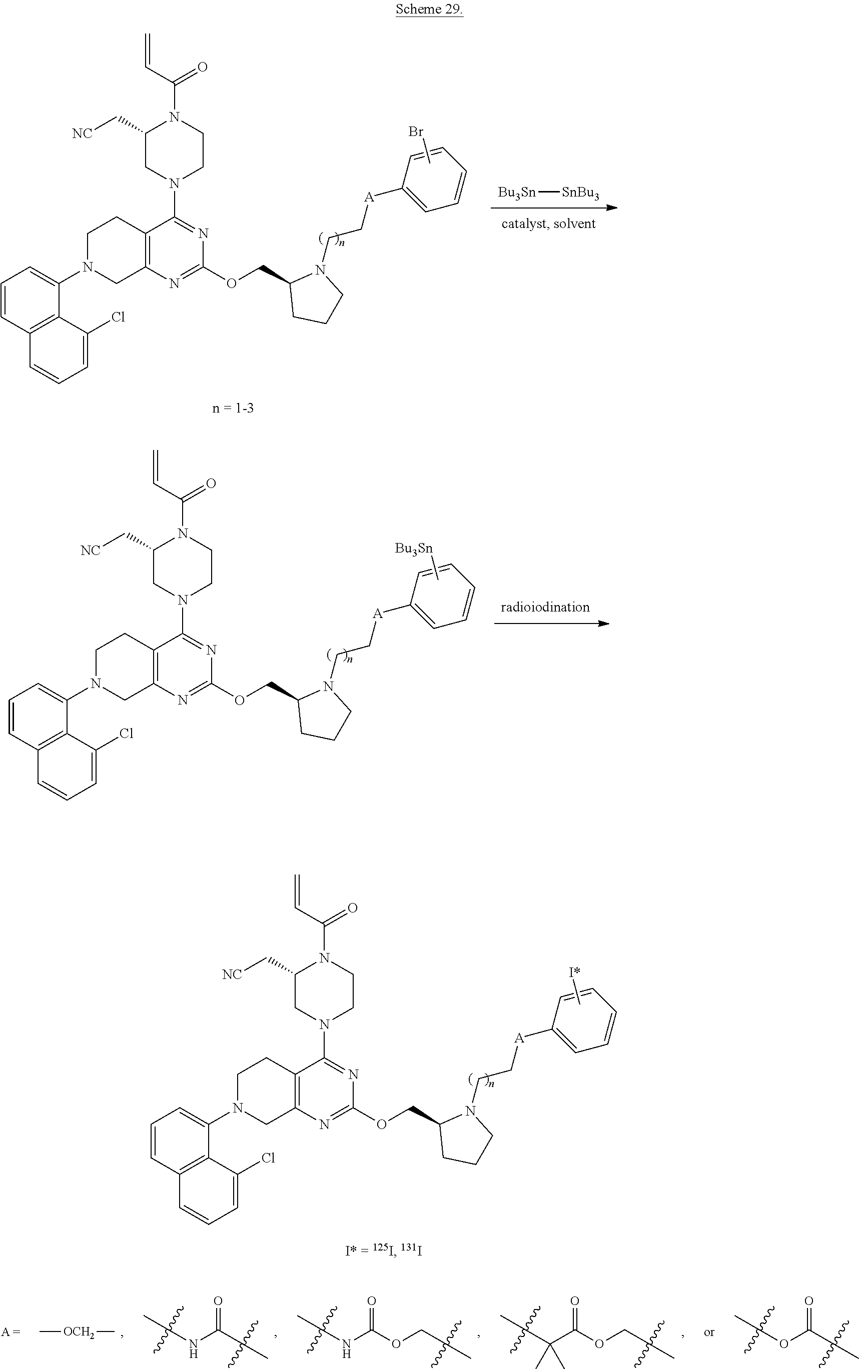

### A27. Radioiodination Via Linker Bromine

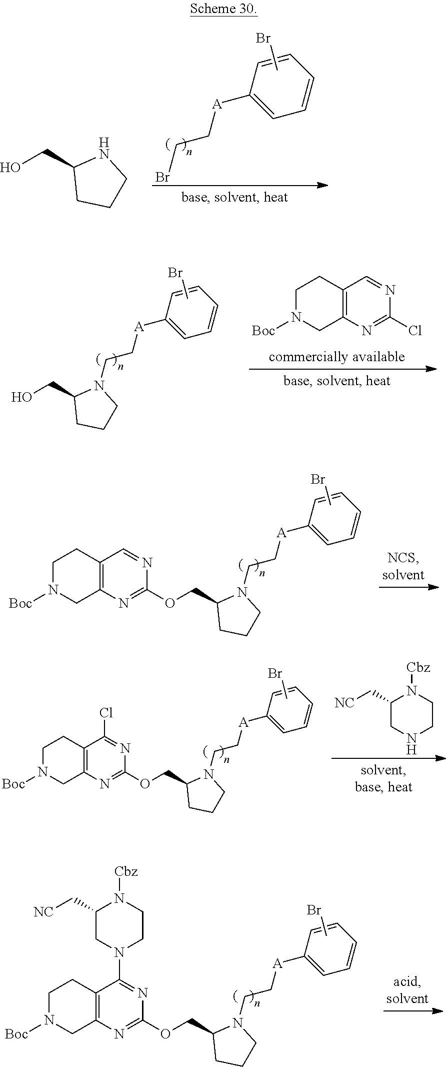

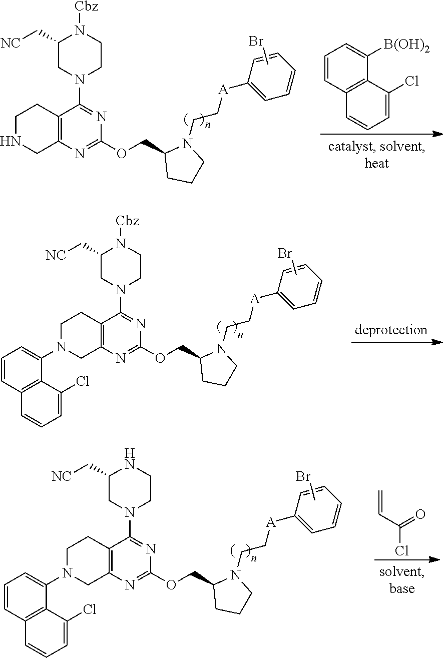

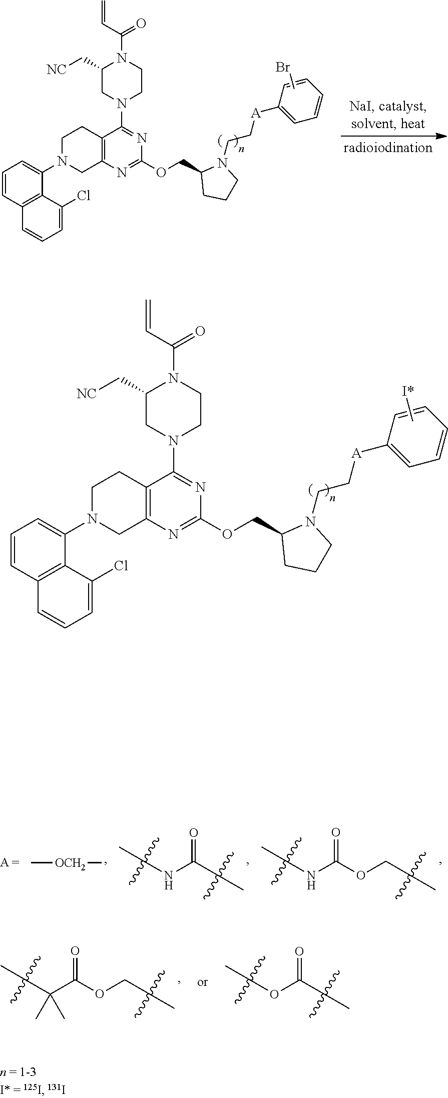

### A28. Radioiodination Via Linker Diazotization Procedure

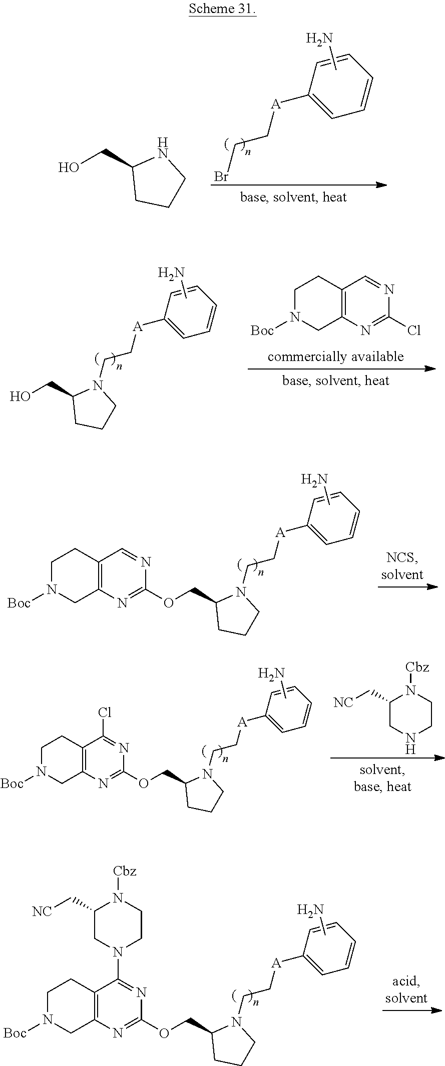

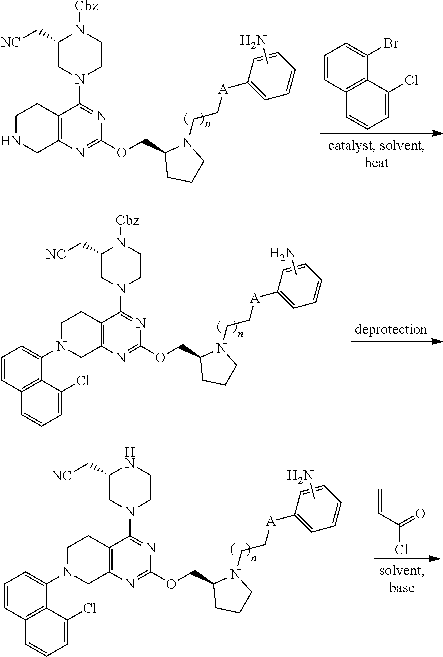

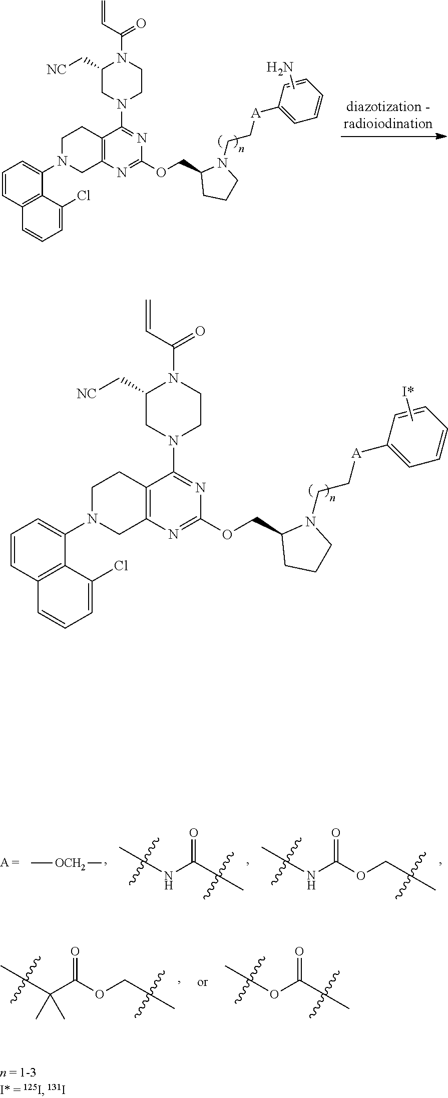

For diazotization procedure for radioiodination see: Sloan, N. L.; Luthra, S. K.; McRobbie, G.; Pimlott. S. L.; Sutherland, A. A One-Pot Radioiodination of Aryl Amines via Stable Diazonium Salts: Preparation of 125I-Imaging Agents. Chem. Commun. 2017. 53 (80), 11008-11011 which is incorporated herein by reference.

### A29. Radioiodination Via Naphthyl Bromine

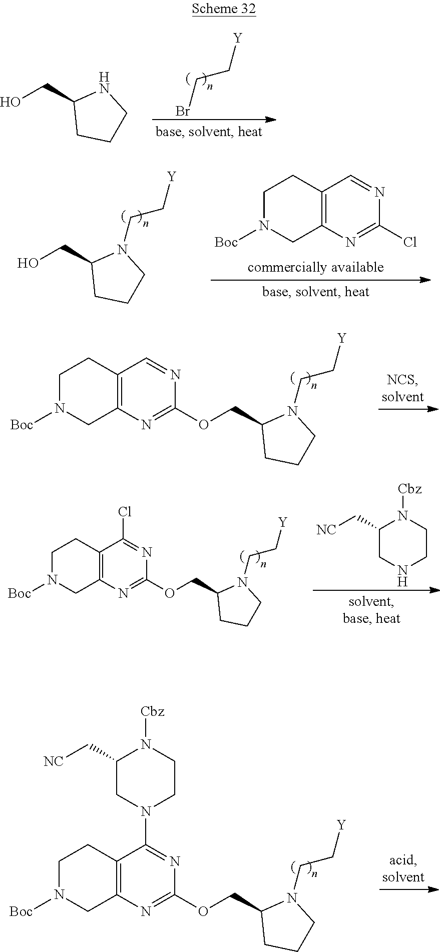

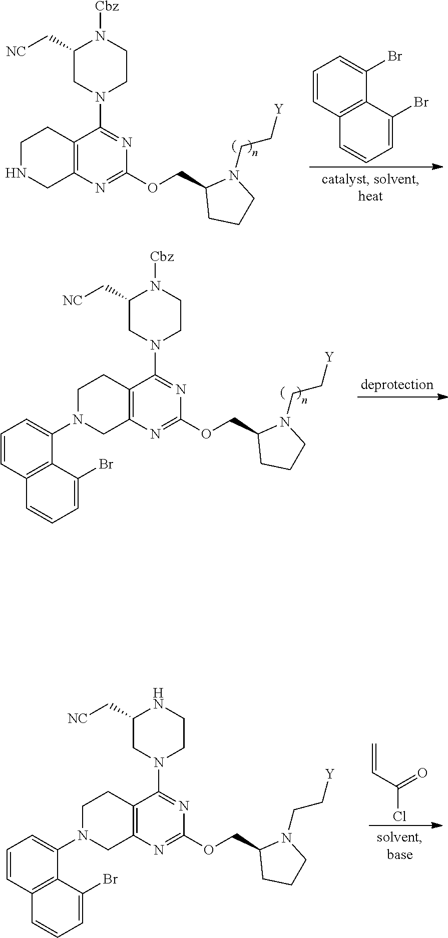

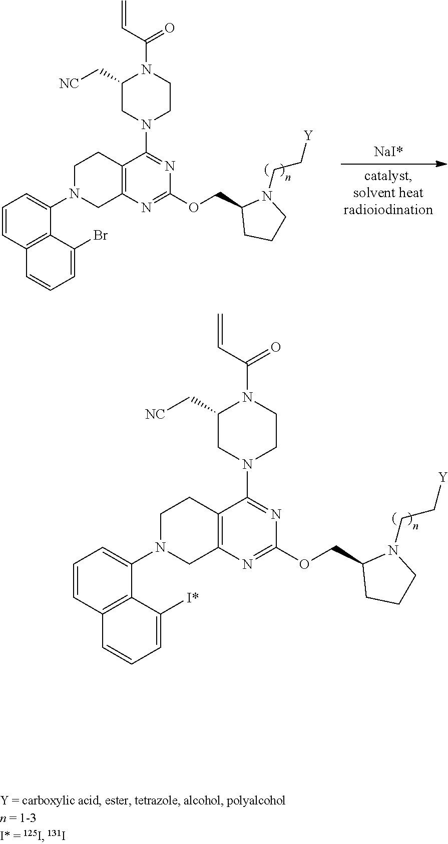

### A30. Preparation of Compound CHL-001 and Lu-CHL-001

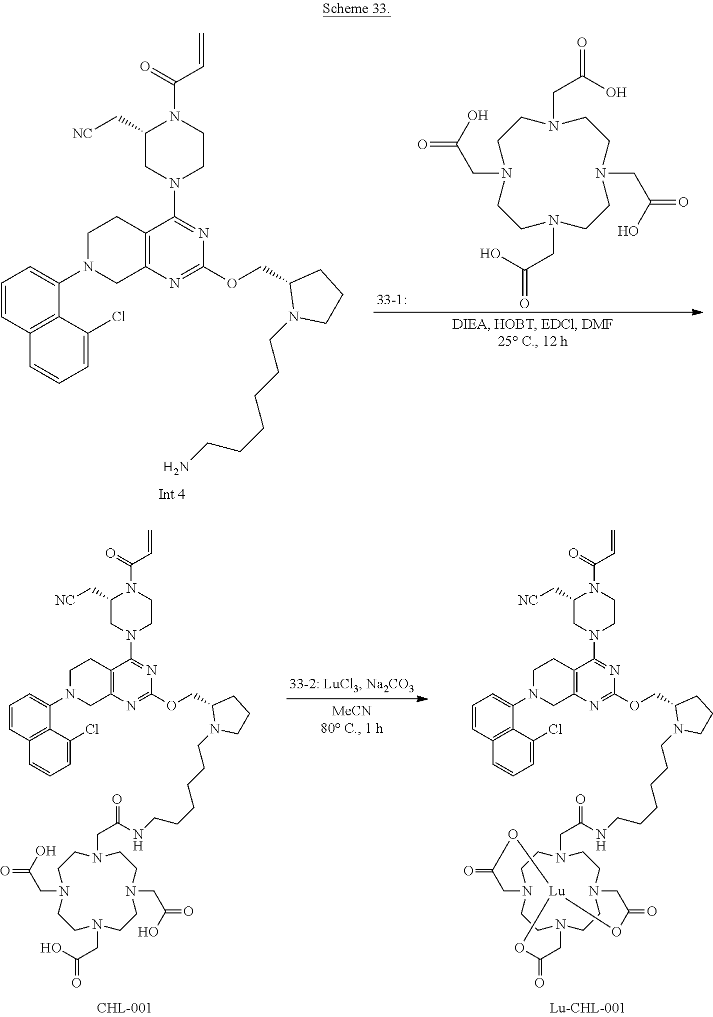

### 33.1 Preparation of Compound CHL-001

To a mixture of 2-[(2S)-4-[2-[[(2S)-1-(6-aminohexyl)pyrrolidin-2-yl]methoxy]-7-(8-chloro-1-naphthyl)-6,8 dihydro-5H-pyrido[3,4-d]pyrimidin-4-yl]-1-prop-2-enoyl-piperazin-2-yl]acetonitrile Int 4 (170 mg, 217 μmol, 1 eq. TFA) and 2-[4,7,10-tris(carboxymethyl)-1,4,7,10-tetrazacyclododec-1-yl]acetic acid (175 mg, 433 μmol, 2.0 eq) in DMF (8 mL) was added DIEA (168 mg, 1.30 mmol, 226 μL, 6.0 eq). HOBt (35.1 mg, 260 μmol, 1.2 eq) and EDCI (91 mg, 476 μmol, 2.2 eq) at 0° C. The reaction mixture was stirred at 25° C. for 12 h under a N₂ atmosphere. The mixture was concentrated under reduced pressure and the residue purified by prep-HPLC to give 2-[4,7-bis(carboxymethyl)-10-[2-[6-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]hexylamino]-2-oxo-ethyl]-1,4,7,10-tetrazacyclododec-1-yl]acetic acid CHL-001 (80 mg, 70 μmol, 33% yield, 93% purity) as a white solid; ¹H NMR (400 MHz. MeOD-d₄): δ (ppm) 1.20-1.78 (8H, m), 1.84-2.37 (6H, m), 2.61-3.21 (18H, m), 3.36-3.98 (20H, m), 4.05-4.46 (4H, m), 4.67 (4H, d, J=5.6 Hz), 5.84 (1H, d, J=10.4 Hz), 6.29 (1H, d, J=16.4 Hz), 6.70-7.00(1H, m), 7.29-7.42 (2H, m), 7.47-7.58 (2H, m), 7.69 (I H, d, J=8.4 Hz), 7.79-7.88(1H, m). MS (ESI) m/z: 529.4 (M/2+H)⁺, Rₜ=2.76 min.

### 33.2 Preparation of Compound Lu-CHL-001

To a mixture of 2-[4,7-bis(carboxymethyl)-10-[2-[6-[(2S)-2-[[7-(8-chloro-1-naphthyl)-4-[(3S)-3-(cyanomethyl)-4-prop-2-enoylpiperazin-1-yl]-6,8-dihydro-5H-pyrido[3,4-d]pyrimidin-2-yl]oxymethyl]pyrrolidin-1-yl]hexylamino]-2-oxo-ethyl]-1,4,7,10-tetrazacyclododec-1-yl]acetic acid CHL-001 (20 mg, 18.9 μmol, 1.11 eq) in MeCN (1 mL) was added aqueous Na₂CO₃ solution (2 M, 9.5 uL, 1.0 eq). Trichlorolutetium (5.3 mg, 18.9 μmol, 1.0 eq) was added to the mixture and stirred at 80° C. for 1 h. The mixture was concentrated under reduced pressure and the residue purified by prep-HPLC to give N-[6-[(2S)-2-[[7-(8-chloro-1-naphthyl)-4-[(3 S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]hexyl]-2-(3,16,19-trioxo-2,17,18-trioxa-5,8,11,14-tetraza-1λ3-lutetatricyclo[9,6,3,25,14]docosan-8-yl)acetamide Lu-CHL-001 (10.4 mg, 8.49 μmol, 44.9% yield, 100% purity) as a yellow solid; ¹H NMR (400 MHz. MeOD-d₄) δ (ppm) 1.33-1. 75 (9H, m), 1.87-2.07 (3H, m), 2.22-2.58 (6H, m), 2.64-2.93 (10H, m), 3.01-3.25 (l OH, m), 3.40-3.81 (12H, m), 4.07-4.36 (4H, m), 4.43-4.66 (4H, m), 4.97-5.16(1H, m), 5.84 (I H, d, J=9.6 Hz), 6.29 (1H, d, J=16.4 Hz), 6.75-6.94 (1H, m), 7.30-7.42 (2H, m), 7.47-7.56 (2H, m), 7.69 (1H, dd, J=8.0, 2.4 Hz), 7.84 (1H, d, J=8.0 Hz), 8.53 (1H, s). MS (ESI) m/z: 615.3 (M/2+H)⁺, Rₜ=2.73 min.

### A31. Preparation of Compound CHL-002 and Lu-CHL-002

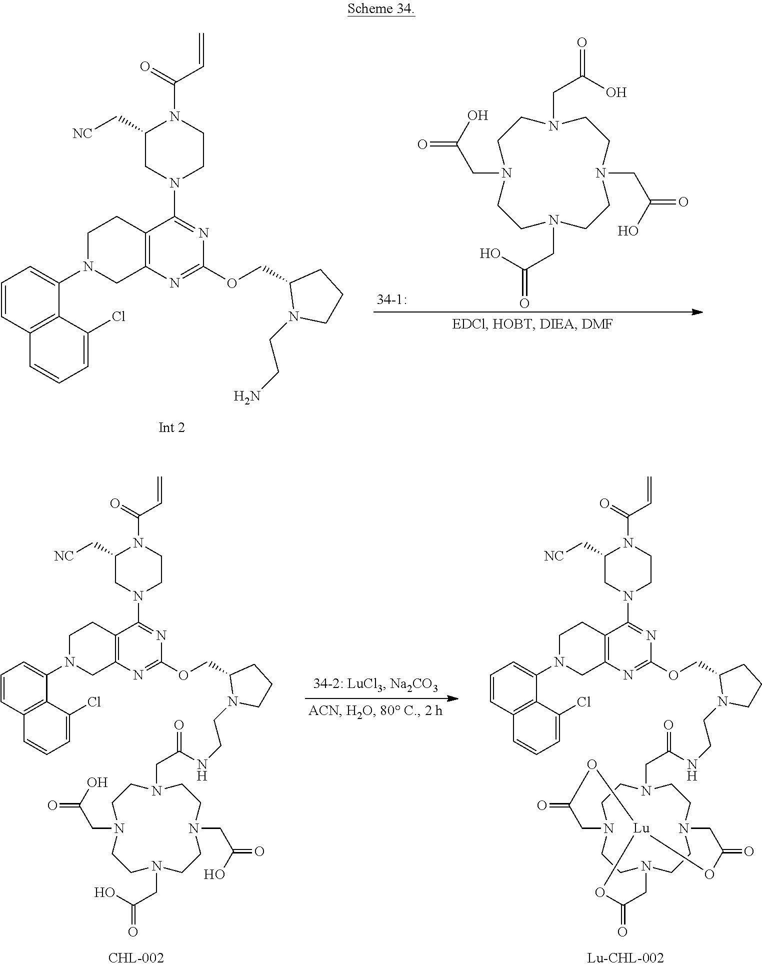

### 34.1 Preparation of Compound CHL-002

A solution of 2-[(2S)-4-[2-[[(2S)-1-(2-aminoethyl)pyrrolidin-2-yl]methoxy]-7-(8-chloro-1-naphthyl)-6,8-dihydro-5H-pyrido[3,4-d]pyrimidin-4-yl]-1-prop-2-enoyl-piperazin-2-yl]acetonitrile (0.15 g, 206 μmol, 1 eq. TFA) in DMF (2 mL) was cooled to 0° C. DIEA (80 mg, 617 μmol, 107 uL, 3 eq), 2-[4,7,10-tris(carboxymethyl)-1,4,7,10-tetrazacyclododec-1-yl]acetic acid (100 mg, 247 μmol, 1.2 eq). HOBt (42 mg, 309 μmol, 1.5 eq) and EDCI (79 mg, 411 μmol, 2 eq) were added and the reaction stirred at 25° C. for 1 h. The reaction was filtered and the filtrate purified by prep-HPLC to give 2-[4,7-bis(carboxymethyl)-10-[2-[2-[(2S)-2-[[7-(8 chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]ethylamino]-2-oxomethyl]-1,4,7,10-tetrazacyclododec-1-yl]acetic acid (50 mg, 49 μmol, 24% yield) as a yellow solid; ¹H NMR (400 MHz, METHANOL-di) δ ppm 2.01-2.42 (4H, m), 2.45-3.32 (18H, m), 3.36-3.87 (19H, m), 3.88-4.00(1H, m), 4.03-4.18(3H, m), 4.19-4.45 (3H, m), 4.49-4.77 (2H, m), 4.97-5.22 (2H, m), 5.64-6.02 (1H, m). 6.22-6.49 (1H, m), 6.69-7.14 (1H, m), 7.31-7.46 (2H, m), 7.48-7.61 (2H, m), 7.68-7.75 (1H, m), 7.80-7.90 (1H, m), 8.42 (1H, s). MS (ESI) m/z: 515.7 (M/2+H)⁺, Rₜ=0.51 min.

### 34.2 Preparation of Compound Lu-CHL-002

A solution of 2-[4,7-bis(carboxymethyl)-10-[2-[2-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]ethylamino]-2-oxo-ethyl]-1,4,7,10-tetrazacyclododec-1-yl]acetic acid (40 mg, 40 μmol, 0.6 eq) in H₂O (1 mL) and CH₃CN (2 mL) was adjusted to pH=6 with Na₂CO₃ (10.6 mg, 99.8 μmol, 1.5 cry). LuCl₃ (18.7 mg, 66.6 umol, 1 eq) was added and the reaction stirred at 80° C. for 2 h. The filtrate was purified by prep-HPLC to give N-[2-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]ethyl]-2-(3,16,19-trioxo-2,17,18-trioxa-5.8,11,14-tetraza-1λ3-lutetatricyclo[9.6.3,25,14]docosan-8-yl)acetamide (9.0 mg, 7.5 μmol, 11% yield) as a yellow solid; ¹H NMR (400 MHz, CD₃OD) δ ppm 1.69-2.90 (21H, m), 3.12-3.35 (8H, m), 3.42-3.81 (16H, m), 4.05-4.56 (6H, m), 5.01-5.20 (1H, m), 5.79-6.06 (1H, m), 6.24-6.43 (1H, m), 6.67-7.08 (1H, m), 7.31-7.45 (2H, m), 7.47-7.61 (2H, m), 7.68-7.76 (1H, m), 7.80-7.90 (1H, m), 8.41 (2H, s). MS (ESI) m/z: 587.4 (M/2+H)⁺, Rₜ=2.12 mm.

### A32. Synthesis of CHL-003 and Lu-CHL-003

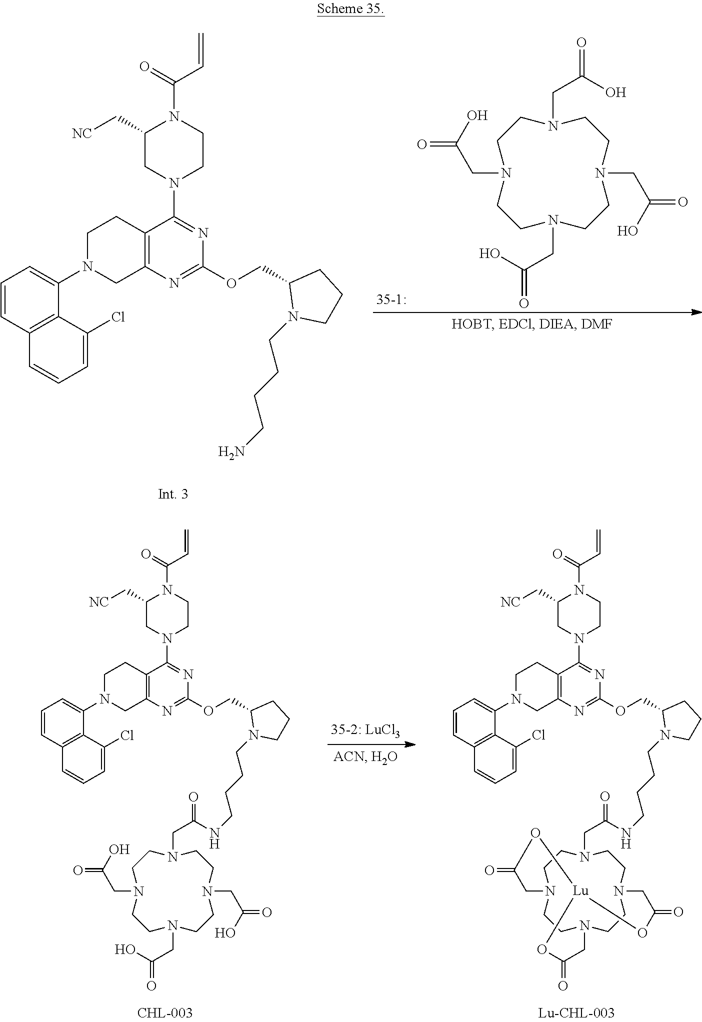

### 35.1 Preparation of Compound CHL-1103

To a solution of 2-[(2S)-4-[2-[[(2S)-1-(4-aminobutyl)pyrrolidin-2-yl]methoxy]-7-(8-chloro-1-naphthyl)-6,8-dihydro-5H-pyrido[3,4-d]pyrimidin-4-yl]-1-prop-2-enoyl-piperazin-2-yl]acetonitrile (0.20 g, 264 μmol) in DMF (4 mL) was added DIEA (171 mg, 1.32 mmol), 2-[4,7,10-tris(carboxymethyl)-1,4,7,10-tetrazacyclododec-1-yl]acetic acid (214 mg, 528 μmol). HOBt (71 mg, 528 μmol) and EDCI (101 mg, 528 umol). The mixture was stirred at 25° C. for 1 h. The reaction mixture was purified by prep-HPLC to give 2-[4,7-bis(carboxymethyl)-10-[2-[4-[(2S)-2-[[7-(8-chloro-1-napthyl)-4-[(3S)-3-(cyanomethyl)-4-prop-2-enoylpiperazin-1-yl]-6,8-dihydro-5H-pyrido[3,4-d]pyrimidin-2-yl]oxymethyl]pyrrolidin-1-yl]butylamino]-2-oxo-ethyl]-1,4,7,10-tetrazacyclododec-1-yl]acetic acid (60 mg, 22% yield) as a yellow gum: ¹H NMR (400 MHz, CD₃OD): δ (ppm) 1.49-1.90 (4H, m), 1.94-2.34 (6H, m), 2.72-3.22 (18H, m), 3.41-3.87 (18H, m), 4.07-4.75 (9H, m), 5.02-5.23 (1H, m), 5.84 (1H, d, J=10.8 Hz), 6.18-6.44 (1H, m), 6.72-7.01 (1H, m), 7.30-7.42(2H, m), 7.45-7.58 (2H, m), 7.69(1H, d, J=8.0 Hz), 7.83 (1H, d, J=8.0 Hz). MS (ESI) m/z: 515.7 (M/2+H. Rₜ=0.51 min.

### 35.2 Preparation of Lu-CHL-003

To a solution of 2-[4,7-bis(carboxymethyl)-10-[2-[4-[(2S)-2-[[7-(8-chloro-1-naphthyl)-4-[(3S)-3-(cyanomethyl)-4-prop-2-enoylpiperazin-1-yl]-6,8-dihydro-5H-pyrido[3,4-d]pyrimidin-2-yl]oxymethyl]pyrrolidin-1-yl]butylamino]-2-oxo-ethyl]-1,4,7,10-tetrazacyclododec-1-yl]acetic acid (35 mg, 34 μmol) in CH₃CN (1 mL) and H₂O (1 mL) was added LuCl₃ (10 mg, 35 μmol). The mixture was stirred at 80° C. for 2 h. The residue was purified by prep-HPLC to give N-[4-[(2S)-2-[[7-(8-chloro-1-naphthyl)-4-[(3S)-3-(cyanomethyl)-4-prop-2-enoyl-piperazin-1-yl]-6,8-dihydro-5Hpyrido[3,4-d]pyrimidin-2-yl]oxymethyl]pyrrolidin-1-yl]butyl]-2-(3,16,19-trioxo-2,17,18-trioxa-5,8,11,14-tetrazaλ3-lutetatricyclo[9.6.3,25,14]docosan-8-yl)acetamide (2 mg, 5% yield) as a white solid. ¹H NMR (400 MHz, CD₃OD): δ (ppm) 1.62-2.30 (10H, m), 2.48-3.15 (20H, m), 3.43-3.70 (10H, m), 3.77-4.22 (8H, m), 4.29-4.67 (6H, m), 5.81-5.9 E (1H, m), 6.25-6.40(111, m), 6.75-6.95 (1H, m), 7.36-7.44 (2H, m), 7.48-7.60 (2H, m), 7.73 (1H, d, J=8.4 Hz), 7.87 (1H, d, J=8.4 Hz). MS (ESI) m/z: 1202.3 (M+H)⁺, Rₜ=0.82 min.

### A33. Synthesis of CHL-004

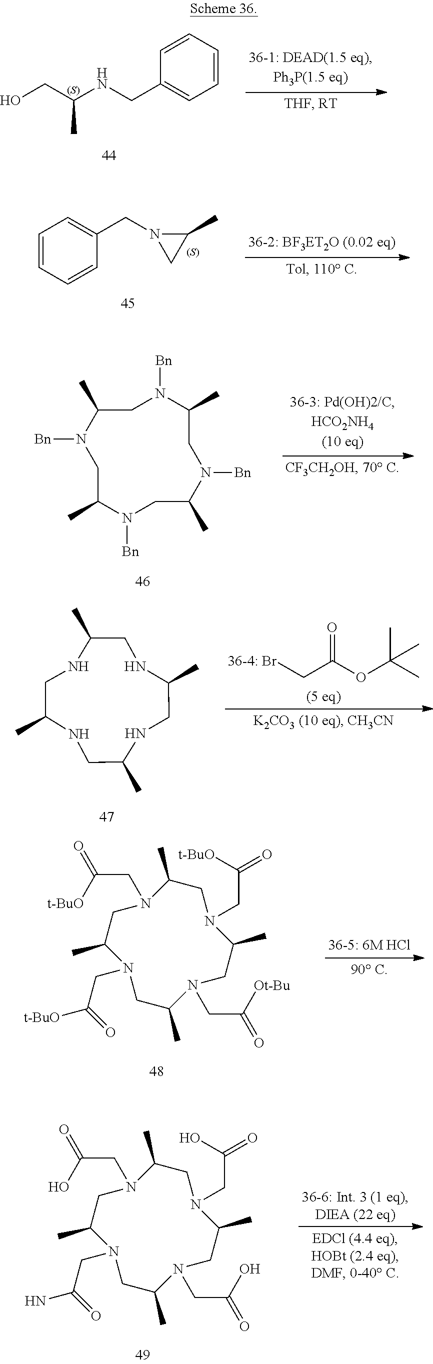

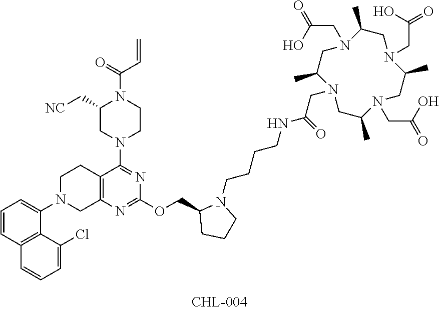 [SMILES: C=CC(=O)N1CCN(c2nc(OC[C@@H]3CCCN3CCCCNC(=O)CN3C[C@H](C)N(CC(=O)O)C[C@H](C)N(CC(=O)O)C[C@H](C)N(CC(=O)O)C[C@@H]3C)nc3c2CCN(c2cccc4cccc(Cl)c24)C3)C[C@@H]1CC#N]

### 36.1 Preparation of Compound 45

A mixture of (2S)-2-(benzylamino)propan-1-ol 44 (20 g, 121 mmol, 1.0 eq) and PPh₃ (47.6 g, 182 mmol, 1.5 eq) in THE (200 mL) was added DEAD (31.6 g, 182 mmol, 33 mL, 1.5 eq) at 0° C., the mixture purged with N₂ 3 times and stirred at 15° C. for 16 h under a N₂ atmosphere. The pH of the aqueous phase was adjusted to pH-5 by adding citric acid solution, extracted with EtOAc 300 mL (50 mL×6), the pH of the aqueous phase adjusted to pH-8 and the solution extracted with EtOAc (50 mL×6). The combined organics were dried over Na₂SO₄, filtered and concentrated under reduced pressure to give (2S)-1-benzyl-2-methyl-aziridine (11.4 g, 77.4 mmol, 64% yield) as a yellow oil; ¹H NMR (400 MHz, CHLOROFORM-d) δ ppm 1.22 (3H, d, J=5.50 Hz), 1.40(1H, d, J=6.25 Hz), 1.49-1.56 (1H, m), 1.59 (1H, d, J=3.6 Hz), 3.28-3.56(2H, m), 7.25-7.29(1H, m), 7.30-7.40(4H, m). MS(ESI)ₙ/z: 148.1 (M+H)⁺, Rₜ=1.41 min.

### 36.2 Preparation of Compound 46

A mixture of (2S)-1-benzyl-2-methyl-aziridine 45(11.0 g, 74.7 mmol, 1 eq) and BF₃·Et₂O (215 mg, 1.52 mmol, 187 μL, 0.02 eq) in toluene (48 mL) was degassed and purged with N₂ 3 times. The mixture was stirred at 110° C. for 5 h. The reaction mixture was concentrated under reduced pressure. The residue was diluted with DCM (50 mL) and washed with water (101 mL). The combined organic layers were dried over Na₂SO₄, filtered and concentrated under reduced pressure. The residue was purified by prep-HPLC to give (2S,5S, 8S,11S)-1.4,7,10-tetrabenzyl-2,5,8,11-tetramethyl-1,4,7,10-tetrazacyclododecane (3.92 g, 6.66 mmol, 8.9% yield) as a white solid; ¹H NMR (400 MHz, CHLOROFORM-ci) δ ppm 0.99 (12H, d, J=6.6 Hz), 2.27 (4H, dd, J=14.3, 2.5 Hz), 3.08 (4H, dd, J=14.1. 11.3 Hz), 3.30 (4H, td, J=6.9, 3.2 Hz), 3.45-3.71 (8H, m), 7.10 (8H, dd, J=6.2, 2.7 Hz), 7.26-7.34 (12H, m). MS (ES) m/z: 589.5 (M+H)⁴, R, -0.69 min.

### 36.3 Preparation of Compound 47

A mixture of (2S,5S, 8S,11S)-1,4,7,10-tetrabenzyl-2,5,8,11-tetramethyl-1,4,7,10-tetrazacyclododecane 46 (1.0 g, 1.70 mmol, 1 eq), ammonium formate (1.07 g, 17.0 mmol, 10 eq) and Pd(OH)₂/C (1.67 g, 2.38 mmol, 20°l° purity, 1.40 eq) in trifluoroethanol (3 mL) was stored at 70° C. for 20 h. The reaction mixture was filtered and concentrated under reduced pressure to give crude (2S,5S, 8S,11S)-2,5,8,11-tetramethyl-1,4,7,10-tetrazacyclododecane (1.0 g) as a white solid; ¹H NMR (400 MHz, CHLOROFORM-d) δ ppm 1.23 (12H, br d, J=3.22 Hz), 2.91 (12H, br s), 8.43-8.81 (4H, m). MS (ESI) m/z: 229.2 (M+H)⁺, Rₜ=0.24 min.

### 36.4 Preparation of Compound 48

A mixture of (2S,5S, 8S,11S)-2,5,8,11-tetramethyl-1,4,7,10-tetrazacyclododecane (1.0 g, 4.38 mmol, 1 eq), tert-butyl 2-bromoacetate 47 (4.27 g, 21.9 mmol, 3.24 mL, 5 eq) and K₂CO₃ (6.05 g, 43.8 mmol, 10 eq) in CH₃CN (2 mL) was degassed and purged with N₂ 3 times. The reaction was stirred at 50° C. for 16 h under a N₂ atmosphere. The reaction was cooled to room temperature, filtered and concentrated under reduced pressure. The residue was dissolved into 2% HCl (60 mL) and extracted with EtOAc (30×2 mL). The pH of the aqueous phase was adjusted to pH-8, then extracted with DCM (30×2 mL). The combined organic phases were dried over Na₂SO₄, filtered, the filtrate concentrated under reduced pressure and the residue purified by prep-HPLC to give tert-butyl 2-[(2S,5S,8S,11S)-4,7,10-tris(2-tert-butoxy-2-oxo-ethyl)-2,5,8,11-tetramethyl-1,4,7,10-tetrazacyclododec-1-yl]acetate (0.40 g, 584 μmol, 13% yield) as a white solid; ¹H NMR (400 MHz, CHLOROFORM-d) δ ppm 0.83-1.26 (12H, m), 1.36-1.59 (36H, m), 2.02-4.21 (20H, m). MS (ESI) m/z: 684.5 (M+H)⁺, Rₜ=0.90 min.

### 36.5 Preparation of Compound 49

A mixture of tert-butyl 2-[(2S,5S,8S,11S)-4,7,10-tris(2-tert-butoxy-2-oxo-ethyl)-2,5,8,11-tetramethyl-1,4,7,10-tetrazacyclododec-1-yl]acetate 48 (0.30 g, 438 μmol, 1 eq) in HCl (6 M, 2.00 mL) was degassed and purged with N₂ 3 times, and stirred at 90° C. for 2 h under a N₂ atmosphere. The reaction mixture was concentrated under reduced pressure and the residue purified by prep-HPLC to give 2-[(2S,5S, 8S, 11S)-4,7,10-tris(carboxymethyl)-2,5,8,11-tetramethyl-1,4,7,10-tetrazacyclododec-1-yl]acetic acid (0.10 g, 217 μmol, 50% yield) as a white solid: ¹H NMR (400 MHz, CHLOROFORM-ti) δ ppm 0.92-1.41 (11H, m), 2.75-4.17 (21H, m). MS (ESI) m/z: 460.2 (M+H, Rₜ=0.25 min.

### 36.6 Preparation of Compound CHL-004

To a solution of 2-[(2S,5S,8S,11S)-4,7,10-tris(carboxymethyl)-2,5,8,11-tetramethyl-1,4,7,10-tetrazacyclododec-1-yl]acetic acid 49 (70 mg, 152 μmol, 2 eq) in DMF (2 mL) and 2-[(2S)-4-[2-[[(2S)-1-(4-aminobutyl)pyrrolidin-2-yl]methoxy]-7-(8-chloro-1-naphthyl)-6,8-dihydro-5H-pyrido[3,4-d]pyrimidin-4-yl]-1-prop-2-enoyl-piperazin-2-yl]acetonitrile (49 mg, 76.0 μmol, 1 eq) was added DIEA (196 mg, 1.52 mmol, 265 μL, 20 eq), HOBt (25 mg, 182 μmol, 2.4 eq) and EDCI (64 mg, 334 μmol, 4.4 eq) at 0° C. The mixture was stirred at 40° C. for 16 h. The reaction mixture was concentrated under reduced pressure and the residue purified by prep-HPLC to yield 2-[(2S,5S,8S, 11S)-7,10-bis(carboxymethyl)-4-[2-[4-[(2S)-2-[[7-(8-chloro-1-naphthyl)-4-[(3 S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]butylamino]-2-oxo-ethyl]-2,5,8,11-tetramethyl-1.4,7,10-tetrazacyclododec-1-yl]acetic acid (40 mg, 36.8 μmol, 48% yield) as a white solid: ¹H NMR (400 MHz, CHLOROFORM-ti) δ ppm 0.99-1.39 (14H, m), 1.55-1.71 (2H, m), 1.80-2.22 (6H, m), 2.31-2.34 (1H, m), 2.33-2.42(1H, m), 2.64-3.28 (17H, m), 3.46-3.52(2H, m) 3.57-3.68 (4H, m) 3.72-3.83(5H, m) 3.86-4.05 (5H, m) 4.15-4.45 (4H, m) 4.55-4.80 (4H, m), 5.86 (1H, br d, J=10.8 Hz), 6.31 (1H, br d, J=17.1 Hz), 6.73-6.91 (1H, m), 7.33-7.43 (2H, m), 7.49-7.58 (2H, m), 7.73 (1H, dd, J=8.3, 2.1 Hz), 7.86 (1H, d, J=8.3 Hz). MS (ESI) m/z: 1084.5 (M+H)⁺, Rₜ=2.06 min.

### A34. Synthesis of CHL-005 and Lu-CHL-005

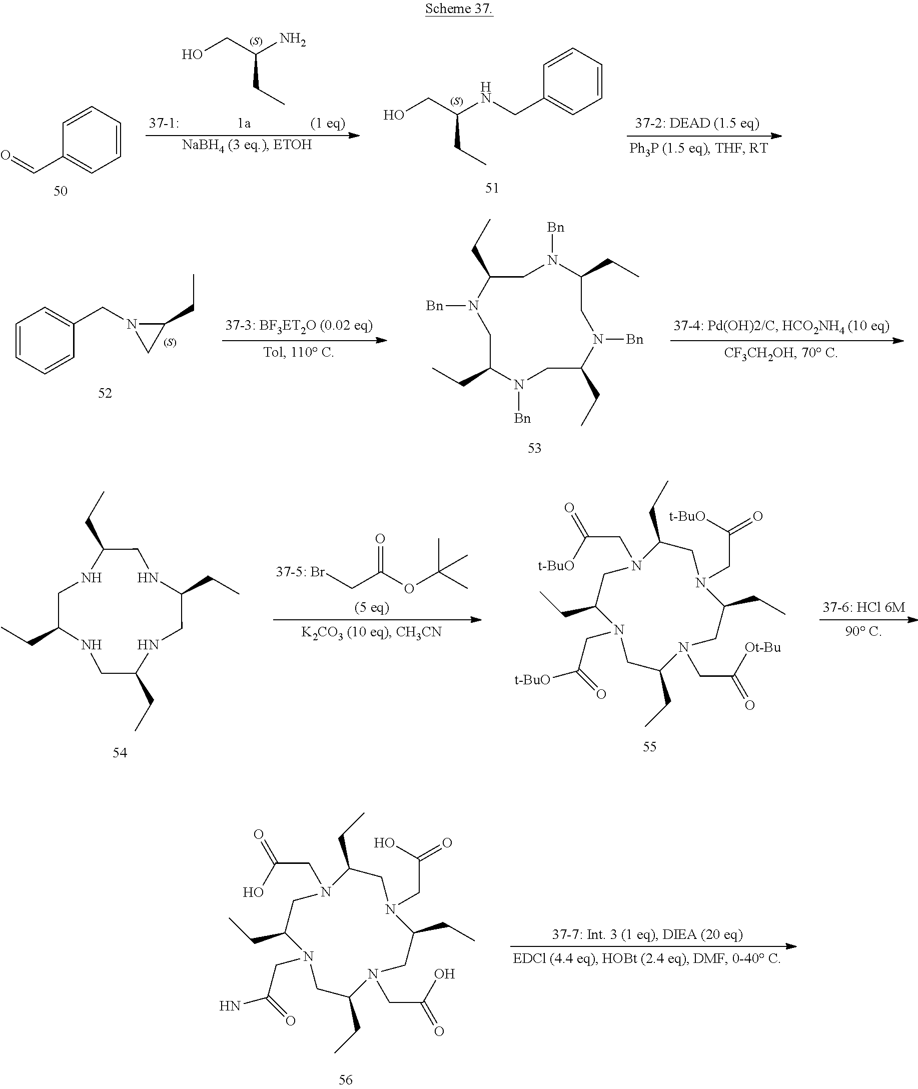

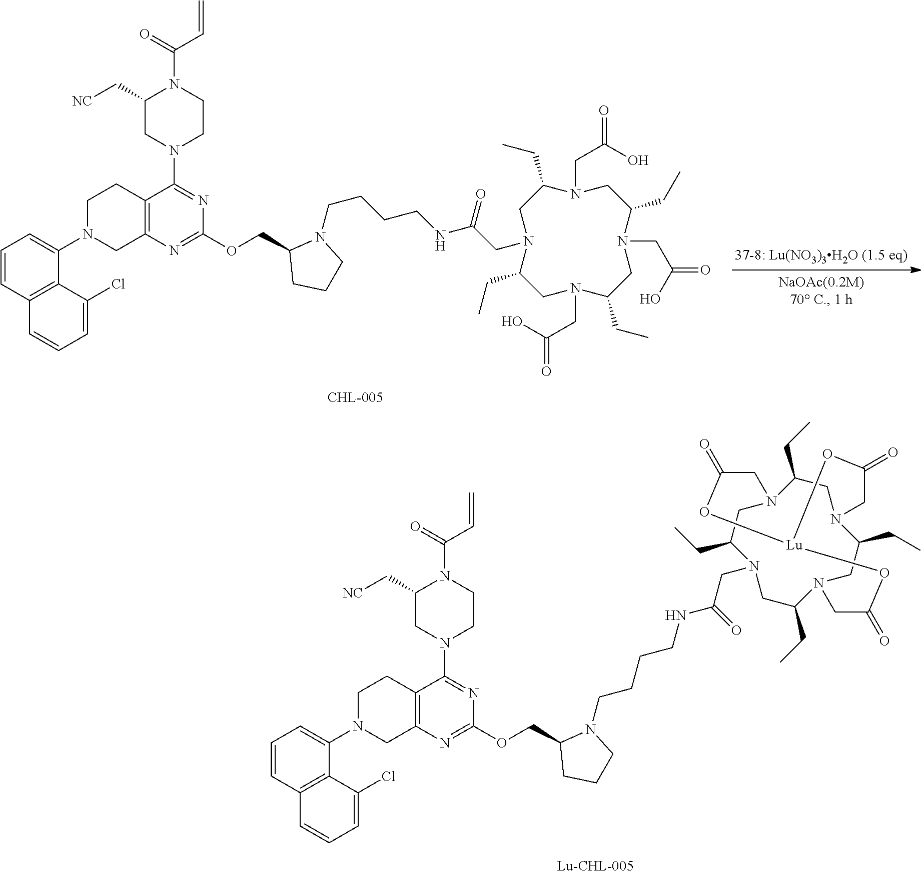

### 37.1 Preparation of Compound 51

To a solution of benzaldehyde (20 g, 188 mmol, 19.1 mL, 1 eq) and (2S)-2-aminobutan-1-ol (16.8 g, 188 mmol, 17.9 mL, 1 eq) in EtOH (200 mL) was added NaBH₄ (21.4 g, 565 mmol, 3 eq) at 0° C., and the mixture stirred at 20° C. for 3 h under a N₂ atmosphere. The reaction mixture was quenched with H₂O (800 mL) and extracted with DCM (200 mL×3). The combined organic layers were washed with brine (100 mL), dried over Na₂SO₄, filtered, concentrated under reduced pressure and purified by column chromatography (SiO₂, petroleum ether:EtOAc=20:1 to 5:1) to give (2S)-2-(benzylamino)butan-1-01(29.6 g, 165 mmol, 88% yield) as a white solid; ¹H NMR (400 MHz, CHLOROFORM-d) δ ppm 0.87 (3H, t, J=7.46 Hz), 1.33-1.59 (2H, m), 2.54-2.64 (1H, m), 3.28 (1H, dd, J=10.64. 6.36 Hz), 3.60 (1H, dd, J=10.64, 3.91 Hz), 3.68-3.82 (2H, m), 7.17-7.32 (5H, m). MS (ESI) m/z: 180.1 (M+H)⁺. Rₜ=0.99 min.

### 37.2 Preparation of Compound 52

To a solution of (2S)-2-(benzylamino)butan-1-ol 51 (15 g, 83.7 mmol, 1.0 eq) and PPh₃ (32.9 g, 126 mmol, 1.5 eq) in THE (150 mL) was added DEAD (21.9 g, 126 mmol, 22.8 mL, 1.5 eq) at 0° C. The reaction was purged with N; 3 times, and the mixture stirred at 20° C. for 16 h under a N₂ atmosphere. The pH of the aqueous phase was adjusted to pH-S by adding citric acid solution, extracted with EtOAc (50 mL×6), the pH of the aqueous phase was adjusted to pH˜8 and extracted with EtOAc (50 mL×6). The combined organic phases were dried over Na₂SO₄, filtered and concentrated under reduced pressure to give (2S)-1-benzyl-2-ethyl-aziridine (11.6 g, 71.6 mmol, 86% yield) as a yellow oil; ¹H NMR (400 MHz, CHLOROFORM-d δ ppm 0.85-0.95 (3H, m), 1.36-1.50 (4H, m), 1.63 (1H, d, J=2.9 Hz), 3.33 (1H, d, J=13.4 Hz), 3.52 (1H, d, J=13.3 Hz), 7.16-7.58 (5H, m). MS (ESI) m/z: 162.1 (M+H)⁺, Rₜ=1.65 min.

### 37.3 Preparation of Compound 53

A mixture of (2S)-1-benzyl-2-ethyl-aziridine 52(11.0 g, 68.2 mmol, 1 eq) and BF₃·Et₂O (215 mg, 1.52 mmol, 187 μL) in PhMe (24 mL) was degassed and purged with N₂ 3 times. The mixture was stirred at 110° C. for 5 h. The reaction mixture was concentrated under reduced pressure, diluted with DCM (50 mL) and washed with water (100 mL). The combined organic layers were dried over Na₂SO₄, filtered and concentrated under reduced pressure. The residue was purified by prep-HPLC to give (2S,5S, 8S, 11S)-1,4,7,10-tetrabenzyl-2,5,8,11-tetraethyl-1,4,7,10-tetrazacyclododecane (6.73 g, 10.4 mmol, 15% yield) as a white solid; ¹H NMR (400 MHz, CHLOROFORM-d) δ ppm 0.98 (12H, t, J=7.3 Hz), 1.17-1.34(4H, m), 1.62-1.82 (4H, m), 1.90-2.14(4H, m), 2.91-3.17 (12H, m), 3.68(4H, d, J=139 Hz), 7.13-7.26 (10H, m), 7.27-7.35 (10H, m). MS (ESI) m/z: 645.4 (M+H)⁺. Rₜ=0.97 min.

### 37.4 Preparation of Compound 54

A mixture of (2S,5S, 8S,11S)-1,4,7,10-tetrabenzyl-2,5,8,11-tetraethyl-1,4,7,10-tetrazacyclododecane 53 (1.0 g, 1.55 mmol, 1 coq). Pd(OH)₂/C (571 mg, 0.41 mmol, 3.88 mL, 20% purity) and ammonium formate (500 mg, 7.93 mmol, 5.11 eq) in trifluoroethanol (8 mL) was degassed and purged with N₂ for 3 times. The mixture was stirred at 70° C. for 20 h. The reaction mixture was filtered and concentrated under reduced pressure to give crude (25,55,85, 11 S)-2,5,8,11-tetraethyl-1,4,7,10-tetrazacyclododecane (0.98 g) as a white solid; 1H NMR (400 MHz, CHLOROFORM-d) δ ppm 0.93 (12H, t, J=7.5 Hz), 1.40 (4H, dquin, J=14.2, 7.2 Hz), 1.53-1.65 (4H, m), 2.53-2.71 (8H, m), 2.76-2.85 (4H, m), 4.32-4.91 (4H, m), MS (ESI) m/z: 284.2 (M+H)⁺, Rₜ=0.58 min.

### 37.5 Preparation of Compound 55

A mixture of (2S,5S, 8S,11S)-2,5,8,11-tetraethyl-1,4,7,10-tetrazacyclododecane 54 (0.45 g, 1.58 mmol, 1 eq), tert-butyl 2-bromoacetate (1.54 g, 7.91 mmol, 1.17 mL, 5 eq) and K₂CO₃ (2.19 g, 15.8 mmol, 10 eq) in CH₃CN (45 mL) was degassed and purged with N₂ 3 times. The mixture was stirred at 50° C. for 16 h.

The reaction was cooled to room temperature, filtered and concentrated under reduced pressure. The residue was dissolved in 2% HCl (60 ml) and extracted with EtOAc (30*2 mL). The pH of the aqueous phase was adjusted to pH-8 and extracted with DCM (30 mL k2). The combined organic phases were dried over Na₂SO₄, filtered and the filtrate concentrated under reduced pressure. The residue was purified by prep-HPLC to give tert-butyl 2-[(2S,5S, 8S, 11S)-4,7,10-tris(2-tort-butoxy-2-oxo-ethyl)-2,5,8,11-tetraethyl-1,4,7,10-tetrazacyclododec-1-yl]acetate (0.35 g, 472 μmol, 30% yield) as a white solid: 1H NMR (400 MHz, CHLOROFORM-d) δ ppm 1.48 (48H, br s), 1.75-2.29 (8H, m), 2.68-3.06 (5H, m), 3.09-3.47(5H, m), 3.65-4.06(6H, m), 4.38-4.65 (3H, m) 4.76-5.04 (1H, m). MS(ESI)ₙ/z: 741.5 (M+H)⁺, Rₜ=3.17 min.

### 37.6 Preparation of Compound 56

To a solution of HCl (6 M, 1.0 ml) was added tert-butyl 2-[(25,5S, 8S, 11 S)-4,7,10-tris(2-tort-butoxy-2-oxo-ethyl)-2,5,8,11-tetraethyl-1,4,7,10-tetrazacyclododec-1-yl]acetate 55 (0.30 g, 405 μmol, 1 eq) and the mixture stirred at 90° C. for 1 h under a N₂ atmosphere. The reaction mixture was concentrated under reduced pressure and the residue purified by prep-HPLC to give 2-[(2S,5S, 8S, 11S)-4,7,10-tris(carboxymethyl)-2,5,8,11-tetraethyl-1,4,7,10-tetrazacyclododec-1-yl]acetic acid (173 mg, 335 μmol, 83% yield) as a white solid: ¹H NMR (400 MHz. D₂O) δ ppm 0.75-1.12 (12H, m), 1.19-1.52 (4H, m), 1.94(4H, br dd, J=12.7, 7.0 Hz), 2.72-2.96(3H, m), 3.17(5H, br d, J=17.4 Hz), 3.34-3.69(5H, m), 3.73-3.91 (4H, m), 4.08 (3H, br d, J=16.6 Hz). MS (ESI) m/z: 859.2 (M+H)Rₜ=0.85 min.

### 37.7 Preparation of Compound CHL-005

To a solution of 2-[(2S,5S, 8S, 11S)-4,7,10-tris(carboxymethyl)-2.5,8,11-tetraethyl-1,4,7,10-tetrazacyclododec-1-yl]acetic acid 56 (50 mg, 97 μmol, 2 eq) and 2-[(2S)-4-[2-[[(2S)-1-(4-aminobutyl)pyrrolidin-2-yl]methoxy]-7-(8-chloro-1-naphthyl)-6,8-dihydro-5H-pyrido[3,4-d]pyrimidin-4-yl]-1-prop-2-enoyl-piperazin-2-yl]acetonitrile (31 mg, 48.4 μmol, 1 eq) in DMF (1 mL) was added DIEA (125 mg, 968 μmol, 169 μL, 20 eq), EDCI (41 mg, 213 μmol, 4.4 eq) and HOBt (16 mg, 116 μmol, 2.4 eq) at 0° C. The reaction mixture was stirred at 40° C. for 16 h and concentrated under reduced pressure. The residue was purified by prep-HPLC to give 2-[(2S,5S,8S,11S)-7,10-bis(carboxymethyl)-4-[2-[4-[(251-2-[[7-(8-chloro-1-naphthyl)-4-[(3S)-3-(cyanomethyl)-4-prop-2-enoyl-piperazin-1-yl]-6,8 dihydro-5H-pyrido[3,4-d]pyrimidin-2-yl]oxymethyl]pyrrolidin-1-yl]butylamino]-2-oxo-ethyl]-2,5,8,11-tetraethyl-1,4,7,10-tetrazacyclododec-1-yl]acetic acid (19 mg, 16.7 μmol, 34% yield) as a yellow solid; ¹H NMR (400 MHz, methanol-d4) δ ppm 0.94-1.16 (12H, m), 1.20-1.51 (5H, m), 1.53-1.67 (2H, m), 1.73-2.28 (10H, m), 2.32-2.43 (1H, m), 2.69-3.00 (6H, m), 3.03-3.25 (10H, m), 3.36-3.85 (13H, m), 3.85-3.85(1H, m), 3.89-4.17(6H, m), 4.20-4.42(3H, m), 4.47-4.83(4H, m), 5.85 (1H, br d, J=10.1 Hz), 6.31 (1H, br d, J=16.5 Hz), 6.68-7.01 (1H, m), 7.33-7.45(2H, m), 7.48-7.60 (2H, m), 7.72 (1H, br d, J=8.1 Hz), 7.86 (1H, d, J=8.1 Hz). MS (ESI) m/z: 1140.6 (M+H)⁺. Rₜ=2.16 min.

### 37.8 Preparation of Compound Lu-CHL-005

To a solution of 2-[(2S,5S, 8S, 11S)-7,10-bis(carboxymethyl)-4-[2-[4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2-yl]oxymethyl]pyrrolidin-1-yl]butylamino]-2-oxo-ethyl]-2,5,8,11-tetraethyl-1,4,7,10-tetrazacyclododec-1-yl]acetic acid (50 mg, 43.8 limo, 1 eq) in NaOAc (1 mL) was added Lu(NO₃)₃ (90.2 mg, 438 μmol, 10 eq). The mixture was stirred at 80° C. for 1 h. The mixture was concentrated under reduced pressure and the residue purified by prep-HPLC to give N-[4-[(2S)-2-[[7-(8-chloro-1-naphthyl)-4-[(3S)-3-(cyanomethyl)-4-prop-2-enoyl-piperazin-1-yl]-6,8-dihydro-5H-pyrido[3,4-d]pyrimidin-2yl]oxymethyl]pyrrolidin-1-yl]butyL]1J-2-[(6S,9S, 12S, 21 S)-6,9,12,21-tetraethyl-3,16,19-trioxo-2,17,18-trioxa-5,8,11,14-tetraza-17,3-lutetatricyclo[9.6.3,25,14]docosan-8-yl]acetamide (15 mg, 11.5 μmol, 26% yield) as a white solid: ¹H NMR (4(K) MHz, METHANOL-d₄) δ 0.87-1.07 (13H, m), 1.16-1.48 (5H, m), 1.66-2.24(12H, m), 2.54 (6H, br d, J=13.9 Hz), 2.80 (5H, br d, J=9.3 Hz), 2.93-3.18 (8H, m), 3.42-3.66 (10H, m), 3.68-3.97 (6H, m), 3.99-4.21 (2H, m), 4.29-4.55 (3H, m), 4.59-4.76 (2H, m), 4.94-5.17 (1H, m), 5.85(1H, br d, J=10.0 Hz), 6.30(1H, br d, J=16.9 Hz), 6.76-6.94 (1H, m), 7.33-7.43 (2H, m), 7.48-7.60 (2H, m), 7.72 (1H, d, J=8.2 Hz), 7.85 (1H, d, J=8.2 Hz). MS (ESI) m/z: 1312.5, Rₜ=2.19 min.

### A35. Synthesis of CHL-006 and Lu-CHL-006

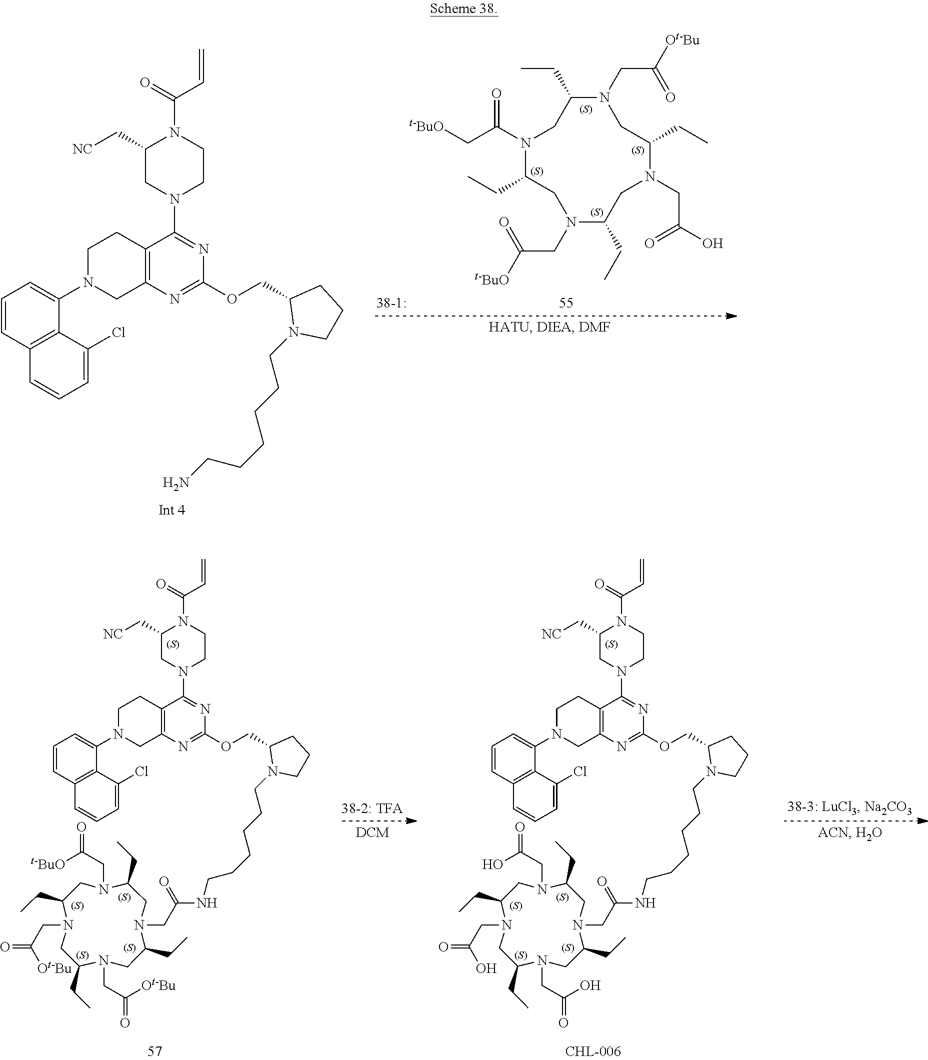

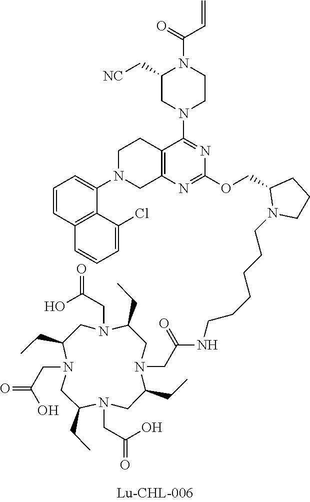 [SMILES: C=CC(=O)N1CCN(c2nc(OC[C@@H]3CCCN3CCCCCCNC(=O)CN3C[C@H](CC)N(CC(=O)O)C[C@H](CC)N(CC(=O)O)C[C@H](CC)N(CC(=O)O)C[C@@H]3CC)nc3c2CCN(c2cccc4cccc(Cl)c24)C3)C[C@@H]1CC#N]

### 38 Preparation of Compound CHL-006 and Lu-CHL-006

CHL-006 and Lu-CHL-006 are synthesized by following a similar procedure as depicted above in Scheme 37 for CHL-005 and Lu-CHL-005.

### Example A36: Synthesis of 177-Lutetium Chelated Radiopharmaceutical Conjugates

Conjugates of Table 5A and 5C are synthesized according to the same methods described in Example A311-A35.

General procedure for ¹⁷⁷Lu-labeling [¹⁷⁷Lu]LuCl₃ in HCl (50 MBq) is added to a mixture of a conjugate of Table 5A or 5C (1 nmol) in NaOAc buffer (5% EtOH, 0.25 M, pH 5.0-5.5, total volume 100-120 μL) in a 1.8 mL Eppendorf tube. The resulting mixture is heated at 80-85° C. in a thermal mixer at a shaking speed of 600 rpm for 15-30 min. If necessary, the mixture is purified using a C₈ column. Radiochemical purity is determined by radio-RP-HPLC and iTLC.

Accordingly, conjugates of 5B and 5D are synthesized according to Example A36.

### Example A37: Synthesis of 225-Actinium Chelated Conjugates

Conjugates of Table 5A and 5C are synthesized according to the same methods described in Example A30-A35

General procedure for ²²⁵Ac-labeling [²²⁵Ac]Ac(NO₃)₃ in 1 mM HCl (50 kBq) is added to a mixture of a conjugate of Table 5A or 5C (1 nmol) in NaOAc buffer (100 μL, 0.4 M. pH 5.5-6.5) in a 1.8 mL Eppendorf tube. The resulting mixture is heated at 80-1(a) ° C. in a thermal mixer at a shaking speed of 500 rpm for 15-30 min. Radiochemical purity is determined by iTLC.

Accordingly, conjugates of 5B and 5D are synthesized according to Example A37.

### Example A38. Exemplary Procedure for Synthesizing Covalently Modified KRAS Protein

A compound selected from Table 4A, Table 4B, Table 4C. Table 4D. Table 5A, Table 5B, Table 5C, and Table 5D is administered to a subject having mutated KRAS protein, including G12C according to SEQ ID NO 1 or SEQ ID NO: 2. The compound covalently binds with a KRAS protein in vivo at residue G12C, thereby generating a modified KRAS protein.

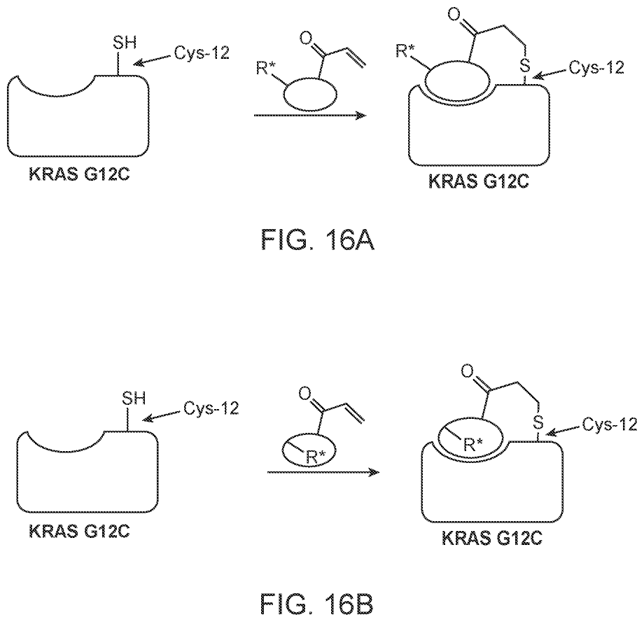

A modified KRAS protein is illustrated in FIG. 16A and FIG. 16B, as an example. The compound in FIG. 16A or FIG. 16B can be any radiolabeled compound described herein that contains an electrophilic functional group

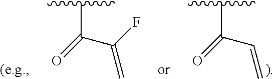

FIG. 16A or FIG. 16B also illustrates that the electrophilic functional group reacts with a cysteine residue of the KRAS protein.

### B: Biological Assays

### Example B1: KRAS G12C Nucleotide Exchange Assay

A KRAS (G12C) Nucleotide Exchange Assay kit was used to evaluate compound antagonistic binding. The assay kit (BPS Bioscience. Catalog #79859) utilizes KRAS (G12C) labeled with BODIPY-GDP to determine if compounds can affect the nucleotide exchange (GDP to GTP) in KRAS signaling, Compounds were typically characterized using both protocols: fixed GTP concentrations and fixed inhibitor concentrations.

### Fixed GTP Concentration Characterization

All reagents were prepared following manufacturer's protocol. 5 μL BODIPY-GDP and 10 μL of KRAS buffer were added to each well. A 3-fold serial dilution of compound in 5% DMSO was prepared. Typically, compounds were tested in the range of 0 μM to 50 μM, but the range could be modified depending on the characterization needs. 5 μL of diluted compound was added to the wells and the plate was centrifuged briefly to ensure all components were mixed before incubation at ambient temperature. After incubation for 2 hours, 10 μM GTP and 25 mM EDTA were mixed at a 1:1 ratio and 5 μL was added to the wells. The plate was then incubated for 1 hour at ambient temperature. After incubation, fluorescence was measured at Ex_{470cm}/Em_{525cm}.

Data was analyzed by plotting fluorescence vs GTP concentration for control wells and compound wells. IC₅₀ values for compounds of the present disclosure are provided in Table 7 below.

TABLE 7

| Compound | IC₅₀ (μM) |
|---|---|
| Ex-2 | B |
| Ex-3 | B |
| Ex-5 | B |
| Ex-9 | B |
| Lu-CHL-001 | B |
| Lu-CHL-002 | B |
| Lu-CHL-003 | A |

A is <1 μM; B is ≥1 μM and <10 μM; C is ≥10 μM.

### Fixed Inhibitor Concentration Characterization

All reagents are prepared following manufacturer's protocol. 5 μL of compound is added to each test well. Typically, 10 μM of compound is used, but the concentration could be modified depending on characterization needs. For both test wells and control wells, 10 μL of KRAS buffer and 5 μL BODIFY-GDP are added. 5 μL of compound buffer (e.g, 5% DMSO) is added to control wells. The plate is briefly centrifuged to ensure all reagents are mixed before incubation at ambient temperature. GTP reagent is serially diluted 0 mM to 1 mM in water. After 2 hours, 2.5 μL of the prepared GTP is add to all wells along with 2.5 μL EDTA at 25 mM. The plate is briefly centrifuged to mix all reagents and incubated at ambient temperature for 1 hour. After incubation, fluorescence is measured at Ex_{470cm}/Em_{525cm}.

Data is analyzed by plotting fluorescence vs compound concentration. EC₅₀ is calculated as the compound concentration that elicited 50% fluorescence.

### Example B2: Phosphorylated ERK1/ERK2 ELISA

An ERK phosphorylation ELISA kit was used to characterize compounds for KRAS-based cell activity. The sandwich ELISA (RnD Systems. Catalog #DYC1018B) measures human ERK1 that is dually phosphorylated at T202/Y204 and ERK2. Cell lysates from cells before and after treatment with compounds were prepared using kit manufacturer protocols.

All reagents were prepared following the kit's protocol. The wells of a 96-well microtiter plate were coated with 100 μL of 8.0 μg/mL of capture antibody in PBS. The plate was sealed and left to incubate overnight at ambient temperature. After incubation, the wells were washed with 400 μL of Wash Buffer for a total of 3 washes. Wells were then blocked by adding 300 μL of Block Buffer to each well and incubated at ambient temperature for 1-2 hours. After removal of the Block Buffer by aspirating and washing, 100 μL of standards and samples were added to the plate in duplicates and incubated for 2 hours at ambient temperature. The aspiration and wash steps were repeated to remove unbound standard and samples and 100 μL of 400 ng/mL detection antibody was applied to each well. After 2 hours at ambient temperature, detection antibody was removed by washing and 100 μL of Streptavidin-HRP was added to each well. Incubation of Streptavidin-HRP was at ambient temperature for 20 minutes, after which Streptavidin-HRP was removed by washing, 100 μL of Substrate Solution was added to each well and incubated for 20 minutes before 50 μL of Stop Solution was added to each well.

The optical density of each well was immediately analyzed at 450 nm with a wavelength correction at 540 nm. The standards were plotted by averaging the optical density for each replicate and fitted with a four-parameter logistic curve fit. Concentrations of phosphorylated ERK in cell lysate samples were determined by extrapolation from the standard curve and then adjusted for any dilution factors. IC₅₀ values for compounds of the present disclosure are provided in Table 8 below.

TABLE 8

| Compound | IC₅₀ (nM) |
|---|---|
| Ex-1 | B |
| Ex-2 | B |
| Ex-3 | B |
| Ex-4 | A |
| Ex-5 | C |
| Ex-6 | A |
| Ex-8 | B |
| Ex-9 | A |
| Ex-10 | A |
| Ex-11 | A |
| Ex-12 | A |
| Ex-13 | A |
| Ex-14 | A |
| CHL-001 | D |
| Lu-CHL-001 | C |
| Lu-CHL-002 | C |
| Lu-CHL-003 | C |
| CHL-004 | C |
| CHL-005 | C |
| Lu-CHL-005 | B |

A is <100 nM; B is ≥100 nM and <1,000 nM; C is ≥1,000 nM and <10,000 nM; and D is ≥10,000 nM.

### Example B3: Inhibition of KRas G12C-Dependent Cell Growth

The cellular inhibition of KRas G12C by specific compounds are measured by the inhibition of growth of cells dependent on the KRas G12C mutation.

MiaPaca-2 (ATCC, CRL-1420). NCI-H358 (ATCC CRL -5807), A549 (ATCC CCL-185), and NCI-H1975 (CRL -5908) cell lines are cultured according to ATCC cell culture recommendations. Cells are plated in sterile 96-well plates at a concentration of 60.000 cells/well and allowed to attach for 12-18 hours. Diluted compounds are added to the cells with a final concentration of 0.5% DMSO, in 200 uL volume of media. The compounds and remaining media are left to culture for 72 hours. At the end of the 72 hour incubation time, the plates are removed from the incubator and left to equilibrate to room temperature for use in the Cell Titer Glo 2.0 Cell Viability Assay (Promega Catalog #G9241). All reagents are thawed and allowed to equilibrate to room temperature before use in the assay. Reagent preparation followed the manufacturer's protocol. After reagent dilution, 25 uL of the CTG reagent is added to each well of the 96 well plate and set to shake for 20 minutes at room temperature. The plate is read on a SpectraMax iD5 using the Softmax Pro 7 Software from Molecular Devices. The Cell Titer Glo Luminescence protocol is used, reading the plate at a wavelength of 595 nM.

Resulting OD values are normalized by subtracting the background values of wells that did not contain cells, and then normalized to 100% by using the DMSO-only treated well. Subsequent cell growth inhibition is calculated for the compound dilution curve and graphed in Graphpad Prism.

Disclosure of the present application is further illustrated in the following list of embodiments, which are given for illustration purposes only and are not intended to limit the disclosure in any way:

Embodiment 1: A conjugate comprising:
a) a targeting ligand that covalently binds to an intracellular KRAS protein, wherein the intracellular KRAS protein is mutated;
b) a linker; and
c) a metal chelator.

Embodiment 2: The conjugate of embodiment 1, further comprising a radionuclide, wherein the radionuclide is bound to the metal chelator.

Embodiment 3: The conjugate of embodiment 1 or 2, wherein the targeting ligand covalently binds to the mutated KRAS protein at residue G12C, and wherein residue position numbering of the KRAS protein is based on SEQ ID NO:1 as a reference sequence.

Embodiment 4: The conjugate of embodiment 3, wherein the targeting ligand irreversibly binds to the mutated KRAS protein at residue G12C.

Embodiment 5: The conjugate of any one of embodiments 1 to 4, wherein the targeting ligand comprises an electrophilic functional group.

Embodiment 6: The conjugate of embodiment 5, wherein the electrophilic functional group forms a covalent bond with a cysteine residue of the mutated protein.

Embodiment 7: The conjugate of embodiment 5 or 6, wherein the electrophilic functional group comprises a functional group selected from an ester, acrylamide, halo-acrylamide, enamide, chloroacetamide, acyl aside, acyl nitrile, aldehyde, ketone, alkyl halide, alkyl sulfonate, anhydride, aryl halides, boronic acid, boronate, carboxylic acid, hydrazide, carbodiimide, diazoalkane, epoxide, haloacetamide, halotriazane, imido ester, isocyanate, isothiocyanate, maleimide, phosphoramidite, silyl halide, sulfonate ester, sulfonyl halide. α,β-unsaturated ester, vinyl sulfone, and propargyl amide, each of which is optionally substituted.

Embodiment 8: The conjugate of any one of embodiments 5 to 7, wherein the electrophilic functional group comprises optionally substituted acrylamide, optionally substituted chloroacetamide, or a derivative thereof.

Embodiment 9: The conjugate of embodiment 8, wherein the electrophilic functional group comprises an acrylamide group, a 2-fluoroacrylamide group, or a 2-methyl acrylamide group.

Embodiment 10: The conjugate of embodiment 7, wherein the electrophilic functional group comprises a substituted enamide group selected from acrylamide, 2-fluoroacrylamide, methacrylamide, 2-methoxyacrylamide, (E)-4-fluorobut-2-enamide. (E)-4-methoxybut-2-enamide. (E)-4-(pyrrolidin-1-yl)but-2-enamide, and (E)-4-(piperidin-1-yl)but-2-enamide.

Embodiment 11: The conjugate of any one of embodiments 5 to 8, wherein the electrophilic functional group comprises a structure of Formula (Ia) or Formula (Ib);
wherein,

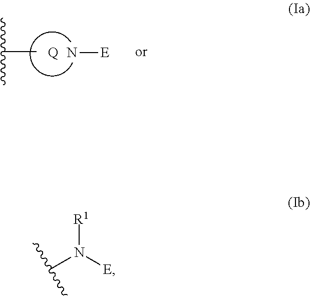

ring Q is a 3 to 10 membered heterocycloalkylene, wherein Q is optionally substituted;
R¹ is H, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; and
E represents a structure of Formula (Ic),
wherein,

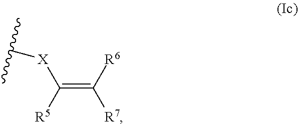

X is C(═O), P(═O)OR², C(═S), or S(═O)ₙ, where n is 1 or 2;
R² is H or substituted or unsubstituted C₁-C₆ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₇ cycloalkyl, or substituted or unsubstituted C₂-C₇ heterocycloalkyl; or R⁵ and R⁷ taken together form a bond;
R⁶ is H, halogen, —CN, C₁-C₆ alkyl, C₁-C₆heteroalkyl, heteroaryl, aryl, alkylaminylalkyl, dialkylaminylalkyl, cycloalkyl or heterocycloalkyl, each of which is optionally substituted.

Embodiment 12: The conjugate of any one of embodiments 5 to 8, wherein the electrophilic functional group comprises a structure of Formula (Id).

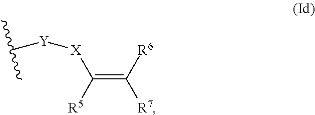

wherein,
X is C(═O), OC(═O), NR^{a}C(═O), P(═O)OR², C(═S). S(═O)ₙ, OS(O)ₙ, NR²S(═O)ₙ, wherein n is 1 or 2;
Y is an alkylene, heteroalkylene, cycloalkylene, heterocycloalkylene, arylene, heteroarylene, or a bond, wherein each of the alkylene, heteroalkylene, cycloalkylene, heterocycloalkylene, arylene, or heteroarylene is optionally substituted;
each R² is independently H or substituted or unsubstituted C₁-C₃ alkyl;
R⁵ and R⁷ are each independently selected from H, cyano, halogen, substituted or unsubstituted C₁-C₆ alkyl, substituted or unsubstituted C₁-C₆ heteroalkyl, substituted or unsubstituted C₃—C-cycloalkyl, or substituted or unsubstituted C₂-C₆ heterocycloalkyl; or R⁵ and R⁷ taken together form a bond; and
R⁶ is H, halogen, —CN, C₁-C₆ alkyl, C₁-C₆heteroalkyl, heteroaryl, aryl, alkylaminylalkyl, dialkylaminylalkyl, cycloalkyl or heterocycloalkyl, each of which is optionally substituted.

Embodiment 13: The conjugate of embodiment 11 or 12, wherein X is C(═O).

Embodiment 14: The conjugate of embodiment 12 wherein X is NR²C(═O).

Embodiment 15: The conjugate of any one of embodiments 11 to 14, wherein R⁵ is H, halogen, methyl, or —OMe.

Embodiment 16: The conjugate of any one of embodiments 11 to 15, wherein R⁵ is H.

Embodiment 17: The conjugate of any one of embodiments 11 to 16, wherein R⁶ is H.

Embodiment 18: The conjugate of any one of embodiments 11 to 17, wherein R⁷ is H or substituted or unsubstituted C₁-C₄ alkyl.

Embodiment 19: The conjugate of any one of embodiments 11 to 18, wherein R⁷ is H.

Embodiment 20: The conjugate of any one of embodiments 11 to 18, wherein R⁷ is —CH₂F, —CH₂OMe, or —CH₂-C₂-C₅ heterocycloalkyl.

Embodiment 21: The conjugate of embodiment 11, wherein E represents a structure of Formula (Ic) that is

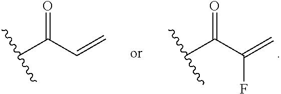

Embodiment 22: The conjugate of embodiment 11, wherein the structure of Formula (Ib) is

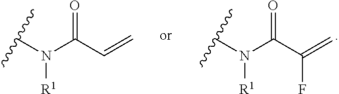

Embodiment 23: The conjugate of any one of embodiments 1 to 4, wherein the targeting ligand comprises a structure of Formula (III), or a salt, solvate, or derivative thereof,

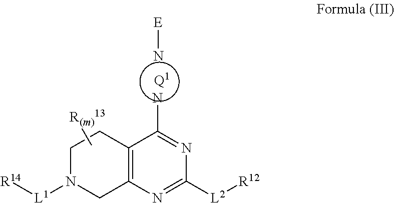

wherein
Q¹ is a 4-12 membered saturated or partially saturated monocyclic or bicyclic ring, wherein the monocyclic or bicyclic ring is optionally substituted;
L¹ is a bond, —C(O)—, or optionally substituted C₁-C₃ alkylene;
L² is a bond, O, S or NR¹⁵;
E is

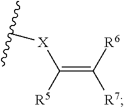

X is C(═O), P(═O)OR², C(═S), or S(═O)ₙ, where n is 1 or 2;
R² is H or substituted or unsubstituted C₁-C₃ alkyl;
R⁵ is hydrogen, cyano, halogen, alkyl, alkoxyl, heteroalkyl, C₃-C₆ cycloalkyl, or C₂-C₅ heterocycloalkyl, each of which is optionally substituted; and
R⁷ is hydrogen, cyano, halogen, alkyl, alkoxyl, heteroalkyl, C₃-C₇ cycloalkyl, C₂-C₇ heterocycloalkyl, heteroaryl, heteroarylalkyl, alkylaminylalkyl, or dialkylaminylalkyl, each of which is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, alkyl, alkoxyl, heteroalkyl, cycloalkyl, heterocycloalkyl, heteroaryl, heteroarylalkyl, alkylaminylalkyl, or dialkylaminylalkyl, each of which is optionally substituted;
R¹² is hydrogen, alkyl, heteroalkyl, -L³-alkylaminyl, -L³-dialkylaminyl, -L³—NR¹⁵R^{15′}, heterocycloalkyl, -L³-heterocycloalkyl, cycloalkyl, -L³-cycloalkyl, aryl, heteroaryl, -L³-aryl, or -L³-heteroaryl, wherein each of the L³, heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted;
each R¹³ is independently OH, halogen, substituted or unsubstituted C₁-C₆ alkyl, oxo, or substituted or unsubstituted C₁-C₆ heteroalkyl;
R¹⁴ is hydrogen, cycloalkyl, heterocycloalkyl, aryl, aralkyl, or heteroaryl, wherein each of the cycloalkyl, heterocycloalkyl, aryl, aralkyl, and heteroaryl is optionally substituted;
each R¹⁵ is independently hydrogen or C₁-C₃ alkyl;
each R^{15′} is independently hydrogen, acyl, C₁-C₃ alkyl, heteroalkyl or hydroxyalkyl; and
m is 0, 1, or 2; and
wherein the structure of Formula (III) is attached to the rest of the conjugate at any suitable position.

Embodiment 24: The conjugate of embodiment 23, wherein the targeting ligand is attached to the linker via the R¹² group.

Embodiment 25: The conjugate of embodiment 23 or 24, wherein R⁵ is hydrogen, cyano, halogen, C₁-C₆ alkyl, C₁-C₆ alkoxyl, C₁-C₆ heteroalkyl, C₃-C₆ cycloalkyl, or C₂-C₇ heterocycloalkyl, each of which is optionally substituted.

Embodiment 26: The conjugate of any one of embodiments 23 to 25, wherein R⁵ is hydrogen or a C₁-C₃ alkyl optionally substituted by one or more hydroxyl and/or halogen.

Embodiment 27: The conjugate of any one of embodiments 23 to 25, wherein R⁵ is a halogen.

Embodiment 28: The conjugate of any one of embodiments 23 to 25, wherein R⁵ is a hydrogen.

Embodiment 29: The conjugate of any one of embodiments 23 to 25, wherein R⁵ is C₁-C₆ heteroalkyl.

Embodiment 30: The conjugate of embodiment 29, wherein R⁵ is —C(O)NR¹⁵R^{15′}.

Embodiment 31: The conjugate of any one of embodiments 23 to 30, wherein R⁷ is hydrogen, cyano, halogen, C₁-C₆ alkyl C₁-C₆ alkoxyl, C₁-C₆ heteroalkyl, each of which is optionally substituted.

Embodiment 32: The conjugate of any one of embodiments 23 to 31, wherein R⁷ is H.

Embodiment 33: The conjugate of any one of embodiments 23 to 31, wherein R⁷ is C₁-C₆ heteroalkyl selected from —NHC(O)—C₁-C₃alkyl and —CH₂NHC(O)—C₁-C₃alkyl.

Embodiment 34: The conjugate of embodiment 23, wherein R⁵ and R⁷ taken together with the carbon atoms to which they are attached form a 5-8 membered partially saturated cycloalkyl, wherein the cycloalkyl is optionally substituted with one or more R¹⁷, wherein each R¹⁷ is independently halogen, hydroxyl, C₁-C₆ alkyl, cycloalkyl, alkoxy, haloalkyl, amino, cyano, heteroalkyl, hydroxyalkyl, —O-haloalkyl, or —S-haloalkyl.

Embodiment 35: The conjugate of any one of embodiments 23 to 34, wherein R⁶ is hydrogen, cyano, halogen, C₁-C₆ alkyl, C₁-C₆ alkoxyl, C₁-C₆ heteroalkyl, C₃-C₆ cycloalkyl, or C₂-C₆ heterocycloalkyl, each of which is optionally substituted.

Embodiment 36: The conjugate of any one of embodiments 23 to 35, wherein R⁶ is hydrogen.

Embodiment 37: The conjugate of any one of embodiments 23 to 35, wherein R⁶ is C₁—C heteroalkyl selected from —NHC(O)—C₁-C₃alkyl and —CH₂NHC(O)—C₁-C₃alkyl.

Embodiment 38: The conjugate of any one of embodiments 23 to 37, wherein Q¹ is optionally substituted with one or more R¹⁸, wherein R¹⁸ is oxo, C₁-C₆ alkyl, C₂-C₆ alkenyl, C₁-C₆ alkynyl, C₁-C₆ heteroalkyl, cyan, —C(O)OR¹⁵, —C(O)N(R¹⁵)(R^{15′}), —N(R¹⁵)(R^{15′}), wherein the alkyl, alkenyl, alkynyl, or heteroalkyl is optionally substituted.

Embodiment 39: The conjugate of any one of embodiments 23 to 38, wherein Q¹ is a 6 membered monocyclic ring, where in the monocyclic ring is optionally substituted with one or more R¹⁸, wherein R¹⁸ is methyl, —CH₂CN, carbonyl, hydroxyl, carboxyl, C(O)OR¹⁵.

Embodiment 40: The conjugate of any one of embodiments 23 to 39, wherein R¹⁹ is hydrogen, cycloalkyl, heterocycloalkyl, aryl, aralkyl, or heteroaryl, wherein each of the cycloalkyl, heterocycloalkyl, an 1, aralkyl, and heteroaryl is optionally substituted with one or more R¹⁶ wherein each R¹⁶ is independently halogen, hydroxyl, C₁-C₆ alkyl, cycloalkyl, alkoxy, acetyl, carboxyl. —C(O)OR¹⁵, haloalkyl, amino, cyano, heteroalkyl, hydroxyalkyl, —O-haloalkyl, or —S-haloalkyl.

Embodiment 41: The conjugate of any one of embodiments 23 to 40, wherein R¹⁴ is aryl or heteroaryl, optionally substituted with one or more R¹⁶, wherein each R¹⁶ is independently halogen, hydroxyl, C₁-C₃ alkyl, alkoxy, haloalkyl, amino, or cyano.

Embodiment 42: The conjugate of any one of embodiments 23 to 40, wherein R¹⁴ is napthyl optionally substituted with one or more R¹⁶, wherein each R¹⁶ is independently D, halogen, —CN. —NH₂, —NH(alkyl), —N(alkyl)₂, —OH, oxo, —CO₂H, —CO₂alkyl, —C(═O)NH₂, —C(═O)NH(alkyl). —C(═O)N(alkyl)₂, —S(═O)₂NH₂, —S(═O), NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

Embodiment 43: The conjugate of embodiment 42, wherein each R¹⁶ is independently oxo, halogen, —CN, —NH₂, —NH(CH₃), —N(CH₃)₂, —OH, —CO₂H, —CO₂(C₁-C₄alkyl), —OCO(C₁-C₄alkyl), —C(═O)NH₂, —C(═O)NH(C₁-C₄ alkyl), —C(═O)N(C)-C₄alkyl)₂, —S(═O)₂NH₂, —S(O)₂NH(C)—C₄ alkyl), —S(O)₂N(C₁-C₄alkyl)₂, C₁-C₄alkyl, C₃-C₆cycloalkyl, C₁-C₄fluoroalkyl, C₁-C₆ heteroalkyl, C₁-C₄alkoxy, C₁-C₄fluoroalkoxy, —SC₁-C₄alkyl, —S(═O)C₁-C₄alkyl, or —S(═O)₂(C₁-C₄ alkyl).

Embodiment 44: The conjugate of any one of embodiments 23 to 43, wherein R¹² is hydrogen, alkyl, heteroalkyl, -L³-alkylaminyl, -L³-dialkylaminyl, -L³—NR¹⁵R^{15′}, heterocycloalkyl, -L³-heterocycloalkyl, cycloalkyl, -L³-cycloalkyl, aryl, heteroaryl, -L³-aryl, or -L³-heteroaryl, wherein each of the L³, heterocycloalkyl, cycloalkyl, aryl, heteroaryl, alkyl or heteroalkyl is optionally substituted with one or more R¹⁹,
wherein each R¹⁹ is independently hydrogen, oxo, acyl, hydroxyl, hydroxyalkyl, cyan, halogen, alkyl, aralkyl, heteroalkyl, cycloalkyl, heterocycloalkyl, alkoxy, dialkylaminyl, dialkylamidoalkyl, or dialkylaminylalkyl, wherein the alkyl, aralkyl, heteroalkyl, cycloalkyl, or heterocycloalkyl is optionally substituted.

Embodiment 45: The conjugate of any one of embodiments 23 to 44, wherein R¹² is alkyl, heteroalkyl, -L³-alkylaminyl, -L³-dialkylaminyl. -L³—NR¹⁵R^{15′}, heterocycloalkyl, -L³-heterocycloalkyl, cycloalkyl, or -L³-cycloalkyl, wherein each of the L³, heterocycloalkyl, cycloalkyl, alkyl, or heteroalkyl is optionally substituted with one or more R¹⁹.

Embodiment 46: The conjugate of any one of embodiments 23 to 45, wherein L³ is C₁-C₄ alkylene or C₁-C₄ heteroalkylene, each of which is optionally substituted with one or more R¹⁹.

Embodiment 47: The conjugate of any one of embodiments 44 to 46, wherein R¹⁹ is independently hydrogen, oxo, acyl, hydroxyl, hydroxyalkyl, cyano, halogen, or C₁-C₃ alkyl.

Embodiment 48: The conjugate of any one of embodiments 23 to 47, wherein X is C(═O).

Embodiment 49: The conjugate of any one of embodiments 23 to 48, wherein L¹ is a bond.

Embodiment 50: The conjugate of any one of embodiments 23 to 49, wherein L² is a bond, O, S or NR¹⁵.

Embodiment 51: The conjugate of any one of embodiments 23 to 50, wherein each R¹³ is independently OH, halogen, or C₁-C₃ alkyl.

Embodiment 52: The conjugate of any one of embodiments 23 to 51, wherein m is 0 or 1.

Embodiment 53: The conjugate of any one of embodiments 1 to 4 or 23, wherein the targeting ligand comprises a structure of

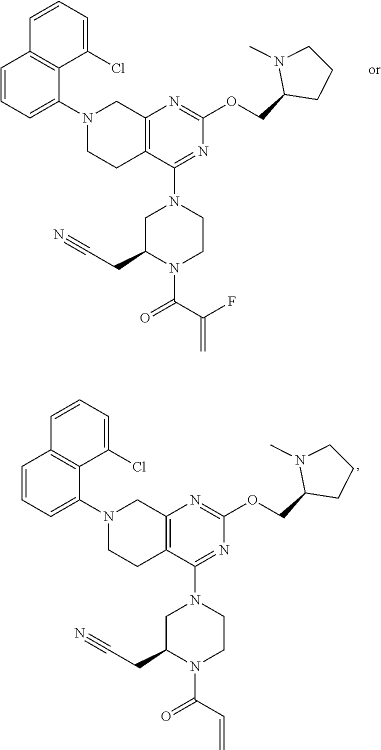 [SMILES: C=C(F)C(=O)N1CCN(c2nc(OC[C@@H]3CCCN3C)nc3c2CCN(c2cccc4cccc(Cl)c24)C3)C[C@@H]1CC#N ; C=CC(=O)N1CCN(c2nc(OC[C@@H]3CCCN3C)nc3c2CCN(c2cccc4cccc(Cl)c24)C3)C[C@@H]1CC#N]

wherein the structure is attached to the rest of the conjugate at any suitable position.

Embodiment 54: The conjugate of any one of embodiments 1 to 4, wherein the targeting ligand comprises a structure of Formula (IV), or a salt, solvate, or derivative thereof,

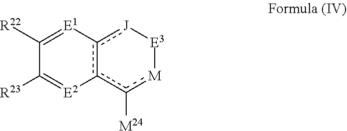

wherein
E¹ and E² are each independently N or CR²¹;
J is N, NR³⁰, or CR³⁰;
M is N, NR³³, or CR³³;
[symbol] is a single or double bond as necessary to give every atom its normal valence;
R²¹ is independently H, hydroxyl, cyano, halogen, alkyl, alkoxyl, or heteroalkyl, each of which is optionally substituted;
R²² is hydrogen, halogen, alkyl, alkenyl, alkynyl, OR′. N(R^{10′})₂, cycloalkyl, heterocycloalkyl, aryl, heteroaryl, each of which is optionally substituted, and each R′ is independently H, alkyl, cycloalkyl, heterocycloalkyl, alkenyl, alkynyl, aryl, or heteroaryl, each of which is optionally substituted, or two R′ substituents, together with the nitrogen atom to which they are attached, form a 3-7-membered ring;
   R²³ is hydrogen, halogen, alkyl, alkoxy, cycloalkyl, heterocycloalkyl, alkenyl, alkynyl, aryl, or heteroaryl, each of which is optionally substituted;
   R²⁴ is

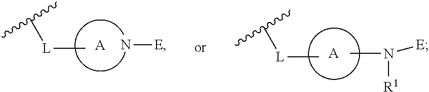

ring A is a substituted or unsubstituted 4-7 membered monocyclic ring or a substituted or unsubstituted 6-11 membered bicyclic, bridged fused, or spiro ring;
R¹ is H, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl;
L is a bond, alkylene, heteroalkylene, S, O, or NH;
E represents a structure of Formula (Ic).

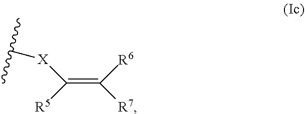

wherein,
X is C(═O), P(═O)OR², C(═S), or S(O)ₙ, where n is 1 or 2;
R² is H or substituted or unsubstituted C₁-C₃ alkyl;
R⁵ is hydrogen, cyano, halogen, alkyl, alkoxyl, heteroalkyl, C₃-C₆ cycloalkyl, or C₂-C₅ heterocycloalkyl, each of which is optionally substituted, and
R⁷ is hydrogen, cyano, halogen, alkyl, alkoxyl, heteroalkyl, C₃-C₇ cycloalkyl, C₂-C₇ heterocycloalkyl, heteroaryl, heteroarylalkyl, alkylaminylalkyl, or dialkylaminylalkyl, each of which is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, alkyl, alkoxyl, heteroalkyl, cycloalkyl, heterocycloalkyl, aryl, heteroaryl, heteroarylalkyl, alkylaminylalkyl, or dialkylaminylalkyl, each of which is optionally substituted;
E³ is CR²⁸R²⁹, C═CR²⁸R²⁹, C═O, C═S, or C═NR²⁸;
R²⁸ and R²⁹ are each independently H, alkyl, hydroxy, alkoxy, cyano, nitro, or cycloalkyl, or R²⁸ and R²⁹ taken together with the carbon atom to which they are attached, can form a 3-6 membered ring;
R³⁰ is selected from halogen, cyan, alkyl, cycloalkyl, aryl, heteroalkyl, heterocycloalkyl, heteroaryl, alkylaryl, alkylcycloalkyl, alkylheterocycloalkyl, alkylheteroaryl, alkoxy, heteroalkylaryl, heteroalkylheteroaryl, heteroalkylcycloalkyl, heteroalkylheterocycloaklyl, each of which is optionally substituted; and
R³³ is alkyl, heteroalkyl, or cycloalkyl, each of which is optionally substituted;
wherein the structure of Formula (IV) is attached to the rest of the conjugate at any suitable position.

Embodiment 55: The conjugate of embodiment 54, wherein the targeting ligand is attached to the linker via group R²².

Embodiment 56: The conjugate of embodiment 54 or 55, wherein
E¹ is N or CR²¹;
E² is CR²¹;
J is NR³⁰; and
M is N.

Embodiment 57: The conjugate of any one of embodiments 54 to 56, wherein R²¹ is independently H, hydroxyl, cyano, halogen, C₁-C₆alkyl, C₁-C₆ haloalkyl, C₁-C₄alkoxyl, or C₁-C₄heteroalkyl.

Embodiment 58: The conjugate of any one of embodiments 54 to 57, wherein R²¹ is independently H, hydroxyl, cyano, halogen, or methyl.

Embodiment 59: The conjugate of any one of embodiments 54 to 58, wherein R²¹ is H.

Embodiment 60: The conjugate of any one of embodiments 54 to 59, wherein R²² is halogen, C₁-C₆alkyl, C₂-C₃alkenyl, C₂-C₃alkynyl, OR′, N(R^{10′})₂, C₃-C₆cycloalkyl, C₂-C₅heterocycloalkyl, C₆-C₁₄ aryl, C₂-C₁₄heteroaryl, each of which is optionally substituted, and each R′ is independently H, C₁-C₆alkyl, C₃-C₆cycloalkyl, C₂-C₅heterocycloalkyl, C₂-C₃alkenyl, C₂-C₃alkynyl, C₆-C₁₄aryl, C₂-C₁₄heteroaryl, each of which is optionally substituted, or two R′ substituents, together with the nitrogen atom to which they are attached, form a 3-7-membered ring.

Embodiment 61: The conjugate of any one of embodiments 54 to 60, wherein R²² is C₆-C₁₄aryl or C₂-C₁₄heteroaryl, each of which is optionally substituted.

Embodiment 62: The conjugate of any one of embodiments 54 to 61, wherein R²² is phenyl, optionally substituted with one or more C₁-C₃alkyl, halogen, and/or hydroxyl.

Embodiment 63: The conjugate of any one of embodiments 54 to 62, wherein R²³ is H, halogen, C₁-C₃alkyl, C₁-C₃alkoxy, C₃-C₆cycloalkyl, C₂-C₅heterocycloalkyl, C₂-C₃alkenyl, C₂-C₃alkynyl, C₆-C₁₄aryl, or C₂-C₁₄heteroaryl, each of which is optionally substituted.

Embodiment 64: The conjugate of any one of embodiments 54 to 63, wherein R²³ is halogen, C₁-C₃alkyl, C₁-C₃haloalkyl.

Embodiment 65: The conjugate of any one of embodiments 54 to 64, wherein R²³ is halogen.

Embodiment 66: The conjugate of any one of embodiments 54 to 65, wherein R²⁴ is

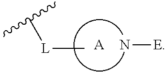

Embodiment 67: The conjugate of any one of embodiments 54 to 66, wherein ring A is a substituted or unsubstituted 4-7 membered monocyclic ring.

Embodiment 68: The conjugate of any one of embodiments 54 to 67, wherein ring A is substituted or unsubstituted 6 membered heterocyclic ring.

Embodiment 69: The conjugate of any one of embodiments 54 to 68, wherein ring A is piperazinyl substituted with halogen or C₁-C₃alkyl.

Embodiment 70: The conjugate of any one of embodiments 54 to 69, wherein L is a bond, C₁-C₃alkylene, S, O, or NH.

Embodiment 71: The conjugate of any one of embodiments 54 to 70, wherein L is a bond, CH₂, O, or NH.

Embodiment 72: The conjugate of any one of embodiments 54 to 71, wherein L is a bond.

Embodiment 73: The conjugate of any one of embodiments 54 to 72, wherein X is C(═O).

Embodiment 74: The conjugate of any one of embodiments 54 to 73, wherein R⁵ is hydrogen, cyano, halogen, C₁-C₆ alkyl, C₁-C₆, alkoxyl, C₁-C₆ heteroalkyl, C₃-C₆ cycloalkyl, or C₂-C₅ heterocycloalkyl, each of which is optionally substituted.

Embodiment 75: The conjugate of embodiment of any one of embodiments 54 to 74, wherein R⁵ is hydrogen or a C₁-C₃alkyl optionally substituted by one or more hydroxyl and/or halogen.

Embodiment 76: The conjugate of any one of embodiments 54 to 74, wherein R⁵ is a halogen.

Embodiment 77: The conjugate of any one of embodiments 54 to 74, wherein R⁵ is C₁-C₆ heteroalkyl.

Embodiment 78: The conjugate of any one of embodiments 54 to 75, wherein R⁵ is hydrogen.

Embodiment 79: The conjugate of any one of embodiments 54 to 78, wherein R⁷ is hydrogen, cyano, halogen, C₁-C₆ alkyl, C₁-C₆ alkoxyl, C₁-C₆ heteroalkyl, each of which is optionally substituted.

Embodiment 80: The conjugate of any one of embodiments 54 to 79, wherein R⁷ is hydrogen.

Embodiment 81: The conjugate of any one of embodiments 54 to 79, wherein R¹⁷ is C₁-C₆ heteroalkyl selected from —NHC(O)—C₁-C₃alkyl and —CH₂NHC(O)—C₁-C₃alkyl.

Embodiment 82: The conjugate of embodiment 54, wherein R⁵ and R⁷ taken together with the carbon atoms to which they are attached form a 5-8 membered partially saturated cycloalkyl, wherein the cycloalkyl is optionally substituted with one or more R¹⁷, wherein each R¹⁷ is independently halogen, hydroxyl, C₁-C₆ alkyl, cycloalkyl, alkoxy, haloalkyl, amino, cyano, heteroalkyl, hydroxyalkyl, —O-haloalkyl, or —S-haloalkyl.

Embodiment 83: The conjugate of any one of embodiments 54 to 82, wherein R⁶ is hydrogen, cyano, halogen, C₁-C₆ alkyl, C₁-C₆ alkoxyl, C₁-C₆ heteroalkyl, C₃-C₆ cycloalkyl, or C₂-C₆ heterocycloalkyl, each of which is optionally substitute.

Embodiment 84: The conjugate of any one of embodiments 54 to 83, wherein R⁶ is hydrogen.

Embodiment 85: The conjugate of any one of embodiments 54 to 83, wherein R⁶ is C₁-C₆ heteroalkyl selected from —NHC(O)—C₁-C₃alkyl and —CH₂NHC(O)—C₁-C₆alkyl.

Embodiment 86: The conjugate of any one of embodiments 54 to 85, wherein E³ is C═O, C═S, or C═NH.

Embodiment 87: The conjugate of any one of embodiments 54 to 86, wherein E is C═O.

Embodiment 88: The conjugate of any one of embodiments 54 to 87, wherein R³⁰ is halogen, cyano, C₁-C₈alkyl, C₁-C₁₄cycloalkyl, C₆-C₁₄aryl, C₁-C₁₄heteroalkyl, C₁-C₆heterocycloalkyl, C₂-C₁₄heteroaryl, C₁-C₃alkyl-C₆-C₁₄aryl, C₁-C₃alkyl-C₃-C₁₄cycloalkyl, C₁-C₃alkyl₂-C₁₄heterocycloalkyl, C₁-C₃alkyl-C₂-C₁₄heteroaryl, C₁-C₃alkoxy, C₀-C₃heteroalkyl-C₆-C₁₄aryl, C₀-C₃heteroalkyl-C₂-C₁₄heteroaryl, C₀-C₃heteroalkyl-C₃-C₆cycloalkyl, C₀-C₃heteroalkyl-C₂-C₁₄heterocycloalkyl, each of which is optionally substituted.

Embodiment 89: The conjugate of any one of embodiments 54 to 88, wherein R³⁰ is C₆-C₁₄aryl, optionally substituted with one or more of halogen, C₁-C₃alkyl, C₁-C₃alkoxyl, or cyano.

Embodiment 90: The conjugate of any one of embodiments 54 to 88, wherein R³⁰ is C₂-C₁₄heteroalkyl, optionally substituted with one or more of halogen, C₁-C₃alkyl, C₁-C₃alkoxyl, or cyano.

Embodiment 91: The conjugate of any one of embodiments 54 to 88, wherein R³⁰ is a 6-membered heteroaryl, optionally substituted with one or more of C₁-C₃alkyl.

Embodiment 92: The conjugate of any one of embodiments 54 to 91, wherein R³³ is C₁-C₆ alkyl, C₁-C₆haloalkyl, C₁-C₆alkylamine, or C₃-C₁₄cycloalkyl.

Embodiment 93: The conjugate of any one of embodiments 54 to 92, wherein R³³ is C₁-C₆ alkyl.

Embodiment 94: The of any one of embodiments 1 to 4 or wherein the targeting ligand is

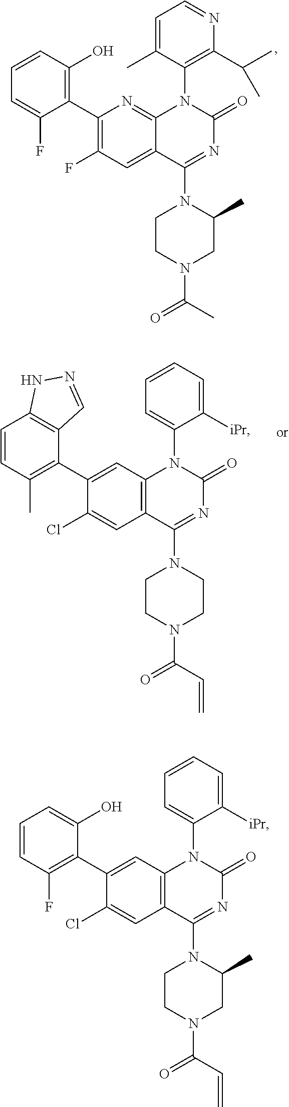

wherein the structure is attached to the rest of the conjugate at any suitable position.

Embodiment 95: The conjugate of any one of embodiments 1 to 94, wherein the metal chelator is selected from AAZTA. BAT, BAT-TM. Crown. Cyclen. DO2A. CB-DO2A. DO3A, H₃HP-DO3A. Oxo-DO3A, p-NH₂-Bn-Oxo-DO3A, DOTA, DOTA-3py, DOTA-PA, DOTA-GA, DOTA-4AMP. DOTA-2py, DOTA-1py, p-SCN-Bn-DOTA, CHX-A″-EDTA. MeO-DOTA-NCS EDTA, DOTAMAP, DOTAGA, DOTAGA-anhydride. DOTMA, DOTASA, DOTAM, DOTP, CB-Cyclam, TE2A. CB-TE2A. CB-TE2P. DM-TE2A. MM-TE2A, NOTA. NOTP. HEHA, HEHA-NCS, p-SCN-Bn-HEHA, DTPA. CHX-A″-DTPA, p-NH₂—Bn-CHX-A″-DTPA, p-SCN-DTPA, p-SCN-Bz-Mx-DTPA, 1B4M-DTPA-DTPA, p-SCN-Bn1B-DTPA, p-SCN-Bn-1B4M-DTPA, p-SCN-Bn-CHX-A″-DTPA. PEPA, p-SCN-Bn-PEPA, TETPA. DOTPA, DOTMP, DOTPM, t-Bu-calix[4]arene-tetracarboxylic acid, macropa, macropa-NCS, macropid, H₃L¹. H₃L⁴, H₂azapa. Hsdecapa, bispa², H₄pypa. H.4octapa, H₄CHXoctapa, p-SCN-Bn-H; octapa, p-SCN-Bn-H₄octapa, TTHA, p-NO₂-Bn-neunpa, H₄octox. H-macropa, H₂bispa², H₄phospa, H₆phospa, p-SCN-Bn-H₆phospa. TETA, p-NOrBn-TETA, TRAP. TRAP-Pr. TPA. HBED, SHBED, HBED-CC. (HBED-CC)TFP. DMSA, DMPS, DHLA, lipoic acid. TGA, BAL. Bis-thioseminarabacones, p-SCN-NOTA, nNOTA, NODAGA. CB-TE1A1P, 3P-C-NETA-NCS, 3p-C-DEPA, 3P-C-DEPA-NCS. TCMC, PCTA. NODIA-Me, TACN, pycupIAIB, pycup2A. THP. DEDPA, H₂DEDPA, p-SCN-Bn-H₂DEDPA, p-SCN-Bn-TCMC, motexafin, NTA. NOC, 3p-C-NETA, p-NH₂—Bn-TE3A. SarAr, DiAmSar. SarAr-NCS. AmBaSar. BaBaSar, TACN-TM, CP256. C-NE3TA, C-NE3TA-NCS, NODASA, NETA-monoamide, C-NETA, TACN-HSB. NOPO, BPCA, p-SCN-Bn-DRO, DRO-ChX-Mal, DFO. DFO-IAC, DFO-BAC, DiP-LICAM. EC. SBAD. BAPEN. TACHPYR. NEC-SP. L^{py}, L¹, L², L³, and EuK-106.

Embodiment 96: The conjugate of any one of embodiments 1 to 95, wherein the metal chelator is 2,2′,2″,2′″-((2S,5S,8S,11S)-2,5,8,11-tetramethyl-1.4,7,10-tetraazacyclododecane-1,4,7,10-tetrayl)tetraacetic acid.

Embodiment 97: The conjugate of any one of embodiments 1 to 95, wherein the metal chelator is 2,2′,2″,2′″4(2 S,5 S, 8S, 11 S)-2,5,8, 11-tetraethyl-1,4,7,10-tetraazacyclododecane-1,4,7,10-tetrayl)tetraacetic acid.

Embodiment 98: The conjugate of any one of embodiments 1 to 94, wherein the metal chelator is a chelator in FIG. 1 to FIG. 15.

Embodiment 99: The conjugate of any one of embodiments 1 to 94, wherein the metal chelator is DOTA.

Embodiment 100: The conjugate of any one of embodiments 2 to 99, wherein the radionuclide is astatine-211, astatine-217, actinium-225, americium-243, radium-223, lead-212, lead-203, copper-64, copper-67, copper-60, copper-61, copper-62, bismuth-212, bismuth-213, gallium-68, gallium-67, dysprosium-154, gadolinium-148, gadolinium-153, samarium-146, samarium-147, samarium-153, terbium-149, thorium-227, thorium-229, iron-59, yttrium-86, indium-111, holmium-166, technetium-94, technetium-99m, yttrium-90, lutetium-177, terbium-161, rhenium-186, rhenium-188, cobalt-55, scandium-43, scandium-44, scandium-47, dysprosium-166, fluorine-18, or iodine-131.

Embodiment 101: The conjugate of embodiment 100, wherein the radionuclide is lutetium-177, actinium-225, yttrium-90, bismuth-213, gallium-68, copper-64 or indium-111.

Embodiment 102: The conjugate of any one of embodiments 2 to 99, wherein the radionuclide is an alpha particle-emitting radionuclide.

Embodiment 103: The conjugate of embodiment 102, wherein the alpha particle-emitting radionuclide is actinium-225, astatine-211, thorium-227, or radium-223.

Embodiment 104: The conjugate of embodiment 102 or 103, wherein the alpha particle-emitting radionuclide is actinium-225.

Embodiment 105: The conjugate of any one of embodiments 2 to 99, wherein the radionuclide is a beta particle-emitting radionuclide.

Embodiment 106: The conjugate of embodiment 105, wherein the beta particle-emitting radionuclide is zirconium-89, yttrium-90, iodine-131, samarium-153, lutetium-177, or lead-212.

Embodiment 107: The conjugate of any one of embodiments 2 to 99, wherein the radionuclide is a gamma particle-emitting radionuclide.

Embodiment 108: The conjugate of embodiment 107, wherein the gamma particle-emitting radionuclide is indium-111.

Embodiment 109: The conjugate of any one of embodiments 1 to 108, wherein the linker covalently attaches the targeting ligand to the metal chelator.

Embodiment 110: The conjugate of embodiment 109, wherein the linker comprises substituted or unsubstituted C₁-C₆ alkylene or substituted or unsubstituted C₁-C₆ heteroalkylene.

Embodiment 111: The conjugate of embodiment 110, wherein the linker comprises propyl ethyl ether.

Embodiment 112: The conjugate of embodiment 109, wherein the linker comprises one or more amino acids.

Embodiment 113: The conjugate of any one of embodiments 1 to 108, wherein the conjugate has a structure of Formula (X).

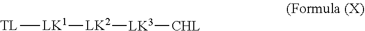

wherein,
TL represents the targeting ligand;
CHL represents the metal chelator, optionally bound to a radionuclide; and
each of LK¹, LK², and LK³ is independently selected from substituted or unsubstituted C₁-C₁₂ alkylene, substituted or unsubstituted C₁-C₁: heteroalkylene, substituted or unsubstituted C₂-C₁₂ alkenylene, substituted or unsubstituted C₁-C₁₂ alkynylene, —(CH₂CH₂O)ₙ, —(OCH₂CH₂)ₙ—, —O—, —S—, —S(O)—, —S(═O)₂—, —S(═O)(═NR^{LK})—, —C(═O)—, —C(═N—OR^{LK})—, —C(═O)O—, —OC(═O)—, —C(═O)C(═O)—, —C(═O)NR^{LK}—, —NR′ C(═O)—, —OC(═O)NR^{LK}—, —NR^{LK}S(═O)C—, (O)O—, —NR^{LK}C(═O)NR^{LK}—, —C(═O)NR^{LK}C(O)—, —S(O)₂NR^{LK}—, —NRS(═O)ₙ—, —NR^{LK}—, —N(OR^{LK})—, and a bond;
each R^{LK} is independently H or substituted or unsubstituted C₁-C₆alkyl;
m is an integer selected from 1 to 10; and
n is an integer selected from 1 to 20.

Embodiment 114: The conjugate of embodiment 113, wherein LK¹ is substituted or unsubstituted C₁-C₁₂ alkylene or substituted or unsubstituted C₁-C₁₂ heteroalkylene.

Embodiment 115: The conjugate of embodiment 113 or 114, wherein each of LK² and LK³ is independently a bond, C₁-C₆ alkylene, C₁-C₆ heteroalkylene, —(CH₂CH₂O)₁₋₃—, —(OCH₂CH₂)₁₋₃—, —O—, or —S—.

Embodiment 116: The conjugate of any one of embodiments 1 to 115, wherein the conjugate has an elimination half-life in a subject of about 0.1 to about 120 hours.

Embodiment 117: The conjugate of any one of embodiments 1 to 115, wherein the conjugate has an elimination half-life in a subject of about 10 minutes to about 20 hours.

Embodiment 118: The conjugate of any one of embodiments 1 to 115, wherein the conjugate has an elimination half-life in a subject of about 30 minutes to about 12 hours.

Embodiment 119: The conjugate of any one of embodiments 1 to 115, wherein the conjugate has an elimination half-life in a subject of about 5 minutes to about 6 hours.

Embodiment 120: The conjugate of any one of embodiments 1 to 115, wherein the conjugate has an elimination half-life in a subject of about 15 minutes to about 3 hours.

Embodiment 121: The conjugate of any one of embodiments 1 to 120, wherein the conjugate has a residence time of about 0.5 to 7 days in a tumor when administered to a subject having the tumor.

Embodiment 122: The conjugate of any one of embodiments 1 to 120, wherein the conjugate has a residence time of about 0.5 to 14 days in a tumor when administered to a subject having the tumor.

Embodiment 123: The conjugate of any one of embodiments 1 to 120, wherein the conjugate has a residence time of about 2 to 7 days in a tumor when administered to a subject having the tumor.

Embodiment 124: The conjugate of any one of embodiments 1 to 120, wherein the conjugate has a residence time of about 3 to 6 days in a tumor when administered to a subject having the tumor.

Embodiment 125: The conjugate of embodiment 1, wherein the conjugate has a structure listed in Tables 1B and 1D.

Embodiment 126: The conjugate of embodiment 1, wherein the conjugate has a structure listed in Tables 1A and 1C.

Embodiment 127: The conjugate of embodiment 126, further comprising a radionuclide selected from lutetium-177, actinium-225, yttrium-90, bismuth-213, gallium-68, copper-64 and indium-111 bound to the metal chelator.

Embodiment 128: A pharmaceutical composition comprising a conjugate of any one of embodiments 1 to 127, and a pharmaceutically acceptable excipient or carrier.

Embodiment 129: The pharmaceutical composition of embodiment 128, wherein the pharmaceutical composition is formulated for intravenous administration.

Embodiment 130: A method of treating cancer in a subject in need thereof, comprising administering to the subject a conjugate of any one of embodiments 1 to 127, or a pharmaceutical composition of embodiment 128 or 129.

Embodiment 131: The method of embodiment 130, wherein the cancer is KRAS G12C-associated cancer.

Embodiment 132: The method of embodiment 130 or 131, wherein the cancer is selected from the group consisting of Cardiac cancer; sarcoma (angiosarcoma, fibrosarcoma, rhabdomyosarcoma, liposarcoma), myxoma, rhabdomyoma, fibroma, lipoma and teratoma; Lung cancer; bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal cancer; esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract cancer; kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver cancer; hepatoma (hepatocellular carcinoma), cholangiocarcinoma, hepatoblastoma, angiosarcoma, hepatocellular adenoma, hemangioma; Biliary tract cancer; gall bladder carcinoma, ampullary carcinoma, cholangiocarcinoma; Bone cancer; osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system cancer; skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological cancer; uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic cancer; blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin cancer; malignant melanoma, basal cell carcinoma, squamous cell carcinoma, Kaposi's sarcoma, moles dysplastic nevi, lipoma, angioma, dermatofibroma, keloids, psoriasis; and Adrenal glands cancer; neuroblastoma.

Embodiment 133: The method of embodiment 130 or 131, wherein the cancer is non-small cell lung cancer.

Embodiment 134: A method of killing a cell harboring a G12C KRAS mutation, the method comprising contacting a cell harboring a G12C KRAS mutation with the conjugate of any one of embodiments 1 to 127, or a pharmaceutical composition of embodiment 128 or 129, thereby delivering a dose of radiation to the cell.

Embodiment 135: A method of delivering a radionuclide to a cell comprising administering the conjugate of any one of embodiments 1 to 127, or a pharmaceutical composition of embodiment 128 or 129.

Embodiment 136: The method of embodiment 135, wherein the conjugate irreversibly binds to an intracellular protein of the cell.

Embodiment 137: A method of diagnosing cancer patients harboring a G12C KRAS mutation comprising administering to a patient the conjugate of any one of embodiments 1 to 127, or a pharmaceutical composition of embodiment 128 or 129.

Embodiment 138: A method of imaging a cancer harboring a G12C KRAS mutation comprising administering to a patient the conjugate of any one of embodiments 1 to 127, or a pharmaceutical composition of embodiment 128 or 129.

Embodiment 139: The method of embodiment 137 or 138, further comprising measuring the concentration of the conjugate accumulated in the patient.

Embodiment 140: The method of any one of embodiments 137 to 139, further comprising measuring the amount of radiation emitted from the radionuclide.

Embodiment 141: The method of any one of embodiments 137 to 140, further comprising analyzing the elimination profile of the conjugate in the patient.

Embodiment 142: The method of any one of embodiments 137 to 141, further comprising measuring an elimination half-life of the conjugate in the patient.

Embodiment 143: A covalently modified KRAS protein comprising.
a KRAS protein comprising a glycine to cysteine amino acid substitution at residue 12, and
a radiolabeled compound comprising a covalently bonded radioisotope, wherein the radiolabeled compound is bonded to the KRAS protein at the cysteine residue 12 of the KRAS protein through a covalent bond, and
wherein residue position numbering of the KRAS protein is based on SEQ ID NO: 1 or SEQ ID NO:2 as a reference sequence.

Embodiment 144: The KRAS protein of embodiment 143, wherein the covalently bonded radioisotope is fluorine-18 (¹⁸F), iodine-131 (¹³¹I), iodine-123 (¹²³I), iodine-124 (¹²⁴I), iodine-125 (¹²⁵I), or astatine-211 (²¹¹At).

Embodiment 145: The KRAS protein of embodiment 143 or 144, wherein the covalently bonded radioisotope is ¹³¹I.

Embodiment 146: The KRAS protein of any one of embodiments 143 to 145, wherein the covalent bond is formed in vivo.

Embodiment 147: The KRAS protein of any one of embodiments 143 to 146, wherein the radiolabeled compound comprises an electrophilic functional group.

Embodiment 148: The KRAS protein of embodiment 147, wherein the covalent bond is formed between the electrophilic functional group and the cysteine residue 12 of the KRAS protein.

Embodiment 149: The KRAS protein of embodiment 147 or 148, wherein the electrophilic functional group comprises optionally substituted acrylamide or optionally substituted chloroacetamide.

Embodiment 150: The KRAS protein of embodiment 149, wherein the electrophilic functional group comprises an acrylamide group, a 2-fluoroacrylamide group, or a 2-methyl acrylamide group.

Embodiment 151: The KRAS protein of embodiment 147 or 148, wherein the electrophilic functional group comprises a structure of Formula (Ia):

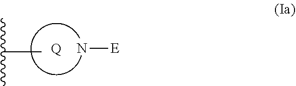

wherein,
ring Q is an optionally substituted 3 to 10 membered heterocycloalkyl; and
E has a structure of Formula (Ic),

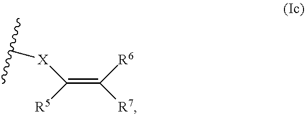

wherein,
X is C(═O), OC(═O), P(O)OR⁼, C(═S). S(═O), or OS(O);
n is 1 or 2;
R² is H or substituted or unsubstituted C, —C₁₋₃alkyl;
R⁵ and R⁷ are each independently selected from H, —CN, halogen, substituted or unsubstituted C, —C₆ alkyl, substituted or unsubstituted C₁-C₆ heteroalkyl, substituted or unsubstituted C₃-C₇ cycloalkyl, or substituted or unsubstituted C₂-C₇ heterocycloalkyl; or R⁵ and R⁷ taken together form a bond; and
R⁶ is H, halogen, —CN, C₁-C₆ alkyl, C₁-C₆ heteroalkyl, heteroaryl, aryl, —C₁-C₆alkylene-NH(C₁-C₆ alkyl), —C₁-C₆alkylene-N(C₁-C₆ alkyl)₂, C₃-C₆ cycloalkyl or C₂-C₅ heterocycloalkyl, wherein each of the alkyl, heteroalkyl, heteroaryl, aryl, alkylene, cycloalkyl, or heterocycloalkyl groups are optionally substituted.

Embodiment 152: The KRAS protein of embodiment 151, wherein ring Q is a C₁-C₆ optionally substituted monocyclic heterocycloalkyl.

Embodiment 153: The KRAS protein of embodiment 151, wherein ring Q is a C₅-C₉ optionally substituted bicyclic heterocycloalkyl.

Embodiment 154: The KRAS protein of embodiment 153, wherein ring Q is a Spiro bicyclic heterocycloalkyl.

Embodiment 155: The KRAS protein of any one of embodiments 151 to 154, wherein ring Q is optionally substituted with one or more R^{Q} groups, wherein each R^{Q} is independently D, halogen, —CN, —NH₂, —NH(alkyl), —N(alkyl)₂, —OH, oxo, —CO₂H, —CO₂alkyl, —C(═O)NH₂, —C(═O)NH(alkyl), —C(O)N(alkyl)₂, —S(═O)—NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

Embodiment 156: The KRAS protein of embodiment 155, wherein each R^{Q} is independently D, oxo, halogen, —CN, —NH₂, —OH, —NH(C₁-C₆ alkyl), —N(C₁-C₃alkyl)₂, —NH(cyclopropyl), C₁-C₆ alkyl, or C₁-C₆alkoxyl, wherein the alkyl or alkoxyl is optionally substituted with —CN and/or one or more halogens.

Embodiment 157: The KRAS protein of embodiment 147 or 148, wherein the electrophilic functional group comprises a structure of Formula (Ib):

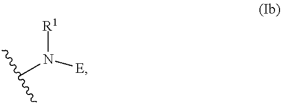

wherein,
R¹ is H, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; and
E has a structure of Formula (Ic),

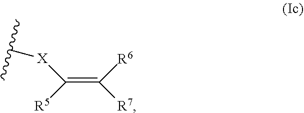

wherein,
X is C(═O), OC(═O), P(═O)OR², C(═S), S(═O)ₙ, or OS(O)ₙ;
n is 1 or 2;
R² is H or substituted or unsubstituted C₁-C₃ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; or R⁵ and R⁷ taken together form a bond;
R⁶ is H, halogen, —CN, C₁-C₆ alkyl, C₁-C₆ heteroalkyl, heteroaryl, aryl, —C₁-C₆alkylene-NH(C₁-C₆ alkyl), —C₁-C₆alkylene-N(C₁-C₆ alkyl)₂, C₃-C₆ cycloalkyl or C₂-C₅ heterocycloalkyl, wherein each of the alkyl, heteroalkyl, heteroaryl, aryl, alkylene, cycloalkyl, and heterocycloalkyl groups are optionally substituted.

Embodiment 158: The KRAS protein of any one of embodiments 151 to 157, wherein X is C(═O), P(═O)OR², S(═O), or S(O)₂.

Embodiment 159: The KRAS protein of embodiment 158, wherein X is C(═O).

Embodiment 160: The KRAS protein of any one of embodiments 157 to 159, wherein R¹ is H or substituted or unsubstituted C₁-C₆ alkyl.

Embodiment 161: The KRAS protein of embodiment 147 or 148, wherein the electrophilic functional group comprises a structure of Formula (Id),

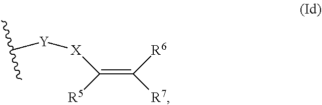

wherein,
X is C(═O), —OC(═O)—, —NR²C(═O)—, —NR²C(═NR²)—, —C(═NR)—, —NR²P(═O)OR²—, —P(═O)OR²—, C(═S), S(═O)ₙ, —OS(O)ₙ—, —NR²S(═O)ₙ—, wherein n is 1 or 2;
Y is a bond, C₁-C₆ alkylene, C₁-C₆ heteroalkylene, cycloalkyl, heterocycloalkyl, arylene or heteroarylene, each of which is optionally substituted;
each R² is independently H or substituted or unsubstituted C₁-C₃ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; or R⁵ and R⁷ taken together form a bond;
R⁶ is H, halogen, —CN, C₁-C₆ alkyl, C₁-C₆heteroalkyl, heteroaryl, aryl, —C₁-C₆alkylene-NH(C₁-C₆ alkyl), —C₁-C₆alkylene-N(C₁-C₆ alkyl)₂, C₃-C₆, cycloalkyl or C₂-C₅ heterocycloalkyl, wherein each of the alkyl, heteroalkyl, heteroaryl, aryl, cycloalkyl, and heterocycloalkyl are optionally substituted.

Embodiment 162: The KRAS protein of embodiment 161, wherein Y is substituted or unsubstituted C₁-C₄ alkylene, or substituted or unsubstituted C₁-C₄ heteroalkylene.

Embodiment 163: The KRAS protein of embodiment 161, wherein Y is substituted or unsubstituted monocyclic arylene, or substituted or unsubstituted monocyclic heteroarylene.

Embodiment 164: The KRAS protein of embodiment 161, wherein Y is substituted or unsubstituted 3 to 10 membered cycloalkyl, or substituted or unsubstituted 3 to 10 membered heterocycloalkyl.

Embodiment 165: The KRAS protein of embodiment 161, wherein Y is substituted or unsubstituted monocyclic or bicyclic heterocycloalkyl.

Embodiment 166: The KRAS protein of any one of embodiments 163 to 165, wherein Y is optionally substituted with one or more R^{Q} groups, wherein each R^{Q} is independently D, halogen. —CN, —NH₂, —NH(alkyl), —N(alkyl)₂, —OH, oxo, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

Embodiment 167: The KRAS protein of embodiment 166, wherein each R^{Q} is independently D, oxo, halogen, —CN, —NH₂, —OH, —NH(C₁-C₃alkyl), —N(C₁-C₆alkyl)₂, —NH(cyclopropyl), C₁-C₆ alkyl, or C₁-C₆ alkoxyl, wherein the alkyl or alkoxyl is optionally substituted with one or more halogens.

Embodiment 168: The KRAS protein of any one of embodiments 151 to 167, wherein R⁵ is H, halogen, methyl, or —OMe.

Embodiment 169: The KRAS protein of embodiment 168, wherein R⁵ is H.

Embodiment 170: The KRAS protein of any one of embodiments 151 to 169, wherein R⁶ is H.

Embodiment 171: The KRAS protein of any one of embodiments 151 to 170, wherein R⁷ is H or substituted or unsubstituted C₁-C₄ alkyl.

Embodiment 172: The KRAS protein of embodiment 171, wherein R⁷ is H.

Embodiment 173: The KRAS protein of any one of embodiments 151 to 167 or 170, wherein R⁵ and R⁷ taken together form a bond.

Embodiment 174: The KRAS protein of any one of embodiments 151 to 164), wherein E is

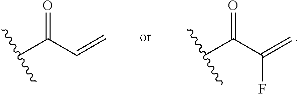

Embodiment 175: The KRAS protein of any one of embodiments 161 to 167, wherein

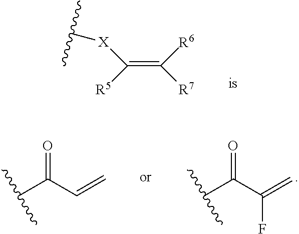

Embodiment 176: The KRAS protein of embodiment 147 or 148, wherein the electrophilic functional group comprises a
structure selected from

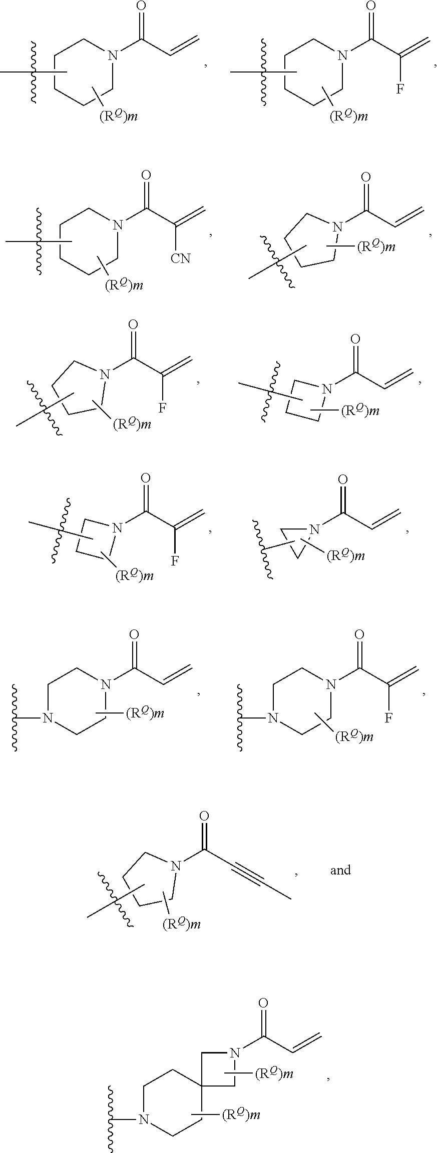

wherein
each R^{Q} is independently D, oxo, halogen, —CN, —NH₂, —OH, —NH(C₁-C₃alkyl), —N(C₁-C₃alkyl)₂. —NH(cyclopropyl), C₁-C₆alkyl, or C₁-₆alkoxyl, wherein the alkyl or alkoxyl is optionally substituted with one or more halogens, and m is 0. 1, 2.3, 4, or 5.

Embodiment 177: The KRAS protein of any one of embodiments 155, 166, or 176, wherein each R^{Q} is independently substituted or unsubstituted C₁-C₃ alkyl, amino, or —CN.

Embodiment 178: The KRAS protein of embodiment 177, wherein each R^{Q} is methyl, —CH₂CN, or CN.

Embodiment 179: The KRAS protein of embodiment 147 or 148, wherein the electrophilic functional group comprises a
structure selected from

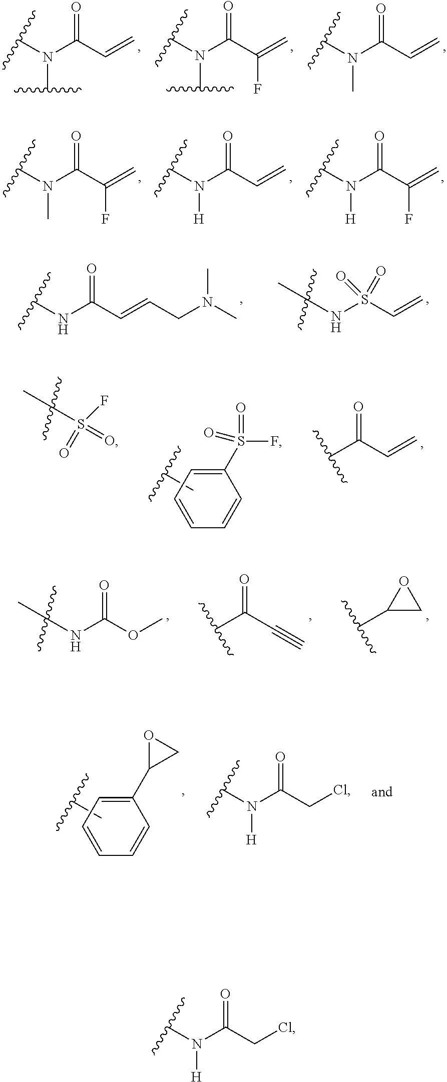

wherein

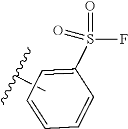

and

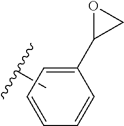

optionally substituted.

Embodiment 180: The KRAS protein of any one of embodiments 143 to 179, wherein the radiolabeled compound comprises a linker connecting the radioisotope.

Embodiment 181: The KRAS protein of embodiment 180, wherein the linker comprises C₁-C₁₀ alkylene or C₁-C₁₀ heteroalkylene, wherein the alkylene or heteroalkylene is optionally substituted with one or more substituents selected from halogen, amino, —OH, —NO₂, oxo, —CN, C₁₋₃alkoxyl, C₁₋₃alkyl, C₁₋₃hydroxyalkyl, C₁₋₃aminoalkyl, and C₁₋₃haloalkyl.

Embodiment 182: The KRAS protein of embodiment 181, wherein the linker comprises C₁-C₆ alkylene.

Embodiment 183: The KRAS protein of any one of embodiments 180 to 182, wherein the linker comprises one or more functional groups selected from: —O—, —S—, —S—S—, —C(═O)O—, —OC(═O)—, —C(═O)NR^{a}—, —NR^{a}C(═O)—, —S(═O)₂NR^{a}—, —NR^{a}S(═O)₂—, —NR^{a}C(═O)NR^{a}—, —NR^{a}C(═O)O—, and —OC(═O)NR^{a}—,
wherein each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl.

Embodiment 184: The KRAS protein of any one of embodiments 180 to 182, wherein the linker comprises 1 to 20
groups independently selected from —CR^{b}R^{b}—, —C(═O)—, —S(═O)—, —S(═O)—, —NR^{a}—,

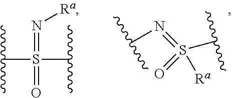

CR^{b}═CR^{b}—, —C≡C—, —O—, —S—, —C(═O)O—, —OC(═O)—, —C(═O)NR^{a}—, —NR^{a}C(═O)—, —S(═O)₂NR^{a}—, —NR^{a}S(═O)₂—, —NR^{a}C(═O)NR^{a}—, —NR^{a}C(═O)O—, —OC(═O)NR^{a}—, arylene, heteroarylene,
wherein each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₁-C₆alkynyl, C₁-C₆ cycloalkyl, C₂-C₉heterocycloalkyl, aryl, or heteroaryl, and wherein each R^{b} is independently hydrogen, halogen, —CN, —NO₂, —OR^{a}, —SR^{a}, C₁-C₆alkyl, C₂-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl.

Embodiment 185: The KRAS protein of embodiment 183 or 184, wherein the linker comprises

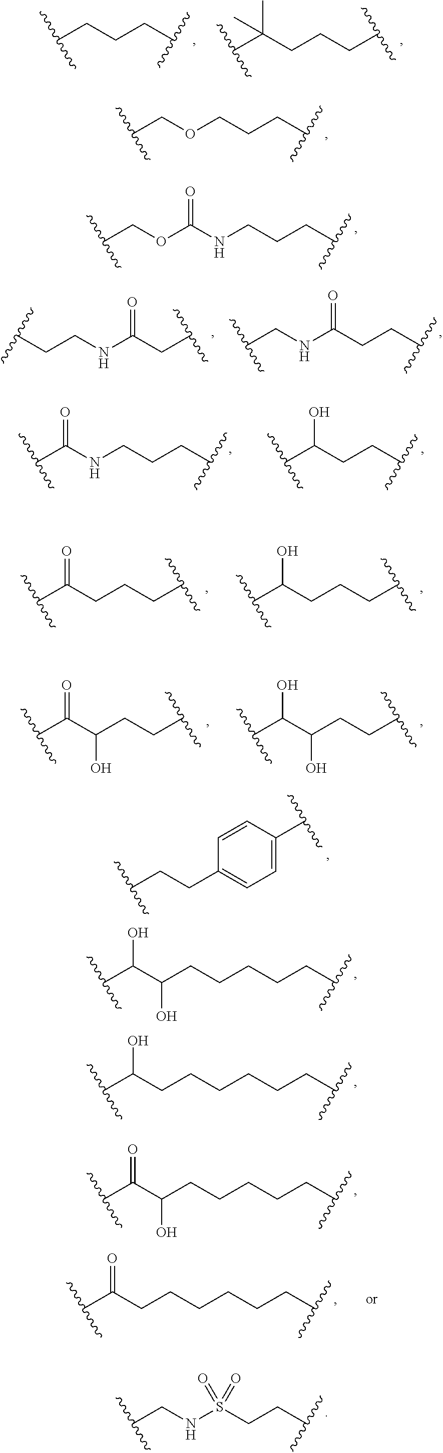

Embodiment 186: The KRAS protein of embodiment 183 or 184, wherein the linker comprises

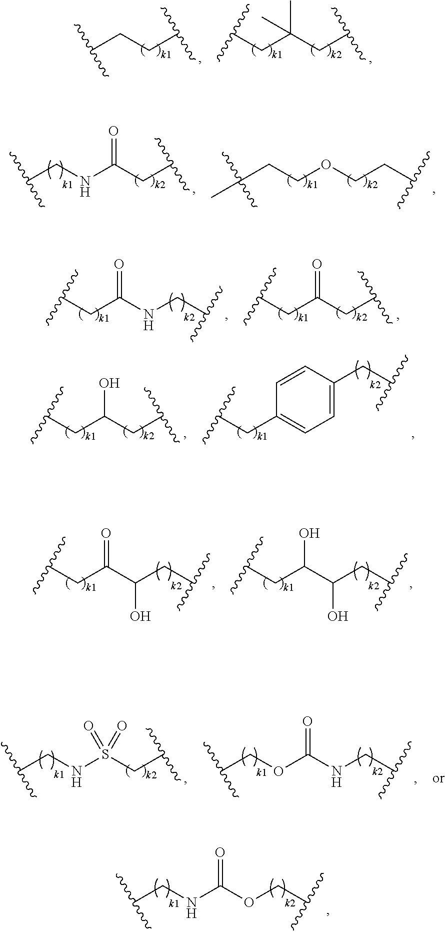

wherein each k1 and k2 is independently 0 or an integer selected from 1 to 10.

Embodiment 187: The KRAS protein of embodiment 183 or 184, wherein the linker comprises

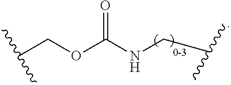

Embodiment 188: The KRAS protein of any one of embodiments 180 to 186, wherein the linker is a brush border enzyme-cleavable linker.

Embodiment 189: The KRAS protein of any one of embodiments 180 to 186, wherein the linker is a hepatocyte-cleavable linker.

Embodiment 190: The KRAS protein of any one of embodiments 180 to 186, wherein the linker is a cytochrome P450-substrate.

Embodiment 191: The KRAS protein of any one of embodiments 180 to 186, wherein the linker is an esterase-cleavable linker.

Embodiment 192: The KRAS protein of any one of embodiments 180 to 186, wherein the linker is a peptidase-cleavable linker.

Embodiment 193: The KRAS protein of any one of embodiments 143 to 192, wherein the radiolabeled compound comprises a structure of Formula (Va), Formula (Vb), Formula (Vc), Formula (Vd), or Formula (Ve):

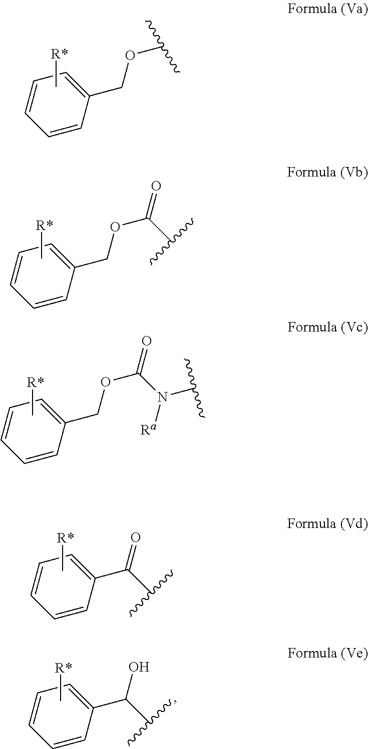

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At); and
R^{a} is hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₄heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl, or heteroaryl is optionally substituted.

Embodiment 194: The KRAS protein of embodiment 193, wherein the alkyl, haloalkyl, hydroxyalkyl, aminoalkyl, heteroalkyl, alkenyl, alkynyl, cycloalkyl, heterocycloalkyl, aryl, or heteroaryl are independently optionally substituted by one or more halogen, amino, —OH, —NO₂, oxo, —CN, C₁₋₃alkoxyl, C₁₋₃alkyl and C₁₋₃haloalkyl.

Embodiment 195: The KRAS protein of embodiment 193, wherein the radiolabeled compound comprises a structure of

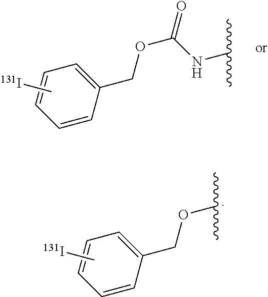

Embodiment 196: The KRAS protein of any one of embodiments 143 to 148, wherein the radiolabeled compound comprises a structure of Formula (III), or a salt or solvate thereof,

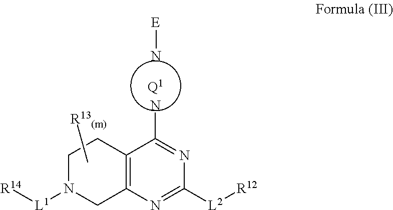

wherein
ring Q¹ is a 4-12 membered saturated or partially saturated monocyclic or bicyclic ring, wherein the monocyclic or bicyclic ring is optionally substituted;
L¹ is a bond, —C(═O)—, O, S, NR¹⁵, optionally substituted C₁-C₃ alkylene, optionally substituted C₁-C₃ heteroalkylene;
L² is a bond, —C(═O)—, O, S or NR¹⁵;
E is

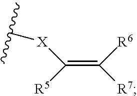

X is C(═O), P(═O)OR², C(═S), or S(═O), where n is 1 or 2;
R² is H or optionally substituted C₁-C₃ alkyl;
R⁵ is hydrogen, cyano, halogen, C₁-C₆ alkyl, C₁-C₆ alkoxyl, C₁-C₆heteroalkyl, C₃-C₆ cycloalkyl, or C₂-C₄ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₁-C₃ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R¹² is hydrogen, C₁-C₆ alkyl, C₁-C₆ heteroalkyl, -L³—NR¹⁵R^{15′}, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted; each R¹³ is independently OH, halogen, oxo, substituted or unsubstituted C₁-C₆ alkyl, or substituted or unsubstituted C₁-C₆ heteroalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or optionally substituted C₁-C₃ alkyl;
each R^{15′} is independently hydrogen, acyl, optionally substituted C₁-C₃ alkyl, optionally substituted C₁-C₃ heteroalkyl or optionally substituted C₁-C₃ hydroxyalkyl; and
m is 0, 1, or 2.

Embodiment 197: The KRAS protein of embodiment 196, wherein
ring Q¹ is a 4-12 membered saturated or partially saturated monocyclic or bicyclic ring optionally substituted with one, two, or three groups selected from R¹⁸;
L¹ is a bond, —C(═O)—, O. S. NR¹⁵, optionally substituted C₁-C₃ alkylene, or optionally substituted C₁-C₆ heteroalkylene, wherein the alkylene and heteroalkylene is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅ heterocycloalkyl.
L² is a bond, —C(═O)—, O, S or NR¹⁵;
E is

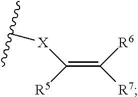

X is C(═O), P(═O)OR², C(═S), or S(═O)ₙ, where n is 1 or 2;
R² is H or substituted or unsubstituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₆ heterocycloalkyl.
R⁵ is hydrogen, cyano, halogen, C₁-C₆ alkyl, C₁-C₆ alkoxyl, C₁-C₆ heteroalkyl, C₃-C₆ cycloalkyl, or C₂-C₅ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₁-C₆cycloalkyl, and C₂-C₅ heterocycloalkyl.
R⁷ is hydrogen, cyano, halogen, C₁-C₆ alkyl, C₁-C₆ alkoxyl, C₁-C₆ heteroalkyl, C₁-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅heterocycloalkyl; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyano, halogen, C₁-C₆ alkyl, C₁-C₆ alkoxyl, C₁-C₆ heteroalkyl, C₁-C₆ cycloalkyl, C₂-C₆ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted with one, two, or three groups selected from halogen, —CN, hydroxy, C₁-C₆, alkyl, C₁-C₆ haloalkyl, C₁-C₆ alkoxy, C₃-C₆ cycloalkyl, and C₂-C₅ heterocycloalkyl;
R¹² is hydrogen, C₁-C₆ alkyl, C₁-C₆ heteroalkyl, -L³—NR¹⁵R^{15′}, heterocycloalkyl, -L³-heterocycloalkyl, cycloalkyl, -L³-cycloalkyl, aryl, heteroaryl, -L³-aryl, or -L-heteroaryl, wherein each of the heterocycloalkyl, cycloalkyl, aryl, heteroaryl, alkyl or heteroalkyl is optionally substituted with one, two, three or four groups selected from R¹⁹;
L₃ is C₁-C₄ alkylene or C₁-C₄ heteroalkylene, each of which is optionally substituted with one, two, or three groups selected from R¹⁹;
each R¹³ is independently OH, halogen, oxo, substituted or unsubstituted C₁-C₆ alkyl, or substituted or unsubstituted C₁-C₆ heteroalkyl, wherein each of the alkyl and heteroalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₁-C₅heterocycloalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted with one, two, three or four groups selected from R¹⁶;
each R¹⁵ is independently hydrogen or optionally substituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₈ heterocycloalkyl;
each R¹⁵ is independently hydrogen, acyl, optionally substituted C₁-C₃ alkyl, optionally substituted C₁-C₃ heteroalkyl or optionally substituted C₁-C₃ hydroxyalkyl;
m is 0, 1, or 2;
each R¹⁷ is independently halogen, hydroxyl, C₁-C₆ alkyl, C₁-C₆ cycloalkyl, C₁-C₆ alkoxy, C₁-C₆ haloalkyl, amino, cyano, C₁-C₆, heteroalkyl, C₁-C₆ hydroxyalkyl, —O—C₁-C₆; haloalkyl, or —S—C₁-C₆haloalkyl.
each R¹⁸ is independently halogen, oxo, C₁-C₆, alkyl, C₁-C₆ aminoalkyl, C₁-C₆ haloalkyl, C₂-C₆ alkenyl, C₂-C₆ alkynyl, C₁-C₆ heteroalkyl, —CN, —C(O)OR¹⁵, —C(O)N(R¹⁵)(R^{15′}), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, and C₂-C₅ heterocycloalkyl;
R¹⁶ and R¹⁹ are each independently selected from halogen, oxo, —CN, C₁-C₆ alkyl, C₂-C₆ alkenyl, C₂-C₆ alkynyl, C₁-C₆ heteroalkyl, C₃-C₁₀cycloalkyl, —C₁₋₃alkylene-C₃₋₆cycloalkyl, C₂-C₉ heterocycloalkyl, —C₁₋₃alkylene-C₂₋₉heterocycloalkyl, C₆-C₁₀aryl, —C₁₋₃alkylene-C₃₋₆aryl, C₁-C₉heteroaryl, —C₁₋₃alkylene-C₂₋₉heteroaryl, —OR¹⁰, —SR¹⁰. —N(R¹⁰)(R^{10′}), —C(O)OR¹⁰, —OC(═O)N(R¹⁰)(R^{10′}), —N(R^{10′})C(O)N(R¹⁰)(R^{10′}), —N(R^{10′})C(O)OR¹⁰, —N(R^{10′})S(O)₂R¹¹, —C(O)R¹¹, —S(O)R¹¹, —OC(═O)R¹¹, —C(O)N(R¹⁰)(R^{10′}), —C(O)C(O)N(R¹⁰)(R^{10′}), —N(R^{10′})C(O)R¹¹, —S(O)₂R¹¹, —S(O)₂N(R¹⁰)(R^{10′})—, S(═O)(═NH)N(R¹⁰)(R^{10′}), —CH₂C(O)N(R¹⁰)(R^{10′}), —CH₂N(R^{10′})C(O)R¹¹, —CH₂S(O)₂R¹¹, and —CH—S(O)₂N(R¹⁰)(R^{10′}), wherein the alkyl, alkenyl, alkynyl, cycloalkyl, —C₁₋₃alkylene-C₆₋₁₀cycloalkyl, heterocycloalkyl, —C₁₋₃alkylene-C₂-C₆heterocycloalkyl, aryl, —C₁₋₃alkylene-C₆₋₁₀aryl, heteroaryl and —C₁₋₃alkylene-C₂₋₉heteroaryl are optionally substituted with one, two, three, or four groups independently selected from halogen, oxo, —CN, C₁-C₆ alkyl, C₁-C₆ haloalkyl, C₁-C₆ alkoxy, C₁-C₆ alkyloxy, C₁-C₆ haloalkoxy, C₃-C₁₀cycloalkyl, C₂-C₉ heterocycloalkyl, C₆-C₁₀ aryl, C₁-C₉ heteroaryl, —OR¹⁰, —SR¹⁰, —N(R¹⁰)(R^{10′}), —C(O)OR¹⁰, —OC(═O)N(R¹⁰)(R^{10′}), —N(R^{10′})C(O)N(R¹⁰)(R^{10′}). —N(R¹⁰)C(O)OR¹⁵, —N(R^{10′})S(O)₂R¹¹, —C(O)R¹¹, —S(O)R¹¹, —OC(═O)R¹¹, —C(O)N(R¹⁰)(R^{10′}), —C(O)C(O)N(R¹⁰)(R^{10′}), —N(R^{10′})C(O)R¹¹, —S(O)₂R¹¹, —S(O)₂N(R¹⁰)(R^{10′})—, S(═O)(═NH)N(R¹⁰)(R^{10′}), —CH₂C(O)N(R¹⁰)(R^{10′}), —CH₂N(R^{10′})C(O)R¹¹, —CH₂S(O)₂R¹¹, and —CH₂S(O)₂N(R¹⁰)(R^{10′});
each R¹⁰ is independently selected from hydrogen, C₁-C₆, alkyl, C₁-C₆ haloalkyl, C₂-C₆ alkenyl, C₂-C₆ alkynyl, C₃-C₆ cycloalkyl, C₂-C₉heterocycloalkyl, C₆-C₁₀ aryl, and C₁-C₆heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₆ heteroaryl,
each R^{10′} is independently selected from hydrogen, C₁-C₆ alkyl, and C₁-C₆ haloalkyl; and
each R¹¹ is independently selected C₁-C₆ alkyl, C₂-C₆ alkenyl, C₂-C₆ alkynyl, C₃-C₆cycloalkyl, C₂-C₉ heterocycloalkyl, C₆-C₁₀ aryl, and C₁-C₉ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₂-C₅ alkyl, C₁-C₆ haloalkyl, C₁-C₆, alkoxy, C₃-C₆ cycloalkyl, C₂-C₇ heterocycloalkyl, C₆-C₁₀ aryl, and C₁-C₉heteroaryl.

Embodiment 198: The KRAS protein of embodiment 196 or 197, wherein at least one of L¹, L². R¹², R¹³ and R¹⁴ comprises the covalently bonded radioisotope.

Embodiment 199: The KRAS protein of any one of embodiments 196 to 198, wherein the covalently bonded radioisotope is R* and R* is fluorine-18 (¹⁸F), iodine-131 (¹³¹I), iodine-123 (¹²³I), iodine-124 (¹²⁴I), iodine-125 (¹²⁵I), or astatine-211 (²¹¹At).

Embodiment 200: The KRAS protein of any one of embodiments 196 to 198, wherein the covalently bonded radioisotope is R* and R* is iodine-131.

Embodiment 201: The KRAS protein of any one of embodiments 196 to 200, wherein R⁵ is hydrogen, cyano, halogen, optionally substituted C₁-C₆ alkyl, optionally substituted C₁-C₆ alkoxyl, optionally substituted C₁-C₆ heteroalkyl, optionally substituted C₃-C₆ cycloalkyl, or optionally substituted C₂-C₅ heterocycloalkyl.

Embodiment 202: The KRAS protein of embodiment 201, wherein R⁵ is hydrogen, halogen or a C₁-C₃ alkyl optionally substituted with one to three substituents selected from hydroxyl and halogen.

Embodiment 203: The KRAS protein of embodiment 202, wherein R⁵ is a hydrogen.

Embodiment 204: The KRAS protein of embodiment 202, wherein R⁵ is a halogen.

Embodiment 205: The KRAS protein of embodiment 202, wherein R⁵ is a fluorine.

Embodiment 206: The KRAS protein of any one of embodiments 196 to 205, wherein R⁷ is hydrogen, cyano, halogen, optionally substituted C₁-C₆ alkyl, optionally substituted C₂-C₅ alkoxyl, or optionally substituted C₁-C₆ heteroalkyl.

Embodiment 207: The KRAS protein of any one of embodiments 196 to 205, wherein R⁷ is H.

Embodiment 208: The KRAS protein of any one of embodiments 196 to 200, wherein R⁵ and R⁷ taken together with the carbon atoms to which they are attached form a 5-8 membered partially saturated cycloalkyl, wherein the cycloalkyl is optionally substituted.

Embodiment 209: The KRAS protein of any one of embodiments 196 to 208, wherein R⁶ is hydrogen, cyano, halogen, optionally substituted C₁-C₆ alkyl, optionally substituted C₂-C₅ alkoxyl, optionally substituted C₁-C₆heteroalkyl, optionally substituted C₃-C₆ cycloalkyl, or optionally substituted C₂-C₆ heterocycloalkyl.

Embodiment 210: The KRAS protein of any one of embodiments 196 to 208, wherein R⁶ is hydrogen.

Embodiment 211: The KRAS protein of any one of embodiments 196 to 210, wherein ring Q¹ is optionally substituted with one to three R,
wherein R¹⁸ is oxo, C₁-C₆ alkyl, C₂-C₆ alkenyl, C₂-C₆ alkynyl, C₁-C₆ heteroalkyl, cyano, —C(O)OR¹⁵, —C(O)N(R¹⁵)(R^{15′}), or —N(R¹⁵)(R^{15′}), wherein the alkyl, alkenyl, alkynyl, or heteroalkyl is optionally substituted.

Embodiment 212: The KRAS protein of embodiment 211, wherein ring Q¹ is a 6 membered monocyclic ring optionally substituted with one to three R¹⁸, wherein R¹⁸ is methyl. —CH₂CN, oxo, hydroxyl, carboxyl, C(O)OR¹⁵.

Embodiment 213: The KRAS protein of any one of embodiments 196 to 212, wherein the radiolabeled compound comprises a structure of Formula (IIIa), or a salt or solvate thereof,

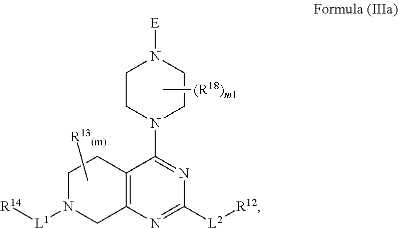

wherein m1 is 0, 1, 2, or 3.

Embodiment 214: The KRAS protein of any one of embodiments 196 to 213, wherein R¹⁴ is hydrogen, cycloalkyl, heterocycloalkyl, aryl, or heteroaryl, wherein each of the cycloalkyl, heterocycloalkyl, aryl, and heteroaryl is optionally substituted with one or more R¹⁶ wherein each R¹⁶ is independently halogen, hydroxyl, C₁-C₆ alkyl, cycloalkyl, C₁-C₆ alkoxy, acetyl, oxo, —C(O)OR¹⁵, C₁-C₆ haloalkyl, amino, cyano, C₁-C₆ heteroalkyl, C₁-C₆ hydroxyalkyl, —O— C₁-C₆haloalkyl, or —S— C₁-C₆haloalkyl.

Embodiment 215: The KRAS protein of any one of embodiments 196 to 214, wherein R¹⁴ is phenyl, napthyl, or monocyclic or bicyclic heteroaryl, each optionally substituted with one or more R¹⁶, wherein each R¹⁶ is independently halogen, —CN, —NH₂, —NH(C₁-C₆alkyl), —N(C₁-C₆alkyl)₂, —OH, oxo, —CO₂H, —C(═O)O—C₁₋₆alkyl, —C(O)NH₂, —C(═O)NH(C₁-C₆alkyl), —C(═O)N(C₁-C₆alkyl)₂, —S(═O)₂NH₂, —S(═O)₂NH(C₁-C₆alkyl), —S(═O)₂N(C₁-C₆alkyl)₂, C₁-C₆ alkyl, C₃-C₆ cycloalkyl, C₁-C₆fluoroalkyl, C₁-C₆heteroalkyl, C₁-C₆alkoxy, C₁-C₆fluoroalkoxy, C₁-C₆ heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

Embodiment 216: The KRAS protein of any one of embodiments 196 to 215, wherein each R¹⁶ is independently oxo, halogen, —CN, —NH₂, —NH(CH₃), —N(CH₃)₂, —OH, —CO₂H, —CO₂(C₁-C₄ alkyd), —OCO(C₁-C₄alkyl), —C(═O)NH₂, —C(═O)NH(C₁-C₄ alkyl), —C(═O)N(C₁-C₄alkyl)₂, —S(═O), NH₂, —S(═O)₂NH(C₁-C₄ alkyl), —S(═O)₂N(C₁-C₄alkyl)₂, C₁-C₄ alkyl, C₃-C₆cycloalkyl, C₁-C₄ fluoroalkyl, C₁-C₄ heteroalkyl, C₁-C₄ alkoxy, C₁-C₄ fluoroalkoxy, —S—C₁₋₄ alkyl, —S(O)C₁₋₄ alkyl, or —S(═O)₂(C₁-C₄ alkyl).

Embodiment 217: The KRAS protein of any one of embodiments 196 to 216, wherein R¹⁴ is napthyl optionally substituted with one or more R¹⁶, wherein each R¹⁶ is independently halogen, hydroxyl, C₁-C₃ alkyl, alkoxy, haloalkyl, amino, or cyano.

Embodiment 218: The KRAS protein of any one of embodiments 196 to 217, wherein R¹⁴ is halogen and the halogen is a radioisotope selected from fluorine-18 (¹⁸F), iodine-131 (¹³¹I), iodine-123 (¹²³I), iodine-124 (¹²⁴I), iodine-125 (¹²⁵I), and astatine-211 (²¹¹At).

Embodiment 219: The KRAS protein of any one of embodiments 196 to 217, wherein R¹⁴ comprises the covalently bonded radioisotope R*.

Embodiment 220: The KRAS protein of any of embodiments 54 to 57, wherein R¹⁴ is

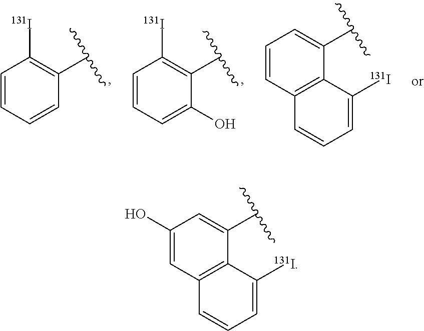

Embodiment 221: The KRAS protein of any one of embodiments 196 to 220,
wherein R¹² is hydrogen, C₁-C₆ alkyl, C₁-C₆ heteroalkyl, -L³—NR¹⁵R^{15′}, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted with one or more selected from R¹⁹;
wherein each R¹⁹ is independently oxo, acyl, hydroxyl, cyano, amino, halogen, C₁-C₆ alkyl, C₁-C₆, hydroxyalkyl, C₁-C₆ heteroalkyl, C₃-C₁₀ cycloalkyl, C₂-C₉ heterocycloalkyl, or C₁-C₆ alkoxy, wherein the alkyl, hydroxyalkyl, alkoxy, heteroalkyl, cycloalkyl, or heterocycloalkyl is optionally substituted.

Embodiment 222: The KRAS protein of embodiment 221, wherein R¹² is cycloalkyl, heterocycloalkyl, -L³-heterocycloalkyl, or -L³-cycloalkyl, wherein each of the L³, heterocycloalkyl, cycloalkyl, alkyl, or heteroalkyl is optionally substituted with one or more R¹⁹.

Embodiment 223: The KRAS protein of embodiment 221 or 222, wherein L³ is C₁-C₄ alkylene or C₁-C₄ heteroalkylene, each of which is optionally substituted with one or more R¹⁹.

Embodiment 224: The KRAS protein of any one of embodiments 221 to 223, wherein each R¹⁹ is independently oxo, acyl, hydroxyl, cyano, halogen, C₁-C₆ alkyl, or C₁-C₆ hydroxyalkyl containing 1 to 3 hydroxyl groups.

Embodiment 225: The KRAS protein of any one of embodiments 196 to 224, wherein X is C(═O).

Embodiment 226: The KRAS protein of any one of embodiments 196 to 225, wherein L¹ is a bond.

Embodiment 227: The KRAS protein of any one of embodiments 196 to 226, wherein L² is a bond, O, S or NR¹⁵.

Embodiment 228: The KRAS protein of any one of embodiments 196 to 227, wherein each R¹³ is independently OH, halogen, or C₁-C₃ alkyl.

Embodiment 229: The KRAS protein of any one of embodiments 196 to 228, wherein m is 0 or 1.

Embodiment 230: The KRAS protein of any one of embodiments 196 to 229, wherein the radiolabeled compound has a structure of

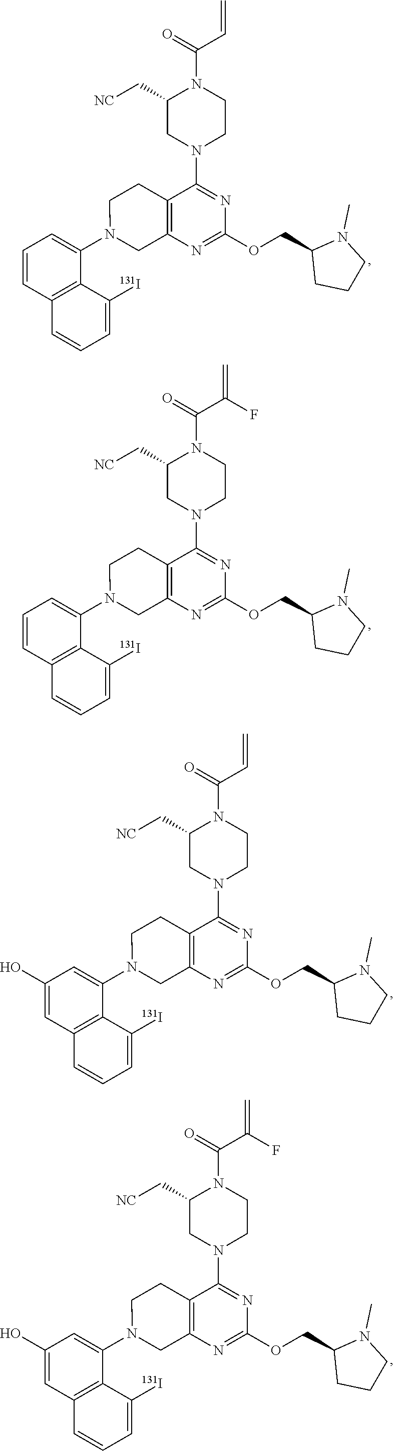 [SMILES: C=CC(=O)N1CCN(c2nc(OC[C@@H]3CCCN3C)nc3c2CCN(c2cccc4cccc([131I])c24)C3)C[C@@H]1CC#N ; C=C(F)C(=O)N1CCN(c2nc(OC[C@@H]3CCCN3C)nc3c2CCN(c2cccc4cccc([131I])c24)C3)C[C@@H]1CC#N ; C=CC(=O)N1CCN(c2nc(OC[C@@H]3CCCN3C)nc3c2CCN(c2cc(O)cc4cccc([131I])c24)C3)C[C@@H]1CC#N ; C=C(F)C(=O)N1CCN(c2nc(OC[C@@H]3CCCN3C)nc3c2CCN(c2cc(O)cc4cccc([131I])c24)C3)C[C@@H]1CC#N]

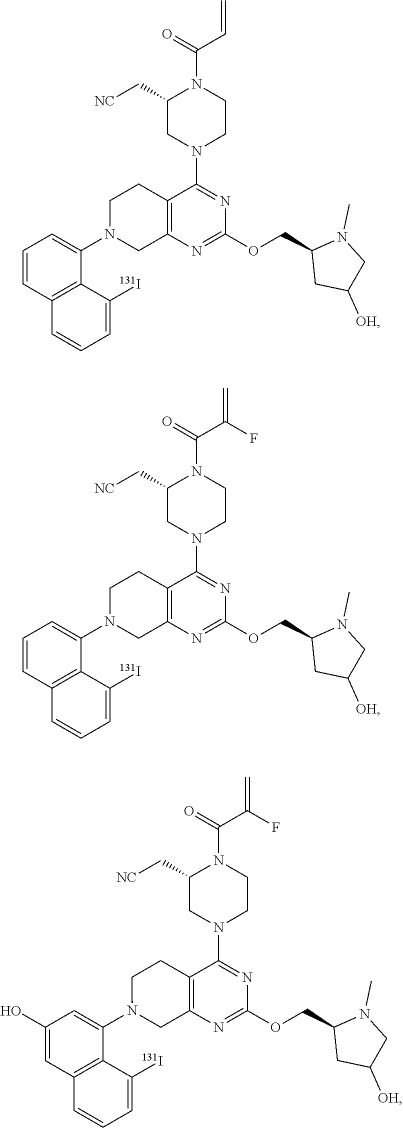 [SMILES: C=CC(=O)N1CCN(c2nc(OC[C@@H]3CC(O)CN3C)nc3c2CCN(c2cccc4cccc([131I])c24)C3)C[C@@H]1CC#N ; C=C(F)C(=O)N1CCN(c2nc(OC[C@@H]3CC(O)CN3C)nc3c2CCN(c2cccc4cccc([131I])c24)C3)C[C@@H]1CC#N ; C=C(F)C(=O)N1CCN(c2nc(OC[C@@H]3CC(O)CN3C)nc3c2CCN(c2cc(O)cc4cccc([131I])c24)C3)C[C@@H]1CC#N]

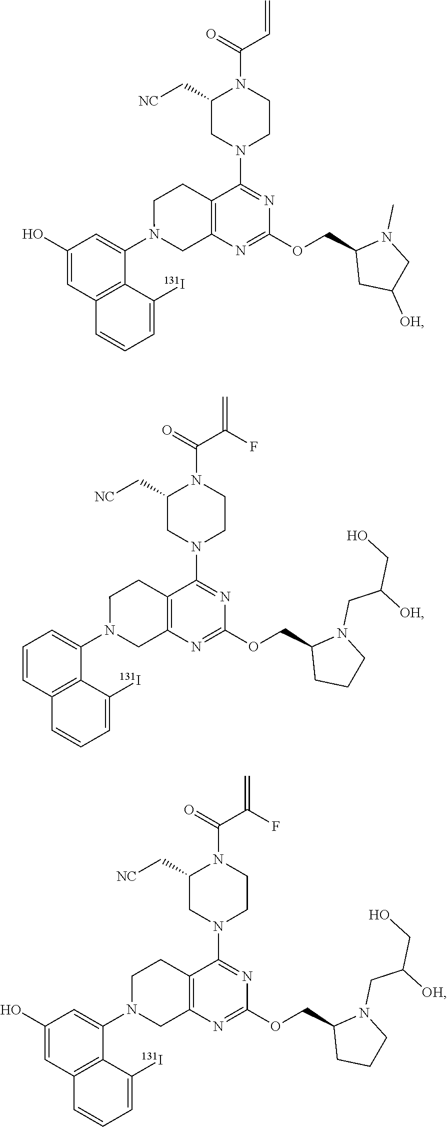 [SMILES: C=CC(=O)N1CCN(c2nc(OC[C@@H]3CC(O)CN3C)nc3c2CCN(c2cc(O)cc4cccc([131I])c24)C3)C[C@@H]1CC#N ; C=C(F)C(=O)N1CCN(c2nc(OC[C@@H]3CCCN3CC(O)CO)nc3c2CCN(c2cccc4cccc([131I])c24)C3)C[C@@H]1CC#N ; C=C(F)C(=O)N1CCN(c2nc(OC[C@@H]3CCCN3CC(O)CO)nc3c2CCN(c2cc(O)cc4cccc([131I])c24)C3)C[C@@H]1CC#N]

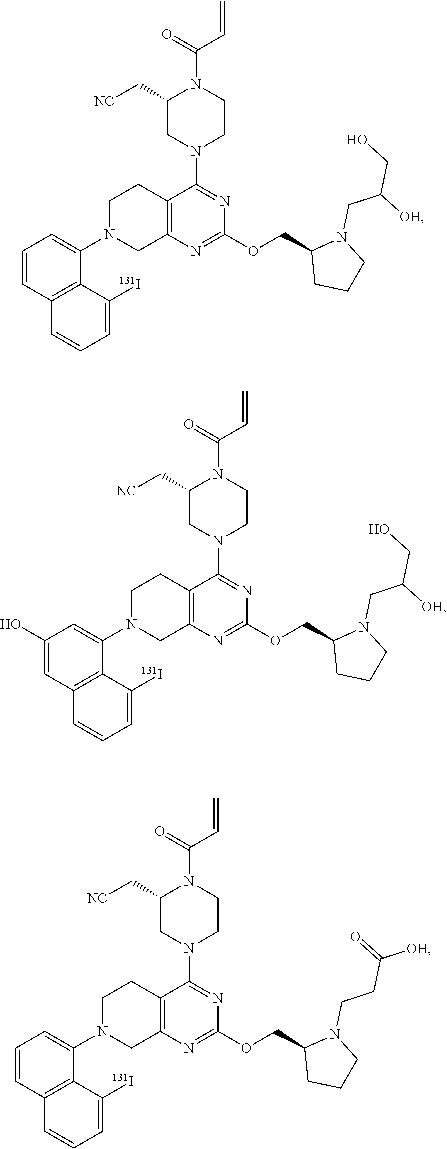 [SMILES: C=CC(=O)N1CCN(c2nc(OC[C@@H]3CCCN3CC(O)CO)nc3c2CCN(c2cccc4cccc([131I])c24)C3)C[C@@H]1CC#N ; C=CC(=O)N1CCN(c2nc(OC[C@@H]3CCCN3CC(O)CO)nc3c2CCN(c2cc(O)cc4cccc([131I])c24)C3)C[C@@H]1CC#N ; C=CC(=O)N1CCN(c2nc(OC[C@@H]3CCCN3CCC(=O)O)nc3c2CCN(c2cccc4cccc([131I])c24)C3)C[C@@H]1CC#N]

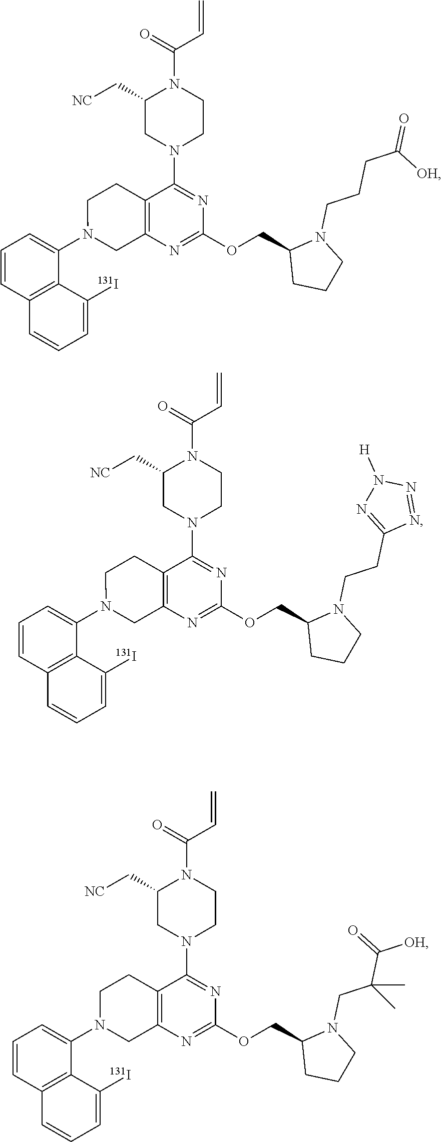 [SMILES: C=CC(=O)N1CCN(c2nc(OC[C@@H]3CCCN3CCCC(=O)O)nc3c2CCN(c2cccc4cccc([131I])c24)C3)C[C@@H]1CC#N ; C=CC(=O)N1CCN(c2nc(OC[C@@H]3CCCN3CCc3nn[nH]n3)nc3c2CCN(c2cccc4cccc([131I])c24)C3)C[C@@H]1CC#N ; C=CC(=O)N1CCN(c2nc(OC[C@@H]3CCCN3CC(C)(C)C(=O)O)nc3c2CCN(c2cccc4cccc([131I])c24)C3)C[C@@H]1CC#N]

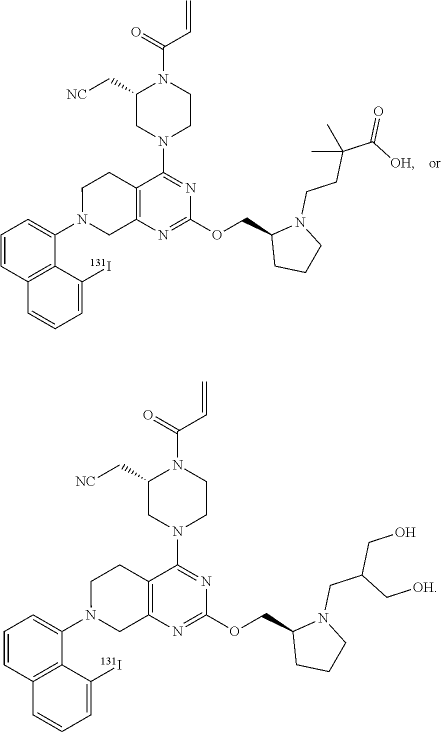 [SMILES: C=CC(=O)N1CCN(c2nc(OC[C@@H]3CCCN3CCC(C)(C)C(=O)O)nc3c2CCN(c2cccc4cccc([131I])c24)C3)C[C@@H]1CC#N ; C=CC(=O)N1CCN(c2nc(OC[C@@H]3CCCN3CC(CO)CO)nc3c2CCN(c2cccc4cccc([131I])c24)C3)C[C@@H]1CC#N]

Embodiment 231: The KRAS protein of any one of embodiments 196-217 or 221-229, wherein the radiolabeled compound comprises;
a) the structure of Formula (III) or a salt or solvate thereof;
b) the covalently bonded radioisotope; and
c) a linker covalently bonded to the radioisotope and to the structure of Formula (III) or a salt or solvate thereof.

Embodiment 232: The KRAS protein of embodiment 231, where in the linker comprises C₁-C₁₀ alkylene or C₁-C₁₀ heteroalkylene, wherein the alkylene or heteroalkylene is optionally substituted with one or more substituents selected from halogen, amino, —OH, —NO₂, oxo, —CN, C₁₋₃alkoxyl, C₁₋₃alkyl, C₁₋₃hydroxyalkyl, C₁₋₃aminoalkyl, and C₁₋₃haloalkyl.

Embodiment 233: The KRAS protein of embodiment 232, wherein the linker comprises C₁-C₆ alkylene.

Embodiment 234: The KRAS protein of any one of embodiments 231 to 233, wherein the linker comprises one or more functional groups selected from: —O—, —S—, —S—S—. —C(═O)—, —OC(═O)—, —C(═O)NR^{a}—, —NR^{a}C(═O)—, —S(═O)₂NR^{a}—, —NR^{a}S(═O)₂—, —NR^{a}C(═O)NR^{a}—, —NR^{a}C(═O)O—, and —OC(═O)NR^{a}—,
wherein each R^{a} is independently hydrogen, halogen, C₁-C₆alkyl, C₁-C₆haloalkyl,
C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl.

Embodiment 235: The KRAS protein of any one of embodiments 231 to 234, wherein the linker comprises

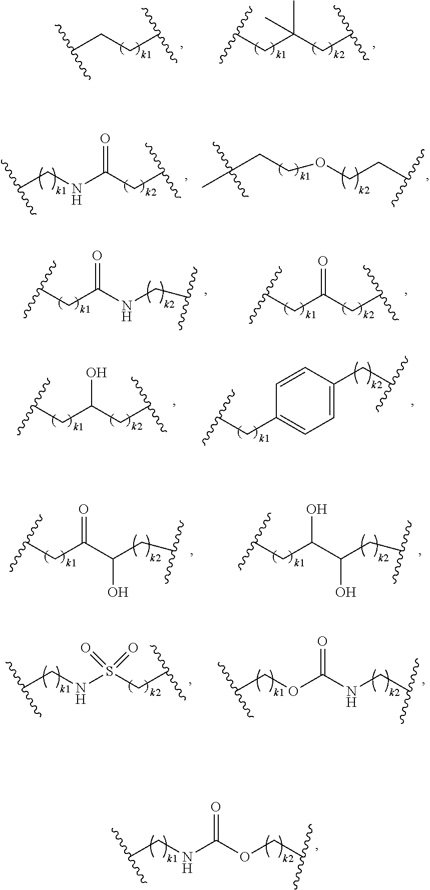

wherein each k1 and k2 is independently 0 or an integer selected from 1 to 10.

Embodiment 236: The KRAS protein of any one of embodiments 231 to 235, wherein the linker comprises

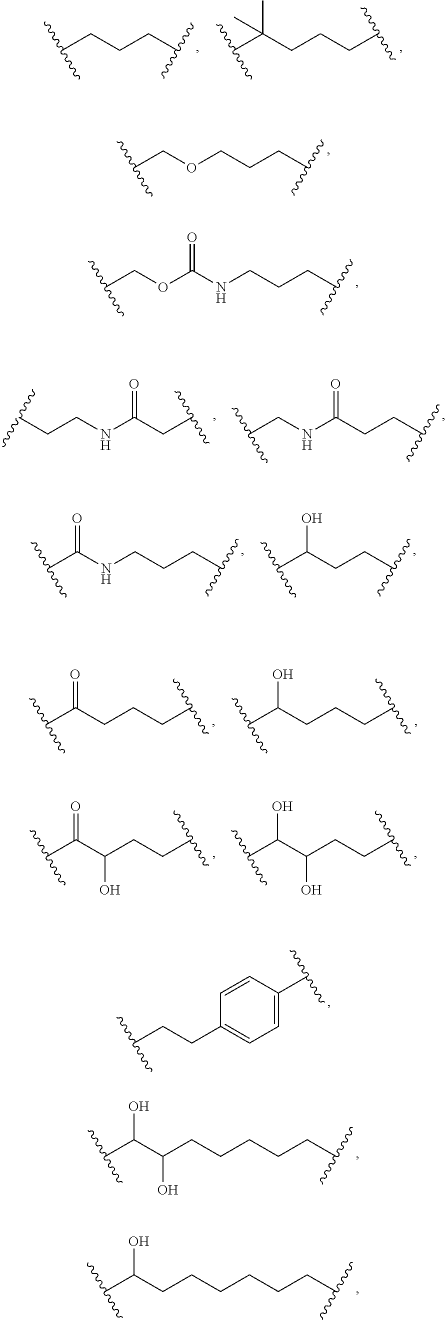

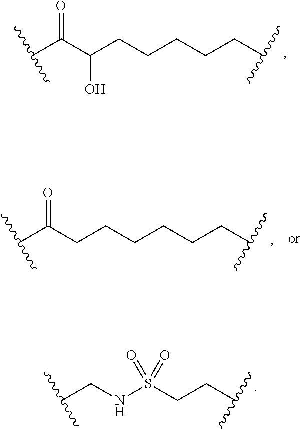 [SMILES: *CCCCCC(O)C(*)=O ; *CCCCCCC(*)=O ; *CCS(=O)(=O)NC*]

Embodiment 237: The KRAS protein of any one of embodiments 231 to 236, wherein the linker is a brush border enzyme-cleavable linker.

Embodiment 238: The KRAS protein of any one of embodiments 231 to 236, wherein the linker is a hepatocyte-cleavable linker.

Embodiment 239: The KRAS protein of any one of embodiments 231 to 236, wherein the linker is a cytochrome P450-substrate.

Embodiment 240: The KRAS protein of any one of embodiments 231 to 236, wherein the linker is an esterase-cleavable linker.

Embodiment 241: The KRAS protein of any one of embodiments 231 to 236, wherein the linker is a peptidase-cleavable linker.

Embodiment 242: The KRAS protein of any one of embodiments 231 to 241, wherein the radiolabeled compound comprises a structure of Formula (Va), Formula (Vb), Formula (Vc).

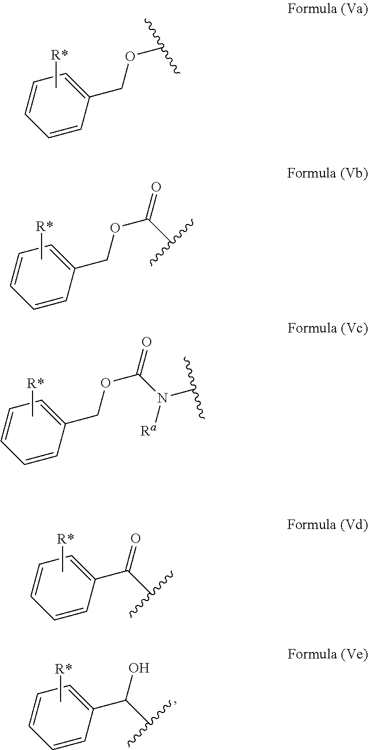

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At); and
R^{a} is hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₄heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl, or heteroaryl is optionally substituted.

Embodiment 243: The KRAS protein of embodiment 242, wherein the alkyl, haloalkyl, hydroxyalkyl, aminoalkyl, heteroalkyl, alkenyl, alkynyl, cycloalkyl, heterocycloalkyl, aryl, or heteroaryl is optionally substituted with one or more substituents selected from halogen, amino, —OH, —NO₂, oxo, —CN, C₁₋₃alkoxyl, C₁₋₃alkyl and C₁₋₃haloalkyl.

Embodiment 244: The KRAS protein of embodiment 242, wherein the radiolabeled compound comprises a structure of

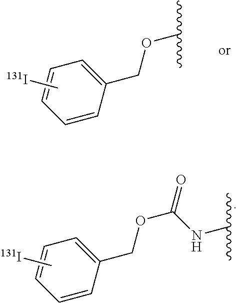

Embodiment 245: The KRAS protein of any one of embodiments 231 to 244, wherein the radiolabeled compound comprises a structure of Formula (IIIb):

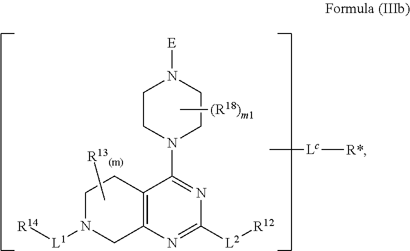

wherein
L^{C} is a linker comprising 1 to 20 groups independently selected from —CR^{b}R^{b}—, —C(═O)—, —S(O)—. —S(═O)₂, —NR^{a}—,

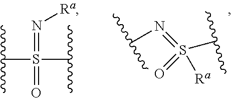

—CR^{b}═CR^{b}—, —C≡C—, —O—, —S—, —C(═O)O—, —OC(═O)—, —C(═O)NR^{a}—, —NR^{a}C(═O)—, —S(═O)₂NR^{a}—, —NR^{a}C(═O)₂—, —NR^{a}C(═O)NR^{a}—, —NR^{a}C(═O)O—, —OC(═O)NR^{a}—, arylene, heteroarylene;
each R^{a} is independently hydrogen, halogen, C₁-C₆alkyl, C₁-C₆haloalkyl, C₁-C₆hydroxyalkyl, C₁-C₆ aminoalkyl, C₁-C₆heteroalkyl, C₂-C₆alkenyl, C₂-C₆alkynyl, C₃-C₈cycloalkyl, C₂-C₈heterocycloalkyl, aryl, or heteroaryl;
each R^{b} is independently hydrogen, halogen, —CN, —NO₂, —OR^{a}, —SR^{a}, C₁-C₆alkyl, C₁-C₆haloalkyl, C₂-C₅ hydroxyalkyl, C₁-C₆aminoalkyl, C₂-C₆heteroalkyl, C₂-C₆alkenyl, C₂-C₆alkynyl, C₃-C₈cycloalkyl, C₂-C₉heterocycloalkyl, aryl, or heteroaryl;
R¹² is C₁-C₆ alkyl, C₁-C₆ heteroalkyl, -L³—NR^{a}—, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
R* is fluorine-18 (¹⁸F), iodine-131 (¹³¹I), iodine-123 (¹²³I), iodine-124 (¹²⁴I), iodine-125 (¹²⁵I), or astatine-211 (²¹¹ At); and
E, L¹, L², L³ R¹³, R¹⁴, R¹⁸, m and m1 have the meaning defined above in Formula (III).

Embodiment 246: The KRAS protein of embodiment 245, or a salt or solvate thereof, wherein the radiolabeled compound comprises a structure of Formula (IIIc):

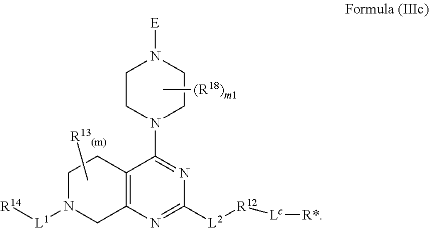

Embodiment 247: The KRAS protein of embodiment 245 or 240, wherein L^{C} comprises

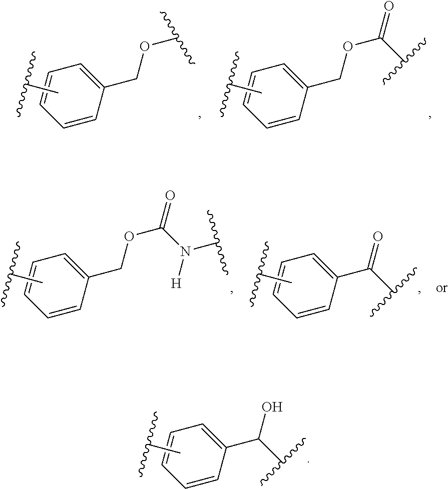

Embodiment 248: The KRAS protein of embodiment 245 or 246, wherein L^{C} comprises

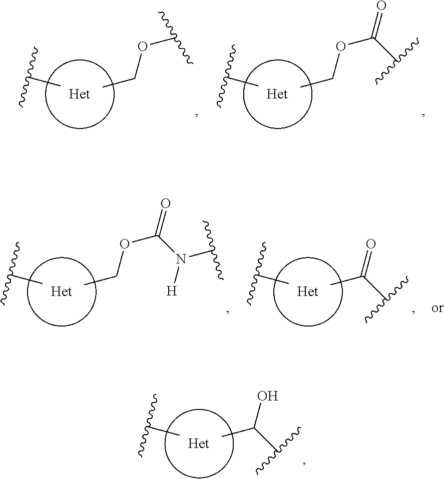

wherein Het is a 5-6 membered heteroaryl ring containing 1-2 heteroatoms independently selected from N, S, and O.

Embodiment 249: The KRAS protein of embodiment 245 or 246, wherein the radiolabeled compound has a structure of:

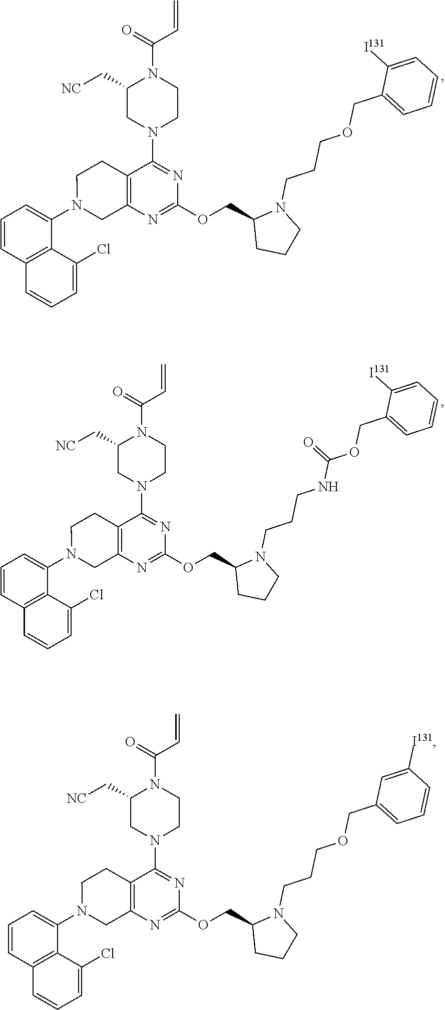

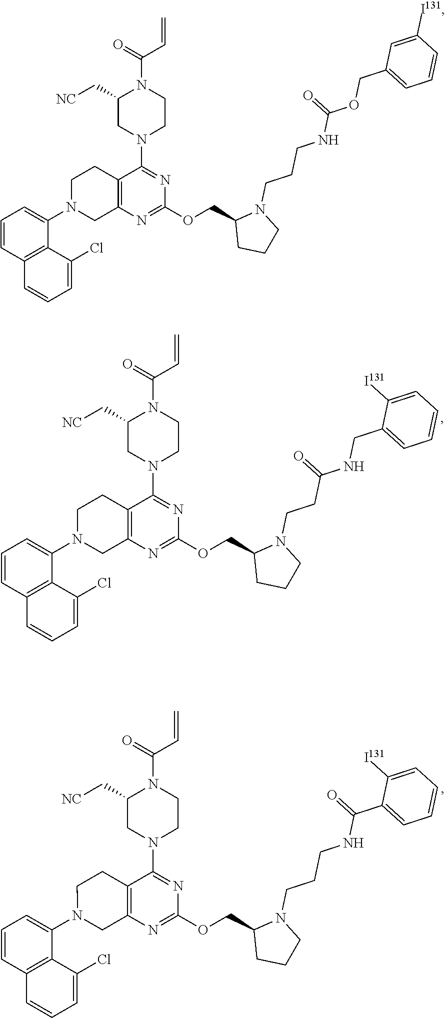

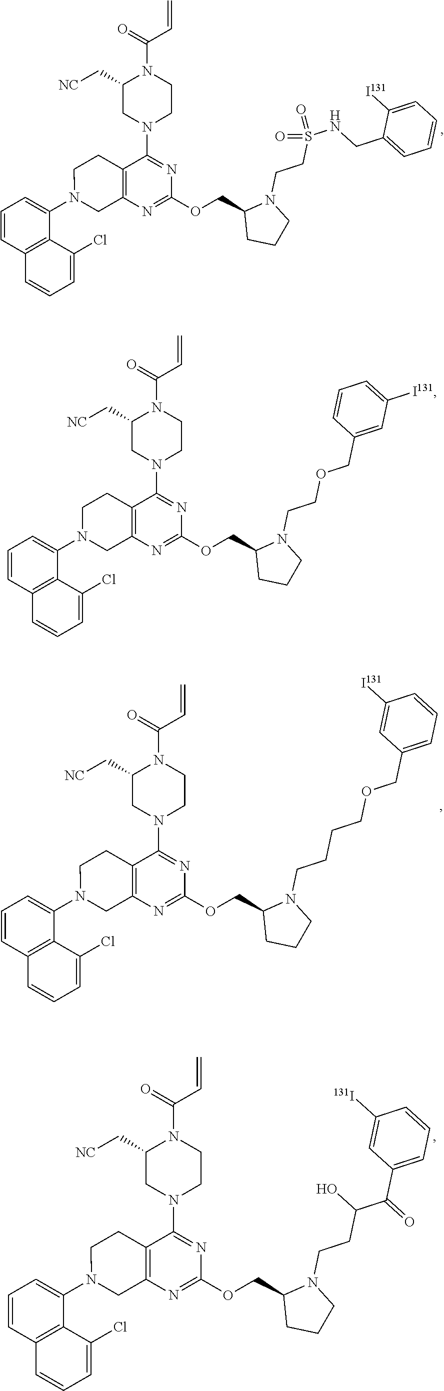

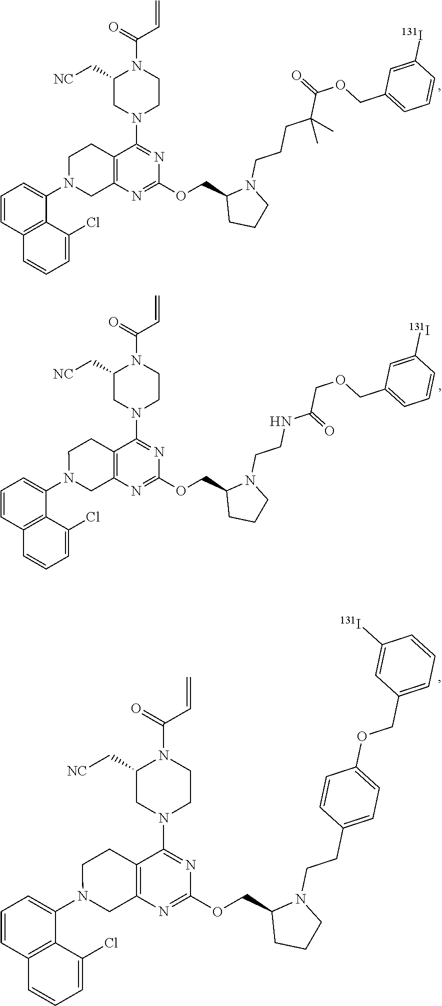 [SMILES: C=CC(=O)N1CCN(c2nc(OC[C@@H]3CCCN3CCCC(C)(C)C(=O)OCc3cccc([131I])c3)nc3c2CCN(c2cccc4cccc(Cl)c24)C3)C[C@@H]1CC#N ; C=CC(=O)N1CCN(c2nc(OC[C@@H]3CCCN3CCNC(=O)COCc3cccc([131I])c3)nc3c2CCN(c2cccc4cccc(Cl)c24)C3)C[C@@H]1CC#N ; C=CC(=O)N1CCN(c2nc(OC[C@@H]3CCCN3CCc3ccc(OCc4cccc([131I])c4)cc3)nc3c2CCN(c2cccc4cccc(Cl)c24)C3)C[C@@H]1CC#N]

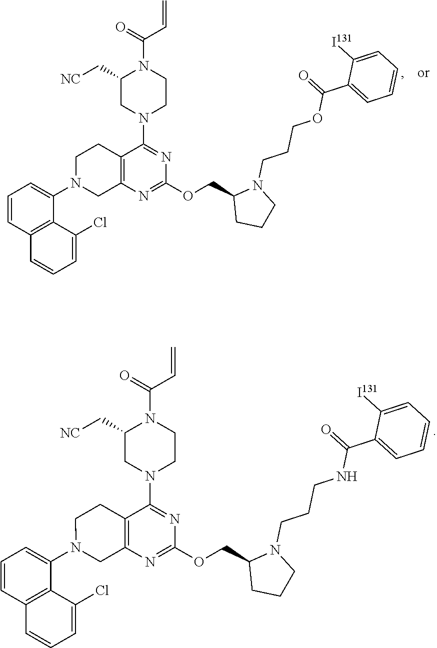

Embodiment 250: The KRAS protein of embodiment 245 or 246 wherein the radiolabeled compound has a structure of:

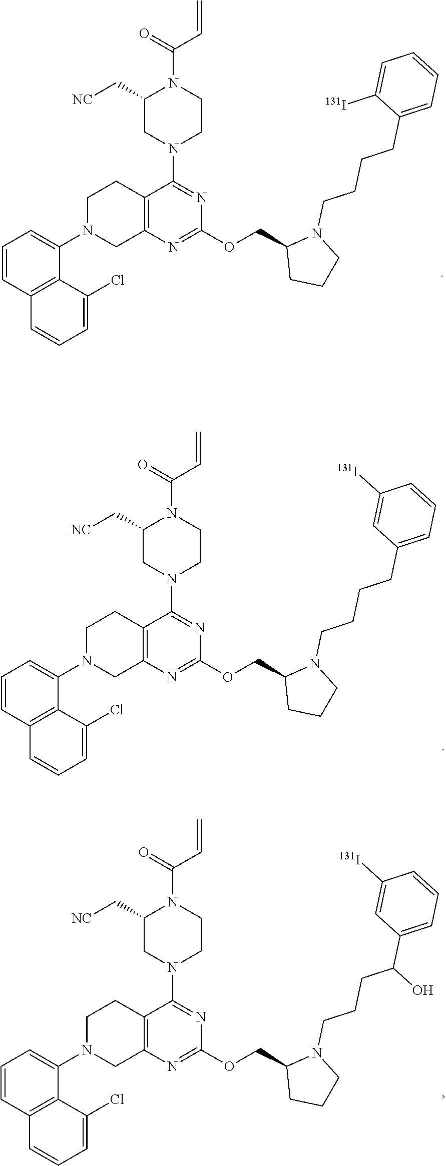 [SMILES: C=CC(=O)N1CCN(c2nc(OC[C@@H]3CCCN3CCCCc3ccccc3[131I])nc3c2CCN(c2cccc4cccc(Cl)c24)C3)C[C@@H]1CC#N ; C=CC(=O)N1CCN(c2nc(OC[C@@H]3CCCN3CCCCc3cccc([131I])c3)nc3c2CCN(c2cccc4cccc(Cl)c24)C3)C[C@@H]1CC#N ; C=CC(=O)N1CCN(c2nc(OC[C@@H]3CCCN3CCCC(O)c3cccc([131I])c3)nc3c2CCN(c2cccc4cccc(Cl)c24)C3)C[C@@H]1CC#N]

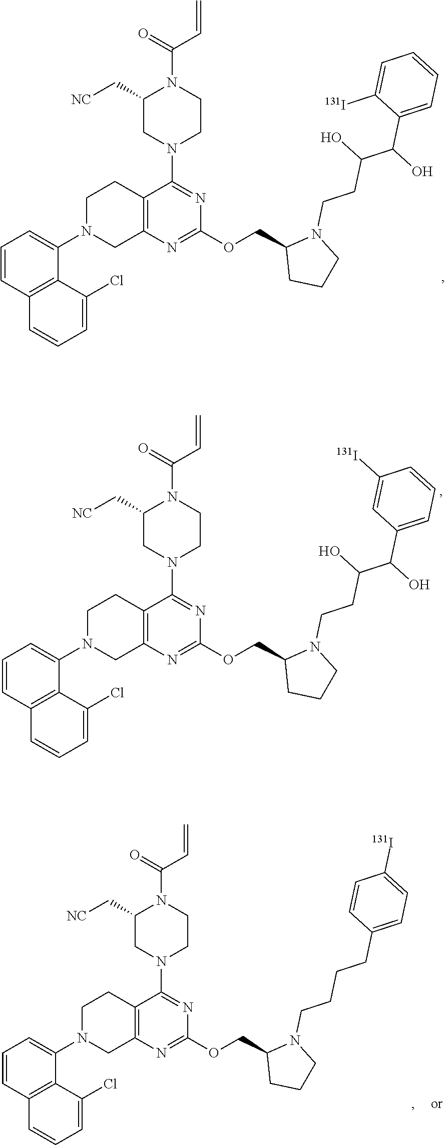 [SMILES: C=CC(=O)N1CCN(c2nc(OC[C@@H]3CCCN3CCC(O)C(O)c3ccccc3[131I])nc3c2CCN(c2cccc4cccc(Cl)c24)C3)C[C@@H]1CC#N ; C=CC(=O)N1CCN(c2nc(OC[C@@H]3CCCN3CCC(O)C(O)c3cccc([131I])c3)nc3c2CCN(c2cccc4cccc(Cl)c24)C3)C[C@@H]1CC#N ; C=CC(=O)N1CCN(c2nc(OC[C@@H]3CCCN3CCCCc3ccc([131I])cc3)nc3c2CCN(c2cccc4cccc(Cl)c24)C3)C[C@@H]1CC#N]

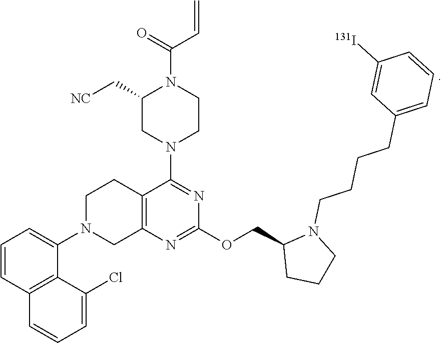 [SMILES: C=CC(=O)N1CCN(c2nc(OC[C@@H]3CCCN3CCCCc3cccc([131I])c3)nc3c2CCN(c2cccc4cccc(Cl)c24)C3)C[C@@H]1CC#N]

Embodiment 251: The KRAS protein of any one of embodiments 143 to 148, wherein the radiolabeled compound comprises a structure of Formula (IV), or a salt or solvate thereof,

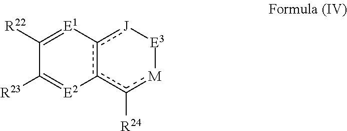

wherein
E¹ and E² are each independently N or CR²¹;
E³ is CR²⁸R²⁹, C═CR²⁸R²⁹, C═O, C═S, or C═NR²⁸;
J is N, NR³⁰, or CR³⁰;
M is N, NR³³, or CR³³;
[symbol] is a single or double bond as necessary to give every atom its normal valence;
R²¹ is independently H, hydroxyl, cyano, halogen, C₁-C₆ alkyl, C₁-C₆ alkoxyl, or C₁-C₆ heteroalkyl, wherein each of the alkyl, alkoxy, and heteroalkyl are optionally substituted;
R²² is hydrogen, halogen, C₁-C₆ alkyl, C₁-C₆ alkenyl, C₂-C₆ alkynyl, OR²². N(R²)₂, cycloalkyl, —C₁-C₃alkylene-cycloalkyl, heterocycloalkyl, —C₁-C₃alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted, and each R²² is independently H, C₁-C₆ alkyl, cycloalkyl, heterocycloalkyl, C₂-C₆ alkenyl, C₁-C₆ alkynyl, aryl, or heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form an optionally substituted 3-7-membered ring;
R²³ is hydrogen, halogen, C₁-C₆ alkyl, C₁-C₃ alkoxy, C₂-C₆ alkenyl, C₂-C₆ alkynyl, cycloalkyl, heterocycloalkyl, aryl, heteroaryl, -L⁴-cycloalkyl, -L⁴-heterocycloalkyl, -L⁴-aryl, or -L⁴-heteroaryl, wherein each of the alkyl, alkoxy, cycloalkyl, heterocycloalkyl, alkenyl, alkynyl, aryl and heteroaryl is optionally substituted;
L⁴ is C₁-C₄ alkylene or C₁-C₄ heteroalkylene, each of which is optionally substituted;
R²⁴ is

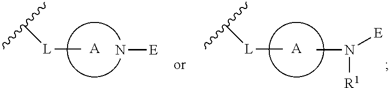

ring A is an optionally substituted 4-7 membered monocyclic ring or optionally substituted 6-11 membered bicyclic, bridged, fused, or Spiro ring;
R¹ is H, optionally substituted C₁-C₆ alkyl, optionally substituted C₁-C₆ heteroalkyl, optionally substituted C₃-C₆ cycloalkyl, or optionally substituted C—C₅ heterocycloalkyl;
L is a bond, S, O, or NR¹⁰, optionally substituted C₁-C₆ alkylene, or optionally substituted C₁-C₆ heteroalkylene;
E represents a structure of Formula (Ic),

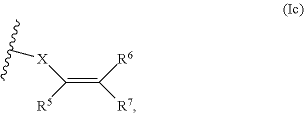

wherein,
X is C(═O), P(O)OR², C(═S), or S(═O)ₙ, where n is 1 or 2;
R² is H or optionally substituted C₁-C₃ alkyl;
R⁵ is hydrogen, cyano, halogen, C₁-C₆ alkyl, C₁-C₆ alkoxyl, C₁-C₆heteroalkyl, C₃-C₆ cycloalkyl, or C₂-C₄ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₁-C₃ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently H, hydroxyl, C₁-C₆ alkyl, C₁-C₆ alkoxy, cyano, nitro, C₃-C₆ cycloalkyl, or C₂-C₅ heterocycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, -L⁴-cycloalkyl, -L⁴-heterocycloalkyl, -L⁴-aryl, -L⁴-heteroaryl, —OR¹⁰, —SR¹⁰, —N(R¹⁰)(R^{10′}), —C(O)OR¹⁰, —OC(═O)N(R¹⁰)(R^{10′}), —N(R^{10′})C(O)N(R¹⁰)(R^{10′}), —N(R^{10′})C(O)OR¹⁵. —N(R^{10′})S(O)₂R¹¹, —C(O)R¹¹, —S(O)R¹¹, OC(═O)R¹¹, —C(O)N(R¹⁰)(R^{10′}), —C(O)C(O)N(R¹⁰)(R^{10′}), —N(R^{10′})C(O)R¹¹, —S(O)R¹¹, —S(O)₂N(R¹⁰)(R^{10′})—, S(═O)(═NH)N(R¹⁰)(R^{10′}), —CH₂C(O)N(R¹⁰)(R^{10′}), —CH₂N(R^{10′})C(O)R¹¹, —CH₂S(O)₂R¹¹, or —CH₂S(O)₂N(R¹⁰)(R^{10′}), wherein each of the alkyl, heteroalkyl, cycloalkyl, heterocycloalkyl, aryl, and heteroaryl is optionally substituted;
R³³ is H, C₁-C₆ alkyl, C₁-C₆ heteroalkyl, or C₃-C₆ cycloalkyl, wherein each of the alkyl, heteroalkyl, and cycloalkyl is optionally substituted;
each R¹⁰ is independently selected from hydrogen, C₁-C₆ alkyl, C₁-C₆ haloalkyl, C₂-C₆ alkenyl, C₂-C₆ alkynyl, C₃-C₆ cycloalkyl, C₂-C₉heterocycloalkyl, C₆-C₁₀ aryl, and C₁-C₉heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉ heteroaryl;
each R^{10′} is independently selected from hydrogen, C₁-C₆ alkyl, and C₁-C₆ haloalkyl; and
each R¹¹ is independently selected C₁-C₆ alkyl, C₂-C₆ alkenyl, C₂-C₆ alkynyl, C₁-C₆cycloalkyl, C₂-C₉ heterocycloalkyl, C₆-C₁₀ aryl, and C₁-C₉heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆, cycloalkyl, C₁-C₆ heterocycloalkyl, C₁-C₆ aryl, and C₁-C₉ heteroaryl.

Embodiment 252: The KRAS protein of embodiment 251, wherein
E¹ and E² are each independently Nor CR²¹;
E³ is CR²⁸R²⁹, C═CR²⁸R²⁹, C═O, C═S, or C═NR²⁸;
J is N, NR³⁰, or CR³⁰;
M is N, NR³³, or CR³⁰;
[symbol] is a single or double bond as necessary to give every atom its normal valence;
R²¹ is independently H, hydroxyl, cyano, halogen, C₁-C₆ alkyl, C₁-C₆ alkoxyl, or C₁-C₆ heteroalkyl, wherein each of the alkyl, alkoxy, and heteroalkyl is optionally substituted with one, two, or three groups selected from halogen, —CN, —NO₂, amino, hydroxy, C₁-C₆ alkyl, C₁-C₅ haloalkyl, C₁-C₆ alkoxy, C₂-C₆cycloalkyl, C₂-C₅heterocycloalkyl, C₆-C₁₀ aryl, and C₁-C₉ heteroaryl;
R²² is hydrogen, halogen, C₁-C₆ alkyl, C₂-C₆ alkenyl, C₂-C₆ alkynyl, OR^{22′}, N(R^{22′})₂, cycloalkyl, —C₁-C₃alkylene-cycloalkyl, heterocycloalkyl, —C₁-C₃alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted with one, two, or three groups selected from halogen, —CN, —NO₂, amino, hydroxy, C₁-C₆ alkyl, C₁-C₆, haloalkyl, C₁-C₆ alkoxy, C₃-C₆cycloalkyl, and C₂-C₅heterocycloalkyl, and each R^{22′} is independently H, C₁-C₆ alkyl, cycloalkyl, heterocycloalkyl, C₂-C₆ alkenyl, C₂-C₀ alkynyl, aryl, or heteroaryl, wherein each of the alkyl, cycloalkyl, heterocycloalkyl, alkenyl, alkyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, C₂-C₅ heterocycloalkyl, C₆-C₁₀ aryl, and C₁-C₉heteroaryl, or two R²² together with the nitrogen atom to which they are attached, form an optionally substituted 3-7-membered ring with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, C₂-C₅ heterocycloalkyl, C₆-C₁₀ aryl, and C₁-C₉heteroaryl;
R²³ is hydrogen, halogen, C₁-C₆ alkyl, C₁-C₆ alkoxy, C₂-C₆ alkenyl, C₂-C₆ alkynyl, cycloalkyl, heterocycloalkyl, aryl, heteroaryl, -L⁴-cycloalkyl, -L⁴-heterocycloalkyl, -L⁴-aryl, or -L⁴-heteroaryl, wherein each of the alkyl, alkoxy, cycloalkyl, heterocycloalkyl, alkenyl, alkynyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, C₂-C₅heterocycloalkyl, C₆-C₁₀ aryl, and C₁-C₆ heteroaryl;
L⁴ is C₁-C₄ alkylene or C₁-C₄ heteroalkylene, each of which is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉ heteroaryl;
   R²⁴ is

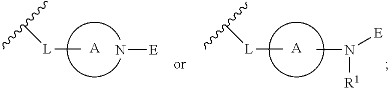

ring A is an optionally substituted 4-7 membered monocyclic ring or optionally substituted 6-11 membered bicyclic, bridged, fused, or Spiro ring each of which is optionally substituted with one, two, or three groups selected from halogen, —CN, —NO₂, amino, hydroxy, C₁-C₆ alkyl, C₁-C₅ haloalkyl, C₁-C₆ alkoxy, C₃-C₆cycloalkyl, C₂-C₅heterocycloalkyl, C₆-C₁₀ aryl, and C₁-C₉ heteroaryl;
R¹ is H, optionally substituted C₁-C₆ alkyl, optionally substituted C₁-C₆ heteroalkyl, optionally substituted C₃-C₆ cycloalkyl, or optionally substituted C₂-C₆ heterocycloalkyl each of which is optionally substituted with one, two, or three groups selected from halogen, —CN, —NO₂, amino, hydroxy, C₁-C₆ alkyl, C₁-C₆ haloalkyl, C₁-C₆ alkoxy, C₁-C₆cycloalkyl, C₂-C₅ heterocycloalkyl, C₆-C₁₀ aryl, and C₁-C₉heteroaryl;
L is a bond, S, O, or NR¹⁰, optionally substituted C₁-C₆ alkylene, or optionally substituted C₁-C₆ heteroalkylene with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉heteroaryl;
E represents a structure of Formula (IC),

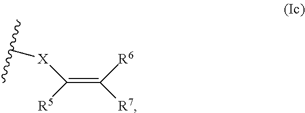

wherein,
X is C(═O), P(O)OR⁼, C(═S), or S(═O)ₙ, where n is 1 or 2;
R² is H or substituted or unsubstituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₅ heterocycloalkyl, C₆-C₁₀ aryl, and C₁-C₉ heteroaryl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₅ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₅ heterocycloalkyl, C₆-C₁₀ aryl, and C₁-C₉heteroaryl;
R⁷ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉heteroaryl; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉heteroaryl;
each R¹⁷ is independently halogen, hydroxyl, C₁-C₆ alkyl, C₃-C₆ cycloalkyl, C₁-C₆ alkoxy, C₁-C₆ haloalkyl, amino, cyano, C₁-C₆ heteroalkyl, C₁-C₆ hydroxyalkyl, —O—C₁-C₆haloalkyl, or —S—C₁-C₆haloalkyl;
R²⁸ and R²⁹ are each independently H, hydroxyl, C₁-C₆ alkyl, C₁-C₆ alkoxy, cyano, nitro, C₃-C₆ cycloalkyl, or C₂-C₆ heterocycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, -L³-cycloalkyl, -L⁴-heterocycloalkyl, -L⁴-aryl, -L⁴-heteroaryl, —OR¹⁰, —SR¹⁰, —N(R¹⁰)(R^{10′}), —C(O)OR¹⁰, —OC(═O)N(R(R¹⁰), —N(R^{10′})C(O)N(R¹⁰)(R^{10′}), —N(R)C(O)OR¹⁵, —N(R)S(O)₂R¹¹, —C(O)R¹¹, —S(O)R¹¹, —OC(═O)R¹¹, —C(O)N(R¹⁰)(R^{10′}), —C(O)C(O)N(R¹⁰)(R^{10′}), —N(R^{10′}), —S(O)₂R¹¹, —S(O)₂N(R¹⁰)(R^{10′})—, S(═O)(═NH)N(R¹⁰)(R^{10′}), —CH₂C(O)N(R¹⁰)(R^{10′}), —CH₂N(R^{10′})C(O)R¹¹, —CH₂S(O)₂R¹¹, or —CH₂S(O)₂N(R¹⁰)(R^{10′}), wherein each of the alkyl, heteroalkyl, cycloalkyl, heterocycloalkyl, aryl, and heteroaryl is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₆ heterocycloalkyl, C₆-C₁₀ aryl, and C₁-C₉ heteroaryl;
R³³ is H, C₁-C₆ alkyl, C₁-C₆ heteroalkyl, or C₁-C₆ cycloalkyl, wherein each of the alkyl, heteroalkyl, and cycloalkyl is optionally substituted with one, two, or three groups selected from halogen, —CN, hydroxy, C₁-C₆ alkyl, C₁-C₆ haloalkyl, C₁-C₆ alkoxy, C₁-C₆cycloalkyl, C₂-C₅heterocycloalkyl, C₆-C₁₀ aryl, and C₁-C₆heteroaryl;
each R¹⁰ is independently selected from hydrogen, C₁-C₆ alkyl, C₁-C₆ haloalkyl, C₂-C₆ alkenyl, C₂-C₆ alkynyl, C₃-C₆ cycloalkyl, C₂-C₉heterocycloalkyl, C₆-C₁₀ aryl, and C₁-C₆ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉heteroaryl,
each R^{10′} is independently selected from hydrogen, C₁-C₆ alkyl, and C₁-C₆ haloalkyl; and
each R¹¹ is independently selected C₁-C₆ alkyl, C₂-C₆ alkenyl. G-C₆ alkynyl, C₃-C₆cycloalkyl, C₂-C₉ heterocycloalkyl, C₅-C₁₀ aryl, and C₁-C₉heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₆ heterocycloalkyl, C₆-C₁₀ aryl, and C₁-C₉heteroaryl.

Embodiment 253: The KRAS protein of embodiment 251 or 252, wherein at least one of R²¹, R²², R²³, R²⁴, and R³⁰ comprises the covalently bonded radioisotope.

Embodiment 254: The KRAS protein of any one of embodiments 251 to 253, wherein the covalently bonded radioisotope is R* and R* is fluorine-18 (¹⁸F), iodine-131 (¹³¹I), iodine-123 (¹²³I), iodine-124 (¹²⁴I), iodine-125 (¹²⁵I), or astatine-211 (²¹¹At).

Embodiment 255: The KRAS protein of any one of embodiments 251 to 253, wherein the covalently bonded radioisotope is R* and R* is iodine-131 (¹³¹I).

Embodiment 256: The KRAS protein of embodiment 251 to 255, wherein the radiolabeled compound comprises a structure of Formula (IVa), or a salt or solvate thereof,

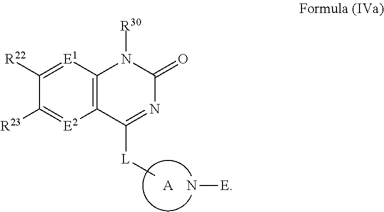

Embodiment 257: The KRAS protein of any one of embodiments 251 to 256 wherein the radiolabeled compound comprises a structure of Formula (IVb), or a salt or solvate thereof,

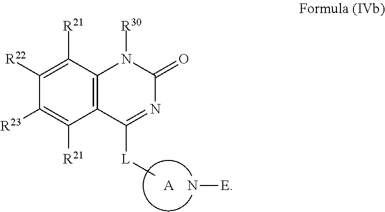

Embodiment 258: The KRAS protein of any one of embodiments 251 to 256, wherein the radiolabeled compound comprises a structure of Formula (IVc), or a salt or solvate thereof,

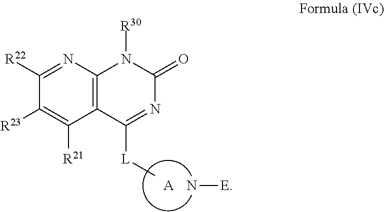

Embodiment 259: The KRAS protein of any one of embodiments 251 to 258, wherein each R²¹ is independently H, hydroxyl, cyano, halogen, C₁-C₆alkyl, C₁-C₄haloalkyl, C₁-C₄alkoxyl, or C₁-C₄ heteroalkyl.

Embodiment 26th: The KRAS protein of any one of embodiments 251 to 259, wherein R²¹ is H.

Embodiment 261: The KRAS protein of any one of embodiments 251 to 260, wherein R²² is halogen, C₁-C₆alkyl, C₂-C₃alkenyl, C₂-C₃alkynyl, OR^{22′}, N(R^{22′})₂, C₁-C₆cycloalkyl, C₂-C₅heterocycloalkyl, C₆-C₁₄aryl, or C₂-C₁₄heteroaryl, each of the alkyl, alkenyl, alkynyl, cycloalkyl, heterocycloalkyl, aryl, and heteroaryl is optionally substituted, and each R^{22′} is independently H, C₁-C₆alkyl, C₃-C₆cycloalkyl, C₂-C₅heterocycloalkyl, C₂-C₃alkenyl, C₂-C₃alkynyl, C₆-C₁₄aryl, C₂-C₁₄heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form an optionally substituted 3-7-membered ring.

Embodiment 262: The KRAS protein of any one of embodiments 251 to 261, wherein R²² is C₆-C₁₄aryl or C₂-C₁₄heteroaryl, each of which is optionally substituted.

Embodiment 263: The KRAS protein of any one of embodiments 251 to 262, wherein R²² is phenyl, optionally substituted with one or more substituents selected from C₁-C₃alkyl, halogen, and hydroxyl.

Embodiment 264: The KRAS protein of any one of embodiments 251 to 262, wherein R²² is bicyclic heteroaryl, optionally substituted with one or more substituents selected from C₁-C₃alkyl, halogen, and hydroxyl.

Embodiment 265: The KRAS protein of any one of embodiments 251 to 262, wherein R²² comprises a covalently bonded radioisotope R*.

Embodiment 266: The KRAS protein of embodiment 265, wherein R²² is

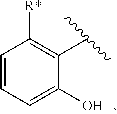

### and R* is ¹³¹I.

Embodiment 267: The KRAS protein of any one of embodiments 251 to 266, wherein R²³ is H, halogen, C₁-C₆ alkyl, C₁-C₃alkoxy, C₃-C₆cycloalkyl, C₂-C₅heterocycloalkyl, C₂-C₃alkenyl, C₂-C₃alkynyl, C₆-C₁₄aryl, or C₂-C₁₄heteroaryl, wherein each of the alkyl, alkoxy, cycloalkyl, heterocycloalkyl, alkenyl, alkynyl, aryl, and heteroaryl is optionally substituted.

Embodiment 268: The KRAS protein of any one of embodiments 251 to 267, wherein R²¹ is halogen, C₁-C₃alkyl, C₁-C₃haloalkyl.

Embodiment 269: The KRAS protein of any one of embodiments 251 to 268, wherein R²³ is halogen and the halogen is a radioisotope selected from fluorine-18 (¹⁸F), iodine-131 (¹³¹I), iodine-123 (¹²³I), iodine-124 (¹²⁴I), iodine-125 (¹²⁵I), and astatine-211 (²¹¹At).

Embodiment 270: The KRAS protein of any one of embodiments 251 to 269, wherein R²³ is ¹³¹I.

Embodiment 271: The KRAS protein of any one of embodiments 251 to 255 or 259 to 270, wherein R²⁴ is

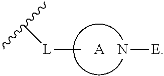

Embodiment 272: The KRAS protein of any one of embodiments 251 to 271, wherein ring A is an optionally substituted 4-7 membered monocyclic ring.

Embodiment 273: The KRAS protein of any one of embodiments 251 to 272, wherein ring A is an optionally substituted 6 membered heterocyclic ring.

Embodiment 274: The KRAS protein of any one of embodiments 251 to 273, wherein ring A is piperazinyl substituted with one to three substituents selected from halogen and C₁-C₃alkyl.

Embodiment 275: The KRAS protein of any one of embodiments 251 to 274, wherein L is a bond, C₁-C₃alkylene, S, O, or NH.

Embodiment 276: The KRAS protein of any one of embodiments 251 to 275, wherein L is a bond, CH₂, O, or NH.

Embodiment 277: The KRAS protein of any one of embodiments 251 to 276, wherein L is a bond.

Embodiment 278: The KRAS protein of any one of embodiments 251 to 277, wherein X is C(═O).

Embodiment 279: The KRAS protein of any one of embodiments 251 to 278, wherein R⁵ is hydrogen, cyano, halogen, C₁-C₆ alkyl, C₁-C₆ alkoxyl, C₁-C₆ heteroalkyl, C₃-C₆ cycloalkyl, or C₂-C₆ heterocycloalkyl, wherein each of the alkyl, alkoxy, heteroalkyl, cycloalkyl and heterocycloalkyl is optionally substituted.

Embodiment 280: The KRAS protein of any one of embodiments 251 to 279, wherein R⁵ is hydrogen, halogen, or a C₁-C₃alkyl optionally substituted by one or more hydroxyl and/or halogen.

Embodiment 281: The KRAS protein of embodiment 280, wherein R⁵ is a halogen.

Embodiment 282: The KRAS protein of embodiment 280, wherein R⁵ is a fluorine.

Embodiment 283: The KRAS protein of embodiment 280, wherein R⁵ is a hydrogen.

Embodiment 284: The KRAS protein of any one of embodiments 251 to 283, wherein R⁷ is hydrogen, cyano, halogen, optionally substituted C₁-C₆ alkyl, optionally substituted C₁-C₆ alkoxyl, or optionally substituted C₁-C₆ heteroalkyl.

Embodiment 285: The KRAS protein of any one of embodiments 251 to 284, wherein R⁷ is hydrogen.

Embodiment 286: The KRAS protein of any one of embodiments 251 to 278, wherein R⁵ and R⁷ taken together with the carbon atoms to which they are attached form a 5-8 membered partially saturated cycloalkyl, wherein the cycloalkyl is optionally substituted with one or more R¹⁷, wherein each R¹⁷ is independently halogen, hydroxyl, C₁-C₆ alkyl, C₁-C₆ cycloalkyl, C₁-C₆ alkoxy, C₁-C₆haloalkyl, amino, cyano, C₁-C₆ heteroalkyl, C₁-C₆ hydroxyalkyl, —O—C₁-C₆haloalkyl, or —S—C₁-C₆haloalkyl.

Embodiment 287: The KRAS protein of any one of embodiments 251 to 286, wherein R⁶ is hydrogen, cyano, halogen, optionally substituted C₁-C₆ alkyl, optionally substituted C₁-C₆ alkoxyl, optionally substituted C₁-C₆ heteroalkyl, optionally substituted C₃—C cycloalkyl, or optionally substituted C₂-C₆, heterocycloalkyl.

Embodiment 288: The KRAS protein of any one of embodiments 251 to 287, wherein R⁶ is hydrogen.

Embodiment 289: The KRAS protein of any one of embodiments 251 to 255 or 259 to 288, wherein E³ is C═O.

Embodiment 290: The KRAS protein of any one of embodiments 251 to 289, wherein R³⁰ is halogen, cyan, C₁-C₆ alkyl, C₁-C₆ alkoxy, C₁-C₆ heteroalkyl, cycloalkyl, heterocycloalkyl, aryl, heteroaryl, -L⁴-cycloalkyl, -L³-heterocycloalkyl, -L³-aryl, -L⁴-heteroaryl, —OR¹⁰, —SR¹⁰, wherein each of the alkyl, heteroalkyl, cycloalkyl, heterocycloalkyl, aryl, and heteroaryl is optionally substituted.

Embodiment 291: The KRAS protein of any one of embodiments 251 to 290, wherein R³⁰ is C₆-C₁₄aryl, optionally substituted with one or more substituents selected from halogen, C₁-C₃alkyl, C₁-C₃alkoxyl, and cyano.

Embodiment 292: The KRAS protein of any one of embodiments 251 to 290, wherein R³⁰ is C₂-C₁₄heteroaryl, optionally substituted with one or more substituents selected from halogen, C₁-C₃alkyl, C₁-C₃alkoxyl, or cyano.

Embodiment 293: The KRAS protein of any one of embodiments 251 to 290, wherein R¹⁹ is phenyl or 6-membered heteroaryl, optionally substituted with one or more of C₁-C₃alkyl.

Embodiment 294: The KRAS protein of any one of embodiments 251 to 255 or 259 to 293, wherein R³³ is H or C₂-C₅ alkyl.

Embodiment 295: The KRAS protein of any one of embodiments 251 to 258, wherein the radiolabeled compound has a structure of

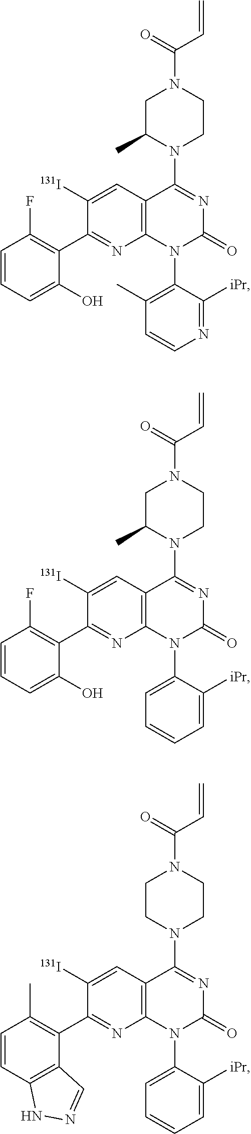 [SMILES: C=CC(=O)N1CCN(c2nc(=O)n(-c3c(C)ccnc3C(C)C)c3nc(-c4c(O)cccc4F)c([131I])cc23)[C@@H](C)C1 ; C=CC(=O)N1CCN(c2nc(=O)n(-c3ccccc3C(C)C)c3nc(-c4c(O)cccc4F)c([131I])cc23)[C@@H](C)C1 ; C=CC(=O)N1CCN(c2nc(=O)n(-c3ccccc3C(C)C)c3nc(-c4c(C)ccc5[nH]ncc45)c([131I])cc23)CC1]

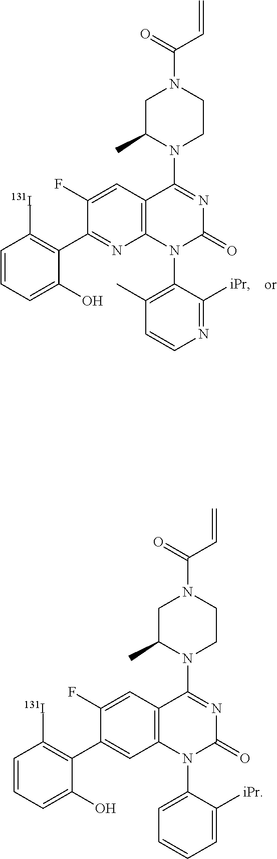 [SMILES: C=CC(=O)N1CCN(c2nc(=O)n(-c3c(C)ccnc3C(C)C)c3nc(-c4c(O)cccc4[131I])c(F)cc23)[C@@H](C)C1 ; C=CC(=O)N1CCN(c2nc(=O)n(-c3ccccc3C(C)C)c3cc(-c4c(O)cccc4[131I])c(F)cc23)[C@@H](C)C1]

Embodiment 2%: The KRAS protein of any one of embodiments 251-264.267, or 271-294, wherein the radiolabeled compound comprises;
a) the structure of Formula (IV) or a salt or solvate thereof;
b) the covalently bonded radioisotope; and
c) a linker covalently bonded to the structure of Formula (IV) or a salt or solvate thereof and to the radioisotope.

Embodiment 297: The KRAS protein of embodiment 296, wherein the linker comprises C₁-C₁₀) alkylene or C₁-C₁₀ heteroalkylene, wherein the alkylene or heteroalkylene is optionally substituted with one or more substituents selected from halogen, amino, —OH, —NO₂, oxo, —CN, C₁₋₃alkoxyl, C₁₋₃alkyl, C₁₋₃hydroxyalkyl, C₁₋₃aminoalkyl, and C₁₋₃haloalkyl.

Embodiment 298: The KRAS protein of embodiment 297, wherein the linker comprises C₁-C₆ alkylene.

Embodiment 299: The KRAS protein of any one of embodiments 296 to 298, wherein the linker comprises one or more functional groups selected from: —O—, —S—, —S—S—, —C(═O)—, —OC(═O)—, —C(═O)NR^{a}—, —NR^{a}C(═O)—, —S(═O)₂NR^{a}—, —NR^{a}C(═O)₂—, —NR^{a}C(═O)NR^{a}—, —NR^{a}C(O)O—, and —OC(═O)NR^{a}—,
wherein each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl.

Embodiment 300: The KRAS protein of any one of embodiments 296 to 299, wherein the linker comprises

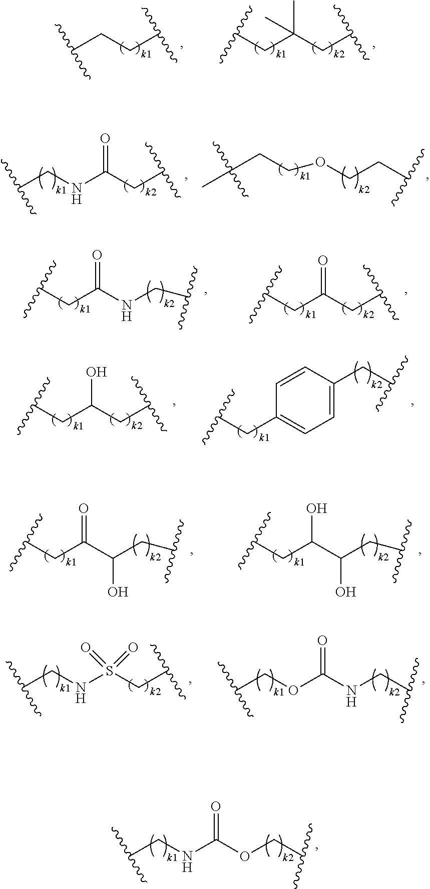

wherein each k1 and k2 is independently 0 or an integer selected from 1 to 10.

Embodiment 301: The KRAS protein of any one of embodiments 296 to 300, wherein the linker comprises

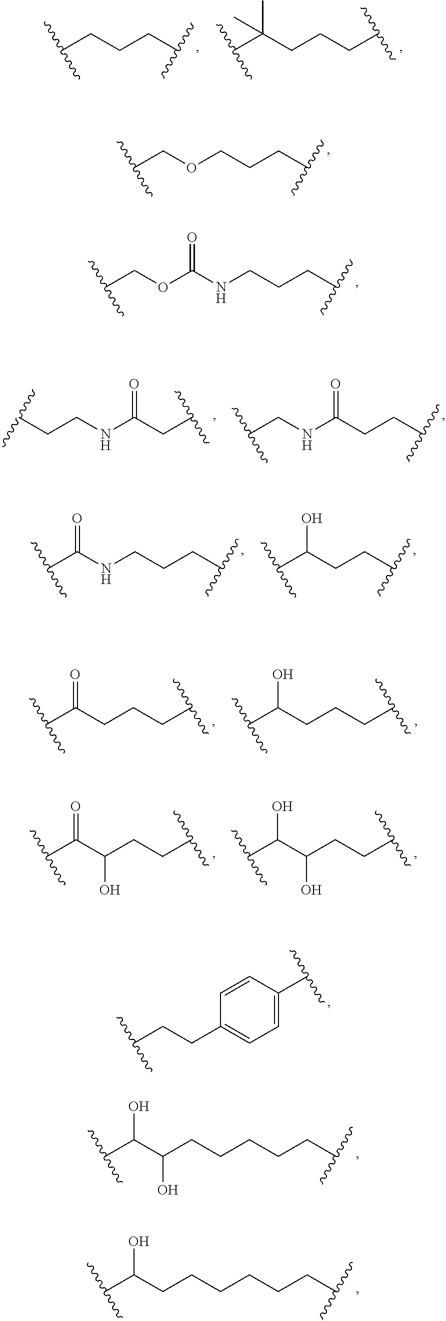

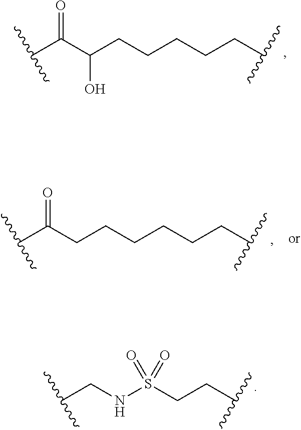 [SMILES: *CCCCCC(O)C(*)=O ; *CCCCCCC(*)=O ; *CCS(=O)(=O)NC*]

Embodiment 302: The KRAS protein of any one of embodiments 296 to 301, wherein the linker is a brush border enzyme-cleavable linker.

Embodiment 303: The KRAS protein of any one of embodiments 296 to 301, wherein the linker is a hepatocyte-cleavable linker.

Embodiment 304: The KRAS protein of any one of embodiments 2% to 301, wherein the linker is a cytochrome P450-substrate.

Embodiment 305: The KRAS protein of any one of embodiments 296 to 301, wherein the linker is an esterase-cleavable linker.

Embodiment 306: The KRAS protein of any one of embodiments 296 to 301, wherein the linker is a peptidase-cleavable linker.

Embodiment 307: The KRAS protein of any one of embodiments 296 to 306, wherein the radiolabeled compound comprises a structure of Formula (Va), Formula (Vb), Formula (Vc), Formula (Vd), or Formula (Ve):

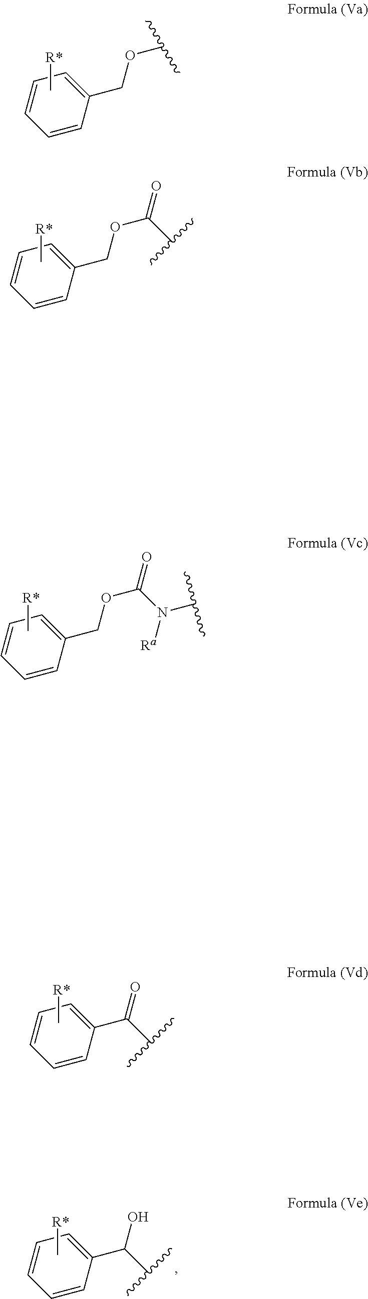

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At); and
R^{a} is hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₃-C₈alkynyl, C₃-C₆cycloalkyl, C₂-C₉heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl, or heteroaryl is optionally substituted.

Embodiment 308: The KRAS protein of embodiment 307, wherein the alkyl, haloalkyl, hydroxyalkyl, aminoalkyl, heteroalkyl, alkenyl, alkynyl, cycloalkyl, heterocycloalkyl, aryl, or heteroaryl is optionally substituted with one or more substituents selected from halogen, amino, —OH, —NO₂, oxo, —CN, C₁₋₃ alkoxyl, C₁₋₃ alkyl and C₁₋₃ haloalkyl.

Embodiment 309: The KRAS protein of embodiment 307, wherein the radiolabeled compound comprises a structure of

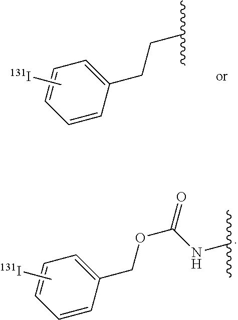

Embodiment 310: The KRAS protein of any one of embodiments 296 to 306, wherein the radiolabeled compound comprises a structure of Formula (IVd)

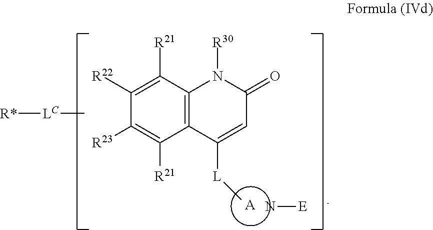

wherein
L^{C} is a linker comprising 1 to 20 groups independently selected from —CR^{b}R^{b}—, —C(═O)—, —S(O)—, —S(═O)₂—, —NR^{a}—,

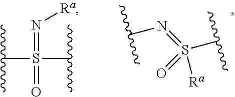

—CR^{b}═CR^{b}—, —C≡C—, —O—, —S—, —C(═O)O—, —OC(═O)—, —C(═O)NR^{a}—, —NR^{a}C(═O)—, —S(═O)₂NR^{a}—, —NR¹S(═O)₂—, —NR^{a}C(═O)NR^{a}—, —NR^{a}C(═O)O—, —OC(═O)NR^{a}—, arylene, heteroarylene;
each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
each R^{b} is independently hydrogen, halogen, —CN, —NO₂, —OR^{a}, —SR^{a}, C₁-C₆alkyl, C₁-C₆haloalkyl, C₁-C₆hydroxyalkyl, C₁-C₆aminoalkyl, C₁-C₄heteroalkyl, C₂-C₆alkenyl, C₂-C₆alkynyl, C₃-C₈cycloalkyl, C₂-C₉heterocycloalkyl, aryl, or heteroaryl;
R* is fluorine-18 (¹⁸F), iodine-131 (¹³¹I), iodine-123 (²¹¹I), iodine-124 (¹²⁴I), iodine-125 (¹²⁵I), or astatine-211 (²¹¹At);
R²² is C₁-C₆ alkyl, C₂-C₆ alkenyl, C₂-C₆ alkynyl, cycloalkyl, —C₁-C₃alkylene-cycloalkyl, heterocycloalkyl, —C₁-C₃alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted; and
E, ring A, L, R²¹, R²², R²³, and R³⁰ have the meaning defined above in Formula (IV).

Embodiment 311: The KRAS protein of 310, wherein the radiolabeled compound comprises a structure of Formula (IVe).

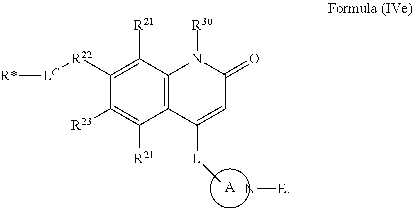

Embodiment 312: The KRAS protein of embodiment 310 or 311, wherein L^{C} comprises

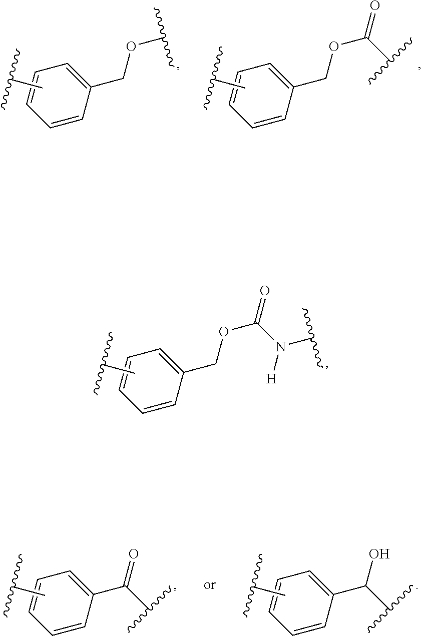

Embodiment 313: The KRAS protein of embodiment 310 or 311, wherein L^{C} comprises

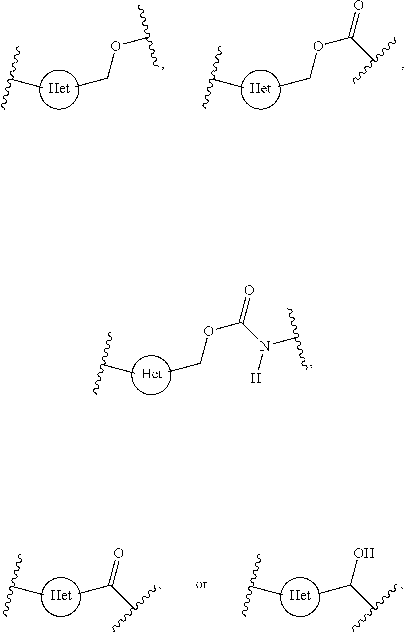

wherein Het is a 5-6 membered heteroaryl ring containing 1-2 heteroatoms independently selected from N, S, and O.

Embodiment 314: The KRAS protein of any one of embodiments 143 to 313, wherein the radiolabeled compound is in contact with one or more amino acid residues of a KRAS protein Switch 2 binding pocket.

Embodiment 315: A radiolabeled compound having a structure of Formula (III), or a salt or solvate thereof,

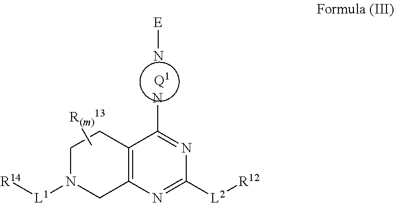

wherein
ring Q¹ is a 4-12 membered saturated or partially saturated monocyclic or bicyclic ring, wherein the monocyclic or bicyclic ring is optionally substituted;
L¹ is a bond, —C(═O)—, O, S, NR′, optionally substituted C₁-C₆ alkylene, optionally substituted C₁-C₃ heteroalkylene;
L² is a bond, —C(═O)—, O, S or NR¹⁵;

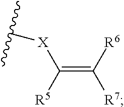

X is C(═O), P(O)OR², C(═S), or S(═O)ₙ, where n is 1 or 2;
R² is H or optionally substituted C₁-C₆ alkyl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆heteroalkyl, C₃-C₆ cycloalkyl, C₂-C₆ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl), each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl), each of the alkyl, alkylene, alkoxyl, heteroalkyl, cycloalkyl, heterocycloalkyl, or heteroaryl is optionally substituted;
R¹² is hydrogen, C₁-C₆ alkyl, C₁-C₆ heteroalkyl, -L³—NR¹⁵R^{15′}, heterocycloalkyl, -L³-heterocycloalkyl, cycloalkyl, -L-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted; each R¹³ is independently OH, halogen, oxo, substituted or unsubstituted C₁-C₆ alkyl, or substituted or unsubstituted C₁-C₆ heteroalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or optionally substituted C₁-C₃ alkyl;
each R^{15′} is independently hydrogen, acyl, optionally substituted C₁-C₆ alkyl, optionally substituted C₁-C₃ heteroalkyl or optionally substituted C₁-C₃ hydroxyalkyl; and
m is 0, 1, or 2;
provided that at least one of L¹, L², R¹², R¹³ and R¹⁴ comprises a covalently bonded radioisotope R*.

Embodiment 316: A radiolabeled compound comprising
(a) a structure of Formula (III), or a salt or solvate thereof,

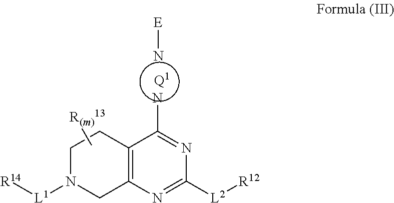

wherein
ring Q¹ is a 4-12 membered saturated or partially saturated monocyclic or bicyclic ring, wherein the monocyclic or bicyclic ring is optionally substituted;
L¹ is a bond, —C(═O)—, O. S. NR¹⁵, optionally substituted C₁-C₃ alkylene, optionally substituted C₁-C₆ heteroalkylene;
L² is a bond, —C(═O)—, O, S or NR¹⁵;

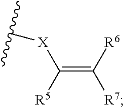

X is C(═O), P(═O)OR, C(═S), or S(O)ₙ, where n is 1 or 2;
R² is H or optionally substituted C₁-C₃ alkyl;
R⁵ is hydrogen, cyano, halogen, C₁-C₆ alkyl, C₁-C₆ alkoxyl, C₁-C₆ heteroalkyl, C₃-C₆, cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl), each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₂-C₅ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆ alkylene-NH(C₁-C₆ alkyl), or —C₁-C₆alkylene-N(C₁-C₆ alkyl), each of the alkyl, alkylene, alkoxyl, heteroalkyl, cycloalkyl, heterocycloalkyl, or heteroaryl is optionally substituted;
R¹² is hydrogen, C₁-C₆ alkyl, C₁-C₆ heteroalkyl, -L³—NR¹⁵R^{15′}, heterocycloalkyl, -L³-heterocycloalkyl, cycloalkyl, -L-cycloalkyl, aryl, heteroaryl, -L³-aryl, or -L-heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted;
each R¹³ is independently OH, halogen, oxo, substituted or unsubstituted C₁-C₆ alkyl, or substituted or unsubstituted C₁-C₆ heteroalkyl;
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or optionally substituted C₁-C₃ alkyl;
each R^{15′} is independently hydrogen, acyl, optionally substituted C₁-C₃ alkyl, optionally substituted C₁-C₃ heteroalkyl or optionally substituted C₁-C₃ hydroxyalkyl; and
m is 0, 1, or 2;
(b) a covalently bonded radioisotope R*; and
(c) a linker covalently connecting the radioisotope R* and the structure of Formula (III) or a salt or solvate thereof.

Embodiment 317: The radiolabeled compound of 316, or a salt or solvate thereof, wherein the radiolabeled compound comprises a structure of Formula (IIIb)

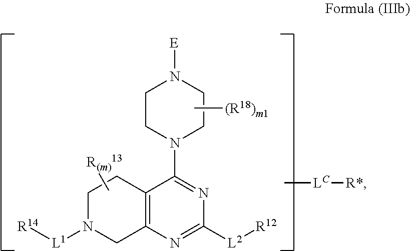

wherein
L^{C} is a linker comprising 1 to 20 groups independently selected from —CR^{b}R^{b}—, —C(═O)—, —S(O)—, —S(═O)₂—, —NR^{a}—

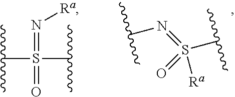

CR^{b}═CR^{b}, —C≡C—, —O—, —S—. —C(═O)O—, —OC(═O)—, —C(═O)NR^{a}—, —NR^{a}C(═O)—, —S(═O)₂NR^{a}—, —NR¹S(═O)₂—, —NR^{LK}C(═O)NR^{a}—, —NR^{a}C(═O)O—. —OC(═O)NR^{a}—, arylene, heteroarylene;
each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
each R^{b} is independently hydrogen, halogen, —CN, —NO₂, —OR^{a}, —SR^{a}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
each R¹⁸ is independently halogen, oxo, C₁-C₆, alkyl, C₁-C₆ aminoalkyl, C₁-C₆ haloalkyl, C₂-C₆ alkenyl, C₂-C₆ alkynyl, C₁-C₆ heteroalkyl, —CN, —C(O)OR¹⁵, —C(O)N(R¹⁵)(R^{15′}), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₁-C₅ haloalkyl, C₁-C₆ alkoxy, C₃-C₆cycloalkyl, and C₂-C₅heterocycloalkyl;
R¹² is C₁-C₆ alkyl, C₁-C₆ heteroalkyl, -L³—NR¹⁵—, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
m1 is 0. 1, 2, or 3;
R* is fluorine-18 (¹⁸F) iodine-131 (¹³¹I), iodine-123 (¹²³I), iodine-124 (¹²⁴I), iodine-125 (¹²⁵I), or astatine-211 (²¹¹At); and
E, L¹, L², R¹³, R¹⁴, and m have the meaning defined above in Formula (III).

Embodiment 318: The radiolabeled compound of embodiment 317, or a salt or solvate thereof, wherein the radiolabeled compound comprises a structure of Formula (IIIc),

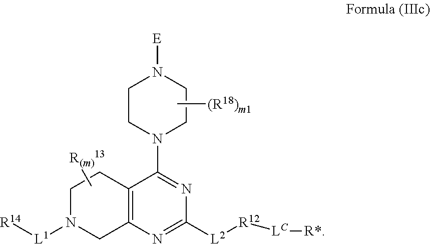

Embodiment 319: The radiolabeled compound of embodiment 317 or 318, wherein L^{C} comprises

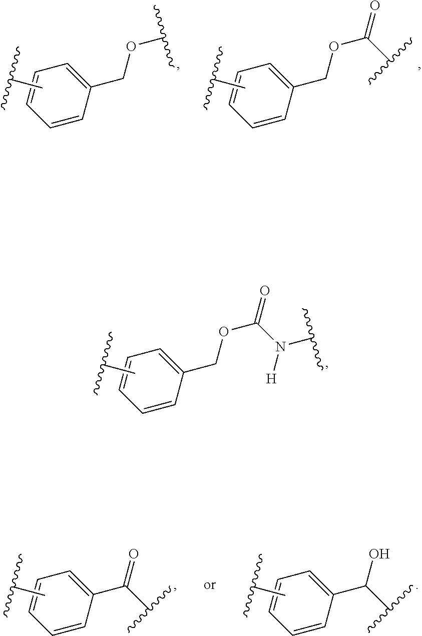

Embodiment 320: The KRAS protein of embodiment 317 or 318, wherein L^{C} comprises

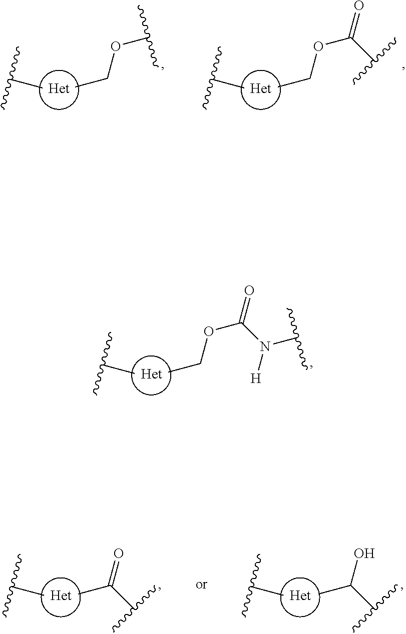

wherein Het is a 5-6 membered heteroaryl ring containing 1-2 heteroatoms independently selected from N, S, and O.

Embodiment 321: A radiolabeled compound comprising a structure of Formula (IV), or a salt or solvate thereof,

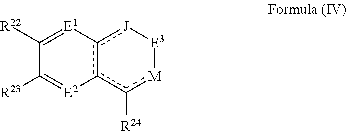

wherein
E¹ and E² are each independently N or CR²¹;
E³ is CR²⁸R²⁹, C═CR²⁸R²⁹, C═O, C═S, or C═NR²⁸;
J is N, NR³⁰, or CR³⁰;
M is N, NR³³, or CR³³;
[symbol] is a single or double bond as necessary to give even atom its normal valence;
R²¹ is independently H, hydroxyl, cyano, halogen, C₁-C₆ alkyl, C₁-C₆ alkoxyl, or C₁-C₆ heteroalkyl, wherein each of the alkyl, alkoxy, and heteroalkyl is optionally substituted;
R²² is hydrogen, halogen, C₁-C₆ alkyl, C₂-C₆alkenyl, C₂-C₆ alkynyl, OR¹⁵, N(R²²)₂, cycloalkyl, —C₁-C₃alkylene-cycloalkyl, heterocycloalkyl, —C₁-C₃alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted, and each R¹² is independently H, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted, or two R²² together with the nitrogen atom to which they are attached, form a 3-7-membered ring;
R²³ is hydrogen, halogen, C₁-C₆ alkyl, C₁-C₆ alkoxy, C₂-C₆ alkenyl, C₂-C₆ alkynyl, cycloalkyl, heterocycloalkyl, aryl, heteroaryl, -L⁴-cycloalkyl, -L³-heterocycloalkyl, -L⁴-aryl, or -L⁴-heteroaryl, wherein each of the alkyl, alkoxy, cycloalkyl, heterocycloalkyl, alkenyl, alkynyl, aryl, and heteroaryl is optionally substituted;
L⁴ is C₁-C₄ alkylene or C₁-C₄ heteroalkylene, each of which is optionally substituted;
R²⁴ is

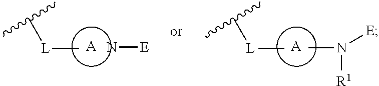

ring A is an optionally substituted 4-7 membered monocyclic ring or optionally substituted 6-11 membered bicyclic, bridged, fused, or spiro ring;
R¹ is H, optionally substituted C₁-C₆ alkyl, optionally substituted C₁-C₆ heteroalkyl, optionally substituted C₃-C₆ cycloalkyl, or optionally substituted C₂-C₅ heterocycloalkyl;
L is a bond, S, O, or NR^{10′}, optionally substituted C₁-C₆ alkylene, or optionally substituted C₁-C₆ heteroalkylene;
E represents a structure of Formula (Ic),

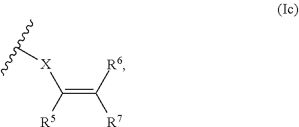

wherein,
X is C(═O), P(═O)OR², C(═S), or S(═O)ₙ, where n is 1 or 2;
R² is H or optionally substituted C₁-C₆ alkyl;
R⁵ is hydrogen, cyano, halogen, C₁-C₆ alkyl, C₁-C₆ alkoxyl, C₂-C₅ heteroalkyl, C₃-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₂-C₅ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl), each of the alkyl, alkylene, alkoxyl, heteroalkyl, cycloalkyl, heterocycloalkyl, or heteroaryl is optionally substituted;
R²⁸ and R²⁹ are each independently H, hydroxyl, C₁-C₆ alkyl, C₁-C₆ alkoxy, cyano, nitro, C₃-C₆, cycloalkyl, or C₂-C₅ heterocycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, -L⁴-cycloalkyl, -L⁴-heterocycloalkyl, -L⁴-aryl, -L⁴-heteroaryl, —OR¹⁰, —SR¹⁰, —N(R¹⁰)(R^{10′}), —C(O)OR¹⁰, —OC(═O)N(R¹⁰)(R^{10′}), —N(R^{10′})C(O)N(R¹⁰)(R^{10′}), —N(R¹⁰)C(O)OR¹¹, —N(R¹⁰)S(O)₂R¹¹, —C(O)R¹¹, —S(O)R¹¹, —OC(═O)R¹¹, —C(O)N(R¹⁰)(R^{10′}), —C(O)C(O)N(R¹⁰)(R^{10′}), —N(R¹⁰)C(O)R′ 1, —S(O)₂R¹¹. —S(O)₂N(R¹⁰)(R^{10′})—, S(═O)(═NH)N(R¹⁰)(R^{10′}), —CH—C(O)N(R¹⁰)(R^{10′}), —CH₂N(R^{10′})C(O)R¹¹, —CH₂S(O)₂R¹¹, or —CH₂S(O)₂N(R¹⁰)(R^{10′}), wherein each of the alkyl, heteroalkyl, cycloalkyl, heterocycloalkyl, aryl, and heteroaryl is optionally substituted;
R³³ is H, C₁-C₆ alkyl, C₁-C₆ heteroalkyl, or C₃-C₆ cycloalkyl, wherein each of the alkyl, heteroalkyl, and cycloalkyl is optionally substituted;
each R¹⁰ is independently selected from hydrogen, C₁-C₆, alkyl, C₁-C₆ haloalkyl, C₂-C₆ alkenyl, C₂-C₆ alkynyl, C₃-C₆ cycloalkyl, C₂-C₉heterocycloalkyl, C₆-C₁₀ aryl, and C₁-C₆ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₉heteroaryl;
each R^{10′} is independently selected from hydrogen, C₁-C₆ alkyl, and C₁-C₆ haloalkyl; and
each R¹¹ is independently selected C₁-C₆ alkyl, C₂-C₆ alkenyl, C₂-C₆ alkynyl, C₃-C₆ cycloalkyl, C₂-C₉ heterocycloalkyl, C₆-C₁₀ aryl, and C₁-C₃ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆, alkoxy, C₃-C₆ cycloalkyl, C₂-C₉ heterocycloalkyl, C₆-C₁₀ aryl, and C₁-C₉heteroaryl;
provided that at least one of R²¹, R²², R²³, R²⁴, and R³⁰ comprises a covalently bonded radioisotope R*.

Embodiment 322: A radiolabeled compound, comprising
(a) a structure of Formula (IV), or a salt or solvate thereof,

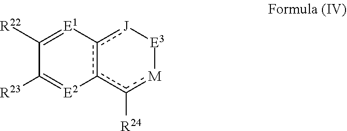

wherein
E¹ and E² are each independently N or CR²¹;
E³ is CR²⁸R²⁹, C═CR²⁸R²⁹, C═O, C═S, or C═NR²⁸;
J is N, NR³⁰, or CR³⁰;
M is N, NR³³, or CR³³;
[symbol] is a single or double bond as necessary to give every atom its normal valence;
R²¹ is independently H, hydroxyl, cyano, halogen, C₁-C₆ alkyl, C₁-C₆ alkoxyl, or C₁-C₆ heteroalkyl, wherein each of the alkyl, alkoxyl, and heteroalkyl is optionally substituted;
R²² is hydrogen, halogen, C₁-C₆ alkyl, C₂-C₆ alkenyl, G-C₆ alkynyl, OR², N(R)₂, cycloalkyl, —C₂-C₅ alkylene-cycloalkyl, heterocycloalkyl, —C₁-C₃alkylene-heterocycloalkyl, aryl, —C₁-C₃alkylene-aryl, heteroaryl, or —C₁-C₆ alkylene-heteroaryl, wherein each of the alkyl, alkylene, alkenyl, alkynyl, cycloalkyl, heterocycloalkyl, aryl, and heteroaryl is optionally substituted, and each R^{22′} is independently H, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form a 3-7-membered ring;
R²³ is hydrogen, halogen, C₁-C₆ alkyl, C₁-C₆ alkoxy, C₁-C₆, alkenyl, C₂-C₆ alkynyl, cycloalkyl, heterocycloalkyl, aryl, heteroaryl, -L³-cycloalkyl, -L⁴-heterocycloalkyl, -L³-aryl, or -L³-heteroaryl, wherein each of the alkyl, alkoxy, cycloalkyl, heterocycloalkyl, alkenyl, alkynyl, aryl, and heteroaryl is optionally substituted;
L⁴ is C₁-C₄ alkylene or C₁-C₄ heteroalkylene, each of which is optionally substituted;
R²⁴ is

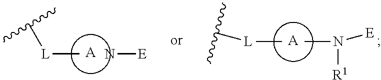

ring A is an optionally substituted 4-7 membered monocyclic ring or optionally substituted 6-11 membered bicyclic, bridged, fused, or spiro ring;
R¹ is H, optionally substituted C₁-C₆ alkyl, optionally substituted C₁-C₆ heteroalkyl, optionally substituted C₃—C cycloalkyl, or optionally substituted C₂-C₅ heterocycloalkyl;
L is a bond, S, O, or NR¹⁰, optionally substituted C₁-C₆ alkylene, or optionally substituted C₁-C₆ heteroalkylene;
E represents a structure of Formula (Ic).

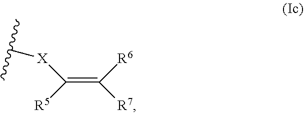

wherein,
X is C(═O), P(O)OR, C(═S), or S(═O)ₙ, where n is 1 or 2;
R² is H or optionally substituted C₁-C₃ alkyl;
R⁵ is hydrogen, cyano, halogen, C₁-C₆ alkyl, C₁-C₆ alkoxyl, C₁-C₆ heteroalkyl, C₂-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₅ heterocycloalkyl, heteroaryl, —C₁-C₆alkylene-heteoraryl, —C₁-C₆alkylene-NH(C₁-C₆ alkyl), or —C₁-C₆alkylene-N(C₁-C₅ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₂-C₆ cycloalkyl, C₂-C₆ heterocycloalkyl, heteroaryl, —C₁-C₆alkylene-heteoraryl, —C₁-C₆alkylene-NH(C₁-C₆ alkyl), or —C₁-C₆alkylene-N(C₁-C₅ alkyl)₂, each of the alkyl, alkylene, alkoxyl, heteroalkyl, cycloalkyl, heterocycloalkyl, or heteroaryl is optionally substituted;
R²⁸ and R²⁹ are each independently H, hydroxyl, C₁-C₆ alkyl, C₁-C₆ alkoxy, cyano, nitro, C₃-C₆ cycloalkyl, or C₂-C₅ heterocycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, -L^{a}-cycloalkyl, -L⁴-heterocycloalkyl, -L⁴-aryl, -L⁴-heteroaryl, —OR¹⁰, —SR¹⁰, —N(R¹⁰)(R^{10′}), —C(O)OR¹⁰, —OC(═O)N(R¹⁰)(R^{10′}), —N(R¹⁰)C(O)N(R¹⁰)(R^{10′}), —N(R^{10′})C(O)OR¹⁰, —N(R^{10′})S(O)₂R^{10′}, —C(O)R¹¹, —S(O)R¹¹. —OC(═O)R¹¹, —C(O)N(R¹⁰)(R^{10′}), —C(O)C(O)N(R¹⁰)(R^{10′}), —N(R^{10′})—, —S(O)₂R¹¹, —S(O)₂N(R¹⁰)(R^{10′})—, S(═O)(═NH)N(R¹⁰)(R^{10′}), —CH₂C(O)N(R¹⁰)(R^{10′}), —CH₂N(R¹⁰)C(O)R¹¹, —CH₂S(O)₂R¹¹, or —CH₂S(O)₂N(R¹⁰)(R^{10′}), wherein each of the alkyl, heteroalkyl, cycloalkyl, heterocycloalkyl, aryl, and heteroaryl is optionally substituted;
R³³ is H, C₁-C₆ alkyl, C₁-C₆ heteroalkyl, or C₃-C₆ cycloalkyl, wherein each of the alkyl, heteroalkyl, and cycloalkyl is optionally substituted;
each R¹⁰ is independently selected from hydrogen, C₁-C₆ alkyl, C₁-C₆ haloalkyl, C₂-C₆ alkenyl, C₂-C₆ alkynyl, C₃-C₆ cycloalkyl, C₂ heterocycloalkyl, C₆-C₁₀ aryl, and C₁-C₉heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₆heteroaryl;
each R^{10′} is independently selected from hydrogen, C₁-C₆ alkyl, and C₁-C₆ haloalkyl; and
each R¹¹ is independently selected C₁-C₆ alkyl, C₂-C₆alkenyl, C₂-C₆ alkynyl, C₃-C₆cycloalkyl, C₂-C₉ heterocycloalkyl, C₆-C₁₀ aryl, and C₁-C₉ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₁-C₄ heterocycloalkyl, C₆-C₁₀ aryl, and C₁-C₉heteroaryl;
(b) a covalently bonded radioisotope R*; and
(c) a linker covalently connecting the radioisotope R* and the structure of Formula (IV) or a salt or solvate thereof.

Embodiment 323: The radiolabeled compound of embodiment 322, or a salt or solvate thereof, wherein the radiolabeled compound comprises a structure of Formula (IVd)

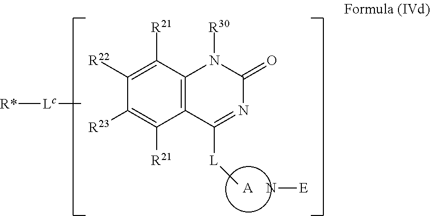

wherein
L^{C} is a linker comprising 1 to 20 groups independently selected from —CR^{b}R^{b}—, —C(═O)—, —S(═O)—. —S═O₂—, —NR^{a}—

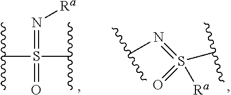

—CR^{b}═CR^{b}—, —C≡C—, —O—, —S—, —C(═O)O—, —OC═O—. —C(═O)NR^{a}—, —NR—C(═O), —S(O)₂NR^{a}—, —NR^{a}S(═O)₂—, —NR^{a}C(═O)NR^{a}—, —NR^{a}C(═O)O—, —OC(═O)NR^{a}—, arylene, heteroarylene;
each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₉heterocycloalkyl, aryl, or heteroaryl;
each R^{b} is independently hydrogen, halogen, —CN, —NO₂, —OR^{a}, —SR^{a}, C₁-C₆alkyl, C₁-C₆-haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
R²² is C₁-C₆ alkyl, C₂-C₆ alkenyl, C₂-C₆ alkynyl, cycloalkyl, —C₁-C₃alkylene-cycloalkyl, heterocycloalkyl, —C₁-C₆ alkylene-heterocycloalkyl, aryl, —C₁-C₃alkylene-aryl, heteroaryl, or —C₁-C₃alkylene-heteroaryl, wherein each of the alkyl, alkylene, alkenyl, alkynyl, cycloalkyl, heterocycloalkyl, aryl, and heteroaryl is optionally substituted;
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At); and
E, ring A, L, R²¹. R²³, and R³⁰ have the meaning defined above in Formula (IV).

Embodiment 324: The radiolabeled compound of embodiment 323, or a salt or solvate thereof, wherein the radiolabeled compound comprises a structure of Formula (IVe).

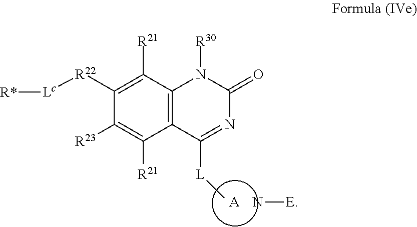

Embodiment 325: The radiolabeled compound of embodiment 323 or 324, wherein L^{C} comprises

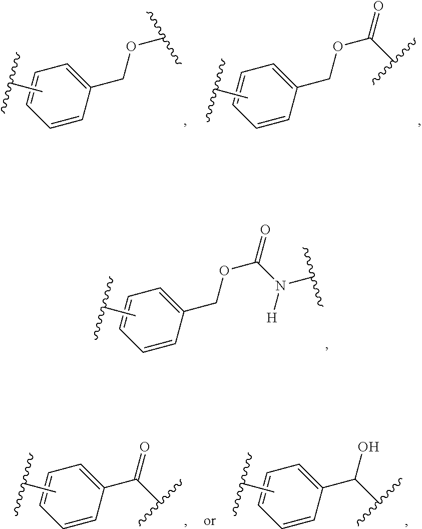

Embodiment 326: The KRAS protein of embodiment 323 or 324, wherein L^{C} comprises

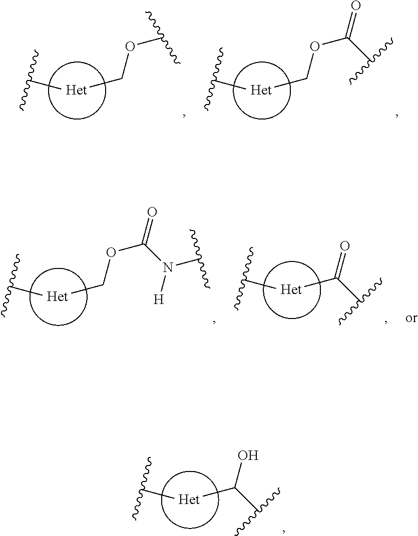

wherein Het is a 5-6 membered heteroaryl ring containing 1-2 heteroatoms independently selected from N, S, and O

Embodiment 327: The radiolabeled compound of any one of embodiments 315 to 326, wherein R* is iodine-131 (¹³¹I) or astatine-211 (²¹¹At).

Embodiment 328: The radiolabeled compound of any one of embodiments 315 to 327, wherein the radiolabeled compound is selected from Table 3A. Table 3B. Table 3C, or Table 3D.

Embodiment 329: A pharmaceutical composition comprising a radiolabeled compound of any one of embodiments 315 to 328, and a pharmaceutically acceptable excipient or carrier.

Embodiment 330: The pharmaceutical composition of embodiment 329, wherein the pharmaceutical composition is formulated for intravenous administration.

Embodiment 331: A method of making a covalently modified KRAS protein in vivo, comprising administering a radiolabeled compound of any one of embodiments 315 to 328 or a pharmaceutical composition of embodiment 329 or 330 to a subject, wherein the subject has a KRAS protein comprising a glycine to cysteine amino acid substitution at residue 12.

Embodiment 332: The method of embodiment 331, wherein the subject has cancer.

Embodiment 333: A method of treating cancer in a subject in need thereof, comprising administering to the subject a radiolabeled compound of any one of embodiments 315 to 328 or a pharmaceutical composition of embodiment 329 or 330.

Embodiment 334: The method of embodiment 332 or 333, wherein the cancer is KRAS G12C-associated cancer.

Embodiment 335: The method of any one of embodiments 332 to 334, wherein the cancer is selected from the group consisting of Cardiac cancer: sarcoma (angiosarcoma, fibrosarcoma, rhabdomyosarcoma, liposarcoma)·myxoma, rhabdomyoma, fibroma, lipoma and teratoma; Lung cancer: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma: Gastrointestinal cancer: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract cancer: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver cancer: hepatoma (hepatocellular carcinoma), cholangiocarcinoma, hepatoblastoma, angiosarcoma, hepatocellular adenoma, hemangioma: Biliary tract cancer: gall bladder carcinoma, ampullary carcinoma, cholangiocarcinoma: Bone cancer: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors: Nervous system cancer: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological cancer: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic cancer: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin cancer: malignant melanoma, basal cell carcinoma, squamous cell carcinoma, Kaposi's sarcoma, moles dysplastic nevi, lipoma, angioma, dermatofibroma, keloids, psoriasis; and Adrenal glands cancer: neuroblastoma.

Embodiment 336: The method of any one of embodiments 332 to 334, wherein the cancer is non-small cell lung cancer.

Embodiment 337: A method of killing a cell harboring a G12C KRAS mutation in a subject, the method comprising making a covalently modified KRAS protein of any one of embodiments 142 to 314 in the subject.

Embodiment 338: A method of killing a cell harboring a G12C KRAS mutation, the method comprising contacting a cell harboring a G12C KRAS mutation with a radiolabeled compound of any one of embodiments 315 to 328 or a pharmaceutical composition of embodiment 329 or 330, thereby delivering a dose of radiation to the cell.

Embodiment 339: A method of delivering a radionuclide to a cell comprising administering a radiolabeled compound of any one of embodiments 315 to 328 or a pharmaceutical composition of embodiment 329 or 330.

Embodiment 340: The method of embodiment 339, wherein the radiolabeled compound irreversibly binds to an intracellular protein of the cell.

Embodiment 341: A method of diagnosing cancer patients harboring a G12C KRAS mutation comprising administering to a patient a radiolabeled compound of any one of embodiments 315 to 328 or a pharmaceutical composition of embodiment 329 or 330.

Embodiment 342: A method of imaging a cancer harboring a G12C KRAS mutation comprising administering to a patient a radiolabeled compound of any one of embodiments 315 to 328 or a pharmaceutical composition of embodiment 329 or 330.

Embodiment 343: The method of embodiment 341 or 342, further comprising measuring the concentration of the radiolabeled compound accumulated in the patient.

Embodiment 344: The method of any one of embodiments 341 to 343, further comprising measuring the amount of radiation emitted from the radionuclide.

Embodiment 345: The method of any one of embodiments 341 to 344, further comprising analyzing the elimination profile of the radiolabeled compound in the patient.

Embodiment 346: The method of any one of embodiments 341 to 345, further comprising measuring an elimination half-life of the radiolabeled compound in the patient.

Embodiment 347: A method of producing a compound having a structure of Formula (VIa). Formula (VIb), Formula (VIc), or Formula (VId) in vivo, comprising administering a radiolabeled compound of any one of embodiments 316 to 319 or 322 to 325 to a subject,

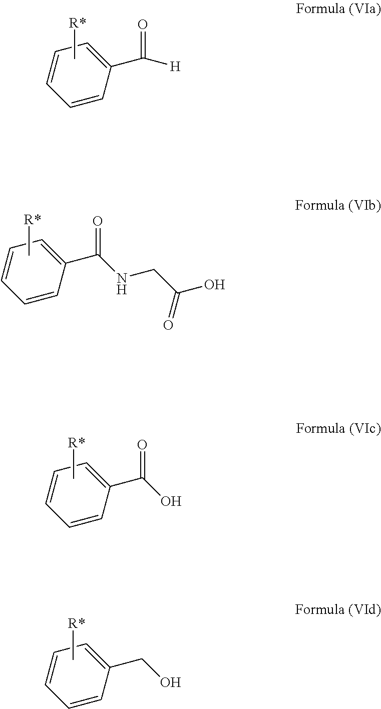

wherein
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At).

Embodiment 348: A method of excreting a compound having a structure of Formula (VIa). Formula (VIb), Formula (VIc), or Formula (VId) from a subject's body, comprising administering a radiolabeled compound of any one of any one of embodiments 316 to 319 or 322 to 325 to a subject.

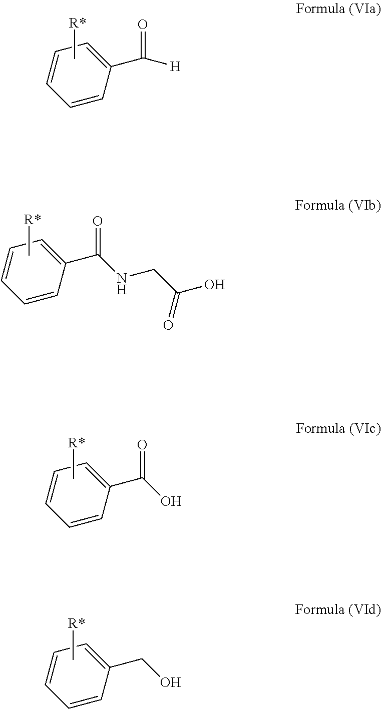

wherein
R* is fluorine-18 (¹⁸F), iodine-131 (¹³¹I), iodine-123 (¹²³I), iodine-124 (¹²⁴I), iodine-125(¹²⁵I), or astatine-211 (²¹¹At).

Embodiment 349: The method of embodiment 347 or 348, wherein the compound having a structure of Formula (VIa) is

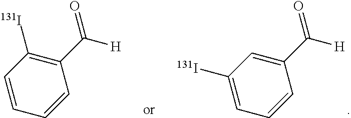 [SMILES: O=Cc1ccccc1[131I] ; O=Cc1cccc([131I])c1]

Embodiment 350: The method of embodiment 347 or 348, wherein the compound having a structure of Formula (VIb) is

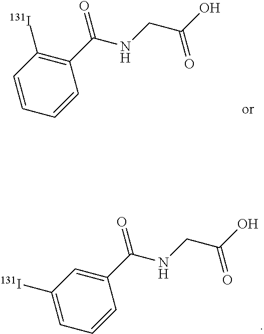 [SMILES: O=C(O)CNC(=O)c1ccccc1[131I] ; O=C(O)CNC(=O)c1cccc([131I])c1]

Embodiment 351: The method of embodiment 347 or 348, wherein the compound having a structure of Formula (VIc) is

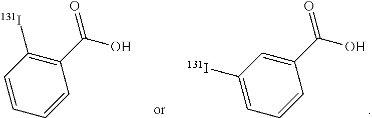 [SMILES: O=C(O)c1ccccc1[131I] ; O=C(O)c1cccc([131I])c1]

Embodiment 352: The method of embodiment 347 or structure of Formula (VId) is

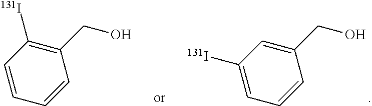 [SMILES: OCc1ccccc1[131I] ; OCc1cccc([131I])c1]

We claim:

1. A radiopharmaceutical conjugate comprising:
(a) a targeting ligand that covalently binds to an intracellular KRAS protein, wherein the intracellular KRAS protein is mutated, and wherein the targeting ligand comprises a structure of Formula (III), or a salt or solvate thereof, 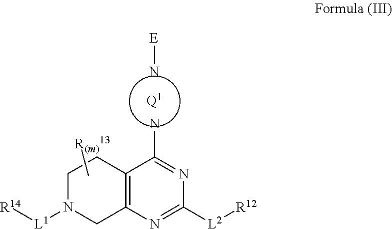
wherein
ring Q¹ is a 4-12 membered saturated or partially saturated monocyclic or bicyclic ring, wherein the monocyclic or bicyclic ring is optionally substituted;
L¹ is a bond, —C(═O)—, or optionally substituted C₁-C₃ alkylene;
L² is a bond, —C(═O)—, O, S or NR¹⁵,
E is 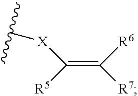
X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₅ heterocycloalkyl, wherein each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₇ heterocycloalkyl, or heteroaryl, wherein each of the alkyl, alkoxyl, heteroalkyl, cycloalkyl, heterocycloalkyl, or heteroaryl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₂-C₆ heterocycloalkyl, or heteroaryl, wherein each of the alkyl, alkoxyl, heteroalkyl, cycloalkyl, heterocycloalkyl, or heteroaryl is optionally substituted;
R¹² is hydrogen, C₁-C₆ alkyl, C₁-C₆ heteroalkyl, -L³—NR¹⁵R^{15′}, heterocycloalkyl, -L³-heterocycloalkyl, cycloalkyl, -L³-cycloalkyl, aryl, heteroaryl, -L³-aryl, or -L³-heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted;
each R¹³ is independently OH, halogen, oxo, substituted or unsubstituted C₁-C₆ alkyl, or substituted or unsubstituted C₁-C₆ heteroalkyl,
R¹⁴ is hydrogen, cycloalkyl, heterocycloalkyl, aryl, or heteroaryl, wherein each of the cycloalkyl, heterocycloalkyl, aryl, and heteroaryl is optionally substituted;
each R¹⁵ is independently hydrogen or C₁-C₃ alkyl;
each R^{15′} is independently hydrogen, acyl, C₁-C₃ alkyl, C₁-C₃ heteroalkyl or C₁-C₃ hydroxyalkyl;
m is 0, 1, or 2,
wherein the structure of Formula (III) is attached to the rest of the conjugate at any suitable position; and
(b) a radionuclide.

2. The radiopharmaceutical conjugate of claim 1, wherein
ring Q¹ is a 4-12 membered saturated or partially saturated monocyclic or bicyclic ring optionally substituted with one, two, or three groups selected from R¹⁸;
L¹ is a bond, —C(═O)—, or optionally substituted C₁-C₃ alkylene, wherein the alkylene is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅heterocycloalkyl;
L² is a bond, —C(═O)—, O, S or NR¹⁵;
E is 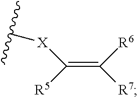
X is C(═O), P(═O)OR², C(═S), or S(═O)ₙ, where n is 1 or 2;
R² is hydrogen or substituted or unsubstituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₆ heterocycloalkyl;
R⁵ is hydrogen, cyano, halogen, C₁-C₆ alkyl, C₁-C₆ alkoxyl, C₁-C₆ heteroalkyl, C₂-C₆ cycloalkyl, or C₂-C₆ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅ heterocycloalkyl;
R⁷ is hydrogen, cyano, halogen, C₁-C₆ alkyl, C₁-C₆ alkoxyl, C₁-C₆ heteroalkyl, C₃-C₆ cycloalkyl, C₂-C₇ heterocycloalkyl, or C₁-C₉heteroaryl, wherein each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅heterocycloalkyl; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyano, halogen, C₁-C₆ alkyl, C₁-C₆ alkoxyl, C₁-C₆ heteroalkyl, C₃-C₆ cycloalkyl, C₂-C₆ heterocycloalkyl, or C₁-C₆heteroaryl, wherein, each of the alkyl, alkoxyl, heteroalkyl, cycloalkyl, heterocycloalkyl, or heteroaryl is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, and C₂-C₅ heterocycloalkyl;
R¹² is hydrogen, C₁-C₆ alkyl, C₁-C₆ heteroalkyl, -L³—NR¹⁵R^{15′}, C₂-C₁₂heterocycloalkyl, -L³-C₂-C₁₂heterocycloalkyl, C₃-C₁₅cycloalkyl, -L³-C₃-C₁₅cycloalkyl, aryl, C₁-C₉heteroaryl, -L³-aryl, or -L³-C₁-C₉heteroaryl, wherein each of the heterocycloalkyl, cycloalkyl, aryl, heteroaryl, alkyl or heteroalkyl is optionally substituted with one, two, three or four groups selected from R¹⁹;
L³ is C₁-C₄ alkylene or C₁-C₄ heteroalkylene, each of which is optionally substituted with one, two, or three groups selected from R¹⁹;
each R¹³ is independently —OH, halogen, oxo, substituted or unsubstituted C₁-C₆ alkyl, or substituted or unsubstituted C₁-C₆ heteroalkyl, wherein each of the alkyl and heteroalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅heterocycloalkyl;
R¹⁴ is hydrogen, C₃-C₁₅cycloalkyl, C₂-C₁₂heterocycloalkyl, aryl, or C₁-C₉heteroaryl, wherein each of the cycloalkyl, heterocycloalkyl, aryl, and heteroaryl is optionally substituted with one, two, three or four groups selected from R¹⁶;
each R¹⁵ is independently hydrogen or C₁-C₃ alkyl;
each R^{15′} is independently hydrogen, acyl, C₁-C₃ alkyl, C₁-C₃ heteroalkyl or C₁-C₃ hydroxyalkyl;
m is 0, 1, or 2;
each R¹⁷ is independently halogen, hydroxyl, C₁-C₆ alkyl, C₃-C₆ cycloalkyl, C₁-C₆ alkoxy, C₁-C₆ haloalkyl, amino, cyano, C₁-C₆ heteroalkyl, C₁-C₆ hydroxyalkyl, —O—C₁-C₆haloalkyl, or —S—C₁-C₆haloalkyl;
each R¹⁸ is independently halogen, oxo, C₁-C₆ alkyl, C₁-C₆ aminoalkyl, C₁-C₆haloalkyl, C₂-C₆ alkenyl, C₂-C₆ alkynyl, C₁-C₆ heteroalkyl, —CN, —C(O)OR¹⁵, —C(O)N(R¹⁵)(R^{15′}), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, and C₂-C₅ heterocycloalkyl;
R¹⁶ and R¹⁹ are each independently selected from halogen, oxo, —CN, C₁-C₆ alkyl, C₂-C₆ alkenyl, C₂-C₆ alkynyl, C₁-C₆ heteroalkyl, C₃-C₁₀cycloalkyl, —C₁₋₃alkylene-C₃₋₆cycloalkyl, C₂-C₉ heterocycloalkyl, —C₁₋₃alkylene-C₂-9heterocycloalkyl, C₆-C₁₀aryl, —C₁₋₃alkylene-C₃₋₆aryl, C₁-C₉ heteroaryl, —C₁₋₃alkylene-C₁₋₉heteroaryl, —OR¹⁰, —SR¹⁰, —N(R¹⁰)(R^{10′}), —C(O)OR, —OC(═O)N(R¹⁰)(R^{10′}), —N(R^{10′})C(═O)N(R¹⁰)(R^{10′}), —N(R^{10′})C(═O)OR^{a}, —N(R¹⁰)S(═O)₂R¹¹, —C(═O)R¹¹, —S(C)R¹¹, —OC(═O)R¹¹, —C(═O)N(R¹⁰)(R^{10′}), —C(═O)C(═O)N(R¹⁰)(R^{10′}), —N(R¹⁰)C(O)R¹¹, —S(O)₂R¹¹, —S(O)₂N(R¹⁰)(R^{10′}), —S(═O)(═NH)N(R¹⁰)(R^{10′}), —CH₂C(O)N(R¹⁰)(R^{10′}), —CH₂N(R^{10′})C(═O)R¹¹, —CH₂S(═O)₂R¹¹, and —CH₂S(═O)₂N(R)(R¹⁰), wherein the alkyl, alkenyl, alkynyl, cycloalkyl, —C₁₋₃alkylene-C₃₋₆cycloalkyl, heterocycloalkyl, C₁₋₃alkylene-C₁-C₉heterocycloalkyl, aryl, —C₁₋₃alkylene-C₆₋₁₀aryl, heteroaryl and —C₁₋₃alkylene-C₁₋₉ heteroaryl are optionally substituted with one, two, three, or four groups independently selected from halogen, oxo, —CN, C₁-C₆ alkyl, C₁-C₆ haloalkyl, C₁-C₆ alkoxy, C₁-C₆ alkyloxy, C₁-C₆haloalkoxy, C₃-C₁₀cycloalkyl, C₂-C₉heterocycloalkyl, C₆-C₁₀aryl, C₁-C₉heteroaryl, —OR¹⁰, —SR¹⁰, —N(R¹⁰)(R^{10′}), —C(═O)OR¹⁰, —OC(═O)N(R¹⁰)(R^{10′}), —N(R^{10′})C(═O)N(R¹⁰)(R^{10′}), —N(R^{10′})C(═O)OR^{a}, —N(R¹⁰)S(═O)₂R¹⁰, —C(═O)R¹⁰, —S(O)R¹¹, —OC(═O)R¹⁰, —C(═O)N(R¹⁰)(R^{10′}), —C(O)C(═O)N(R¹⁰)(R^{10′}), —N(R¹⁰)C(═O)R¹⁰, —S(═O)₂R¹⁰, —S(═O)₂N(R¹⁰)(R^{10′})—, S(═O)(═NH)N(R¹⁰)(R^{10′}), —CH₂C(═O)N(R¹⁰)(R^{10′}), —CH₂N(R¹⁰)C(═O)R¹¹, —CH₂S(═O)₂R¹¹, and —CH₂S(═O)₂N(R)(R¹⁰);
each R¹⁰ is independently selected from hydrogen, C₁-C₆ alkyl, C₁-C₆ haloalkyl, C₂-C₆ alkenyl, C₂-C₆ alkynyl, C₃-C₆ cycloalkyl, C₂-₉heterocycloalkyl, C₆-C₁₀ aryl, and C₁-C₉heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, C₂-C₉ heterocycloalkyl, C₆-C₁₀ aryl, and C₁-C₄ heteroaryl;
each R^{10′} is independently selected from hydrogen, C₁-C₆ alkyl, and C₁-C₆ haloalkyl; and
each R¹¹ is independently selected C₁-C₆ alkyl, C₂-C₆ alkenyl, C₂-C₆ alkynyl, C₃-C₆ cycloalkyl, C₂-C₉ heterocycloalkyl, C₆-C₁₀ aryl, and C₁-C₉heteroaryl, wherein the alkyl, alkenyl, alkynl, cycloalkyl, heterocycloalkyl, aryl, and heteroaryl are optionally substituted with one, two, or three groups selected from halogen, —CN, hydroxy, C₁-C₆ alkyl, C₁-C₆, haloalkyl, C₁-C₆ alkoxy, C₃-C₆ cycloalkyl, C₂-C₉ heterocycloalkyl, C₆-C₁, aryl, and C₁-C₉heteroaryl.

3. The radiopharmaceutical conjugate of claim 1 or claim 2, wherein
R¹² is C₃-C₁₅cycloalkyl, C₂-C₁₂heterocycloalkyl, -L³-C₂-C₁₂heterocycloalkyl, or -L³-C₃-C₁₅cycloalkyl, wherein each of the L³, heterocycloalkyl, cycloalkyl, alkyl, or heteroalkyl is optionally substituted with one, two, three or four groups selected from R¹⁹; and
wherein each R¹⁹ is independently selected from oxo, —CN, C₁-C₆ alkyl, C₁-C₆ heteroalkyl, C₃-C₁₀ cycloalkyl, —C₁₋₃alkylene-C₃₋₆cycloalkyl, C₂-C₉ heterocycloalkyl, —C₁₋₃alkylene-C₂₋₉heterocycloalkyl, C₆-C₁₀ aryl, —C₁₋₃alkylene-C₆₋₁₀aryl, C₁₋₉ heteroaryl, —C₁₋₃alkylene-C₁₋₉ heteroaryl, —OR¹⁰, —SR¹⁰, —N(R¹⁰)(R^{10′}), —C(O)OR¹⁰, —O C(═O)N(R¹⁰)(R^{10′}), —N(R¹⁰)C(═O)N(R¹⁰)(R^{10′}), —N(R¹⁰)C(═O)OR^{a}, —N(R¹⁰)S(═O)₂R¹¹, —C(═O)R¹¹, —S(═O)R¹¹, —OC(═O)R¹¹, —C(═O)N(R¹⁰)(R^{10′}), —C(═O)C(═O)N(R¹⁰)(R^{10′}), —N(R^{10′})C(O)R¹¹, —S(O)₂R¹¹, —S(O)₂N(R¹⁰)(R^{10′}), —S(═O)(═NH)N(R¹⁰)(R^{10′}), —CH₂C(O)N(R¹⁰)(R^{10′}), —CH₂N(R¹⁰)C(═O)R¹¹, —CH₂S(═O)₂R¹¹, and —CH₂S(═O)₂N(R¹⁰)(R^{10′}), wherein the alkyl, alkenyl, alkynyl, cycloalkyl, —C₁₋₃alkylene-C₃₋₆cycloalkyl, heterocycloalkyl, —C₁₋₃alkylene-C₂-C₉heterocycloalkyl, aryl, —C₁₋₃alkylene-C₆₋₁₀aryl, heteroaryl and —C₁₋₃alkylene-C₁₋₉heteroaryl are optionally substituted with one, two, three, or four groups independently selected from halogen, oxo, —CN, C₁-C₆ alkyl, C₁-C₆ haloalkyl, C₁-C₆ alkoxy, C₁-C₆ alkyloxy, C₁-C₆ haloalkoxy, C₃-C₁₀cycloalkyl, C₂-C₉ heterocycloalkyl, C₆-C₁₀aryl, C₁-C₄heteroaryl, OR¹⁰, —SR¹⁰, —N(R¹⁰)(R^{10′}), —C(O)OR¹⁰, —OC(═)N(R¹⁰)(R^{10′}), —N(R^{10′})C(═O)N(R¹⁰)(R^{10′}), —N(R^{10′})C(═O)OR¹¹, —N(R^{10′})S(═O), R¹¹, —C(═O)R¹¹, —S(O)R¹¹, —OC(═O)R¹¹, —C(═O)N(R¹⁰)(R^{10′}), —C(═O)C(═O)N(R¹⁰)(R^{10′}), —N(R^{10′})C(═O)R¹¹, —S(═O)—R¹¹, —S(═O), N(R¹⁰)(R^{10′})—, S(═O)(═NH)N(R¹⁰)(R^{10′}), —CH₂C(═O)N(R¹⁰)(R^{10′}), —CH₂N(R^{10′})C(═O)R¹¹, —CH—S(═O)₂R¹¹, and —CH₂S(═O)₂N(R¹⁰)(R^{10′}).

4. The radiopharmaceutical conjugate of claim 3, wherein
R¹² is -L³-C₂-C₁₂heterocycloalkyl, optionally substituted with one, two, three or four groups selected from R¹⁹; and
wherein each R¹⁹ is independently selected from oxo, C₁-C₆ alkyl, C₁-C₆ heteroalkyl, —C₁₋₃alkylene-C₆₋₁₀aryl, —C₁₋₃alkylene-C₁₋₉heteroaryl, and OR¹⁰, wherein the alkyl, heteroalkyl, and —C₁₋₃alkylene-C₆₋₁₀aryl, and —C₁₋₃alkylene-C₁₋₉heteroaryl are optionally substituted with one, two, three, or four groups independently selected from C₁-C₆ alkyl, C₆-C₁₀ aryl, —OR, —C(═O)OR¹⁰, or —N(R^{10′})C(═O)R¹¹.

5. The radiopharmaceutical conjugate of claim 4, wherein, the targeting ligand comprises a structure of Formula (IIIa-1) or Formula (IIIa-2), or a salt or solvate thereof,

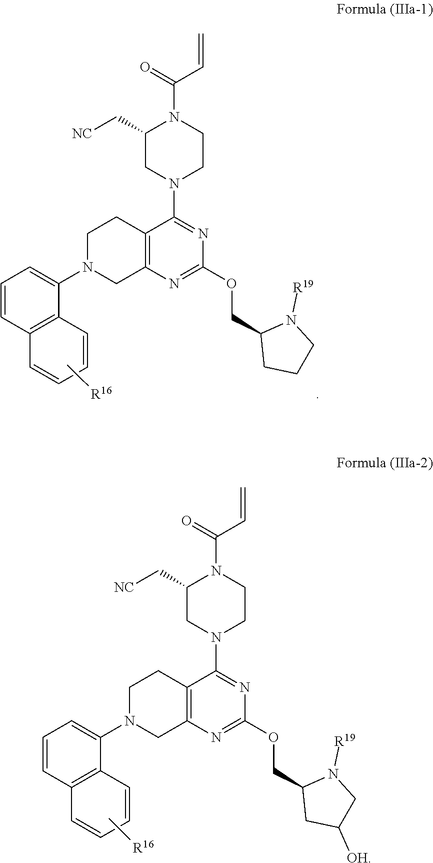

6. The radiopharmaceutical conjugate of any one of claims 1-5, wherein the structure of Formula (III) is attached to the rest of the conjugate through R¹⁹.

7. The radiopharmaceutical conjugate of any one of claims 1-6, wherein
R¹⁹ is 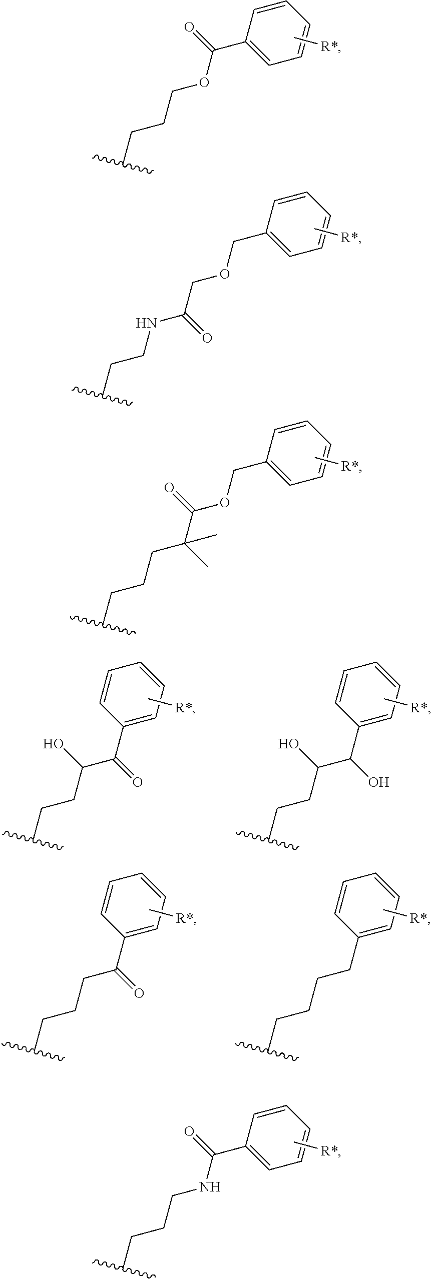 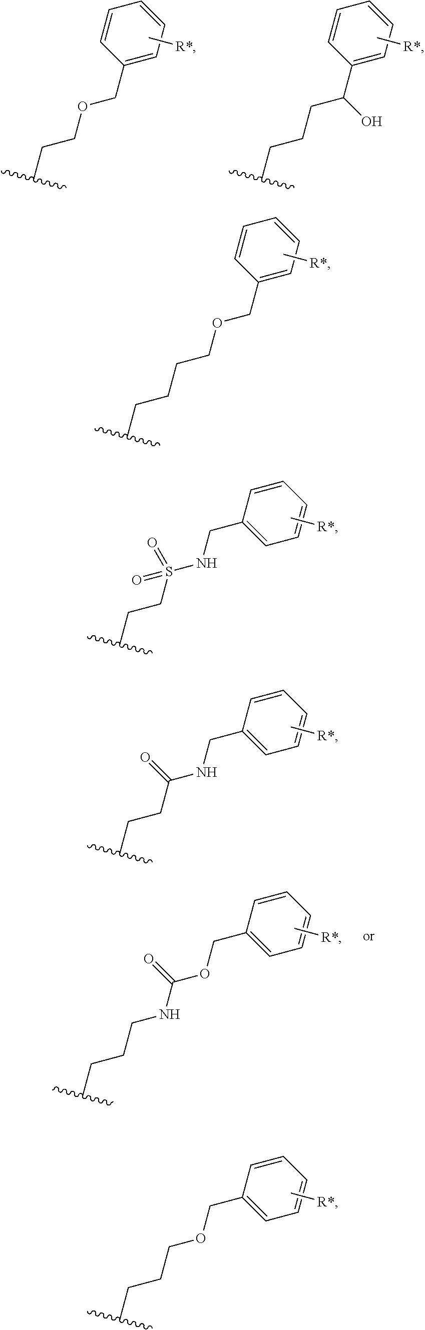
wherein R* is the radionuclide.

8. The radiopharmaceutical conjugate of any one of claims 1-5, wherein
R¹⁹ is

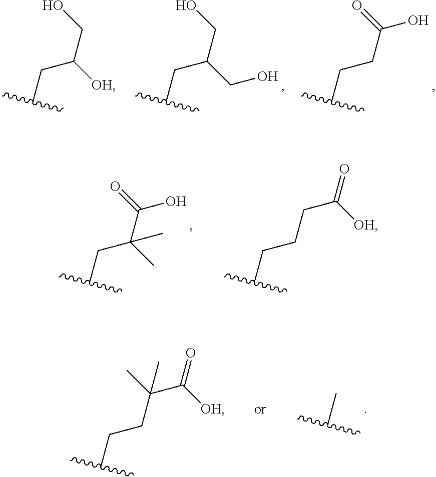

9. The radiopharmaceutical conjugate of any one of claims 1-4 or 6-8, wherein;
R⁵ is hydrogen, halogen or a C₁-C₃ alkyl optionally substituted with one to three substituents selected from hydroxyl and halogen;
R⁶ is hydrogen, cyano, halogen, optionally substituted C₂-C₅ alkyl, optionally substituted C₁-C₆ alkoxyl, optionally substituted C₁-C₆ heteroalkyl, optionally substituted C₃-C₆ cycloalkyl, or optionally substituted C₂-C₆ heterocycloalkyl; and
R⁷ is hydrogen, cyano, halogen, optionally substituted C₁-C₆ alkyl, optionally substituted C₁-C₆ alkoxyl, or optionally substituted C₁-C₆ heteroalkyl.

10. The radiopharmaceutical conjugate of any one of claims 1-4 or 6-9, wherein:
E is

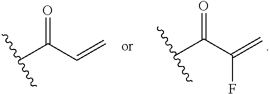

11. The radiopharmaceutical conjugate of any one of claims 1-4 or 6-10, wherein
ring Q¹ is a 6 membered monocyclic ring optionally substituted with one to three R¹⁸, wherein R¹⁸ is methyl, —CH₂CN, oxo, hydroxyl, carboxyl, C(O)OR¹⁵.

12. The radiopharmaceutical conjugate of any one of claims 1-4 or 6-11, wherein the targeting ligand comprises a structure of Formula (IIIa), or a salt or solvate thereof, 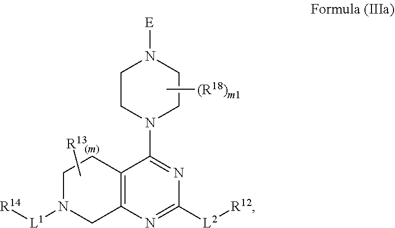
wherein
each R¹¹ is independently halogen, oxo, C₁-C₆ alkyl, C₁-C₆ aminoalkyl, C₁-C₃: haloalkyl, C₂-C₆ alkenyl, C₂-C₆ alkynyl, C₁-C₆ heteroalkyl, —CN, —C(O)OR¹⁵, —C(O)N(R¹⁵)(R¹⁵), —N(R¹⁵)(R^{15′}), or OR¹⁵, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C)—Co alkyl, C₁-C₆ haloalkyl, C₁-C₆ alkoxy, C₁-C₆cycloalkyl, and C₂-C₅ heterocycloalkyl; and
m1 is 0, 1, 2, or 3.

13. The radiopharmaceutical conjugate of any one of claims 1-4 or 6-12, wherein
R¹⁴ is phenyl, naphthyl, or monocyclic or bicyclic heteroaryl, each optionally substituted with one or more R¹⁶,
wherein each R¹⁶ is independently halogen, —CN, —N(R¹⁰)(R^{10′}), —OR¹⁰, oxo, —C(O)OR¹⁰, —C(O)N(R¹⁰)(R^{10′}), —SR¹⁰, —OC(═O)R¹¹, —S(O)R¹¹, S(O)₂R¹¹, —S(O)₂N(R¹⁰)(R^{10′}), C₁-C₆ alkyl, C₃-C₆ cycloalkyl, C₁-C₆ fluoroalkyl, C₁-C₆heteroalkyl, C₁-C₆alkoxy, C₁-C₆ fluoroalkoxy, C₂-C₆ heterocycloalkyl, C₆-C₁₀aryl, or C₁-C₉heteroaryl, wherein each of the alkyl, cycloalkyl, fluoroalkyl, heteroalkyl, alkoxy, fluoroalkoxy, heterocycloalkyl, aryl, and heteroaryl are optionally substituted with one, two, three, or four groups independently selected from halogen, oxo, —CN, C₁-C₆ alkyl, C₂-C₅ haloalkyl, C₁-C₆ alkoxy, C₁-C₆ alkyloxy, C₁-C₆ haloalkoxy, C₃-C₁₀ cycloalkyl, C₂-C₉heterocycloalkyl, C₆-C₁₀aryl, C₁-C₉ heteroaryl, —OR¹⁰, —SR¹⁰, —N(R¹⁰)(R^{10′})—C(O)OR¹⁰, —OC(═O)N(R¹⁰)(R^{10′}), —N(R^{10′})C(O)N(R¹⁰)(R^{10′}), —N(R^{10′})C(O)OR¹⁰, —N(R^{10′})S(O)₂R¹¹, —C(O)R¹¹, —S(O)R¹¹, —OC(═O)R¹¹, —C(O)N(R¹⁰)(R^{10′}), —C(O)C(O)N(R¹⁰)(R^{10′}), —N(R¹⁰)C(O)R¹¹, —S(O)₂R¹¹, —S(O)₂N(R¹⁰)(R^{10′})—, S(O)(═NH)N(R¹⁰)(R^{10′}), —CH—C(O)N(R¹⁰)(R^{10′}), —CH₂N(R^{10′})C(O)R¹¹, —CH₂S(O)₂R¹¹, and —CH₂S(O)₂N(R¹⁰)(R^{10′}).

14. The radiopharmaceutical conjugate of any one of claims 1-13, wherein
each R¹⁶ is independently oxo, halogen, —CN, —NH₂, —NH(CH₃), —N(CH₂)₂, —OH, —CO₂H, —C(═O)OC₁-C₄alkyl, —OC(═O)C₁-C₄ alkyl, —C(═O)NH₂, —C(═O)NH(C₁-C₄ alkyl), —C(═O)N(C₁-C₄alkyl)₂, —S(═O)₂NH₂, —S(═O)₂NH(C₁-C₄ alkyl), —S(O)₂N(C₁-C₄alkyl)₂, C₁-C₄alkyl, C₃-C₆ cycloalkyl, C₁-C₄fluoroalkyl, C₁-C₆ heteroalkyl, C₁-C₄alkoxy, C₁-C₄fluoroalkoxy, —S—C₁₋₄alkyl, —S(═O)C₁₋₄ alkyl, or —S(═O)₂(C₁-C₄ alkyl).

15. The radiopharmaceutical conjugate of any one claims 1-4 or 6-14, wherein
R¹⁴ is naphthyl optionally substituted with one or more R¹⁶, wherein each R¹⁶ is independently halogen, hydroxyl, C₁-C₃ alkyl, alkoxy, haloalkyl, amino, or cyano.

16. The radiopharmaceutical conjugate of any one claims 1-15, wherein
R¹⁶ is halogen and the halogen is the radionuclide selected from fluorine-18 (¹⁸F), iodine-131 (¹³¹I), iodine-123 (¹²³I), iodine-124 (¹²⁴I), iodine-125 (¹²⁵I), and astatine-211 (²¹¹At).

17. The radiopharmaceutical conjugate of any one of claims 1-4 or 6-15, wherein
R¹⁴ comprises the radionuclide and the radionuclide is a covalently bonded, wherein the radionuclide is selected from fluorine-18 (¹⁸F), iodine-131 (¹³¹I), iodine-123 (¹²³I), iodine-124 (¹²⁴I), iodine-125 (¹²⁵I), and astatine-211 (²¹¹At).

18. The radiopharmaceutical conjugate of any one of claims 1-4 or 6-17, wherein
R¹⁴ is

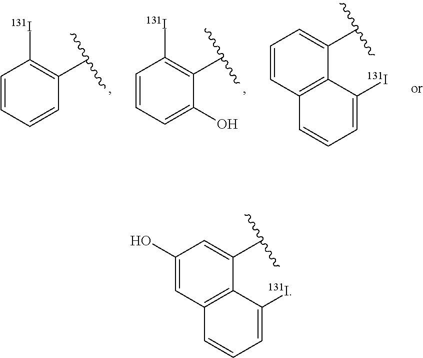

19. The radiopharmaceutical conjugate of any one of claims 1-4 or 6-18, wherein
L³ is C₁-C₄ alkylene or C₁-C₄ heteroalkylene, each of which is optionally substituted with one or more R¹⁹.

20. The radiopharmaceutical conjugate of any one of claims 1-4 or 6-19, wherein
X is C(═O);
L¹ is a bond;
L² is a bond, O, S or NR¹⁵, and
L³ is —CH₂—.

21. The radiopharmaceutical conjugate of any one of claims 1-4 or 6-20, wherein
m is 0 or 1, and
each R¹³ is independently OH, halogen, or C₁-C₃ alkyl.

22. The radiopharmaceutical conjugate of any one of claims 1-21, wherein the radiopharmaceutical conjugate has a structure of

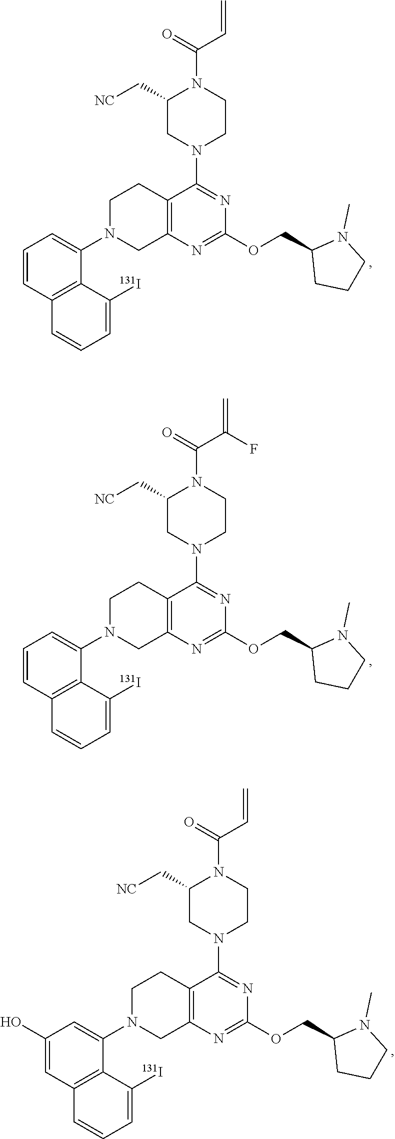 [SMILES: C=CC(=O)N1CCN(c2nc(OC[C@@H]3CCCN3C)nc3c2CCN(c2cccc4cccc([131I])c24)C3)C[C@@H]1CC#N ; C=C(F)C(=O)N1CCN(c2nc(OC[C@@H]3CCCN3C)nc3c2CCN(c2cccc4cccc([131I])c24)C3)C[C@@H]1CC#N ; C=CC(=O)N1CCN(c2nc(OC[C@@H]3CCCN3C)nc3c2CCN(c2cc(O)cc4cccc([131I])c24)C3)C[C@@H]1CC#N]

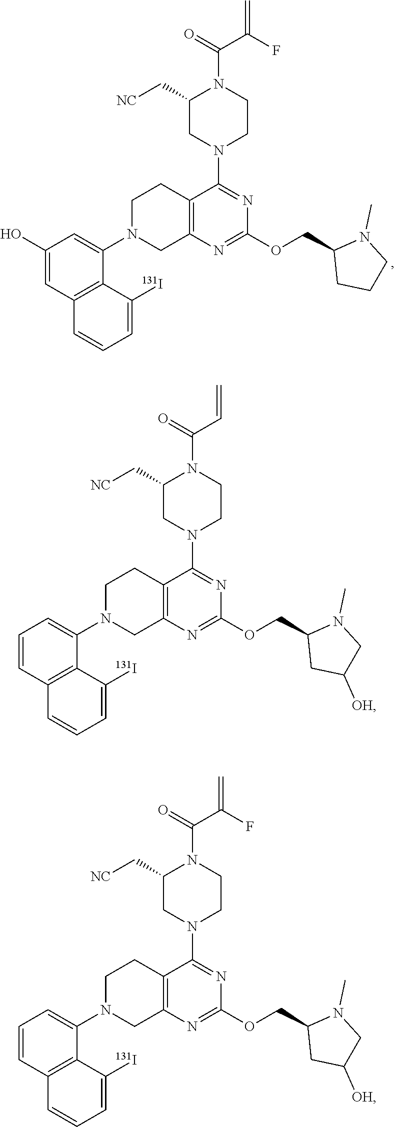 [SMILES: C=C(F)C(=O)N1CCN(c2nc(OC[C@@H]3CCCN3C)nc3c2CCN(c2cc(O)cc4cccc([131I])c24)C3)C[C@@H]1CC#N ; C=CC(=O)N1CCN(c2nc(OC[C@@H]3CC(O)CN3C)nc3c2CCN(c2cccc4cccc([131I])c24)C3)C[C@@H]1CC#N ; C=C(F)C(=O)N1CCN(c2nc(OC[C@@H]3CC(O)CN3C)nc3c2CCN(c2cccc4cccc([131I])c24)C3)C[C@@H]1CC#N]

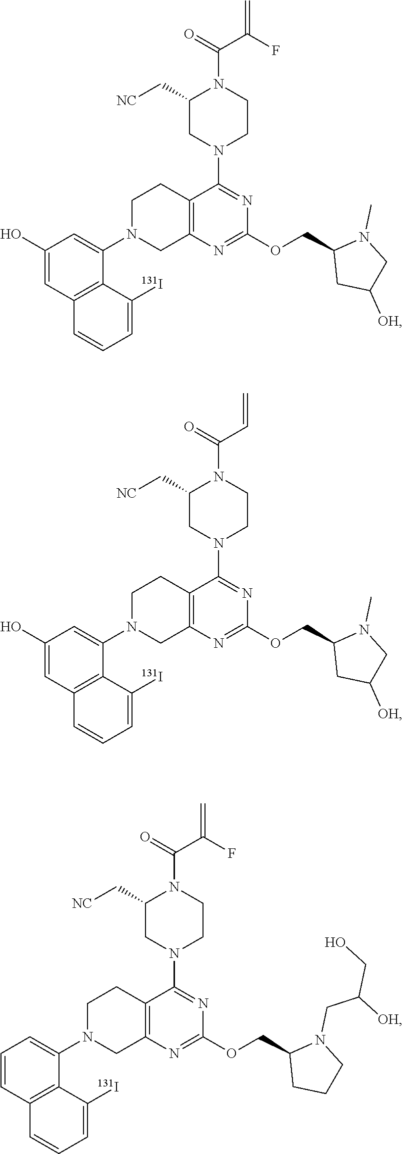 [SMILES: C=C(F)C(=O)N1CCN(c2nc(OC[C@@H]3CC(O)CN3C)nc3c2CCN(c2cc(O)cc4cccc([131I])c24)C3)C[C@@H]1CC#N ; C=CC(=O)N1CCN(c2nc(OC[C@@H]3CC(O)CN3C)nc3c2CCN(c2cc(O)cc4cccc([131I])c24)C3)C[C@@H]1CC#N ; C=C(F)C(=O)N1CCN(c2nc(OC[C@@H]3CCCN3CC(O)CO)nc3c2CCN(c2cccc4cccc([131I])c24)C3)C[C@@H]1CC#N]

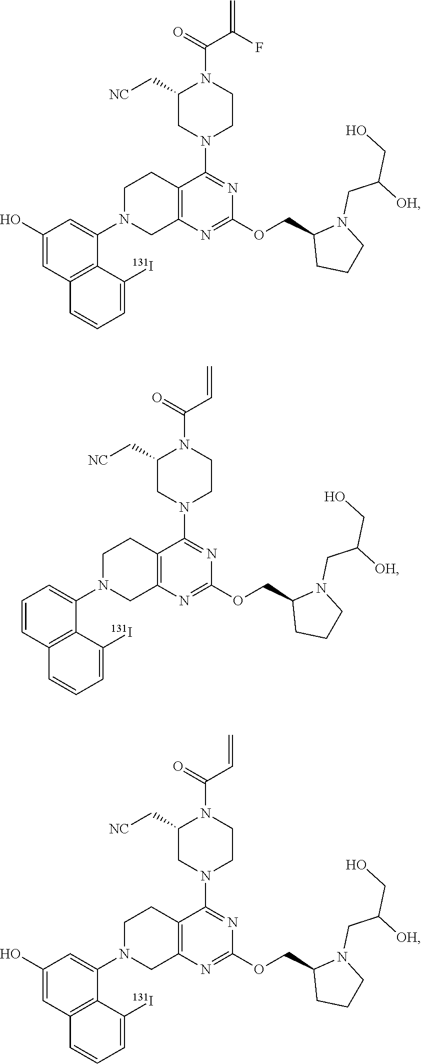 [SMILES: C=C(F)C(=O)N1CCN(c2nc(OC[C@@H]3CCCN3CC(O)CO)nc3c2CCN(c2cc(O)cc4cccc([131I])c24)C3)C[C@@H]1CC#N ; C=CC(=O)N1CCN(c2nc(OC[C@@H]3CCCN3CC(O)CO)nc3c2CCN(c2cccc4cccc([131I])c24)C3)C[C@@H]1CC#N ; C=CC(=O)N1CCN(c2nc(OC[C@@H]3CCCN3CC(O)CO)nc3c2CCN(c2cc(O)cc4cccc([131I])c24)C3)C[C@@H]1CC#N]

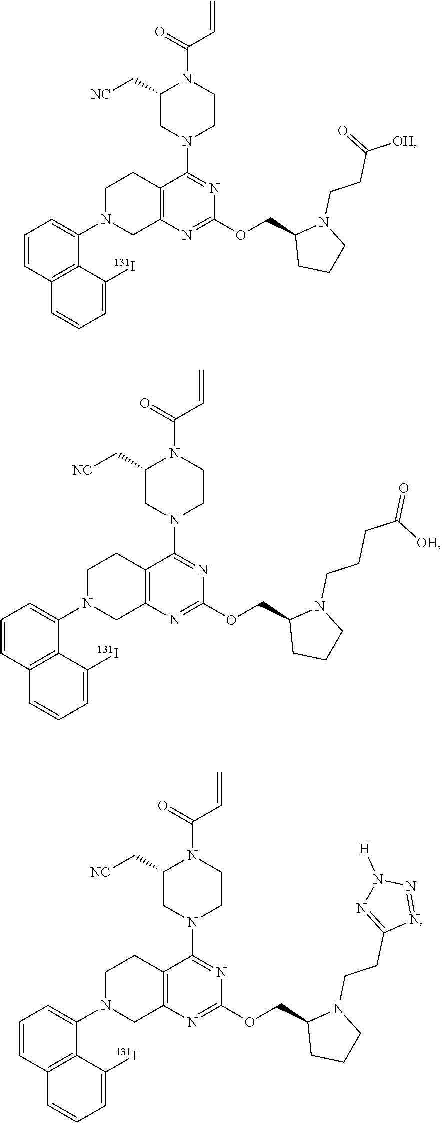 [SMILES: C=CC(=O)N1CCN(c2nc(OC[C@@H]3CCCN3CCC(=O)O)nc3c2CCN(c2cccc4cccc([131I])c24)C3)C[C@@H]1CC#N ; C=CC(=O)N1CCN(c2nc(OC[C@@H]3CCCN3CCCC(=O)O)nc3c2CCN(c2cccc4cccc([131I])c24)C3)C[C@@H]1CC#N ; C=CC(=O)N1CCN(c2nc(OC[C@@H]3CCCN3CCc3nn[nH]n3)nc3c2CCN(c2cccc4cccc([131I])c24)C3)C[C@@H]1CC#N]

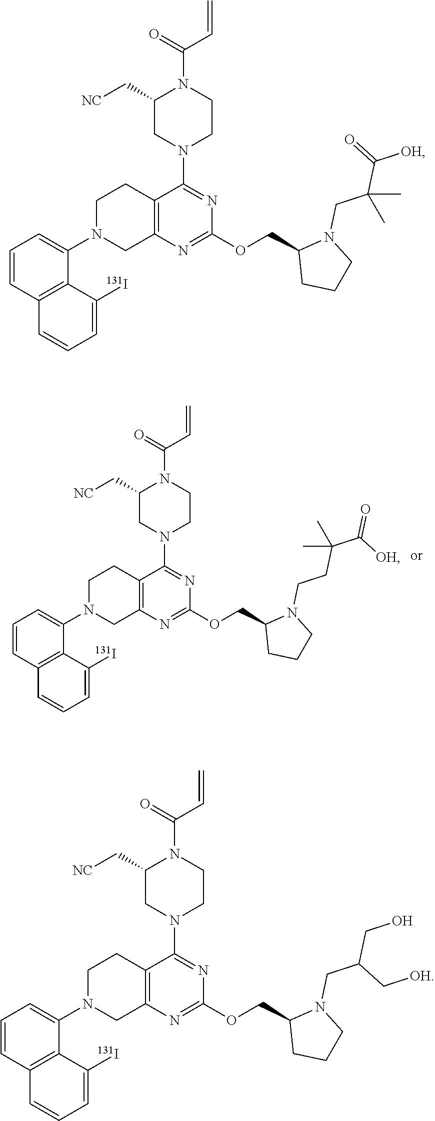 [SMILES: C=CC(=O)N1CCN(c2nc(OC[C@@H]3CCCN3CC(C)(C)C(=O)O)nc3c2CCN(c2cccc4cccc([131I])c24)C3)C[C@@H]1CC#N ; C=CC(=O)N1CCN(c2nc(OC[C@@H]3CCCN3CCC(C)(C)C(=O)O)nc3c2CCN(c2cccc4cccc([131I])c24)C3)C[C@@H]1CC#N ; "or" ; C=CC(=O)N1CCN(c2nc(OC[C@@H]3CCCN3CC(CO)CO)nc3c2CCN(c2cccc4cccc([131I])c24)C3)C[C@@H]1CC#N]

23. The radiopharmaceutical conjugate of any one of claims 1-22, wherein the targeting ligand is configured to form a covalent bond with a KRAS protein at the G12C position, wherein residue position numbering of the KRAS protein is based on SEQ ID NO: 1 or SEQ ID NO:2.

24. A radiopharmaceutical conjugate comprising:
(a) a targeting ligand that is configured to form a covalent bond with a KRAS protein at the G12C position, wherein residue position numbering of the KRAS protein is based on SEQ ID NO: 1 or SEQ ID NO: 2, comprising a structure of Formula (IV), or a salt or solvate thereof, 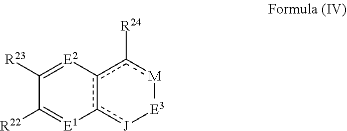
wherein
E¹ and E² are each independently N or CR²¹;
E³ is CR²⁸R²⁹, C═CR²⁸R²⁹, C═O, C═S, or C═NR²⁸;
J is N, NR³⁰, or CR³⁰;
M is N, NR³³, or CR³³, [symbol]
is a single or double bond as necessary to give every atom its normal valence;
R²¹ is independently hydrogen, hydroxyl, cyano, halogen, C₁-C₆ alkyl, C₁-C₆ alkoxyl, or C₁—C heteroalkyl, wherein each of the alkyl, alkoxy, and heteroalkyl are optionally substituted;
R²² is hydrogen, halogen, C₁-C₆ alkyl, C₂-C₆ alkenyl, C₂-C₆ alkynyl, OR^{22′}, N(R^{22′})₂, cycloalkyl, heterocycloalkyl, aryl, or heteroaryl, wherein each of the alkyl, alkylene, alkenyl, alkynyl, cycloalkyl, heterocycloalkyl, aryl, and heteroaryl is optionally substituted, and each R^{22′} is independently hydrogen, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form a 3-7-membered ring;
R²³ is hydrogen, halogen, C₁-C₆ alkyl, C₁-C₆ alkoxy, C₂-C₆ alkenyl, C₂-C₆ alkynyl, cycloalkyl, heterocycloalkyl, aryl, heteroaryl, wherein each of the alkyl, alkoxy, cycloalkyl, heterocycloalkyl, alkenyl, alkynyl, aryl, and heteroaryl is optionally substituted;
R²⁴ is 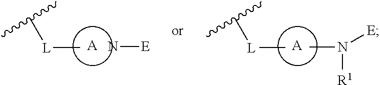
ring A is an optionally substituted 4-7 membered monocyclic ring or optionally substituted 6-11 membered bicyclic, bridged, fused, or Spiro ring;
R¹ is hydrogen, optionally substituted C₁-C₆ alkyl, optionally substituted C₁-C₆ heteroalkyl, optionally substituted C₃-C₆ cycloalkyl, or optionally substituted C₂-C₅ heterocycloalkyl;
L is a bond, C₁-C₆ alkylene, C₁-C₆ heteroalkylene, S, O, or NH;
E represents a structure of Formula (Ic), 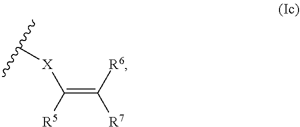
wherein,
X is C(═O), P(O)OR², C(═S), or S(═O)ₙ, where n is 1 or 2;
R² is hydrogen or optionally substituted C₁-C₃ alkyl;
R⁵ is hydrogen, cyano, halogen, C₁-C₆ alkyl, C₁-C₆ alkoxyl, C₁-C₆ heteroalkyl, C₁-C₆ cycloalkyl, or C₂-C₅ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-G, cycloalkyl, C₂-C₇ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₁-C₆ cycloalkyl, C₂-C₆ heterocycloalkyl, heteroaryl, —C₂-C₅ alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently hydrogen, hydroxyl, C₁-C₆ alkyl, C₁-C₆ alkoxy, cyano, nitro, or C₃-C₆ cycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, a yl, heteroaryl, —C₁-C₆alkylene-cycloalkyl, —C₁-C₆alkylene-heterocycloalkyl, —C₁-C₆alkylene-aryl, —C₁-C₆alkylene-heteroaryl, —C₁-C₆heteroalkylene-cycloalkyl, —C₁-C₆heteroalkylene-heterocycloalkyl, —C₁-C₆heteroalkylene-aryl, —C₁-C₆alkylene-heteroaryl wherein each of the alkyl, alkoxy, heteroalkyl, cycloalkyl, heterocycloalkyl, aryl, and heteroaryl is optionally substituted;
R³³ is hydrogen, C₁-C₆ alkyl, C₁-C₆ heteroalkyl, or C₃-C₆ cycloalkyl, wherein each of the alkyl, heteroalkyl, and cycloalkyl is optionally substituted; and
(b) a radionuclide.

25. The radiopharmaceutical conjugate of claim 24, wherein
E¹ and E² are each independently N or CR²¹;
E³ is CR²⁸R²⁹, C═CR²⁸R²⁹, C═O, C═S, or C═NR²⁸;
J is N, NR³⁰, or CR³⁰;
M is N, NR³³, or CR³³; [symbol]
is a single or double bond as necessary to give every atom its normal valence;
R²¹ is independently hydrogen, hydroxyl, cyano, halogen, C₁-C₆ alkyl, C₁-C₆ alkoxyl, or C₁-C₆ heteroalkyl, wherein each of the alkyl, alkoxy, and heteroalkyl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉ heteroaryl;
R²² is hydrogen, halogen, C₁-C₆ alkyl, C₂-C₀ alkenyl, C₂-C₆ alkynyl, OR^{22′}, N(R²²)₂, cycloalkyl, heterocycloalkyl, aryl, or heteroaryl, wherein each of the alkyl, alkylene, alkenyl, alkynyl, cycloalkyl, heterocycloalkyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₂-C₅cycloalkyl, and C₂-C₅ heterocycloalkyl, and each R² is independently hydrogen, C₁-C₆ alkyl, cycloalkyl, heterocycloalkyl, C₂-C₆ alkenyl, C₂-C₆ alkynyl, aryl, or heteroaryl, wherein each of the alkyl, cycloalkyl, heterocycloalkyl, alkenyl, alkynyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆ cycloalkyl, C₂-C₅ heterocycloalkyl, C₆-C₁₀ aryl, and C₁-C₉heteroaryl; or two R^{22′} together with the nitrogen atom to which they are attached, form an optionally substituted 3-7-membered ring with one, two, or three groups selected from halogen, —CN, —NO₂, amino, hydroxy, C₁-C₆ alkyl, C₁-C₆, haloalkyl, C₁-C₆ alkoxy, C₃-C₆cycloalkyl, C₂-C₅ heterocycloalkyl, C₆-C₁₀ aryl, and C₁-C₉heteroaryl;
R²³ is hydrogen, halogen, C₁-C₆ alkyl, C₁-C₃ alkoxy, C₂-C₆ alkenyl, C₂-C₆ alkynyl, cycloalkyl, heterocycloalkyl, aryl, heteroaryl, wherein each of the alkyl, alkoxy, cycloalkyl, heterocycloalkyl, alkenyl, alkynyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉ heteroaryl;
R²⁴ is 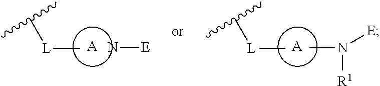
ring A is an optionally substituted 4-7 membered monocyclic ring or optionally substituted 6-11 membered bicyclic, bridged, fused, or spiro ring each of which is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉ heteroaryl;
R¹ is hydrogen, optionally substituted C₁-C₆ alkyl, optionally substituted C₁-C₆ heteroalkyl, optionally substituted C₃-C₆ cycloalkyl, or optionally substituted C₂-C₅ heterocycloalkyl each of which is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉heteroaryl;
L is a bond, C₁-C₆ alkylene, C₁-C₆ heteroalkylene, S, O, or NH;
E represents a structure of Formula (Ic), 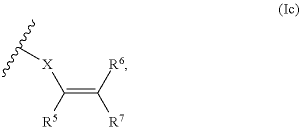
wherein,
X is C(═O), P(═O)OR², C(═S), or S(═O)ₙ, where n is 1 or 2;
R² is hydrogen or substituted or unsubstituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₆ heterocycloalkyl, C₆-C₁₀ aryl, and C₁-C₉ heteroaryl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₈ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉heteroaryl,
R⁷ is hydrogen, cyano, halogen, C₁-C₆ alkyl, C₁-C₆ alkoxyl, C₁-C₆ heteroalkyl, C₃-C₆ cycloalkyl, C₂-C₇ heterocycloalkyl, heteroaryl, —C₁-C₆alkylene-heteoraryl, —C₁-C₆alkylene-NH(C₁-C₆ alkyl), or —C₁-C₆alkylene-N(C₁-C₅ alkyl)₂, each of the alkyl, alkylene, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₇ heterocycloalkyl, C₆-C₁₀ aryl, and C₁-C₉ heteroaryl; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyano, halogen, C₁-C₆ alkyl, C₁-C₆ alkoxyl, C₁-C₆ heteroalkyl, C₃-C₆ cycloalkyl, C₂-C₆ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted with one, two, or three groups selected from halogen, —CN, hydroxy, C₁-C₆ alkyl, C₁-C₆ haloalkyl, C₁-C₆ alkoxy, C₁-C₆cycloalkyl, C₂-C₅ heterocycloalkyl, C₆-C₁₀ aryl, and C₁-C₉ heteroaryl;
each R¹⁷ is independently halogen, hydroxyl, C₁-C₆ alkyl, C₃-C₆ cycloalkyl, C₁-C₆ alkoxy, C₁-C₆ haloalkyl, amino, cyano, C₁-C₆ heteroalkyl, C₁-C₆ hydroxyalkyl, —O—C₁-C₆haloalkyl, or —S—C₁-C₆haloalkyl;
R²⁸ and R²⁹ are each independently hydrogen, hydroxyl, C₁-C₆ alkyl, C₁-C₆, alkoxy, cyano, nitro, or C₃-C₆ cycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, cycloalkyl, heterocycloalkyl, aryl, heteroaryl, —C₁-C₆alkylene-cycloalkyl, —C₁-C₆alkylene-heterocycloalkyl, —C₁-C₆alkylene-aryl, —C₁-C₆alkylene-heteroaryl, —C₁-C₆heteroalkylene-cycloalkyl, —C₁-C₆heteroalkylene-heterocycloalkyl, —C₁-C₆heteroalkylene-aryl, —C₁-C₆alkylene-heteroaryl wherein each of the alkyl, alkoxy, heteroalkyl, cycloalkyl, heterocycloalkyl, aryl, and heteroaryl is optionally substituted with one, two, or three groups selected from halogen, —CN, hydroxy, C₁-C₆, alkyl, C₁-C₆ haloalkyl, C₁-C₆ alkoxy, C₃-C₆, cycloalkyl, C₂-C₅heterocycloalkyl, C₆-C₁₀ aryl, and C₁-C₉heteroaryl; and
R³³ is hydrogen, C₁-C₆ alkyl, C₁-C₆ heteroalkyl, or C₃-C₆ cycloalkyl, wherein each of the alkyl, heteroalkyl, and cycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉heteroaryl.

26. The radiopharmaceutical conjugate of claim 24 or claim 25, wherein the radionuclide is covalently bound to the structure of Formula (IV).

27. The radiopharmaceutical conjugate of any one of claims 24-26, wherein at least one of R²¹, R²², R²³, R²⁴, and R³⁰ comprises the radionuclide.

28. The radiopharmaceutical conjugate of any one of claims 24-27, wherein the radionuclide is fluorine-18 (¹⁸F), iodine-131 (¹³¹I), iodine-123 (¹²³I), iodine-124 (¹²⁴I), iodine-125 (¹²⁵I), or astatine-211 (²¹¹At).

29. The radiopharmaceutical conjugate of any one of claims 24-28, wherein the radioisotope is iodine-131 (¹³¹I).

30. The radiopharmaceutical conjugate of any one of claims 24-29, wherein
R²⁴ is

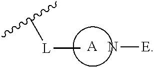

31. The radiopharmaceutical conjugate of any one of claims 24-30, wherein
E³ is C═O;
J is NR³⁰;
M is N, NR³³, or CR³³; and
R³³ is hydrogen or C₁-C₆ alkyl.

32. The radiopharmaceutical conjugate of any one of claims 24-31, wherein
the targeting ligand comprises a structure of Formula (IVa), or a salt or solvate thereof,

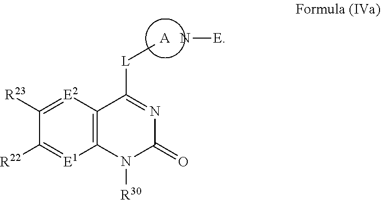

33. The radiopharmaceutical conjugate of any one of claims 24-32, wherein the targeting ligand comprises a structure of Formula (IVb) or Formula (IVc), or a salt or solvate thereof,

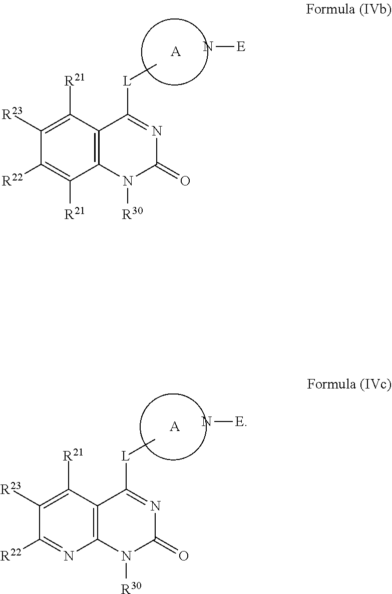

34. The radiopharmaceutical conjugate of any one of claims 24-33, wherein
each R² is independently hydrogen, hydroxyl, cyano, halogen, C₁-C₆alkyl, C₁-C₄haloalkyl, C₁-C₄alkoxyl, or C₁-C₄heteroalkyl;
R²² is halogen, C₁-C₆alkyl, C₂-C₃alkenyl, C₂-C₃alkynyl, OR²², N(R^{22′})₂, C₁-C₆cycloalkyl, C₂-C₅heterocycloalkyl, C₆-C₁₄alkyl, or C₂-C₁₄heteroaryl, each of the alkyl, alkenyl, alkynyl, cycloalkyl, heterocycloalkyl, aryl, and heteroaryl is optionally substituted, and each R is independently hydrogen, C₁-C₆alkyl, C₃-C₆cycloalkyl, C₂-C₅heterocycloalkyl, C₂-C₃alkenyl, C₂-C₃alkynyl, C₆-C₁₄aryl, C₂-C₁₄heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form an optionally substituted 3-7-membered ring; and
R²³ is hydrogen, halogen, C₁-C₆alkyl, C₁-C₃alkoxy, C₁-C₆cycloalkyl, C₂-C₉heterocycloalkyl, C₂-C₃alkenyl, C₂-C₃alkynyl, C₆—C, aryl, or C₂-C₁₄ heteroaryl, wherein each of the alkyl, alkoxy, cycloalkyl, heterocycloalkyl, alkenyl, alkynyl, aryl, and heteroaryl is optionally substituted; and
R³⁰ is halogen, cyan, C₁-C₆ alkyl, C₁-C₆ alkoxy, C₁-C₆ heteroalkyl, cycloalkyl, heterocycloalkyl, aryl, or heteroaryl, wherein each of the alkyl, heteroalkyl, cycloalkyl, heterocycloalkyl, aryl, and heteroaryl is optionally substituted.

35. The radiopharmaceutical conjugate of any one of claims 24-34, wherein
R²¹ is hydrogen;
R²² is C₁-C₃aryl or C₂-C₁₄ heteroaryl, each of which is optionally substituted; and
R²³ is halogen, C₁-C₃alkyl, C₁-C₃haloalkyl; and
R³⁰ is C₆-C₁₄aryl or C₂-C₁₄heteroaryl, each of which is optionally substituted with one or more substituents selected from halogen, C₁-C₃alkyl, C₁-C₃alkoxyl, or cyano.

36. The radiopharmaceutical conjugate of any one of claims 24-35 wherein
R²² is phenyl, optionally substituted with one or more substituents selected from C₁-C₃alkyl, halogen, and hydroxyl; or
R²² is bicyclic heteroaryl, optionally substituted with one or more substituents selected from C₁-C₃alkyl, halogen, and hydroxyl.

37. The radiopharmaceutical conjugate of any one of claims 24-36, wherein
ring A is piperazinyl substituted with one to three substituents selected from halogen and C₁-C₃alkyl;
L is a bond, C₁-C₃alkylene, S, O, or NH; and
X is C(═O).

38. The radiopharmaceutical conjugate of any one of claims 24-37, wherein L is a bond.

39. The radiopharmaceutical conjugate of any one of claims 24-38, wherein
R⁵ is hydrogen, halogen, or a C₁-C₃alkyl optionally substituted by one or more hydroxyl and/or halogen,
R⁶ is hydrogen, cyano, halogen, optionally substituted C₁-C₆ alkyl, optionally substituted C₁-C₆ alkoxyl, optionally substituted C₁-C₆ heteroalkyl, optionally substituted C₃-C₆ Cycloalkyl, or optionally substituted C₂-C₆ heterocycloalkyl; and
R⁷ is hydrogen, cyan, halogen, optionally substituted C₁-C₆ alkyl, optionally substituted C₁-C₆ alkoxyl, or optionally substituted C₁-C₆ heteroalkyl.

40. The radiopharmaceutical conjugate of any one of claims 24-39, wherein E is

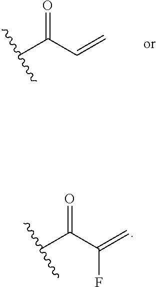

41. The radiopharmaceutical conjugate of any one of claims 24-40, wherein
R³⁰ is phenyl or 6-membered heteroaryl, optionally substituted with one or more of C₁-C₃alkyl.

42. The radiopharmaceutical conjugate any one of claims 24-41, wherein R²² comprises the radionuclide.

43. The radiopharmaceutical conjugate of claim 42, wherein
R²² is 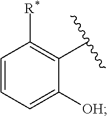
and
R* is fluorine-18(¹⁸F), iodine-131 (¹³¹I), iodine-123 (¹²³I), iodine-124 (¹²⁴I), iodine-125 (¹²⁵I), or astatine-211 (²¹¹At).

44. The radiopharmaceutical conjugate of any one of claims 24-43, wherein
the radiopharmaceutical conjugate has a structure of

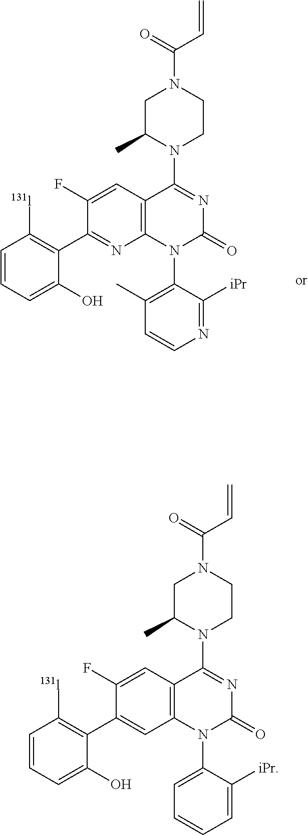

45. The radiopharmaceutical conjugate of any one of claims 24-41, wherein
R²³ comprises the radionuclide; and
R²³ is halogen and the halogen is the radionuclide selected from fluorine-18 (¹⁸F), iodine-131 (¹³¹I), iodine-123 (¹²³I), iodine-124 (¹²⁴I), iodine-125 (¹²⁵I), and astatine-211 (²¹¹At).

46. The radiopharmaceutical conjugate of claim 45, wherein R²³ is ¹³¹I.

47. The radiopharmaceutical conjugate of any one of claims 24-41, 45, or 46, wherein
the radiopharmaceutical conjugate has a structure of

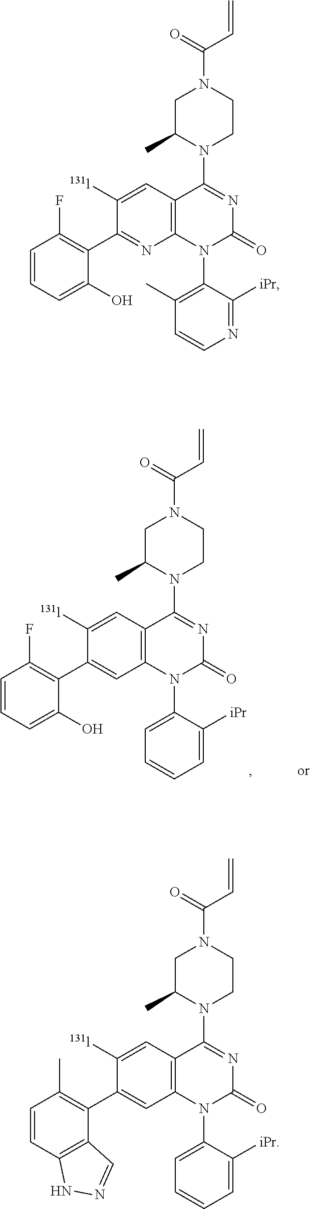

48. The radiopharmaceutical conjugate of any one of claims 1-6, 9-15, 19-21, or 24-42, wherein
the radiopharmaceutical conjugate further comprises;
a linker covalently bonded to the radionuclide and to the radiolabeled compound, or a salt or solvate thereof.

49. The radiopharmaceutical conjugate of claim 48, wherein the linker is a brush border enzyme-cleavable linker, a hepatocyte-cleavable linker, a cytochrome P450-substrate, an esterase-cleavable linker, or a peptidase-cleavable linker.

50. A radiolabeled compound comprising a structure of Formula (IIIb), or a salt or solvate thereof, 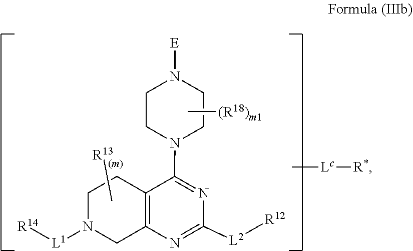
wherein
L^{C} is a linker comprising 1 to 20 groups independently selected from —CR^{b}R^{b}—, —C(═O)—, —S(═O)—, —S(═O)₂—, —NR^{a}—, 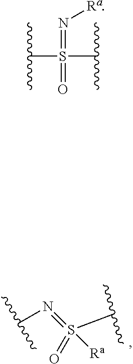
—CR^{b}═CR^{b}—, —C═C—, —O—, —S—, —C(═O)O—, —OC(═O)—, —C(═O)NR^{a}—, —NR—C(═O), —S(O)₂NR^{a}—, —NR^{a}S(═O)₂—, —NR^{a}C(═O)NR^{a}—, —NR^{a}C(═O)O—, —OC(═O)NR^{a}—, arylene, and heteroarylene;
each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
each R^{b} is independently hydrogen, halogen, —CN, —NO₂, OR^{a}, —SR^{a}, C₁-C₆alkyl, C₁-C₄haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
L¹ is a bond, —C(═O)—, or optionally substituted C₁-C₃ alkylene, wherein the alkylene is optionally substituted with one, two, or three groups selected from halogen, —CN, hydroxy, C₁-C₆ alkyl, C₁-C₆ haloalkyl, C₁-C₆ alkoxy, C₃-C₈cycloalkyl, and C₂-C₅heterocycloalkyl;
L² is a bond, —C(═O)—, O, S or NR¹⁵;
E is 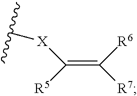
X is C(═O), P(═O)OR², C(═S), or S(═O)ₙ, where n is 1 or 2;
R² is hydrogen or substituted or unsubstituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₃ alkoxy, C₃-C₆ cycloalkyl, and C₂-C₅ heterocycloalkyl;
R⁵ is hydrogen, cyano, halogen, C₁-C₆ alkyl, C₁-C₆ alkoxyl, C₁-C₆ heteroalkyl, C₃-C₆ cycloalkyl, or C₁-C₅ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₁-C₆cycloalkyl, and C₁-C₅ heterocycloalkyl;
R⁷ is hydrogen, cyano, halogen, C₁-C₆ alkyl, C₁-C₆ alkoxyl, C₁-C₆ heteroalkyl, C₃-C₆ cycloalkyl, C₂-C₉ heterocycloalkyl, or C₁-C₉heteroaryl, wherein each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₆ heterocycloalkyl; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyano, halogen, C₁-C₆ alkyl, C₁-C₆ alkoxyl, C₁-C₆ heteroalkyl, C₃-C₆ cycloalkyl, C₂-C₆ heterocycloalkyl, or C₁-C₆ heteroaryl, wherein, each of the alkyl, alkoxyl, heteroalkyl, cycloalkyl, heterocycloalkyl, or heteroaryl is optionally substituted with one, two, or three groups selected from halogen, —CN, hydroxy, C₁-C₆ alkyl, C₁-C₆ haloalkyl, C₁-C₆ alkoxy, C₃-C₆ cycloalkyl, and C₂-C₅ heterocycloalkyl;
R¹² is C₁-C₆ alkyl, C₁-C₆ heteroalkyl, -L³—NR¹⁵SR^{15′}, C₂-C₆heterocycloalkyl, -L³-C₂-C₁₂heterocycloalkyl, C₃-C₁₅cycloalkyl, -L³-C₃-C₁₅cycloalkyl, aryl, C₁-C₉heteroaryl, -L³-aryl, or -L³-C₁-C₉ heteroaryl, wherein each of the heterocycloalkyl, cycloalkyl, aryl, heteroaryl, alkyl or heteroalkyl is optionally substituted with one, two, three or four groups selected from R¹⁹;
L³ is C₁-C₄ alkylene or C₁-C₄ heteroalkylene, each of which is optionally substituted with one, two, or three groups selected from R¹⁹;
each R¹³ is independently OH, halogen, oxo, substituted or unsubstituted C₁-C₆ alkyl, or substituted or unsubstituted C₁-C₆ heteroalkyl, wherein each of the alkyl and heteroalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, and C₂-C₅heterocycloalkyl;
R¹⁴ is hydrogen, C₁-C₃ cycloalkyl, C₂-C₁₂heterocycloalkyl, aryl, or C₁-C₉heteroaryl, wherein each of the cycloalkyl, heterocycloalkyl, aryl, and heteroaryl is optionally substituted with one, two, three or four groups selected from R¹⁶;
each R¹⁵ is independently hydrogen or C₁-C₃ alkyl;
each R^{15′} is independently hydrogen, acyl, C₁-C₃ alkyl, C₁-C₃ heteroalkyl or C₁-C₃ hydroxyalkyl;
m is 0, 1, or 2;
each R¹⁷ is independently halogen, hydroxyl, C₁-C₆ alkyl, C₃-C₆ cycloalkyl, C₁-C₆ alkoxy, C₁-C₆ haloalkyl, amino, cyano, C₁-C₆ heteroalkyl, C₁-C₆ hydroxyalkyl, —O—C₁-C₆haloalkyl, or —S—C₁-C₆haloalkyl;
each R^{1R} is independently halogen, oxo, C₁-C₆ alkyl, C₁-C₆ aminoalkyl, C₁-C₆ haloalkyl, C₂-C₆ alkenyl, C₂-C₆ alkynyl, C₁-C₆ heteroalkyl, —CN, —C(O)OR′, —C(O)N(R¹⁵)(R^{15′}), —N(R¹⁰)(R^{10′}), or OR^{a}, and wherein each of the alkyl, aminoalkyl, haloalkyl, alkenyl, alkynyl, or heteroalkyl is optionally substituted with one, two, or three groups selected from halogen, —CN, —NO₂, amino, hydroxy, C₁-C₆ alkyl, C₁-C₆ haloalkyl, C₁-C₆ alkoxy, C₁-C₆cycloalkyl, and C₂-C₃ heterocycloalkyl;
m1 is 0, 1, 2, or 3;
R¹⁶ and R¹⁹ are each independently selected from halogen, oxo, —CN, C₁-C₆ alkyl, C₂-C₆ alkenyl, C₂-C₆ alkynyl, C₁-C₆ heteroalkyl, C₃-C₁₀cycloalkyl, —C₁₋₃alkylene-C₃-C₁₀cycloalkyl, C₂-C₉ heterocycloalkyl, —C₁₋₃alkylene-C₁₋₉heterocycloalkyl, C₆-C₁₀ aryl, C₁₋₃alkylene-C₆₋₁₀aryl, C₁-C₉heteroaryl, —C₁₋₃alkylene-C₂₋₉heteroaryl, —OR¹⁰, —SR¹⁰, —N(R¹⁰)(R^{10′}), —C(═O)OR¹⁰, —OC(═)N(R¹⁹)(R¹⁰), —N(R^{10′})C(═O)N(R¹⁰)(R^{10′}), —N(R^{10′})C(═O)OR^{a}, —N(R¹⁰)S(═O)₂R¹¹, —C(═O)R¹¹, —S(═O)R¹¹, —OC(═O)R¹¹, —C(═O)N(R¹⁰)(R^{10′}), —C(═O)C(═O)N(R¹⁰)(R^{10′}), —N(R^{10′})C(O)R¹¹, —S(O)₂R¹¹, —S(O)₂N(R¹⁰)(R^{10′}), —S(═O)(═NH)N(R¹⁰)(R^{10′}), —CH₂C(O)N(R¹⁰)(R^{10′}), —CH₂N(R^{10′})C(═O)R¹¹, —CH—S(═O)₂R¹¹, and —CH—S(═O), N(R¹⁰)(R^{10′}), wherein the alkyl, alkenyl, alkynyl, cycloalkyl, —C₁₋₃alkylene-C₆₋₁₀cycloalkyl, heterocycloalkyl, —C₁₋₃alkylene-C₂-C₉heterocycloalkyl, aryl, —C₁₋₃alkylene-C₆₋₁₀aryl, heteroaryl and —C₁-C₃alkylene-C₂₋₉ heteroaryl are optionally substituted with one, two, three, or four groups independently selected from halogen, oxo, —CN, C₁-C₆ alkyl, C₁-C₆ haloalkyl, C₁-C₆ alkoxy, C₁-C₆, alkyloxy, C₁-C₆ haloalkoxy, C₃-C₁₀ cycloalkyl, C₂-C₉heterocycloalkyl, C₆-C₁₀ aryl, C₁-C₉heteroaryl, —OR¹⁰, —SR¹⁰, —N(R¹⁰)(R^{10′}), —C(═O)OR¹⁰, —OC(═O)N(R¹⁰)(R^{10′}), —N(R)C(═O)N(R¹⁰)(R^{10′}), —N(R^{10′})C(═O)OR^{a}, —N(R^{10′})S(═O)₂R¹¹, —C(═O)R¹¹, —S(O)R¹¹, —OC(═O)R¹¹—C(═O)N(R¹⁰)(R^{10′}), —C(═O)C(═O)N(R¹⁰)(R^{10′}), —N(R^{10′})C(═O)R¹¹, —S(═O)₂R¹¹, —S(O)₂N(R¹⁰)(R^{10′})—, S(═O)(═NH)N(R¹⁰)(R^{10′}), —CH—C(═O)N(R¹⁰)(R^{10′}), —CH₂N(R¹⁰)C(═O)R¹¹, —CH₂S(═O)₂R¹¹, and —CH₂S(═O)₂N(R¹⁰)(R^{10′});
each R¹⁰ is independently selected from hydrogen, C₁-C₆ alkyl, C₁-C₆ haloalkyl, C₂-C₆ alkenyl, C₁-C₆, alkynyl, C₃-C₆ cycloalkyl, C₂ heterocycloalkyl, C₆-C₁₀ aryl, and C₁-C₉ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆ haloalkyl, C₁-C₆, alkoxy, C₃-C₆ cycloalkyl, C₁-C₉ heterocycloalkyl, C₆-C₁₀ aryl, and C₁-C₉heteroaryl;
each R^{10′} is independently selected from hydrogen, C₁-C₆ alkyl, and C₁-C₆, haloalkyl, and
each R¹¹ is independently selected C₁-C₆ alkyl, C₂-C₆ alkenyl, C₂-C₆ alkynyl, C₃-C₅cycloalkyl, C₂-C₉ heterocycloalkyl, C₆-C₁₀ aryl, and C₁-C₉ heteroaryl, wherein the alkyl, alkenyl, alkynyl, cycloalkyl, heterocycloalkyl, aryl, and heteroaryl are optionally substituted with one, two, or three groups selected from halogen, —CN, hydroxy, C₁-C₆ alkyl, C₁-C₆haloalkyl, C₁-C₆ alkoxy, C₃-C₆ cycloalkyl, C₁-C₄ heterocycloalkyl, C₆-C₁₀ aryl, and C₁-C₉heteroaryl; and
R* is fluorine-18 (¹⁸F) iodine-131 (¹³¹I), iodine-123 (¹²³I), iodine-124 (¹²⁴I), iodine-125 (¹²⁵I), or astatine-211 (²¹¹At).

51. The radiolabeled compound of claim 50, or a salt or solvate thereof, wherein the radiolabeled compound comprises a structure of Formula (IIIc);

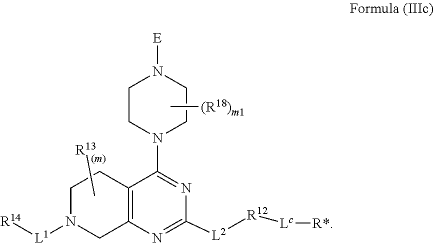

52. A radiolabeled compound comprising a structure of Formula (IVd), or a salt or solvate thereof, 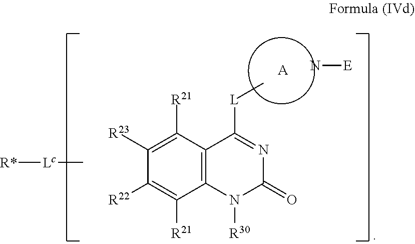
wherein
L^{C} is a linker comprising 1 to 20 groups independently selected from —CR^{b}R^{b}—, —C(═O)—, —S(═O)—, —S(═O)—, —NR^{a}— 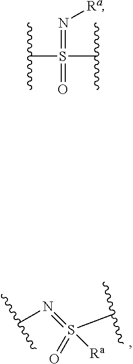
—CR^{b}═CR^{b}—, —C≡C—, —O—, —S—, —C(═O)O—, —OC(═O)—, —C(═O)NR^{a}—, —NR^{a}C(═O)—, —S(O)₂NR^{a}—, —NR^{a}S(═O)₂—, —NR^{a}C(═O)NR^{a}—, —NR^{a}C(═O)O—, —OC(═O)NR^{a}—, arylene, and heteroarylene;
each R^{a} is independently hydrogen, halogen, C₁-C₄alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₉heterocycloalkyl, aryl, or heteroaryl;
each R^{b} is independently hydrogen, halogen, —CN, —NO₂, —OR^{a}, —SR^{a}, C₁-C₆alkyl, C₁-C₆-haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl;
R²¹ is independently hydrogen, hydroxyl, cyano, halogen, C₁-C₆ alkyl, C₁-C₆ alkoxyl, or C₁-C₆ heteroalkyl, wherein each of the alkyl, alkoxy, and heteroalkyl is optionally substituted with one, two, or three groups selected from halogen, —CN, —NO₂, amino, hydroxy, C₁-C₆ alkyl, C₁-C₆ haloalkyl, C₁-C₆, alkoxy, C₃-C₆cycloalkyl, C₂-C₅heterocycloalkyl, C₆-C₁₀ aryl, and C₁-C₉ heteroaryl;
R²² is C₁-C₆ alkyl, C₂-C₆ alkenyl, C₂-C₆ alkynyl, OR^{22′}, N(R^{22′})₂, cycloalkyl, heterocycloalkyl, aryl, or heteroaryl, wherein each of the alkyl, alkylene, alkenyl, alkyl, cycloalkyl, heterocycloalkyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆, alkoxy, C₃-C₆ cycloalkyl, and C₂-C₅ heterocycloalkyl, and each R²² is independently hydrogen, C₁-C₆ alkyl, cycloalkyl, heterocycloalkyl, C₂-C₆ alkenyl, C₁-C₆ alkynyl, aryl, or heteroaryl, wherein each of the alkyl, cycloalkyl, heterocycloalkyl, alkenyl, alkynyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆, alkoxy, C₃-C₆cycloalkyl, C₂-C₅ heterocycloalkyl, C₆-C₁₀ aryl, and C₁-C₉heteroaryl; or two R^{22′} together with the nitrogen atom to which they are attached, form an optionally substituted 3-7-membered ring with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉ heteroaryl;
R²³ is hydrogen, halogen, C₁-C₆ alkyl, C₁-C₆ alkoxy, C₁-C₆, alkenyl, C₂-C₆ alkynyl, cycloalkyl, heterocycloalkyl, aryl, heteroaryl, wherein each of the alkyl, alkoxy, cycloalkyl, heterocycloalkyl, alkenyl, alkynyl, aryl, and heteroaryl is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₁-C₅heterocycloalkyl, C₆-C₁₀ aryl, and C₁-C₉ heteroaryl;
ring A is an optionally substituted 4-7 membered monocyclic ring or optionally substituted 6-11 membered bicyclic, bridged, fused, or spiro ring each of which is optionally substituted with one, two, or three groups selected from halogen, —CN, —NO₂, amino, hydroxy, C₁-C₆ alkyl, C₁-C₆ haloalkyl, C₁-C₆ alkoxy, C₃-C₆cycloalkyl, C₂-C₆ heterocycloalkyl, C₆-C₁₀ an 1, and C₁-C₉ heteroaryl;
L is a bond, C₁-C₆ alkylene, C₁-C₆ heteroalkylene, S, O, or NH;
E represents a structure of Formula (Ic), 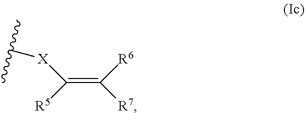
wherein,
X is C(═O), P(═O)OR′, C(═S), or S(═O)ₙ, where n is 1 or 2;
R² is hydrogen or substituted or unsubstituted C₁-C₃ alkyl, wherein the alkyl is optionally substituted with one, two, or three groups selected from halogen, —CN, hydroxy, C₁-C₃ alkyl, C₁-C₃ haloalkyl, C₁-C₆ alkoxy, C₁-C₆ cycloalkyl, and C₂-C₅ heterocycloalkyl, C₆-C₁₀ aryl, and C₁-C₉ heteroaryl;
R⁵ is hydrogen, cyano, halogen, C₁-C₆ alkyl, C₁-C₆ alkoxyl, C₁-C₆ heteroalkyl, C₃-C₆ cycloalkyl, or C₂-C₆ heterocycloalkyl, each of the alkyl, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅ heterocycloalkyl, C₆-C₁₀ aryl, and C₁-C₉heteroaryl;
R⁷ is hydrogen, cyano, halogen, C₁-C₆ alkyl, C₁-C₆ alkoxyl, C₁-C₆ heteroalkyl, C₃-C₇ cycloalkyl, C₂-C₇ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅heterocycloalkyl, C₆-C₁₀ aryl, and C₁-C₉heteroaryl; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted with one, two or three groups selected from R¹⁷;
R⁶ is hydrogen, cyano, halogen, C₁-C₆ alkyl, C₁-C₆ alkoxyl, C₁-C₆ heteroalkyl, C₃-C₆ cycloalkyl, C₁-C₃ heterocycloalkyl, heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted with one, two, or three groups selected from halogen, —CN, hydroxy, C₁-C₆ alkyl, C₁-C₆ haloalkyl, C₁-C₆ alkoxy, C₃-C₆cycloalkyl, C₂-C₅heterocycloalkyl, C₂-C₁₀ aryl, and C₁-C₉heteroaryl;
each R¹⁷ is independently halogen, hydroxyl, C₁-C₆ alkyl, C₁-C₆ cycloalkyl, C₁-C₆ alkoxy, C₁-C₆ haloalkyl, amino, cyano, C₁-C₆ heteroalkyl, C₁-C₆ hydroxyalkyl, —O—C₁-C₆haloalkyl, or —S—C₁-C₆haloalkyl; and
R* is fluorine-18 (¹⁸F), iodine-131 (¹³¹I), iodine-123 (¹²³I), iodine-124 (¹²⁴I), iodine-125 (¹²⁵I), or astatine-211 (²¹¹At).

53. The radiolabeled compound of claim 52, wherein the radiolabeled compound comprises a structure of Formula (IVe),

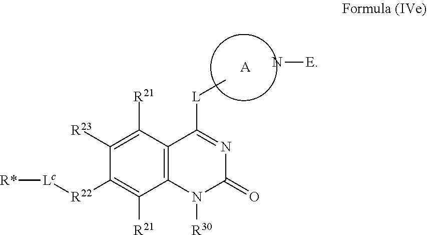

54. The radiolabeled compound of any one of claims 50-53, wherein 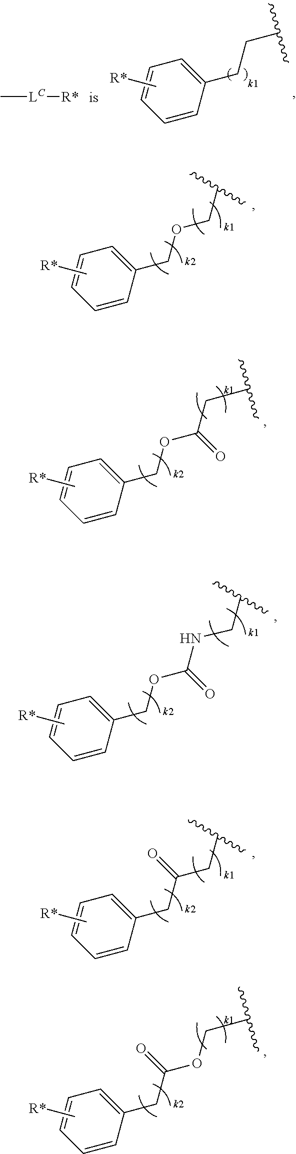 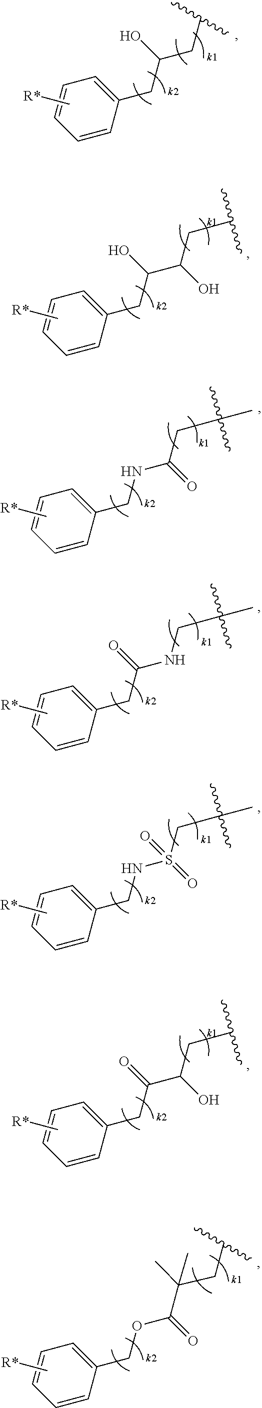 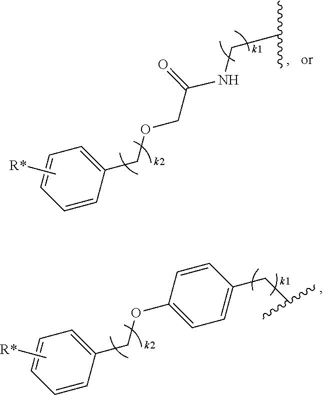
wherein each k1 and k2 is independently 0 or an integer selected from 1 to 10.

55. The radiolabeled compound of claim 54, wherein each k1 and k2 is independently 0 or an integer selected from 1 to 5.

56. The radiolabeled compound of any one of claims 50-55, wherein

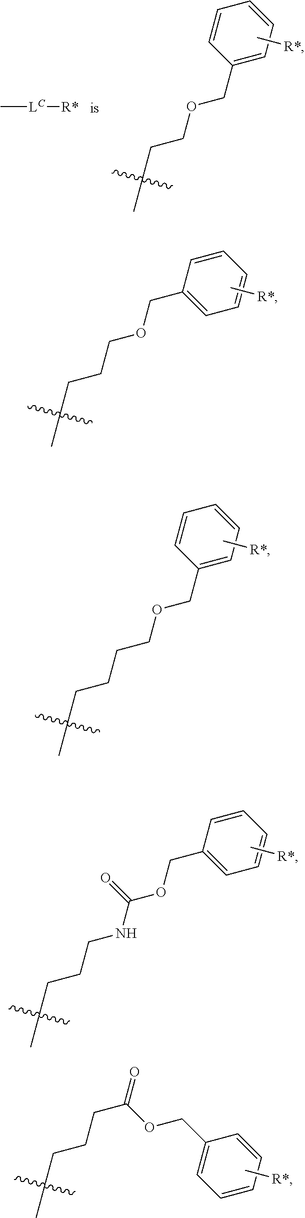

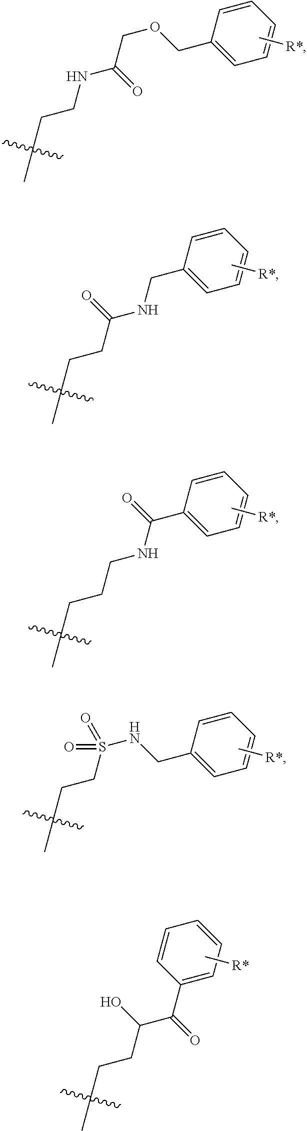

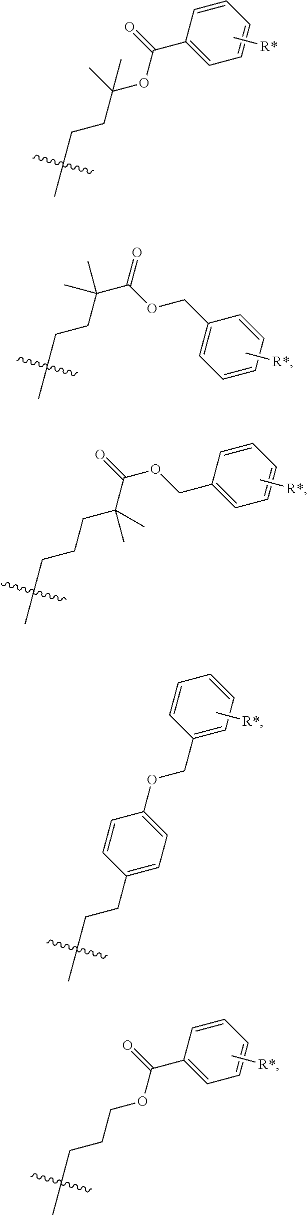

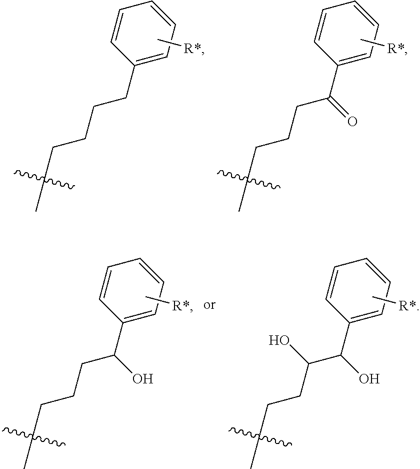

57. The radiolabeled compound of any one of claims 50, 51, or 54-56, wherein the radiolabeled compound is:

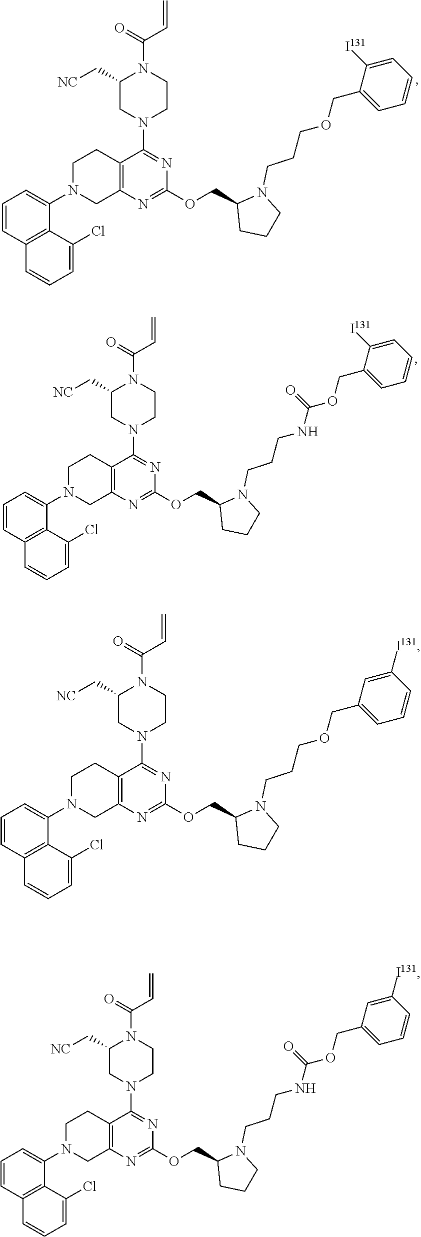

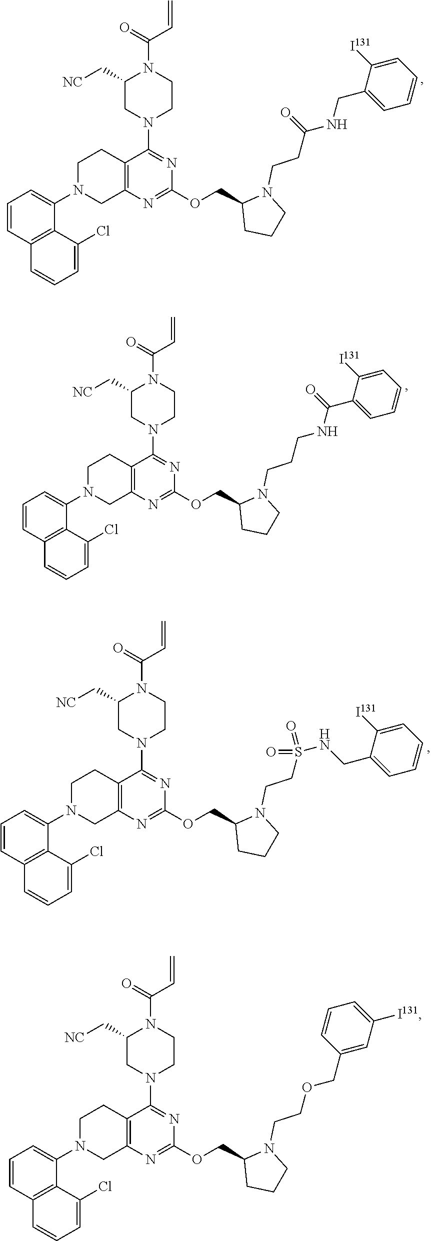

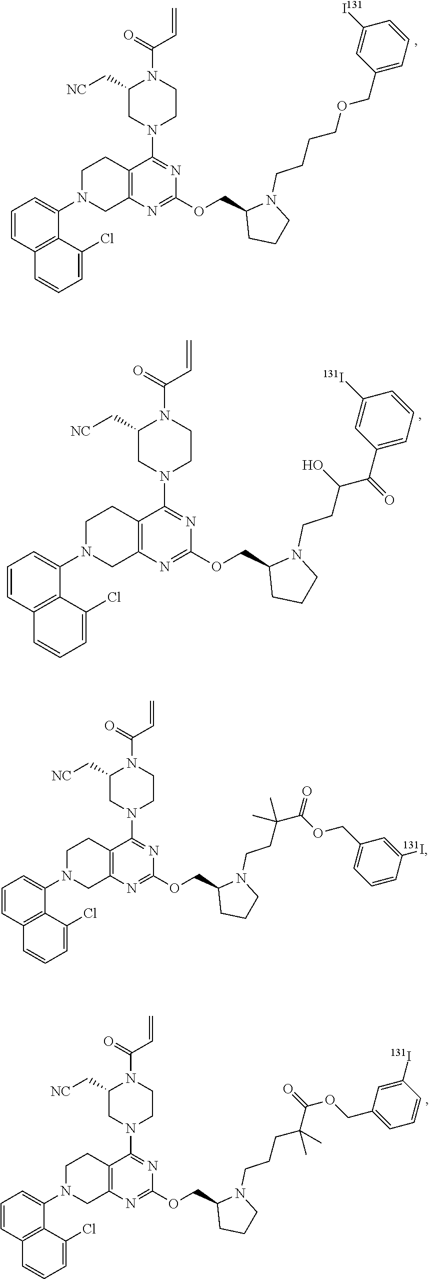

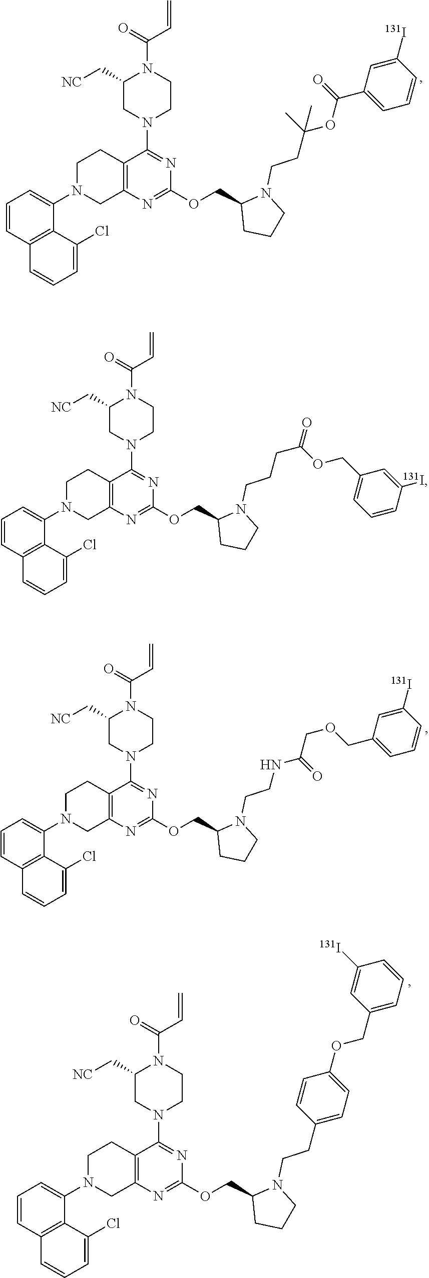

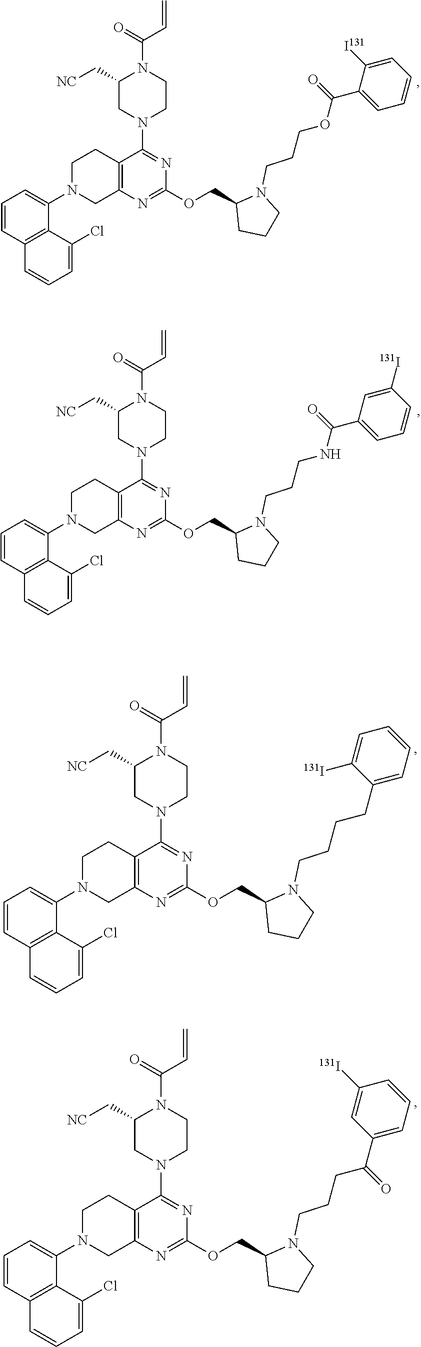

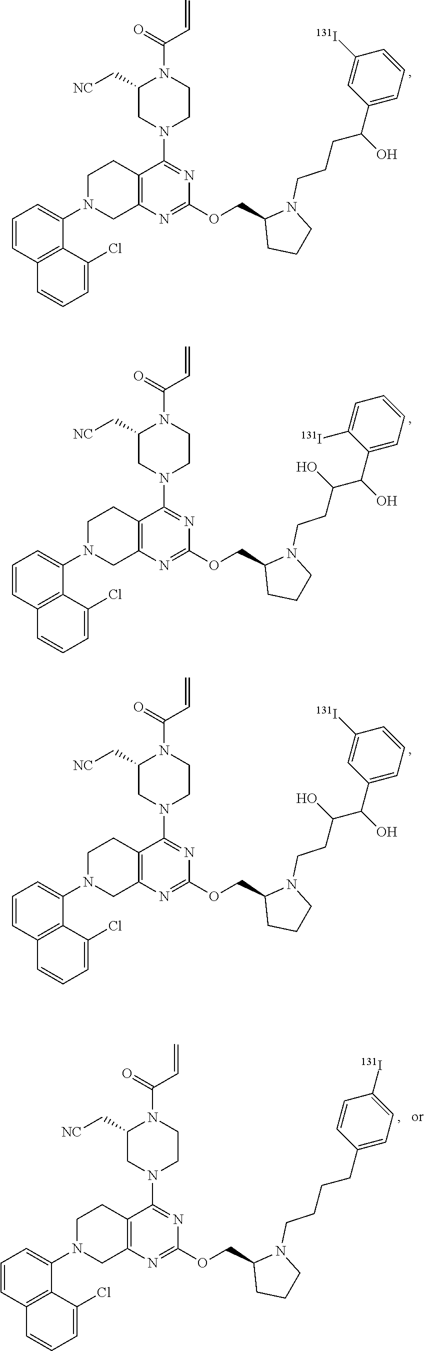 [SMILES: C=CC(=O)N1CCN(c2nc(OC[C@@H]3CCCN3CCCC(O)c3cccc([131I])c3)nc3c2CCN(c2cccc4cccc(Cl)c24)C3)C[C@@H]1CC#N ; C=CC(=O)N1CCN(c2nc(OC[C@@H]3CCCN3CCC(O)C(O)c3ccccc3[131I])nc3c2CCN(c2cccc4cccc(Cl)c24)C3)C[C@@H]1CC#N ; C=CC(=O)N1CCN(c2nc(OC[C@@H]3CCCN3CCC(O)C(O)c3cccc([131I])c3)nc3c2CCN(c2cccc4cccc(Cl)c24)C3)C[C@@H]1CC#N ; C=CC(=O)N1CCN(c2nc(OC[C@@H]3CCCN3CCCCc3ccc([131I])cc3)nc3c2CCN(c2cccc4cccc(Cl)c24)C3)C[C@@H]1CC#N]

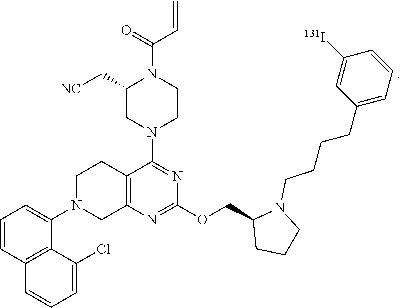 [SMILES: C=CC(=O)N1CCN(c2nc(OC[C@@H]3CCCN3CCCCc3cccc([131I])c3)nc3c2CCN(c2cccc4cccc(Cl)c24)C3)C[C@@H]1CC#N]

58. The radiolabeled compound of any one of claims 52, 53, or 54-56, wherein the radiolabeled compound is;

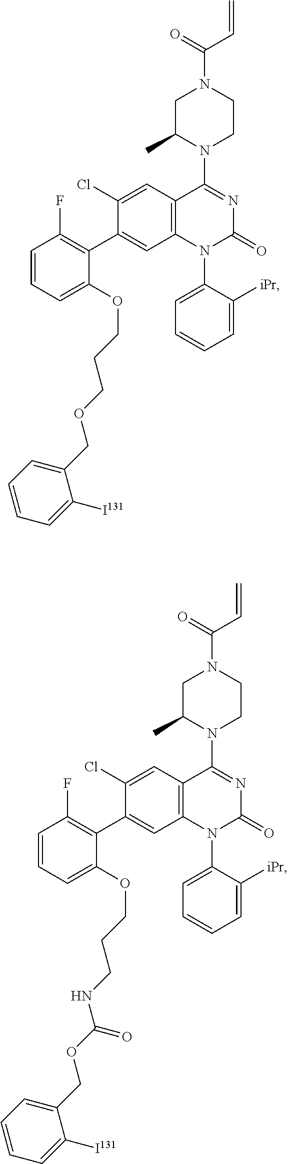

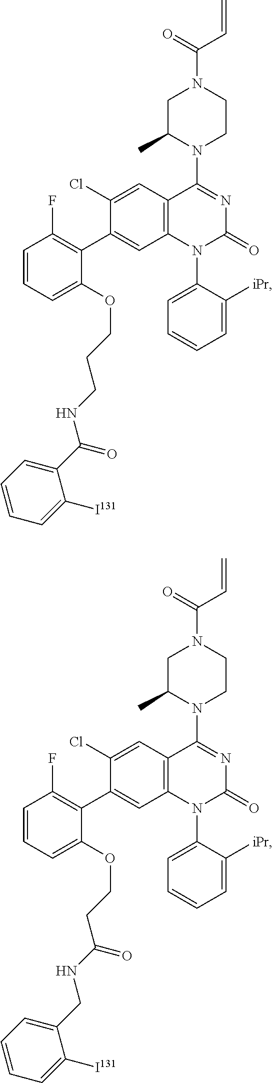

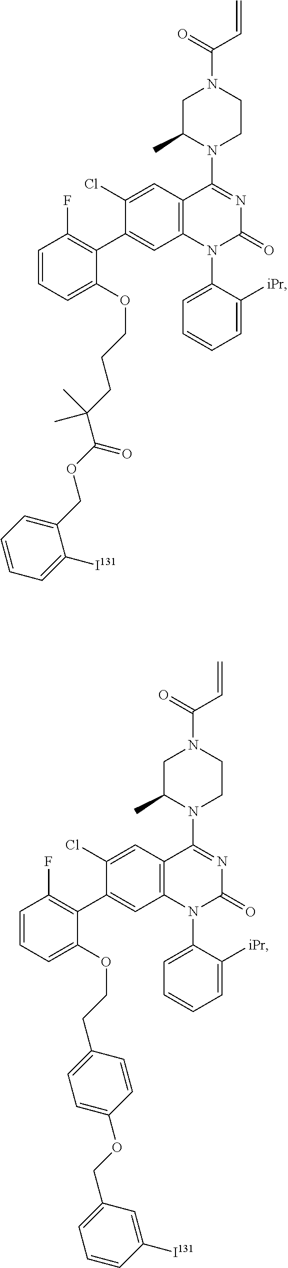

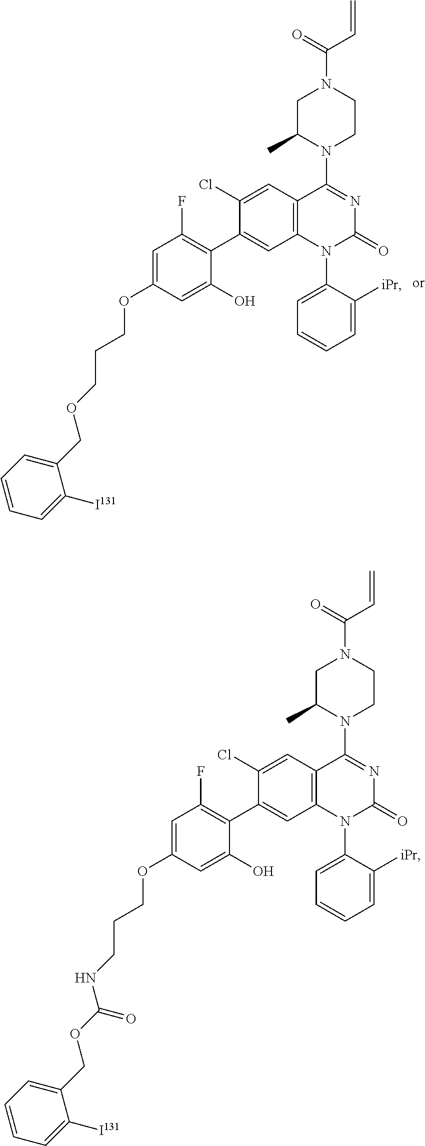

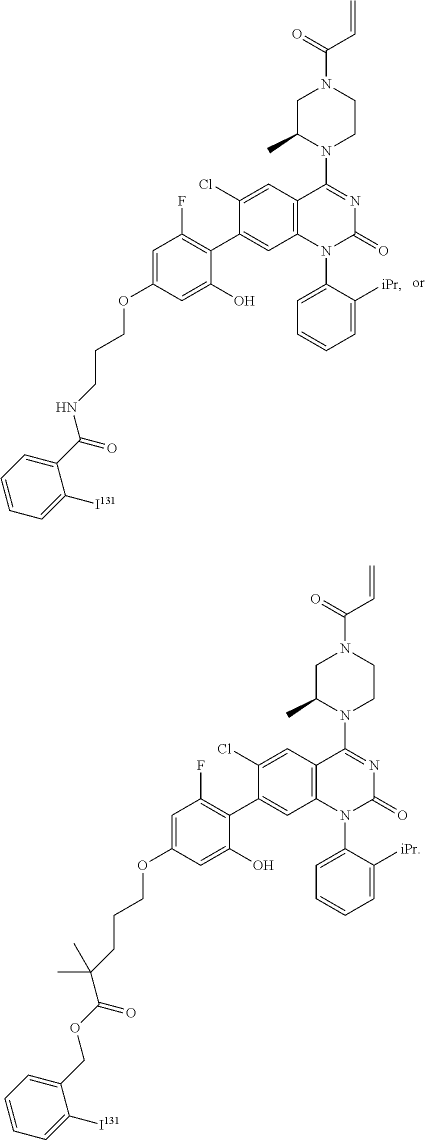

59. The radiolabeled compound of claim 1 or 24, further comprising:
(a) a linker; and
(b) a metal chelator.

60. A conjugate having a structure of Formula (X): 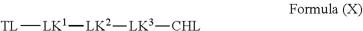
wherein,
TL represents a targeting ligand;
CHL represents a metal chelator, optionally bound to a radionuclide; and
each of LK¹, LK², and LK³independently selected from substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₁-C₁₂ alkynylene, substituted or unsubstituted cycloalkylene, substituted or unsubstituted heterocycloalkylene, —(CH₂CH—O)_{q}—, —(OCH₂CH₂)_{q}—, —O—, —S—, —S(═O)—, —S(O)₂, —S(O)(═NR¹⁵)—, —C(═O)—, —C(═N—OR)—, —C(═O)O—, —OC(═O)—, —C(═O)C(═O)—, —C(═O)NR^{a}—, —NR^{LK}C(═O)—, —OC(═O)NR—, —NR^{LK}C (═O)O—, —NR^{LK}C(═O)NR^{LK}, —C(═O)NR^{LK}C(O)—, —S(═O)₂NR^{LK}, —NR^{LK}S(O)₂, —NR^{LK}—, —N(OR^{LK})—, and a bond;
each R^{LK} is independently hydrogen or substituted or unsubstituted C₁-C₆alkyl; and
q is an integer selected from 1 to 10.

61. The conjugate of claim 60, wherein
LK¹ is substituted or unsubstituted C₁-C₁₂ alkylene or substituted or unsubstituted C₁-C₁₂ heteroalkylene.

62. The conjugate of claim 60 or 61, wherein
each of LK² and LK³ is independently a bond, C₁-C₆ alkylene, C₁-C₆ heteroalkylene, —(CH₂CH₂O)₁₋₃—, —(OCH₂CH₂)₁₋₃—, —O—, or —S—.

63. The conjugate of any one of claims 60-62, wherein
the conjugate of Formula (X) has the structure of Formula (X-III) 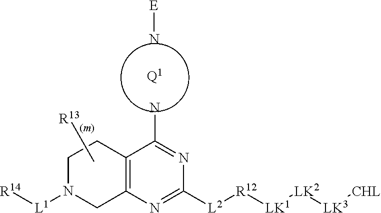
wherein
ring Q¹ is a 4-12 membered saturated or partially saturated monocyclic or bicyclic ring, wherein the monocyclic or bicyclic ring is optionally substituted;
L¹ is a bond, —C(═O)—, or optionally substituted C₁-C₃ alkylene;
L² is a bond, —C(═O)—, O, S or NR¹⁵;
E is 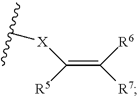
X is C(═O), P(O)OR⁼, C(═S), or S(═O)ₙ, where n is 1 or 2;
R² is hydrogen or optionally substituted C₁-C₆ alkyl;
R⁵ is hydrogen, cyano, halogen, C₁-C₆ alkyl, C₁-C₆ alkoxyl, C₁-C₆heteroalkyl, C₃-C₆ cycloalkyl, or C₂-C₄ heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃—C; cycloalkyl, C₂-C₇ heterocycloalkyl, C₁-C₉ heteroaryl, —C₁-C₆alkylene-heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆heteroalkyl, C₃-C₆ cycloalkyl, C₂-C₆ heterocycloalkyl, C₁-C₉heteroaryl, —C₁-C₆alkylene-C₁-C₉ heteroaryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R¹² is hydrogen, C₁-C₆ alkyl, C₁-C₆ heteroalkyl, -L³—NR¹⁵R^{15′}, C₂-C₁₂ heterocycloalkyl, -L³-C₂-C₁₂ heterocycloalkyl, C₃-C₁₀cycloalkyl, -L³-C₃-C₁₀cycloalkyl, aryl, C₁-C₉heteroaryl, -L³-aryl, or -L³-C₁-C₉heteroaryl, wherein each of the heterocycloalkyl, cycloalkyl, aryl, heteroaryl, alkyl or heteroalkyl is optionally substituted;
L³ is C₁-C₄ alkylene or C₁-C₄ heteroalkylene, each of which is optionally substituted each R¹³ is independently OH, halogen, oxo, substituted or unsubstituted C₁-C₆ alkyl, or substituted or unsubstituted C₁-C₆ heteroalkyl;
R¹⁴ is hydrogen, C₃-C₁₀cycloalkyl, C₂-C₁₂ heterocycloalkyl, aryl, or C₁-C₉heteroaryl, wherein each of the cycloalkyl, heterocycloalkyl, aryl, and heteroaryl is optionally substituted;
each R¹⁵ is independently hydrogen or C₁-C₃ alkyl;
each R^{a} is independently hydrogen, acyl, C₁-C₃ alkyl, C₁-C₃ heteroalkyl or C₁-C₃ hydroxyalkyl; and
m is 0, 1, or 2.

64. The conjugate of any one of claims 60-62, wherein the conjugate of Formula (X) has the structure of Formula (X-IV) 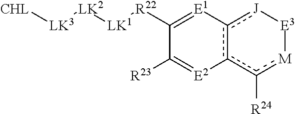
E¹ and E² are each independently N or CR²¹;
E³ is CR¹⁵R^{15′}, C═CR²⁸R²⁹, C═O, C═S, or C═NR²⁸;
J is N, NR³⁰, or CR³³;
M is N, NR³³, or CR³³; [symbol]
is a single or double bond as necessary to give every atom its normal valence,
R²¹ is independently hydrogen, hydroxyl, cyan, halogen, C₁-C₆ alkyl, C₁-C₆ alkoxyl, or C₁—C heteroalkyl, wherein each of the alkyl, alkoxy, and heteroalkyl are optionally substituted;
R²² is hydrogen, halogen, C₁-C₆ alkyl, C₂-C₆ alkenyl, C₂-C₆ alkynyl, OR²², N(R^{22′})₂, C₃-C₁₀cycloalkyl, C₂-C₁₂ heterocycloalkyl, aryl, or C₁-C₉ heteroaryl, wherein each of the alkyl, alkylene, alkenyl, alkynyl, cycloalkyl, heterocycloalkyl, aryl, and heteroaryl is optionally substituted, and each R^{22′} is independently hydrogen, C₁-C₆alkyl, C₃-C₁₀cycloalkyl, C₂-C₁₂heterocycloalkyl, C₂-C₆ alkenyl, C₂-C₆ alkynyl, aryl, or C₁-C₉ heteroaryl, wherein each of the alkyl, cycloalkyl, heterocycloalkyl, alkenyl, alkynyl, aryl, and heteroaryl is optionally substituted, or two R^{22′} together with the nitrogen atom to which they are attached, form a 3-7-membered ring;
R²³ is hydrogen, halogen, C₁-C₆ alkyl, C₁-C₃ alkoxy, C₂-C₆ alkenyl, C₂-C₆ alkynyl, C₃-C₁₀cycloalkyl, C₂-C₇ heterocycloalkyl, aryl, C₁-C₉ heteroaryl, wherein each of the alkyl, alkoxy, cycloalkyl, heterocycloalkyl, alkenyl, alkynyl, aryl, and heteroaryl is optionally substituted;
R²⁴ is 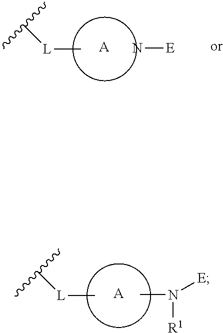
ring A is an optionally substituted 4-7 membered monocyclic ring or optionally substituted 6-11 membered bicyclic, bridged, fused, or spiro ring;
R¹ is hydrogen, optionally substituted C₁-C₆ alkyl, optionally substituted C₁-C₆ heteroalkyl, optionally substituted C₃-C₆ cycloalkyl, or optionally substituted C₁-C₅ heterocycloalkyl;
L is a bond, C₁-C₆ alkylene, C₁-C₆ heteroalkylene, S, O, or NH;
E represents a structure of Formula (Ic), 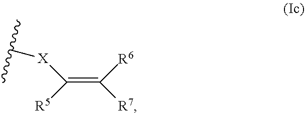
wherein,
X is C(═O), P(═O)OR², C(═S), or S(═O)ₙ, where n is 1 or 2;
R² is hydrogen or optionally substituted C₁-C₃ alkyl;
R⁵ is hydrogen, cyano, halogen, C₁-C₆alkyl, C₁-C₆alkoxyl, C₁-C₆heteroalkyl, C₁-C₃cycloalkyl, or C₂-C₅heterocycloalkyl, each of the alkyl, alkoxyl, heteroalkyl, cycloalkyl or heterocycloalkyl is optionally substituted, and
R⁷ is hydrogen, cyano, halogen, C₁-C₆ alkyl, C₁-C₆ alkoxyl, C₁-C₆ heteroalkyl, C₃-C₆ cycloalkyl, C₂-C₇ heterocycloalkyl, C₁-C₉heteroaryl, —C₁-C₆alkylene-C₁-C₉heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl or heterocycloalkyl is optionally substituted; or
R⁵ and R⁷ taken together form a bond; or
R⁵ and R⁷ taken together with the carbon atoms to which they are attached form a 5-8 membered partially saturated cycloalkyl, wherein the cycloalkyl is optionally substituted;
R⁶ is hydrogen, cyano, halogen, C₁-C₆ alkyl, C₁-C₆ alkoxyl, C₁-C₆ heteroalkyl, C₃-C₆ cycloalkyl, C₁-C₃ heterocycloalkyl, C₁-C₉heteroaryl, —C₁-C₆alkylene-C₁-C₆heteoraryl, —C₁-C₆alkylene-NH(C₁-C₆ alkyl), or —C₁-C₆alkylene-N(C₁-C₆ alkyl)₂, each of the alkyl, alkylene, alkoxyl, heteroalkyl, cycloalkyl, heterocycloalkyl, or heteroaryl is optionally substituted;
R²⁸ and R²⁹ are each independently hydrogen, hydroxyl, C₁-C₆ alkyl, C₁-C₆ alkoxy, cyano, nitro, or C₃-C₆ cycloalkyl, or R²⁸ and R²⁹ taken together with the carbon atom to which they are attached form a 3-6 membered ring;
R³⁰ is selected from halogen, cyano, C₁-C₆ alkyl, C₁-C₆ alkoxy, C₁-C₆ heteroalkyl, C₃-C₁₀cycloalkyl, C₂-C₁₂heterocycloalkyl, aryl, C₁-C₉heteroaryl, —C₁-C₆alkylene-C₃-C₁₀cycloalkyl, —C₁-C₆alkylene-C₂-C₁₂heterocycloalkyl, —C₁-C₆alkylene-aryl, —C₁-C₆ alkylene-C₁-C₉heteroaryl, —C₁-C₆heteroalkylene-C₃-C₁₀cycloalkyl, —C₁-C₆heteroalkylene-C₂-C₁₂-heterocycloalkyl, —C₁-C₆heteroalkylene-aryl, —C₁-C₆alkylene-C₁-C₉heteroaryl wherein each of the alkyl, alkoxy, heteroalkyl, cycloalkyl, heterocycloalkyl, aryl, and heteroaryl is optionally substituted; and
R³³ is hydrogen, C₁-C₆ alkyl, C₁-C₆ heteroalkyl, or C₃-C₆cycloalkyl, wherein each of the alkyl, heteroalkyl, and cycloalkyl is optionally substituted.

65. The conjugate of any one of claims 60-64, wherein the metal chelator is selected from AAZTA, BAT, BAT-TM, Crown, Cyclen, DO2A, CB-DO2A, DO3A, H₃HP-DO3A, Oxo-DO3A, p-NH₂-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H decapa, bispa², H₄pypa, H₄octapa, H₄CHXoctapa, p-SCN-Bn-H₄octapa, p-SCN-Bn-H₄octapa, TTHA, p-NOz-Bn-neunpa, H₄octox, H₂macropa, H₂bispa², H₄phospa, H₅phospa, p-SCN-Bn-H₆phospa, TETA, p-NOrBn-TETA, TRAP, TPA, HBED, SHBED, HBED-CC, (HBED-CC)TFP, DMSA, DMPS, DHLA, lipoic acid, TGA, BAL, Bis-thioseminarabazones, p-SCN-NOTA, nNOTA, NODAGA, CB-TE1A1P, 3P-C-NETA-NCS, 3p-C-DEPA, 3P-C-DEPA-NCS, TCMC, PCTA, NODIA-Me, TACN, pycup1A1B, pycup2A, THP, DEDPA, H₂DEDPA, p-SCN-Bn-H₂DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, L^{py}, L¹, L², L³, and EuK-106.

66. The conjugate of any one of claims 60-65, wherein the metal chelator is 2,2′,2″,2′″-((2S,5S, 8S, 11 S)-2,5,8,11-tetramethyl-1,4,7,10-tetraazacyclododecane-1,4,7,10-tetrayl)tetraacetic acid.

67. The conjugate of any one of claims 60-65, wherein the metal chelator is ((2S,5 S, 8S, 11S)-2,5,8,11-tetraethyl-1.4,7,10-tetraazacyclododecane-1,4,7,10-tetrayl)tetraacetic acid.

68. The conjugate of any one of claims 60-65, wherein the metal chelator is a chelator in FIG. 1 to FIG. 15.

69. The conjugate of any one of claims 60-65, wherein the metal chelator is DOTA.

70. The conjugate of any one of claims 60-69, wherein the radionuclide is ⁶²Cu, ⁶⁴Cu, ⁶⁷Cu, ⁶⁸Ga, ⁸⁹Zr, ⁹⁹Y, ^{99m}Tc, ¹⁰⁵Rh, ¹¹¹In, ¹³⁴Ce, ¹⁴⁸Gd, ¹⁴⁹Tb, ¹⁵²Tb, ¹⁵³Pm, ¹⁶⁷Tm, ¹⁷⁵Yb, ¹⁷⁷Lu, ²⁰⁹Bi, ²¹²Pb, ¹²³Po, ²¹³Bi, ²²³Ra, ²²³Fr, ²²⁷Th, ²²⁵Ac, or ²²⁹Th.

71. A pharmaceutical composition comprising a radiolabeled compound or conjugate of any one of claims 1-70, and a pharmaceutically acceptable excipient or carrier.

72. A method of making a covalently modified KRAS protein in vivo, comprising administering a radiolabeled compound or conjugate of any one of claims 1-70 or a pharmaceutical composition of claim 71 to a subject, wherein the subject has a KRAS protein comprising a glycine to cysteine amino acid substitution at residue 12.

73. The method of claim 72, wherein the subject has a cancer.

74. A method of treating cancer in a subject in need thereof, comprising administering to the subject a radiolabeled compound or conjugate of any one of claims 1-70 or a pharmaceutical composition of claim 71.

75. The method of claim 73 or 74, wherein the cancer is selected from the group consisting of Cardiac cancer: sarcoma (angiosarcoma, fibrosarcoma, rhabdomyosarcoma, liposarcoma), myxoma, rhabdomyoma, fibroma, lipoma and teratoma; Lung cancer: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal cancer: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract cancer: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma): Liver cancer; hepatoma (hepatocellular carcinoma), cholangiocarcinoma, hepatoblastoma, angiosarcoma, hepatocellular adenoma, hemangioma: Biliary tract cancer: gall bladder carcinoma, ampullary carcinoma, cholangiocarcinoma: Bone cancer: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors: Nervous system cancer: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological cancer: uterus (endometrial 'carcinoma (serous cystadenocarcinoma, mucinous cystadenocarcinoma, unclassified carcinoma), granulosa-thecal cell tumors, Sertoli-Ley dig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), Hematologic cancer: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma): Skin cancer: malignant melanoma, basal cell carcinoma, squamous cell carcinoma, Kaposi's sarcoma, moles dysplastic nevi, lipoma, angioma, dermatofibroma, keloids, psoriasis; and Adrenal glands cancer: neuroblastoma.

76. The method of any one of claims 73 to 75, wherein the cancer is non-small cell lung cancer.

77. The method of any one of claims 74 to 76, wherein the method comprises administering
(i) a first radiopharmaceutical conjugate comprising a radionuclide configured for companion diagnostic and
(ii) a second radiopharmaceutical conjugate comprising a radionuclide selected from an alpha or beta-particle emitter,
wherein the first and the second radiopharmaceutical conjugates have the same structure except for the radionuclide.

78. The method of claim 77, wherein the radionuclide of the first radiopharmaceutical conjugate is selected from ⁶²Cu, ⁶⁴Cu, ⁸⁹Zr, ¹³⁴Ce, ¹⁵²Tb, ⁶⁸Ga, ¹¹¹In, and ^{99m}Tc.

79. The method of claim 77, wherein the radionuclide of the first radiopharmaceutical conjugate is selected from ⁶²C, ¹³N, ¹⁵O, ¹⁸F, ⁷⁴As, ⁷⁶Br, ¹²⁹I, ¹²⁴I, and ¹²⁵I.

80. The method of claim 77, wherein the radionuclide of the second radiopharmaceutical conjugate is selected from ²²⁵Ac, ²¹³Bi, ²⁰⁹Bi, ¹⁴⁹Tb, ²²³Ra, ²²⁷Th, ²²³Fr, ¹⁴⁸Gd, ²²⁹Th ²¹³Po, ⁶⁷Cu, ¹⁷⁷Lu, ⁹⁰Y, ²¹²Pb, ¹⁰⁵Rh, ¹⁷⁵Yb, ¹⁶⁷Tm, ¹⁵³Pm, and ¹¹¹In.

81. The method of claim 77, wherein the radionuclide of the second radiopharmaceutical conjugate is selected from ¹³¹I and ²¹¹ At.

82. A method of killing a cell harboring a G12C KRAS mutation, the method comprising contacting a cell harboring a G12C KRAS mutation with a radiolabeled compound or conjugate of any one of claims 1-70 or a pharmaceutical composition of claim 71, thereby delivering a dose of radiation to the cell.

83. A method of delivering a radionuclide to a cell comprising administering a radiolabeled compound or conjugate of any one of claims 1-70 or a pharmaceutical composition of claim 71.

84. The method of claim 83, wherein the radiolabeled compound or conjugate irreversibly binds to an intracellular protein of the cell.

85. A method of diagnosing cancer patients harboring a G12C KRAS mutation comprising administering to a patient a radiolabeled compound or conjugate of any one of claims 1-70 or a pharmaceutical composition of claim 71.

86. A method of imaging a cancer harboring a G12C KRAS mutation comprising administering to a patient a radiolabeled compound or conjugate of any one of claims 1-70 or a pharmaceutical composition of claim 71.

87. The method of claim 85 or 86, further comprising measuring the concentration of the radiolabeled compound or conjugate accumulated in the patient.

88. The method of any one of claims 85 to 87, further comprising measuring the amount of radiation emitted from the radionuclide.

89. The method of any one of claims 85 to 88, further comprising analyzing the elimination profile of the radiolabeled compound or conjugate in the patient.

90. The method of any one of claims 85 to 89, further comprising measuring an elimination half-life of the radiolabeled compound or conjugate in the patient.

91. A method of producing a compound having a structure of Formula (VIa), Formula (VIb), Formula (VIc), or Formula (VId) in vivo, comprising administering a radiolabeled compound of any one of claims 48-58 to a subject, 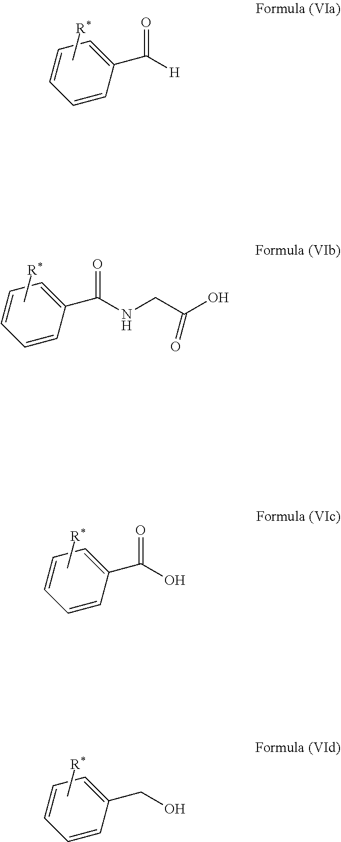
wherein
R* is fluorine-18 (¹⁸F), iodine-131 (¹³¹I), iodine-123 (¹²³I), iodine-124 (¹²⁴I), iodine-125(¹²⁵I), or astatine-211 (²¹¹At).

92. A method of excreting a compound having a structure of Formula (VIa), Formula (VIb), Formula (VIc), or Formula (VId) from a subject's body, comprising administering a radiolabeled compound of any one of any one of claims 48-58 to a subject, 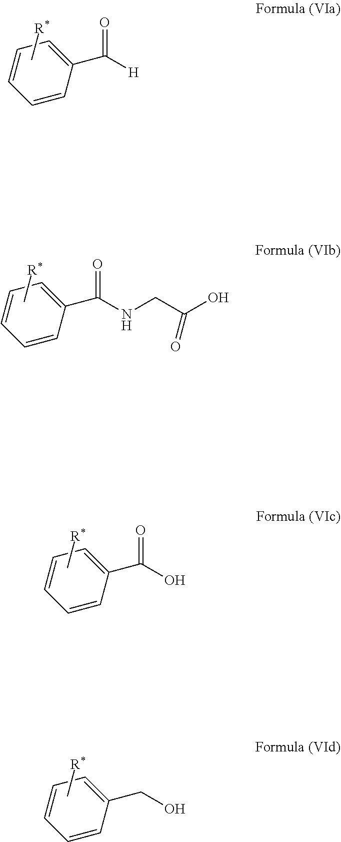
wherein
R* is fluorine-18 (¹⁸F), iodine-131 (¹³¹I), iodine-123 (¹²³I), iodine-124 (¹²⁴I), iodine-125 (¹²⁵I) or astatine-211 (²¹¹At).

93. The method of claim 91 or 92, wherein;
the compound having a structure of Formula (VIa) is 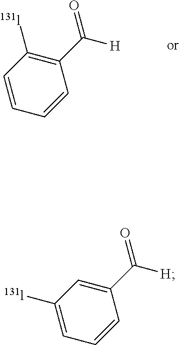
the compound having a structure of Formula (VIb) is 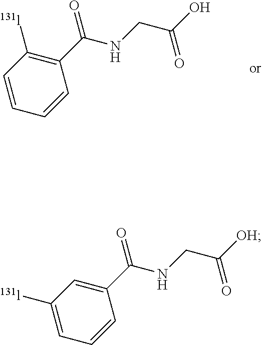
the compound having a structure of Formula (VIc) is 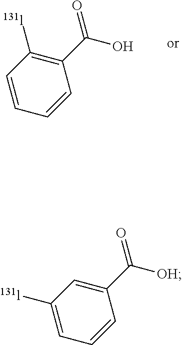
the compound having a structure of Formula (VId)

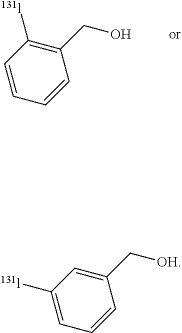

94. A covalently modified KRAS protein comprising,
a KRAS protein comprising a glycine to cysteine amino acid substitution at residue 12, and
a radiolabeled compound comprising a covalently bonded radioisotope, wherein the radiolabeled compound is bonded to the KRAS protein at the cysteine residue 12 of the KRAS protein through a covalent bond, and
wherein residue position numbering of the KRAS protein is based on SEQ ID NO: 1 or SEQ ID NO:2 as a reference sequence.

95. The covalently modified KRAS protein of claim 94, wherein the radiolabeled compound comprises the structure of a radiolabeled compound of any one of claims 1-58.

96. A conjugate comprising,
(a) a targeting ligand that is configured to form a covalent bond with a KRAS protein at the G12C position, wherein residue position numbering of the KRAS protein is based on SEQ ID NO: 1 or SEQ ID NO: 2; and
(b) a radionuclide, wherein the radionuclide is iodine-131 or astatine-211.

97. A conjugate comprising,
(a) a targeting ligand that is covalently bound to an intracellular mutated KRAS protein at the G12C position, wherein residue position numbering of the KRAS protein is based on SEQ ID NO: 1 or SEQ ID NO: 2; and
(b) a radionuclide.

98. The conjugate of claim 97, wherein the radionuclide is selected from is astatine-211, astatine-217, actinium-225, americium-243, radium-223, lead-212, lead-203, copper-64, copper-67, copper-60, copper-61, copper-62, bismuth-212, bismuth-213, gallium-68, gallium-67, dysprosium-154, gadolinium-148, gadolinium-153, samarium-140, samarium-147, samarium-153, terbium-149, thorium-227, thorium-229, iron-59, yttrium-86, indium-111, thorium-166, technetium-94, technetium-99^{m}, yttrium-90, lutetium-177, terbium-161, rhenium-186, rhenium-188, cobalt-55, scandium-43, scandium-44, scandium-47, dysprosium-166, fluorine-18, and iodine-131.

99. The conjugate of claim 96 or 97, wherein the radionuclide is iodine-131.

100. The conjugate of any one of claims 96 to 99, wherein the targeting ligand comprises an electrophilic functional group.

101. The conjugate of claim 100, wherein the electrophilic functional group comprises a structure of Formula (Ia) or Formula (Ib); 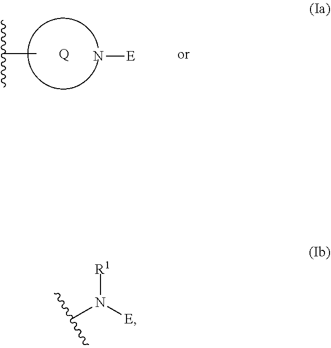
wherein,
ring Q is a 3 to 10 membered heterocycloalkylene, wherein Q is optionally substituted;
R¹ is hydrogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; and
E represents a structure of Formula (Ic), 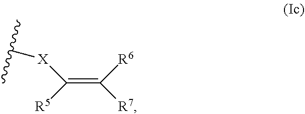
wherein,
X is C(═O), P(O)OR², C(═S), or S(═O)ₙ, where n is 1 or 2;
R² is hydrogen or substituted or unsubstituted C₁-C₃ alkyl;
R⁵ and R⁷ are each independently selected from hydrogen, —CN, halogen, substituted or unsubstituted C₁-C₆ alkyl, substituted or unsubstituted C₁-C₆ heteroalkyl, substituted or unsubstituted C₃-C₇ cycloalkyl, or substituted or unsubstituted C₂-C₇ heterocycloalkyl; or R⁵ and R⁷ taken together form a bond;
R⁶ is hydrogen, halogen, —CN, C₁-C₆ alkyl, C₂-C₅ heteroalkyl, heteroaryl, aryl, alkylaminylalkyl, dialkylaminylalkyl, cycloalkyl or heterocycloalkyl, each of which is optionally substituted.

102. The conjugate of claim 100 or 101, wherein the electrophilic functional group comprises a structure of Formula (Id), 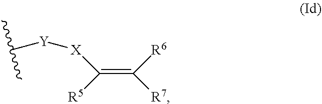
wherein,
X is C(═O), OC(═O), NR²C(═O), P(═O)OR², C(═S), S(═O)ₙ, OS(O)ₙ, NR²S(═O)ₙ, wherein n is 1 or 2;
Y is an alkylene, heteroalkylene, cycloalkylene, heterocycloalkylene, arylene, heteroarylene, or a bond, wherein each of the alkylene, heteroalkylene, cycloalkylene, heterocycloalkylene, arylene, or heteroarylene is optionally substituted;
each R² is independently hydrogen or substituted or unsubstituted C₁-C₃ alkyl;
R⁵ and R⁷ are each independently selected from hydrogen, cyano, halogen, substituted or unsubstituted C₁-C₆ alkyl, substituted or unsubstituted C₁-C₆, heteroalkyl, substituted or unsubstituted C₃-C₇ cycloalkyl, or substituted or unsubstituted C₂-C₆ heterocycloalkyl, or R⁵ and R⁷ taken together form a bond, and
R⁶ is hydrogen, halogen, —CN, C₁-C₆ alkyl, C₁-C₆heteroalkyl, heteroaryl, aryl, alkylaminylalkyl, dialkylaminylalkyl, cycloalkyl or heterocycloalkyl, each of which is optionally substituted.

103. The conjugate of claim 102, wherein E represents a structure of Formula (Ic) that is

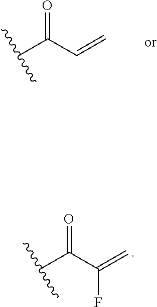

104. The conjugate of claim 102, wherein the electrophilic functional group is [chemical structure]
